SEQUENCE LISTING (7 sequences)

SEQ ID NO: 1 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 2 - protein, 1149 aa, Homo sapiens
MGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 3 - protein, 284 aa, Homo sapiens
SPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGK

SEQ ID NO: 4 - protein, 1152 aa, Synthetic construct
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQRLEACQLGTDDYDIPTTHHHHHH

SEQ ID NO: 5 - protein, 1338 aa, Synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGGGSGGGSSGGAIAMGQQPGKVLGDQRRPSLPALHFIKGAGKKESSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 6 - protein, 311 aa, Synthetic construct
MGHHHHHHHHHHSSGVDLGTENLYFQSSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGK

SEQ ID NO: 7 - protein, 12 aa, Synthetic construct
EAIYAAPFAKKK

(19) United States
(12) Patent Application Publication
(10) Pub. No.: US 2026/0297035 A1
(43) Pub. Date: Oct. 1, 2026

(54) PROTEIN KINASE INHIBITORS AND USES THEREOF

(71) Applicant: DELiver Therapeutics, Inc., Surfside, FL (US)

(72) Inventors: Kevin Slawin, Surfside, FL (US); David Spencer, Houston, TX (US); Damian Young, Houston, TX (US); Conrad Santini, Houston, TX (US); Michael Corsello, Houston, TX (US)

(21) Appl. No.: 19/161,956

(22) PCT Filed: Feb. 29, 2024
(86) PCT No.: PCT/US2024/017889

§ 371 Date: Sep. 3, 2025

Related U.S. Application Data
(60) Provisional application No. 63/488,437, filed on Mar. 3, 2023.
(60) Provisional application No. 63/554,862, filed on Feb. 16, 2024.

(51) Int. Cl. C07D 239/84; A61K 31/517; A61K 31/5377; A61P 35/00; C07D 401/12; C07D 401/14; C07D 403/10; C07D 403/12; C07D 405/12; C07D 413/10; C07D 413/12

(52) U.S. Cl. CPC C07D 239/84; A61K 31/517; A61K 31/5377; A61P 35/00; C07D 401/12; C07D 401/14; C07D 403/10; C07D 403/12; C07D 405/12; C07D 413/10; C07D 413/12

(57) ABSTRACT

Provided in part herein are protein kinase inhibitors having a structure according to specified Formulae, and uses thereof.

### RELATED APPLICATION

This patent application is related to U.S. provisional patent application No. 63/488,437, filed on Mar. 3, 2023, entitled PROTEIN TYROSINE KINASE INHIBITORS, naming Kevin SLAWIN et al. as inventors, and designated by attorney docket no. 061897-502P01 US and to U.S. provisional patent application No. 63/554,862, filed on Feb. 16, 2024, entitled PROTEIN KINASE INHIBITORS AND USES THEREOF, naming Kevin SLAWIN et al. as inventors, and designated by attorney docket no. 05337.0002 US02. The entire content of both the foregoing patent applications are incorporated herein by reference for all purposes, including all text, tables and drawings.

### REFERENCE TO A SEQUENCE LISTING

This application contains a Sequence Listing in computer readable form with file name 05337.0002WO01.xml (created Feb. 29, 2024, 13 kb). The computer readable form is incorporated herein by reference.

### FIELD

The technology relates in part to compounds that contain a quinazolinyl group and an amine-linked phenyl group. Compounds herein can inhibit one or more protein kinases (PKs), and the technology relates in part to uses of compounds herein.

### BACKGROUND

Protein kinases (PKs) are a category of proteins that catalyze phosphorylation of different protein substrates. Each PK typically is referred to by the three or four letter code afforded to the gene that encodes the PK protein. PKs are categorized into PK families according to polypeptide sequence and/or function. Dysregulation of PK genes and their encoded PKs are associated with certain cancers. The ABL1 protooncogene, for example, which also is known as ABL, JTK7, p150, c-ABL, CHDSKM, c-ABL1, encodes an ABL1 PK implicated in processes of cell differentiation, cell division, cell adhesion, and stress response. Activity of the ABL1 PK is negatively regulated by its SH3 polypeptide domain. Modification to the ABL1 protooncogene, such as deletion of the SH3 domain-encoding portion and fusion with another protein, for example, can turn it into an oncogene. Translocation and head-to-tail fusion of the BCR and ABL1 genes is present in cases of chronic myeloid leukemia (CML) and in a subset of acute lymphoblastic leukemia (i.e., Philadelphia chromosome-positive ALL). Genes encoding other PKs, which can phosphorylate protein targets different than those of ABL1, can be dysregulated in certain cancers and other medical conditions.

### SUMMARY

Provided are compounds that inhibit one or more particular PKs, including ABL, BTK, AURK, JAK, TRK, RET, EPH, TNK, PLK, IRAK and TYK family PKs, and may be used for treating cancers and other medical conditions. Compounds herein contain a quinazolinyl group, an amine-linked phenyl group and substituents that can afford PK inhibitory activity. Compounds herein can inhibit multiple PKs in certain embodiments. Compounds herein can selectively inhibit at least one PK in certain instances. Compounds that inhibit one or more particular protein kinases (PKs) can be used to treat cancers and other medical conditions.

### BRIEF DESCRIPTION OF THE DRAWINGS

The drawings illustrate certain embodiments of the technology and are not limiting. For clarity and ease of illustration, the drawings are not made to scale, and, in some instances, various aspects may be shown exaggerated or enlarged to facilitate an understanding of particular embodiments.

FIG. 1 illustrates processes of a protein kinase (PK) labeled peptide cleavage assay.

FIG. 2 illustrates amino acid substitutions identified in certain ABL1 variants.

FIG. 3 shows ABL1 inhibition by Cmpd10 and Cmpd11 determined by a labeled peptide cleavage assay.

FIG. 4A to FIG. 11C show PK inhibition by compounds determined by peptide cleavage and tracer displacement assays. FIG. 4A and FIG. 4B shows inhibition of ABL family PKs.

FIG. 5 shows inhibition of BTK family PKs.

FIG. 6 shows inhibition of AURK family PKs.

FIGS. 7A and 7B show inhibition of JAK family PKs.

FIG. 8 shows inhibition of TRK family PKs.

FIG. 9 shows inhibition of RET family PKs.

FIG. 10A shows inhibition of EPH family PKs. FIG. 10B shows inhibition of TNK family, PLK family and IRAK family PKs. FIG. 10C shows inhibition of SRC and DDR family PKs. FIG. 10D shows inhibition of ABL2 and PTK2B PKs.

FIG. 11A shows inhibition of ABL and BTK family PKs. FIG. 11B shows inhibition of ABL and AURK family PKs. FIG. 11C shows inhibition of ABL, BTK and AURK family PKs.

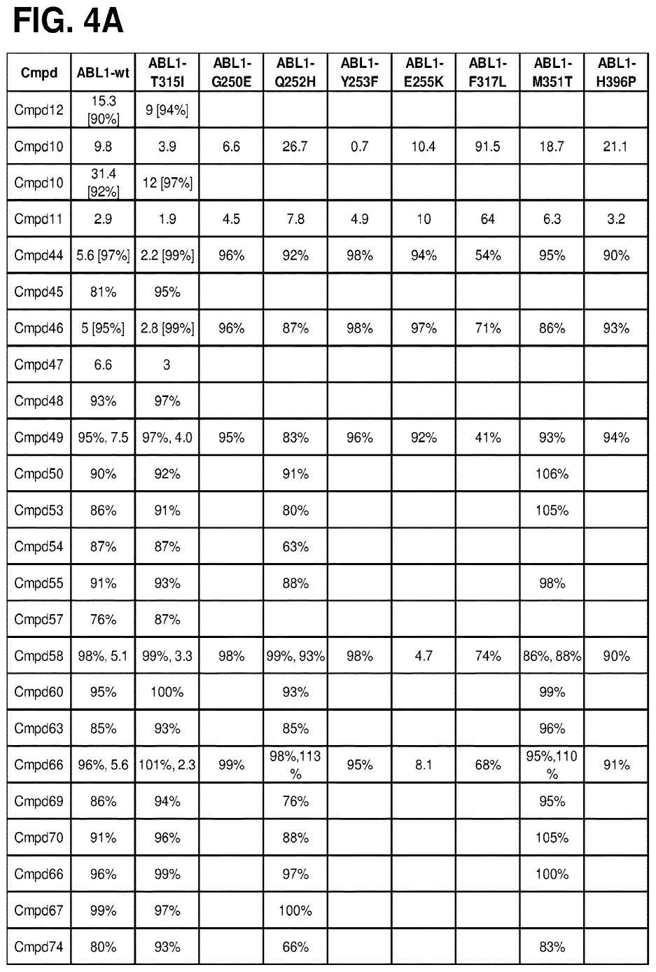

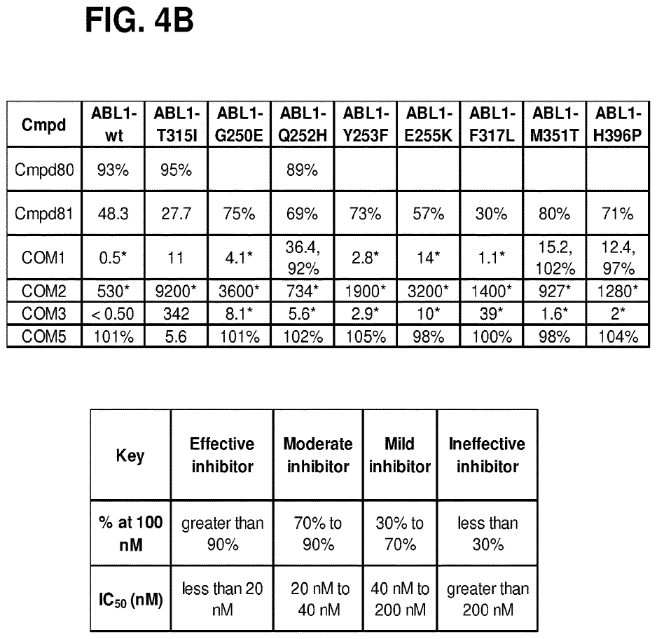

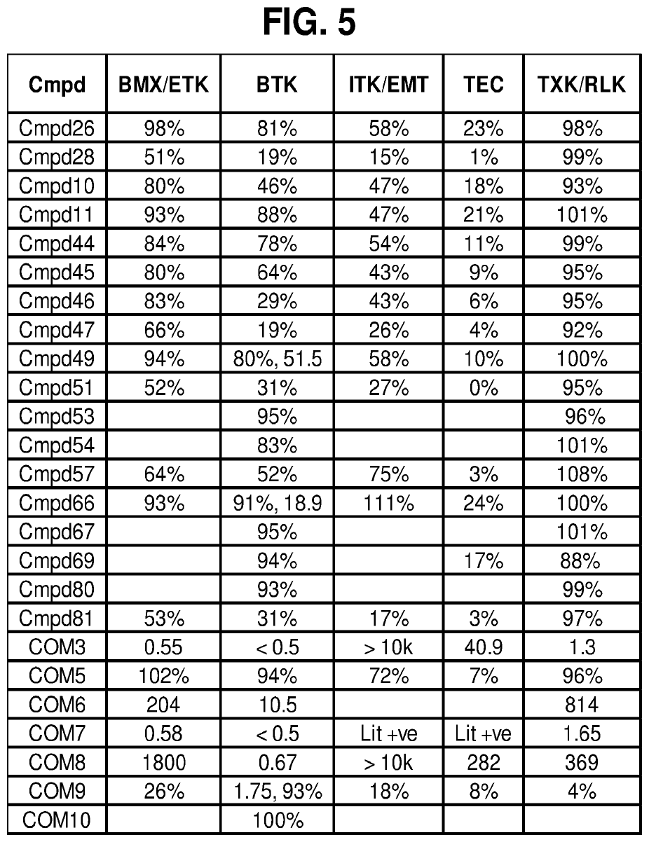

The legend at the bottom of FIG. 4B is applicable to charts in FIG. 5 to FIG. 11C. FIG. 12A and FIG. 12B show observed cytotoxicity responses to compounds for K562 LD₅₀ and NALM-21 cells, respectively. FIG. 13A, FIG. 13B and FIG. 13C show observed IL-6 reduction responses to compounds. FIG. 14 illustrates a dose escalation study overview.

### DETAILED DESCRIPTION

Compounds herein can inhibit one or more protein kinases (PKs) and can be used for treatment of medical conditions including cancers. Development of PK inhibitor cancer treatments is challenging. PK inhibitor drugs can present issues including ineffectiveness in a patient subgroup, loss of therapeutic effect during treatment for a patient subgroup, and triggering a serious adverse event in a patient subgroup. For example, an amino acid substitution in the ABL1 kinase domain occurring in a patient can impart resistance to cancer treatment. An ABL1 variant containing a threonine 315 to isoleucine (13151) amino acid substitution can result in resistance to cancer treatment, for example. It has been reported that developing inhibitors against this variant is challenging because inhibitor candidates often bind to the PK at or near the site of the amino acid substitution. Despite this challenge, certain compounds herein can effectively inhibit the ABL1 (T315I) variant. Certain compounds herein can effectively inhibit other PKs, can effectively inhibit multiple PKs in different PK families and/or can selectively inhibit a PK.

### Compounds

In certain aspects, provided is a compound of Formula A1 or Formula A2:

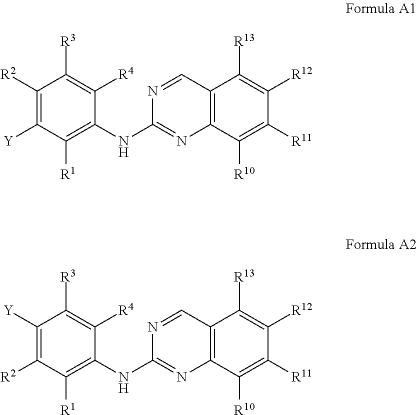

or a pharmaceutically acceptable salt, amide or ester thereof, where:
R¹, R², R³ and R⁴ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino or amido;
Y is —N(R^{b})C(O)—R^{Y}, —C(O)N(R^{b})—R^{YA}, —N(R^{b})—CH₂—R^{Y}; —CH₂—N(R^{b})R^{Y}, —N(R^{a})C(O)—R^{V}—N(R^{b})R^{Y}, —N(R^{b})C(O)—R^{V}—R^{Y}, —N(R^{a})R^{b} or of Formula F:

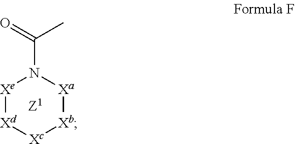

R^{a} is hydrogen, optionally substituted alkyl, optionally substituted alkynyl or of Formula F;
R^{b} is hydrogen or optionally substituted alkyl;
R^{V} is an optionally substituted alkylene;
R^{Y} is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted cycloalkylalkyl, optionally substituted heterocycloalkylalkyl, optionally substituted aryl, optionally substituted arylaminoalkyl, optionally substituted aryloxyalkyl, optionally substituted arylthioalkyl or optionally substituted arylalkyl, optionally substituted heteroaryl, optionally substituted heteroarylaminoalkyl, optionally substituted heteroaryloxyalkyl, optionally substituted heteroarylthioalkyl, optionally substituted heteroarylalkyl or is of Formula F;
R^{YA} is an optionally substituted aryl, optionally substituted arylaminoalkyl, optionally substituted aryloxyalkyl, optionally substituted arylthioalkyl or optionally substituted arylalkyl, optionally substituted heteroaryl, optionally substituted heteroarylaminoalkyl, optionally substituted heteroaryloxyalkyl, optionally substituted heteroarylthioalkyl or optionally substituted heteroarylalkyl;
Z¹ is an optionally substituted heterocycloalkyl, X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A}; and R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R¹⁰, R¹¹, R¹² and R¹³ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino, amido, R^{W} or —W—R^{W};
R^{W} is an optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl;
W is an optionally substituted alkylene, optionally substituted alkynyl, amino, amido, —O—, —S—, —S(O)— or —SO₂—;
R^{z} is hydrogen or R^{u}; and
R^{u} is an optionally substituted alkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl.

A substituent having a structure according to Formula F is referred to herein as a “Formula F group.” Z¹, and Z², Z³, Z⁴, Z⁵, Z^{2a}, Z^{3a}, Z^{4a}, Z^{5a}, Z^{2b}, Z^{3b}, Z^{4b} and Z^{5b} designations herein, each designates a ring structure (e.g., monocyclic or multicyclic ring structure) and is not an atom, and the designated ring structure may be substituted or unsubstituted.

A compound herein is with the proviso that it is not of Formula X1, not of Formula X2, not of Formula X3 and not of Formula X4:

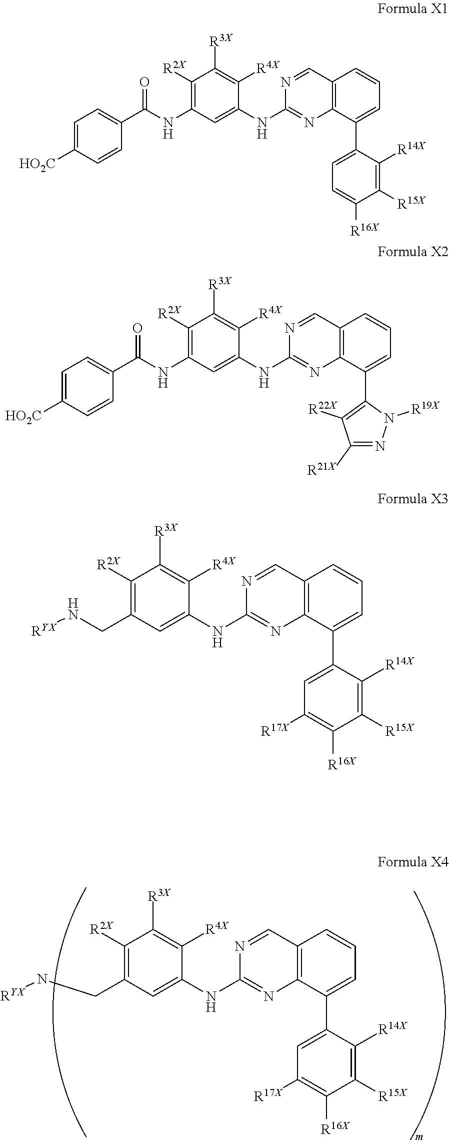

where: R^{2X}, R^{3X} and R^{4X} each independently is hydrogen, optionally substituted C1-C4 alkyl or optionally substituted C1-C4 alkoxy; R^{14X}, R^{15X}, R^{16X} and R^{11X} each independently is hydrogen, optionally substituted C1-C4 alkyl, optionally substituted C1-C4 alkoxy, or halo; R^{19X}, R^{21X} and R^{22X} each independently is hydrogen or optionally substituted C1-C6 alkyl; m is an integer of 1 or 2; R^{YX} is methyl or

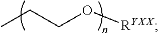

n is an integer of 1 to 10; and R^{YXX} is hydrogen, optionally substituted alkyl or optionally substituted amidoalkyl. Compounds of Formula X1, Formula X2, Formula X3 and Formula X4 as defined are excluded.

In certain aspects pertaining to a compound of Formula A1 or Formula A2, Y is —N(R^{b})C(O)—R^{Y} or —C(O)N(R^{b})—R^{YA}, and R^{Y} and R^{YA} each is an optionally substituted phenyl. In certain aspects, provided is a compound of Formula A1-1, Formula A1-2 or Formula A2-1:

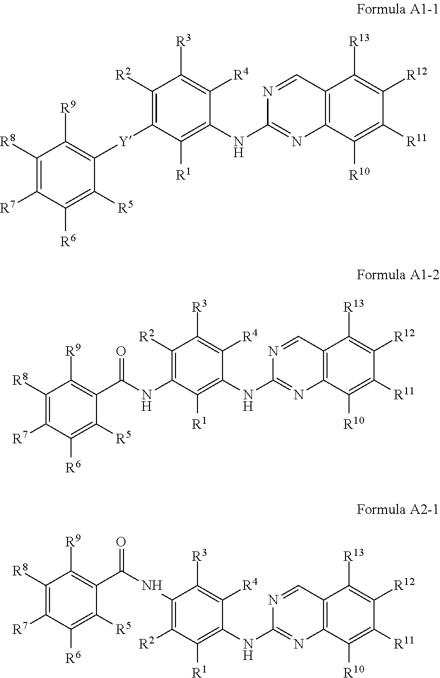

or a pharmaceutically acceptable salt, amide or ester thereof, where:
Y′ is —N(R^{b})C(O)— or —C(O)N(R^{b})—;
R^{b}, R¹, R², R³, R⁴, R¹⁰, R¹¹, R¹² and R¹³ each is as defined for a compound of Formula A1 and Formula A2;
R⁵, R⁶, R⁷, R⁸, and R⁹ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —B(OH)₂, —C(O)R^{z}, —C(O)OH, —C(O)OR^{u}, hydroxy, halo, cyano, nitro, amino, amido, optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl or a Formula F group; and R^{z} and R^{u} each is as defined for Formula A1 and Formula A2.

In certain aspects pertaining to a compound of Formula A1, Formula A1-1, Formula A1-2, Formula A2 or Formula A2-1, R¹⁰ is R^{W} and R^{W} is an optionally substituted phenyl or optionally substituted pyrazolyl. In certain aspects, provided is a compound of Formula A1-3, Formula A1-4, Formula A1-5, Formula A1-6, Formula A2-2 or Formula A2-3:

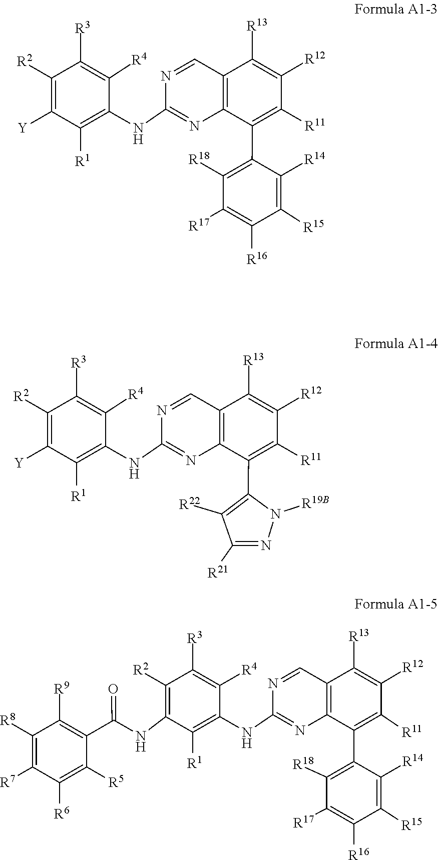

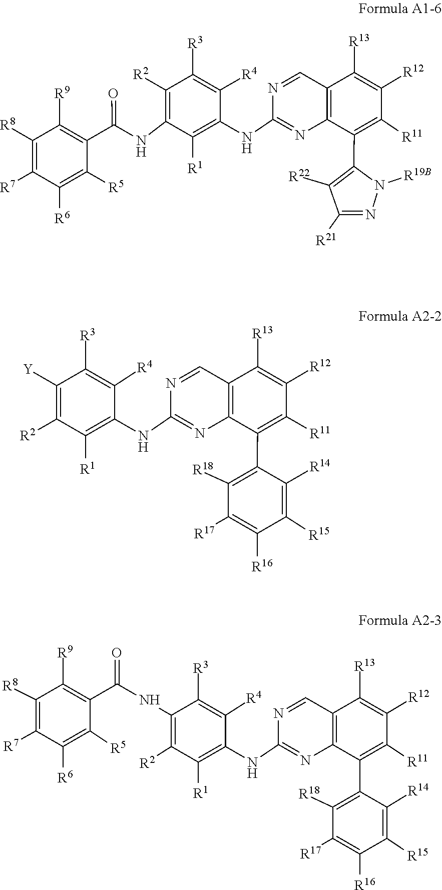

or a pharmaceutically acceptable salt, amide or ester thereof, where:
Y, R¹, R², R³, R⁴, R⁵, R⁶, R⁷, R⁸, R⁹, R¹¹, R¹² and R¹³ each is as defined for a compound of Formula A1, Formula A1-1, Formula A1-2, Formula A2 or Formula A2-1;
R¹⁴, R¹⁵, R¹⁶, R¹⁷, R¹⁸, R^{19B}, R²¹ and R²² each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl; and R^{z} and R^{u} each is as defined for Formula A1 and Formula A2.

In certain aspects pertaining to a compound of Formula A1 or Formula A1-2, R¹⁰ is —W—R^{W}, W is —C≡C—, and R^{W} is an optionally substituted phenyl. In certain aspects, provided is a compound of Formula A1-7 or Formula A1-8:

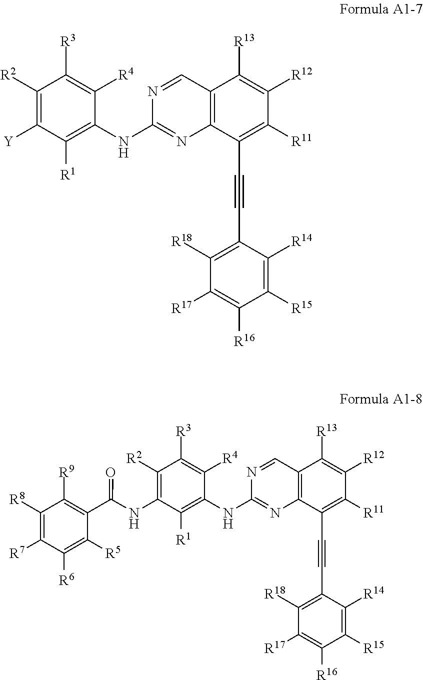

or a pharmaceutically acceptable salt, amide or ester thereof, where: R¹, R², R³, R⁴, R⁵, R⁶, R⁷, R⁸, R⁹, R¹¹, R¹², R¹³, R¹⁴, R¹⁵, R¹⁶, R¹⁷ and R¹⁸ each is as defined for a compound of the preceding Formulae.

In certain aspects, a compound herein is provided as a hydrochloride salt.

Certain features of compounds of Formula A1, Formula A1-1, Formula A1-2, Formula A1-3, Formula A1-4, Formula A1-5, Formula A1-6, Formula A1-7, Formula A1-8, Formula A2, Formula A2-1, Formula A2-2 and Formula A2-3 are described hereafter.

### Y Substituents

In certain embodiments, R^{Y} and R^{YA} each is an optionally substituted aryl, optionally substituted arylalkyl, optionally substituted heteroaryl or optionally substituted heteroarylalkyl. An optionally substituted arylalkyl typically contains an optionally substituted alkylene (for example, an optionally substituted C1-C6 alkylene) linked to an optionally substituted aryl. An optionally substituted heteroarylalkyl typically contains an optionally substituted alkylene (for example, an optionally substituted C1-C6 alkylene) linked to an optionally substituted heteroaryl. In certain embodiments, R¹ and R^{YA} each contains or is an optionally substituted aryl or optionally substituted heteroaryl of Formula B1:

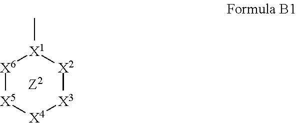

where: Z² is aryl or heteroaryl; X¹ independently is C or N; X² independently is C—R⁵, N—R^{5B} or N; X³ independently is C—R⁶, N—R^{6B} or N; X⁴ independently is C—R⁷, N—R^{7B} or N; X⁵ independently is C—R⁸, N—R^{8B} or N; and X⁶ independently is C—R⁹, N—R^{9B} or N; R⁶, R⁷, R⁸, R⁹, R^{6B}, R^{7B}, R^{8B} and R^{9B} are as defined herein; and optionally two adjacent R⁶, R⁷, R⁸, R⁹, R^{6B}, R^{7B}, R^{8B} and R^{9B} are linked in an optionally substituted aryl or optionally substituted heteroaryl. In certain embodiments pertaining to Formula B1, Z² is an optionally substituted pyridinyl, optionally substituted pyrimidinyl, optionally substituted pyrazinyl, optionally substituted pyridazinyl or optionally substituted triazinyl. In certain embodiments pertaining to Formula B1, X¹ is C, X² is C—R⁵, X³ is C—R⁶, X⁴ is C—R⁷, X⁵ is C—R⁸, and X⁶ is C—R⁹. In certain embodiments pertaining to Formula B1, X¹ is C, X² is C—R⁵, X³ is N, X⁴ is C—R⁷, X⁵ is C—R⁸, and X⁶ is C—R⁹. In certain embodiments pertaining to Formula B1, X¹ is C, X² is N, X³ is C—R⁶, X⁴ is N, X⁵ is C—R⁸, and X⁶ is C—R⁹. In certain embodiments pertaining to Formula B1, X¹ is C, X² is C—R⁵, X³ is N, X⁴ is C—R⁷, X⁵ is N, and X⁶ is C—R⁹. In certain embodiments pertaining to Formula B1, X¹ is C, X² is C—R⁵, X³ is C—R⁶, X⁴ is N, X⁷ is C—R⁸ and X⁶ is C—R⁹.

In certain embodiments, R^{Y} and R^{YA} each contains or is an optionally substituted aryl or optionally substituted heteroaryl of Formula C1:

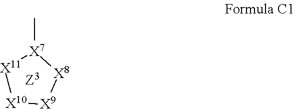

where:
Z³ is aryl or heteroaryl; X⁷ independently is C or N; X⁸ independently is C—R⁵, N—R^{5B}, N, O, S, S(O) or SO₂; X⁹ independently is C—R⁶, N—R^{6B}, N, O, S, S(O) or SO₂; X¹⁰ independently is C—R⁷, N—R^{7B}, N, O, S, S(O) or SO₂; and X¹¹ independently is C—R⁸, N—R^{8B}, N, O, S, S(O) or SO₂; R⁵, R⁶, R⁷, R⁸, R^{5B}, R^{6B}, R^{7B} and R^{8B} are as defined herein; and optionally two adjacent R⁵, R⁶, R⁷, R⁸, R^{5B}, R^{6B}, R^{7B} and R^{8B} are linked in an optionally substituted aryl or optionally substituted heteroaryl. In certain embodiments pertaining to Formula C1, Z³ is an optionally substituted pyrrolyl, optionally substituted imidazolyl, optionally substituted pyrazolyl, optionally substituted puranyl, optionally substituted thiophenyl, optionally substituted oxazolyl, optionally substituted thiazolyl, optionally substituted isoxazolyl or optionally substituted isothiazolyl. In certain embodiments pertaining to Formula C1:
X⁷ is C; X⁸ is N or N—R^{5B}; X⁹ is N or N—R^{6B}; X¹⁰ is C—R⁷; and X¹¹ is C—R⁸;
X⁷ is C; X⁸ is N or N—R^{5A}; X⁹ is C—R⁶; X¹⁰ is C—R⁷; and X¹¹ is C—R⁸;
X⁷ is C; X⁸ is N or N—R^{5B}; X⁹ is C—R⁶; X¹⁰ is C—R⁷; and X¹¹ is N or N—R^{8B};
X⁷ is C; X⁸ is N; X⁹ is C—R⁶; X¹⁰ is C—R⁷; and X¹¹ is S;
X⁷ is C; X⁸ is N; X⁹ is C—R⁶; X¹⁰ is C—R⁷; and X¹¹ is O; or
X⁷ is C; X⁸ is N; X⁹ is C—R⁶; X¹⁰ is O; and X¹¹ is C—R⁸.

In certain embodiments, R^{Y} is an optionally substituted cycloalkyl, optionally substituted cycloalkylalkyl, optionally substituted heterocycloalkyl or an optionally substituted heterocycloalkylalkyl. In certain embodiments, R^{Y} is an optionally substituted cycloalkylalkyl comprising an optionally substituted alkylene, for example an optionally substituted C1-C6 alkylene, linked to an optionally substituted cycloalkyl. In certain embodiments, R^{Y} is an optionally substituted heterocycloalkylalkyl comprising an optionally substituted alkylene, for example an optionally substituted C1-C6 alkylene, linked to an optionally substituted heterocycloalkyl.

In certain embodiments, R^{Y} is an optionally substituted heterocycloalkyl or an optionally substituted heterocycloalkylalkyl, and the optionally substituted heterocycloalkyl contains 4, 5 or 6 ring atoms. In certain instances, R^{Y} contains or is an optionally substituted piperidinyl, optionally substituted piperazinyl, optionally substituted tetrahydropyran, or optionally substituted morpholinyl, optionally substituted pyrrolidinyl, optionally substituted imidazolidinyl, optionally substituted pyrazolidinyl or optionally substituted azetidinyl. In certain instances, R^{Y} contains or is a substituted heterocycloalkyl containing 4, 5 or 6 ring atoms, which sometimes is a C1-C6 alkyl substituted heterocycloalkyl, a methyl substituted heterocycloalkyl or a mono-methyl substituted heterocycloalkyl.

In certain embodiments, R^{Y} is an optionally substituted cycloalkyl or optionally substituted heterocycloalkyl of Formula D1, or an optionally substituted cycloalkylalkyl or optionally substituted heterocycloalkylalkyl containing the optionally substituted cycloalkyl or optionally substituted heterocycloalkyl of Formula D1:

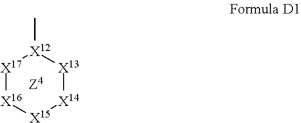

where: Z⁴ is cycloalkyl or heterocycloalkyl; X¹² is C—R^{aA}, C or N; X¹³ independently is C(R⁵)R^{5A}, C—R⁵, N—R^{5B}, N, O, S, S(O) or SO₂; X¹⁴ independently is C(R⁶)R^{6A}, C—R⁶, N—R^{6B}, N, O, S, S(O) or SO₂; X¹⁵ independently is C(R⁷)R^{7A}, C—R⁷, N—R^{7B}, N, O, S, S(O) or SO₂; X¹⁶ independently is C(R⁸)R^{8A}, C—R⁸, N—R^{8B}, N, O, S, S(O) or SO₂; and X¹⁷ independently is C(R⁹)R^{9A}, C—R⁹, N—R^{9B}, N, O, S, S(O) or SO₂; and optionally two adjacent R⁵, R^{5A}, R⁶, R^{6A}, R⁷, R^{7A}, R⁸, R^{8A}, R⁹ and R^{9A} are linked in an optionally substituted cycloalkyl or optionally substituted heterocycloalkyl. In certain instances pertaining to Formula D1, Z⁴ is an optionally substituted piperidinyl, optionally substituted piperazinyl, optionally substituted morpholinyl or optionally substituted tetrahydropyranyl. In certain instances pertaining to Formula D1, X¹² is C—R^{aA}; X¹³ is C—R⁵; X¹⁴ is C(R⁶)R^{6A}; X¹⁵ is N—R^{7B}; X¹⁶ is C(R⁸)R^{8A}; and X¹⁷ is C(R⁹)R^{9A}. In certain instances pertaining to Formula D1, X¹² is N; X¹³ is C(R⁵)R^{5A}; X¹⁴ is C(R⁶)R^{6A}; X¹⁵ is N—R^{7B}, X¹⁶ is C(R⁸)R^{8A}, and X¹⁷ is C(R⁹)R^{9A}. In certain instances pertaining to Formula D1, X¹² is N; X¹³ is C(R⁵)R^{5A}; X¹⁴ is C(R⁶)R^{6A}; X¹⁵ is C(R⁷)R^{7A}, X¹⁶ is C(R⁸)R^{8A}, and X¹⁷ is C(R⁹)R^{9A}. In certain instances pertaining to Formula D1, X¹² is C—R^{aA}; X¹³ is C(R⁵)R^{5A}; X¹⁴ is C(R⁶)R^{6A}; X¹⁵ is N—R^{7B}; X¹⁶ is C(R⁸)R^{8A}; and X¹⁷ is C(R⁹)R^{9A}. In certain instances pertaining to Formula D1, X¹² is C—R^{aA}; X¹³ is C(R⁵)R^{5A}; X¹⁴ is C(R⁶)R^{6A}; X¹⁵ is O; X¹⁶ is C(R⁸)R^{8A}; and X¹⁷ is C(R⁹)R^{9A}.

In certain embodiments, R^{Y} contains or is an optionally substituted cycloalkyl or optionally substituted heterocycloalkyl of Formula E1:

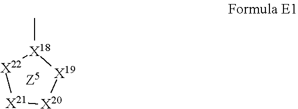

where: Z⁵ is cycloalkyl or heterocycloalkyl; X¹⁸ is C—R^{aA}, C or N; X¹⁹ independently is C(R⁵)R^{5A}, C—R⁵, N—R^{5B}, N, O, S, S(O) or SO₂; X²⁰ independently is C(R⁶)R^{6A}, C—R⁶, N—R^{6B}, N, O, S, S(O) or SO₂; X²¹ independently is C(R⁷)R^{7A}, C—R⁷, N—R^{7B}, N, O, S, S(O) or SO₂; and X²² independently is C(R⁸)R^{8A}, C—R⁸, N—R^{8B}, N, O, S, S(O) or SO₂; and optionally two adjacent R⁵, R^{5A}, R⁶, R^{6A}, R⁷, R^{7A}, R⁸, R^{8A}, R^{5B}, R^{6B}, R^{7B} and R^{8B} are linked in an optionally substituted cycloalkyl or optionally substituted hetero heterocycloalkyl. In certain embodiments pertaining to Formula E1, Z⁵ is an optionally substituted pyrrolidinyl, optionally substituted pyrazolidinyl, optionally substituted imidazolidinyl or optionally substituted pyrazolidinyl. In certain embodiments pertaining to Formula E1, X¹⁸ is C—R^{aA}, X¹⁹ is N—R^{5B}, X²⁰ is C(R⁶)R^{6A}, X²¹ is C(R⁷)R^{7A} and X²² is C(R⁸)R^{8A}.

In certain embodiments, Y is —N(R^{b})C(O)—R^{Y} or —N(R^{b})C(O)—R^{V}—R^{Y}, and R^{b}, R^{V} and R^{Y} each is as defined for Formula A1 and Formula A2.

In certain embodiments, R^{aA}, R⁵, R^{5A}, R⁶, R^{6A}, R⁷, R^{7A}, R⁸, R^{8A}, R⁹ and R^{9A} each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; R^{u} is as defined for Formula A1; and
R^{5B}, R^{6B}, R^{7B}, R^{8B}, and R^{9B} each independently is hydrogen, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl; and
In certain embodiments, R^{aA}, R⁵, R^{5A}, R⁶, R^{6A}, R⁷, R^{7A}, R⁸, R^{8A}, R⁹ and R^{9A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{c}C(O)N(R^{d})—, —C(O)N(R^{c}R^{d}), —NR^{e}R^{f}, —C(O)R^{z}, —C(O)OH, —C(O)OR^{u}, hydroxy, halo, cyano, nitro, optionally substituted C5-C6 cycloalkyl, optionally substituted heterocycloalkyl containing 5 or 6 ring member atoms; where R^{c}, R^{d}, R^{e} and R^{f} each independently is hydrogen or optionally substituted C1-C6 alkyl. In certain instances, an optionally substituted C1-C6 haloalkyl is an optionally substituted C1-C4 haloalkyl, or trifluoromethyl.

In certain instances, R^{c}, R^{d}, R^{e} and R^{f} each independently is hydrogen or methyl. In certain instances, R^{u} is an optionally substituted C1-C4 alkyl, butyl, tert-butyl, iso-butyl, propyl, iso-propyl, ethyl or methyl. In certain instances pertaining to Formula B1, Formula C1, Formula D1 and Formula E1, R^{aA}, R⁵, R^{5A}, R⁶, R^{6A}, R⁷, R^{7A}, R⁸, R^{8A}, R⁹ and R^{9A} each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, R^{5B}, R^{6B}, R^{7B}, R^{8B}, and R^{9B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u}. In certain instances, R^{u} is an optionally substituted C1-C4 alkyl, butyl, tert-butyl, iso-butyl, propyl, iso-propyl, ethyl or methyl. In certain instances pertaining to Formula B1, Formula C1, Formula D1 and Formula E1, R^{5B}, R^{6B}, R^{7B}, R^{8B}, and R^{9B} each independently is hydrogen, optionally substituted C1-C4 alkyl, butyl, tert-butyl, iso-butyl, propyl, iso-propyl, ethyl or methyl.

In certain embodiments, Y contains a heterocycloalkyl of Formula D1 and is:

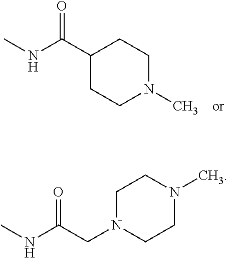

In certain embodiments for which Y contains an optionally substituted aryl, optionally substituted heteroaryl, optionally substituted cycloalkyl or optionally substituted heterocycloalkyl of Formula B1, C1, D1 or E1, one or more of R^{aA}, R⁵, R^{5A}, R⁶, R^{6A}, R⁷, R^{7A}, R⁸, R^{8A}, R⁹ and R^{9A} is —C(O)N(R^{c}R^{d}) where R^{c} and R^{d} each independently is hydrogen or optionally substituted C1-C4 alkyl. In certain embodiments, at least R⁷ is —C(O)N(R^{c}R^{d}) and R^{c} and R^{d} each independently is hydrogen or optionally substituted C1-C4 alkyl. In certain instances, R^{c} and R^{d} each independently is unsubstituted C1-C4 alkyl. In certain instances, at least R⁷ is —C(O)N(CH₃)CH₃, —C(O)N(H)CH₃ or —C(O)NH₂.

In certain embodiments for which Y contains an optionally substituted aryl, optionally substituted heteroaryl, optionally substituted cycloalkyl or optionally substituted heterocycloalkyl of Formula B1, C1, D1 or E1, one or more of R^{aA}, R⁵, R^{5A}, R⁶, R^{6A}, R⁷, R^{7A}, R⁸, R^{8A}, R⁹ and R^{9A} is —C(O)OH or —C(O)OR^{u} and in certain instances at least R⁷ is —C(O)OH or —C(O)OR^{u}, where R^{u} is an optionally substituted C1-C4 alkyl, butyl, tert-butyl, iso-butyl, propyl, iso-propyl, ethyl or methyl. In certain embodiments, one or more of R^{aA}, R⁵, R^{5A}, R⁶, R^{6A}, R⁷, R^{7A}, R⁸, R^{8A}, R⁹ and R^{9A} is —C(O)OH and in certain instances at least R⁷ is —C(O)OH.

In certain embodiments for which Y contains an optionally substituted aryl, optionally substituted heteroaryl, optionally substituted cycloalkyl or optionally substituted heterocycloalkyl of Formula B1, C1, D1 or E1, one or more of R^{aA}, R⁵, R^{5A}, R⁶, R^{6A}, R⁷, R^{7A}, R⁸, R^{8A}, R⁹ and R^{9A} is an optionally substituted C1-C4 haloalkyl, or a trifluoromethyl. In certain instances, one or two of R⁶, R⁷ and R⁸ each is, or R⁸ is, an optionally substituted C1-C4 haloalkyl, or a trifluoromethyl.

In certain embodiments for which Y contains an optionally substituted aryl, optionally substituted heteroaryl, optionally substituted cycloalkyl or optionally substituted heterocycloalkyl of Formula B1, C1, D1 or E1, one or more of R^{aA}, R⁵, R^{5A}, R⁶, R^{6A}, R⁷, R^{7A}, R⁸, R^{8A}, R⁹ and R^{9A} is an optionally substituted C1-C4 hydroxyalkyl or hydroxymethyleneyl. In certain instances, one or two of R⁶, R⁷ and R⁸ each is an optionally substituted C1-C4 hydroxyalkyl or hydroxymethyleneyl.

In certain embodiments, one of R^{a}, R⁵, R^{5A}, R⁶, R^{6A}, R⁷, R^{7A}, R⁸, R^{8A}, R⁹ and R^{9A} is a substituted heterocycloalkyl, substituted heterocycloalkylalkyl, unsubstituted heterocycloalkyl or unsubstituted heterocycloalkylalkyl. In certain instances, one of R^{aA}, R⁵, R^{5A}, R⁶, R^{6A}, R⁷, R^{7A}, R⁸, R^{8A}, R⁹ and R^{9A} is a substituted heterocycloalkylalkyl containing 5 or 6 ring atoms. In certain instances, one of R^{aA}, R⁵, R^{5A}, R⁶, R^{6A}, R⁷, R^{7A}, R⁸, R^{8A}, R⁹ and R^{9A} is a substituted heterocycloalkylalkyl containing 6 ring atoms and the alkyl attached to the heterocycloalkyl group is an unsubstituted C1-C4 alkylene, ethylene or methylene. In certain instances, one of R^{aA}, R⁵, R^{5A}, R⁶, R^{6A}, R⁷, R^{7A}, R⁸, R^{8A}, R⁹ and R^{9A} contains a substituted piperazinyl or piperidinyl and the alkyl attached to the heterocycloalkyl group is an unsubstituted ethylene or methylene. In certain instances, one of R^{aA}, R⁵, R^{5A}, R⁶, R^{6A}, R⁷, R^{7A}, R⁸, R^{8A}, R⁹ and R^{9A} is, or R⁷ is:

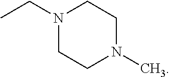

In certain embodiments, one of R^{a}, R⁵, R^{5A}, R⁶, R^{6A}, R⁷, R^{7A}, R⁸, R^{8A}, R⁹ and R^{9A} is a substituted heteroaryl, substituted heteroarylalkyl, unsubstituted heteroaryl or unsubstituted heteroarylalkyl. In certain instances, one of R^{aA}, R⁵, R^{5A}, R⁶, R^{6A}, R⁷, R^{7A}, R⁸, R^{8A}, R⁹ and R^{9A} is a substituted heteroaryl or unsubstituted heteroaryl each containing 5 or 6 ring atoms. In certain instances, one of R^{aA}, R⁵, R^{5A}, R⁶, R^{6A}, R⁷, R^{7A}, R⁸, R^{8A}, R⁹ and R^{9A} is a substituted heteroaryl containing 5 ring atoms. In certain instances, one of R^{aA}, R⁵, R^{5A}, R⁶, R^{6A}, R⁷, R^{7A}, R⁸, R^{8A}, R⁹ and R^{9A} is a substituted pyrrolyl, substituted imidazolyl, or substituted pyrazolyl. In certain instances, one of R^{aA}, R⁵, R^{5A}, R⁶, R^{6A}, R⁷, R^{7A}, R⁸, R^{8A}, R⁹ and R^{9A} is, or R⁶ is:

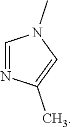

In certain embodiments pertaining to a compound of Formula A1-1, Formula A1-2, Formula A1-5, Formula A1-6, Formula A1-8, Formula A2-1 or Formula A2-3, one, two, three or four of R⁵, R⁶, R⁸ and R⁹ each is hydrogen.

In certain embodiments for which Y contains an optionally substituted aryl, optionally substituted heteroaryl, optionally substituted cycloalkyl or optionally substituted heterocycloalkyl of Formula B1, C1, D1 or E1, one or more of R⁵, R^{5A}, R⁶, R^{6A}, R⁷, R^{7A}, R⁸, R^{8A}, R⁹ and R^{9A} is a Formula F group defined for Formula A1 and Formula A2. In certain embodiments, R⁷ is a Formula F group defined for Formula A1 and Formula A2.

In certain embodiments, Y is a Formula F group defined for a compound of Formula A1 and Formula A2. In certain embodiments, Y is —N(R^{b})—CH₂—R^{Y} and R^{Y} is a Formula F group defined for Formula A1 and Formula A2. In certain embodiments, Y is —N(R^{a})R^{b}, R^{a} is a Formula F group and R^{b} is hydrogen, optionally substituted C1-C6 alkyl, optionally substituted C1-C4 alkyl, butyl, tert-butyl, iso-butyl, propyl, iso-propyl, ethyl or methyl. In certain embodiments, Y is —N(R^{b})C(O)—R^{V}—R^{Y} and R^{Y} is a Formula F group defined for Formula A1 and Formula A2. In certain instances, R^{V} is an optionally substituted C1-C4 alkylene, —CH₂CH₂— or —CH₂—. In certain instances, R^{b} is hydrogen, optionally substituted C1-C4 alkyl, butyl, tert-butyl, iso-butyl, propyl, iso-propyl, ethyl or methyl.

In certain embodiments pertaining to a compound that contains a Formula F group:
Z¹ is an optionally substituted piperidinyl, optionally substituted piperazinyl or optionally substituted morpholinyl;
Z¹ is an unsubstituted piperidinyl, unsubstituted piperazinyl or unsubstituted morpholinyl;
Z¹ is a substituted piperidinyl, substituted piperazinyl or substituted morpholinyl containing one or more C1-C6 alkyl substituents, or containing one or more C1-C4 alkyl substituents, or containing one or more ethyl or methyl substituents, or containing one methyl substituent;
X^{a} is C(R⁴³)R^{43A}; X^{b} is C(R⁴⁴)R^{44A}; X^{c} is N—R^{45B}; X^{d} is C(R⁴⁶)R^{46A}; and X^{e} is C(R⁴⁷)R^{47A};
X^{a} is C(R⁴³)R^{43A}; X^{b} is C(R⁴⁴)R^{44A}; X^{c} is O; X^{d} is C(R⁴⁶)R^{46A}; and X^{e} is C(R⁴⁷)R^{47A};
X^{a} is C(R⁴³)R^{43A}; X^{b} is C(R⁴⁴)R^{44A}; X^{c} is C(R⁴⁵)R^{45A}; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A} and R^{47A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{45B} is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl;
R⁴³, R⁴⁴, R⁴⁵, R⁴⁶, R⁴⁷, R^{43A}, R^{44A}, R^{45A}, R^{46A} and R^{47A} each independently is hydrogen, unsubstituted C1-C4 alkyl, Cl, F, CF₃, CD₃, unsubstituted C1-C4 alkoxy, methoxy, isopropyloxy or dimethylamino;
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C4 alkyl, ethyl or methyl;
R⁴³, R⁴⁴, R⁴⁵, R⁴⁶, R⁴⁷, R^{43A}, R^{44A}, R^{46A} and R^{47A} each is hydrogen and R^{45A} and R^{45B} each is hydrogen or optionally substituted C1-C6 alkyl, optionally substituted C1-C4 alkyl, ethyl or methyl;
R⁴³, R⁴⁴, R⁴⁵, R⁴⁶, R⁴⁷, R^{43A}, R^{44A}, R^{46A} and R^{47A} each is hydrogen and R^{45A} and R^{45B} is methyl; and/or
the Formula F group is:

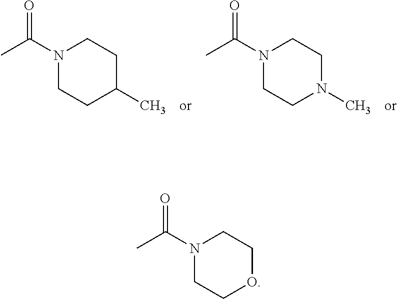

In certain embodiments, Y is —N(R^{b})C(O)—R^{Y} or —N(R^{b})C(O)—R^{V}—R^{Y} and R^{Y} is hydrogen, optionally substituted alkyl, optionally substituted alkynyl, optionally substituted alkoxy, optionally substituted heteroalkyl, optionally substituted haloalkyl, optionally substituted deuteroalkyl, optionally substituted hydroxyalkyl, optionally substituted mercaptoalkyl or optionally substituted aminoalkyl, and R^{V} and R^{b} are as defined for Formula A1 and Formula A2. In certain instances, R^{V} is an optionally substituted C1-C4 alkylene, —CH₂CH₂— or —CH₂—. In certain instances, R^{b} is hydrogen or optionally substituted C1-C4 alkyl, ethyl or methyl. In certain instances, R^{Y} is an optionally substituted C1-C6 alkyl, optionally substituted C1-C4 alkyl, butyl, tert-butyl, iso-butyl, propyl, iso-propyl, ethyl or methyl. In certain instances, R^{Y} is an optionally substituted C1-C6 alkynyl, optionally substituted C1-C4 alkynyl or —C≡CH.

In certain embodiments, Y is —N(R^{b})C(O)—R^{Y} or —N(R^{b})C(O)—R^{V}—R^{Y}, R^{Y} is an optionally substituted alkenyl, and R^{V} and R^{b} are as defined for Formula A1 and Formula A2. In certain instances R^{Y} is —CH═CH₂, and optionally is subject to a limitation defined herein.

In certain embodiments, Y is —N(R^{a})C(O)—R^{V}—N(R^{b})R^{Y}; R^{a}, R^{b} and R^{V} are as defined for Formula A1 and Formula A2, and R^{Y} is hydrogen or optionally substituted alkyl. In certain instances, R^{Y} is hydrogen, substituted C1-C4 alkyl, unsubstituted C1-C4 alkyl, butyl, tert-butyl, iso-butyl, propyl, iso-propyl, ethyl or methyl. In certain instances, R^{V} is an optionally substituted C1-C4 alkylene, optionally substituted C1-C3 alkylene, —CH₂CH₂— or —CH₂—. In certain instances, R^{b} is hydrogen or optionally substituted C1-C4 alkyl, butyl, tert-butyl, iso-butyl, propyl, iso-propyl, ethyl or methyl.

In certain embodiments, Y is —N(R^{a})R^{b} and R^{a} and R^{b} each is hydrogen.

R¹⁰, R¹¹, R¹² and R¹³ Substituents

In certain embodiments, at least one of, or one of, R¹⁰, R¹¹, R¹² and R¹³ is R^{W} or —W—R^{W}, and R^{W} is as defined for Formula A1 and Formula A2. In certain embodiments, R¹⁰ is R^{W} or —W—R^{W} and R¹¹, R¹² and R¹³ each is not R^{W} or —W—R^{W}; or R¹¹ is R^{W} or —W—R^{W} and R¹⁰, R¹² and R¹³ each is not R^{W} or —W—R^{W}; or R¹² is R^{W} or —W—R^{W} and R¹⁰, R¹¹ and R¹³ each is not R^{W} or —W—R^{W}; or R¹³ is R^{W} or —W—R^{W} and R¹⁰, R¹¹ and R¹² each is not R^{W} or —W—R^{W}. In certain instances, none of R¹⁰, R¹¹, R¹² and R¹³ is R^{W} or —W—R^{W}.

For embodiments in which R¹⁰, R¹¹, R¹² or R¹³ is not R^{W} or —W—R^{W}, the R¹⁰, R¹¹, R¹² and R¹³ that is not R^{W} or —W—R^{W} each independently can be hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino or amido.

For embodiments in which R¹⁰, R¹¹, R¹² or R¹³ is not R^{W} or —W—R^{W}, the R¹⁰, R¹¹, R¹² and R¹³ that is not R^{W} or —W—R^{W} each independently can be hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, hydroxy, halo, cyano or nitro; and R^{g}, R^{h}, R^{j} and R^{k} each independently is hydrogen or optionally substituted C1-C6 alkyl. In certain instances, R^{g}, R^{h}, R^{j} and R^{k} each independently is hydrogen, optionally substituted C1-C4 alkyl, butyl, iso-butyl, tert-butyl, propyl, isopropyl, ethyl or methyl. In certain embodiments, R^{u} is an optionally substituted C1-C4 alkyl, butyl, iso-butyl, tert-butyl, propyl, isopropyl, ethyl or methyl.

For embodiments in which R¹⁰, R¹¹, R¹² or R¹³ is not R^{W} or —W—R^{W}, the R¹⁰, R¹¹, R¹² and R¹³ that is not R^{W} or —W—R^{W} each independently can be hydrogen, optionally substituted C1-C4 alkyl, optionally substituted C1-C4 alkoxy, ethyl, ethoxy, methyl, methoxy, chloro, fluoro, bromo, iodo, CF₃ or CD₃. In certain instances, one of R¹⁰, R¹¹, R¹² and R¹³ is R^{W} or —W—R^{W}, and one, two or three of R¹⁰, R¹¹, R¹² and R¹³ that is not R^{W} or —W—R^{W} each is hydrogen.

In certain embodiments, W is —CH₂—, —C≡C—, —NH(R^{t})—, —O—, —S—, —S(O)— or —SO₂—, and R^{t} is hydrogen or optionally substituted C1-C6 alkyl, optionally substituted C1-C4 alkyl, ethyl or methyl, and R^{W} is as defined herein for a compound of Formula A1 and Formula A2.

In certain embodiments, at least one of, or one of, R¹⁰, R¹¹, R¹² and R¹³ is R^{W} or —W—R^{W}, and R^{W} contains or is an optionally substituted aryl, optionally substituted arylalkyl, optionally substituted heteroaryl or optionally substituted heteroarylalkyl. In certain instances, an optionally substituted arylalkyl comprises an optionally substituted alkylene (for example, an optionally substituted C1-C6 alkylene), linked to an optionally substituted aryl. In certain embodiments, an optionally substituted heteroarylalkyl comprises an optionally substituted alkylene (for example, an optionally substituted C1-C6 alkylene), linked to an optionally substituted heteroaryl. In certain instances, R^{W} contains an optionally substituted aryl or optionally substituted heteroaryl according to Formula B2:

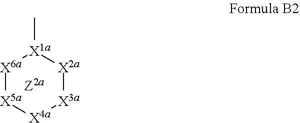

where: Z^{2a} is aryl or heteroaryl; X^{1a} independently is C or N; X^{2a} independently is C—R¹⁴, N—R^{14B} or N; X^{3a} independently is C—R¹⁵, N—R^{15B} or N; X^{4a} independently is C—R¹⁶, N—R^{16B} or N; X^{5a} independently is C—R¹⁷, N—R^{17B} or N; and X^{6a} independently is C—R¹⁸, N—R^{18B} or N; and optionally two adjacent R¹⁴, R¹⁵, R¹⁶, R¹⁷, R¹⁸, R^{14B}, R^{15B}, R^{16B}, R^{17B} and R^{18B} are linked in an optionally substituted aryl or optionally substituted heteroaryl.

In certain embodiments pertaining to Formula B2, Z^{2a} is an optionally substituted pyridinyl, optionally substituted pyrimidinyl, optionally substituted pyrazinyl, optionally substituted pyridazinyl or optionally substituted triazinyl. In certain embodiments pertaining to Formula B2, X^{1a} is C, X^{2a} is C—R¹⁴, X^{3a} is C—R¹⁵, X^{4a} is C—R¹⁶, X^{5a} is C—R¹⁷ and X^{6a} is C—R¹⁸. In certain embodiments pertaining to Formula B2, X^{1a} is C, X^{2a} is C—R¹⁴, X^{3a} is N, X^{4a} is C—R¹⁶, X^{5a} is C—R¹⁷, and X^{6a} is C—R¹⁸. In certain embodiments pertaining to Formula B2, X^{1a} is C, X^{2a} is N, X^{3a} is C—R¹⁵, X^{4a} is N, X^{5a} is C—R¹⁷, and X^{6a} is C—R¹⁸. In certain embodiments pertaining to Formula B2, X^{1a} is C, X^{2a} is C—R¹⁴, X^{3a} is N, X^{4a} is C—R¹⁶, X^{5a} is N, and X^{6a} is C—R¹⁸. In certain embodiments pertaining to Formula B2, X^{1a} is C, X^{2a} is C—R¹⁴, X^{3a} is C—R¹⁵, X^{4a} is N, X^{5a} is C—R¹⁷ and X^{6a} is C—R¹⁸.

In certain embodiments pertaining to Formula B2, X^{1a} is C, X^{2a} is C—R¹⁴, X^{3a} is C—R¹⁵, X^{4a} is C—R¹⁶, X^{5a} is C—R¹⁷ and X^{6a} is C—R¹⁸, and R¹⁶ and R¹⁷ together are joined as a fused optionally substituted aryl, optionally substituted heteroaryl, optionally substituted cycloalkyl or optionally substituted heterocycloalkyl containing five ring member atoms. In certain embodiments pertaining to Formula B2, X^{1a} is C, X^{2a} is C—R¹⁴, X^{3a} is N, X^{4a} is C—R¹⁶, X^{5a} is C—R¹⁷, and X^{6a} is C—R¹⁸, where the X^{3a} nitrogen and R¹⁶ together are joined as a fused optionally substituted aryl or optionally substituted heteroaryl containing five ring member atoms. In certain instances, the fused ring is an optionally substituted heteroaryl containing five ring atoms, and in certain instances, the fused ring is an optionally substituted pyrrolyl or oxazolyl. In certain embodiments, R^{W} is an optionally substituted benzo-oxazolyl group or an optionally substituted benzo[d]oxazol-5-yl group. In certain instances, R^{W} is an optionally substituted indolyl group or optionally substituted 1H-indol-5-yl group. In certain instances, R^{W} is an optionally substituted imidazo-pyridinyl group or an optionally substituted imidazo[1,2-a]pyridin-6-yl group. In certain instances, a compound includes a R^{W} substituent of Cmpd 34, Cmpd 38 or Cmpd 40 illustrated in Table A.

In certain embodiments, at least one of, or one of, R¹⁰, R¹¹, R¹² and R¹³ is R^{W} or —W—R^{W}, and R^{W} contains or is an optionally substituted aryl or optionally substituted heteroaryl according to Formula B3:

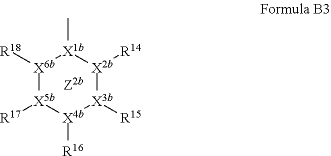

where: Z^{2b} is aryl or heteroaryl; X^{1b}, X^{2b}, X^{3b}, X^{4b}, X^{5b} and X^{6b} each independently is C or N; R¹⁴, R¹⁵, R¹⁶, R¹⁷, and R¹⁸ each are as defined herein; and optionally two adjacent R¹⁴, R¹⁵, R¹⁶, R¹⁷, and R¹⁸ are linked in an optionally substituted aryl or optionally substituted heteroaryl. In certain embodiments pertaining to Formula B3, one of X^{1b}, X^{2b}, X^{3b}, X^{4b}, X^{5b}, and X^{6b} is N; or two of X^{1b}, X^{2b}, X^{3b}, X^{4b}, X^{5b} and X^{6b} are N; or three of X^{1b}, X^{2b}, X^{3b}, X^{4b}, X^{5b} and X^{6b} are N; or four of X^{1b}, X^{2b}, X^{3b}, X^{4b}, X^{5b} and X^{6b} are N. In certain instances pertaining to Formula B3, Z^{2b} is aryl and each of X^{1b}, X^{2b}, X^{3b}, X^{4b}, X^{5b} and X^{6b} is C.

In certain embodiments, at least one of, or one of, R¹⁰, R¹¹, R¹² and R¹³ is R^{W} or —W—R^{W}, and R^{W} contains or is an optionally substituted aryl or optionally substituted heteroaryl according to Formula C2:

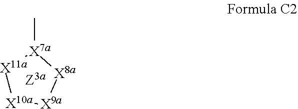

where: Z^{3a} is aryl or heteroaryl; X^{7a} independently is C or N; X^{8a} independently is C—R¹⁹, N—R^{19B}, N, O, S, S(O) or SO₂; X^{9a} independently is C—R²⁰, N—R^{20B}, N, O, S, S(O) or SO₂; X^{10a} independently is C—R²¹, N—R^{21B}, N, O, S, S(O) or SO₂; and X^{11a} independently is C—R²², N—R^{22B}, N, O, S, S(O) or SO₂; and optionally two adjacent R¹⁹, R²⁰, R²¹, R²², R^{19B}, R^{20B}, R^{21B} and R^{22B} are linked in an optionally substituted aryl or optionally substituted heteroaryl.

In certain embodiments pertaining to Formula C2, Z^{3a} is an optionally substituted pyrrolyl, optionally substituted imidazolyl, optionally substituted pyrazolyl, optionally substituted puranyl, optionally substituted thiophenyl, optionally substituted oxazolyl, optionally substituted thiazolyl, optionally substituted isoxazolyl or optionally substituted isothiazolyl. In certain embodiments pertaining to Formula C2, X^{7a} is C; X^{8a} is N or N—R^{19B}; X^{9a} is N or N—R^{20B}; X^{10a} is C—R²¹; and X^{11a} is C—R²². In certain embodiments pertaining to Formula C2, X^{7a} is C; X^{8a} is N or N—R^{19B}; X^{9a} is C—R²⁰; X^{10a} is C—R²¹; and X^{11a} is C—R²². In certain embodiments pertaining to Formula C2, X^{7a} is C; X^{8a} is N or N—R^{19B}; X^{9a} is C—R²⁰; X^{10a} is C—R²¹; and X^{11a} is N or N—R^{22B}. In certain embodiments pertaining to Formula C2, X^{7a} is C; X^{8a} is N; X^{9a} is C—R²⁰; X^{10a} is C—R²¹; and X^{11a} is S. In certain embodiments pertaining to Formula C2, X^{7a} is C; X^{8a} is N; X^{9a} is C—R²⁰; X^{10a} is O; and X^{11a} is C—R²².

In certain embodiments pertaining to Formula C2, X^{7a} is C; X^{8a} is N or N—R^{19B}; X^{9a} is C—R²⁰; X^{10a} is C—R²¹; and X^{11a} is C—R²² and R²⁰ and R²¹ together are joined as a fused optionally substituted aryl, optionally substituted heteroaryl, optionally substituted cycloalkyl or optionally substituted heterocycloalkyl containing six ring member atoms. In certain instances, the fused ring is an optionally substituted heteroaryl containing five ring atoms, and in certain instances, the fused ring is an optionally substituted aryl or heteroaryl containing six ring atoms. In certain instances, the fused ring is an optionally substituted phenyl. In certain embodiments, R^{W} is an optionally substituted indolyl group or an optionally substituted 1H-indol-2-yl group. In certain instances, a compound includes a R^{W} substituent of Cmpd 33 illustrated in Table A.

In certain embodiments, at least one of, or one of, R¹⁰, R¹¹, R¹² and R¹³ is R^{W} or —W—R^{W}, and R^{W} contains or is an optionally substituted aryl or optionally substituted heteroaryl according to Formula C3:

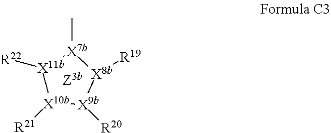

where: Z^{3b} is aryl or heteroaryl; X^{7b}, X^{8b}, X^{9b}, X^{10b} and X^{11b} each independently is C, N, O or S; R¹⁹, R²⁰, R²¹ and R²² each is as defined herein; and optionally two adjacent R¹⁹, R²⁰, R²¹ and R²² are linked in an optionally substituted aryl or optionally substituted heteroaryl. In Formula C3, each of R¹⁹, R²⁰, R²¹ and R²² optionally is present and presence or absence is determined by the associated ring member atom X^{8b}, X^{9b}, X¹⁰⁰ and X^{11b}. For embodiments in which ring member atom X^{8b}, X^{9b}, X^{10b} or X^{11b} is C or N, the R group shown in Formula C3 as bonded to the ring member atom is present or optionally present or not present. For example, where X^{8b} is C or N, R¹⁹ can be present; where X^{9b} is C or N, R²⁰ can be present; where X^{10b} is C or N, R²¹ can be present; and where X^{11b} is C or N, R²² can be present. In certain embodiments, one of X^{7b}, X^{8b}, X^{9b}, X^{10b} and X^{11b} is N; or two of X^{7b}, X^{8b}, X^{9b}, X^{10b} and X^{11b} are N; or three of X^{7b}, X^{8b}, X^{9b}, X^{10b} and X^{11b} are N; or four of X^{7b}, X^{8b}, X^{9b}, X^{10b} and X^{11b} are N. In certain embodiments, one of X^{7b}, X^{8b}, X^{9b}, X^{10b} and X^{11b} is N and the others are C; or two of X^{7b}, X^{8b}, X^{9b}, X^{11b} and X^{11b} are N and the others are C; or three of X^{7b}, X^{8b}, X^{9b}, X^{10b} and X^{11b} are N and the others are C; or four of X^{7b}, X^{8b}, X^{9b}, X^{11b} and X^{11b} are N and the other is C. For embodiments in which ring member atom X^{8b}, X^{9b}, X^{10b} or X^{11b} is O or S, the R group shown in Formula C3 as bonded to the ring member atom can be absent. For example, where X^{8b} is O or S, R¹⁹ can be absent; where X^{9b} is O or S, R²⁰ can be absent; where X^{11b} is O or S, R^{21b} can be absent; and where X^{11b} is O or S, R²² can be absent. For embodiments in which ring member atom X^{8b}, X^{9b}, X^{10b} or X^{11b} is S, the S may be in a reduced state (S) or an oxidized state (S(O) or SO₂). In certain embodiments, one of X^{8b}, X^{9b}, X^{10b} and X^{11b} is O or S; or two of X^{8b}, X^{9b}, X^{10b} and X^{11b} are O or S; or three of X^{8b}, X^{9b}, X^{10b} and X^{11b} are O or S. In certain embodiments, one of X^{8b}, X^{9b}, X^{10b} and X^{11b} is O or S and the others are C; or two of X^{8b}, X^{9b}, X^{10b} and X^{11b} are O or S and the others are C; or three of X^{8b}, X^{9b}, X^{10b} and X^{11b} are O or S and the other is C. In certain embodiments, zero, one or two of X^{7b}, X^{8b}, X^{9b}, X^{10b} and X^{11b} is N and X^{7b}, X^{8b}, X^{9b}, X^{10b} and X^{11b} that are not N are C. In certain instances, Z^{3b} is heteroaryl; X^{7b}, X^{10b} and X^{11b} each is C, and one of X^{8b} and X^{9b} is N and the other is C. In certain embodiments, X^{8b} and X^{9b} each is N.

In certain embodiments, at least one of, or one of, R¹⁰, R¹¹, R¹² and R¹³ is R^{W} or —W—R^{W}, and R^{W} contains or is an optionally substituted cycloalkyl, optionally substituted cycloalkylalkyl, optionally substituted heterocycloalkyl or an optionally substituted heterocycloalkylalkyl. In certain embodiments, R^{W} is an optionally substituted cycloalkylalkyl comprising an optionally substituted alkylene, for example an optionally substituted C1-C6 alkylene, linked to an optionally substituted cycloalkyl. In certain embodiments, R^{W} is an optionally substituted heterocycloalkylalkyl comprising an optionally substituted alkylene, for example an optionally substituted C1-C6 alkylene, linked to an optionally substituted heterocycloalkyl. In certain embodiments, R^{W} contains an optionally substituted cycloalkyl or optionally substituted heterocycloalkyl according to Formula D2:

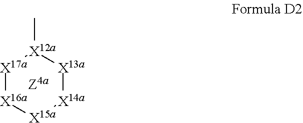

where: Z^{4a} is cycloalkyl or heterocycloalkyl; X^{12a} is C—R^{aB}, C or N; X^{13a} independently is C(R¹⁴)R^{14A}, C—R¹⁴, N—R^{14B}, N, O, S, S(O) or SO₂; X^{14a} independently is C(R¹⁵)R^{15A}, C—R¹⁵, N—R^{15B}, N, O, S, S(O) or SO₂; X^{15a} independently is C(R¹⁶)R^{16A}, C—R¹⁶, N—R^{16B}, N, O, S, S(O) or SO₂; X^{16a} independently is C(R¹⁷)R^{17A}, C—R¹⁷, N—R^{17B}, N, O, S, S(O) or SO₂; and X^{17a} independently is C(R¹⁸)R^{18A}, C—R¹⁸, N—R^{18B}, N, O, S, S(O) or SO₂; R¹⁴, R¹⁵, R¹⁶, R¹⁷, R¹⁸, R^{14A}, R^{15A}, R^{16A}, R^{17A}, R^{18A}, R^{14B}, R^{15B}, R^{16B}, R^{17B} and R^{18B} are as defined herein; and optionally two adjacent R¹⁴, R¹⁵, R¹⁶, R¹⁷, R¹⁸, R^{14A}, R^{15A}, R^{16A}, R^{17A}, R^{18A}, R^{14B}, R^{15B}, R^{16B}, R^{17B} and R^{18B} are linked and joined in an optionally substituted cycloalkyl or optionally substituted heterocycloalkyl. In certain embodiments, R^{W} is an optionally substituted heterocycloalkyl or an optionally substituted heterocycloalkylalkyl of which the optionally substituted heterocycloalkyl contains 4, 5 or 6 ring atoms. In certain instances, R^{W} contains an optionally substituted piperidinyl, optionally substituted piperazinyl, optionally substituted tetrahydropyran, or optionally substituted morpholinyl, optionally substituted pyrrolidinyl, optionally substituted imidazolidinyl, optionally substituted pyrazolidinyl or optionally substituted azetidinyl. In certain instances, R^{W} contains a substituted heterocycloalkyl containing 4, 5 or 6 ring atoms, which sometimes is a C1-C6 alkyl substituted heterocycloalkyl, a methyl substituted heterocycloalkyl or a mono-methyl substituted heterocycloalkyl.

In certain instances pertaining to Formula D2, Z^{4a} is an optionally substituted piperidinyl, optionally substituted piperazinyl, optionally substituted morpholinyl or optionally substituted tetrahydropyridinyl. In certain instances pertaining to Formula D2, X^{12a} is C; X^{13a} is C—R¹⁴; X^{14a} is C(R¹⁵)R^{15A}; X^{15a} is N—R^{16B}; X^{16a} is C(R¹⁷)R^{17A}; and X^{17a} is C(R¹⁸)R^{18A}. In certain instances pertaining to Formula D2, X^{12a} is N; X^{13a} is C(R¹⁴)R^{14A}; X^{14a} is C(R¹⁵)R^{15A}; X^{15a} is N—R^{16B}; X^{16a} is C(R¹⁷)R^{17A}; and X^{17a} is C(R¹⁸)R^{18A}. In certain instances pertaining to Formula D2, X^{12a} is N; X^{13a} is C(R¹⁴)R^{14A}; X^{14a} is C(R¹⁵)R^{15A}; X^{15a} is C(R¹⁶)R^{16A}; X^{16a} is C(R¹⁷)R^{17A}; and X^{17a} is C(R¹⁸)R^{18A}. In certain embodiments pertaining to Formula D2, X^{12a} is C—R^{aB}; X^{13a} is C(R¹⁴)R^{14A}; X^{14a} is C(R¹⁵)R^{15A}; X^{15a} is N—R^{16B}; X^{16a} is C(R¹⁷)R^{17A} and X^{17a} is C(R¹⁸)R^{18A}. In certain instances pertaining to Formula D2, X^{12a} is C—R^{aB}; X^{13a} is C(R¹⁴)R^{14A}; X^{14a} is C(R¹⁵)R^{15A}; X^{15a} is O; X^{16a} is C(R¹⁷)R^{17A}; and X^{17a} is C(R¹⁸)R^{18A}.

In certain embodiments, at least one of, or one of, R¹⁰, R¹¹, R¹² and R¹³ is R^{W} or —W—R^{W}, and R^{W} contains or is an optionally substituted cycloalkyl or optionally substituted heterocycloalkyl according to Formula D3:

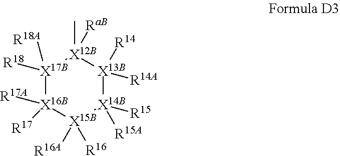

where: Z^{4b} is cycloalkyl or heterocycloalkyl; X^{12b} is C or N; X^{13b}, X^{14b}, X^{15b}, X^{16b} and X^{17b} each independently is C, N, O or S; R¹⁴, R¹⁵, R¹⁶, R¹⁷, R¹⁸, R^{14A}, R^{15A}, R^{16A}, R^{17A} and R^{18A} are as defined herein; and optionally and optionally two adjacent R¹⁴, R¹⁵, R¹⁶, R¹⁷, R¹⁸, R^{14A}, R^{15A}, R^{16A}, R^{17A} and R^{18A} are linked and joined in an optionally substituted cycloalkyl or optionally substituted heterocycloalkyl. In Formula D3, each of R^{aB}, R¹⁴, R¹⁵, R¹⁶, R¹⁷, R¹⁸, R^{14A}, R^{15A}, R^{16A}, R^{17A} and R^{18A} is an optionally present and presence or absence is determined by the associated ring member atom X^{12b}, X^{13b}, X^{14b}, X^{15b}, X^{16b} and X^{17b}. For embodiments in which ring member atom X^{12b}, X^{13b}, X^{14b}, X^{15b}, X^{16b} and X^{17b} is C, R groups shown in Formula D3 as bonded to the ring member atom may be present (e.g., both are present when ring Z^{4b} is a saturated ring). For example, where X^{12b} is C, R^{aB} may be present; where X^{13b} is C, R¹⁴ and R^{14A} may be present; where X^{14b} is C, R¹⁵ and R^{15A} may be present; where X^{15b} is C, R¹⁶ and R^{16A} may be present; where X^{16b} is C, R¹⁷ and R^{17A} may be present; or where X^{17b} is C, R¹⁸ and R^{18A} may be present. For embodiments in which ring member atom X^{12b}, X^{13b}, X^{14b}, X^{15b}, X^{16b} or X^{17b} is N, an R group shown in Formula D3 as bonded to the ring member atom may be absent. For example, where X^{12b} is N, R^{aB} may be absent; X¹³¹ is N, R¹⁴ or R^{14A} may be absent; where X^{14b} is N, R¹⁵ or R^{15A} may be absent; where X^{15b} is N, R¹⁶ or R^{16A} may be absent; where X^{16b} is N, R¹⁷ or R^{17A} may be absent; or where X^{17b} is N, R¹⁸ or R^{18A} may be absent. In certain embodiments, one of X^{12b}, X^{13b}, X^{14b}, X^{15b}, X^{16b} and X^{17b} is N; or two of X^{12b}, X^{13b}, X^{14b}, X^{15b}, X^{16b} and X^{17b} are N; or three of X^{12b}, X^{13b}, X^{14b}, X^{15b}, X^{16b} and X^{17b} are N; or four of X^{12b}, X^{13b}, X^{14b}, X^{15b}, X^{16b} and X^{17b} are N. In certain embodiments, one of X^{12b}, X^{13b}, X^{14b}, X^{15b}, X^{16b} and X^{17b} is N and the others are C; or two of X^{12b}, X^{13b}, X^{14b}, X^{15b}, X^{16b} and X^{17b} are N and the others are C; or three of X^{12b}, X^{13b}, X^{14b}, X^{15b}, X^{16b} and X^{17b} are N and the others are C; or four of X^{12b}, X^{13b}, X^{14b}, X^{15b}, X^{16b} and X^{17b} are N and the others are C. For embodiments in which ring member atom X^{13b}, X^{14b}, X^{15b}, X^{16b} or X^{17b} is O or S, both R groups shown in Formula D3 as bonded to the ring member atom may be absent. For example, where X^{13b} is O or S, R¹⁴ and R^{14A} may be absent; where X^{14b} is O or S, R¹⁵ and R^{15A} may be absent; where X^{15b} is O or S, R¹⁶ and R^{16A} may be absent; where X^{16b} is O or S, R¹⁷ and R^{17A} may be absent; and where X^{17b} is O or S, R¹⁸ and R^{18A} may be absent. For embodiments in which ring member atom X^{13b}, X^{14b}, X^{15b}, X^{16b} and X^{17b} is S, the S may be in a reduced state (S) or an oxidized state (S(O) or SO₂). In certain embodiments, one of X^{13b}, X^{14b}, X^{15b}, X^{16b} and X^{17b} is O or S; or two of X^{13b}, X^{14b}, X^{15b}, X^{16b} and X^{17b} are or S; or three of X^{13b}, X^{14b}, X^{15b}, X^{16b} and X^{17b} are O or S; or four of X^{13b}, X^{14b}, X^{15b}, X^{16b} and X^{17b} are O or S. In certain embodiments, one of X^{13b}, X^{14b}, X^{15b}, X^{16b} and X^{17b} is O or S and the others are C; or two of X^{13b}, X^{14b}, X^{15b}, X^{16b} and X^{17b} are O or S and the others are C; or three of X^{13b}, X^{14b}, X^{15b}, X^{16b} and X^{17b} are O or S and the others are C; or four of X^{13b}, X^{14b}, X^{15b}, X^{16b} and X^{17b} are O or S and the others are C. In certain embodiments, zero, one or two of X^{12b}, X^{13b}, X^{14b}, X^{15b}, X^{16b} and X^{17b} in Formula D3 is N and X^{12b}, X^{13b}, X^{14b}, X^{15b}, X^{16b} and X^{17b} that are not N are C. In certain instances, Z³ is cycloalkyl and each of X^{12b}, X^{13b}, X^{14b}, X^{15b}, X^{16b} and X^{17b} is C.

In certain embodiments, at least one of, or one of, R¹⁰, R¹¹, R¹² and R¹³ is R^{W} or —W—R^{W}, and R^{W} contains or is an optionally substituted cycloalkyl or optionally substituted heterocycloalkyl of Formula E2:

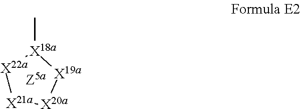

where: Z^{5a} is cycloalkyl or heterocycloalkyl; X^{18a} is C—R^{aB}, C or N; X^{19a} independently is C(R¹⁹)R^{19A}, C—R¹⁹, N—R^{19B}, N, O, S, S(O) or SO₂; X^{20a} independently is C(R²⁰)R^{20A}, C—R²⁰, N—R^{20B}, N, O, S, S(O) or SO₂; X^{21a} independently is C(R²¹)R^{21A}, C—R²¹, N—R^{21B}, N, O, S, S(O) or SO₂; and X^{22a} independently is C(R²²)R^{22A}, C—R²², N—R^{22B}, N, O, S, S(O) or SO₂; R^{aB}, R¹⁹, R^{19A}, R²⁰, R^{20A}, R²¹, R^{21A}, R²², R^{22A}, R^{19B}, R^{20B}, R^{21B} and R^{22B} are as defined herein; and optionally two adjacent R^{aB}, R¹⁹, R^{19A}, R²⁰, R²⁰, R²¹, R^{21A}, R²², R^{22A}, R^{19B}, R^{20B}, R^{21B} and R^{22B} are linked in an optionally substituted cycloalkyl or optionally substituted heterocycloalkyl. In certain embodiments pertaining to Formula E2, Z^{5a} is an optionally substituted pyrazolidinyl, optionally substituted imidazolidinyl or optionally substituted pyrazolidinyl. In certain embodiments pertaining to Formula E2, X^{18a} is C—R^{aB}; X^{19a} is N—R^{19B}; X^{20a} is C(R²⁰)R^{20A}; X^{21a} is C(R²¹)R^{21A}; and X^{22a} is C(R²²)R^{22A}.

In certain embodiments, at least one of, or one of, R¹⁰, R¹¹, R¹² and R¹¹ is R^{W} or —W—R^{W}, and R^{W} contains or is an optionally substituted cycloalkyl or optionally substituted heterocycloalkyl of Formula E3:

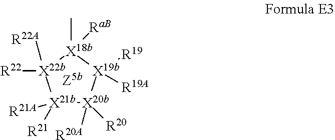

where: Z^{5b} is cycloalkyl or heterocycloalkyl; X^{18b} is C or N; X^{19b}, X^{20b}, X^{21b} and X^{22b} each independently is C, N, O or S; R^{aB}, R¹⁹, R^{19A}, R²⁰, R²⁰, R²¹, R^{21A}, R²² and R^{22A} are as defined herein; and optionally two adjacent R^{aB}, R¹⁹, R^{19A}, R²⁰, R^{20A}, R²¹, R^{21A}, R²² and R^{22A} are linked in an optionally substituted cycloalkyl or optionally substituted heterocycloalkyl. In Formula E3, each of R^{aB}, R¹⁹, R^{19A}, R²⁰, R^{20A}, R²¹, R^{21A}, R²² and R^{22A} optionally is present and presence or absence is determined by the associated ring member atom X^{18b}, X^{19b}, X^{20b}, X^{21b} and X^{22b}. For embodiments in which ring member atom X^{18b}, X^{19b}, X^{20b}, X^{21b} or X^{22b} is C, R groups shown in Formula E3 as bonded to the ring member atom may be present (e.g., both are present when ring Z^{5b} is a saturated ring). For example, where X^{18b} is C, R^{aB} may be present; where X^{19b} is C, R¹⁹ and R^{19A} may be present; where X^{20b} is C, R²⁰ and R^{20A} may be present; where X^{21b} is C, R²¹ and R^{21A} may be present; or where X^{22b} is N, R²² and R^{22A} may be present. For embodiments in which ring member atom X^{18b}, X^{19b}, X^{20b}, X^{21b} or X^{22b} is N, an R group shown in Formula E as bonded to the ring member atom may be absent. For example, where X^{18b} is N, R^{aB} may be absent; where X^{19b} is N, R¹⁹ or R^{19A} may be absent; where X^{20b} is N, R²⁰ or R^{20A} may be absent; where X^{21b} is N, R²¹ or R^{21A} may be absent; or where X^{22b} is N, R²² or R^{22A} may be absent. In certain embodiments, one of X^{18b}, X^{19b}, X^{20b}, X^{21b} and X^{22b} is N; or two of X^{18b}, X^{19b}, X^{20b}, X^{21b} and X^{22b} are N; or three of X^{18b}, X^{19b}, X^{20b}, X^{21b} and X^{22b} are N; or four of X^{18b}, X^{19b}, X^{20b}, X^{21b} and X^{22b} are N. In certain embodiments, one of X^{18b}, X^{19b}, X^{20b}, X^{21b} and X^{22b} is N and the others are C; or two of X^{18b}, X^{19b}, X^{20b}, X^{21b} and X^{22b} are N and the others are C; or three of X^{18b}, X^{19b}, X^{20b}, X^{21b} and X^{22b} are N and the others are C; or four of X^{18b}, X^{19b}, X^{20b}, X^{21b} and X^{22b} are N and the others are C. For embodiments in which ring member atom X^{18b}, X^{19b}, X^{20b}, X^{21b} or X^{22b} is O or S, R groups shown in Formula E3 as bonded to the ring member atom may be absent. For example, where X^{19b} is O or S, R¹⁹ and R^{19A} may be absent; where X^{20b} is O or S, R²⁰ and R^{20A} may be absent; where X^{21b} is O or S, R²¹ and R^{21A} may be absent; or where X^{22b} is O or S, R²² and R^{22A} may be absent. For embodiments in which ring member atom X^{19b}, X^{20b}, X^{21b} or X^{22b} is S, the S may be in a reduced state (S) or an oxidized state (i.e., S(O) or SO₂). In certain embodiments, one of X^{19b}, X^{20b}, X^{21b} and X^{22b} is O or S; or two of X^{19b}, X^{20b}, X^{21b} and X^{22b} are O or S; or three of X^{19b}, X^{20b}, X^{21b} and X^{22b} are O or S. In certain embodiments, one of X^{19b}, X^{20b}, X^{21b} and X^{22b} is O or S and the others are C; or two of X^{19b}, X^{20b}, X^{21b} and X^{22b} are O or S and the others are C; or three of X^{19b}, X^{20b}, X^{21b} and X^{22b} are O or S and the others are C. In certain embodiments, zero, one or two of X^{18b}, X^{19b}, X^{20b}, X^{21b} and X^{22b} in Formula E3 is N and X^{18b}, X^{19b}, X^{20b}, X^{21b} and X^{22b} that are not N are C. In certain instances, Z^{5b} is an optionally substituted cycloalkyl and X^{18b}, X^{19b}, X^{20b}, X^{21b} and X^{22b} each is C.

In certain embodiments, a compound contains a R^{W} group according to Formula B2, Formula C2, Formula D2, Formula E2, Formula B3, Formula C3, Formula D3 or Formula E3, and R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R²⁰, R²¹, R^{21A}, R²² and R^{22A} each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; and
R^{14B}, R^{15B}, R^{16B}, R^{17B}, R^{18B}, R^{19B}, R^{20B}, R^{21B} and R^{22B} each independently is hydrogen, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl, or —C(O)OR^{u}, where R^{u} is defined for Formula A1.

In certain embodiments, a compound contains a R^{W} group according to Formula B2, Formula C2, Formula D2, Formula E2, Formula B3, Formula C3, Formula D3 or Formula E3, and R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{2A}, R²¹, R^{21A}, R²² and R^{22A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, hydroxy, halo, cyano, nitro, optionally substituted C5-C6 cycloalkyl, optionally substituted heterocycloalkyl containing 5 or 6 ring member atoms; where R^{g}, R^{h}, R^{j} and R^{k} each independently is hydrogen or optionally substituted C1-C6 alkyl. In certain instances, R^{g}, R^{h}, R^{j} and R^{k} each independently is hydrogen, optionally substituted C1-C4 alkyl, butyl, iso-butyl, tert-butyl, propyl, isopropyl, ethyl or methyl. In certain instances, R^{u} is an optionally substituted C1-C4 alkyl, butyl, iso-butyl, tert-butyl, propyl, isopropyl, ethyl or methyl.

In certain embodiments, a compound contains a R^{W} group according to Formula B2, Formula C2, Formula D2, Formula E2, Formula B3, Formula C3, Formula D3 or Formula E3, and R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{20A}, R²¹, R^{21A}, R²² and R^{22A} each independently is hydrogen, optionally substituted C1-C4 alkylamine, optionally substituted C1-C4 hydroxyalkylamine, HO—CH₂CH₂—N(H)—, halo, fluoro, chloro, optionally substituted C1-C4 alkyl, butyl, tert-butyl, iso-butyl, propyl, iso-propyl, ethyl, methyl, optionally substituted C1-C4 alkoxy, substituted C1-C4 alkoxy, unsubstituted benzyloxy, isopropyloxy, ethoxy, methoxy, nitro, —C(O)R^{z}, —C(O)OR^{u} or —C(O)OH, where R^{u} can be optionally substituted C1-C6 alkyl, optionally substituted C1-C4 alkyl, butyl, tert-butyl, iso-butyl, propyl, iso-propyl, ethyl or methyl. In certain instances, a compound contains a R^{W} group, the R^{W} group is an optionally substituted aryl or optionally substituted heteroaryl of Formula B2, and: R¹⁶ is an optionally substituted C1-C4 alkylamine, optionally substituted C1-C4 hydroxyalkylamine, or HO—CH₂CH₂—N(H)—; R¹⁵ or R¹⁶ is halo, fluoro or chloro; one, two or three of R¹⁴, R¹⁶ and R¹⁸ is an optionally substituted C1-C4 alkyl, ethyl, methyl, optionally substituted C1-C4 alkoxy, isopropyloxy, ethoxy or methoxy; R¹⁶ is hydroxy; R¹⁴ is —C(O)H; R¹⁴ is —C(O)R^{z}; R¹⁶ is an optionally substituted C1-C4 alkoxy, substituted C1-C4 alkoxy, or unsubstituted benzyloxy; R¹⁵ is nitro; and/or R¹⁷ is —C(O)OH.

In certain embodiments, a compound contains a R^{W} group according to Formula B2, Formula C2, Formula D2, Formula E2, Formula B3, Formula C3, Formula D3 or Formula E3, and R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R²⁰, R²¹, R^{21A}, R²² and R^{22A} each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, a compound contains a R^{W} group according to Formula B2, Formula C2, Formula D2, Formula E2, Formula B3, Formula C3, Formula D3 or Formula E3, and R^{14B}, R^{15B}, R^{16B}, R^{17B}, R^{18B}, R^{19B}, R^{20B}, R^{21B} and R^{22B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u}, where R^{u} is an optionally substituted C1-C6 alkyl, optionally substituted C1-C4 alkyl, butyl, tert-butyl, iso-butyl, propyl, iso-propyl, ethyl or methyl. In certain embodiments, a compound contains a R^{W} group according to Formula B2, Formula C2, Formula D2, Formula E2, Formula B3, Formula C3, Formula D3 or Formula E3, and R^{14B}, R^{15B}, R^{16B}, R^{17B}, R^{18B}, R^{19B}, R^{20B}, R^{21B} and R^{22B} each independently is hydrogen, optionally substituted C1-C6 alkyl, optionally substituted C1-C4 alkyl, butyl, tert-butyl, iso-butyl, propyl, iso-propyl, ethyl or methyl.

In certain embodiments, (i) one, two, three or four of R¹⁰, R¹¹, R¹² and R¹³ each is hydrogen, (ii) one, two or three of R¹⁵, R¹⁷ and R¹⁸ each is hydrogen, (iii) one or two of R¹⁴ and R¹⁶ each is hydrogen, and/or (iv) one or both of R²¹ and R²² each is hydrogen.

R¹, R², R³ and R⁴ Substituents and Other Positions

In certain embodiments, R¹, R², R³ and R⁴ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, hydroxy, halo, cyano or nitro; and R^{p}, R^{q}, R^{r} and R^{s} each independently is hydrogen or optionally substituted C1-C6 alkyl. In certain instances, R^{p}, R^{q}, R^{r} and R^{s} each independently is hydrogen, optionally substituted C1-C4 alkyl, butyl, iso-butyl, tert-butyl, propyl, isopropyl, ethyl or methyl. In certain embodiments, R^{u} is an optionally substituted C1-C4 alkyl, butyl, iso-butyl, tert-butyl, propyl, isopropyl, ethyl or methyl. In certain embodiments, R¹, R², R³ and R⁴ each independently is hydrogen, optionally substituted C1-C4 alkyl, optionally substituted C1-C4 alkoxy, ethyl, ethoxy, methyl, methoxy, Cl, F, CF₃ or CDs. In certain instances, R¹ is hydrogen. In certain instances, one of R², R³ and R⁴ is an optionally substituted C1-C4 alkyl, optionally substituted C1-C4 alkoxy, ethyl, ethoxy, methyl, methoxy, Cl, F, CF₃ or CDs, and the other two of R², R³ and R⁴ are hydrogen.

In certain embodiments, R² is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy; or R² is hydrogen, unsubstituted C1-C4 alkyl, unsubstituted C1-C4 deuteroalkyl, unsubstituted C1-C4 haloalkyl, unsubstituted C1-C4 alkylamino, or halo; or R² is unsubstituted C1-C4 alkoxy; or R² is hydrogen, unsubstituted C1-C4 alkyl, Cl, F, CF₃, CDs, unsubstituted C1-C4 alkoxy, methoxy, isopropyloxy or dimethylamino; or R² is unsubstituted C1-C4 alkyl, ethyl or methyl.

In certain instances, two, three or four of R¹, R², R³ and R⁴ are hydrogen. In certain instances, R² is methyl, ethyl, methoxy or ethoxy, and R¹, R³ and R⁴ each is hydrogen. In certain instances, R³ is methyl, ethyl, methoxy or ethoxy, and R¹, R² and R⁴ each is hydrogen. In certain instances, R⁴ is methyl, ethyl, methoxy or ethoxy, and R¹, R² and R³ each is hydrogen.

### Subgroups

In certain aspects, provided is a compound of the following Subgroup 1, Subgroup 2, Subgroup 3, Subgroup 4, Subgroup 5, Subgroup 6, Subgroup 7, Subgroup 8, Subgroup 9 or Subgroup 10.

### Subgroup 1: Certain Formula A1-3 Compounds

Provided in certain aspects is a compound of Formula A1-3:

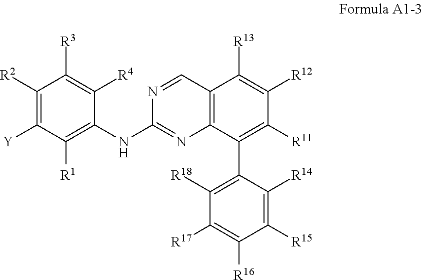

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{b})C(O)—R^{Y} or —N(R^{b})C(O)—R^{V}—R^{Y};
R^{Y} is an optionally substituted heterocycloalkyl containing six ring atoms;
R^{V} is a substituted C1-C4 alkylene or unsubstituted C1-C4 alkylene;
R^{b}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain embodiments, R¹, R², R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, (i) R¹⁴ and R¹⁶ each independently is hydrogen, C1-C4 alkyl, Cl, F, CF₃, CD₃, C1-C4 alkoxy, methoxy, isopropyloxy or dimethylamino; (ii) R¹⁴ is C1-C4 alkoxy; (iii) R¹⁴ is methoxy or isopropyloxy; (iv) R¹⁶ is chloro or fluoro; and/or (v) R¹⁶ is fluoro. In certain embodiments, R¹, R¹¹, R¹² and R¹³ each independently is hydrogen; R¹⁵, R¹⁷ and R¹⁸ each independently is hydrogen; R³ and R⁴ each independently is hydrogen; and/or R^{b} is hydrogen. In certain embodiments, R² is methyl, ethyl, methoxy or ethoxy, and R¹, R³ and R⁴ each is hydrogen. In certain embodiments, R^{V} is unsubstituted ethylene or unsubstituted methylene. In certain embodiments, R^{p}, R^{q}, R^{r} and R^{s} each independently is hydrogen or methyl. In certain embodiments, R^{u} is ethyl or methyl.

In certain embodiments, R^{Y} is an optionally substituted piperidinyl, optionally substituted piperazinyl or optionally substituted morpholinyl. In certain instances, R^{Y} is a substituted piperidinyl or substituted piperazinyl, which sometimes is substituted by an optionally substituted C1-C6 alkyl, ethyl or methyl at one, two or three ring atoms. In certain instances, R^{Y} is an unsubstituted morpholinyl. In certain embodiments, Y is —N(R^{b})C(O)—R^{V}—R^{Y}. In certain embodiments, R^{Y} is of Formula D1 and X¹² is N; X¹³ is C(R⁵)R^{5A}; X¹⁴ is C(R⁶)R^{6A}; X¹⁵ is N—R^{7B} X¹⁶ is C(R⁸)R^{8A}, and X¹⁷ is C(R⁹)R^{9A}. In certain embodiments, Y is —N(R^{b})C(O)—R^{Y}. In certain instances, R^{Y} is of Formula D1 and X¹² is C—R^{aA}; X¹³ is C(R⁵)R^{5A}; X¹⁴ is C(R⁶)R^{6A}; X¹⁵ is N—R^{7B}; X¹⁶ is C(R⁸)R^{8A}; and X¹⁷ is C(R⁹)R^{9A}. In certain embodiments, R^{aA}, R⁵, R^{5A}, R⁶, R^{6A}, R⁸, R^{8A}, R⁹ and R^{9A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{k}R^{j}, —C(O)R^{z}, —C(O)OH, —C(O)OR^{u}, —B(OH)₂, hydroxy, halo, nitro or cyano; where R^{g}, R^{h}, R^{k} and R^{j} each independently is hydrogen or optionally substituted C1-C6 alkyl; and R^{u} is an optionally substituted C1-C6 alkyl. In certain embodiments, R^{7B} is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u}. In certain embodiments, (i) R^{aA}, R⁵, R^{5A}, R⁶, R^{6A}, R^{7B}, R⁸, R^{8A}, R⁹ and R^{9A} each independently is hydrogen or optionally substituted C1-C4 alkyl; (ii) R^{u} is an optionally substituted C1-C4 alkyl; (iii) R^{g}, R^{h}, R^{k} and R^{j} each independently is hydrogen or optionally substituted C1-C4 alkyl; (iv) the optionally substituted C1-C4 alkyl of (i), (ii) or (iii) is an unsubstituted C1-C4 alkyl; and/or (v) the unsubstituted C1-C4 alkyl of (iv) is butyl, tert-butyl, iso-butyl, propyl, isopropyl, ethyl or methyl. In certain instances, R^{aA}, R⁵, R^{5A}, R⁶, R^{6A}, R^{7B}, R⁸, R^{8A}, R⁹ and R^{9A} each independently is hydrogen or optionally substituted C1-C4 alkyl. In certain instances, R^{aA}, R⁵, R^{5A}, R⁶, R^{6A}, R⁸, R^{8A}, R⁹ and R^{9A} each is hydrogen and R^{7B} is methyl.

In certain embodiments, Y is:

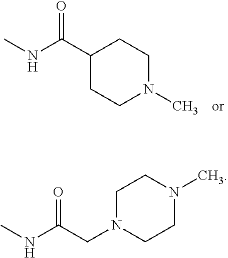

A representative Subgroup 1 compound is:
N-(5-((8-(4-fluoro-2-isopropoxyphenyl)quinazolin-2-yl)amino)-2-methylphenyl)-1-methylpiperidine-4-carboxamide; or
(S)—N-(5-((8-(4-fluoro-2-isopropoxyphenyl)quinazolin-2-yl)amino)-2-methylphenyl)-1-methylpyrrolidine-2-carboxamide; or
N-(5-((8-(4-fluoro-2-isopropoxyphenyl)quinazolin-2-yl)amino)-2-methylphenyl)-1-methylazetidine-3-carboxamide; or
N-(5-((8-(4-fluoro-2-isopropoxyphenyl)quinazolin-2-yl)amino)-2-methylphenyl)tetrahydro-2H-pyran-4-carboxamide; or
(R)—N-(5-((8-(4-fluoro-2-isopropoxyphenyl)quinazolin-2-yl)amino)-2-methylphenyl)-1-methylpyrrolidine-2-carboxamide; or
N-(5-((8-(4-fluoro-2-isopropoxyphenyl)quinazolin-2-yl)amino)-2-methylphenyl)-2-(4-methylpiperazin-1-yl)acetamide; or
N-(5-((8-(4-fluoro-2-isopropoxyphenyl)quinazolin-2-yl)amino)-2-methylphenyl)-4-methylpiperazine-1-carboxamide; or
   or a pharmaceutically acceptable salt thereof.

### Subgroup 2: Certain Formula A1-3 Compounds

Provided in certain aspects is a compound of Formula A1-3:

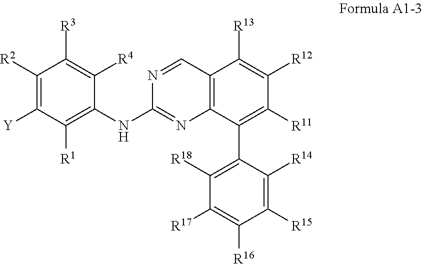

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{a})R^{b};
R^{a}, R^{b}, R^{p}, R^{q}, R^{r} and R^{s} each independently is hydrogen or optionally substituted C1-C6 alkyl;
R^{z} is hydrogen or R^{u}; and
R^{u} is or optionally substituted C1-C6 alkyl.

In certain embodiments, R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy. In certain embodiments, R¹, R², R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino. In certain embodiments, (i) R¹⁴ and R¹⁶ each independently is hydrogen, C1-C4 alkyl, Cl, F, CF₃, CD₃, C1-C4 alkoxy, methoxy, isopropyloxy or dimethylamino; (ii) R¹⁴ is C1-C4 alkoxy; (iii) R¹⁴ is methoxy or isopropyloxy; (iv) R¹⁶ is chloro or fluoro; and/or (v) R¹⁶ is fluoro.

In certain embodiments, R² is an optionally substituted C1-C4 alkoxy, optionally substituted C1-C4 alkyl or methyl. In certain embodiments, R¹, R¹¹, R¹² and R¹³ each is hydrogen. In certain instances, R² is methyl, ethyl, methoxy or ethoxy, and R¹, R³ and R⁴ each is hydrogen. In certain embodiments, R¹⁵, R¹⁷ and R¹⁸ each is hydrogen. In certain embodiments, R³ and R⁴ each is hydrogen. In certain embodiments, R² is hydrogen. In certain embodiments, R^{p}, R^{q}, R^{r} and R^{s} each independently is hydrogen, ethyl or methyl. In certain embodiments, R^{a} and R^{b} each independently is hydrogen or C1-C4 alkyl, ethyl or methyl. In certain embodiments, R^{a} and R^{b} each is hydrogen. In certain embodiments, R^{a} and R^{b} are not joined and do not form an unsubstituted heterocycloalkyl or substituted heterocycloalkyl.

A representative Subgroup 2 compound is:

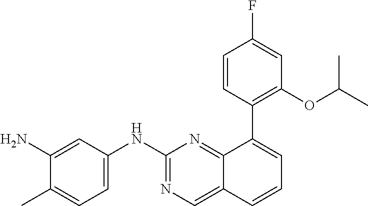 [SMILES: Cc1ccc(Nc2ncc3cccc(-c4ccc(F)cc4OC(C)C)c3n2)cc1N]

or a pharmaceutically acceptable salt thereof.

### Subgroup 3: Certain Formula A1-3 Compounds

Provided in certain aspects is a compound of Formula A1-3:

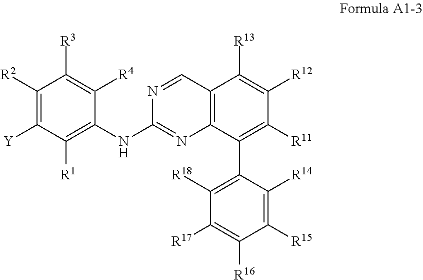

or a pharmaceutically acceptable salt, amide or ester thereof, where:
R¹, R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
Y is —N(R^{b})C(O)—R^{Y};
R^{Y} is an optionally substituted alkenyl;
R² is hydrogen, optionally substituted C1-C6 alkyl, unsubstituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —N(H)R^{r}, —NH₂, —C(O)R^{z}, —C(O)OH, —C(O)OR^{u}, —B(OH)₂, hydroxy, halo, nitro or cyano;
R^{b}, R^{g}, R^{h}, R^{j}, R^{k}, R^{p} and R^{q} each independently is hydrogen or optionally substituted C1-C6 alkyl;
R^{r} and R^{s} each independently is hydrogen or unsubstituted C1-C6 alkyl;
R^{z} is hydrogen or R^{u}; and
R^{u} is an optionally substituted C1-C6 alkyl.

In certain embodiments, R^{Y} is an optionally substituted C2-C4 alkenyl. In certain embodiments, R^{Y} is —CH═CH₂. In certain embodiments, R¹, R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy. In certain embodiments, R¹, R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino. In certain embodiments, (i) R¹⁴ and R¹⁶ each independently is hydrogen, C1-C4 alkyl, C, F, CF₃, CD₃, C1-C4 alkoxy, methoxy, isopropyloxy or dimethylamino; (ii) R¹⁴ is C1-C4 alkoxy; (iii) R¹⁴ is methoxy or isopropyloxy; (iv) R¹⁶ is chloro or fluoro; and/or (v) R¹⁶ is fluoro.

In certain embodiments, R² is hydrogen, C1-C4 alkyl, C1-C4 deuteroalkyl, C1-C4 haloalkyl, C1-C4 alkylamino or halo. In certain embodiments, R² is hydrogen, unsubstituted C1-C4 alkyl, unsubstituted C1-C4 deuteroalkyl, unsubstituted C1-C4 haloalkyl, unsubstituted C1-C4 alkylamino or halo. In certain embodiments, R² is hydrogen, unsubstituted C1-C4 alkyl, Cl, F, CF₃, CD₃, or dimethylamino. In certain embodiments, R² is unsubstituted C1-C4 alkoxy, ethoxy or methoxy. In certain embodiments, R² is unsubstituted C1-C4 alkyl, ethyl or methyl. In certain instances, R² is methyl or ethyl, and R¹, R³ and R⁴ each is hydrogen. In certain instances, R² is methoxy or ethoxy, and R¹, R³ and R⁴ each is hydrogen. In certain embodiments, R¹, R¹¹, R¹² and R¹³ each independently is hydrogen. In certain embodiments, R¹⁵, R¹¹ and R¹⁸ each independently is hydrogen. In certain embodiments, R³ and R⁴ each independently is hydrogen. In certain embodiments, R² is hydrogen. In certain embodiments, R^{b} is hydrogen. In certain embodiments, R^{b}, R^{k}, R^{m}, R^{p} and R^{q} each independently is hydrogen or methyl. In certain embodiments, R^{b} and R^{Y} are not joined and do not form an unsubstituted heterocycloalkyl or substituted heterocycloalkyl.

A representative Subgroup 3 compound is

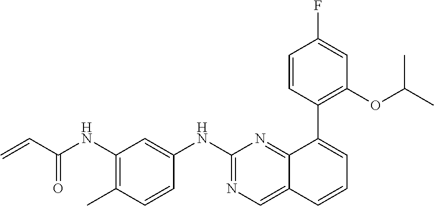 [SMILES: C=CC(=O)Nc1cc(Nc2ncc3cccc(-c4ccc(F)cc4OC(C)C)c3n2)ccc1C]

or a pharmaceutically acceptable salt thereof.

### Subgroup 4: Certain Formula A1-8 Compounds

Provided in certain aspects is a compound of Formula A1-8:

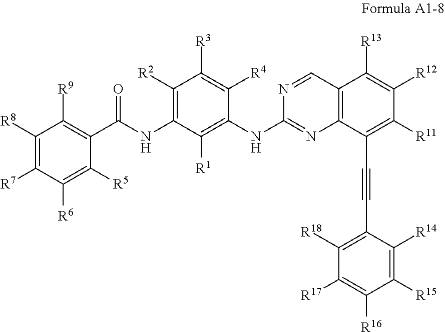

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R⁹C(O)N(R^{h})—, —C(O)N(R⁹R^{h}), —NR^{j}R^{k}, —C(O)R^{z}, —C(O)OH, —C(O)OR^{u}, —B(OH)₂, hydroxy, halo, nitro or cyano, R⁵, R⁶, R⁷, R⁸ and R⁹ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{b}, R^{g}, R^{h}, R^{j}, R^{k}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R⁵, R⁶, R⁷, R⁸, R⁹, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain embodiments, R¹, R², R³, R⁴, R⁵, R⁶, R⁷, R⁸, R⁹, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino. In certain embodiments, R¹, R¹¹, R¹² and R¹³ each is hydrogen. In certain embodiments, R^{b} is hydrogen; R¹⁵, R¹⁷ and R¹⁸ each is hydrogen. In certain embodiments, R³ and R⁴ each is hydrogen. In certain embodiments, R² is hydrogen. In certain embodiments, R⁵, R⁶, R⁸ and R⁹ each is hydrogen. In certain embodiments, R² is an optionally substituted C1-C4 alkoxy, optionally substituted C1-C4 alkyl or methyl. In certain instances, R² is methyl, ethyl, methoxy or ethoxy, and R¹, R³ and R⁴ each is hydrogen. In certain embodiments, R⁷ is —C(O)OH or —C(O)OR^{u}. In certain embodiments, R⁷ is —C(O)OH. In certain embodiments, R⁷ is R^{p}C(O)N(R^{q})— or —C(O)N(R^{p}R^{q}) and R^{p} and R^{q} each independently is hydrogen, optionally substituted C1-C4 alkyl, ethyl or methyl. In certain embodiments, R^{u} is an optionally substituted C1-C4 alkyl, ethyl or methyl. In certain embodiments, R^{g}, R^{h}, R^{j}, R^{k}, R^{p}, R^{q}, R^{r} and R^{s} each independently is hydrogen or methyl. In certain embodiments: (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is hydrogen, chloro or fluoro; and/or (v) R¹⁶ is hydrogen.

A representative Subgroup 4 compound is:

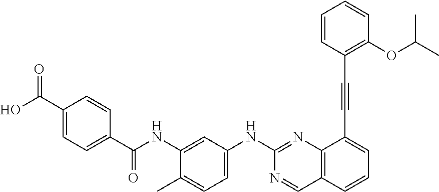 [SMILES: Cc1ccc(Nc2ncc3cccc(C#Cc4ccccc4OC(C)C)c3n2)cc1NC(=O)c1ccc(C(=O)O)cc1]

or a pharmaceutically acceptable amide, ester or salt thereof.

### Subgroup 5: Certain Formula A1-5 Compounds

Provided in certain aspects is a compound of Formula A1-5:

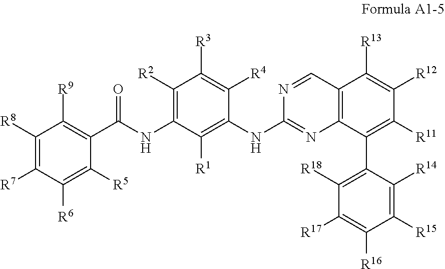

or a pharmaceutically acceptable salt, amide or ester thereof, where:
R⁷ is —C(O)N(R^{c}R^{d}) or is a Formula F group;
R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Z¹ is an optionally substituted heterocycloalkyl; X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{c}, R^{d}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain embodiments, R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, R¹, R¹¹, R¹² and R¹³ each is hydrogen. In certain embodiments, R¹⁵, R¹⁷ and R¹⁸ each is hydrogen. In certain embodiments, R³ and R⁴ each is hydrogen. In certain embodiments, R² is hydrogen. In certain embodiments, R⁵, R⁶, R⁸ and R⁹ each is hydrogen. In certain embodiments, R² is an optionally substituted C1-C4 alkoxy, optionally substituted C1-C4 alkyl or methyl. In certain instances, R² is methyl, ethyl, methoxy or ethoxy, and R¹, R³ and R⁴ each is hydrogen.

In certain embodiments, R⁷ is —C(O)N(R^{c}R^{d}) and R^{c} and R^{d} each independently is hydrogen or optionally substituted C1-C4 alkyl; or R^{c} and R^{d} each independently is hydrogen or unsubstituted C1-C4 alkyl; or R^{c} and R^{d} each is unsubstituted C1-C4 alkyl. In certain embodiments, R⁷ is —C(O)N(CH₃)CH₃, —C(O)N(H)CH₃ or —C(O)NH₂.

In certain embodiments, R⁷ is a Formula F group. In certain embodiments, X^{a} is C(R⁴³)R^{43A}; X^{b} is C(R⁴⁴)R^{44A}; X^{c} is N—R^{45B}; X^{d} is C(R⁴⁶)R^{46A}; and X^{e} is C(R⁴⁷)R^{47A}. In certain embodiments, R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, ethyl or methyl. In certain embodiments, R⁴³, R⁴⁴, R⁴⁵, R⁴⁶, R⁴⁷, R^{43A}, R^{44A}, R^{46A} and R^{47A} each is hydrogen and R^{45A} and R^{45B} each is hydrogen, optionally substituted C1-C6 alkyl, ethyl or methyl. In certain embodiments, R⁴³, R⁴⁴, R⁴⁵, R⁴⁶, R⁴⁷, R^{43A}, R^{44A}, R^{46A} and R^{47A} each is hydrogen and R^{45A} and R^{45B} is methyl. In certain embodiments, R⁷ is:

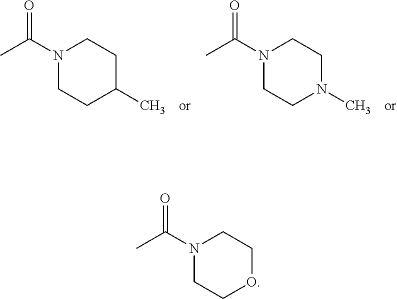

In certain embodiments, R^{u} is an optionally substituted C1-C4 alkyl, butyl, tert-butyl, iso-butyl, propyl, isopropyl, ethyl or methyl. In certain embodiments, R^{p}, R^{q}, R^{r} and R^{s} each independently is hydrogen or methyl. In certain embodiments: (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is hydrogen, chloro or fluoro; and/or (v) R¹⁶ is fluoro.

A representative Subgroup 5 compound is:
N-(5-((8-(4-fluoro-2-isopropoxyphenyl)quinazolin-2-yl)amino)-2-methylphenyl)terephthalamide; or
N1-(5-((8-(4-fluoro-2-isopropoxyphenyl)quinazolin-2-yl)amino)-2-methylphenyl)-N4-methylterephthalamide; or
N1-(5-((8-(4-fluoro-2-isopropoxyphenyl)quinazolin-2-yl)amino)-2-methylphenyl)-N4,N4-dimethylterephthalamide; or
N-(5-((8-(4-fluoro-2-isopropoxyphenyl)quinazolin-2-yl)amino)-2-methylphenyl)-4-(4-methylpiperazine-1-carbonyl)benzamide; or
N-(5-((8-(4-fluoro-2-isopropoxyphenyl)quinazolin-2-yl)amino)-2-methylphenyl)-4-(morpholine-4-carbonyl)benzamide; or
   or a pharmaceutically acceptable amide, ester or salt thereof.

### Subgroup 6: Certain Formula A1-3 Compounds

In certain aspects, provided is a compound of Formula A1-3:

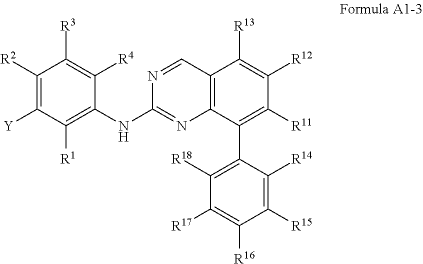

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{b})C(O)—R^{Y};
R^{Y} is an unsubstituted C1-C6 alkyl or unsubstituted C1-C6 deuteroalkyl;
R^{b}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain instances, R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro. In certain instances, one, two, three or four of R¹, R¹¹, R¹² and R¹³ each is hydrogen. In certain embodiments, one, two or three of R¹⁵, R¹⁷ and R¹⁸ each is hydrogen. In certain embodiments, one or two of R³ and R⁴ each is hydrogen. In certain embodiments, R^{b} is hydrogen. In certain instances, R² is methyl, ethyl, methoxy or ethoxy, and R¹, R³ and R⁴ each is hydrogen. In certain embodiments, (i) R^{V} is unsubstituted ethylene or unsubstituted methylene; (ii) R^{p}, R^{q}, R^{r} and R^{s} each independently is hydrogen or methyl; and/or (iii) R^{u} is ethyl or methyl. In certain instances, R^{Y} is methyl or ethyl. In certain embodiments, R^{Y} is methyl.

In certain embodiments, provided is a compound of formula:
N-(5-((8-(4-fluoro-2-isopropoxyphenyl)quinazolin-2-yl)amino)-2-methylphenyl)acetamide; or
N-(5-((8-(4-fluoro-2-isopropoxyphenyl)quinazolin-2-yl)amino)-2-methoxyphenyl)acetamide; or
   or a pharmaceutically acceptable salt thereof.

### Subgroup 7: Certain Formula A1-6 Compounds

Provided in certain aspects compound of Formula A1-6:

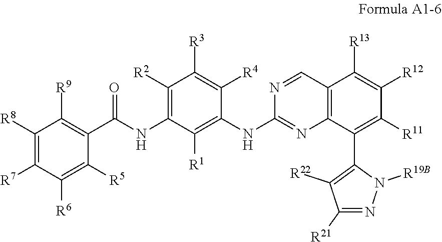

or a pharmaceutically acceptable salt, amide or ester thereof, where:
R¹, R², R³, R⁴, R¹¹, R¹², R¹³, R²¹ and R²² each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
R⁵, R⁶, R⁸ and R⁹ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R⁷ is R^{c}C(O)N(R^{d})— or —C(O)N(R^{c}R^{d});
R^{19B} is hydrogen, optionally substituted C1-C6 alkyl, optionally substituted C1-C4 alkyl, butyl, tert-butyl, iso-butyl, propyl, iso-propyl, ethyl or methyl;
R^{c}, R^{d}, R^{g}, R^{h}, R^{j}, R^{k}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R⁵, R⁶, R⁸, R⁹, R¹¹, R¹², R¹³, R²¹ and R²² each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain instances, R¹, R², R³, R⁴, R⁵, R⁶, R⁸, R⁹, R¹¹, R¹², R¹³, R²¹ and R²² each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, one, two, three or four of R¹, R¹¹, R¹² and R¹³ each is hydrogen. In certain embodiments, one, two of three of R^{19B}, R²¹ and R²² each is hydrogen. In certain embodiments, one or two of R³ and R⁴ each is hydrogen. In certain embodiments, one, two, three or four of R⁵, R⁶, R⁸ and R⁹ each is hydrogen. In certain embodiments, R² is hydrogen. In certain instances, R² is methyl, ethyl, methoxy or ethoxy, and R¹, R³ and R⁴ each is hydrogen.

In certain embodiments, R^{c} and R^{d} each independently is hydrogen or unsubstituted C1-C4 alkyl; or R^{c} and R^{d} each independently is hydrogen or methyl; or R^{c} is hydrogen and R^{d} is unsubstituted C1-C4 alkyl; or R^{c} is hydrogen and R^{d} is methyl; or R^{c} and R^{d} each is unsubstituted C1-C4 alkyl; or R^{c} and R^{d} each is methyl. In certain instances, R⁷ is —C(O)N(CH₃)CH₃, —C(O)N(H)CH₃ or —C(O)NH₂. In certain embodiments, R⁷ is —C(O)N(H)CH₃.

In certain embodiments, R^{g}, R^{h}, R^{j}, R^{k}, R^{r} and R^{s} each independently is hydrogen or methyl. In certain instances: (i) R²¹ and R²² each independently is hydrogen, unsubstituted C1-C4 alkyl, Cl, F, CF₃, CD₃, unsubstituted C1-C4 alkoxy, methoxy, isopropyloxy or dimethylamino; (ii) one or two of R²¹ and R²² each is hydrogen; (iii) R^{19B} is an optionally substituted C1-C6 alkyl; (iv) R^{19B} is unsubstituted C1-C6 alkyl; and/or (v) R^{19B} is butyl, iso-butyl, tert-butyl, propyl, iso-propyl, ethyl or methyl.

In certain embodiments, provided is a compound of formula:
N1-(5-((5-(1-isobutyl-1H-pyrazol-5-yl)quinazolin-2-yl)amino)-2-methylphenyl)-N4-methylterephthalamide; or
N1-(5-((8-(1-isobutyl-1H-pyrazol-5-yl)quinazolin-2-yl)amino)-2-methylphenyl)-N4,N4-dimethylterephthalamide; or
   or a pharmaceutically acceptable amide, ester or salt thereof.

### Subgroup 8: Certain Formula A1-3 Compounds

Provided in certain aspects is a compound of Formula A1-3:

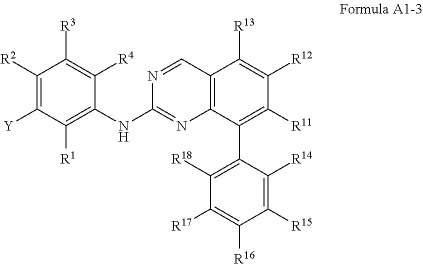

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is of Formula F:

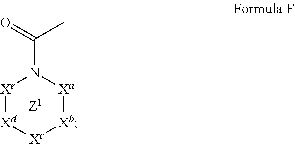

Z¹ is an optionally substituted heterocycloalkyl; X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{p}, R^{q}, R^{r} and R^{s} each independently is hydrogen or optionally substituted C1-C6 alkyl;
R^{z} is hydrogen or R^{u}; and
R^{u} is or optionally substituted C1-C6 alkyl.

In certain embodiments, R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy. In certain instances, R¹, R², R³, R⁴, R¹¹, R¹², R¹³, R¹⁴, R¹⁵, R¹⁶, R¹⁷ and R¹⁸ each independently is hydrogen, unsubstituted C1—C4 alkyl, Cl, F, CF₃, CD₃, unsubstituted C1-C4 alkoxy, methoxy, isopropyloxy or dimethylamino.

In certain instances, (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro. In certain embodiments, R² is an optionally substituted C1-C4 alkoxy, methoxy, optionally substituted C1-C4 alkyl or methyl. In certain instances, R² is methyl, ethyl, methoxy or ethoxy, and R¹, R³ and R⁴ each is hydrogen. In certain instances, one, two three or four of R¹, R¹¹, R¹² and R¹³ each is hydrogen. In certain embodiments, one, two or three of R¹⁵, R¹⁷ and R¹⁸ each is hydrogen. In certain embodiments, one or two of R³ and R⁴ each is hydrogen. In certain embodiments, R² is hydrogen. In certain embodiments, R^{p}, R^{q}, R^{r} and R^{s} each independently is hydrogen, ethyl or methyl.

In certain instances, Y is of Formula F and X^{a} is C(R⁴³)R^{43A}; X^{b} is C(R⁴⁴)R^{44A}; X^{c} is N—R^{45B}; X^{d} is C(R⁴⁶)R^{46A}; and X^{e} is C(R⁴⁷)R^{47A}. In certain embodiments, Y is of Formula F and R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, ethyl or methyl. In certain embodiments, Y is of Formula F and R⁴³, R⁴⁴, R⁴⁵, R⁴⁶, R⁴⁷, R^{43A}, R^{44A}, R^{46A} and R^{47A} each is hydrogen and R^{45A} and R^{45B} each is hydrogen, optionally substituted C1-C6 alkyl, ethyl or methyl. In certain embodiments, Y is Formula F and R⁴³, R⁴⁴, R⁴⁵, R⁴⁶, R⁴⁷, R^{43A}, R^{44A}, R^{46A} and R^{47A} each is hydrogen and R^{45A} and R^{45B} is methyl. In certain embodiments, Y is:

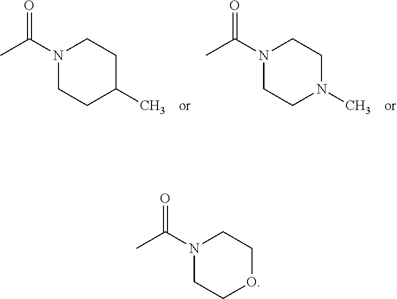

In certain instances, provided is a compound of formula:

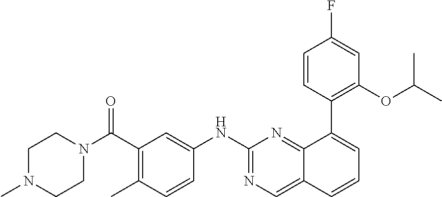 [SMILES: Cc1ccc(Nc2ncc3cccc(-c4ccc(F)cc4OC(C)C)c3n2)cc1C(=O)N1CCN(C)CC1]

or a pharmaceutically acceptable salt thereof.

### Subgroup 9: Certain Formula A1-2 Compounds

Provided in certain aspects is a compound of Formula A1-2:

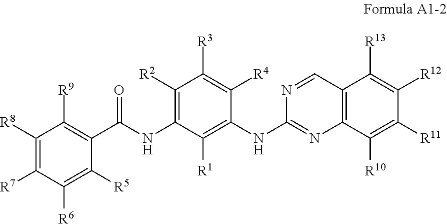

or a pharmaceutically acceptable salt, amide or ester thereof, where:
R¹⁰ is of Formula B2:

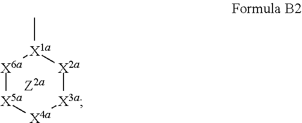

Z^{2a} is heteroaryl; and (i) X^{1a} is C, X^{2a} is C—R¹⁴, X^{3a} is N, X^{4a} is C—R¹⁶, X^{5a} is N, and X^{6a} is C—R¹⁸; or (ii) X^{1a} is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused optionally substituted heteroaryl containing five ring atoms;
R¹, R², R³, R⁴, R⁵, R⁶, R⁷, R⁸, R⁹, R¹¹, R¹², R¹³, R¹⁴, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl,
R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R⁵, R⁶, R⁸, R⁹, R¹¹, R¹², R¹³, R¹⁴, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain instances, R¹, R², R³, R⁴, R⁵, R⁶, R⁸, R⁹, R¹¹, R¹², R¹³, R¹⁴, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

In certain embodiments, one, two, three or four of R¹, R¹¹, R¹² and R¹³ each is hydrogen. In certain embodiments, one or two of R¹⁷ and R¹⁸ each is hydrogen. In certain embodiments, one or two of R³ and R⁴ each is hydrogen. In certain embodiments, R² is hydrogen. In certain embodiments, one, two, three or four of R⁵, R⁶, R⁸ and R⁹ each is hydrogen. In certain embodiments, R² is methyl, ethyl, methoxy or ethoxy, and R¹, R³ and R⁴ each is hydrogen.

In certain instances, R⁷ is —C(O)OH or —C(O)OR^{u}. In certain instances, R⁷ is —C(O)OH. In certain embodiments, R⁷ is —C(O)N(R^{c}R^{d}) and R^{c} and R^{d} each independently is hydrogen or optionally substituted C1-C4 alkyl. In certain instances, R^{c} and R^{d} each independently is hydrogen or unsubstituted C1-C4 alkyl; or R^{c} and R^{d} each independently is hydrogen or methyl; or R^{c} and R^{d} each is unsubstituted C1-C4 alkyl; or R^{c} and R^{d} each is methyl. In certain embodiments, R⁷ is —C(O)N(CH₃)CH₃, —C(O)N(H)CH₃ or —C(O)NH₂.

In certain instances, R^{u} is an optionally substituted C1-C4 alkyl, butyl, tert-butyl, iso-butyl, propyl, isopropyl, ethyl or methyl. In certain embodiments, R^{p}, R^{q}, R^{r} and R^{s} each independently is hydrogen, ethyl or methyl.

In certain instances, X^{1a} is C, X^{2a} is C—R¹⁴, X^{3a} is N, X^{4a} is C—R¹⁶, X^{5a} is N, and X^{6a} is C—R¹⁸. In certain embodiments: (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ and R¹⁶ each independently is hydrogen or unsubstituted C1-C4 alkoxy; (iii) R¹⁴ and R¹⁶ each independently is hydrogen or methoxy; (iv) R¹⁴ and R¹⁶ each is methoxy; and/or (v) R¹⁸ is hydrogen. In certain embodiments, R¹⁰ is:

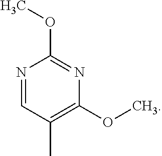

In certain instances, X^{1a} is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused optionally substituted pyrrolyl. In certain embodiments, Xia is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused unsubstituted pyrrolyl. In certain instances, one, two or three of R¹⁴, R¹⁷ or R¹⁸ each is hydrogen. In certain embodiments, R¹⁰ is:

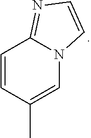

In certain embodiments, provided is a compound of formula:

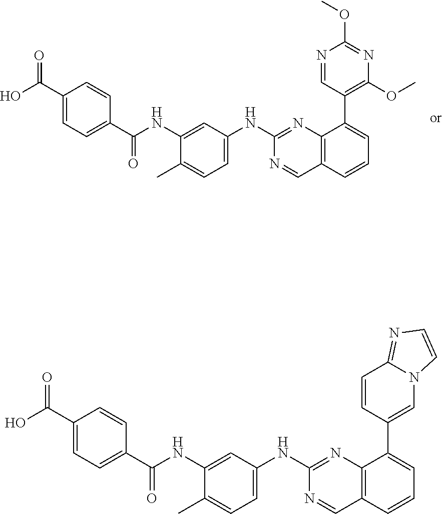 [SMILES: COc1ncc(-c2cccc3cnc(Nc4ccc(C)c(NC(=O)c5ccc(C(=O)O)cc5)c4)nc23)c(OC)n1 ; Cc1ccc(Nc2ncc3cccc(-c4ccc5nccn5c4)c3n2)cc1NC(=O)c1ccc(C(=O)O)cc1]

or a pharmaceutically acceptable amide, ester or salt thereof.

### Subgroup 10: Certain Formula A1-2 Compounds

Provided in certain aspects is a compound of Formula A1-2:

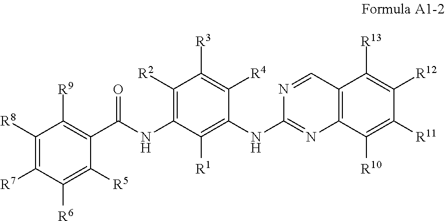

or a pharmaceutically acceptable salt, amide or ester thereof, where:
R¹¹ is of Formula C2:

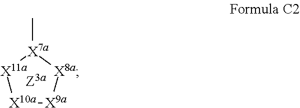

Z^{3a} is heteroaryl; and X^{7a} is C; X^{8a} is N or N—R^{19B}; X^{9a} is N or N—R^{20B}; X^{10a} is C—R²¹; and X^{11a} is C—R²²;
R¹, R², R³, R⁴, R⁵, R⁶, R⁷, R⁸, R⁹, R¹⁰, R¹², R¹³, R²¹ and R²² each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{19B} and R^{20B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH or —C(O)OR^{u};
R^{P}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

In certain embodiments, R¹, R², R³, R⁴, R⁵, R⁶, R⁸, R⁹, R¹⁰, R¹², R¹³, R²¹ and R²² each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy. In certain embodiments, R¹, R², R³, R⁴, R⁵, R⁶, R⁸, R⁹, R¹⁰, R¹², R¹³, R²¹ and R²² each independently is hydrogen, unsubstituted C1-C4 alkyl, Cl, F, CF₃, CD₃, unsubstituted C1-C4 alkoxy, methoxy, isopropyloxy or dimethylamino;

In certain embodiments, one, two, three or four of R¹, R¹¹, R¹² and R¹³ each is hydrogen. In certain embodiments, one, two, three or four of R^{19B}, R^{20B}, R²¹ and R²² each is hydrogen. In certain embodiments, one or two of R³ and R⁴ each is hydrogen. In certain embodiments, R² is hydrogen. In certain embodiments, one, two, three or four of R⁵, R⁶, R⁸ and R⁹ each is hydrogen. In certain embodiments, R² is methyl, ethyl, methoxy or ethoxy, and R¹, R³ and R⁴ each is hydrogen.

In certain embodiments, R⁷ is —C(O)OH or —C(O)OR^{u}. In certain embodiments, R⁷ is —C(O)OH. In certain embodiments, R⁷ is —C(O)N(R^{c}R^{d}) and R^{c} and R^{d} each independently is hydrogen or optionally substituted C1-C4 alkyl. In certain embodiments, R^{c} and R^{d} each independently is hydrogen or unsubstituted C1-C4 alkyl; or R^{c} and R^{d} each independently is hydrogen or methyl; or R^{c} and R^{d} each is unsubstituted C1-C4 alkyl; or R^{c} and R^{d} each is methyl. In certain embodiments, R⁷ is —C(O)N(CH₃)CH₃, —C(O)N(H)CH₃ or —C(O)NH₂.

In certain embodiments, R^{u} is an optionally substituted C1-C4 alkyl, butyl, tert-butyl, iso-butyl, propyl, isopropyl, ethyl or methyl. In certain embodiments, R^{p}, R^{q}, R^{r} and R^{s} each independently is hydrogen, ethyl or methyl.

In certain embodiments, R^{19B} and R^{20B} each independently is hydrogen, optionally substituted C1-C4 alkyl, optionally substituted C1-C4 deuteroalkyl, optionally substituted C1-C4 haloalkyl or optionally substituted C1-C4 alkylamino. In certain embodiments, (i) X^{8a} is N and X^{9a} is N—R^{20B}; (ii) R^{19B} and R^{20B} each independently is hydrogen, unsubstituted C1-C4 alkyl, ethyl or methyl; (iii) R²¹ and R²² each independently is hydrogen, unsubstituted C1-C4 alkyl, ethyl or methyl; (iv) R^{19B} and R^{20B} each is hydrogen; and/or (v) R²¹ and R²² each is hydrogen. In certain embodiments, R¹¹ is

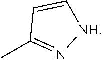

In certain embodiments, provided is a compound of formula:

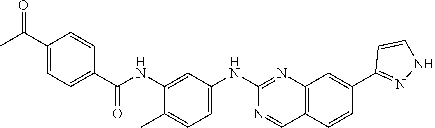 [SMILES: CC(=O)c1ccc(C(=O)Nc2cc(Nc3ncc4ccc(-c5cc[nH]n5)cc4n3)ccc2C)cc1]

or a pharmaceutically acceptable amide, ester or salt thereof.

A composition containing a compound of Subgroup 1, Subgroup 2, Subgroup 3, Subgroup 4, Subgroup 5, Subgroup 6, Subgroup 7, Subgroup 8, Subgroup 9 or Subgroup 10 sometimes is formulated for oral administration, topical administration (a cream, for example) or administration by injection or infusion.

In certain embodiments, a compound of Formula A1, Formula A1-1, Formula A1-2, Formula A1-3, Formula A1-4, Formula A1-5, Formula A1-6, Formula A1-7, Formula A1-8, Formula A2, Formula A2-1, Formula A2-2 or Formula A2-3 is provided with one or more of the following applicable provisos.

In certain embodiments, a compound is provided with the proviso that R⁷ is not —C(O)OH.

In certain embodiments, a compound is provided with the proviso that R² and R³, or optionally R³ and R⁴, are not joined as an imidazolyl group. In certain embodiments, a compound is provided with the proviso that R² and R³, or optionally R³ and R⁴, are not joined as: (i) an imidazolyl moiety fused to the phenyl group on which R², R³ and R⁴ are substituents; (ii) an indolyl group; (iii) a five-membered ring; (iv) a five-membered ring fused to the phenyl group on which R², R³ and R⁴ are substituents; (v) unsubstituted heteroaryl containing 5 ring atoms; and/or (vi) substituted heteroaryl containing 5 ring atoms.

In certain embodiments, a compound is provided with the proviso that R¹⁰ and/or optionally one, two or three of R¹¹, R¹² or R¹³, is not: methyl; substituted alkyl or unsubstituted alkyl; methoxy; substituted alkoxy or unsubstituted alkoxy; and/or unsubstituted phenyl. In certain embodiments, a compound is provided with the proviso that R¹¹ and R¹² each is not methoxy. In certain embodiments, a compound is provided, where R¹⁰, R¹¹, R¹² or R¹³ is —W—R^{W}, with the proviso that R^{W} is not a phenyl substituted with —C≡CH₃ and W is not amino.

In certain embodiments, a compound is provided with the proviso that R² and R⁹ do not form a bond (i.e., R² and R⁹ are not a bond). In certain embodiments, a compound is provided with the proviso that (i) the amino group joined by a covalent bond to the carbon ring atom in the quinazolinyl group, which carbon ring atom is positioned between the two nitrogen ring atoms of the quinazolinyl group, and (ii) R¹, do not participate in a five-membered ring, and/or do not join in an indolyl group.

In certain embodiments, a compound is provided where Y is —NR^{a}R^{b}, —N(R^{b})—CH₂—R^{Y} or —CH₂—N(R^{b})R^{Y}, with the proviso that: (i) R¹⁶ is not methoxy; (ii) R¹⁵ or R¹⁶ each independently is not

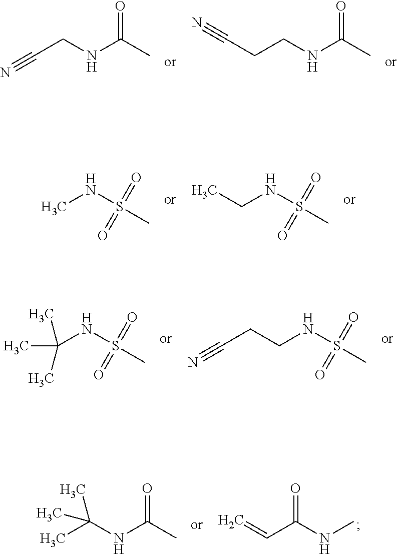

(iii) R¹⁵ or R¹⁶ each independently is not

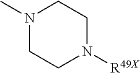

where R^{49X} is methyl, ethyl, methoxy, —C(O)CH₃, —CH₂CH₂OH, —CH₂CH₂OCH₃, —CH₂CH(OH)CH₃, —CH₂C(OH)(CH₃)CH₃, or —CH₂CH₂F; (iv) R^{a} and R^{b}, or R^{Y} and R^{b}, are not joined as an unsubstituted morpholino or

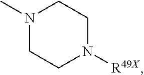

where R^{49X} is methyl, ethyl, methoxy, —C(O)CH₃, —CH₂CH₂OH, —CH₂CH₂OCH₃, —CH₂CH(OH)CH₃, —CH₂C(OH)(CH₃)CH₃, or —CH₂CH₂F; (v) R^{a} and R^{b}, or R^{Y} and R^{b}, are not joined as an unsubstituted piperazinyl, or substituted piperazinyl, or unsubstituted morpholinyl or substituted morpholinyl; (vi) R^{a} and R^{b}, or R^{Y} and R^{b}, are not joined as a group containing an unsubstituted piperazinyl, or substituted piperazinyl, or unsubstituted morpholinyl or substituted morpholinyl; (vii) R^{a} and R^{b}, or R^{Y} and R^{b}, are not joined as an unsubstituted heterocycloalkyl or substituted heterocycloalkyl; (viii) R^{a} and R^{b}, or R^{Y} and R^{b}, are not joined as a group containing an unsubstituted heterocycloalkyl or substituted heterocycloalkyl; (ix) R² is not unsubstituted morpholino or

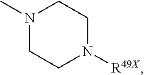

where R^{49X} is methyl, ethyl, methoxy, —C(O)CH₃, —CH₂CH₂OH, —CH₂CH₂OCH₃, —CH₂CH(OH)CH₃, —CH₂C(OH)(CH₃)CH₃, or —CH₂CH₂F; (x) R² is not a substituted alkyl substituted by a group defined in (ix); (xi) R² is not (a) an unsubstituted piperazinyl, or substituted piperazinyl, or unsubstituted morpholinyl or substituted morpholinyl; or (b) an alkyl substituted by a group defined in (xi)(a); (xii) R² is not a group containing an unsubstituted piperazinyl, or substituted piperazinyl, or unsubstituted morpholinyl or substituted morpholinyl; (xiii) R² is not an unsubstituted heterocycloalkyl or substituted heterocycloalkyl; (xiv) R² is not a group containing an unsubstituted heterocycloalkyl or substituted heterocycloalkyl; (xv) R^{b} in —CH₂—N(R^{b})R^{Y} is hydrogen and R^{Y} is not methyl or is not

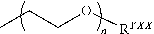

where n is an integer of 1 to 10; and R^{YXX} is hydrogen, optionally substituted alkyl or optionally substituted amidoalkyl; (xvi) for —CH₂—N(R^{b})R^{Y}, R^{b} and R^{Y} each is not methyl; (xvii) for —N(R^{a})R^{b}, R^{a} and R^{b} each is not methyl; (xviii) for —N(R^{a})R^{b}, R^{b} is not unsubstituted C1-C4 alkyl or is not substituted C1-C4 alkyl; and/or (xix) R is not an unsubstituted phenyl or substituted phenyl.

In certain embodiments, a compound is provided with the proviso that (i) Y or R² each independently is not

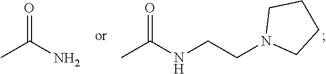

(ii) Y or R² each independently is not —C(O)N(H)R^{50X}, where R^{50X} is hydrogen, unsubstituted alkyl, substituted alkyl, unsubstituted heterocycloalkylalkyl or substituted heterocycloalkyl; (iii) Y or R² each independently is not hydrogen; (iv) Y or R² each independently is not chloro; (v) Y or R² each independently is not fluoro; (vi) Y or R² each independently is not halo; (vii) Y or R² each independently is not methoxy; (viii) Y or R² each independently is not unsubstituted alkoxy; (ix) Y or R² each independently is not cyano; (x) R¹ is not fluoro; (xi) R¹ is not halo; (xii) R⁴ is not fluoro; (xiii) R⁴ is not halo; (xiv) R³ is not fluoro; and/or (xvi) R³ is not halo.

In certain embodiments, a compound is provided with the proviso that: (i) R² is not an unsubstituted piperazinyl, or substituted piperazinyl, or unsubstituted morpholinyl or substituted morpholinyl; (ii) R² is not an unsubstituted heterocycloalkyl or substituted heterocycloalkyl; (iii) R² does not contain an unsubstituted heterocycloalkyl or substituted heterocycloalkyl; and/or (iv) R² is hydrogen, unsubstituted C1-C4 alkyl, ethyl or methyl.

In certain embodiments, a compound is provided where Y is —N(R^{b})C(O)—R^{Y} or —N(R^{b})C(O)—R^{V}—R^{Y} and R^{Y} is an optionally substituted alkenyl, with the proviso that (i) Y, R¹, R², R³ or R⁴ each independently is not one of the following designated Group A electrophilic groups:

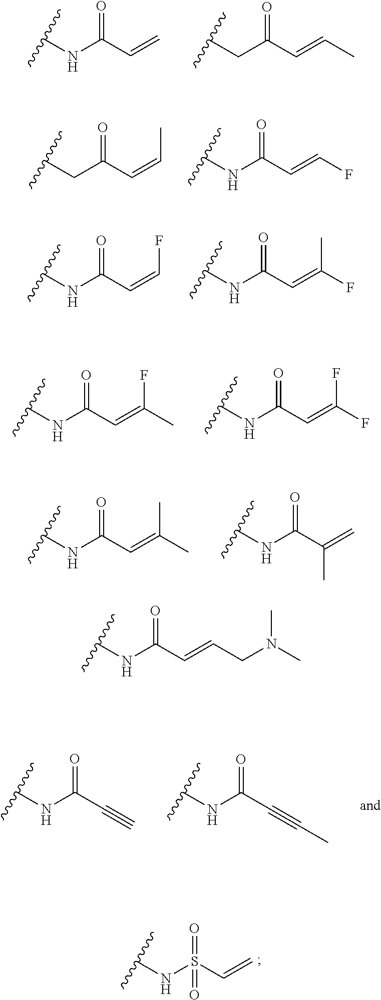

or (ii) Y, R¹, R², R³ or R⁴ each independently is not one of the following designated Group B electrophilic groups:

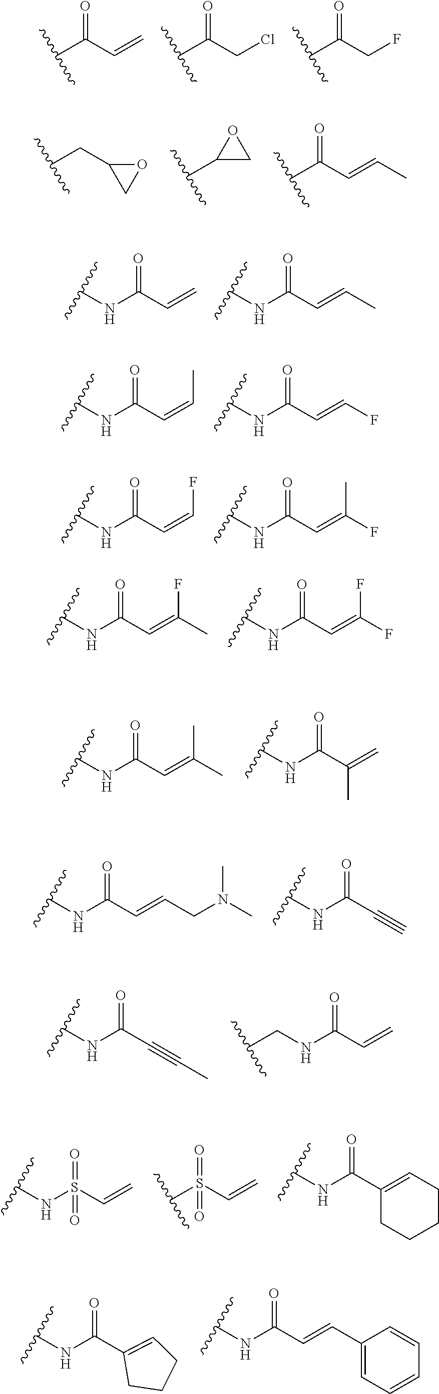

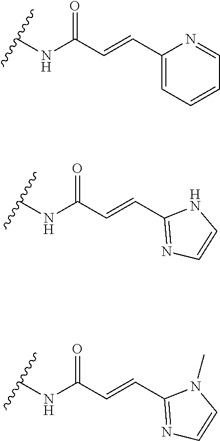 [SMILES: *NC(=O)/C=C/c1ccccn1 ; *NC(=O)/C=C/c1ncc[nH]1 ; *NC(=O)/C=C/c1nccn1C]

or (iii) Y, R¹, R², R³ or R⁴ each independently is not an electrophilic group capable of forming a covalent bond with a cysteine of a protein.

In certain embodiments, a compound is provided, where Y is —N(R^{b})C(O)—R^{Y} or —N(R^{b})C(O)—R^{V}—R^{Y}, and R^{Y} is —CH═CH₂, or Y, R¹, R³ or R⁴ is one of the designated Group A electrophilic groups, one of the designated Group B electrophilic groups, or an electrophilic group capable of forming a covalent bond with a cysteine of a protein, with the proviso that: (i) R² is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 mercaptoalkyl, R^{c}C(O)N(R^{d})—, —C(O)N(R^{c}R^{d}), —C(O)R^{z}, —C(O)OH, —C(O)OR^{u}, —B(OH)₂, hydroxy, halo, nitro or cyano, where R^{c}, R^{d} and R^{u} each independently is hydrogen or optionally substituted C1-C6 alkyl, optionally substituted C1-C4 alkyl, butyl, tert-butyl, iso-butyl, propyl, iso-propyl, ethyl or methyl and where R^{u} is an optionally substituted C1-C6 alkyl, optionally substituted C1-C4 alkyl, butyl, tert-butyl, iso-butyl, propyl, iso-propyl, ethyl or methyl; (ii) R² is hydrogen, optionally substituted C1-C4 alkyl, optionally substituted C1-C4 deuteroalkyl, optionally substituted C1-C4 haloalkyl, optionally substituted C1-C4 alkylamino or halo; (iii) R² is hydrogen, unsubstituted C1-C4 alkyl, unsubstituted C1-C4 deuteroalkyl, unsubstituted C1-C4 haloalkyl, or halo; (iv) R² is unsubstituted C1-C4 alkoxy, ethoxy or methoxy; and/or (v) R² is unsubstituted C1-C4 alkyl or methyl.

In certain embodiments, a compound is provided where Y is —N(R^{b})C(O)—R^{Y} or —N(R^{b})C(O)—R^{V}—R^{Y}, and R^{Y} is —CH═CH₂, or Y, R¹, R³ or R⁴ is one of the designated Group A electrophilic groups, one of the designated Group B electrophilic groups, or an electrophilic group capable of forming a covalent bond with a cysteine of a protein, with the proviso that:
(i) R² is not —NR^{a}R^{b}, where R^{a} and R^{b} are joined as an unsubstituted morpholino, substituted piperazinyl or substituted azetidinyl; (ii) R² is not —NR^{a}R^{b}, where R^{a} and R^{b} are joined as a substituted heterocycloalkyl or unsubstituted heterocycloalkyl; (iii) R² is not —NR^{a}R^{b}, where R^{a} is —CH₂CH₂N(CH₃)CH₃ and R^{b} is hydrogen or methyl; (iv) R² is not —NR^{a}R^{b}, where R^{a} is unsubstituted aminoalkyl or substituted aminoalkyl and R^{b} is hydrogen or unsubstituted alkyl; (v) R² is not —O—R^{51X}, where R^{51X} is —CH₂CH₂N(CH₃)CH₃, —CH₂CH₂OCH₃, substituted piperidinyl, substituted pyrrolidinyl or substituted azetidinyl; (vi) R² is not —O—R^{51X}, where R^{51X} is unsubstituted aminoalkyl or substituted aminoalkyl, unsubstituted alkoxyalkyl or substituted alkoxyalkyl, unsubstituted heterocycloalkyl or substituted hetercycloalkyl; (vii) R⁴ is not methoxy or fluoro; (viii) R⁴ is not unsubstituted alkoxy or halo; (ix) R¹⁷ and R¹⁸ are not fluoro or chloro; and/or (x) R¹¹ and R¹⁸ are not halo.

In certain embodiments, a compound is provided, where R¹, R², R³ or R⁴ is one of the designated Group A electrophilic groups, one of the designated Group B electrophilic groups, or an electrophilic group capable of forming a covalent bond with a cysteine of a protein, with the proviso that: (i) where Y is —NR^{a}R^{b}, R^{a} and R^{b} are not joined as an unsubstituted morpholino, substituted piperazinyl or substituted azetidinyl; (ii) where Y is —NR^{a}R^{b}, where R^{a} and R^{b} are not joined as a substituted heterocycloalkyl or unsubstituted heterocycloalkyl; (iii) where Y is —NR^{a}R^{b}, R^{a} is not —CH₂CH₂N(CH₃)CH₃ and R^{b} is not hydrogen or methyl; (iv) where Y is —NR^{a}R^{b}, R^{a} is not unsubstituted aminoalkyl or substituted aminoalkyl and R^{b} is not hydrogen or unsubstituted alkyl; (v) Y is not —O—R^{51X}, where R^{51X} is —CH₂CH₂N(CH₃)CH₃, —CH₂CH₂OCH₃, substituted piperidinyl, substituted pyrrolidinyl or substituted azetidinyl; (vi) Y is not —O—R^{51X}, where R^{51X} is unsubstituted aminoalkyl or substituted aminoalkyl, unsubstituted alkoxyalkyl or substituted alkoxyalkyl, or unsubstituted heterocycloalkyl or substituted heterocycloalkyl; (vii) R⁴ is not methoxy or fluoro; (viii) R⁴ is not unsubstituted alkoxy or halo; (ix) R¹⁷ and R¹⁸ are not fluoro or chloro; and/or (x) R¹⁷ and R¹⁸ are not halo.

In certain embodiments, a compound is provided, where at least one of R¹⁰, R¹¹, R¹² or R¹³ is R^{W} or —W—R^{W}, with the proviso that R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{20A}, R²¹, R^{21A}, R²², R^{22A}, R^{14B}, R^{15B}, R^{16B}, R^{17B}, R^{18B}, R^{19B}, R^{20B}, R^{21B} and R^{22B} each independently is (i) not

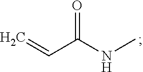

(ii) not one of the designated Group A electrophilic groups, (iii) not one of the designated Group B electrophilic groups, and/or (iv) not an electrophilic group capable of forming a covalent bond with a cysteine of a protein.

In the context of a chemical structure, a reference to a chemical structure, or a structure provided as part of a synthetic scheme, a number or letter normally designated as a superscript, for example, the “1” in R¹, or the “L” in R^{L}, may be referred to as a subscript, for example, R₁ or R_{L}, or without any modification of script, such as, for example, R1 or RL.

Any definition herein may be used in combination with any other definition to describe a composite structural group. By convention, the trailing element of any such definition is that which attaches to a parent moiety. The composite group alkylamido, for example, would represent an alkyl group attached to a parent molecule through an amido group, the term amidoalkyl would represent an amido group attached to a parent molecule through an alkyl group, the term alkylalkoxy would represent an alkyl group attached to a parent molecule through an alkoxy group, and the term alkoxyalkyl would represent an alkoxy group attached to a parent molecule through an alkyl group, for example.

When a group is defined to be “null,” the group is absent. The term “optionally substituted” means the anteceding group may be substituted or unsubstituted. The term “substituted,” as used herein, refers, without limitation, to one or more substituents that can include, for example, substituents independently selected from the following groups or a particular designated set of groups, alone or in combination: lower alkyl, lower alkenyl, lower alkynyl, lower alkanoyl, lower heteroalkyl, lower aryl, lower cycloalkyl, lower heteroaryl, lower heterocycloalkyl, aryl, cycloalkyl, heteroaryl, heterocycloalkyl, lower haloalkyl, lower haloalkenyl, lower haloalkynyl, lower perhaloalkyl, lower perhaloalkoxy, phenyl, aryloxy, lower hydroxyalkyl, lower mercaptoalkyl, lower aminoalkyl, lower arylaminoalkyl, aryloxyalkyl, lower aryloxyalkyl, arylthioalkyl, lower arylthioalkyl, heteroarylaminoalkyl, heteroaryloxyalkyl, heteroarylthioalkyl, arylalkyl, lower arylalkyl, heteroarylalkyl, lower heteroarylalkyl, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, B(OH)₂, pyridinyl, thiophene, furanyl, lower carbamate, and lower urea. Two substituents may be joined together to form a fused five-, six-, or seven-membered carbocyclic, heterocyclic aryl, or heteroaryl ring system having zero to three heteroatoms, for example, forming methylenedioxy or ethylenedioxy. An optionally substituted group may contain a deuterium in place of one or more hydrogen atoms (for example, —CD₃ instead of —CH₃). An optionally substituted group may be unsubstituted (for example, —CH₂CH₃), fully substituted (for example, —CF₂CF₃), monosubstituted (for example, —CH₂CH₂F) or substituted at a level anywhere in-between fully substituted and monosubstituted (for example, —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed. An optional substitution often is as defined, sometimes immediately following the phrase, “optionally substituted with.”

The term R or the term R′, appearing by itself and without a number designation, unless otherwise defined, refers to a moiety chosen from hydrogen (H), alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (for example aryl, heterocycle, R, etc.) occur more than one time in a formula or generic structure, its definition at each occurrence is independent of the definition at every other occurrence. Certain groups may be attached to a parent molecule or may occupy a position in a chain of elements from either end as written. Thus, by way of example only, an asymmetrical group such as —C(O)N(R)— may be attached to a parent moiety at either the carbon or the nitrogen.

In embodiments, the term ‘substituted’ and ‘substituent group’, as used herein, means a group selected from the following moieties:
(A) oxo, halogen, —CCl₃, —CBr₃, —CF₃, —Cl₃, —CHCl₂, —CHBr₂, —CHF₂, —CHl₂, —CH₂Cl, —CH₂Br, —CH₂F, —CH₂I, —OCCl₃, —OCF₃, —OCBr₃, —OCl₃, —OCHCl₂, —OCHBr₂, —OCHl₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (for example, C₁-C₈ alkyl, C₁-C₆ alkyl, or C₁-C₄ alkyl), unsubstituted heteroalkyl (for example, 2 to 8 membered heteroalkyl, 2 to 6 membered heteroalkyl, or 2 to 4 membered heteroalkyl), unsubstituted cycloalkyl (for example, C₃-C₈ cycloalkyl, C₃-C₆ cycloalkyl, or C₅-C₆ cycloalkyl), unsubstituted heterocycloalkyl (for example, 3 to 8 membered heterocycloalkyl, 3 to 6 membered heterocycloalkyl, or 5 to 6 membered heterocycloalkyl), unsubstituted aryl (for example, C₆-C₁₀ aryl, C₁₀ aryl, or phenyl), or unsubstituted heteroaryl (for example, 5 to 10 membered heteroaryl, 5 to 9 membered heteroaryl, or 5 to 6 membered heteroaryl), and
(B) alkyl (for example, C₁-C₈ alkyl, C₁-C₆ alkyl, or C₁-C₄ alkyl), heteroalkyl (for example, 2 to 8 membered heteroalkyl, 2 to 6 membered heteroalkyl, or 2 to 4 membered heteroalkyl), cycloalkyl (for example, C₃-C₈ cycloalkyl, C₃-C₆ cycloalkyl, or C₅-C₆ cycloalkyl), heterocycloalkyl (for example, 3 to 8 membered heterocycloalkyl, 3 to 6 membered heterocycloalkyl, or 5 to 6 membered heterocycloalkyl), aryl (for example, C₆-C₁₀ aryl, C₁₀ aryl, or phenyl), heteroaryl (for example, 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —Cl₃, —CHCl₂, —CHBr₂, —CHF₂, —CHl₂, —CH₂Cl, —CH₂Br, —CH₂F, —CH₂I, —OCCl₃, —OCF₃, —OCBr₃, —OCl₃, —OCHCl₂, —OCHBr₂, —OCHl₂, —OCHF₂, —OCH₂Cl, —OCH₂Br, —OCH₂I, —OCH₂F, —CN, —OH, —NH₂, —OOOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (for example, C₁-C₈ alkyl, C₁-C₆ alkyl, or C₁-C₄ alkyl), unsubstituted heteroalkyl (for example, 2 to 8 membered heteroalkyl, 2 to 6 membered heteroalkyl, or 2 to 4 membered heteroalkyl), unsubstituted cycloalkyl (for example, C₃-C₈ cycloalkyl, C₃-C₆ cycloalkyl, or C₅-C₆ cycloalkyl), unsubstituted heterocycloalkyl (for example, 3 to 8 membered heterocycloalkyl, 3 to 6 membered heterocycloalkyl, or 5 to 6 membered heterocycloalkyl), unsubstituted aryl (for example, C₆-C₁₀ aryl, C₁₀ aryl, or phenyl), or unsubstituted heteroaryl (for example, 5 to 10 membered heteroaryl, 5 to 9 membered heteroaryl, or 5 to 6 membered heteroaryl), and
(ii) alkyl (for example, C₁-C₈ alkyl, C₁-C₆ alkyl, or C₁-C₄ alkyl), heteroalkyl (for example, 2 to 8 membered heteroalkyl, 2 to 6 membered heteroalkyl, or 2 to 4 membered heteroalkyl), cycloalkyl (for example, C₃-C₈ cycloalkyl, C₃-C₆ cycloalkyl, or C₅-C₆ cycloalkyl), heterocycloalkyl (for example, 3 to 8 membered heterocycloalkyl, 3 to 6 membered heterocycloalkyl, or 5 to 6 membered heterocycloalkyl), aryl (for example, C₆-C₁₀ aryl, C₁₀ aryl, or phenyl), heteroaryl (for example, 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —Cl₃, —CHCl₂, —CHBr₂, —CHF₂, —CHl₂, —CH₂Cl, —CH₂Br, —CH₂F, —CH₂I, —OCCl₃, —OCF₃, —OCBr₃, —OCl₃, —OCHCl₂, —OCHBr₂, —OCHl₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (for example, C₁-C₈ alkyl, C₁-C₆ alkyl, or C₁-C₄ alkyl), unsubstituted heteroalkyl (for example, 2 to 8 membered heteroalkyl, 2 to 6 membered heteroalkyl, or 2 to 4 membered heteroalkyl), unsubstituted cycloalkyl (for example, C₃-C₈ cycloalkyl, C₃-C₆ cycloalkyl, or C₅-C₆ cycloalkyl), unsubstituted heterocycloalkyl (for example, 3 to 8 membered heterocycloalkyl, 3 to 6 membered heterocycloalkyl, or 5 to 6 membered heterocycloalkyl), unsubstituted aryl (for example, C₆-C₁₀ aryl, C₁₀ aryl, or phenyl), or unsubstituted heteroaryl (for example, 5 to 10 membered heteroaryl, 5 to 9 membered heteroaryl, or 5 to 6 membered heteroaryl), and
(b) alkyl (for example, C₁-C₈ alkyl, C₁-C₆ alkyl, or C₁-C₄ alkyl), heteroalkyl (for example, 2 to 8 membered heteroalkyl, 2 to 6 membered heteroalkyl, or 2 to 4 membered heteroalkyl), cycloalkyl (for example, C₃-C₈ cycloalkyl, C₃-C₆ cycloalkyl, or C5-C6 cycloalkyl), heterocycloalkyl (for example, 3 to 8 membered heterocycloalkyl, 3 to 6 membered heterocycloalkyl, or 5 to 6 membered heterocycloalkyl), aryl (for example, C₆-C₁₀ aryl, C₁₀ aryl, or phenyl), heteroaryl (for example, 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CHCl₂, —CHBr₂, —CHF₂, —CHl₂, —CH₂Cl, —CH₂Br, —CH₂F, —CH₂I, —OCCl₃, —OCF₃, —OCBr₃, —OCl₃, —OCHCl₂, —OCHBr₂, —OCHl₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (for example, C₁-C₈ alkyl, C₁-C₆ alkyl, or C₁-C₄ alkyl), unsubstituted heteroalkyl (for example, 2 to 8 membered heteroalkyl, 2 to 6 membered heteroalkyl, or 2 to 4 membered heteroalkyl), unsubstituted cycloalkyl (for example, C₃-C₈ cycloalkyl, C₃-C₆ cycloalkyl, or C₅-C₆ cycloalkyl), unsubstituted heterocycloalkyl (for example, 3 to 8 membered heterocycloalkyl, 3 to 6 membered heterocycloalkyl, or 5 to 6 membered heterocycloalkyl), unsubstituted aryl (for example, C₆-C₁₀ aryl, C₁₀ aryl, or phenyl), or unsubstituted heteroaryl (for example, 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In some embodiments, at least one or all of these groups are substituted with at least one size-limited substituent group. In some embodiments, at least one or all of these groups are substituted with at least one lower substituent group.

In some embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (for example, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (for example, is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (for example, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (for example, is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (for example, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (for example, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (for example, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (for example, substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. Non-limiting examples of acyl groups include formyl, alkanoyl and aroyl.

An “acetyl” group refers to a —C(O)CH₃ group.

The term “aliphatic,” as used herein, refers to saturated and partially unsaturated, nonaromatic, straight chain (i.e., unbranched), branched and cyclic (including bicyclic and polycyclic) hydrocarbons which may be optionally substituted with one or more functional groups. In certain embodiments, an aliphatic group contains 1 to 20 carbon atoms, 1 to 10 carbon atoms, 1 to 8 carbon atoms, 1 to 6 carbon atoms or 1 to 3 carbon atoms.

An “alkylcarbonyl” or “alkanoyl” group refers to an alkyl group attached to a parent molecular moiety through a carbonyl group. Non-limiting examples of such groups include methylcarbonyl and ethylcarbonyl.

The term “alkenyl,” as used herein, alone or in combination, refers to a straight-chain or branched-chain hydrocarbon radical having one or more double bonds and containing from 2 to 20 carbon atoms. In certain embodiments, an alkenyl includes 2 to 6 carbon atoms. The term “alkenylene” refers to a carbon-carbon double bond system attached at two or more positions such as ethenylene [(—CH═CH—),(—C::C—)]. Non-limiting examples of alkenyl radicals include ethenyl, propenyl, 2-methylpropenyl, 1,4-butadienyl and the like.

Unless otherwise specified, the term “alkenyl” may include “alkenylene” groups. An alkene functionality is not directly bonded to a nitrogen. An alkenyl group containing an alkene functionality and alkyl portion, such as an allyl group, for example, can be bonded to a nitrogen such that the alkene functionality is not directly bonded to the nitrogen.

The term “alkoxy,” as used herein, alone or in combination, refers to an alkyl ether radical, where the term alkyl is as defined below. Non-limiting examples of alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a saturated straight-chain or branched-chain hydrocarbon radical containing from 1 to 20 carbon atoms. The term “straight-chain alkyl” refers to a saturated straight-chain hydrocarbon radical. The term “branched-chain alkyl” refers to a saturated branched-chain hydrocarbon radical. In certain embodiments, an alkyl includes 1 to 10 carbon atoms (C1-C10 alkyl), 1 to 8 carbon atoms (C1-C8 alkyl), 1 to 6 carbon atoms (C1-C6 alkyl) or 1 to 3 carbon atoms (C1-C3 alkyl). Alkyl groups may be optionally substituted as defined herein. Non-limiting examples of alkyl radicals include methyl, ethyl, n-propyl, isopropyl, n-butyl, isobutyl, sec-butyl, tert-butyl, pentyl, iso-amyl, hexyl, octyl, nonyl and the like.

The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to a parent molecular moiety through an amino group. Alkylamino groups include monoalkylated groups (monoalkylamino) or dialkylated groups (dialkylamino), non-limiting examples of which include N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical where the term alkyl is as defined above and where the sulfur may be singly or doubly oxidized. Non-limiting examples of alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, an alkynyl includes 2 to 6 carbon atoms. In some embodiments, an alkynyl includes 2 to 4 carbon atoms. The term “alkynylene” refers to a carbon-carbon triple bond attached at two positions such as ethynylene (—C:::C—, —C≡C—). Non-limiting examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups. An alkyne functionality is not directly bonded to nitrogen. An alkynyl group containing an alkyne functionality and an alkyl portion, such as a propargyl group, for example, can be bonded to a nitrogen such that the alkyne functionality is not directly bonded to the nitrogen.

The terms “amido” and “carbamoyl,” as used herein, alone or in combination, refer to an amino group as described below attached to a parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated.

The term “acylamino” as used herein, alone or in combination, includes an acyl group attached to a parent moiety through an amino group. A non-limiting example of an “acylamino” group is acetylamino (CH₃C(O)NH—).

The term “amino,” as used herein, alone or in combination, refers to —NRR′, where R and R′ are independently chosen from hydrogen, alkyl, alkenyl, alkynyl, acyl, heteroalkyl, aryl, cycloalkyl, heteroaryl, and heterocycloalkyl, any of which may themselves be optionally substituted. Additionally, R and R′ may combine to form heterocycloalkyl or heteroaryl, either of which may be optionally substituted.

The term “aminoalkyl,” as used herein, refers to an amino group attached to a parent molecule through an alkyl group (N(R)(R′)-alkyl-), where R and R′ are defined herein. The term “lower aminoalkyl,” as used herein, refers to an amino group attached to a parent molecule through a lower alkyl group (N(R)(R′)-lower alkyl-), where “lower alkyl,” R and R′ are defined herein.

The term “aryl,” as used herein, alone or in combination, refers to an aromatic cyclic ring system, or aromatic hydrocarbon ring system, in which all of the atoms that form the covalent structure of the one or more aromatic rings are carbon (referred to herein as an “aryl ring”). The aryl ring may be optionally substituted as defined herein. The ring system may be monocyclic or fused polycyclic, for example, bicyclic or tricyclic (containing two or three rings fused together). In certain embodiments, the monocyclic aryl ring is C4-C10, or C5-C9, or C5-C8, or C5-C7, or, in certain embodiments, C5-C6, where these carbon numbers refer to the number of carbon ring member atoms that form the ring system. In some embodiments, the polycyclic ring system is a bicyclic aryl group, where the bicyclic aryl group in some embodiments is C8-C12, or, for example, C9-C10. In some embodiments, the polycyclic ring system is a tricyclic aryl group, where the tricyclic aryl group is C11-C18, or, for example, C12-C16. Non-limiting examples of aryl ring systems include phenyl (monocyclic, C6), naphthyl (bicyclic, C10), anthracenyl (tricyclic, C14) and phenanthryl (tricyclic, C14).

The term “arylalkenyl” or “aralkenyl,” as used herein, alone or in combination, refers to an aryl group attached to a parent molecular moiety through an alkenyl group.

The term “arylalkoxy” or “aralkoxy,” as used herein, alone or in combination, refers to an aryl group attached to a parent molecular moiety through an alkoxy group.

The term “arylalkyl” or “aralkyl,” as used herein, alone or in combination, refers to an aryl group attached to a parent molecular moiety through an alkyl group. The term “lower arylalkyl” or “lower aralkyl,” as used herein, alone or in combination, refers to a lower aryl group attached to a parent molecular moiety through a lower alkyl group, where “lower aryl” and “lower alkyl” are as defined herein.

The term “arylalkynyl” or “aralkynyl,” as used herein, alone or in combination, refers to an aryl group attached to a parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid, non-limiting examples of which include benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term “arylaminoalkyl” as used herein refers to an aryl group attached to a parent molecule through an aminoalkyl group (aryl-N(R)-alkyl-), where R is as defined herein. The term “lower arylaminoalkyl” as used herein refers to a lower aryl group attached to a parent molecule through a lower aminoalkyl group (lower aryl-N(R)-lower alkyl-), where “lower aryl,” “lower aminoalkyl” and R are as defined herein.

The term “aryloxy” as used herein, alone or in combination, refers to an aryl group attached to a parent molecular moiety through an oxygen atom.

The term “aryloxyalkyl” as used herein refers to an aryl group attached to a parent molecule through an alkyl ether group (aryl-O-alkyl-). The term “lower aryloxyalkyl” as used herein refers to a lower aryl group attached to a parent molecule through a lower alkyl ether group (lower aryl-O-lower alkyl-), where “lower aryl” and “lower alkyl” are defined herein.

The term “arylthioalkyl” as used herein refers to an aryl group attached to a parent molecule through a thioalkyl group (aryl-S-alkyl-). The term “lower arylthioalkyl” as used herein refers to a lower aryl group attached to a parent molecule through a lower thioalkyl group (lower aryl-S-lower alkyl-) where “lower aryl” and “lower alkyl” are defined herein.

The terms “benzo” and “benz,” as used herein, alone or in combination, refer to the divalent radical C₆H₄═ derived from benzene. Non-limiting examples include benzothiophene and benzimidazole.

The term “boronic acid” as used herein refers to a —B(OH)₂ group.

The term “carbamate,” as used herein, alone or in combination, refers to an ester of carbamic acid (—NHCOO—) which may be attached to a parent molecular moiety from either the nitrogen or acid end, and which may be optionally substituted as defined herein.

The term “O-carbamyl” as used herein, alone or in combination, refers to a —OC(O)NRR′ group where R and R′ are as defined herein.

The term “N-carbamyl” as used herein, alone or in combination, refers to a ROC(O)NR′— group, where R and R′ are defined herein.

The term “carbonyl,” as used herein, when alone includes formyl [—C(O)H] and in combination includes a —C(O)— group.

The term “carboxyl” or “carboxy,” as used herein, refers to —C(O)OH or the corresponding “carboxylate” anion (for example, in a carboxylic acid salt). An “O-carboxy” group refers to a RC(O)O— group, where R is as defined herein. A “C-carboxy” group refers to a —C(O)OR group where R is as defined herein.

The term “cyano,” as used herein, alone or in combination, refers to —CN.

The terms “cycloalkyl,” and, interchangeably, “carbocycle,” as used herein, alone or in combination, refers to a ring system in which all of the ring member atoms are carbon and at least one of the rings is a saturated or partially unsaturated aliphatic cyclic ring moiety (referred to herein as a “cycloalkyl ring” or “carbocycle ring”). In some embodiments, each cyclic moiety contains from 3 to 12 carbon ring member atoms which may be optionally substituted as defined herein. In some embodiments, a cycloalkyl group contains 3 to 10 carbon ring member atoms. In certain embodiments, a cycloalkyl includes 5 to 7 carbon atoms. In certain embodiments, a cycloalkyl includes 5 to 6 carbon atoms. A cycloalkyl can be a monocyclic or polycyclic, for example, bicyclic or tricyclic, ring system in which at least one cyclic ring is a cycloalkyl ring. In certain embodiments, the monocyclic cycloalkyl ring is C3-C10, or C5-C9, or C5-C8, or C5-C7, or, in certain embodiments, C5-C6, where these carbon numbers refer to the number of carbon ring member atoms that form the ring system. Polycyclic cycloalkyl ring systems include fused, bridged and spiro-fused rings.

Polycyclic cycloalkyl ring systems as defined herein, include ring systems in which one or more cycloalkyl rings is/are fused to one or more aryl rings (benzo-fused cycloalkyl ring systems) and/or other cycloalkyl rings. In some embodiments, all of the rings in a polycyclic cycloalkyl ring system are cycloalkyl rings. In some embodiments, the polycyclic ring system is a bicyclic cycloalkyl group, where the bicyclic cycloalkyl group in some embodiments is C8-C12, or, for example, C9-C10. In some embodiments, the polycyclic ring system is a tricyclic cycloalkyl group, where the tricyclic cycloalkyl group is C11-C18, or, for example, C12-C16. Non-limiting examples of such cycloalkyl groups include cyclopropyl, cyclobutyl, cyclopentyl, cyclohexyl, cycloheptyl, adamantyl, octahydronaphthalene, decahydronaphthalene, bicyclo[1,1,1]pentane and the like.

Examples of aryl-fused cyclolalkyl ring systems include a benzene ring fused to hydrogenated or partially hydrogenated ring systems, non-limiting examples of which include dihydronaphthalene, tetrahydronaphthalene and indanyl. In polycyclic systems in which a cycloalkyl is fused to an aryl, attachment of the polycycle to the indicated point of attachment on the parent molecule may be through any ring atom of the polycycle rings. In some embodiments of polycyclic cycloalkyls, the polycycle is attached to the indicated point of attachment through a ring member atom of a cycloalkyl ring. In some embodiments of polycyclic cycloalkyls, the polycycle is attached to the indicated point of attachment through a ring member atom of a ring that is not a cycloalkyl ring, for example, an aryl ring.

The term “carbocycle-alkyl” or “cycloalkylalkyl” as used herein, alone or in combination, refers to a carbocycle group attached to a parent molecular moiety through an alkyl group.

The term “deuteroalkyl,” as used herein, alone or in combination, refers to an alkyl radical having the meaning as defined herein where one or more or all hydrogens are replaced with a deuterium. Specifically included are monodeuteroalkyl, dideuteroalkyl, trideuteroalkyl and polydeuteroalkyl radicals. A “lower deuteroalkyl” group is a C1-C6, C1-C5, C1-C4, C1-C3 or C1-C2 alkyl in which one or more hydrogens are replaced with a deuterium. A non-limiting example of a lower deuteroalkyl group is the trideuteroalkyl —CD₃, in which the three hydrogens of —CH₃ are replaced by deuterium.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to a parent molecular moiety through an oxygen atom.

The term “haloalkyl,” as used herein, alone or in combination, refers to an alkyl radical having the meaning as defined above where one or more hydrogens are replaced with a halogen. Specifically included are monohaloalkyl, dihaloalkyl and polyhaloalkyl radicals. A monohaloalkyl radical, for example, sometimes include an iodo, bromo, chloro or fluoro atom within the radical. Dihalo and polyhaloalkyl radicals sometimes include two or more of the same halo atoms or a combination of different halo radicals. Non-limiting examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Non-limiting examples include fluoromethylene (—CFH—), difluoromethylene (—CF₂—), chloromethylene (—CHCl—) and the like.

The term “heteroaliphatic,” as used herein, refers to an aliphatic moiety, as defined herein, that contains one or more heteroatoms, such as, for example, oxygen, nitrogen, sulfur, phosphorous and/or silicon, for example, in place of a carbon atom or between carbon atoms. In some embodiments, a heteroaliphatic group contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroaliphatic group. In some embodiments, up to two heteroatoms may be consecutive. In certain embodiments, a heteroaliphatic group includes 2 to 20 carbon atoms, 2 to 10 carbon atoms, 2 to 8 carbon atoms or 2 to 6 carbon atoms.

The term “heteroalkyl,” as used herein, alone or in combination, refers to a saturated or unsaturated, stable straight or branched hydrocarbon chain having the stated number of carbon atoms and one or more heteroatoms, such as, for example, oxygen, nitrogen, sulfur, phosphorous and/or silicon, for example, in place of a carbon atom. In some embodiments, a heteroalkyl contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroalkyl group. In some embodiments, up to two heteroatoms may be consecutive, such as, for example, —CH₂—NH—OCH₃. In certain embodiments, a heteroalkyl includes 2 to 20 carbon atoms, 2 to 10 carbon atoms, 2 to 8 carbon atoms or 2 to 6 carbon atoms. In some instances, a heteroalkyl contains from 1 to 3 degrees of unsaturation. Heteroalkyl groups may be optionally substituted as defined herein.

The term “heteroalkenyl,” as used herein, alone or in combination, refers to an alkenyl moiety, as defined herein, that contains one or more heteroatoms, such as, for example, oxygen, nitrogen, sulfur, phosphorous and/or silicon, for example, in place of a carbon atom or between carbon atoms. In some embodiments, a heteroalkenyl contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroalkenyl group. In some embodiments, up to two heteroatoms may be consecutive. In certain embodiments, a heteroalkenyl includes 2 to 20 carbon atoms, 2 to 10 carbon atoms, 2 to 8 carbon atoms or 2 to 6 carbon atoms.

The term “heteroalkynyl,” as used herein, alone or in combination, refers to an alkynyl moiety, as defined herein, that contains one or more heteroatoms, such as, for example, oxygen, nitrogen, sulfur, phosphorous and/or silicon, for example, in place of a carbon atom or between carbon atoms. In some embodiments, a heteroalkynyl contains from one to three heteroatoms chosen from O, N, and S, and where the nitrogen and sulfur atoms may optionally be oxidized and the nitrogen heteroatom may optionally be quaternized. In certain embodiments, the heteroatom(s) may be placed at any interior position of the heteroalkynyl group. In some embodiments, up to two heteroatoms may be consecutive. In certain embodiments, a heteroalkynyl includes 2 to 20 carbon atoms, 2 to 10 carbon atoms, 2 to 8 carbon atoms or 2 to 6 carbon atoms.

The term “heteroaryl,” as used herein, alone or in combination, refers to a cyclic ring system in which at least one of the rings is an aromatic ring in which all ring member atoms are carbon, except for at least one heteroatom (referred to herein as a “heteroaryl ring”), such as, for example, nitrogen, oxygen and sulfur. The heteroaryl ring may be optionally substituted as defined herein. A heteroaryl can be a monocyclic or a fused polycyclic, for example, bicyclic or tricyclic, ring system in which at least one cyclic ring is an aromatic heteroaryl ring. Polycyclic, for example, bicyclic and tricyclic, fused heteroaryl ring systems as defined herein include heteroaryl ring systems in which one or more heteroaryl rings is/are fused to one or more aryl rings (which are referred to herein as aryl-fused heteroaryl rings), one or more cycloalkyl rings and/or one or more other heteroaryl rings. In some embodiments, all of the rings in a polycyclic heteroaryl ring system are heteroaryl rings. In certain embodiments, a heteroaryl ring contains at least one atom chosen from O, S, and N. In certain embodiments, a heteroaryl ring is a 3 to 15 membered monocyclic ring. In certain embodiments, a monocyclic heteroaryl group may contain from 4 to 10 ring member atoms, and may have, for example, 1 to 4 heteroatoms in the ring, where the remaining ring member atoms are carbon. In some embodiments, a bicyclic heteroaryl ring may contain from 8 to 15 ring member atoms, and have from 1 to 8 heteroatoms, where the remaining ring member atoms are carbon. In some embodiments, a tricyclic heteroaryl ring may contain from 11 to 18 ring member atoms, and have from 1 to 10 heteroatoms, where the remaining ring member atoms are carbon. Non-limiting examples of heteroaryls include pyrrolyl, pyrrolinyl, imidazolyl, pyrazolyl, pyridyl, pyrimidinyl, pyrazinyl, pyridazinyl, triazinyl, tetrazinyl, triazolyl, tetrazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, thienopyridinyl, furopyridinyl, pyrrolopyridinyl and the like. Exemplary bicyclic and tricyclic heteroaryl groups include phenanthrolinyl, dibenzofuranyl, acridinyl, phenanthridinyl, dihydro[1,3]oxazolo[4,5-b]pyridinyl, benzothiazolyl, and the like. In polycyclic systems in which a heteroaryl is fused to one or more rings that are not heteroaryl, attachment of the polycycle to the indicated point of attachment on the parent molecule may be through any ring member atom of the polycycle rings. In some embodiments of polycyclic heteroaryls, the polycycle is attached to the indicated point of attachment through a ring member atom of a heteroaryl ring. In some embodiments of monocyclic or polycyclic heteroaryls, the monocycle or polycycle is attached to the indicated point of attachment through a ring member heteroatom of a heteroaryl ring. In some embodiments of polycyclic heteroaryls, the polycycle is attached to the indicated point of attachment through a ring member atom of a ring that is not a heteroaryl ring, for example, an aryl ring or a cycloalkyl ring. “Heteroaryl” includes sulfones, sulfoxides, N-oxides of tertiary nitrogen ring member atoms, and carbocyclic fused and benzo-fused ring systems. Non-limiting examples of a heteroaryl group may be referred to as an aryl group having one or more carbon atoms substituted with O, NRⁿ, S, SO, SO₂, where “n” denotes any positive integer.

The term “heteroarylalkyl” as used herein, alone or in combination, refers to an unsubstituted or substituted heteroaryl group attached to a parent molecular moiety through an alkyl group. The term “lower heteroarylalkyl” as used herein, alone or in combination, refers to an unsubstituted or substituted lower heteroaryl group attached to a parent molecular moiety through a lower alkyl group where “lower heteroaryl” and “lower alkyl” are as defined herein.

The term “heteroarylaminoalkyl” as used herein refers to a heteroaryl group attached to a parent molecule through an aminoalkyl group (heteroaryl-N(R)-alkyl-), where R is as defined herein. The term “lower heteroarylaminoalkyl” as used herein refers to a lower heteroaryl group attached to a parent molecule through a lower aminoalkyl group (lower heteroaryl-N(R)-lower alkyl-), where “lower heteroaryl,” “lower alkyl” and R are as defined herein.

The term “heteroaryloxyalkyl” as used herein refers to a heteroaryl group attached to a parent molecule through an alkyl ether group (heteroaryl-O-alkyl-). The term “lower heteroaryloxyalkyl” as used herein refers to a lower heteroaryl group attached to a parent molecule through a lower alkyl ether group (lower heteroaryl-O-lower alkyl-), where “lower heteroaryl” and “lower alkyl” are defined herein.

The term “heteroarylthioalkyl” as used herein refers to a heteroaryl group attached to a parent molecule through a thioalkyl group (heteroaryl-S-alkyl-). The term “lower heteroarylthioalkyl” as used herein refers to a lower heteroaryl group attached to a parent molecule through a lower thioalkyl group (lower heteroaryl-S-lower alkyl-), where “lower heteroaryl” and “lower alkyl” are defined herein.

The term “heterocycle-alkyl” as used herein, alone or in combination, refers to a substituted or unsubstituted heterocycle group attached to a parent molecular moiety through an alkyl group.

The terms “heterocycloalkyl” and, interchangeably, “heterocycle,” or “heterocyclic” as used herein, alone or in combination, each refer to a ring system in which at least one of the rings is a saturated or partially unsaturated, heteroaliphatic, nonaromatic cyclic ring moiety in which all of the ring member atoms are carbon, except for at least one heteroatom (referred to herein as a “heterocycloalkyl ring,” “heterocycle ring” or “heterocyclic ring”). The one or more heteroatoms that can be in the ring include, for example, nitrogen, oxygen, sulfur, phosphorous and/or silicon. In some embodiments, the ring heteroatom or heteroatoms is selected from nitrogen, oxygen and sulfur. The heterocycloalkyl ring may be optionally substituted as defined herein. A heterocycloalkyl is a monocyclic or polycyclic, for example, bicyclic or tricyclic, ring system in which at least one cyclic ring is a heterocycloalkyl ring. Polycyclic heterocycloalkyl ring systems include fused, bridged and spiro-fused rings. Polycyclic heterocycloalkyl ring systems as defined herein, include ring systems in which one or more heterocycloalkyl rings is/are fused to one or more cycloalkyl, aryl, heteroaryl and/or heterocycloalkyl rings. In some embodiments, all of the rings in a polycyclic heterocycloalkyl ring system are heterocycloalkyl rings. In certain embodiments, a heterocycloalkyl includes 1 to 4 heteroatoms as ring member atoms. In some embodiments, a heterocycloalkyl moiety includes 1 to 2 heteroatoms as ring member atoms. In certain embodiments, a heterocycloalkyl moiety includes 3 to 8 ring member atoms in each ring. In some embodiments, a heterocycloalkyl moiety includes 3 to 7 ring member atoms in each ring. In yet some embodiments, a heterocycloalkyl moiety includes 5 to 6 ring member atoms in each ring. In some embodiments, a heterocycloalkyl can be a 3 to 15 membered nonaromatic ring, or a fused bicyclic, or tricyclic non-aromatic ring, which contains at least one atom chosen from O, S, and N. In certain embodiments, a monocyclic heterocycloalkyl or heterocycle group may contain from 4 to 10 ring member atoms, and may have, for example, 1 to 4 heteroatoms in the ring, where the remaining ring member atoms are carbon. In some embodiments, a bicyclic heterocycloalkyl or heterocycle group may contain from 8 to 15 ring member atoms, and have from 1 to 8 heteroatoms, where the remaining ring member atoms are carbon. In some embodiments, a tricyclic heterocycloalkyl or heterocycle group may contain from 11 to 18 ring member atoms, and have from 1 to 10 heteroatoms, where the remaining ring member atoms are carbon. The term also includes fused polycyclic groups where one or more heterocyclic rings are fused with one or more cycloalkyl rings, aryl, heteroaryl and/or other heterocyclic groups. In polycyclic systems in which a heterocycloalkyl ring is fused to one or more rings that are not heterocycloalkyl, attachment of the polycycle to the indicated point of attachment on the parent molecule may be through any ring member atom of the polycycle rings. In some embodiments of polycyclic heterocycloalkyls, the polycycle is attached to the indicated point of attachment through a ring member atom of a heterocycloalkyl ring. In some embodiments of monocyclic or polycyclic heterocycloalkyls, the monocyle or polycycle is attached to the indicated point of attachment through a ring member heteroatom of a heterocycloalkyl ring. In some embodiments of polycyclic heterocycloalkyls, the polycycle is attached to the indicated point of attachment through a ring member atom of a ring that is not a heterocycloalkyl ring, for example, an aryl ring, heteroaryl ring or a cycloalkyl ring. “Heterocycloalkyl” and “heterocycle” include sulfones, sulfoxides and N-oxides of tertiary nitrogen ring member atoms. Non-limiting examples of heterocycle groups include aziridinyl, azetidinyl, 1,3-dioxanyl, 1,4-dioxanyl, 1,3-dioxolanyl, morpholinyl, piperazinyl, pyrrolidinyl, piperidinyl, thiomorpholinyl, pyranyl, dihydropyridinyl, tetrahydropyridinyl, carabazolyl, xanthenyl, 1,3-benzodioxolyl, dihydroisoquinolinyl, dihydrocinnolinyl, dihydrobenzodioxinyl, isoindolinyl, dihydroisoindolyl and dihydroindolyl, and the like. The heterocycle groups may be optionally substituted unless specifically prohibited. Non-limiting examples of heterocycloalkyl groups may be referred to as cycloalkyl group having one or more carbon atoms substituted with O, NRⁿ, S, SO, SO₂, where n denotes any positive integer.

The term “hydrazinyl” as used herein, alone or in combination, refers to two amino groups joined by a single bond, i.e., —HN—NH—.

The term “hydroxy,” as used herein, alone or in combination, refers to —OH.

The term “hydroxyalkyl,” as used herein, alone or in combination, refers to a hydroxy group attached to a parent molecular moiety through an alkyl group. The term “lower hydroxyalkyl,” as used herein, alone or in combination, refers to a hydroxy group attached to a parent molecular moiety through a lower alkyl group, where “lower alkyl” is as defined herein.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means a moiety containing from 1 to and including 6 carbon atoms. A “lower alkyl,” for example, refers to an alkyl containing 1 to 6 carbon atoms (C1-C6), 1 to 5 carbon atoms (C1-C5), 1 to 4 carbon atoms (C1-C4), 1 to 3 carbon atoms (C1-C3), or 1 to 2 carbon atoms (C1-C2) (for example, an alkyl containing 1, 2, 3, 4, 5 or 6 carbon atoms; a C1, C2, C3, C4, C5 or C6 alkyl).

The term “lower aryl,” as used herein, alone or in combination, means a C4-C6 aryl group, for example, a C5-C6 aryl group. A lower aryl group sometimes is a C4-C6 aryl ring group, or C5-C6 aryl ring group for example, including without limitation, phenyl. The term may also refer to a C8-C10 bicyclic ring aryl group, for example, including without limitation, napthyl. Lower aryl groups, including phenyl or napthyl, may be optionally substituted as provided.

The term “lower heteroaryl,” as used herein, alone or in combination, means a four-membered, five-membered, or six-membered heteroaryl group. A lower heteroaryl group sometimes is (1) a monocyclic heteroaryl ring comprising five or six ring member atoms, of which between one and four of the ring member atoms may be heteroatoms chosen from O, S, and N, or (2) a bicyclic heteroaryl ring, where each of the fused rings comprises five or six ring member atoms, comprising between them one to four heteroatoms chosen from O, S, and N. Lower heteroaryl groups may be optionally substituted as provided.

The term “lower cycloalkyl,” as used herein, alone or in combination, means a monocyclic cycloalkyl having between three and six ring member atoms. Non-limiting examples of lower cycloalkyl include cyclopropyl, cyclobutyl, cyclopentyl, and cyclohexyl. Lower cycloalkyl groups may be optionally substituted as provided.

The term “lower heterocycloalkyl,” as used herein, alone or in combination, means a monocyclic heterocycloalkyl having between three and six ring member atoms, of which between one and four may be heteroatoms chosen from O, S, and N. Non-limiting examples of lower heterocycloalkyls include pyrrolidinyl, imidazolidinyl, pyrazolidinyl, piperidinyl, piperazinyl, and morpholinyl. Lower heterocycloalkyl groups may be optionally substituted as provided.

The term “lower amino,” as used herein, alone or in combination, refers to —NRR′, where R and R′ are independently chosen from hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptoalkyl,” as used herein, refers to a mercaptan or mercaptyl group attached to a parent molecule through an alkyl group (-alkyl-SH), where R is defined herein. The term “lower mercaptoalkyl,” as used herein, refers to a mercaptan or mercaptyl group attached to a parent molecule through a lower alkyl group, (-lower alkyl-SR), where “lower alkyl” and R are defined herein.

The terms “mercaptyl” or “mercaptan” as used herein, alone or in combination, refers to an —SH group.

The term “menthol,” as used herein, refers to 2-isopropyl-5-methylcyclohexanol. Menthol contains 3 chiral carbons and the term “menthol” encompasses all stereoisomers of the molecule unless specifically stated otherwise herein. For example, isomers of menthol include the (−)-menthol isomer ((1R,2S,5R)-2-isopropyl-5-methylcyclohexanol), (+)-menthol isomer ((1S,2R,5S)-2-isopropyl-5-methylcyclohexanol), (−)-isomenthol isomer ((1R,2S,5S)-2-isopropyl-5-methylcyclohexanol), (+)-isomenthol isomer ((1S,2R,5R)-2-isopropyl-5-methylcyclohexanol), (−)-neomenthol isomer ((1R,2R,5S)-2-isopropyl-5-methylcyclohexanol), (+)-neomenthol isomer ((1S,2S,5R)-2-isopropyl-5-methylcyclohexanol), (−)-neoisomenthol isomer ((1S,2S,5S)-2-isopropyl-5-methylcyclohexanol) and (+)-neoisomenthol isomer ((1R,2R,5R)-2-isopropyl-5-methylcyclohexanol).

The term “menthyl,” as used herein, refers to a radical derived from menthol. Typically, a menthyl radical can be linked to another chemical group through the oxygen atom of the menthyl group.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “partially unsaturated,” as used herein, alone or in combination, refers to a straight-chain, branched-chain or ring moiety that includes at least one double or triple bond and that is not fully saturated. The term “partially unsaturated” when used in reference to a ring moiety means a ring having one or multiple sites of unsaturation but does not include aryl rings or heteroaryl rings as defined herein.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “piperitol,” as used herein, refers to p-menth-1-en-3-ol. Piperitol contains 2 chiral carbons and the term “piperitol” encompasses all stereoisomers of the molecule unless specifically stated otherwise herein. For example, isomers of piperitol include (3R,4R)-piperitol (also referred to as trans-piperitol) and (3S,4R)-piperitol (also referred to as cis-piperitol).

The term “ring member atoms,” as used herein, refers to all of the atoms that form the covalent structure of a cyclic ring structure.

By “saturated” is meant that the carbon-containing group contains no carbon-carbon double or triple bonds.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a R^{s}(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether where the oxygen is replaced with sulfur. The oxidized derivatives of the thio group, namely sulfinyl and sulfonyl, are included in the definition of thia and thio.

The terms “thiol” and “mercapto”, as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Non-limiting examples include trimethylsilyl, tert-butyldimethylsilyl, triphenylsilyl and the like.

The term “ureido,” as used herein, alone or in combination, refers to the univalent radical NH₂CONH— derived from urea. Non-limiting examples include ureidoproprionate and ureidosuccinate.

Non-limiting examples of compounds are provided in the following Table A.

TABLE A

| Compound | | |
|---|---|---|
| Designation | IUPAC Name | Structure |
| Cmpd10 | N1-(5-((8-(4-fluoro-2- isopropoxyphenyl)quinazolin-2- yl)amino)-2-methylphenyl)-N4- methylterephthalamide | 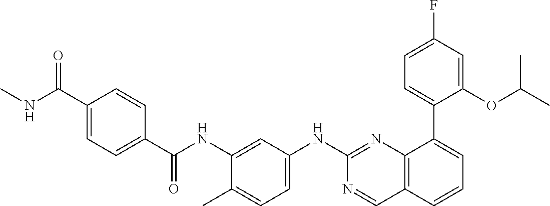 [SMILES: CNC(=O)c1ccc(C(=O)Nc2cc(Nc3ncc4cccc(-c5ccc(F)cc5OC(C)C)c4n3)ccc2C)cc1] |
| Cmpd11 | N1-(5-((8-(4-fluoro-2- isopropoxyphenyl)quinazolin-2- yl)amino)-2-methylphenyl)-N4,N4- dimethylterephthalamide | 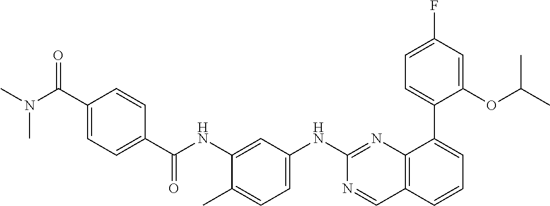 [SMILES: Cc1ccc(Nc2ncc3cccc(-c4ccc(F)cc4OC(C)C)c3n2)cc1NC(=O)c1ccc(C(=O)N(C)C)cc1] |
| Cmpd12 | N-(5-((8-(4-fluoro-2- isopropoxyphenyl)quinazolin-2- yl)amino)-2- methylphenyl)terephthalamide | 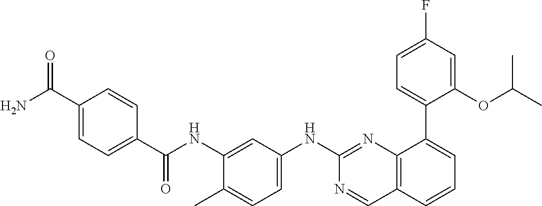 [SMILES: Cc1ccc(Nc2ncc3cccc(-c4ccc(F)cc4OC(C)C)c3n2)cc1NC(=O)c1ccc(C(N)=O)cc1] |
| Cmpd13 | ethyl 4-((5-((8-bromoquinazolin-2- yl)amino)-2- methylphenyl)carbamoyl)benzoate | 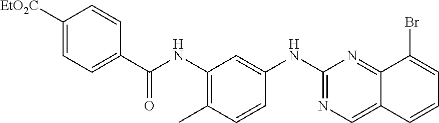 [SMILES: CCOC(=O)c1ccc(C(=O)Nc2cc(Nc3ncc4cccc(Br)c4n3)ccc2C)cc1] |
| Cmpd14 | ethyl 4-((3-((8-bromoquinazolin-2- yl)amino)-5- methylphenyl)carbamoyl)benzoate | 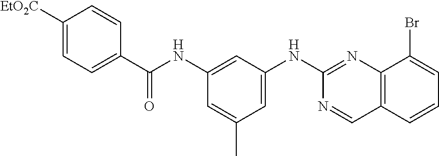 [SMILES: CCOC(=O)c1ccc(C(=O)Nc2cc(C)cc(Nc3ncc4cccc(Br)c4n3)c2)cc1] |
| Cmpd15 | ethyl 4-((5-((8-bromoquinazolin-2- yl)amino)-2- methoxyphenyl)carbamoyl)benzoate | 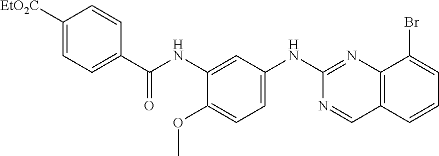 [SMILES: CCOC(=O)c1ccc(C(=O)Nc2cc(Nc3ncc4cccc(Br)c4n3)ccc2OC)cc1] |
| Cmpd16 | ethyl 4-((3-((8-bromoquinazolin-2- yl)amino)phenyl)carbamoyl)benzoate | 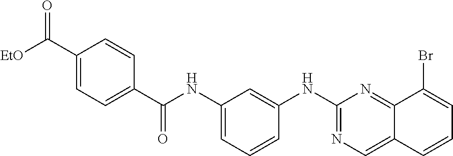 [SMILES: CCOC(=O)c1ccc(C(=O)Nc2cccc(Nc3ncc4cccc(Br)c4n3)c2)cc1] |
| Cmpd17 | N1-[5-[(8-bromoquinazolin-2- yl)amino]-2-methyl-phenyl]-N4- methyl-terephthalamide | 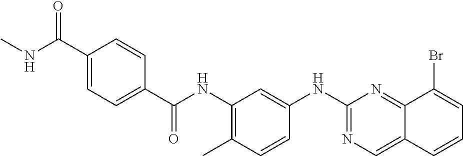 [SMILES: CNC(=O)c1ccc(C(=O)Nc2cc(Nc3ncc4cccc(Br)c4n3)ccc2C)cc1] |
| Cmpd18 | N4-[5-[(8-bromoquinazolin-2- yl)amino]-2-methyl-phenyl]-N1,N1- dimethyl-terephthalamide | 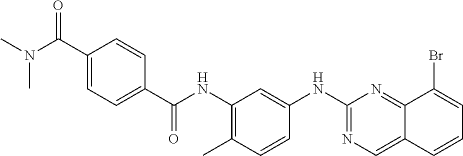 [SMILES: Cc1ccc(Nc2ncc3cccc(Br)c3n2)cc1NC(=O)c1ccc(C(=O)N(C)C)cc1] |
| Cmpd19 | ethyl 4-((5-((8-bromoquinazolin-2- yl)amino)-2- methylphenyl)carbamoyl)benzoate | 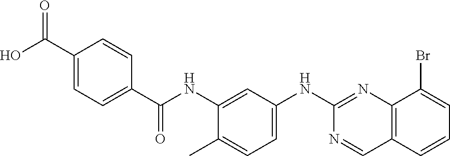 [SMILES: Cc1ccc(Nc2ncc3cccc(Br)c3n2)cc1NC(=O)c1ccc(C(=O)O)cc1] |
| Cmpd20 | 4-((4-((5-bromoquinazolin-2- yl)amino)-2- methylphenyl)carbamoyl)benzoic acid | 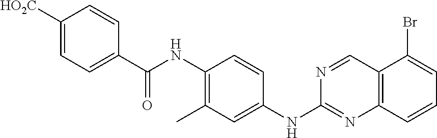 [SMILES: Cc1cc(Nc2ncc3c(Br)cccc3n2)ccc1NC(=O)c1ccc(C(=O)O)cc1] |
| Cmpd21 | 4-((2-methyl-5-(quinazolin-2- ylamino)phenyl)carbamoyl)benzoic acid | 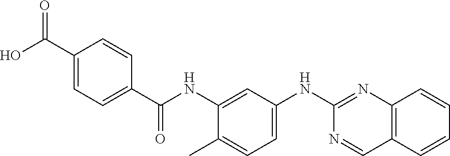 [SMILES: Cc1ccc(Nc2ncc3ccccc3n2)cc1NC(=O)c1ccc(C(=O)O)cc1] |
| Cmpd24 | 4-((5-((7-(4-fluoro-2- isopropoxyphenyl)quinazolin-2- yl)amino)-2- methylphenyl)carbamoyl)benzoic acid | 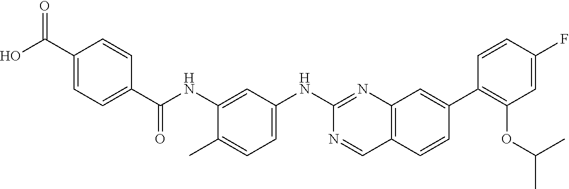 [SMILES: Cc1ccc(Nc2ncc3ccc(-c4ccc(F)cc4OC(C)C)cc3n2)cc1NC(=O)c1ccc(C(=O)O)cc1] |
| Cmpd25 | 4-((5-((8-((2- isopropoxyphenyl)ethynyl)quinazolin- 2-yl)amino)-2- methylphenyl)carbamoyl)benzoic acid | 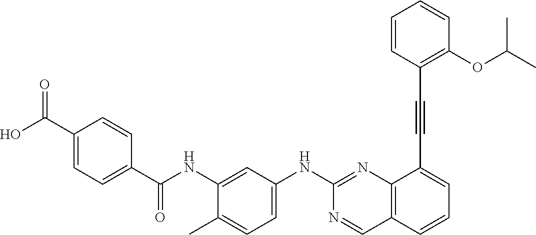 [SMILES: Cc1ccc(Nc2ncc3cccc(C#Cc4ccccc4OC(C)C)c3n2)cc1NC(=O)c1ccc(C(=O)O)cc1] |
| Cmpd26 | 4-((4-((8-(4-fluoro-2- isopropoxyphenyl)quinazolin-2- yl)amino)-2- methylphenyl)carbamoyl)benzoic acid | 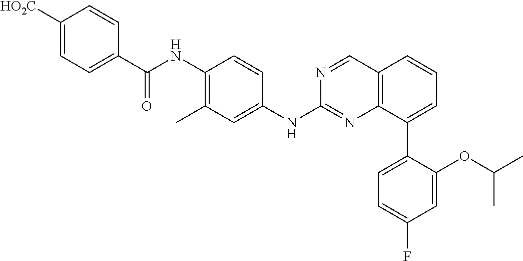 [SMILES: Cc1cc(Nc2ncc3cccc(-c4ccc(F)cc4OC(C)C)c3n2)ccc1NC(=O)c1ccc(C(=O)O)cc1] |
| Cmpd27 | 4-((4-((5-(4-fluoro-2- isopropoxyphenyl)quinazolin-2- yl)amino)-2- methylphenyl)carbamoyl)benzoic acid | 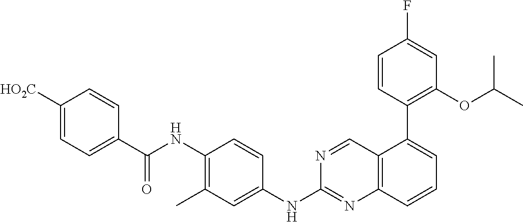 [SMILES: Cc1cc(Nc2ncc3c(-c4ccc(F)cc4OC(C)C)cccc3n2)ccc1NC(=O)c1ccc(C(=O)O)cc1] |
| Cmpd28 | 4-(5-((8-(4-fluoro-2- isopropoxyphenyl)quinazolin-2- yl)amino)-2- methylbenzamido)benzoic acid | 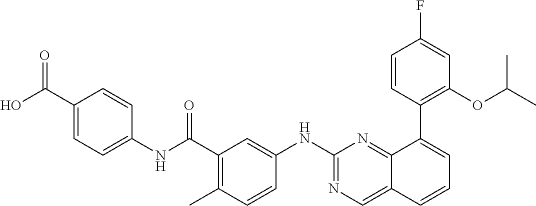 [SMILES: Cc1ccc(Nc2ncc3cccc(-c4ccc(F)cc4OC(C)C)c3n2)cc1C(=O)Nc1ccc(C(=O)O)cc1] |
| Cmpd29 | 4-((5-((5-(2-((2- hydroxyethyl)amino)pyrimidin-5- yl)quinazolin-2-yl)amino)-2- methylphenyl)carbamoyl)benzoic acid | 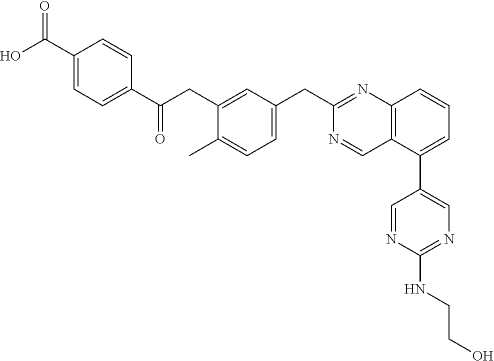 [SMILES: Cc1ccc(Cc2ncc3c(-c4cnc(NCCO)nc4)cccc3n2)cc1CC(=O)c1ccc(C(=O)O)cc1] |
| Cmpd30 | 4-((5-((5-(2-chloropyridin-4- yl)quinazolin-2-yl)amino)-2- methylphenyl)carbamoyl)benzoic acid | 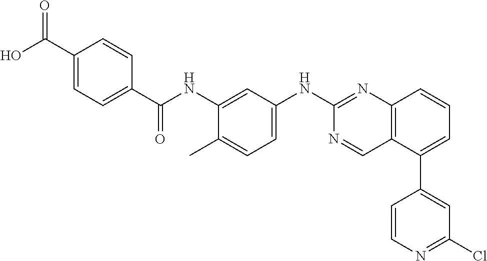 [SMILES: Cc1ccc(Nc2ncc3c(-c4ccnc(Cl)c4)cccc3n2)cc1NC(=O)c1ccc(C(=O)O)cc1] |
| Cmpd31 | 4-((5-((5-mesitylquinazolin-2- yl)amino)-2- methylphenyl)carbamoyl)benzoic acid | 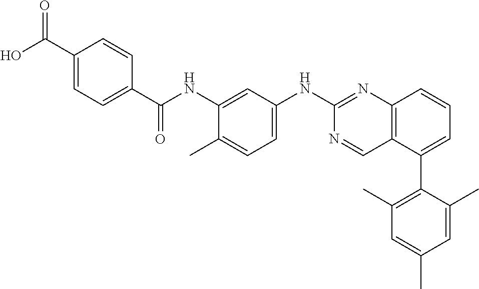 [SMILES: Cc1cc(C)c(-c2cccc3nc(Nc4ccc(C)c(NC(=O)c5ccc(C(=O)O)cc5)c4)ncc23)c(C)c1] |
| Cmpd32 | 4-((2-methyl-5-((6-(2-(pyrrolidin-1- yl)pyridin-3-yl)quinazolin-2- yl)amino)phenyl)carbamoyl)benzoic acid | 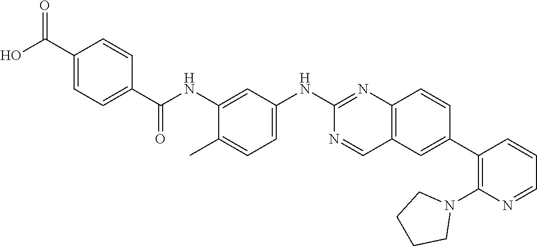 [SMILES: Cc1ccc(Nc2ncc3cc(-c4cccnc4N4CCCC4)ccc3n2)cc1NC(=O)c1ccc(C(=O)O)cc1] |
| Cmpd33 | 4-((5-((6-(1H-indol-2-yl)quinazolin-2- yl)amino)-2- methylphenyl)carbamoyl)benzoic acid | 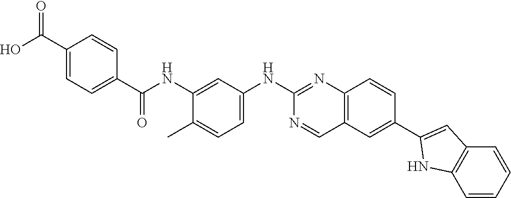 [SMILES: Cc1ccc(Nc2ncc3cc(-c4cc5ccccc5[nH]4)ccc3n2)cc1NC(=O)c1ccc(C(=O)O)cc1] |
| Cmpd34 | 4-((5-((7-(benzo[d]oxazol-5- yl)quinazolin-2-yl)amino)-2- methylphenyl)carbamoyl)benzoic acid | 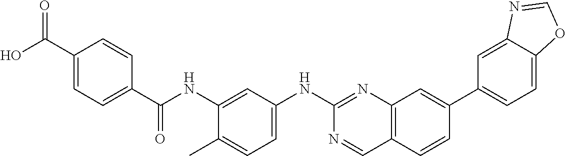 [SMILES: Cc1ccc(Nc2ncc3ccc(-c4ccc5ocnc5c4)cc3n2)cc1NC(=O)c1ccc(C(=O)O)cc1] |
| Cmpd36 | 4-((5-((7-(1H-pyrazol-3-yl)quinazolin- 2-yl)amino)-2- methylphenyl)carbamoyl)benzoic acid | 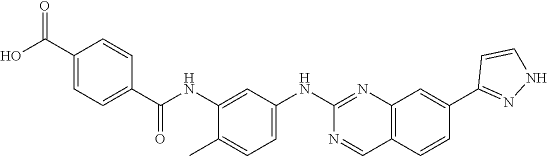 [SMILES: Cc1ccc(Nc2ncc3ccc(-c4cc[nH]n4)cc3n2)cc1NC(=O)c1ccc(C(=O)O)cc1] |
| Cmpd37 | 4-((5-((8-(2,4-dimethoxypyrimidin-5- yl)quinazolin-2-yl)amino)-2- methylphenyl)carbamoyl)benzoic acid | 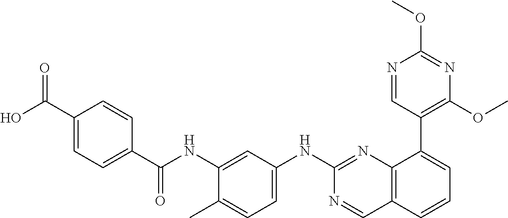 [SMILES: COc1ncc(-c2cccc3cnc(Nc4ccc(C)c(NC(=O)c5ccc(C(=O)O)cc5)c4)nc23)c(OC)n1] |
| Cmpd38 | 4-((5-((8-(imidazo[1,2-a]pyridin-6- yl)quinazolin-2-yl)amino)-2- methylphenyl)carbamoyl)benzoic acid | 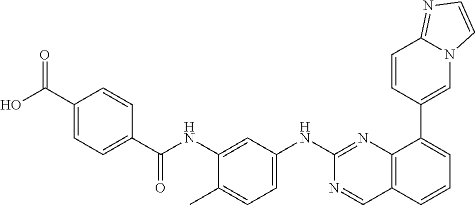 [SMILES: Cc1ccc(Nc2ncc3cccc(-c4ccc5nccn5c4)c3n2)cc1NC(=O)c1ccc(C(=O)O)cc1] |
| Cmpd39 | 4-((5-((8-(6-hydroxypyridin-3- yl)quinazolin-2-yl)amino)-2- methylphenyl)carbamoyl)benzoic acid | 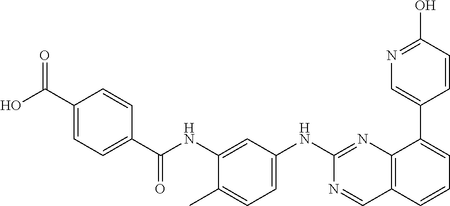 [SMILES: Cc1ccc(Nc2ncc3cccc(-c4ccc(O)nc4)c3n2)cc1NC(=O)c1ccc(C(=O)O)cc1] |
| Cmpd40 | 4-((5-((7-(1H-indol-5-yl)quinazolin-2- yl)amino)-2- methylphenyl)carbamoyl)benzoic acid | 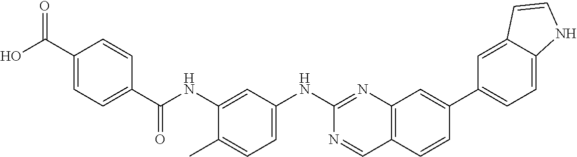 [SMILES: Cc1ccc(Nc2ncc3ccc(-c4ccc5[nH]ccc5c4)cc3n2)cc1NC(=O)c1ccc(C(=O)O)cc1] |
| Cmpd42 | 4-((5-((6-(4-(benzyloxy)-2- formylphenyl)quinazolin-2-yl)amino)- 2-methylphenyl)carbamoyl)benzoic acid | 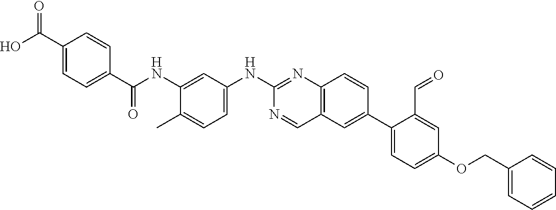 [SMILES: Cc1ccc(Nc2ncc3cc(-c4ccc(OCc5ccccc5)cc4C=O)ccc3n2)cc1NC(=O)c1ccc(C(=O)O)cc1] |
| Cmpd43 | 3-(2-((3-(4-carboxybenzamido)-4- methylphenyl)amino)quinazolin-5-yl)- 5-nitrobenzoic acid | 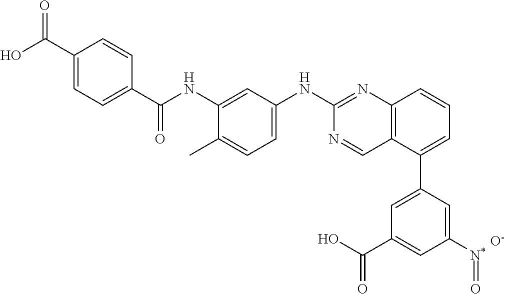 |
| Cmpd44 | N-(5-((8-(4-fluoro-2- isopropoxyphenyl)quinazolin-2- yl)amino)-2-methylphenyl)-4-(4- methylpiperazine-1- carbonyl)benzamide | 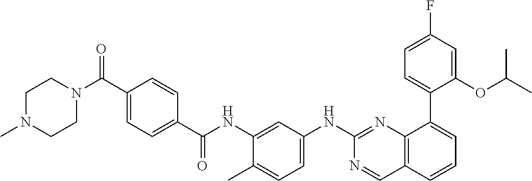 [SMILES: Cc1ccc(Nc2ncc3cccc(-c4ccc(F)cc4OC(C)C)c3n2)cc1NC(=O)c1ccc(C(=O)N2CCN(C)CC2)cc1] |
| Cmpd45 | N-(5-((8-(4-fluoro-2- isopropoxyphenyl)quinazolin-2- yl)amino)-2-methylphenyl)-4- (morpholine-4-carbonyl)benzamide | 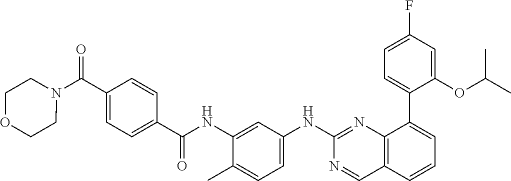 [SMILES: Cc1ccc(Nc2ncc3cccc(-c4ccc(F)cc4OC(C)C)c3n2)cc1NC(=O)c1ccc(C(=O)N2CCOCC2)cc1] |
| Cmpd46 | N1-(5-((5-(1-isobutyl-1H-pyrazol-5- yl)quinazolin-2-yl)amino)-2- methylphenyl)-N4- methylterephthalamide | 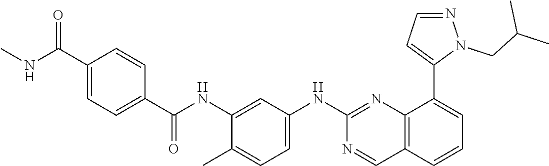 [SMILES: CNC(=O)c1ccc(C(=O)Nc2cc(Nc3ncc4cccc(-c5ccnn5CC(C)C)c4n3)ccc2C)cc1] |
| Cmpd47 | N1-(5-((8-(1-isobutyl-1H-pyrazol-5- yl)quinazolin-2-yl)amino)-2- methylphenyl)-N4,N4- dimethylterephthalamide | 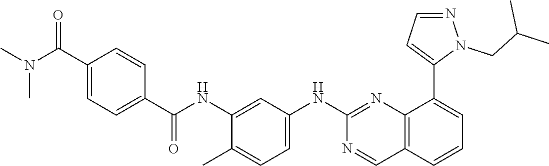 [SMILES: Cc1ccc(Nc2ncc3cccc(-c4ccnn4CC(C)C)c3n2)cc1NC(=O)c1ccc(C(=O)N(C)C)cc1] |
| Cmpd48 | N-(5-((8-(1-isobutyl-1H-pyrazol-5- yl)quinazolin-2-yl)amino)-2- methylphenyl)-4-(4- methylpiperazine-1- carbonyl)benzamide | 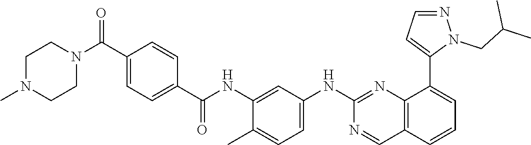 [SMILES: Cc1ccc(Nc2ncc3cccc(-c4ccnn4CC(C)C)c3n2)cc1NC(=O)c1ccc(C(=O)N2CCN(C)CC2)cc1] |
| Cmpd49 | N-(5-((8-(4-fluoro-2- isopropoxyphenyl)quinazolin-2- yl)amino)-2-methylphenyl)acetamide | 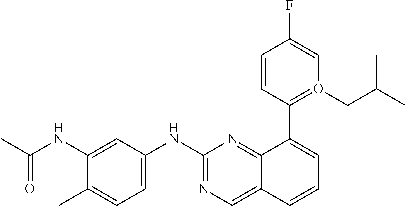 |
| Cmpd50 | N-(5-((8-(4-fluoro-2- isopropoxyphenyl)quinazolin-2- yl)amino)-2- methoxyphenyl)acetamide | 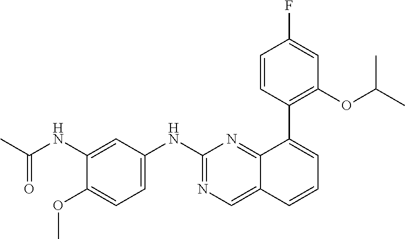 [SMILES: COc1ccc(Nc2ncc3cccc(-c4ccc(F)cc4OC(C)C)c3n2)cc1NC(C)=O] |
| Cmpd51 | N1-(8-(4-fluoro-2- isopropoxyphenyl)quinazolin-2-yl)-4- methylbenzene-1,3-diamine | 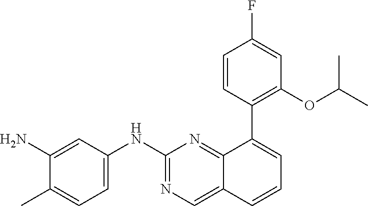 [SMILES: Cc1ccc(Nc2ncc3cccc(-c4ccc(F)cc4OC(C)C)c3n2)cc1N] |
| Cmpd52 | 2-(dimethylamino)-N-(5-((8-(4-fluoro- 2-isopropoxyphenyl)quinazolin-2- yl)amino)-2-methylphenyl)acetamide | 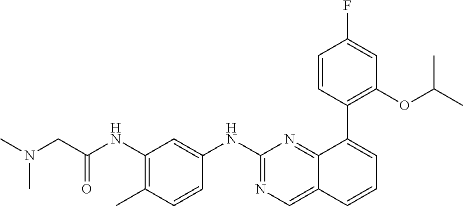 [SMILES: Cc1ccc(Nc2ncc3cccc(-c4ccc(F)cc4OC(C)C)c3n2)cc1NC(=O)CN(C)C] |
| Cmpd53 | N-(5-((8-(4-fluoro-2- isopropoxyphenyl)quinazolin-2- yl)amino)-2-methylphenyl)acrylamide | 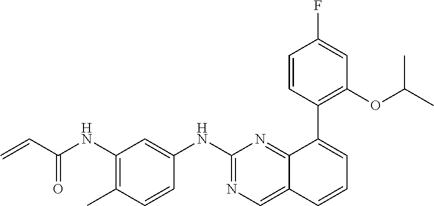 [SMILES: C=CC(=O)Nc1cc(Nc2ncc3cccc(-c4ccc(F)cc4OC(C)C)c3n2)ccc1C] |
| Cmpd54 | N-(5-((8-(4-fluoro-2- isopropoxyphenyl)quinazolin-2- yl)amino)-2- methoxyphenyl)acrylamide | 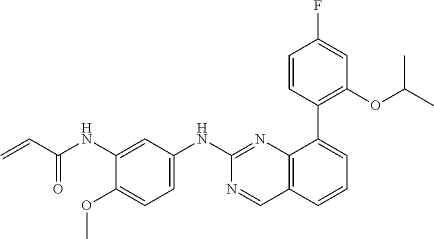 [SMILES: C=CC(=O)Nc1cc(Nc2ncc3cccc(-c4ccc(F)cc4OC(C)C)c3n2)ccc1OC] |
| Cmpd55 | N-(5-((8-(4-fluoro-2- isopropoxyphenyl)quinazolin-2- yl)amino)-2-methylphenyl)-2- (methylamino)acetamide | 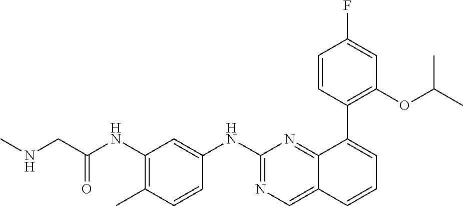 [SMILES: CNCC(=O)Nc1cc(Nc2ncc3cccc(-c4ccc(F)cc4OC(C)C)c3n2)ccc1C] |
| Cmpd57 | (5-((8-(4-fluoro-2- isopropoxyphenyl)quinazolin-2- yl)amino)-2-methylphenyl)(4- methylpiperazin-1-yl)methanone | 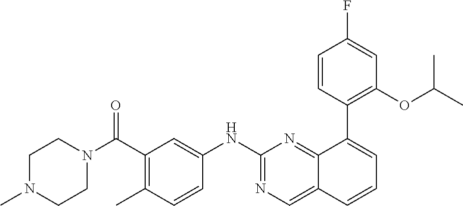 [SMILES: Cc1ccc(Nc2ncc3cccc(-c4ccc(F)cc4OC(C)C)c3n2)cc1C(=O)N1CCN(C)CC1] |
| Cmpd58 | N-(5-((8-(4-fluoro-2- isopropoxyphenyl)quinazolin-2- yl)amino)-2-methylphenyl)-2-(4- methylpiperazin-1-yl)acetamide | 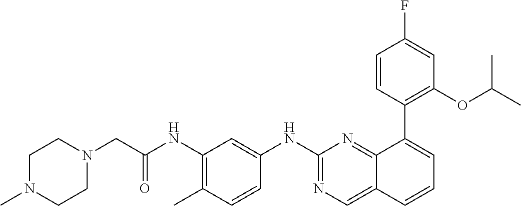 [SMILES: Cc1ccc(Nc2ncc3cccc(-c4ccc(F)cc4OC(C)C)c3n2)cc1NC(=O)CN1CCN(C)CC1] |
| Cmpd59 | N-(5-((8-(4-fluoro-2- isopropoxyphenyl)quinazolin-2- yl)amino)-2-methylphenyl)-4- methylpiperazine-1-carboxamide | 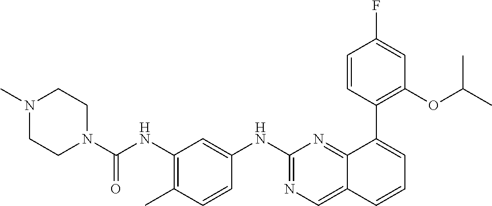 [SMILES: Cc1ccc(Nc2ncc3cccc(-c4ccc(F)cc4OC(C)C)c3n2)cc1NC(=O)N1CCN(C)CC1] |
| Cmpd60 | N-(5-((8-(4-fluoro-2- isopropoxyphenyl)quinazolin-2- yl)amino)-2- methylphenyl)nicotinamide | 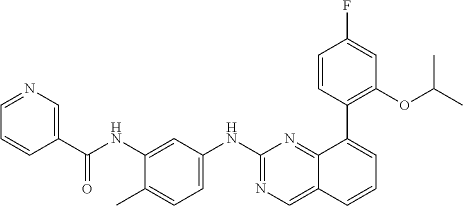 [SMILES: Cc1ccc(Nc2ncc3cccc(-c4ccc(F)cc4OC(C)C)c3n2)cc1NC(=O)c1cccnc1] |
| Cmpd61 | N-(5-((8-(4-fluoro-2- isopropoxyphenyl)quinazolin-2- yl)amino)-2-methylphenyl)-5- (trifluoromethyl)nicotinamide | 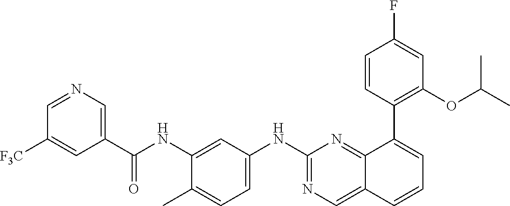 [SMILES: Cc1ccc(Nc2ncc3cccc(-c4ccc(F)cc4OC(C)C)c3n2)cc1NC(=O)c1cncc(C(F)(F)F)c1] |
| Cmpd62 | N-(5-((8-(4-fluoro-2- isopropoxyphenyl)quinazolin-2- yl)amino)-2-methylphenyl)pyrimidine- 4-carboxamide | 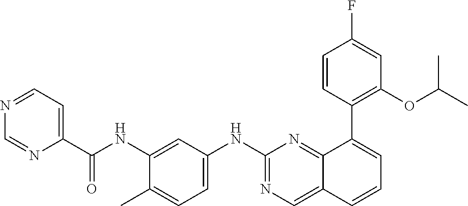 [SMILES: Cc1ccc(Nc2ncc3cccc(-c4ccc(F)cc4OC(C)C)c3n2)cc1NC(=O)c1ccncn1] |
| Cmpd63 | N-(5-((8-(4-fluoro-2- isopropoxyphenyl)quinazolin-2- yl)amino)-2-methylphenyl)oxazole-2- carboxamide | 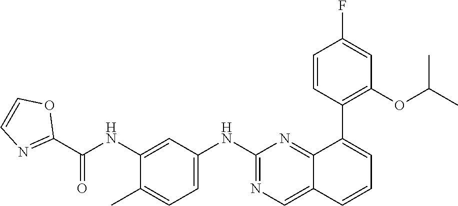 [SMILES: Cc1ccc(Nc2ncc3cccc(-c4ccc(F)cc4OC(C)C)c3n2)cc1NC(=O)c1ncco1] |
| Cmpd65 | N-(5-((8-(4-fluoro-2- isopropoxyphenyl)quinazolin-2- yl)amino)-2-methylphenyl)-1-methyl- 1H-pyrazole-3-carboxamide | 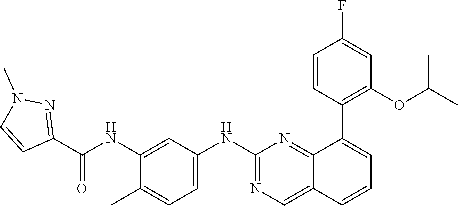 [SMILES: Cc1ccc(Nc2ncc3cccc(-c4ccc(F)cc4OC(C)C)c3n2)cc1NC(=O)c1ccn(C)n1] |
| Cmpd66 | N-(5-((8-(4-fluoro-2- isopropoxyphenyl)quinazolin-2- yl)amino)-2-methylphenyl)-1- methylpiperidine-4-carboxamide | 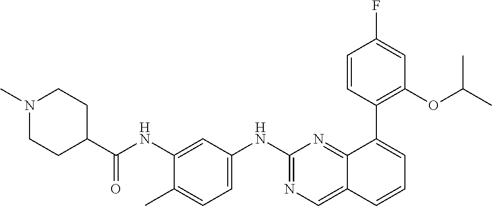 [SMILES: Cc1ccc(Nc2ncc3cccc(-c4ccc(F)cc4OC(C)C)c3n2)cc1NC(=O)C1CCN(C)CC1] |
| Cmpd67 | N-(5-((8-(4-fluoro-2- isopropoxyphenyl)quinazolin-2- yl)amino)-2-methoxyphenyl)-1- methylpiperidine-4-carboxamide | 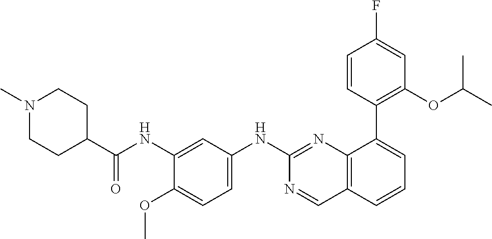 [SMILES: COc1ccc(Nc2ncc3cccc(-c4ccc(F)cc4OC(C)C)c3n2)cc1NC(=O)C1CCN(C)CC1] |
| Cmpd68 | (S)-N-(5-((8-(4-fluoro-2- isopropoxyphenyl)quinazolin-2- yl)amino)-2-methylphenyl)-1- methylpyrrolidine-2-carboxamide | 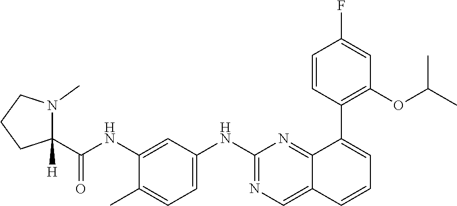 [SMILES: Cc1ccc(Nc2ncc3cccc(-c4ccc(F)cc4OC(C)C)c3n2)cc1NC(=O)[C@@H]1CCCN1C] |
| Cmpd69 | N-(5-((8-(4-fluoro-2- isopropoxyphenyl)quinazolin-2- yl)amino)-2-methylphenyl)-1- methylazetidine-3-carboxamide | 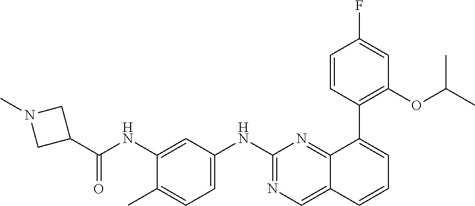 [SMILES: Cc1ccc(Nc2ncc3cccc(-c4ccc(F)cc4OC(C)C)c3n2)cc1NC(=O)C1CN(C)C1] |
| Cmpd70 | N-(5-((8-(4-fluoro-2- isopropoxyphenyl)quinazolin-2- yl)amino)-2-methylphenyl)tetrahydro- 2H-pyran-4-carboxamide | 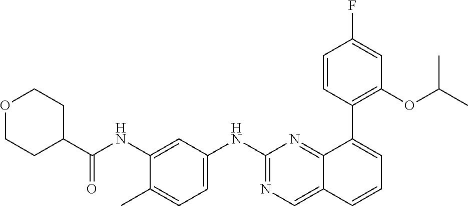 [SMILES: Cc1ccc(Nc2ncc3cccc(-c4ccc(F)cc4OC(C)C)c3n2)cc1NC(=O)C1CCOCC1] |
| Cmpd71 | (R)-N-(5-((8-(4-fluoro-2- isopropoxyphenyl)quinazolin-2- yl)amino)-2-methylphenyl)-1- methylpyrrolidine-2-carboxamide | 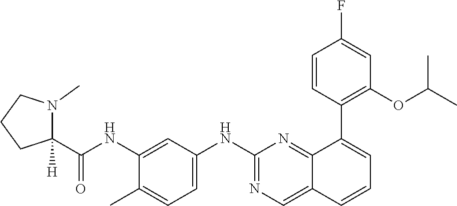 [SMILES: Cc1ccc(Nc2ncc3cccc(-c4ccc(F)cc4OC(C)C)c3n2)cc1NC(=O)[C@H]1CCCN1C] |
| Cmpd72 | N-(5-((8-(4-fluoro-2- isopropoxyphenyl)quinazolin-2- yl)amino)-2-methylphenyl)-1H- pyrazole-3-carboxamide | 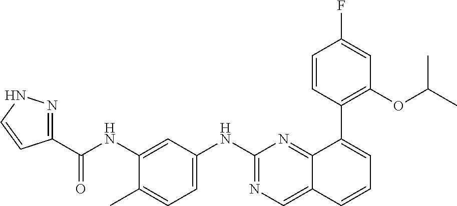 [SMILES: Cc1ccc(Nc2ncc3cccc(-c4ccc(F)cc4OC(C)C)c3n2)cc1NC(=O)c1cc[nH]n1] |
| Cmpd73 | N-(5-((8-(4-fluoro-2- isopropoxyphenyl)quinazolin-2- yl)amino)-2-methylphenyl)-1H- imidazole-2-carboxamide | 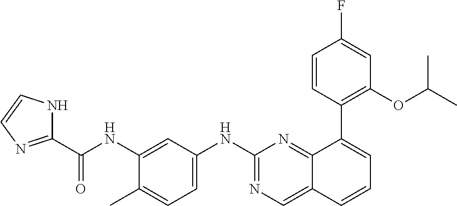 [SMILES: Cc1ccc(Nc2ncc3cccc(-c4ccc(F)cc4OC(C)C)c3n2)cc1NC(=O)c1ncc[nH]1] |
| Cmpd74 | N-(5-((8-(4-fluoro-2- isopropoxyphenyl)quinazolin-2- yl)amino)-2-methylphenyl)oxazole-4- carboxamide | 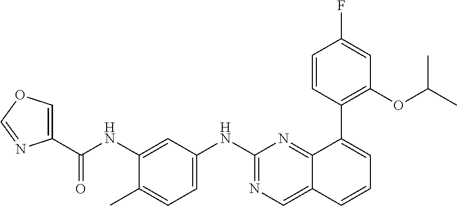 [SMILES: Cc1ccc(Nc2ncc3cccc(-c4ccc(F)cc4OC(C)C)c3n2)cc1NC(=O)c1cocn1] |
| Cmpd76 | N-(4-((8-(4-fluoro-2- isopropoxyphenyl)quinazolin-2- yl)amino)-2-methylphenyl)-4-(4- methylpiperazine-1- carbonyl)benzamide | 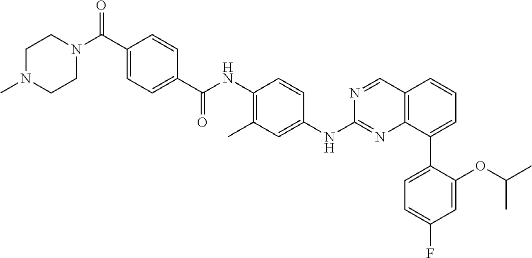 [SMILES: Cc1cc(Nc2ncc3cccc(-c4ccc(F)cc4OC(C)C)c3n2)ccc1NC(=O)c1ccc(C(=O)N2CCN(C)CC2)cc1] |
| Cmpd77 | N1-(8-((2- isopropoxyphenyl)ethynyl)quinazolin- 2-yl)-4-methylbenzene-1,3-diamine | 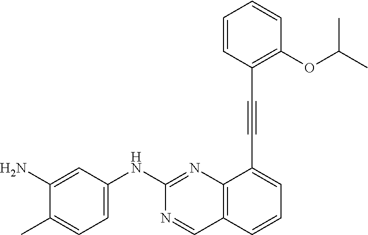 [SMILES: Cc1ccc(Nc2ncc3cccc(C#Cc4ccccc4OC(C)C)c3n2)cc1N] |
| Cmpd78 | N-(5-((8-((2- isopropoxyphenyl)ethynyl)quinazolin- 2-yl)amino)-2-methylphenyl)-1- methylpiperidine-4-carboxamide | 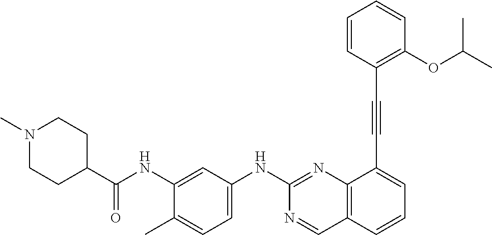 [SMILES: Cc1ccc(Nc2ncc3cccc(C#Cc4ccccc4OC(C)C)c3n2)cc1NC(=O)C1CCN(C)CC1] |
| Cmpd79 | N-(5-((8-((2- isopropoxyphenyl)ethynyl)quinazolin- 2-yl)amino)-2- methylphenyl)acrylamide | 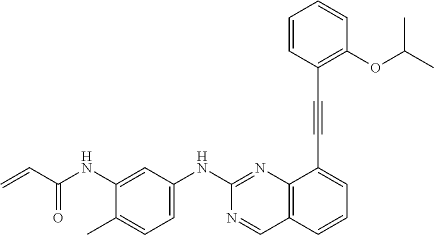 [SMILES: C=CC(=O)Nc1cc(Nc2ncc3cccc(C#Cc4ccccc4OC(C)C)c3n2)ccc1C] |
| Cmpd80 | N-(5-((8-(4-fluoro-2- isopropoxyphenyl)quinazolin-2- yl)amino)-2- methylphenyl)propiolamide | 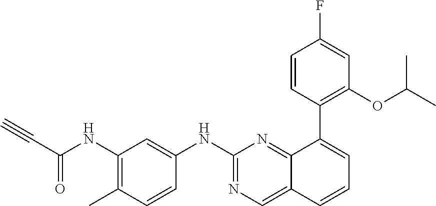 [SMILES: C#CC(=O)Nc1cc(Nc2ncc3cccc(-c4ccc(F)cc4OC(C)C)c3n2)ccc1C] |
| Cmpd81 | N-(5-((8-(4-fluoro-2- isopropoxyphenyl)quinazolin-2- yl)amino)-2-methylphenyl)-4-((4- methylpiperazin-1-yl)methyl)-3- (trifluoromethyl)benzamide | 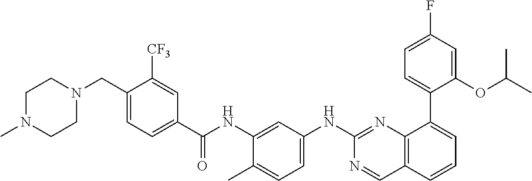 [SMILES: Cc1ccc(Nc2ncc3cccc(-c4ccc(F)cc4OC(C)C)c3n2)cc1NC(=O)c1ccc(CN2CCN(C)CC2)c(C(F)(F)F)c1] |
| Cmpd82 | N1-[8-(4-fluoro-2-isopropoxy- phenyl)quinazolin-2-yl]-4-methoxy- benzene-1,3-diamine | 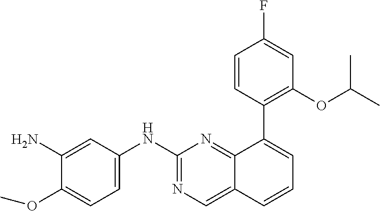 [SMILES: COc1ccc(Nc2ncc3cccc(-c4ccc(F)cc4OC(C)C)c3n2)cc1N] |

Compounds in Table A may be provided as a pharmaceutically acceptable salt, such as a hydrochloride salt for example. In Table A, the following compound designations are not included and are held in reserve: Cmpd22, Cmpd23, Cmpd35, Cmpd41, Cmpd56, Cmpd64 and Cmpd75.

### Compositions

A composition can contain a compound herein. A compound in a composition can inhibit an activity of a protein kinase (PK; for example, a PK polypeptide). A PK activity can include a PK binding activity (for example, binding of a PK to a substrate that the PK phosphorylates and/or binding of a PK to a binding partner polypeptide that the PK does not phosphorylate) and/or PK catalytic activity (for example, PK substrate phosphorylation activity). In certain instances a compound herein is capable of effectively inhibiting, moderately inhibiting and/or selectively inhibiting a PK activity. A composition containing a compound herein can be for inhibition of a PK activity, inhibition of an activity of two or more PKs, preparation of a medicament and/or for preparation of a treatment of a PK-associated condition.

In certain embodiments, a compound in a composition is or has been isolated (for example, isolated from other types of molecules). A compound sometimes is at least about 80% pure, by weight, in a composition and sometime is at least about 85% pure, at least about 90% pure, at least about 95% pure, at least about 99% pure, or at least about 99.5% pure. A compound n that is x % pure, by weight, in a composition includes x % by weight of the compound and y % of components other than the compound, where y %=100%−x %, by weight. For example, a compound that is 90% pure in a composition contains 90% by weight of the compound and 10% by weight of components other than the compound.

A composition containing a compound herein can be a pharmaceutical composition. A pharmaceutical composition can include a compound herein as an active ingredient and one or more pharmaceutically acceptable additives, including one or more pharmaceutically acceptable excipients. One or more pharmaceutically acceptable excipients in a pharmaceutical composition typically form a carrier for the active ingredient. Non-limiting examples of excipient additives include a pharmaceutically acceptable solvent, diluent, isotonic agent, buffering agent, stabilizer, preservative, antioxidant, vasoconstrictive agent, antibacterial agent, antifungal agent, adsorption delaying agent, sustained release agent (for example, for example, U.S. Pat. No. 5,624,677), and the like. One or more additives can be combined with an active ingredient for the manufacture of a pharmaceutical composition by a method known in the art. A pharmaceutical composition sometimes is prepared as a solid (for example, powder) or liquid (for example, aqueous solution, emulsion (for example, micro-emulsion, nano-emulsion)).

Non-limiting examples of solvents and diluents include water, saline, dextrose, ethanol, glycerol, oil, water-miscible organic cosolvents such as acetone or dimethyl sulfoxide (DMSO), and the like. Non-limiting examples of isotonic agents include sodium chloride, dextrose, mannitol, glucose, sucrose, sorbitol, lactose, and the like. Non-limiting examples of buffering agents include bicarbonate, phosphate, and the like. Phosphate-buffered saline (PBS), which may be buffered to provide a neutral pH, or in certain embodiments an acidic pH, sometimes is utilized. Non-limiting examples of stabilizers include gelatin, albumin, and the like. Non-limiting examples of a preservatives include gentamicin, merthiolate, chlorocresol and the like.

Water or saline, when used for preparing a pharmaceutical composition, may be buffered or not buffered. Non-limiting examples of saline solutions that can be used to prepare a pharmaceutical composition include lactated Ringer's solution, acetated Ringer's solution, intravenous sugar solutions (for example, 5% dextrose in normal saline (D5NS), 10% dextrose in normal saline (D10NS), 5% dextrose in half-normal saline (D5HNS) and 10% dextrose in half-normal saline (D10HNS)). Non-limiting example of buffered saline solutions and related solutions include phosphate buffered saline (PBS), TRIS-buffered saline (TBS), Hank's balanced salt solution (HBSS), Earle's balanced salt solution (EBSS), standard saline citrate (SSC), HEPES-buffered saline (HBS), and Gey's balanced salt solution (GBSS).

In certain implementations, an additive enhances solubility and/or bioavailability of an active ingredient. A solubility-enhancing additive may include one or more of: a lipid, polyethylene glycol (PEG), polysorbate, glycerol, glycerin, dimethylacetamide, triacetin, an oil (for example, a vegetable oil), or combination thereof. In certain instances, solubility of an active ingredient is characterized by a particular amount of excipient that confers solubility to the active ingredient.

Any suitable antioxidant can be included in a pharmaceutical composition, non-limiting examples of which include (1) butylated hydroxytoluene (BHT), (2) butylated hydroxyanisole (BHA), (3) DL-alpha-tocopherol, (4) ascorbyl palmitate and (5) propyl gallate. A pharmaceutical composition can include a mixture of two, three, four or five antioxidants.

A concentration of an active ingredient in a liquid composition sometimes is from about 0.1 wt % to about 35 wt %, or sometimes from about 0.5 wt % to about 10 wt %. The concentration in a semi-solid or solid composition such as a gel or a powder sometimes is about 0.1 wt % to about 5 wt %, or sometimes about 0.5 wt % to about 2.5 wt %. Higher concentrations are also appropriate for some solid or semi-solid compositions and may include amounts up to about 25 wt % or up to about 50 wt % or more.

A pharmaceutical composition may be prepared according to conventional techniques known in the pharmaceutical industry. In general terms, such techniques include bringing an active ingredient into association with on or more pharmaceutical carrier(s) and/or excipient(s) in liquid form or finely divided solid form, or both, and then shaping the product if required.

A pharmaceutical composition may be incorporated into a suitable dosage form (for example, unit dosage form), non-limiting examples of which include a tablet, capsule, gel capsule, liquid syrup, soft gel, suppository, enema, dressing or device (for example, in a syringe (for example, auto-injection device) or microneedle device). A pharmaceutical composition may be formulated as a suspension in aqueous, non-aqueous, or mixed media. Aqueous suspensions may further contain substances that increase viscosity, including for example, sodium carboxymethylcellulose, sorbitol, and/or dextran. A suspension may also contain one or more stabilizers. An amount of active ingredient required for use in treatment will vary not only with the particular form selected (for example, the salt selected) but also with route of administration, the nature of the condition being treated and the age and condition of the patient and ultimately will be at the discretion of the attendant physician or clinician. Any additive and material used in preparing a unit dosage form typically is pharmaceutically acceptable and substantially non-toxic in the amounts employed.

A composition can include a pharmaceutically acceptable ester or amide of a compound herein. In certain implementations, a composition includes a pharmaceutically acceptable salt of a compound herein. Non-limiting examples of pharmaceutically acceptable salts include carboxylate salts, amino acid addition salts and zwitterionic forms thereof, which are known to those skilled in the art as suitable for use with humans and animals. (See, for example, Gerge, S. M., et al, “Pharmaceutical Salts,” Pharm. Sci. (1977) 66:1-19). In cases where a compound is sufficiently basic or acidic to form a stable nontoxic acid or base salt, a composition includes a pharmaceutically acceptable salt of the compound. Non-limiting examples of pharmaceutically acceptable salts are organic acid addition salts formed with acids that form a physiological acceptable anion, non-limiting examples of which include tosylate, methanesulfonate, acetate, citrate, malonate, tartarate, succinate, benzoate, ascorbate, [alpha]-ketoglutarate, and [alpha]-glycerophosphate. Suitable inorganic salts may also be formed, including hydrochloride, sulfate, nitrate, bicarbonate, and carbonate salts. Pharmaceutically acceptable salts are obtained using standard procedures known in the art. For example, pharmaceutically acceptable salts may be obtained by reacting a sufficiently basic compound with a suitable acid affording a physiologically acceptable anion. Alkali metal (for example, sodium, potassium or lithium) or alkaline earth metal (for example, calcium, magnesium) salts of carboxylic acids and other anionic groups in molecules within a pharmaceutical composition also are contemplated.

A composition can include an isomer of a compound herein. Non-limiting examples of isomers are stereoisomers (e.g., diastereomers and enantiomers) and structural isomers such as tautomers. A composition can include a mixture containing two or more isomers of a compound herein. In certain instances, a mixture can include an isomer that predominates over one or more other isomers of a compound herein (e.g., the molar amount of one isomer may represent about 55% or more of all isomers of the compound (e.g., about 60% or more, about 65% or more, about 70% or more, about 75% or more, about 80% or more, about 85% or more)). A composition can include an isomerically pure form of a compound herein, in which the molar amount of one isomer can represent about 95% or more (e.g., about 96% or more, about 97% or more, about 98% or more, about 99% or more or about 99.5% or more) of all isomers of the compound.

Non-limiting examples of pharmaceutical compositions are provided hereafter.

### Pharmaceutical Composition for Oral Administration

A pharmaceutical composition may be provided as a tablet (for example, ingestible tablet, buccal tablet), troche, capsule (for example, hard- or soft-shell gelatin capsule), drink, elixir, suspension, syrup, wafer, and the like, and/or may be incorporated directly in food or drink that is part of a subject's diet. Such compositions and preparations sometimes contain at least 0.1% of active ingredient. The percentage of the compositions and preparations may be varied and sometimes are about 2% to about 60% of the weight of a given unit dosage form. The amount of active ingredient in a pharmaceutical composition is such that an effective dosage level can be obtained.

Tablets, troches, pills, capsules, and the like may contain one or more of the following: a binder such as gum tragacanth, acacia, corn starch or gelatin; an excipient such as dicalcium phosphate; a disintegrating agent such as corn starch, potato starch, alginic acid and the like; a lubricant such as magnesium stearate; a sweetening agent such as sucrose, fructose, lactose or aspartame; a flavoring agent such as peppermint, oil of wintergreen, or cherry flavoring. When the unit dosage form is a capsule, it may contain, in addition to materials described above, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor.

### Pharmaceutical Composition for Topical Administration

For topical administration, a compound herein may be applied in liquid form. A compound herein may be combined with a dermatologically acceptable carrier, which may be a solid or a liquid. A compound herein may be formulated with a solid carrier, which include finely divided solids such as talc, clay, microcrystalline cellulose, silica, alumina and the like. Useful liquid carriers include water, alcohols or glycols or water-alcohol/glycol blends, or phospholipids in propylene glycol/ethylene glycol, in which the a compound herein can be dissolved or dispersed at an effective level, optionally with the aid of non-toxic surfactants. A composition sometimes includes a diluent and sometimes a carrier (for example, assimilable, editable), buffer, preservative and the like. Additives such as fragrances and additional antimicrobial agents can be added to optimize the properties for a given use. A liquid composition can be applied from an absorbent pad, used to impregnate a bandage or other dressing, or sprayed onto the affected area using a pump-type or aerosol sprayer. A thickener, such as a synthetic polymer, fatty acid, fatty acid salt and/or ester, fatty alcohol, modified cellulose or modified mineral material, can also be employed with a liquid carrier to form a spreadable cream, paste, gel, ointment, soap, and the like, for application directly to the skin of a subject.

### Pharmaceutical Composition for Administration by Injection

A pharmaceutical composition suitable for injection (for example, subcutaneous, intramuscular, intravenous administration) can include a sterile aqueous solution or dispersion or sterile powder for the extemporaneous preparation of a sterile injectable solution or dispersion. An injectable formulation often is sterile and often is fluid. It typically is stable under the conditions of manufacture and storage and typically is preserved against contaminating microorganisms, such as bacteria and fungi. A pharmaceutical composition can be delivered to a subject via any suitable injection device, including without limitation, a syringe, needle or microneedle (for example, including a syringe device for self-administration; auto-injection device).

An injectable formulation sometimes includes a carrier, which can be a solvent, excipient, or dispersion medium. A liquid carrier or vehicle can be a solvent or liquid dispersion medium including, for example, water, ethanol, a polyol (for example, glycerol, propylene glycol, liquid polyethylene glycols, and the like), vegetable oil, nontoxic glyceryl ester, or suitable mixture thereof. Fluidity of an injectable formulation can be maintained, for example, by the use of a coating, such as lecithin, by the maintenance of the required particle size in the case of dispersion and by the use of a surfactant. Prevention of the action of microorganisms can be affected by an antibacterial and/or antifungal agent, for example, paraben, chlorobutanol, phenol, sorbic acid, thimerosal, and the like. In certain instances, an isotonic agent may be included, for example, a sugar or sodium chloride. Prolonged absorption of an injectable composition can be affected by use of an absorption delaying agent, for example, aluminum monostearate and/or gelatin. A pharmaceutical composition may include a co-polymer such as, for example, a co-polymer selected from poly(vinyl alcohol), poly(vinyl pyrrolidone), and hypromellose acetate succinate.

A sterile injectable solution can be prepared by incorporating an active ingredient in the required amount in the appropriate solvent with one or more other ingredients, as required, followed by filter sterilization. In the case of a sterile powder for the preparation of a sterile injectable solution, methods of preparation include vacuum drying and freeze-drying techniques, which yield a powder including the active ingredient and any additional desired ingredient present in the previously sterile-filtered solution.

### Protein Kinase Inhibition

A compound can be an inhibitor of one or more protein kinases (PKs), and can be used to inhibit one or more PKs. A compound that inhibits a PK can bind to a PK, inhibit PK substrate phosphorylation activity and/or inhibit binding activity of a PK to another entity (for example, a polypeptide to which the PK binds).

A compound that is an inhibitor of one or more PKs sometimes is an effective inhibitor, moderate inhibitor, mild inhibitor and/or ineffective inhibitor of one or more PKs. A compound is designated as an “effective inhibitor” when (i) the compound at a concentration of 100 nM inhibits PK activity by greater than 90%; and/or (ii) the compound inhibits PK activity at a measured IC₅₀ value of less than 20 nM. A compound is designated as a “moderate inhibitor” when (i) the compound at a concentration of 100 nM inhibits PK activity by 70% to 90%; and/or (ii) the compound inhibits PK activity at a measured IC₅₀ value between 20 nM and 40 nM. A compound is designated as a “mild inhibitor” when (i) the compound at a concentration of 100 nM inhibits PK activity by 30% to 70%; and/or (ii) the compound inhibits PK activity at a measured IC₅₀ value between 40 nM and 200 nM. A compound is designated as an “ineffective inhibitor” when (i) the compound at a concentration of 100 nM inhibits PK activity by less than 30%; and/or (ii) the compound inhibits PK phosphorylation at a measured IC₅₀ value of greater than 200 nM. An “effective inhibitor” can be used to effectively inhibit a PK, a “moderate inhibitor” can be used to moderately inhibit a PK, a “mild inhibitor” can be used to mildly inhibit a PK, and an “ineffective inhibitor” can be used to ineffectively inhibit or not inhibit a PK. An IC₅₀ value or percent activity can be measured by a suitable assay, such as an in vitro assay that assesses PK phosphorylation activity or PK ligand binding, for example. In certain embodiments, PK inhibition is assessed according to an IC₅₀ value or percent activity measured by a peptide cleavage assay, tracer displacement assay, solid phase inhibitor competition assay, ADP formation assay, as described herein (described in Example 17), for example.

In certain embodiments, a compound that contains a quinazolinyl group and an amine-linked phenyl group can be used to inhibit one or more of, or two or more of, or three or more of, or four or more of, or five or more of, or six or more of, or seven or more of, or eight or more of, or nine or more of, or ten or more of, or eleven or more of, or twelve or more of, or thirteen or more of, or each of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

In certain embodiments, a compound that contains a quinazolinyl group and an amine-linked phenyl group can selectively inhibit a JAK2 PK. In certain instances, the compound effectively inhibits or moderately inhibits a JAK2(wt) PK and does not effectively inhibit and does not moderately inhibit a JAK (wt) PK (for example, the compound mildly inhibits or ineffectively inhibits a JAK1 PK). In certain instances, the compound inhibits JAK3(wt) PK, and optionally effectively inhibits JAK3(wt) PK, or optionally moderately inhibits a JAK3(wt) PK. In certain instances, the compound inhibits, and optionally effectively inhibits, one or more of, or two or more of, or three or more of, or four or more of, or five or more of, or six or more of, or seven or more of, or eight or more of, or nine or more of, or ten or more of, or eleven or more of, or twelve or more of, or each of: an ABL family PK, a BTK family PK, a AURK family PK, a TRK family PK, a RET PK, an EPH family PK, a TNK family PK, a PLK family PK, an IRAK family PK, a SRC family PK, a LCK family PK, a DDR family PK, and/or a PTK family PK.

In certain instances, a compound effectively inhibits two or more of, or three or more of, or four or more of, or five or more of, or six or more of, or seven or more of, or eight or more of, or each of: an ABL family PK, a BTK family PK, a AURK family PK, a JAK family PK, a TRK family PK, a RET PK, an EPH family PK, a TNK family PK, a PLK family PK, an IRAK family PK, SRC family PK, DDR family PK, and/or PTK family PK. In certain instances, the compound moderately inhibits two or more of, or three or more of, or four or more of, or five or more of, or six or more of, or seven or more of, or eight or more of, or each of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

In certain embodiments, a compound that effectively inhibits and/or optionally moderately inhibits a PK in one or more of the foregoing PK families, does not effectively inhibit, and/or does not moderately inhibit, one or more of, or two or more of, or three or more of, or four or more of, or five or more of, or six or more of, or seven or more of, or eight or more of, or nine or more of, or ten or more of, or eleven or more of, or twelve or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

In certain embodiments, a compound used to inhibit a PK is a compound herein (in Table A, for example). In certain instances, a composition used to inhibit a PK contains a compound herein (in Table A, for example).

A compound can be an effective inhibitor or moderate inhibitor of, and can be used to inhibit, an ABL family PK, including an ABL1 PK, including ABL1(wt) and/or one or more ABL1 variants; and/or an ABL2 PK, including ABL2(wt) and/or one or more ABL2 variants. An ABL1 variant can include one or more of the following amino acid substitutions (relative to ABL1(wt)): T315I, G250E, Q252H, Y253F, E255K, F317L, M351T and H396P, and/or other ABL1 amino acid substitution described herein. An ABL2 PK also is referred to as an ARG PK.

A compound can be an effective inhibitor or moderate inhibitor of, and can be used to inhibit, a BTK family PK. A BTK family PK can include: a BTK PK, including BTK(wt) and/or one or more BTK variants; a BMX PK, including BMX(wt) and/or one or more BMX variants; an ITK PK, including ITK(wt) and/or one or more ITK variants; a TEC PK, including TEC(wt) and/or one or more TEC variants; and a TXK PK, including TXK(wt) and/or one or more TXK variants. BMX is referred to also as ETK, ITK is referred to also as EMT and TXK is referred to also as RLK. In certain embodiments, a compound is an effective inhibitor or moderate inhibitor of TXK(wt) and/or one or more TXK variants, and sometimes is a compound of Subgroup 8.

A compound can be an effective inhibitor or moderate inhibitor of, and can be used to inhibit, an Aurora (AURK) family PK. An AURK family PK can include: an AURKA PK, including AURKA(wt) and/or one or more AURKA variants; an AURKB PK, including AURKB(wt) and/or one or more AURKB variants; and an AURKC PK, including AURKC(wt) and/or one or more AURKC variants.

A compound can be an effective inhibitor or moderate inhibitor of, and can be used to inhibit, a JAK family PK. A JAK family PK can include: a JAK1 PK, including JAK1(wt) and/or one or more JAK1 variants; a JAK2 PK, including JAK2(wt) and/or one or more JAK2 variants; a JAK3 PK including JAK3(wt) and/or one or more JAK3 variants; and a TYK family PK. A TYK family PK can be a TYK2 family PK, including TYK2(wt) and/or one or more TYK2 variants. A JAK2 PK variant can include the amino acid substitution V617F, relative to JAK2(wt).

In certain embodiments, a compound is an effective inhibitor or moderate inhibitor of, and can be used to inhibit, a TYK family PK. In certain instances, a compound is an effective inhibitor or moderate inhibitor of, and can be used to inhibit, TYK2(wt). In certain embodiments, the compound does not effectively inhibit one or more of, or two or more of, or each of JAK1(wt), JAK2(wt) and JAK3(wt), and optionally does not moderately inhibit one or more of, or two or more of, or each of JAK1(wt), JAK2(wt) and/or JAK3(wt). In certain embodiments the compound is a compound of Subgroup 10.

In certain embodiments, a compound is an effective inhibitor or moderate inhibitor of JAK2(wt) and optionally one or more JAK2 variants. In certain instances, a compound is an effective inhibitor or moderate inhibitor of JAK3(wt) and optionally one or more JAK3 variants. In certain embodiments, a compound is a selective inhibitor of a JAK2 PK and does not effectively inhibit JAK (wt). In certain embodiments, a compound is a selective inhibitor of a JAK2 PK and does not moderately inhibit JAK (wt). In certain instances, a compound: does not effectively inhibit or moderately inhibit one or more JAK1 variants; does not effectively inhibit ABL1(wt) and/or ABL1(T315I); mildly inhibits ABL1(wt) and/or ABL1(T315I); ineffectively inhibits ABL1(wt) and/or ABL1(T315I); effectively inhibits ABL1(wt) and effectively inhibiting ABL1(T315I); moderately inhibits ABL1(wt); does not effectively inhibit, or optionally does not moderately inhibit, one or more of, or two or more of, or three or more of, or four or more of or five, or more of: an ABL family PK, a BTK family PK, a AURK family PK, a TRK family PK, a RET PK, an EPH family PK, a TNK family PK, a PLK family PK, an IRAK family PK, a SRC family PK, a LCK family PK, a DDR family PK, and/or a PTK family PK. In certain embodiments, a compound used to selective inhibit a JAK2 PK is a compound of Subgroup 2, Subgroup 3, Subgroup 4, Subgroup 6, Subgroup 7 or Subgroup 8. In certain embodiments, a compound inhibits, and optionally effectively inhibits or moderately inhibits, JAK2(wt) and a JAK2 variant comprising the V617F substitution, and in certain instances, the compound is a compound of Subgroup 3 or Subgroup 7.

A compound sometimes is an effective inhibitor or moderate inhibitor of, and can be used to inhibit, a TRK family PK. A TRK family PK can include: a TRKA PK, including TRKA(wt) and/or one or more TRKA variants; a TRKB PK, including TRKB(wt) and/or one or more TRKB variants; a TRKC PK, including TRKC(wt) and/or one or more TRKC variants; and a ROS1 PK, including ROS1(wt) and/or one or more ROS1 variants. TRKA is referred to also as NTRK1, TRKB is referred to also as NTRK2, and TRKC is referred to also as NTRK3.

A compound sometimes is an effective inhibitor or moderate inhibitor of, and can be used to inhibit, a RET family PK. A RET family PK can include: a RET PK, including RET(wt) and/or one or more RET variants. A RET variant can include one or more of the following amino acid substitutions, relative to RET(wt): A883F, G691S, M918T, S891A, V804E, V804L, V804M and Y791F.

A compound sometimes is an effective inhibitor or moderate inhibitor of, and can be used to inhibit, an EPH family PK. An EPH family PK can include an EPHA1 PK, including EPHA1(wt) and/or one or more EPHA1 variants; an EPHA2 PK, including EPHA2(wt) and/or one or more EPHA2 variants; an EPHA5 PK, including EPHA5(wt) and/or one or more EPHA5 variants; an EPHA8 PK, including EPHA8(wt) and/or one or more EPHA8 variants; an EPHB1 PK, including EPHB1(wt) and/or one or more EPHB1 variants; and/or an EPHB2 PK, including EPHB2(wt) and/or one or more EPHB2 variants.

A compound sometimes is an effective inhibitor or moderate inhibitor of, and can be used to inhibit, a TNK family PK. A TNK family PK can include a TNK1 PK, including TNK1(wt) and/or one or more TNK1 variants; and/or a TNK2 PK, including TNK2(wt) and/or one or more TNK2 variants. A TNK2 PK sometimes is referred to as an ACK PK.

A compound sometimes is an effective inhibitor or moderate inhibitor of, and can be used to inhibit, a PTK family PK. A PTK family PK can include a PTK2B PK, including PTK2B(wt) and/or one or more PTK2B variants. A PTK2B PK can be referred to as a FAK2 PK.

A compound sometimes is an effective inhibitor or moderate inhibitor of, and can be used to inhibit, a SRC family PK. A SRC family PK can include a SRC(wt) and/or one or more SRC variants, and a SRC-N1(wt) and/or one or more SRC-N1 variants. A compound sometimes is an effective inhibitor or moderate inhibitor of, and can be used to inhibit, a LCK family PK. A LCK family PK can include a LCK(wt) and/or one or more LCK variants.

A compound sometimes is an effective inhibitor or moderate inhibitor of, and can be used to inhibit, a DDR family PK. A DDR family PK can include a DDR2 PK, including DDR2(wt) and/or one or more DDR2 variants. A DDR variant PK can include a T654M and/or N456S amino acid substitution.

A compound sometimes is an effective inhibitor or moderate inhibitor of, and can be used to inhibit, a PLK family PK. A PLK family PK can include PLK4(wt) and/or one or more PLK4 variants. A compound sometimes is an effective inhibitor or moderate inhibitor of, and can be used to inhibit, an IRAK family PK. An IRAK family PK can include an IRAK1 PK, including IRAK1(wt) and/or one or more IRAK1 variants; and/or an IRAK3 PK, including IRAK3(wt) and/or one or more IRAK3 variants. In certain embodiments, a compound used to inhibit a PLK PK and/or IRAK PK is a compound of Subgroup 9.

A compound sometimes is an effective inhibitor or moderate inhibitor of, and can be used to inhibit, one or more, or two or more, variants of a PK. In certain embodiments, a compound is an effective inhibitor or moderate inhibitor of, and can be used to inhibit, one or more of the following PK variants: (i) an ABL1 variant optionally containing one or more of the following amino acid substitutions relative to ABL1(wt): T315I, G250E, Q252H, Y253F, E255K, F317L, M351T and H396P; (ii) a JAK2 PK variant optionally containing the amino acid substitution V617F relative to JAK2(wt); (iii) a RET variant optionally containing one or more of the following amino acid substitutions relative to RET(wt): A883F, G691S, M918T, S891A, V804E, V804L, V804M and Y791F; and/or (iv) a DDR2 variant optionally containing the amino acid substitution T654M relative to DDR2(wt) and/or the amino acid substitution N456S relative to DDR2(wt). A PK variant can be referred to with the PK name as a prefix and an amino acid substation as a suffix, where the amino acid substation suffix is separated from the PK name prefix by a hyphen (for example, ABL1-T315I or DDR2-N456S) or by parentheses (for example, ABL1(T315I) or DDR2(N456S)).

A compound can be an effective inhibitor of, and can be used to inhibit, two or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK. A compound can be an effective inhibitor of, and can be used to inhibit, an ABL family PK and a BTK family PK (for example, a BTK(wt) PK). A compound can be an effective inhibitor of, and can be used to inhibit, an ABL family PK and a AURK family PK. A compound can be an effective inhibitor of, and can be used to inhibit, an ABL family PK and a JAK family PK (for example, JAK2(wt) and/or JAK2(V617F)).

A compound can be an effective inhibitor of, and can be used to inhibit, three or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK. A compound can be an effective inhibitor of an ABL family PK (for example, ABL1(wt) and/or ABL1(T315I)), a BTK family PK (for example, a BTK(wt) PK) and a AURK family PK, for example.

A compound can be an effective inhibitor of, and can be used to inhibit, four or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK. A compound can be an effective inhibitor of: an ABL family PK (for example, ABL1(wt) and/or ABL1(T315I)), a BTK family PK (for example, a BTK(wt) PK), a AURK family PK, and a JAK family PK. A compound can be an effective inhibitor of: an ABL family PK (for example, ABL1(wt) and/or ABL1(T315I)), a BTK family PK (for example, a BTK(wt) PK), a AURK family PK, a JAK family PK (for example, JAK2(wt) PK and/or JAK2 variant PK containing a V617F amino acid substitution), and a TRK family PK.

A compound can be an effective inhibitor of, and can be used to inhibit, five of, or six of, or seven of, or eight of, or nine of, or ten of, or eleven of, or twelve of, or thirteen of or all of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

A compound can be an effective inhibitor or moderate inhibitor of ABL1, ABL1-T315I, AURKA, and JAK2. The compound can be an effective inhibitor or moderate inhibitor of AURKB. The compound can be an effective inhibitor or moderate inhibitor of BTK. The compound can be an effective inhibitor of JAK3. The compound can be an effective inhibitor or moderate inhibitor of JAK1, or the compound can be a mild inhibitor or ineffective inhibitor of JAK1. The compound can be an effective inhibitor or moderate inhibitor of one or more or all of TYK2, a TRK family PK, ROS1, TXK, an EPH family PK, IRAK3, PLK4, TNK1, RET and JAK2-V617F. The compound can be an effective inhibitor or moderate inhibitor of one or more or all of TRKA, TRKB, TRKC, ROS1, EPHA1 and EPHB1. The compound can be an effective inhibitor or moderate inhibitor of a SRC family PK and/or a DDR family PK. The compound can be an effective inhibitor of or moderate inhibitor of a DDR2 PK and optionally a DDR2 variant PK (for example, a DDR2 variant PK containing a T654M and/or N456S amino acid substitution). In certain embodiments the compound is an effective inhibitor of SRC(wt), SRC-N1 (wt), and a DDR2 variant (DDR2-T654M for example). In certain instances the compound is a mild inhibitor of SRC(wt), a moderate inhibitor of SRC-N1 (wt), and an effective inhibitor of a DDR2 variant (DDR2-T654M for example). The compound can be an effective inhibitor or moderate inhibitor of ABL2. The compound can be an effective inhibitor or moderate inhibitor of PTK2B. The compound can be an effective inhibitor or moderate inhibitor of ABL1, ABL1-T315I, ABL2, TYK2, JAK2, TRKC and PTK2B. The compound can be an effective inhibitor or moderate inhibitor of ABL1, ABL1-T315I, BTK, AURKA, JAK2, NTRK3, PTK2B, TYK2 and ABL2. In certain instances, the NTRK3 is a ETV6-NTRK3 fusion. The compound can be an effective inhibitor or moderate inhibitor of LCK. In certain instances, the compound is a Subgroup 1 or Subgroup 5 compound.

A compound of Subgroup 1 and/or Subgroup 5 can be an effective inhibitor of, or optionally a moderate inhibitor of, and can be used to inhibit, multiple PTKs, including ten or more of, or eleven or more of, or twelve or more of, or thirteen or more of, or all of an ABL family PK, a BTK family PK, an AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

A compound can be an effective inhibitor of JAK2, JAK3, JAK2-V617F and a mild inhibitor of JAK1. The compound can be an effective inhibitor of or a moderate inhibitor of one or more or all of ABL1, TRKA, TRKB, TRKC, ROS1, AURKA, IRAK3, TNK1. In certain instances, the compound is a Subgroup 6 compound.

A compound can be an effective inhibitor of JAK2, an ineffective inhibitor of or a mild inhibitor of JAK1, and optionally: a moderate inhibitor of JAK3 and/or a mild inhibitor of JAK2-V617F. The compound can be an effective inhibitor of or a moderate inhibitor of one or more or all of TRKB, TRKC, AURKA, IRAK3 and PLK4. In certain instances, the compound is a Subgroup 4 compound.

A compound can be an effective inhibitor of JAK2, an ineffective inhibitor of or a mild inhibitor of JAK1, and optionally: a moderate inhibitor of JAK3 and/or JAK2-V617F. The compound can be an effective inhibitor of or a moderate inhibitor of one or more or all of TXK, JAK2-V617F, TRKB, TRKC, ROS1, AURKA, IRAK3 and TNK1. In certain instances, the compound is a Subgroup 2 compound.

A compound can be an effective inhibitor of JAK2, an ineffective inhibitor of or a mild inhibitor of JAK1, and optionally: an effective inhibitor of JAK3 and/or JAK2-V617F. In certain instances, the compound is a Subgroup 3 compound.

A compound can be an effective inhibitor of JAK2, a moderate inhibitor of JAK1, and optionally: a moderate inhibitor of JAK3 and/or a moderate inhibitor of or an effective inhibitor of JAK2-V617F. In certain instances, the compound is a Subgroup 7 compound.

A compound can be an effective inhibitor of or a moderate inhibitor of TYK2. The compound can be an effective inhibitor of or a moderate inhibitor of JAK3. The compound can be a mild inhibitor of or an ineffective inhibitor of JAK1 and/or JAK2. The compound can be an effective inhibitor of or a moderate inhibitor of one or more or all of ABL1-T315I, AURKA, PLK4 and IRAK3. In certain instances, the compound is a Subgroup 10 compound.

A compound can be an effective inhibitor of or a moderate inhibitor of TXK. The compound can be an effective inhibitor of JAK2, an ineffective inhibitor of or a mild inhibitor of JAK1, and optionally: for an effective inhibitor of or a moderate inhibitor of JAK3 and/or JAK2-V617F. In certain instances, the compound is a Subgroup 8 compound.

A compound can be an effective inhibitor of or a moderate inhibitor of IRAK3. A compound can be an effective inhibitor of or a moderate inhibitor of PLK4. In certain instances, the compound is a Subgroup 9 compound.

A compound can inhibit a Homo sapiens PK or viral PK. A polypeptide of each of the foregoing wild type (wt) Homo sapiens PKs is accessible in (i) a public database (World Wide Web URL ncbi.nlm.nih.gov/protein/) according to the corresponding accession number having a “NP” or “AA” prefix, and (ii) a public database (World Wide Web URL uniprot.org/uniprotkb/) according to the corresponding accession number having a “P” prefix, in Table 4 of Example 17. A polypeptide of a corresponding PK variant can be determined according to the wt polypeptide accessed from the database and the position of one or more amino acid substitutions designated.

JAK (wt), JAK2(wt) and JAK3(wt) are receptor PKs. The JAK (wt) catalytic domain includes amino acids 866-1154 of the JAK (wt) polypeptide accessed by the accession number in Table 4 of Example 17, which can be utilized in assays. The JAK2(wt) catalytic domain includes amino acids 808-1132 of the JAK2(wt) polypeptide accessed by the accession number in Table 4 of Example 17, which can be utilized in assays. The JAK3(wt) catalytic domain includes amino acids 781-1124 of the JAK3(wt) polypeptide accessed by the accession number in Table 4 of Example 17, which can be utilized in assays.

TRKA(wt), TRKB(wt) and TRKC(wt) also are receptor PKs. The TRKA(wt) catalytic domain includes amino acids 441-796 of the TRKA(wt) polypeptide accessed by the accession number in Table 4 of Example 17, which can be utilized in assays. The TRKB(wt) catalytic domain includes amino acids 526-838 of the TRKB(wt) polypeptide accessed by the accession number in Table 4 of Example 17, which can be utilized in assays. The TRKC(wt) catalytic domain includes amino acids 510-825 of the TRKC(wt) polypeptide accessed by the accession number in Table 4 of Example 17, which can be utilized in assays. RET(wt) also is a receptor PK and the catalytic domain includes amino acids 658-1114 of the polypeptide accessed by the accession number in Table 4 of Example 17, which can be utilized in assays.

EPHA1(wt), EPHA2(wt), EPHA5(wt), EPHA8(wt), EPHB1(wt) and EPHB2(wt) PKs also are receptor PKs. The EPHA1(wt) catalytic domain includes amino acids 568-976 of the EPHA1(wt) polypeptide accessed by the accession number in Table 4 of Example 17, which can be utilized in assays. The EPHA2(wt) catalytic domain includes amino acids 560-976 of the EPHA2(wt) polypeptide accessed by the accession number in Table 4 of Example 17, which can be utilized in assays. The EPHA5(wt) catalytic domain includes amino acids 595-1037 of the EPHA5(wt) polypeptide accessed by the accession number in Table 4 of Example 17, which can be utilized in assays. The EPHA8(wt) catalytic domain includes amino acids 565-1005 of the EPHA8(wt) polypeptide accessed by the accession number in Table 4 of Example 17, which can be utilized in assays. The EPHB1(wt) catalytic domain includes amino acids 612-887 of the EPHB1(wt) polypeptide accessed by the accession number in Table 4 of Example 17, which can be utilized in assays. The EPHB2(wt) catalytic domain includes amino acids 616-884 of the EPHB2(wt) polypeptide accessed by the accession number in Table 4 of Example 17, which can be utilized in assays.

TYK2(wt) also is a receptor PK and the catalytic domain includes amino acids 833-1187 of the polypeptide accessed by the accession number in Table 4 of Example 17, which can be utilized in assays. TNK2(wt), which also is referred to as ACK, also is a receptor PK and the catalytic domain includes amino acids 110-476 of the polypeptide accessed by the accession number in Table 4 of Example 17, which can be utilized in assays. IRAK1(wt) also is a receptor PK and the catalytic domain includes amino acids 194-712 of the polypeptide accessed by the accession number in Table 4 of Example 17, which can be utilized in assays. DDR2 also is a receptor PK and the catalytic domain includes amino acids 422-855 of the polypeptide accessed by the accession number in Table 4 of Example 17, which can be utilized in assays.

The SRC(wt) polypeptide can be accessed by either of the accession numbers shown in Table 4 of Example 17. The SRC-N1(wt) polypeptide is nearly identical to the SRC(wt) polypeptide but includes a 6-amino acid polypeptide insertion in the SH3 domain of the SRC(wt) polypeptide. The T at position 117 in the SRC(wt) polypeptide is replaced by TRKVDVR in the SRC-N1(wt) polypeptide.

A PK referred to herein as ABL1, ABL2, AURKA, AURKB, AURK3, BTK, BMX, ITK, TEC, TXK, JAK1, JAK2, JAK3, TYK2, ROS1, EPHA1, EPHA2, EPHA5, EPHA8, EPHB1, EPHB2, IRAK1, IRAK3, PLK4, TNK1, TNK2, RET, TRKA, TRKB, TRKC, NTRK1, NTRK2, NTRK3, SRC, SRC-N1, LCK, DDR2 or PTK2B, without an indication of an amino acid substitution, typically contains the polypeptide of ABL1(wt), ABL2(wt), AURKA(wt), AURKB(wt), AURK3(wt), BTK(wt), BMX(wt), ITK(wt), TEC(wt), TXK(wt), JAK (wt), JAK2(wt), JAK3(wt), TYK2(wt), ROS1(wt), EPHA1(wt), EPHA2(wt), EPHA5(wt), EPHA8(wt), EPHB1(wt), EPHB2(wt), IRAK1(wt), IRAK3(wt), PLK4(wt), TNK1(wt), TNK2(wt), RET(wt), TRKA(wt), TRKB(wt), TRKC(wt), NTRK1(wt), NTRK2(wt), NTRK3(wt), SRC(wt), SRC-N1 (wt), LCK(wt), DDR2(wt) or PTK2B(wt), respectively, or a portion thereof containing (i) a catalytic domain or (ii) a fragment of a catalytic domain having phosphoryl-transfer activity (for example, in vitro phosphoryl-transfer activity). A PK referred to as having an amino acid substitution herein (for example DDR2-N456S and/or DDR2-T654M) typically includes the designated amino acid substitution at the designated position in the polypeptide accessed by the accession number referenced in Table 4 of Example 17, and can be the length of the accessed polypeptide, or a portion thereof containing (i) a catalytic domain or (ii) a fragment of a catalytic domain having phosphoryl-transfer activity (for example, in vitro phosphoryl-transfer activity).

### ABL1 Inhibition

In certain embodiments, a compound is an effective inhibitor or moderate inhibitor of one or more, or two or more, ABL1 variant polypeptides. Such a compound can be an effective inhibitor or moderate inhibitor of ABL1(wt). A compound that is an effective inhibitor or two or more ABL1 variant polypeptides can be considered a pan-ABL1 inhibitor. In certain embodiments, a compound is an effective inhibitor of two or more ABL1 variant polypeptides containing one or more of M244V, G250E, Q252H, Y253F, Y253H, E255K, E255V, V299L, F311L, T315A, T315I, F317L, F317V, M351T, E355G, F359V, V379I, L387M, H396P and/or H396R. A compound sometimes is an effective inhibitor of ABL1(T315I) and an effective inhibitor of one or more other ABL1 variant polypeptides (for example, two or more other ABL1 variant polypeptides, three or more other ABL1 variant polypeptides, or four or more other ABL1 variant polypeptides). A compound sometimes is an effective inhibitor of ABL1(T315I) and an effective inhibitor of one or more of ABL1(G250E), ABL1(Y253F), ABL1(E255K) and ABL1(F317L). A compound sometimes is an effective inhibitor of ABL1(T315I) and an effective inhibitor of two or more of ABL1(G250E), ABL1(Y253F), ABL1(E255K) and ABL1(F317L). A compound sometimes is an effective inhibitor of ABL1(T315I) and an effective inhibitor of three or more of ABL1(G250E), ABL1(Y253F), ABL1(E255K) and ABL1(F317L). A compound sometimes is an effective inhibitor of ABL1(T315I), an effective inhibitor one or more other ABL1 variant polypeptides, and a moderate inhibitor of one or more other ABL1 variant polypeptides. A compound sometimes is an effective inhibitor of ABL1(T315I), an effective inhibitor of one or more of ABL1(G250E), ABL1(Y253F), ABL1(E255K) and ABL1(F317L), and a moderate inhibitor of one or more of ABL1(G250E), ABL1(Y253F), ABL1(E255K) and ABL1(F317L). A compound sometimes is an effective inhibitor of ABL1(T315I), ABL1(G250E), ABL1(Y253F) and ABL1(E255K), and a moderate inhibitor of ABL1(F317L).

MLEICLKLVGCKSKKGLSSSSSCYLEEALORPVASDEEPOGLSEAARWNSKENLLAGPSENDPN
LFVALYDFVASGDNTLSITKGEKLRVLGYNHNGEWCEAQTKNGOGWVPSNYITPVNSLEKHSWY
HGPVSRNAAEYLLSSGINGSELVRESESSPGORSISLRYEGRVYHYRINTASDGKLYVSSESRF
NTLAELVHHHSTVADGLITTLHYPAPKRNKPTVYGVSPNYDKWEMERTDITMKHKLGGGQYGEV
YEGVWKKYSLTVAVKTLKEDTMEVEEFLKEAAVMKEIKHPNLVQLLGVCTREPPFYIITEFMTY
GNLLDYLRECNRQEVNAVVLLYMATQISSAMEYLEKKNFIHRDLAARNCLVGENHLVKVADFGL
SRLMTGDTYTAHAGAKFPIKWTAPESLAYNKFSIKSDVWAFGVLLWEIATYGMSPYPGIDLSQV
YELLEKDYRMERPEGCPEKVYELMRACWQWNPSDRPSFAEIHQAFETMFQESSISDEVEKELGK

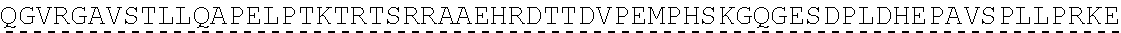

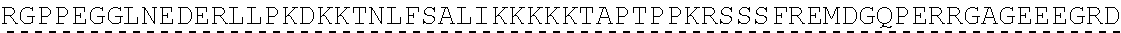

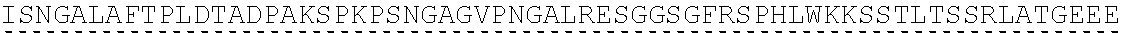

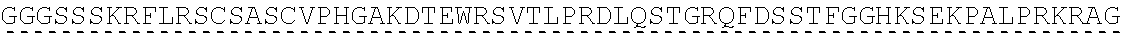

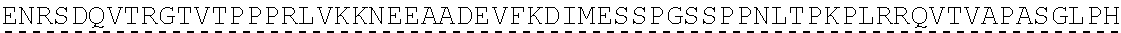

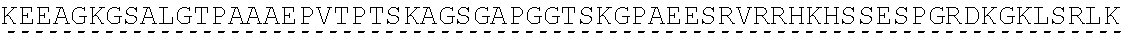

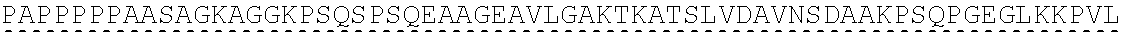

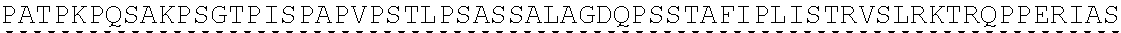

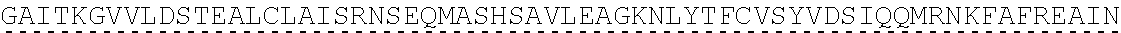

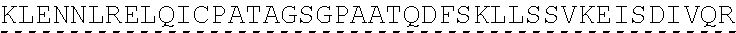

The following is the ABL1 “isoform b” polypeptide (SEQ ID NO: 2).
MGQQPGKVLGDQRRPSLPALHFIKGAGKKESSRHGGPHCNVFVEHEALORPVASDEEPOGLSEA
ARWNSKENLLAGPSENDPNLFVALYDFVASGDNILSITKGEKLRVLGYNHNGEWCEAQTKNGQG
WVPSNYITPVNSLEKHSWYHGPVSRNAAEYLLSSGINGSELVRESESSPGORSISLRYEGRVYH
YRINTASDGKLYVSSESRENTLAELVHHHSTVADGLITTLHYPAPKRNKPTVYGVSPNYDKWEM
ERTDITMKHKLGGGQYGEVYEGVWKKYSLTVAVKTLKEDTMEVEEFLKEAAVMKEIKHPNLVQL
LGVCTREPPFYIITEFMTYGNLLDYLRECNRQEVNAVVLLYMATQISSAMEYLEKKNFIHRDLA
ARNCLVGENHLVKVADFGLSRLMTGDTYTAHAGAKFPIKWTAPESLAYNKESIKSDVWAFGVLL
WEIATYGMSPYPGIDLSQVYELLEKDYRMERPEGCPEKVYELMRACWQWNPSDRPSFAEIHQAF

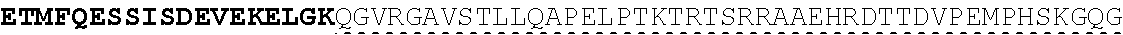

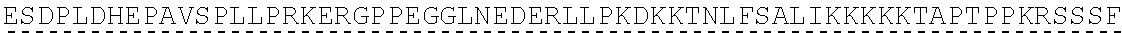

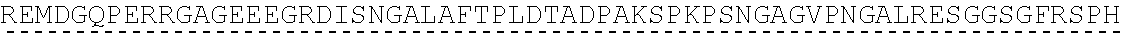

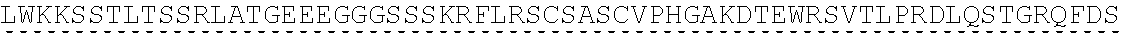

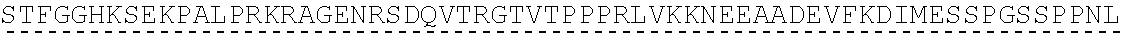

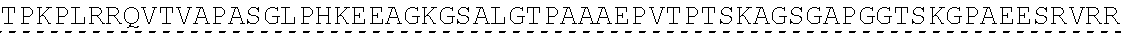

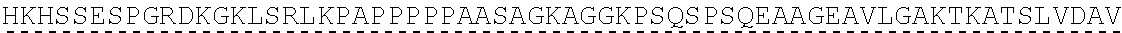

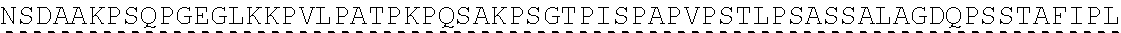

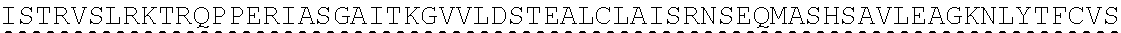

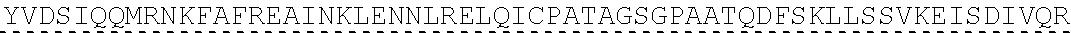

In each of the ABL1 “isoform a” polypeptide and ABL1 “isoform b” polypeptide depicted
above, an N-terminal region is underlined with single-underlining, followed by an adjacent
downstream region underlined with double-underlining, followed by a catalytic domain
highlighted in bold text, followed by a C-terminal region underlined with hatched
underlining. The ABL1 “isoform a” polypeptide N-terminal region underlined above by
single underlining differs from the ABL1 “isoform b” polypeptide N-terminal region
underlined above by single underlining. The adjacent, downstream polypeptide region in
each of the ABL1 “isoform a” polypeptide and ABL1 “isoform b” polypeptide, which is not
underlined with single underlining, and which starts from the end of the N-terminal region
underlined by single underlining and ends at the C-terminus, is identical (the “identical
portion”). The common catalytic domain (also referred to as a “kinase domain”) highlighted
in bold text in the ABL1 “isoform a” polypeptide and ABL1 “isoform b” polypeptide above is
reproduced below (SEQ ID NO: 3).
SPNYDKWEMERTDITMKHKLGGGQYGEVYEGVWKKYSLTVAVKTLKEDTMEVEEFLKEAAVMKE
IKHPNLVQLLGVCTREPPFYIITEFMTYGNLLDYLRECNRQEVNAVVLLYMATQISSAMEYLEK
KNFIHRDLAARNCLVGENHLVKVADFGLSRLMTGDTYTAHAGAKFPIKWTAPESLAYNKFSIKS
DVWAFGVLLWEIATYGMSPYPGIDLSQVYELLEKDYRMERPEGCPEKVYELMRACWQWNPSDRP
SFAEIHQAFETMFQESSISDEVEKELGK

An ABL1 polypeptide referred to as an “ABL1 wild type” polypeptide (“ABL1(wt)”) can (1) contain the polypeptide of SEQ ID NO:3; or (2) contain the polypeptide of SEQ ID NO:1; or (3) contain the polypeptide of SEQ ID NO:2; or (4) contain the polypeptide of SEQ ID NO:3 and: (i) an adjacent N-terminal region containing 2 or more contiguous amino acids in the double-underlined region and/or single underlined region shown above in SEQ ID NO: 1 or SEQ ID NO:2, or (ii) an adjacent C-terminal region containing 2 or more contiguous amino acids in the hatched-underlined region shown above in SEQ ID NO:1 and SEQ ID NO:2, or a combination of (i) and (ii); or (5) contain a fragment of (1), (2), (3) or (4) containing 25 or more contiguous amino acids or 1100 or fewer contiguous amino acids; or (6) contain an ABL1 polypeptide portion containing a polypeptide of (1), (2), (3), (4) or (5) and a non-ABL1 polypeptide portion.

An ABL1(wt) polypeptide typically does not include a substitution of an amino acid in the identical portion, i.e., the portion of the ABL1 “isoform a” and “isoform b” polypeptides above not underlined by single-underlining. An ABL1(wt) polypeptide typically includes no amino acid insertion or amino acid deletion relative to the polypeptide of SEQ ID NO:1, SEQ ID NO:2 or SEQ ID NO:3.

A fragment sometimes includes 25 or more contiguous amino acids of the polypeptide of SEQ ID NO:1, SEQ ID NO:2 or SEQ ID NO:3 (for example, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 110 or more, 120 or more, 130 or more, 140 or more, 150 or more, 160 or more, 170 or more, 180 or more, 190 or more, 200 or more, 225 or more, 250 or more, 275 or more, 300 or more, 325 or more, 350 or more, 375 or more, 400 or more, 425 or more, 450 or more, 475 or more, 500 or more, 525 or more, 550 or more, 575 or more, 600 or more, 625 or more, 650 or more, 675 or more, 700 or more, 725 or more, 750 or more, 775 or more, 800 or more, 825 or more, 850 or more, 875 or more, 900 or more, 925 or more, 950 or more, 975 or more, 1000 or more, 1025 or more, 1050 or more, 1075 or more, or 1100 or more contiguous amino acids of the polypeptide of SEQ ID NO:1, SEQ ID NO:2 or SEQ ID NO:3). A fragment sometimes includes 1100 or fewer contiguous amino acids of the polypeptide of SEQ ID NO:1, SEQ ID NO:2 or SEQ ID NO:3 (for example, 50 or fewer, 55 or fewer, 60 or fewer, 65 or fewer, 70 or fewer, 75 or fewer, 80 or fewer, 85 or fewer, 90 or fewer, 95 or fewer, 100 or fewer, 110 or fewer, 120 or fewer, 130 or fewer, 140 or fewer, 150 or fewer, 160 or fewer, 170 or fewer, 180 or fewer, 190 or fewer, 200 or fewer, 225 or fewer, 250 or fewer, 275 or fewer, 300 or fewer, 325 or fewer, 350 or fewer, 375 or fewer, 400 or fewer, 425 or fewer, 450 or fewer, 475 or fewer, 500 or fewer, 525 or fewer, 550 or fewer, 575 or fewer, 600 or fewer, 625 or fewer, 650 or fewer, 675 or fewer, 700 or fewer, 725 or fewer, 750 or fewer, 775 or fewer, 800 or fewer, 825 or fewer, 850 or fewer, 875 or fewer, 900 or fewer, 925 or fewer, 950 or fewer, 975 or fewer, 1000 or fewer, 1025 or fewer, 1050 or fewer, or 1075 or fewer contiguous amino acids of the polypeptide of SEQ ID NO:1, SEQ ID NO:2 or SEQ ID NO:3).

A non-ABL1 polypeptide portion present in an ABL1(wt) polypeptide sometime is (i) an N-terminal portion, or is located closer to the N-terminus of the ABL1(wt) polypeptide than the ABL1 polypeptide portion; or (ii) a C-terminal portion, or is located closer to the C-terminus of the ABL1(wt) polypeptide than the ABL1 polypeptide portion; or (iii) a combination of (i) and (ii) where there are multiple non-ABL1 polypeptide portions. A non-ABL1 polypeptide portion present in an ABL1(wt) polypeptide sometimes contains a BCR portion (for example, a BCR portion of a BCR-ABL1 fusion polypeptide identified in cancer patients). A BCR portion present in an ABL1(wt) polypeptide can be an N-terminal portion, or can be located closer to the N-terminus of the ABL1(wt) polypeptide than the ABL1 polypeptide portion.

An ABL1(wt) polypeptide can be modified with one or more components that facilitate use of the polypeptide, including without limitation one or more of separating, purifying, isolating and detecting an ABL1 polypeptide, and measuring an activity of an ABL1 polypeptide (for example, binding activity of an ABL1 polypeptide to a test compound and/or binding agent; phosphorylation activity of an ABL1 polypeptide). In certain embodiments, an ABL1(wt) polypeptide can be modified to include a binding pair member (for example, biotin/avidin (or streptavidin), antibody/antigen), a luminescence molecule (for example, bioluminescence molecule; luciferase or portion thereof; nano-luciferase), fluorophore (for example, member or members of a fluorescence resonance energy transfer (FRET) pair), dye, particle (for example, nanoparticle), and the like, for example.

For embodiments in which the component is a polypeptide, the polypeptide may be a non-ABL polypeptide portion referenced herein. In certain instances, a non-ABL1 polypeptide portion is an N-terminal or C-terminal portion useful to immobilizing the ABL1 polypeptide to a solid phase. In certain embodiments, a non-ABL polypeptide portion contains a poly-histidine peptide portion (for example, a peptide containing 5-20, or 6-10, consecutive histidine amino acids) capable of associating with a transition metal-containing solid phase (for example, a solid phase containing Mn, Fe, Co, Ni or Cu). A non-ABL1 polypeptide portion sometimes contains one or both of a linker polypeptide portion and a cleavage recognition polypeptide portion. Multiple linker polypeptide portions are known and can be selected. Multiple cleavage recognition polypeptide portions also are known and can be selected for cleavage of an N-terminal portion or C-terminal portion from an ABL1 polypeptide under suitable cleavage conditions.

A non-limiting example of an ABL1 (wt) polypeptide containing the “isoform a”
polypeptide of SEQ ID NO: 1 and a non-ABL1 C-terminal portion (highlighted in
bold text) containing a poly-histidine peptide portion and linker portion,
referred to herein as “isoahABL1 (wt),” is as follows (SEQ ID NO: 4):
MLEICLKLVGCKSKKGLSSSSSCYLEEALQRPVASDFEPQGLSEAARWNSKENLLAGPSENDPN
LFVALYDFVASGDNTLSITKGEKLRVLGYNHNGEWCEAQTKNGQGWVPSNYITPVNSLEKHSWY
HGPVSRNAAEYLLSSGINGSFLVRESESSPGQRSISLRYEGRVYHYRINTASDGKLYVSSESRF
NTLAELVHHHSTVADGLITTLHYPAPKRNKPTVYGVSPNYDKWEMERTDITMKHKLGGGQYGEV
YEGVWKKYSLTVAVKTLKEDTMEVEEFLKEAAVMKEIKHPNLVQLLGVCTREPPFYIITEFMTY
GNLLDYLRECNRQEVNAVVLLYMATQISSAMEYLEKKNFIHRDLAARNCLVGENHLVKVADFGL
SRLMTGDTYTAHAGAKFPIKWTAPESLAYNKFSIKSDVWAFGVLLWEIATYGMSPYPGIDLSQV
YELLEKDYRMERPEGCPEKVYELMRACWQWNPSDRPSFAEIHQAFETMFQESSISDEVEKELGK
QGVRGAVSILLQAPELPTKTRISRRAAEHRDITDVPEMPHSKGQGESDPLDHEPAVSPLLPRKE
RGPPEGGLNEDERLLPKDKKTNLFSALIKKKKKTAPTPPKRSSSFREMDGQPERRGAGEEEGRD
ISNGALAFTPLDTADPAKSPKPSNGAGVPNGALRESGGSGFRSPHLWKKSSTLTSSRLATGEEE
GGGSSSKRFLRSCSASCVPHGAKDTEWRSVTLPRDLQSTGRQFDSSTFGGHKSEKPALPRKRAG
ENRSDQVTRGTVTPPPRLVKKNEEAADEVFKDIMESSPGSSPPNLTPKPLRRQVTVAPASGLPH
KEEAGKGSALGTPAAAEPVTPTSKAGSGAPGGTSKGPAEESRVRRHKHSSESPGRDKGKLSRLK
PAPPPPPAASAGKAGGKPSQSPSQEAAGEAVLGAKTKATSLVDAVNSDAAKPSQPGEGLKKPVL
PATPKPQSAKPSGTPISPAPVPSILPSASSALAGDQPSSTAFIPLISTRVSLRKTRQPPERIAS
GAITKGVVLDSTEALCLAISRNSEQMASHSAVLEAGKNLYTFCVSYVDSIQQMRNKFAFREAIN
KLENNLRELQICPATAGSGPAATQDFSKLLSSVKEISDIVQRLEACQLGTDDYDIPTTHHHHHH
A non-limiting example of an ABL1 (wt) polypeptide containing the “isoform b”
polypeptide of SEQ ID NO: 2 and a non-ABL1 N-terminal portion (highlighted in
bold text) containing a nano-luciferase portion and linker portion, referred
to herein as “isoblABL1 (wt),” is as follows (SEQ ID NO: 5):
MVFTLEDFVGDWRQTAGYNLDQVLEQGGVSSLFQNLGVSVTPIQRIVLSGENGLKIDIHVIIPY
EGLSGDQMGQIEKIFKVVYPVDDHHFKVILHYGTLVIDGVTPNMIDYFGRPYEGIAVEDGKKIT
VTGTLWNGNKIIDERLINPDGSLLFRVTINGVTGWRLCERILAGGSGGGGSGGGSSGGAIAMGQ
QPGKVLGDQRRPSLPALHFIKGAGKKESSRHGGPHCNVFVEHEALQRPVASDFEPQGLSEAARW
NSKENLLAGPSENDPNLFVALYDFVASGDNILSITKGEKLRVLGYNHNGEWCEAQTKNGQGWVP
SNYITPVNSLEKHSWYHGPVSRNAAEYLLSSGINGSFLVRESESSPGQRSISLRYEGRVYHYRI
NTASDGKLYVSSESRFNTLAELVHHHSTVADGLITTLHYPAPKRNKPTVYGVSPNYDKWEMERT
DITMKHKLGGGQYGEVYEGVWKKYSLTVAVKTLKEDTMEVEEFLKEAAVMKEIKHPNLVQLLGV
CTREPPFYIITEFMTYGNLLDYLRECNRQEVNAVVLLYMATQISSAMEYLEKKNFIHRDLAARN
CLVGENHLVKVADFGLSRLMTGDTYTAHAGAKFPIKWTAPESLAYNKFSIKSDVWAFGVLLWEI
ATYGMSPYPGIDLSQVYELLEKDYRMERPEGCPEKVYELMRACWQWNPSDRPSFAEIHQAFETM
FQESSISDEVEKELGKQGVRGAVSTLLQAPELPTKTRISRRAAEHRDTTDVPEMPHSKGQGESD
PLDHEPAVSPLLPRKERGPPEGGLNEDERLLPKDKKINLFSALIKKKKKTAPTPPKRSSSFREM
DGQPERRGAGEEEGRDISNGALAFTPLDTADPAKSPKPSNGAGVPNGALRESGGSGFRSPHLWK
KSSTLISSRLATGEEEGGGSSSKRFLRSCSASCVPHGAKDTEWRSVTLPRDLQSTGRQFDSSTF
GGHKSEKPALPRKRAGENRSDQVTRGTVTPPPRLVKKNEEAADEVFKDIMESSPGSSPPNLTPK
PLRRQVTVAPASGLPHKEEAGKGSALGTPAAAEPVTPTSKAGSGAPGGTSKGPAEESRVRRHKH
SSESPGRDKGKLSRLKPAPPPPPAASAGKAGGKPSQSPSQEAAGEAVLGAKTKATSLVDAVNSD
AAKPSQPGEGLKKPVLPATPKPQSAKPSGTPISPAPVPSTLPSASSALAGDQPSSTAFIPLIST
RVSLRKTRQPPERIASGAITKGVVLDSTEALCLAISRNSEQMASHSAVLEAGKNLYTFCVSYVD
SIQQMRNKFAFREAINKLENNLRELQICPATAGSGPAATQDESKLLSSVKEISDIVQR

The “isoahABL1(wt)” polypeptide can be utilized in a labeled peptide cleavage assay (for example, assay described in Example 17). The “isoblABL1(wt)” polypeptide can be utilized in a labeled peptide competition assay, such as a bioluminescence resonance energy transfer (BRET) intracellular assay (for example, Machleidt et al., ACS Chem. Biol. 10:1797-1804 (2015)), for example.

An ABL1 polypeptide referred to herein as an “ABL1 variant polypeptide” typically includes one or more amino acid substitutions relative to an ABL1(wt) polypeptide. An ABL1 variant polypeptide may include a structure described herein for an ABL1(wt) polypeptide, with the exception that the variant polypeptide includes one or more amino acid substitutions relative to the ABL1(wt) polypeptide. An ABL1 variant polypeptide sometimes includes up to ten amino acid substitutions relative to an ABL1(wt) polypeptide (for example, substitution of 1, 2, 3, 4, 5, 6, 7, 8, 9 or 10 amino acids relative to an ABL1(wt) polypeptide).

An amino acid substitution in an ABL1 variant polypeptide is defined herein relative to a position in SEQ ID NO:1, by the following notation utilizing the one-letter amino acid code: amino acid in SEQ ID NO:1—at position in SEQ ID NO:1—corresponding substituted amino acid in ABL1 variant polypeptide.

For example, the amino acid substitution notated as “T315I” refers to the threonine at position 315 in SEQ ID NO:1 substituted by isoleucine in an ABL1 variant (“ABL1(T315I)”). An ABL1 variant polypeptide can include an amino acid substitution corresponding to a position in SEQ ID NO:1 in instances where the ABL1 variant polypeptide includes the same number, or does not include the same number, of amino acids of the polypeptide of SEQ ID NO:1. A corresponding amino acid position of an amino acid substitution can be readily determined for a particular ABL1 variant polypeptide as known in the art. A corresponding amino acid position in an ABL1 variant polypeptide can be determined by aligning the ABL1 variant polypeptide to the polypeptide of SEQ ID NQ:1, as known in the art, and determining the position of the substituted amino acid in the ABL1 variant polypeptide corresponding to the amino acid position of SEQ ID NO:1 in the alignment (for example, World Wide Web Uniform Resource Locator (URL) Hypertext Transfer Protocol Secure: blast.ncbi.nlm.nih.gov/Blast.cgi?PAGE=Proteins). For example, the threonine highlighted in bold text and underlined in the catalytic domain polypeptide shown herein (SEQ ID NO:3) corresponds to threonine 315 in SEQ ID NO:1.

An ABL1 variant polypeptide often includes no amino acid insertion or amino acid deletion relative to the polypeptide of SEQ ID NO:1, SEQ ID NO:2 or SEQ ID NO:3. An ABL1 variant polypeptide can, in certain instances, include (i) an amino acid insertion within the polypeptide of SEQ ID NO:1, SEQ ID NO:2 or SEQ ID NO:3 containing 1, 2 or 3 contiguous amino acids; or (ii) an amino acid deletion of 1, 2 or 3 contiguous amino acids in the polypeptide of SEQ ID NO:1, SEQ ID NO:2 or SEQ ID NO:3; or a combination of (i) and (ii).

An ABL1 variant polypeptide may include an amino acid substitution in a particular portion of the polypeptide, non-limiting examples of which include a p-loop portion, SH3 contact portion, SH2 contact portion and A-loop portion. Non-limiting examples of amino acid substitutions, any one or more of which may be present in an ABL1 variant polypeptide, are illustrated in FIG. 2 (Soverini et al., Blood 118(5):1208-1215 (2011)). An ABL1 variant may include one or more of the following amino acid substitutions: M237V, I242T, M244V, K247R, L248V, G250E, G250R, Q252R, Q252H, Y253F, Y253H, E255K, E255V, E258D, W261 L, L273M, E275K, E275Q, D276G, T277A, E279K, V280A, V289A, V289I, E292V, E292Q, I293V, L298V, V299L, F311 L, F311I, T315A, T315I, F317L, F317V, F317I, F317C, Y320C, L324Q, Y342H, M343T, A344V, A350V, M351T, E355D, E355G, E355A, F359V, F359I, F359C, F359L, D363Y, L364I, A365V, A366G, L370P, V371A, E373K, V379I, A380T, F382L, L384M, L387M, L387F, L387V, M388L, Y393C, H396P, H396R, H396A, A397P, S417F, S417Y, I418S, I418V, A433T, S438C, E450K, E450G, E450A, E450V, E453G, E453K, E453V, E453Q, E459K, E459V, E459G, E459Q, M472I, P480L, F486S and E507G. The foregoing amino acid substitutions have been reported to confer imatinib therapy resistance in CML patients. Substitutions F317L and V299L have been reported to impart dasatinib therapy resistance and substitutions Y253H, E255K, E255V, F359V and F359C have been reported to impart nilotinib therapy resistance. The substitution T315I has been reported to impart resistance to imatinib, dasatinib and nilotinib therapy.

Non-limiting examples of ABL1 variant polypeptides are referred to herein as ABL1(M237V), ABL1(I242T), ABL1(M244V), ABL1(K247R), ABL1(L248V), ABL1(G250E), ABL1(G250R), ABL1(Q252R), ABL1(Q252H), ABL1(Y253F), ABL1(Y253H), ABL1(E255K), ABL1(E255V), ABL1(E258D), ABL1(W261 L), ABL1(L273M), ABL1(E275K), ABL1(E275Q), ABL1(D276G), ABL1(T277A), ABL1(E279K), ABL1(V280A), ABL1(V289A), ABL1(V289I), ABL1(E292V), ABL1(E292Q), ABL1(I293V), ABL1(L298V), ABL1(V299L), ABL1(F311 L), ABL1(F311I), ABL1(T315A), ABL1(T315I), ABL1(F317L), ABL1(F317V), ABL1(F317I), ABL1(F317C), ABL1(Y320C), ABL1(L324Q), ABL1(Y342H), ABL1(M343T), ABL1(A344V), ABL1(A350V), ABL1(M351T), ABL1(E355D), ABL1(E355G), ABL1(E355A), ABL1(F359V), ABL1(F359I), ABL1(F359C), ABL1(F359L), ABL1(D363Y), ABL1(L364I), ABL1(A365V), ABL1(A366G), ABL1(L370P), ABL1(V371A), ABL1(E373K), ABL1(V379I), ABL1(A380T), ABL1(F382L), ABL1(L384M), ABL1(L387M), ABL1(L387F), ABL1(L387V), ABL1(M388L), ABL1(Y393C), ABL1(H396P), ABL1(H396R), ABL1(H396A), ABL1(A397P), ABL1(S417F), ABL1(S417Y), ABL1(I418S), ABL1(I418V), ABL1(A433T), ABL1(S438C), ABL1(E450K), ABL1(E450G), ABL1(E450A), ABL1(E450V), ABL1(E453G), ABL1(E453K), ABL1(E453V), ABL1(E453Q), ABL1(E459K), ABL1(E459V), ABL1(E459G), ABL1(E459Q), ABL1(M472I), ABL1(P480L), ABL1(F486S) or ABL1(E507G).

A non-limiting example of an ABL1 variant polypeptide contains the polypeptide
of SEQ ID NO: 3 with one or more of the following modifications: (i) the
threonine highlighted in bold text and underlined in the polypeptide of SEQ ID
NO: 3 herein is substituted to isoleucine, (ii) a polyhistidine tag containing
ten consecutive histidine amino acids is appended at the N-terminus along with
an adjacent 3′ linker sequence (SSGVDLGT) followed by a “TEV” cleavage site
(ENLYFQ/S), and (iii)a n initial “MG” sequence. An example of an ABL1
variant polypeptide containing the foregoing modifications is referred to as
“catABL1(T3151)” herein and contains the following polypeptide (SEQ ID NO: 6).
MGHHHHHHHHHHSSGVDLGTENLYFQ/SSPNYDKWEMERTDITMKHKLGGGQYGEVYEGVWKKY
SLIVAVKTLKEDTMEVEEFLKEAAVMKEIKHPNLVQLLGVCTREPPFYIIIEFMTYGNLLDYLR
ECNRQEVNAVVLLYMATQISSAMEYLEKKNFIHRDLAARNCLVGENHLVKVADFGLSRLMTGDT
YTAHAGAKFPIKWTAPESLAYNKFSIKSDVWAFGVLLWEIATYGMSPYPGIDLSQVYELLEKDY
RMERPEGCPEKVYELMRACWQWNPSDRPSFAEIHQAFETMFQESSISDEVEKELGK

In the polypeptide of SEQ ID NO:6 depicted above, the forward slash designates the TEV cleavage site and does not designate an amino acid (i.e., the Q and S immediately flanking the forward slash are contiguous).

An ABL1 variant polypeptide can have the same or about the same substrate phosphorylation activity of an ABL1(wt) polypeptide containing the polypeptide of SEQ ID NO:1 under phosphorylation conditions, when the polypeptide is not contacted by a test compound. An ABL1 variant polypeptide can have a substrate phosphorylation activity lower than the phosphorylation activity of an ABL1(wt) polypeptide containing the polypeptide of SEQ ID NO:1 under phosphorylation conditions, when the polypeptide is not contacted by a test compound (for example, a substrate phosphorylation activity within about 10-fold, or 9-fold, or 8-fold, or 7-fold, or 6-fold, or 5-fold, or 4-fold, or 3-fold, or 2-fold, lower than the substrate phosphorylation activity of the ABL (wt) polypeptide). An ABL1 variant polypeptide can have a substrate phosphorylation activity greater than the phosphorylation activity of an ABL1(wt) polypeptide containing the polypeptide of SEQ ID NO:1 under phosphorylation conditions, when the polypeptide is not contacted by a test compound (for example, a substrate phosphorylation activity within about 10-fold, or 9-fold, or 8-fold, or 7-fold, or 6-fold, or 5-fold, or 4-fold, or 3-fold, or 2-fold, greater than the substrate phosphorylation activity of the ABL1(wt) polypeptide). Phosphorylation activity of an ABL1 variant polypeptide can be determined by a suitable assay, which can be an in vitro assay (for example, a labeled peptide cleavage assay described herein), for example.

### Protein Kinase Inhibitor Assessment

A compound herein can inhibit a protein kinase (PK) activity. A compound can be assessed as a test compound in a suitable assay or system to determine PK inhibitor activity. A test compound assessed by an assay for PK inhibitor activity sometimes is a compound herein and/or sometimes is another compound, such as a clinically approved PK inhibitor, for example. An assay sometimes is conducted in vitro or in vivo.

An in vitro assay sometimes quantifies substrate phosphorylation activity catalyzed by a PK polypeptide under phosphorylation conditions in the presence or absence of a test compound that can bind to the PK and potentially inhibit the PK phosphorylation activity. A suitable substrate can be utilized in an assay, such as a polypeptide or peptide substrate, for example. A peptide substrate for assessing ABL1 inhibitor activity can contain the amino acid sequence EAIYAAPFAKKK (SEQ ID NO:7), for example. An assay for quantifying substrate phosphorylation activity inhibition sometimes outputs one or more of an IC₅₀ value, Kᵢ value, K_{d} value, K_{off} value and Kₒₙ value as a quantification of PK inhibition and/or binding.

A non-limiting example of an assay for quantifying substrate phosphorylation activity inhibition analyzes a peptide substrate capable of being phosphorylated by a PK polypeptide, referred to herein as an “PK peptide substrate.” A PK peptide substrate sometimes is labeled with one or more detectable labels (for example, a fluorescent agent). Fluorescence from a PK peptide substrate labeled with a fluorescent agent sometimes is assessed in an assay. In certain embodiments, a PK peptide substrate end-labelled with a distinct donor fluorophore on one end and a distinct acceptor fluorophore on the other end is utilized. In certain instances, the donor and the acceptor fluorophores are a Fluorescence Resonance Energy Transfer (FRET) pair. In certain embodiments, the donor fluorophore is coumarin and the acceptor fluorophore is fluorescein. In certain instances, a PK peptide substrate is cleaved in an assay after the peptide is exposed to phosphorylation conditions. An assay in which a PK peptide substrate is labeled with one or more detection agents and exposed to cleavage conditions is referred to as a “labeled peptide cleavage assay” and “peptide cleavage assay.”

In certain embodiments, a labeled peptide cleavage assay includes: (1) contacting an unphosphorylated peptide, labeled at each end with a FRET pair fluorophore, with a test compound and a PK polypeptide having a PK phosphorylation activity, under phosphorylation conditions, thereby generating a phosphorylated peptide; (2) exposing the peptide, after (1), to phosphorylation state-dependent cleavage conditions, thereby generating cleaved peptide; and (3) measuring and analyzing, after (2) a fluorescent emission signal from one or both fluorophores. A non-limiting example of a labeled peptide cleavage assay is described in Example 17 (Z'LYTE™ assay).

Under phosphorylation conditions, a PK polypeptide typically is capable of transferring a phosphate from a provided cofactor (for example, adenosine triphosphate (ATP)) to the substrate (for example, peptide or protein substrate), where presence of a PK inhibitor compound reduces the amount of substrate phosphorylation compared to conditions in which the compound is not present. Under phosphorylation state-dependent cleavage conditions, (i) the phosphorylated substrate, but not the unphosphorylated substrate, is capable of being cleaved, or (ii) the phosphorylated substrate is preferentially cleaved relative to the unphosphorylated substrate, or (iii) the unphosphorylated substrate, but not the phosphorylated substrate, is capable of being cleaved, or (iv) the unphosphorylated substrate is preferentially cleaved relative to the phosphorylated substrate. The substrate typically is exposed to cleavage conditions after the substrate is contacted with a PK polypeptide under phosphorylation conditions. The substrate can be cleaved by a peptidase or protease enzyme under cleavage conditions.

After the substrate is exposed to phosphorylation conditions and cleavage conditions, a fluorescent signal from cleaved substrate can be measured. In certain embodiments for a substrate containing a donor and acceptor FRET pair, a ratio of donor emission to acceptor emission (or a ratio of acceptor emission to donor emission) after excitation of the donor can be determined to assess the degree of peptide cleavage and thereby degree of peptide phosphorylation. An IC₅₀ value can be determined from such a ratio as known in the art.

Another type of assay involving FRET is a tracer displacement assay, a non-limiting example of which is described in Example 17 (LanthaScreen™ Eu Kinase Binding Assay). A tracer binding assay measures test compound binding to a PK. In a tracer displacement assay, a PK is contacted with (i) a tracer ligand, labeled with a FRET pair fluorophore, that binds to the PK under binding conditions; (ii) a test molecule that displaces the tracer ligand when the test molecule binds to the PK under the binding conditions; and (iii) an antibody, conjugated to another FRET pair fluorophore, that binds to the PK. Binding of the antibody and tracer ligand to the PK results in a FRET signal, and displacement of tracer ligand from the PK by a test molecule that competes with the tracer ligand for binding to the PK results in a reduction of the FRET signal.

Another type of assay that can be utilized to quantify phosphorylation activity inhibition is an adenosine diphosphate (ADP) formation assay. The assay can be used to quantify ATP hydrolysis, including the intrinsic ATPase activity of a PK that transfers a terminal phosphate from ATP to water (and not a peptide substrate), thereby generating ADP from ATP. A non-limiting example of an ADP formation assay is described in Kashem et al., J. Biomol. Screen. 12:70-83 (2007) and is described in Example 17 (Adapta™ Assay).

Another type of assay that can be utilized to quantify binding of a test compound to a PK is a solid phase inhibitor competition assay. The assay can be used to quantify binding of a test compound to a PK tagged with a nucleic acid detection tag in competition with a solid phase-associated PK inhibitor. The amount of the PK associated with the solid phase, assessed by PCR quantification of the detection tag, determines the level of PK binding to the test compound. A non-limiting example of a solid phase inhibitor competition assay is a KINOMEscan™ assay (EuroFins Discovery, described in Fabian et al., Nat. Biotechnol. 23: 329-336 (2005) for example).

### Uses of Compounds

A compound can be used in a variety of applications. A compound can be used to (i) inhibit one or more PKs in vitro, ex vivo, or in vivo, (ii) inhibit one or more PKs in cells, organs and/or tissues administered a composition containing a compound; and/or (iii) inhibit one or more PKs in a subject administered a composition containing the compound. A compound can be utilized in studies, including, for example: (i) studies of PK inhibitors (for example, in vitro assay studies); (ii) pre-clinical in vivo animal studies, including xenograft studies in mice and pharmacokinetic studies in higher animals (for example, rats, dogs and/or monkeys); and (iii) human clinical studies. A compound herein can be utilized as a reference compound in a study of other compounds (for example, negative control or positive control), such as in an in vitro assay study, pre-clinical study and/or clinical study, for example.

A compound herein can be (i) for treatment of a medical condition; (ii) prepared as a composition (for example, pharmaceutical composition) or medicament for treatment of a medical condition; and/or (iii) utilized in a method for treating a medical condition in which a compound is administered to a subject in need thereof in an amount sufficient to treat the medical condition (an effective amount). For embodiments in which a subject is treated, the subject can be human (Homo sapiens) and can be an adult or pediatric patient. A medical condition sometimes is a PK-associated condition, such as a medical condition associated with a PK aberration and/or dysregulation of a PK (for example, dysregulation of a PK gene). A PK aberration can be a PK modification, which, for example, can be a nucleic acid translocation or other modification associated with a PK gene A PK modification can be outside of a PK gene coding region and result in dysregulation of the PK gene. A PK modification can be an addition, deletion or substitution of one or more amino acids in the PK encoded by a PK gene. A PK translocation can be within a PK gene coding region and result in a PK gene truncation and/or translocation of a PK gene or portion thereof to a different chromosome or different location of the same chromosome. A PK translocation can result in a fusion of a PK gene or portion thereof with another nucleic acid portion from a different chromosome or different location of the same chromosome. A PK aberration can result in an altered PK activity relative to the PK activity when the aberration is not present. An altered activity can be (i) altered binding affinity to an inhibitor, (ii) altered binding activity to a native binding partner, and/or (iii) increased phosphorylation activity due to PK overexpression or increased intrinsic activity, for example. A PK-associated medical condition can be caused by an aberration of (for example, dysregulation and/or modification of) one or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

A medical condition sometimes is a cell proliferative condition such as a cancer for example, and a compound can be used to treat a cancer condition. A compound can be used to treat a cancer condition associated with one or more PK modifications, including modification of one or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK. Non-limiting examples of cancers include lymphomas, thymomas, leukemias, carcinomas, gliomas, sarcomas (including liposarcoma), adenocarcinomas, adenosarcomas, and adenomas. Non-limiting examples of cancers are cancers occurring in lymph nodes, blood, thymus, breast, heart, lung, small intestine, colon, rectum, spleen, kidney, bladder, head, neck, esophagus, ovary, prostate, brain, pancreas, skin, bone, bone marrow, uterus, testicles, cervix and liver.

Cancers include leukemia or lymphoid malignancies, hematologic malignancies, such as Hodgkin's lymphoma; non-Hodgkin's lymphomas, including Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia; tumors of lymphocyte precursor cells, including B-cell acute lymphoblastic leukemia/lymphoma and T-cell acute lymphoblastic leukemia/lymphoma; thymoma; tumors of the mature T and NK cells, including peripheral T-cell leukemias, adult T-cell leukemia/T-cell lymphomas and large granular lymphocytic leukemia; Langerhans cell histocytosis; a myeloid neoplasia including acute myelogenous leukemia (AML), AML with maturation, AML without differentiation, acute promyelocytic leukemia, acute myelomonocytic leukemia, and acute monocytic leukemias; myelodysplastic syndromes; and chronic myeloproliferative disorders, including chronic myelogenous leukemia.

Cancers include colorectal and head and neck tumors; squamous cell carcinoma of the head and neck; brain tumors such as glioblastomas; tumors of the lung, breast, pancreas, esophagus, bladder, kidney, ovary, cervix, and prostate; central nervous system neoplasms; neuroblastomas; capillary hemangioblastomas; meningiomas and cerebral metastases; melanoma; gastrointestinal and renal carcinomas and sarcomas; rhabdomyosarcoma; glioblastoma, including glioblastoma multiforme; leiomyosarcoma; lymphoma; blastoma; neuroendocrine tumors; mesothelioma; schwannoma; meningioma; tumors of the central nervous system, including glioma, glioblastoma, neuroblastoma, astrocytoma, medulloblastoma, ependymoma, and retinoblastoma; solid tumors of the head and neck, including nasopharyngeal cancer, salivary gland carcinoma, and esophageal cancer; a lung cancer including small-cell lung cancer, non-small cell lung cancer (NSCLC), adenocarcinoma of the lung and squamous carcinoma of the lung; a digestive system cancer, gastric cancer or stomach cancer, including gastrointestinal cancer, cancer of the bile duct or biliary tract, colon cancer, colon adenocarcinoma, rectal cancer, colorectal cancer, and anal carcinoma; a reproductive system cancer including testicular, penile, or prostate cancer, uterine, vaginal, vulval, cervical, ovarian, and endometrial cancer; thyroid cancer, including medullary thyroid cancer (MTC), thyroid gland medullary carcinoma, papillary thyroid cancer (PTC); skin cancer, including melanoma, cutaneous carcinoma, basal cell carcinoma, squamous cell cancer, actinic keratosis; liver cancer, including hepatic carcinoma, hepatocellular cancer, and hepatoma; bone cancer, including osteoclastoma, and osteolytic bone cancers; cancer of additional tissues and organs, including pancreatic cancer, bladder cancer, kidney or renal cancer, breast cancer, cancer of the peritoneum, and Kaposi's sarcoma; tumors of the vascular system, including angiosarcoma and hemangiopericytoma; and blood cancers including leukemia, myelodysplastic syndrome (MDS), myelofibrosis, polycythemia vera and essential thrombocythemia. A cancer can be treated with a composition containing a compound herein as an active ingredient. In certain embodiments a composition used to treat a cancer contains a compound of Subgroup 1 or Subgroup 5.

Particular blood cancers include leukemias, and leukemias include chronic myeloid leukemia (CML) and acute lymphoblastic leukemia (ALL). A CML that can be treated includes chronic phase CML (CML-CP), acute phase CML (CML-AP) and blast phase CML (CML-BP). An ALL that can be treated includes a relapsed and/or refractory ALL (R/R ALL), Philadelphia chromosome-positive ALL, and other forms of ALL described herein (for example, Philadelphia chromosome-positive-like-ALL, B-ALL, T-ALL).

A leukemia sometimes is associated with an ABL family PK aberration, and sometimes an ABL1 PK aberration. A compound that inhibits an ABL family PK (for example, an ABL1 PK) can be used to treat a leukemia (for example, CML and/or an ALL such as Philadelphia chromosome-positive ALL). A compound that effectively inhibits or moderately inhibits ABL1, ABL1-T315I, BTK, AURKA, JAK2 and JAK3, and optionally JAK1, can be used to treat a leukemia (for example, a CML and/or an ALL such as Philadelphia chromosome-positive ALL). A compound that effectively inhibits or moderately inhibits ABL1, ABL1-T315I, BTK, AURKA, JAK2 and JAK3, and optionally mildly inhibits or ineffectively inhibits JAK1, can be used to treat a leukemia (for example, a CML and/or an ALL). A compound that effectively inhibits or moderately inhibits one or more of all of TYK2, a TRK family PK, ROS1, TXK, an EPH family PK, IRAK3, PLK4, TNK1, RET and JAK2-V617F can be used to treat a leukemia (for example, a CML and/or an ALL such as Philadelphia chromosome-positive ALL).

A leukemia, such as CML and/or an ALL for example, can be treated with a compound of Subgroup 1 or Subgroup 5. For example, a Philadelphia chromosome-positive ALL and/or R/R Philadelphia chromosome-positive ALL, can be treated with a compound of Subgroup 1 or Subgroup 5.

A leukemia can be a Philadelphia chromosome-positive-like ALL, which has been associated with a rearrangement involving ABL1, ABL2, CRLF2, CSF1R, EPOR, JAK2, NTRK3, PDGFRB, PTK2B, TSLP and/or TYK2 and/or a sequence mutations involving FLT3, IL7R and/or SH2B3 (see, for example, Roberts et al., N Engl J Med 371(11): 1005-1015 (2014)). A compound that effectively inhibits or moderately inhibits ABL2, TYK2, TRKC and PTK2B, can be used to treat a Philadelphia chromosome-positive-like ALL. A compound that effectively inhibits or moderately inhibits ABL1, ABL1-T315I, BTK, AURKA, JAK2 and JAK3, and optionally JAK1, can be used to treat a Philadelphia chromosome-positive-like ALL. A compound that effectively inhibits or moderately inhibits ABL1, ABL1-T315I, BTK, AURKA, JAK2 and JAK3, and optionally mildly inhibits or ineffectively inhibits JAK1, can be used to treat a Philadelphia chromosome-positive-like ALL. A compound that effectively inhibits or moderately inhibits one or more of all of TYK2, a TRK family PK, ROS1, TXK, an EPH family PK, IRAK3, PLK4, TNK1, RET and JAK2-V617F can be used to treat a Philadelphia chromosome-positive-like ALL. A Philadelphia chromosome-positive-like ALL can be treated with a compound of Subgroup 1 or Subgroup 5.

A leukemia can be a B-cell acute lymphoblastic leukemia (B-ALL). A B-ALL can be a TCF3-HLF-positive B-ALL, which can be a TCF3-HLF-positive acute B-ALL. A TCF3-HLF-positive B-ALL typically is a B-ALL harboring a t(17;19)(q22;p13) translocation, producing an aberrant TCF3-HLF fusion (see, for example, Leonard et al., Haematologica 106(11): 2990-2994 (2021)). A TCF3-HLF-positive B-ALL can be associated with an AURKA aberration, and a compound that effectively inhibits or moderately inhibits an AURKA PK can be used to treat a B-ALL, such as a TCF3-HLF-positive B-ALL for example. A compound that effectively inhibits or moderately inhibits ABL1, ABL1-T315I, BTK, AURKA, JAK2 and JAK3, and optionally JAK1, can be used to treat a B-ALL, such as a TCF3-HLF-positive B-ALL for example. A compound that effectively inhibits or moderately inhibits ABL1, ABL1-T315I, BTK, AURKA, JAK2 and JAK3, and optionally mildly inhibits or ineffectively inhibits JAK1, can be used to treat a B-ALL, such as a TCF3-HLF-positive B-ALL for example. A compound that effectively inhibits or moderately inhibits ABL1, ABL1-T315I, BTK, AURKA, JAK2, NTRK3 (for example, a ETV6-NTRK3 fusion), PTK2B, TYK2 and ABL2 can be used to treat a B-ALL, such as a TCF3-HLF-positive B-ALL for example, where the compound optionally can effectively inhibit or moderately inhibit JAK1 or the compound optionally can mildly inhibit or ineffectively inhibit JAK1. A compound that effectively inhibits or moderately inhibits one or more of all of TYK2, a TRK family PK, ROS1, TXK, an EPH family PK, IRAK3, PLK4, TNK1, RET and JAK2-V617F can be used to treat a B-ALL, such as a TCF3-HLF-positive B-ALL for example. A B-ALL, including a TCF3-HLF-positive B-ALL for example, can be treated with a compound of Subgroup 1 or Subgroup 5.

A subject identified as having an ABL1-T315I variant can be treated with a compound herein (for example, for treatment of a leukemia such as a CML and/or an ALL such as Philadelphia chromosome-positive ALL). A subject not identified as having an ABL1-T315I variant or identified as not having an ABL1-T315I variant, and is resistant and/or intolerant to at least two PK inhibitors, can treated with a compound herein (for example, for treatment of a leukemia such as a CML and/or ALL such as Philadelphia chromosome-positive ALL). A subject having no available protein kinase inhibitor options can be administered a compound herein to treat CML.

A leukemia can be a T-cell acute lymphoblastic leukemia (T-ALL), which can be a R/R T-ALL. A T-ALL can be associated with a LCK family PK aberration, and a compound that effectively inhibits or moderately inhibits a LCK family PK (for example, LCK(wt)) can be used to treat a T-ALL, including R/R T-ALL. A compound that effectively inhibits or moderately inhibits ABL1, ABL1-T315I, BTK, AURKA, JAK2 and JAK3, and optionally JAK1, can be used to treat a T-ALL, including R/R T-ALL for example. A compound that effectively inhibits or moderately inhibits ABL1, ABL1-T315I, BTK, AURKA, JAK2 and JAK3, and optionally mildly inhibits or ineffectively inhibits JAK1, can be used to treat a T-ALL, including R/R T-ALL for example. A compound that effectively inhibits or moderately inhibits one or more of all of TYK2, a TRK family PK, ROS1, TXK, an EPH family PK, IRAK3, PLK4, TNK1, RET and JAK2-V617F can be used to treat a T-ALL, including R/R T-ALL for example. A T-ALL, including R/R T-ALL for example, can be treated with a compound of Subgroup 1 or Subgroup 5.

A cancer can be a solid cancer, such as a colon cancer, lung cancer (for example, a lung carcinoma), breast cancer, blood cancer or ovarian cancer. A solid cancer can be associated with a LCK family PK aberration, and a compound that effectively inhibits or moderately inhibits a LCK family PK (for example, LCK(wt)) can be used to treat a solid cancer. Non-limiting examples of solid tumor blood cancers include peripheral T-cell lymphoma (PTCL), PTCL-NOS (not otherwise specified) and PTCL and PTCL-NOS associated with a KHDRBS1-LCK gene fusion (for example, Debackere et al., Nat Comm 12:3705, World Wide Web address URL doi.org/10.1038/s41467-021-24037-4 (2021)). A compound that effectively inhibits or moderately inhibits a LCK family PK can also can be used to treat atherosclerotic coronary vascular disease (ASCVD). A compound that effectively inhibits or moderately inhibits ABL1, ABL1-T315I, BTK, AURKA, JAK2 and JAK3, and optionally JAK1, can be used to treat a solid cancer or ASCVD. A compound that effectively inhibits or moderately inhibits ABL1, ABL1-T315I, BTK, AURKA, JAK2 and JAK3, and optionally mildly inhibits or ineffectively inhibits JAK1, can be used to treat a solid cancer or ASCVD. A compound that effectively inhibits or moderately inhibits one or more of all of TYK2, a TRK family PK, ROS1, TXK, an EPH family PK, IRAK3, PLK4, TNK1, RET and JAK2-V617F can be used to treat a solid cancer or ASCVD. A solid cancer or ASCVD can be treated with a compound of Subgroup 1 or Subgroup 5.

A cancer can be a lung cancer, such as non-small cell lung cancer (NSCLC). In certain instances, the lung cancer is associated with a DDR family PK aberration and/or a SRC family PK aberration. In certain instances, the DDR family PK aberration is a DDR2 variant PK aberration. The DDR2 variant PK contains a T654M and/or N456S amino acid substitution in certain instances. A compound that inhibits a SRC family PK or DDR family PK is used to treat a lung cancer (for example NSCLC) in certain embodiments. In certain embodiments, a compound that effectively inhibits or moderately inhibits SRC(wt), SCR—N1, DDR2(wt) and/or a DDR2 variant containing a T654M and/or N456S amino acid substitution is used to treat a lung cancer, such as NSCLC for example. A compound that effectively inhibits or moderately inhibits ABL1, ABL1-T315I, BTK, AURKA, JAK2 and JAK3, and optionally JAK1, can be used to treat a lung cancer (for example NSCLC) in certain embodiments. A compound that effectively inhibits or moderately inhibits one or more or all of ABL1, ABL1-T315I, BTK, AURKA, JAK2 and JAK3, and optionally mildly inhibits or ineffectively inhibits JAK1, can be used to treat a lung cancer (for example NSCLC). A compound that effectively inhibits or moderately inhibits one or more of all of TYK2, a TRK family PK, ROS1, TXK, an EPH family PK, IRAK3, PLK4, TNK1, RET and JAK2-V617F can be used to treat a lung cancer (for example NSCLC). A lung cancer, such as NSCLC for example, can be treated with a compound of Subgroup 1 or Subgroup 5.

A compound that inhibits a TRK family PK can be utilized to treat a cancer associated with a TRK family PK aberration. A cancer sometime is a cancer positive for a TRK family PK fusion. In certain instances, the cancer is a solid tumor positive for a TRK family PK fusion, and optionally where the TRK portion of the fusion does not contain a known acquired resistance amino acid substitution.

A compound that inhibits a ROS family PK can be utilized to treat a cancer associated with a ROS family PK aberration. In certain instances, the cancer is a ROS1 positive lung cancer, including without limitation a solid tumor and/or a lung cancer. A ROS1 positive lung cancer can be a lung nodule cancer, non-small cell lung cancer, small cell lung cancer or mesothelioma.

A compound that inhibits an EPH family PK can be utilized to treat a cancer associated with an EPH family PK aberration. In certain instances, the cancer is associated with an EPHA1 aberration, EPHA2 aberration and/or an EPHB1 aberration, non-limiting examples of which include a breast cancer, lung cancer, brain cancer, spinal cancer, gastric cancer, or skin cancer, and optionally a solid tumor cancer. In certain instances, the lung cancer is non-small cell lung cancer (NSCLC); the skin cancer is myeloma; and the brain cancer or spinal cancer is a glioblastoma.

A compound that inhibits a TNK family PK can be utilized to treat a cancer associated with a TNK family PK aberration. In certain instances, a cancer is associated with a TNK1 PK aberration, such as a cancer deficient in LKB1, for example. In certain instances, a cancer associated with a TNK1 aberration is a cancer associated with a TNK1 variant to which binding of a 14-3-3 protein is weaker than to TNK1(wt), a non-limiting example of which is Hodgkin lymphoma, for which the Hodgkin lymphoma cell line L540 can be representative.

A compound that inhibits a RET family PK can be utilized to treat a cancer associated with a RET family PK aberration (for example, a cancer associated with a RET family PK fusion). In certain instances, a cancer associated with a RET family PK is lung cancer, including non-small cell lung cancer (NSCLC) and/or lung adenocarcinoma; a thyroid cancer, including medullary thyroid cancer (MTC), thyroid gland medullary carcinoma and/or papillary thyroid cancer (PTC); a colon cancer, including colon adenocarcinoma; and/or a skin cancer including melanoma and/or cutaneous melanoma.

A compound that inhibits a PLK4 family PK can be utilized to treat a cancer associated with a PLK family PK aberration. In certain instances, a cancer is associated with a PLK4 aberration, non-limiting examples of which include liver cancer, breast cancer and AML. A compound that inhibits an IRAK family PK can be utilized to treat a cancer associated with an IRAK family PK aberration. In certain instances a cancer is associated with an IRAK3 aberration. In certain instances, a compound of Subgroup 9 is used to treat a cancer associated with a PLK4 aberration or an IRAK3 aberration. In certain instances, a compound herein used to treat a cancer associated with an IRAK3 aberration is used in combination with an immune checkpoint blockade (ICB) therapeutic, examples of which are known.

A compound that inhibits a JAK family PK can be utilized to treat a medical condition associated with a JAK family PK aberration (a JAK-associated medical condition). A JAK-associated medical condition sometimes is a JAK2-associated medical condition, and compound that selectively inhibits a JAK2 PK is used to treat the medical condition. In certain instances, a compound of Subgroup 2, Subgroup 3, Subgroup 4, Subgroup 6, Subgroup 7 or Subgroup 8 is utilized to treat a JAK2-associated medical condition.

In certain embodiments, a JAK2-associated medical condition is a cancer, including without limitation, a lung cancer, breast cancer, head cancer or neck cancer. In certain embodiments, a JAK2-associated medical condition is a blood cancer, including without limitation, myelodysplastic syndrome (MDS), myelofibrosis, polycythemia vera or essential thrombocythemia. In certain instances, a JAK2-associated cancer is positive for a JAK2 variant containing a V617F substitution. In certain instances, a compound used to treat a cancer positive for a JAK2 variant containing a V617F substitution is of Subgroup 3 or Subgroup 7.

A compound can be used to treat minimum residual disease (MRD) associated with a cancer condition, and can be a compound that inhibits a JAK family PK. A compound that is an effective inhibitor, or optionally a moderate inhibitor, of a JAK PK can be used to treat a cancer condition with potentially higher efficacy, as compared to treatment of the condition with a compound that is not an effective inhibitor or not a moderate inhibitor of a JAK PK. A compound that is an effective inhibitor of, or moderate inhibitor of, a JAK family PK, and an effective inhibitor of or moderate inhibitor of one or more other family PKs, can be used to treat, with potentially high efficacy, a cancer associated with the one or more other family PKs. For example, a compound that is an effective inhibitor of a JAK family PK and of an ABL family PK can be used to treat an ABL1-associated blood cancer, including CML and Philadelphia chromosome-positive ALL, for example, with potentially higher efficacy than a compound that effectively inhibits the ABL family PK but not the JAK family PK.

In certain embodiments, a compound is used to treat an inflammation condition, autoimmune condition and/or skin condition. In certain instances, the inflammation condition is a chronic inflammation condition and/or senescent cell chronic inflammation condition such as a senescence-associated secretory phenotype (SASP) condition, for example. In certain instances, the autoimmune condition is atopic dermatitis, non-segmental vitiligo or rheumatoid arthritis. In certain instances, the rheumatoid arthritis is intolerant to one or more tumor necrosis factor (TNF) blockers. In certain instances, the skin condition is atopic dermatitis, non-segmental vitiligo, psoriasis (for example, plaque psoriasis), ultraviolet (UV) damaged skin, severely UV damaged skin or aged skin. A topical cream containing a compound can be used to treat a inflammation condition, autoimmune condition or skin condition. A compound can reduce an amount of a cytokine associated with an inflammation condition, autoimmune condition and/or skin condition, such as interleukin-6 (IL-6) for example, sometimes with subnanomolar efficacy.

In certain embodiments, a compound is used to treat a medical condition associated with a TYK family PK aberration, such as a medical condition associated with a TYK2 PK aberration. In certain embodiments, a compound is used to treat psoriasis, which can be moderate to severe psoriasis. In certain embodiments, a compound is used to treat plaque psoriasis, which can be moderate to severe plaque psoriasis. In certain instances, a compound is used to treat a subject who is a candidate for systemic therapy or phototherapy. In certain embodiments, the compound inhibits a TYK family PK, and in certain instances, the compound inhibits TYK2(wt). In certain embodiments, the compound is a Subgroup 10 compound.

In certain embodiments, an inflammation condition, autoimmune condition and/or skin condition is treated with a compound that effectively inhibits and selectively inhibits a JAK2 PK. An example of a compound that effectively inhibits and selective inhibits a JAK2 PK is a compound that effectively inhibits JAK2(wt) PK, does not effectively inhibit JAK (wt) PK, and optionally effectively inhibits or moderately inhibits JAK3(wt) PK. In certain instances, a skin condition such as atopic dermatitis or non-segmental vitiligo, for example, is treated with a compound of Subgroup 2 or Subgroup 3. In certain embodiments, an autoimmune condition such as moderate to severe rheumatoid arthritis (for example, rheumatoid arthritis intolerant to a TNF inhibitor), for example, is treated with a compound of Subgroup 6, Subgroup 7 or Subgroup 8.

A compound that is an effective inhibitor and selective inhibitor of a JAK2 PK can be used to treat a condition with a potentially lower incidence of a serious adverse event, as compared to treatment of the condition with a compound that is not a selective inhibitor of a JAK2 PK. Non-limiting examples of compounds that are not selective inhibitors of JAK2 PK are tofacitinib or ruxolitinib. A serious adverse event can be a malignancy, serious adverse cardiovascular event and/or blood clot, mortality or infection. In certain embodiments, a compound that is an effective inhibitor and selective inhibitor of a JAK2 PK can be utilized to treat a condition in a subject having a prior history of heart disease, and to whom a compound that is not a selective inhibitor of JAK2 would not be administered.

A moderate to severe form of a medical condition can be treated. For example, moderate to severe rheumatoid arthritis or moderate to severe plaque psoriasis can be treated. A stage Ill or stage IV cancer condition can be treated (for example, a stage IV ROS1 positive lung cancer), for example.

A medical condition associated with a particular variant PK can be treated. In certain instances, a blood cancer, such as myelofibrosis, MDS, polycythemia vera or essential thrombocytopenia, for example, can be treated in subjects from which a sample was assessed as having a JAK2 containing a V617F variation. Such subjects can be treated with a compound that effectively inhibits a JAK2 variant containing the V617F variation (a compound of Subgroup 3 or Subgroup 7, for example). In certain instances, a leukemia, such as CML or Philadelphia chromosome-positive ALL, for example, can be treated in subjects from which a sample was assessed as having an ABL1 variant containing a T315I substitution. Such subjects can be treated with a compound that effectively inhibits an ABL1 variant containing a T315I substitution, such as a compound of Subgroup 1 or Subgroup 5, for example.

In certain embodiments, it is determined whether a PK having a particular variation (a PK variant) is present or absent in a sample from a subject, and if the PK variant is identified as being present in the sample, then a particular medical condition associated with the PK variant is treated. In certain instances, presence or absence of a JAK2 variant containing a V617F substitution is screened in a sample from a subject, and if the variant is present in the sample, the subject may be treated for a particular medical condition associated with the variant (for example, a blood cancer such as myelofibrosis, MDS, polycythemia vera or essential thrombocytopenia, for example). In certain instances, presence or absence of an ABL1 variant containing a T315I substitution is screened in a sample from a subject, and if the variant is present in the sample, the subject may be treated for a particular medical condition associated with the variant (for example, a leukemia such as CML or Philadelphia chromosome-positive ALL, for example). In certain instances, presence or absence of a DDR2 variant containing N456S and/or T654M substitution is screened in a sample from a subject, and if the variant is present in the sample, the subject may be treated for a particular medical condition associated with the variant (for example, a lung cancer such as non-small cell lung cancer (NSCLC)). In certain instances, presence or absence of a RET variant containing one or more of A883F, G691S, M918T, S891A, V804E, V804L, V804M and Y791F is screened in a sample from a subject, and if the variant is present in the sample, the subject may be treated for a particular medical condition associated with the variant (for example, a lung cancer (NSCLC) or thyroid cancer (medullary thyroid cancer (MTC), papillary thyroid cancer (PTC) or thyroid gland medullary carcinoma, for example)).

A compound typically is utilized in an amount sufficient to inhibit a PK activity (an effective amount). An “effective amount” often is a dosage sufficient to affect a beneficial or desired result. Non-limiting examples of desired results include reducing a PK activity (e.g., test compound binding activity; substrate phosphorylation activity); decreasing, attenuating and/or stabilizing one or more symptoms associated with a condition; increasing quality of life of a subject suffering from a condition; decreasing the dose of other medications required to treat the condition; enhancing the effect of another medication; delaying the progression of the condition; and/or prolonging survival of a subject. Non-limiting examples of symptoms associated with cancer include presence and/or proliferation of cancer cells; presence and/or growth of one or more tumors; cancer metastases and the like. An effective amount can be an amount sufficient to: kill cancer cells; reduce the rate of cancer cell proliferation; and/or eliminate, reduce and/or delay metastasis from a primary site of cancer. An effective amount may be in conjunction with another therapeutic agent. An effective amount may be considered in the context of administering a compound described herein without another therapeutic agent or with another therapeutic agent. An optimal range of an effective amount of each component can be determined.

An effective amount can be determined by standard clinical techniques and can be a technique for determining dosage. An effective amount often depends on the route of administration and the seriousness of the condition, and often is decided according to the judgment of the practitioner and circumstances of each subject.

An effective amount can be administered in one or more administrations. Non-limiting types of administration include parenteral administration (e.g., intradermal, intramuscular, intraperitoneal, intravenous and subcutaneous), epidural, and mucosal (e.g., intranasal and oral routes). Administration may be by any suitable route, for example, by infusion or bolus injection, by absorption through epithelial or mucocutaneous linings (e.g., oral mucosa, rectal and intestinal mucosa, and the like), and may be by pulmonary administration (e.g., use of an inhaler or nebulizer, and formulation with an aerosolizing agent). An effective amount may be delivered by liposomes, microparticles and/or microcapsules in certain implementations.

An effective dosage of an active ingredient can be determined by assessing its in vitro activity in a cell or tissue system and/or in vivo activity in an animal system. For example, methods for extrapolating an effective dosage in mice and other animals to humans are known (see, for example, U.S. Pat. No. 4,938,949). Such systems can be used for determining the LD₅₀ (the dose lethal to 50% of the population) and the ED₅₀ (the dose therapeutically effective in 50% of the population) of an active ingredient. The dose ratio between a toxic and therapeutic effect is the therapeutic index and it can be expressed as the ratio ED₅₀/LD₅₀. A dosage of an active ingredient often lies within a range of circulating concentrations for which the ED₅₀ is associated with low toxicity or no toxicity. The dosage may vary within this range depending upon the dosage form employed and the route of administration utilized. A therapeutically effective dose of an active ingredient can be estimated initially from cell culture assays. A dose sometimes is formulated to achieve a circulating plasma concentration range covering the IC₅₀ (for example, the concentration of an active ingredient that achieves a half-maximal inhibition of a symptom) as determined in in vitro assays, as such information often is used to more accurately determine useful doses in humans. Levels in plasma may be measured, for example, by high performance liquid chromatography and/or a spectrometric process (for example, liquid chromatography and mass spectrometry (for example, LCMS)).

Another example of determining an effective dose for a subject is to directly assay levels of “free” and “bound” levels of an active ingredient in the serum of the test subject. Such assays may utilize antibody mimics and/or “biosensors” generated by molecular imprinting techniques. The active ingredient is used as a template, or “imprinting molecule”, to spatially organize polymerizable monomers prior to their polymerization with catalytic reagents. Subsequent removal of the imprinted molecule leaves a polymer matrix that contains a repeated “negative image” of the active ingredient and is able to selectively rebind the molecule under biological assay conditions (see, for example, Ansell, et al., Current Opinion in Biotechnology (1996) 7:89-94 and in Shea, Trends in Polymer Science (1994) 2:166-173).

Such “imprinted” affinity matrixes are amenable to ligand-binding assays, whereby the immobilized monoclonal antibody component is replaced by an appropriately imprinted matrix (see, for example, Vlatakis, et al., Nature (1993) 361:645-647). Through the use of isotope-labeling, “free” concentration of an active ingredient can be readily monitored and used in calculations of IC₅₀. Such “imprinted” affinity matrixes can also be designed to include fluorescent groups having photon-emitting properties that measurably change upon local and selective binding of an active ingredient. These changes can be readily assayed in real time using appropriate fiberoptic devices, in turn allowing the dose in a test subject to be quickly optimized based on its individual IC₅₀. An example of such a “biosensor” is addressed in Kriz, et al, Analytical Chemistry (1995) 67:2142-2144.

Non-limiting examples of doses include milligram or microgram amounts of an active ingredient per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (for example, a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific active ingredient employed, the age, body weight, general health, gender, and diet of the subject, the time of administration, the route of administration, the rate of excretion, any drug combination, and the degree of expression or activity to be modulated.

### EXAMPLES OF CERTAIN EMBODIMENTS

Listed hereafter are non-limiting embodiments.

A1. A compound of Formula A1 or Formula A2:

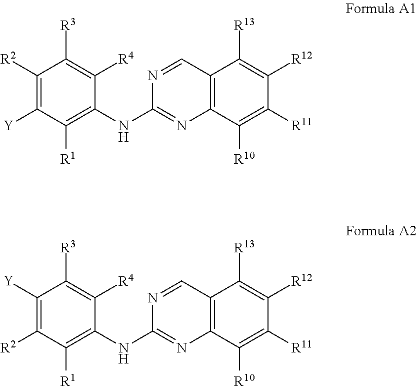

or a pharmaceutically acceptable salt, amide or ester thereof, where:
R¹, R², R³ and R⁴ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino or amido;
Y is —N(R^{b})C(O)—R^{Y}, —C(O)N(R^{b})—R^{YA}, —N(R^{b})—CH₂—R^{Y}; —CH₂—N(R^{b})R^{Y}, —N(R^{a})C(O)—R^{V}—N(R^{b})R^{Y}, —N(R^{b})C(O)—R^{V}—R^{Y}, —N(R^{a})R^{b} or of Formula F;
R^{a} is hydrogen, optionally substituted alkyl, optionally substituted alkynyl or of Formula F:

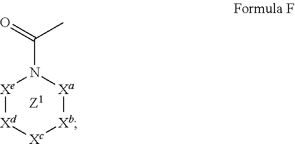

R^{b} is hydrogen or optionally substituted alkyl;
R^{V} is an optionally substituted alkylene;
R^{Y} is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted cycloalkylalkyl, optionally substituted heterocycloalkylalkyl, optionally substituted aryl, optionally substituted arylaminoalkyl, optionally substituted aryloxyalkyl, optionally substituted arylthioalkyl or optionally substituted arylalkyl, optionally substituted heteroaryl, optionally substituted heteroarylaminoalkyl, optionally substituted heteroaryloxyalkyl, optionally substituted heteroarylthioalkyl, optionally substituted heteroarylalkyl or is of Formula F;
R^{YA} is an optionally substituted aryl, optionally substituted arylaminoalkyl, optionally substituted aryloxyalkyl, optionally substituted arylthioalkyl or optionally substituted arylalkyl, optionally substituted heteroaryl, optionally substituted heteroarylaminoalkyl, optionally substituted heteroaryloxyalkyl, optionally substituted heteroarylthioalkyl or optionally substituted heteroarylalkyl;
Z¹ is an optionally substituted heterocycloalkyl, X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A}; and R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R¹⁰, R¹¹, R¹² and R¹³ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino, amido, R^{W} or —W—R^{W};
R^{W} is an optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl;
W is an optionally substituted alkylene, optionally substituted alkynyl, amino, amido, —O—, —S—, —S(O)— or —SO₂—;
R^{z} is hydrogen or R^{u}; and
R^{u} is an optionally substituted alkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl.

A2. The compound of embodiment A1, with the proviso that it is not of Formula X1, not of Formula X2, not of Formula X3 and not of Formula X4:

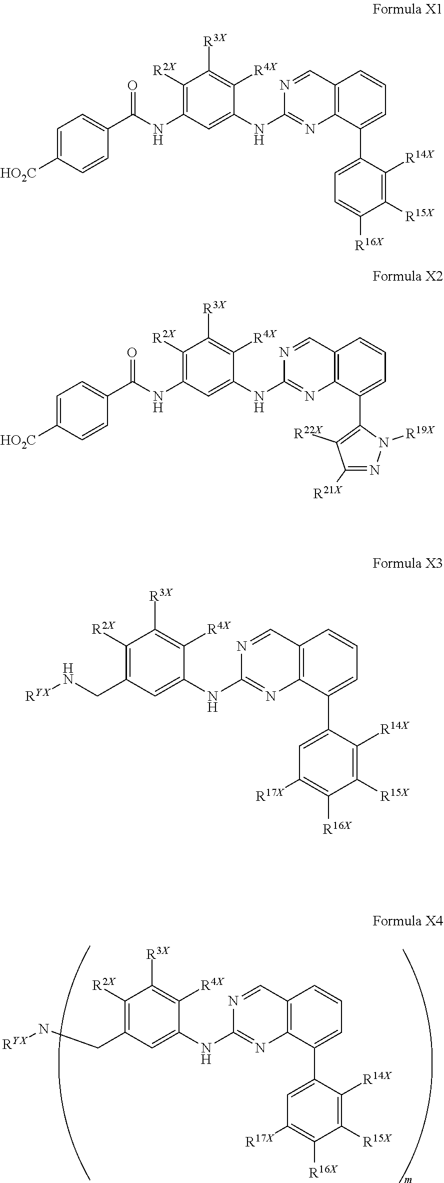

where: R^{2X}, R^{3X} and R^{4X} each independently is hydrogen, optionally substituted C1-C4 alkyl or optionally substituted C1-C4 alkoxy; R^{14X}, R^{15X}, R^{16X} and R^{17X} each independently is hydrogen, optionally substituted C1-C4 alkyl, optionally substituted C1-C4 alkoxy, or halo; R^{19X}, R^{21X} and R^{22X} each independently is hydrogen or optionally substituted C1-C6 alkyl; m is an integer of 1 or 2; R^{YX} is methyl or

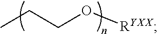

n is an integer of 1 to 10; and R^{YXX} is hydrogen, optionally substituted alkyl or optionally substituted amidoalkyl.

A3. The compound of embodiment A1 or A2, where Y is —N(R^{b})C(O)—R^{Y} or —C(O)N(R^{b})—R^{YA}, and R^{Y} and R^{YA} each is an optionally substituted phenyl.

A4. The compound of embodiment A3, which is of Formula A1-1, Formula A1-2 or Formula A2-1:

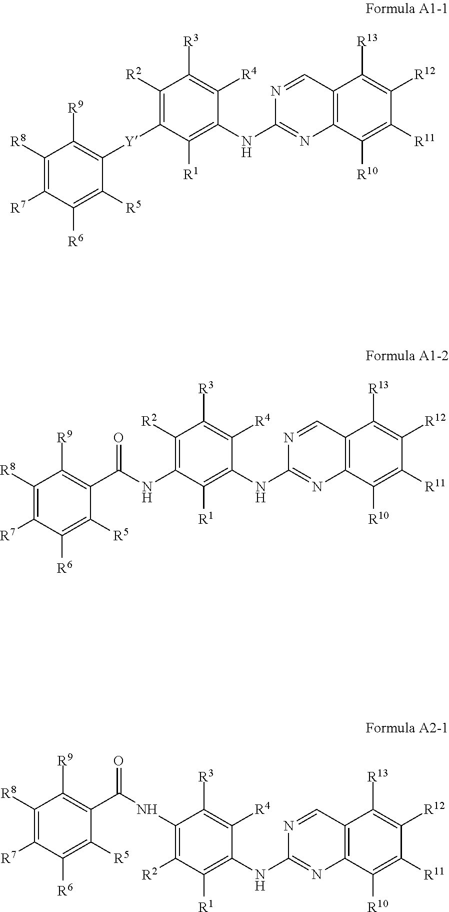

or a pharmaceutically acceptable salt, amide or ester thereof, where:
Y′ is —N(R^{b})C(O)— or —C(O)N(R^{b})—; and
R⁵, R⁶, R⁷, R⁸, and R⁹ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —B(OH)₂, —C(O)R^{z}, —C(O)OH, —C(O)OR^{u}, hydroxy, halo, cyano, nitro, amino, amido, optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl or a Formula F group.

A5. The compound of any one of embodiments A1-A4, where R¹⁰ is R^{W} and R^{W} is an optionally substituted phenyl or optionally substituted pyrazolyl.

A6. The compound of embodiment A5, which is of Formula A1-3, Formula A1-4, Formula A1-5, Formula A1-6, Formula A2-2 or Formula A2-3:

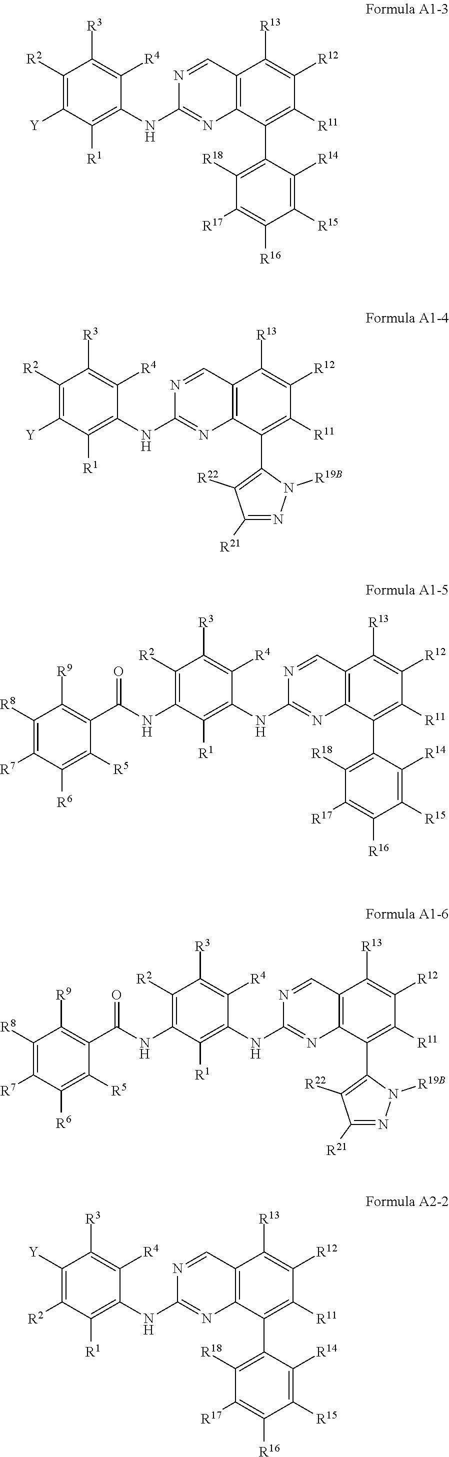

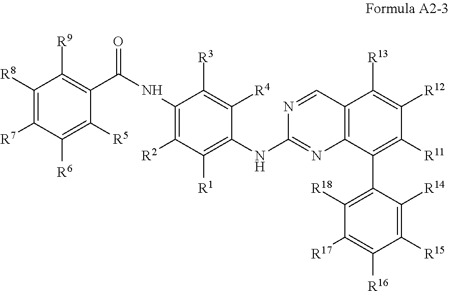

or a pharmaceutically acceptable salt, amide or ester thereof, where: R¹⁴, R¹⁵, R¹⁶, R¹⁷, R¹⁸, R^{19B}, R²¹ and R²² each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl.

A7. The compound of any one of embodiments A1-A4, where R¹⁰ is —W—R^{W}, W is —C≡C—, and R^{W} is an optionally substituted phenyl.

A8. The compound of embodiment A7, which is of Formula A1-7 or Formula A1-8:

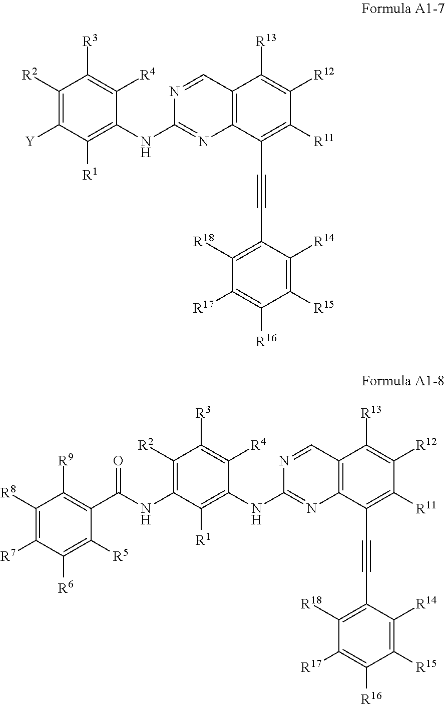

or a pharmaceutically acceptable salt, amide or ester thereof, where: R¹⁴, R¹⁵, R¹⁶, R¹⁷ and R¹⁸ each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl.

A9. The compound of any one of embodiments A1-A8, which is a pharmaceutically acceptable salt, or optionally a pharmaceutically acceptable hydrochloride salt.

A10. The compound of any one of embodiments A1-A9, where R^{Y} and R^{YA} each is an optionally substituted aryl, optionally substituted arylalkyl, optionally substituted heteroaryl or optionally substituted heteroarylalkyl.

A11. The compound of embodiment A10, where the optionally substituted aryl, optionally substituted heteroaryl, optionally substituted aryl of the optionally substituted arylalkyl, or optionally substituted heteroaryl or the optionally substituted heteroarylalkyl, is of Formula B1:

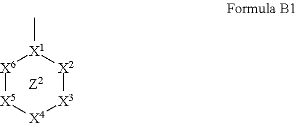

where: Z² is aryl or heteroaryl; X¹ independently is C or N; X² independently is C—R⁵, N—R^{5B} or N; X³ independently is C—R⁶, N—R^{6B} or N; X⁴ independently is C—R⁷, N—R^{7B} or N; X⁵ independently is C—R⁸, N—R^{8B} or N; and X⁶ independently is C—R⁹, N—R^{9B} or N; R⁶, R⁷, R⁸, R⁹, R^{6B}, R^{7B}, R^{8B} and R^{9B} are as defined herein; and optionally two adjacent R⁶, R⁷, R⁸, R⁹, R^{6B}, R^{7B}, R^{8B} and R^{9B} are linked in an optionally substituted aryl or optionally substituted heteroaryl.

A12. The compound of embodiment A11, where Z² is an optionally substituted pyridinyl, optionally substituted pyrimidinyl, optionally substituted pyrazinyl, optionally substituted pyridazinyl or optionally substituted triazinyl.

A13. The compound of embodiment A11 or A12, where:
X¹ is C, X² is C—R⁵, X³ is C—R⁶, X⁴ is C—R⁷, X⁵ is C—R⁸, and X⁶ is C—R⁹;
X¹ is C, X² is C—R⁵, X³ is N, X⁴ is C—R⁷, X⁵ is C—R⁸, and X⁶ is C—R⁹;
X¹ is C, X² is N, X³ is C—R⁶, X⁴ is N, X⁵ is C—R⁸, and X⁶ is C—R⁹;
X¹ is C, X² is C—R⁵, X³ is N, X⁴ is C—R⁷, X⁵ is N, and X⁶ is C—R⁹; or
X¹ is C, X² is C—R⁵, X³ is C—R⁶, X⁴ is N, X⁷ is C—R⁸ and X⁶ is C—R⁹.

A14. The compound of embodiment A10, where the optionally substituted aryl, optionally substituted heteroaryl, optionally substituted aryl of the optionally substituted arylalkyl, or optionally substituted heteroaryl or the optionally substituted heteroarylalkyl, is of Formula C1:

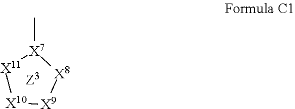

where: Z³ is aryl or heteroaryl; X⁷ independently is C or N; X⁸ independently is C—R⁵, N—R^{5B}, N, O, S, S(O) or SO₂; X⁹ independently is C—R⁶, N—R^{6B}, N, O, S, S(O) or SO₂; X¹⁰ independently is C—R⁷, N—R^{7B}, N, O, S, S(O) or SO₂; and X¹¹ independently is C—R⁸, N—R^{8B}, N, O, S, S(O) or SO₂; R⁵, R⁶, R⁷, R⁸, R^{5B}, R^{6B}, R^{7B} and R^{8B} are as defined herein; and optionally two adjacent R⁵, R⁶, R⁷, R⁸, R^{5B}, R^{6B}, R^{7B} and R^{8B} are linked in an optionally substituted aryl or optionally substituted heteroaryl.

A15. The compound of embodiment A14, where Z³ is an optionally substituted pyrrolyl, optionally substituted imidazolyl, optionally substituted pyrazolyl, optionally substituted puranyl, optionally substituted thiophenyl, optionally substituted oxazolyl, optionally substituted thiazolyl, optionally substituted isoxazolyl or optionally substituted isothiazolyl.

A16. The compound of embodiment A14 or A15, where:
X⁷ is C; X⁸ is N or N—R^{5B}; X⁹ is N or N—R^{6B}; X¹⁰ is C—R⁷; and X¹¹ is C—R⁸;
X⁷ is C; X⁸ is N or N—R^{5A}; X⁹ is C—R⁶; X¹⁰ is C—R⁷; and X¹¹ is C—R⁸;
X⁷ is C; X⁸ is N or N—R^{5B}; X⁹ is C—R⁶; X¹⁰ is C—R⁷; and X¹ is N or N—R^{8B};
X⁷ is C; X⁸ is N; X⁹ is C—R⁶; X¹⁰ is C—R⁷; and X¹¹ is S;
X⁷ is C; X⁸ is N; X⁹ is C—R⁶; X¹⁰ is C—R⁷; and X¹¹ is O; or
X⁷ is C; X⁸ is N; X⁹ is C—R⁶; X¹⁰ is O; and X¹¹ is C—R⁸.

A17. The compound of any one of embodiments A1-A9, where R^{Y} is an optionally substituted cycloalkyl, optionally substituted cycloalkylalkyl, optionally substituted heterocycloalkyl or optionally substituted heterocycloalkylalkyl.

A18. The compound of embodiment A17, where the optionally substituted heterocycloalkyl, or the optionally substituted heterocycloalkyl of the optionally substituted heterocycloalkylalkyl, contains 4, 5 or 6 ring atoms.

A19. The compound of embodiment A17 or A18, where the optionally substituted heterocycloalkyl, or the optionally substituted heterocycloalkyl of the optionally substituted heterocycloalkylalkyl, is an optionally substituted piperidinyl, optionally substituted piperazinyl, optionally substituted tetrahydropyran, or optionally substituted morpholinyl, optionally substituted pyrrolidinyl, optionally substituted imidazolidinyl, optionally substituted pyrazolidinyl or optionally substituted azetidinyl.

A20. The compound of any one of embodiments A17-A19, where R^{Y} comprises a substituted heterocycloalkyl, a C1-C6 alkyl substituted heterocycloalkyl, a methyl substituted heterocycloalkyl or a mono-methyl substituted heterocycloalkyl.

A21. The compound of any one of embodiments A17-A20, where the optionally substituted cycloalkyl or optionally substituted heterocycloalkyl is of Formula D1:

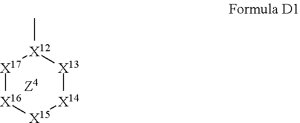

where: Z⁴ is cycloalkyl or heterocycloalkyl; X¹² is C—R^{aA}, C or N; X¹³ independently is C(R⁵)R^{5A}, C—R⁵, N—R^{5B}, N, O, S, S(O) or SO₂; X¹⁴ independently is C(R⁶)R^{6A}, C—R⁶, N—R^{6B}, N, O, S, S(O) or SO₂; X¹⁵ independently is C(R⁷)R^{7A}, C—R⁷, N—R^{7B}, N, O, S, S(O) or SO₂; X¹⁶ independently is C(R⁸)R^{8A}, C—R⁸, N—R^{8B}, N, O, S, S(O) or SO₂; and X¹⁷ independently is C(R⁹)R^{9A}, C—R⁹, N—R^{9B}, N, O, S, S(O) or SO₂; and optionally two adjacent R⁵, R^{5A}, R⁶, R^{6A}, R⁷, R^{7A}, R⁸, R^{8A}, R⁹ and R^{9A} are linked in an optionally substituted cycloalkyl or optionally substituted heterocycloalkyl.

A22. The compound of embodiment A21, where Z⁴ is an optionally substituted piperidinyl, optionally substituted piperazinyl, optionally substituted morpholinyl or optionally substituted tetrahydropyranyl.

A23. The compound of embodiment A21 or A22, where:
X¹² is C—R^{aA}; X¹³ is C—R⁵; X¹⁴ is C(R⁶)R^{6A}; X¹⁵ is N—R^{7B}; X¹⁶ is C(R⁸)R^{8A}; and X¹⁷ is C(R⁹) R^{9A};
X¹² is N; X¹³ is C(R⁵)R^{5A}; X¹⁴ is C(R⁶)R^{6A}; X¹⁵ is N—R^{7B}, X¹⁶ is C(R⁸)R^{8A}, and X¹⁷ is C(R⁹) R^{9A};
X¹² is N; X¹³ is C(R⁵)R^{5A}; X¹⁴ is C(R⁶)R^{6A}; X¹⁵ is C(R⁷)R^{7A}, X¹⁶ is C(R⁸)R^{8A}, and X¹⁷ is C(R⁹)R^{9A}.
X¹² is C—R^{a}; X¹³ is C(R⁵)R^{5A}; X¹⁴ is C(R⁶)R^{6A}; X¹⁵ is N—R^{7B}; X¹⁶ is C(R⁸)R^{8A}; and X¹⁷ is C(R⁹)R^{9A}; or
X¹² is C—R^{aA}; X¹³ is C(R⁵)R^{5A}; X¹⁴ is C(R⁶)R^{6A}; X¹⁵ is O; X¹⁶ is C(R^{s})R^{8A}; and X¹⁷ is C(R⁹)R^{9A}.

A24. The compound of any one of embodiments A17-A20, where the optionally substituted cycloalkyl or optionally substituted heterocycloalkyl is of Formula E1:

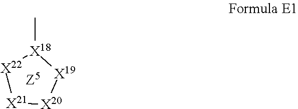

where: Z⁵ is cycloalkyl or heterocycloalkyl; X¹⁸ is C—R^{aA}, C or N; X¹⁹ independently is C(R⁵)R^{5A}, C—R⁵, N—R^{5B}, N, O, S, S(O) or SO₂; X²⁰ independently is C(R⁶)R^{6A}, C—R⁶, N—R^{6B}, N, O, S, S(O) or SO₂; X²¹ independently is C(R⁷)R^{7A}, C—R⁷, N—R^{7B}, N, O, S, S(O) or SO₂; and X²² independently is C(R⁸)R^{8A}, C—R⁸, N—R^{8B}, N, O, S, S(O) or SO₂; and optionally two adjacent R⁵, R^{5A}, R⁶, R^{6A}, R⁷, R^{7A}, R⁸, R^{8A}, R^{5B}, R^{6B}, R^{7B} and R^{8B} are linked in an optionally substituted cycloalkyl or optionally substituted hetero heterocycloalkyl.

A25. The compound of embodiment A24, where Z⁵ is an optionally substituted pyrrolidinyl, optionally substituted pyrazolidinyl or optionally substituted imidazolidinyl.

A26. The compound of embodiment A24 or A25, where X¹⁸ is C—R^{aA}, X¹⁹ is N—R^{5B}, X²⁰ is C(R⁶)R^{6A}, X²¹ is C(R⁷)R^{7A} and X²² is C(R⁸)RsA.

A27. The compound of any one of embodiments A4-A26, where:
R^{aA}, R⁵, R^{5A}, R⁶, R^{6A}, R⁷, R^{7A}, R⁸, R^{8A}, R⁹ and R^{9A} each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; and
R^{5B}, R^{6B}, R^{7B}, R^{8B}, and R^{9B} each independently is hydrogen, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl; and

A28. The compound of any one of embodiments A4-A27, where: R^{aA}, R⁵, R^{5A}, R⁶, R^{6A}, R⁷, R^{7A}, R⁸, R^{8A}, R⁹ and R^{9A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{c}C(O)N(R^{d})—, —C(O)N(R^{c}R^{d}), —NR^{e}R^{f}, —C(O)R^{z}, —C(O)OH, —C(O)OR^{u}, hydroxy, halo, cyano, nitro, optionally substituted C5-C6 cycloalkyl, optionally substituted heterocycloalkyl containing 5 or 6 ring member atoms; and R^{c}, R^{d}, R^{e} and R^{f} each independently is hydrogen or optionally substituted C1-C6 alkyl.

A29. The compound of embodiment A28, where:
the optionally substituted C1-C6 haloalkyl is an optionally substituted C1-C4 haloalkyl, or trifluoromethyl; and/or
R^{c}, R^{d}, R^{e} and R^{f} each independently is hydrogen or methyl.

A30. The compound of any one of embodiments A1-A29, where R^{u} is an optionally substituted C1-C4 alkyl, butyl, tert-butyl, iso-butyl, propyl, iso-propyl, ethyl or methyl.

A31. The compound of any one of embodiments A4-A30, where R^{aA}, R⁵, R^{5A}, R⁶, R^{6A}, R⁷, R^{7A}, R⁸, R^{8A}, R⁹ and R^{9A} each independently is hydrogen, unsubstituted C1-C4 alkyl, Cl, F, CF₃, CD₃, unsubstituted C1-C4 alkoxy, methoxy, isopropyloxy or dimethylamino.

A32. The compound of any one of embodiments A4-A31, where R^{5B}, R^{6B}, R^{7B}, R^{8B}, and R^{9B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u}.

A33. The compound of any one of embodiments A4-A32, where R^{5B}, R^{6B}, R^{7B}, R^{8B}, and R^{9B} each independently is hydrogen, optionally substituted C1-C4 alkyl, butyl, tert-butyl, iso-butyl, propyl, iso-propyl, ethyl or methyl.

A34. The compound of any one of embodiments A1-A33, where Y is —N(R^{b})C(O)—R^{Y} or —N(R^{b})C(O)—R^{V}—R^{Y}.

A35. The compound of any one of embodiments A1-A34, where Y is:

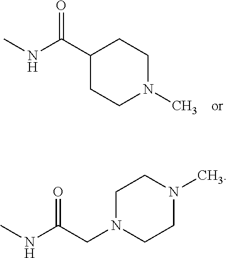

A36. The compound of any one of embodiments A4-A35, where one or more of R^{aA}, R⁵, R^{5A}, R⁶, R^{6A}, R⁷, R^{7A}, R⁸, R^{8A}, R⁹ and R^{9A} is —C(O)N(R^{c}R^{d}) and R^{c} and R^{d} each independently is hydrogen or optionally substituted C1-C4 alkyl.

A37. The compound of embodiment A36, where at least R⁷ is —C(O)N(R^{c}R^{d}).

A38. The compound of embodiment A36 or A37, where R^{c} and R^{d} each is unsubstituted C1-C4 alkyl.

A39. The compound of any one of embodiments A36-A38, where at least R⁷ is —C(O)N(CH₃)CH₃, —C(O)N(H)CH₃ or —C(O)NH₂.

A40. The compound of any one of embodiments A4-A39, where one or more of R^{aA}, R⁵, R^{5A}, R⁶, R^{6A}, R⁷, R^{7A}, R⁸, R^{8A}, R⁹ and R^{9A} is —C(O)OH or —C(O)OR^{u}.

A41. The compound of embodiments A40, where at least R⁷ is —C(O)OH or —C(O)OR^{u}.

A41.1. The compound of embodiments A40 or A41, where at least R⁷ is —C(O)OH.

A42. The compound of any one of embodiments A4-A41, where one or more of R^{aA}, R⁵, R^{5A}, R⁶, R^{6A}, R⁷, R^{7A}, R⁸, R^{8A}, R⁹ and R^{9A} is an optionally substituted C1-C4 haloalkyl, or a trifluoromethyl.

A43. The compound of embodiment A42, where one or two of R⁶, R⁷ and R⁸ each is, or R⁸ is, an optionally substituted C1-C4 haloalkyl, or a trifluoromethyl.

A44. The compound of any one of embodiments A4-A43, where at least one of R^{aA}, R⁵, R^{5A}, R⁶, R^{6A}, R⁷, R^{7A}, R⁸, R^{8A}, R⁹ and R^{9A} is an optionally substituted heterocycloalkyl or optionally substituted heterocycloalkylalkyl.

A45. The compound of embodiment A44, where one of R^{aA}, R^{s}, R^{5A}, R⁶, R^{6A}, R⁷, R^{7A}, R⁸, R^{8A}, R⁹ and R^{9A} is an optionally substituted heterocycloalkylalkyl containing 5 or 6 ring atoms.

A46. The compound of embodiment A45, where one of R^{aA}, R⁵, R^{5A}, R⁶, R^{6A}, R⁷, R^{7A}, R⁸, R^{8A}, R⁹ and R^{9A} is an optionally substituted heterocycloalkylalkyl containing 6 ring atoms and the alkyl attached to the heterocycloalkyl group is an unsubstituted C1-C4 alkylene, ethylene or methylene.

A47. The compound of embodiment A46, where one of R^{aA}, R^{s}, R^{5A}, R⁶, R^{6A}, R⁷, R^{7A}, R⁸, R^{8A}, R⁹ and R^{9A}, or at least R⁷, contains an optionally substituted piperazinyl or optionally substituted piperidinyl, or is:

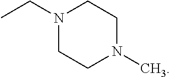

A48. The compound of any one of embodiments A4-A47, where one or more of R^{aA}, R⁵, R^{5A}, R⁶, R^{6A}, R⁷, R^{7A}, R⁸, R^{8A}, R⁹ and R^{9A} is an optionally substituted heteroaryl or optionally substituted heteroarylalkyl.

A49. The compound of embodiment A48, where one of R^{aA}, R⁵, R^{5A}, R⁶, R^{6A}, R⁷, R^{7A}, R⁸, R^{5A}, R⁹ and R^{9A} is an optionally substituted heteroaryl containing 5 or 6 ring atoms.

A50. The compound of embodiment A49, where one of R^{aA}, R⁵, R^{5A}, R⁶, R^{6A}, R⁷, R^{7A}, R⁸, R^{8A}, R⁹ and R^{9A}, or at least R⁶, contains an optionally substituted pyrrolyl, optionally substituted imidazolyl or optionally substituted pyrazolyl, or is:

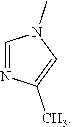

A51. The compound of any one of embodiments A4-A50, where:
one or more of R⁵, R^{5A}, R⁶, R^{6A}, R⁷, R^{7A}, R⁸, R^{8A}, R⁹ and R^{9A} is a Formula F group;
R⁷ is a Formula F group;
Y is a Formula F group;
Y is —N(R^{b})—CH₂—R^{Y} and R^{Y} is a Formula F group;
Y is —N(R^{a})R^{b}, R^{a} is a Formula F group and R^{b} is hydrogen, optionally substituted C1-C6 alkyl, optionally substituted C1-C4 alkyl, butyl, tert-butyl, iso-butyl, propyl, iso-propyl, ethyl or methyl; or
Y is —N(R^{b})C(O)—R^{V}—R^{Y} and R^{Y} is a Formula F group.

A52. The compound of any one of embodiments A1-A51, where R^{V} is an optionally substituted C1-C4 alkylene, —CH₂CH₂— or —CH₂—.

A53. The compound of any one of embodiments A1-A52, where R^{b} is hydrogen, optionally substituted C1-C4 alkyl, butyl, tert-butyl, iso-butyl, propyl, iso-propyl, ethyl or methyl.

A54. The compound of any one of embodiments A1-A53, which contains a Formula F group, where:
Z¹ is an optionally substituted piperidinyl, optionally substituted piperazinyl or optionally substituted morpholinyl;
Z¹ is an unsubstituted piperidinyl, unsubstituted piperazinyl or unsubstituted morpholinyl;
Z¹ is a substituted piperidinyl, substituted piperazinyl or substituted morpholinyl containing one or more C1-C6 alkyl substituents, or containing one or more C1-C4 alkyl substituents, or containing one or more ethyl or methyl substituents, or containing one methyl substituent;
X^{a} is C(R⁴³)R^{43A}; X^{b} is C(R⁴⁴)R^{44A}; X^{c} is N—R^{45B}; X^{d} is C(R⁴⁶)R^{46A}; and X^{e} is C(R⁴⁷)R^{47A};
X^{a} is C(R⁴³)R^{43A}; X^{b} is C(R⁴⁴)R^{44A}; X^{c} is O; X^{d} is C(R⁴⁶)R^{46A}; and X^{e} is C(R⁴⁷)R^{47A};
X^{a} is C(R⁴³)R^{43A}; X^{b} is C(R⁴⁴)R^{44A}; X^{c} is C(R⁴⁵)R^{45A}; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A} and R^{47A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{45B} is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl;
R⁴³, R⁴⁴, R⁴⁵, R⁴⁶, R⁴⁷, R^{43A}, R^{44A}, R^{45A}, R^{46A} and R^{47A} each independently is hydrogen, unsubstituted C1-C4 alkyl, Cl, F, CF₃, CD₃, unsubstituted C1-C4 alkoxy, methoxy, isopropyloxy or dimethylamino;
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C4 alkyl, ethyl or methyl;
R⁴³, R⁴⁴, R⁴⁵, R⁴⁶, R⁴⁷, R^{43A}, R^{44A}, R^{46A} and R^{47A} each is hydrogen and R^{45A} and R^{45B} each is hydrogen or optionally substituted C1-C6 alkyl, optionally substituted C1-C4 alkyl, ethyl or methyl;
R⁴³, R⁴⁴, R⁴⁵, R⁴⁶, R⁴⁷, R^{43A}, R^{44A}, R^{46A} and R^{47A} each is hydrogen and R^{45A} and R^{45B} is methyl; and/or
the Formula F group is:

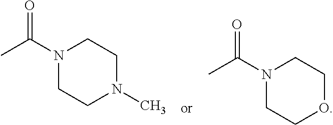

A55. The compound of any one of embodiments A1-A9, where Y is —N(R^{b})C(O)—R^{Y} or —N(R^{b})C(O)—R^{V}—R^{Y}; and R^{Y} is hydrogen, optionally substituted alkyl, optionally substituted alkynyl, optionally substituted alkoxy, optionally substituted heteroalkyl, optionally substituted haloalkyl, optionally substituted deuteroalkyl, optionally substituted hydroxyalkyl, optionally substituted mercaptoalkyl or optionally substituted aminoalkyl;

A56. The compound of embodiment A55, where R^{Y} is an optionally substituted C1-C6 alkyl, optionally substituted C1-C4 alkyl, butyl, tert-butyl, iso-butyl, propyl, iso-propyl, ethyl or methyl.

A56.1. The compound of embodiment A55, where R^{Y} is an optionally substituted C1-C6 alkynyl, optionally substituted C1-C4 alkynyl or —C≡CH.

A57. The compound of any one of embodiments A1-A9, where Y is —N(R^{b})C(O)—R^{Y} or —N(R^{b})C(O)—R^{V}—R^{Y}, R^{Y} is an optionally substituted alkenyl.

A58. The compound of embodiment A57, where R^{Y} is —CH═CH₂.

A59. The compound of any one of embodiments A1-A9, where Y is —N(R^{a})C(O)—R^{V}—N(R^{b})R^{Y}; and R^{Y} is hydrogen or optionally substituted alkyl.

A60. The compound of embodiment A59, where R^{Y} is hydrogen, optionally substituted C1-C4 alkyl, butyl, tert-butyl, iso-butyl, propyl, iso-propyl, ethyl or methyl.

A61. The compound of any one of embodiments A55-A60, where R^{V} is an optionally substituted C1-C4 alkylene, —CH₂CH₂— or —CH₂—.

A62. The compound of any one of embodiments A55-A61, where R^{b} is hydrogen or optionally substituted C1-C4 alkyl, butyl, tert-butyl, iso-butyl, propyl, iso-propyl, ethyl or methyl.

A63. The compound of any one of embodiments A1-A9, where Y is —N(R^{a})R^{b} and R^{a} and R^{b} each is hydrogen.

A64. The compound of any one of embodiments A1-A63, where:
at least one of, or one of, R¹⁰, R¹¹, R¹² and R¹³ is R^{W} or —W—R^{W};
R¹⁰ is R^{W} or —W—R^{W} and R¹¹, R¹² and R¹³ each is not R^{W} or —W—R^{W};
R¹¹ is R^{W} or —W—R^{W} and R¹⁰, R¹² and R¹³ each is not R^{W} or —W—R^{W};
R¹² is R^{W} or —W—R^{W} and R¹⁰, R¹¹ and R¹³ each is not R^{W} or —W—R^{W};
R¹³ is R^{W} or —W—R^{W} and R¹⁰, R¹¹ and R¹² each is not R^{W} or —W—R^{W}; or
R¹⁰, R¹¹, R¹² and R¹³ each is not R^{W} or —W—R^{W}.

A64.1. The compound of any one of embodiments A1-A63, where:
R¹⁰ is R^{W} or —W—R^{W} and R¹¹, R¹² and R¹³ each is not R^{W} or —W—R^{W}; or
R¹¹ is R^{W} or —W—R^{W} and R¹⁰, R¹² and R¹³ each is not R^{W} or —W—R^{W}.

A65. The compound of embodiment A64 or A64.1, where the R¹⁰, R¹¹, R¹² and R¹³ that is not R^{W} or —W—R^{W} each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino or amido.

A66. The compound of any one of embodiments A64, A64.1 or A65, where the R¹⁰, R¹¹, R¹² and R¹³ that is not R^{W} or —W—R^{W} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, hydroxy, halo, cyano or nitro; and R^{g}, R^{h}, R^{j} and R^{k} each independently is hydrogen or optionally substituted C1-C6 alkyl.

A67. The compound of embodiment A66, where R^{g}, R^{h}, R^{j} and R^{k} each independently is hydrogen, optionally substituted C1-C4 alkyl, butyl, iso-butyl, tert-butyl, propyl, isopropyl, ethyl or methyl.

A68. The compound of embodiment A66 or A67, where R^{u} is an optionally substituted C1-C4 alkyl, butyl, iso-butyl, tert-butyl, propyl, isopropyl, ethyl or methyl.

A69. The compound of any one of embodiments A64-A68, where the R¹⁰, R¹¹, R¹² and R¹³ that is not R^{W} or —W—R^{W} each independently is hydrogen, optionally substituted C1-C4 alkyl, optionally substituted C1-C4 alkoxy, ethyl, ethoxy, methyl, methoxy, chloro, fluoro, bromo, iodo, CF₃ or CDs.

A70. The compound of any one of embodiments A64-A69, where one of R¹⁰, R¹¹, R¹² and R¹³ is R^{W} or —W—R^{W}, and one, two or three of R¹⁰, R¹¹, R¹² and R¹³ that is not R^{W} or —W—R^{W} each is hydrogen.

A71. The compound of any one of embodiments A64-A70, where R¹⁰ is R^{W} or —W—R^{W} and R¹¹, R¹² and R¹³ each is not R^{W} or —W—R^{W}.

A71.1. The compound of any one of embodiments A64-A70, where R¹¹ is R^{W} or —W—R^{W} and R¹⁰, R¹² and R¹³ each is not R^{W} or —W—R^{W}.

A72. The compound of any one of embodiments A64-A71.1, where at least one of, or one of, R¹⁰, R¹¹, R¹² and R¹³ is —W—R^{W}.

A73. The compound of any one of embodiments A64-A72, where R¹⁰ is —W—R^{W} and R¹¹, R¹² and R¹³ each is not R^{W} or —W—R^{W}.

A74. The compound of any one of embodiments A1-A73, where W is —CH₂—, —C≡C—, —NH(R^{t})—, —O—, —S—, —S(O)— or —SO₂—, and R^{t} is hydrogen or optionally substituted C1-C6 alkyl, optionally substituted C1-C4 alkyl, ethyl or methyl.

A75. The compound of any one of embodiments A1-A74, where at least one of, or one of, R¹⁰, R¹¹, R¹² and R¹³ is R^{W} or —W—R^{W}, and R^{W} is an optionally substituted aryl, optionally substituted arylalkyl, optionally substituted heteroaryl or optionally substituted heteroarylalkyl.

A76. The compound of embodiment A75, where R^{W} comprises an optionally substituted aryl or optionally substituted heteroaryl of Formula B2:

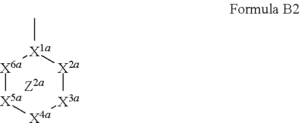

where: Z^{2a} is aryl or heteroaryl; X^{1a} independently is C or N; X^{2a} independently is C—R¹⁴, N—R^{14B} or N; X^{3a} independently is C—R¹⁵, N—R^{15B} or N; X^{4a} independently is C—R¹⁶, N—R^{16B} or N; X^{5a} independently is C—R¹⁷, N—R^{17B} or N; and X^{6a} independently is C—R¹⁸, N—R^{18B} or N; and optionally two adjacent R¹⁴, R¹⁵, R¹⁶, R¹⁷, R¹⁸, R^{14B}, R^{15B}, R^{16B}, R^{17B} and R^{18B} are linked in an optionally substituted aryl or optionally substituted heteroaryl.

A77. The compound of embodiment A76, where Z^{2a} is an optionally substituted pyridinyl, optionally substituted pyrimidinyl, optionally substituted pyrazinyl, optionally substituted pyridazinyl or optionally substituted triazinyl, and optionally, R^{W} is an optionally substituted indolyl or optionally substituted benzooxazolyl.

A78. The compound of embodiment A76 or A77, where:
X^{1a} is C, X^{2a} is C—R¹⁴, X^{3a} is C—R¹⁵, X^{4a} is C—R¹⁶, X^{5a} is C—R¹⁷ and X^{6a} is C—R¹⁸;
X^{1a} is C, X^{2a} is C—R¹⁴, X^{3a} is N, X^{4a} is C—R¹⁶, X^{5a} is C—R¹⁷, and X^{6a} is C—R¹⁸;
X^{1a} is C, X^{2a} is N, X^{3a} is C—R¹⁵, X^{4a} is N, X^{5a} is C—R¹⁷, and X^{6a} is C—R¹⁸;
X^{1a} is C, X^{2a} is C—R¹⁴, X^{3a} is N, X^{4a} is C—R¹⁶, X^{5a} is N, and X^{6a} is C—R¹⁸; or
X^{1a} is C, X^{2a} is C—R⁴, X^{3a} is C—R¹⁵, X^{4a} is N, X^{5a} is C—R¹⁷ and X^{6a} is C—R¹⁸

A78.1. The compound of any one of embodiments A76-A78, where:
X^{1a} is C, X^{2a} is C—R¹⁴, X^{3a} is C—R¹⁵, X^{4a} is C—R¹⁶, X^{5a} is C—R¹⁷ and X^{6a} is C—R¹⁸, and R¹⁶ and R¹⁷ together are joined as a fused optionally substituted aryl, optionally substituted heteroaryl, optionally substituted cycloalkyl or optionally substituted heterocycloalkyl containing five ring member atoms;
the fused ring is an optionally substituted heteroaryl containing five ring atoms; a
the fused ring is an optionally substituted pyrrolyl or oxazolyl;
R^{W} is an optionally substituted benzo-oxazolyl group or an optionally substituted benzo[d]oxazol-5-yl group; and/or
R^{W} is an optionally substituted indolyl group or optionally substituted 1H-indol-5-yl group.

A78.2. The compound of any one of embodiments A76-A78, where:
X^{1a} is C, X^{2a} is C—R¹⁴, X^{3a} is N, X^{4a} is C—R¹⁶, X^{5a} is C—R¹⁷, and X^{6a} is C—R¹⁸, where the X^{3a} nitrogen and R¹⁶ together are joined as a fused optionally substituted aryl or optionally substituted heteroaryl containing five ring member atoms; and/or
R^{W} is an optionally substituted imidazo-pyridinyl group or an optionally substituted imidazo[1,2-a]pyridin-6-yl group.

A79. The compound of embodiment A75, where R^{W} comprises an optionally substituted aryl or optionally substituted heteroaryl according of Formula C2:

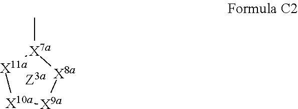

where: Z^{3a} is aryl or heteroaryl; X^{7a} independently is C or N; X^{8a} independently is C—R¹⁹, N—R^{19B}, N, O, S, S(O) or SO₂; X^{9a} independently is C—R²⁰, N—R^{20B}, N, O, S, S(O) or SO₂; X^{10a} independently is C—R²¹, N—R^{21B}, N, O, S, S(O) or SO₂; and X^{11a} independently is C—R²², N—R^{22B}, N, O, S, S(O) or SO₂; and optionally two adjacent R¹⁹, R²⁰, R²¹, R²², R^{19B}, R^{20B}, R^{21B} and R^{22B} are linked in an optionally substituted aryl or optionally substituted heteroaryl.

A80. The compound of embodiment A79, where Z^{3a} is an optionally substituted pyrrolyl, optionally substituted imidazolyl, optionally substituted pyrazolyl, optionally substituted puranyl, optionally substituted thiophenyl, optionally substituted oxazolyl, optionally substituted thiazolyl, optionally substituted isoxazolyl or optionally substituted isothiazolyl, or optionally R^{W} is an optionally substituted indolyl.

A81. The compound of embodiment A79 or A80, where:
X^{7a} is C; X^{8a} is N or N—R^{19B}; X^{9a} is N or N—R^{20B}; X^{10a} is C—R²¹; and X^{11a} is C—R²²;
X^{7a} is C; X^{8a} is N or N—R^{19B}; X^{9a} is C—R²⁰; X^{10a} is C—R²¹; and X^{11a} is C—R²²
X^{7a} is C; X^{8a} is N or N—R^{19B}; X^{9a} is C—R²⁰; X^{10a} is C—R²¹; and X^{11a} is N or N—R^{22B};
X^{7a} is C; X^{8a} is N; X^{9a} is C—R²⁰; X^{10a} is C—R²¹; and X^{11a} is S; or
X^{7a} is C; X^{8a} is N; X^{9a} is C—R²⁰; X^{10a} is O; and X^{11a} is C—R²².

A81.1. The compound of any one of embodiments A79-A81, where:
X^{7a} is C; X^{8a} is N or N—R^{19B}; X^{9a} is C—R²⁰; X^{10a} is C—R²¹; and X^{11a} is C—R²² and R²⁰ and R²¹ together are joined as a fused optionally substituted aryl, optionally substituted heteroaryl, optionally substituted cycloalkyl or optionally substituted heterocycloalkyl containing six ring member atoms; and/or
R^{W} is an optionally substituted indolyl group or an optionally substituted 1H-indol-2-yl group.

A82. The compound of any one of embodiments A1-A81.1, where at least one of, or one of, R¹⁰, R¹¹, R¹² and R¹³ is R^{W} or —W—R^{W}, and R^{W} is an optionally substituted cycloalkyl, optionally substituted cycloalkylalkyl, optionally substituted heterocycloalkyl or an optionally substituted heterocycloalkylalkyl.

A83. The compound of embodiment A82, where the optionally substituted heterocycloalkyl, or the optionally substituted heterocycloalkyl of the optionally substituted heterocycloalkylalkyl, contains 4, 5 or 6 ring atoms.

A84. The compound of embodiment A82 or A83, where R^{W} comprises an optionally substituted piperidinyl, optionally substituted piperazinyl, optionally substituted tetrahydropyran, or optionally substituted morpholinyl, optionally substituted pyrrolidinyl, optionally substituted imidazolidinyl, optionally substituted pyrazolidinyl or optionally substituted azetidinyl.

A85. The compound of any one of embodiments A82-A84, where R^{W} comprises a substituted heterocycloalkyl, a C1-C6 alkyl substituted heterocycloalkyl, a methyl substituted heterocycloalkyl or a mono-methyl substituted heterocycloalkyl.

A86. The compound of any one of embodiments A82-A85, where R^{W} comprises an optionally substituted cycloalkyl or optionally substituted heterocycloalkyl of Formula D2:

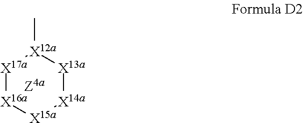

where: Z^{4a} is cycloalkyl or heterocycloalkyl; X^{12a} is C—R^{aB}, C or N; X^{13a} independently is C(R¹⁴)R^{14A}, C—R¹⁴, N—R^{14B}, N, O, S, S(O) or SO₂; X^{14a} independently is C(R¹⁵)R^{15A}, C—R¹⁵, N—R^{15B}, N, O, S, S(O) or SO₂; X^{15a} independently is C(R¹⁶)R^{16A}, C—R¹⁶, N—R^{16B}, N, O, S, S(O) or SO₂; X^{16a} independently is C(R¹⁷)R^{17A}, C—R¹⁷, N—R^{17B}, N, O, S, S(O) or SO₂; and X^{17a} independently is C(R¹⁸)R^{18A}, C—R¹⁸, N—R^{18B}, N, O, S, S(O) or SO₂; and optionally two adjacent R¹⁴, R¹⁵, R¹⁶, R¹⁷, R¹⁸, R^{14A}, R^{15A}, R^{16A}, R^{17A}, R^{18A}, R^{14B}, R^{15B}, R^{16B}, R^{17B} and R^{18B} are linked and joined in an optionally substituted cycloalkyl or optionally substituted heterocycloalkyl.

A87. The compound of embodiment A86, where, Z^{4a} is an optionally substituted piperidinyl, optionally substituted piperazinyl, optionally substituted morpholinyl or optionally substituted tetrahydropyridinyl.

A88. The compound of embodiment A86 or A87, where:
X^{12a} is C; X^{13a} is C—R¹⁴; X^{14a} is C(R¹⁵)R^{15A}; X^{15a} is N—R^{16B}; X^{16a} is C(R¹⁷)R^{17A}; and X^{17a} is C(R¹⁸)R^{18A};
X^{12a} is N; X^{13a} is C(R¹⁴)R^{14A}; X^{14a} is C(R¹⁵)R^{15A}; X^{15a} is N—R^{16B}; X^{16a} is C(R¹⁷)R^{17A}; and X^{17a} is C(R¹⁸)R^{18A};
X^{12a} is N; X^{13a} is C(R¹⁴)R^{14A}; X^{14a} is C(R¹⁵)R^{15A}; X^{15a} is C(R¹⁶)R^{16A}; X^{16a} is C(R¹⁷)R^{17A}; and X^{17a} is C(R¹⁸)R^{18A};
X^{12a} is C—R^{aB}; X^{13a} is C(R¹⁴)R^{14A}; X^{14a} is C(R¹⁵)R^{15A}; X^{15a} is N—R^{16B}; X^{16a} is C(R¹⁷)R^{17A} and X^{17a} is C(R¹⁸)R^{18A}; or
X^{12a} is C—R^{aB}; X^{13a} is C(R¹⁴)R^{14A}; X^{14a} is C(R¹⁵) R^{15A}; X^{15a} is O; X^{16a} is C(R¹⁷)R^{17A}; and X^{17a} is C(R¹⁸)R^{18A}.

A89. The compound of any one of embodiments A82-A85, where R^{W} comprises an optionally substituted cycloalkyl or optionally substituted heterocycloalkyl of Formula E2:

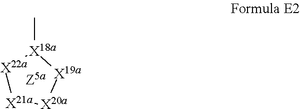

where: Z^{5a} is cycloalkyl or heterocycloalkyl; X^{18a} is C—R^{aB}, C or N; X^{19a} independently is C(R¹⁹)R^{19A}, C—R¹⁹, N—R^{19B}, N, O, S, S(O) or SO₂; X^{20a} independently is C(R²⁰)R^{20A}, C—R²⁰, N—R^{20B}, N, O, S, S(O) or SO₂; X^{21a} independently is C(R²¹)R^{21A}, C—R²¹, N—R^{21B}, N, O, S, S(O) or SO₂; and X^{22a} independently is C(R²²)R^{22A}, C—R²², N—R^{22B}, N, O, S, S(O) or SO₂; R^{aB}, R¹⁹, R^{19A}, R²⁰, R^{20A}, R²¹, R^{21A}, R²², R^{22A}, R^{19B}, R^{20B}, R^{21B} and R^{22B} are as defined herein; and optionally two adjacent R^{aB}, R¹⁹, R^{19A}, R²⁰, R²⁰, R²¹, R^{21A}, R²², R^{22A}, R^{19B}, R^{20B}, R^{21B} and R^{22B} are linked in an optionally substituted cycloalkyl or optionally substituted heterocycloalkyl.

A90. The compound of embodiment A89, where Z^{5a} is an optionally substituted pyrazolidinyl, optionally substituted imidazolidinyl or optionally substituted pyrazolidinyl.

A91. The compound of embodiment A89 or A91, where X^{18a} is C—R^{aB}; X^{19a} is N—R^{19B}; X^{20a} is C(R²⁰)R^{20A}; X^{21a} is C(R²¹)R^{21A}; and X^{22a} is C(R²²)R^{22A}.

A92. The compound of any one of embodiments A76-A91, where R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{20A}, R²¹, R^{21A}, R²² and R^{22A} each independently is hydrogen optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, hydroxy, halo, cyano, nitro, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; and
R^{14B}, R^{15B}, R^{16B}, R^{17B}, R^{18B}, R^{19B}, R^{20B}, R^{21B} and R^{22B} each independently is hydrogen, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl, or —C(O)OR^{u}, where R^{u} is defined for Formula A1.

A93. The compound of any one of embodiments A76-A92, where R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{20A}, R²¹, R^{21A}, R²² and R^{22A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, hydroxy, halo, cyano, nitro, optionally substituted C5-C6 cycloalkyl, optionally substituted heterocycloalkyl containing 5 or 6 ring member atoms; and R^{g}, R^{h}, R^{j} and R^{k} each independently is hydrogen or optionally substituted C1-C6 alkyl.

A94. The compound of embodiment A93, where R^{g}, R^{h}, R^{j}, and R^{k} each independently is hydrogen, optionally substituted C1-C4 alkyl, butyl, iso-butyl, tert-butyl, propyl, isopropyl, ethyl or methyl; and/or R^{u} is an optionally substituted C1-C4 alkyl, butyl, iso-butyl, tert-butyl, propyl, isopropyl, ethyl or methyl.

A95. The compound of any one of embodiments A76-A94, where R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{20A}, R²¹, R^{21A}, R²² and R^{22A} each independently is hydrogen, optionally substituted C1-C4 alkylamine, optionally substituted C1-C4 hydroxyalkylamine, HO—CH₂CH₂—N(H)—, halo, fluoro, chloro, optionally substituted C1-C4 alkyl, butyl, tert-butyl, iso-butyl, propyl, iso-propyl, ethyl, methyl, optionally substituted C1-C4 alkoxy, substituted C1-C4 alkoxy, unsubstituted benzyloxy, isopropyloxy, ethoxy, methoxy, nitro, —C(O)R^{z}, —C(O)OR^{u} or —C(O)OH.

A96. The compound of any one of embodiments A76-A95, where:
R¹⁶ is an optionally substituted C1-C4 alkylamine, optionally substituted C1-C4 hydroxyalkylamine, or HO—CH₂CH₂—N(H)—; R¹⁵ or R¹⁶ is halo, fluoro or chloro;
one, two or three of R¹⁴, R¹⁶ and R¹⁸ is an optionally substituted C1-C4 alkyl, ethyl, methyl, optionally substituted C1-C4 alkoxy, isopropyloxy, ethoxy or methoxy;
R¹⁶ is hydroxy;
R¹⁴ is —C(O)R^{z};
R¹⁴ is —C(O)H;
R¹⁴ or R¹⁸ is unsubstituted pyrrolidinyl;
R¹⁶ is an optionally substituted C1-C4 alkoxy, substituted C1-C4 alkoxy, or unsubstituted benzyloxy;
R¹⁵ is nitro; and/or
R¹⁷ is —C(O)OH.

A97. The compound of any one of embodiments A76-A96, where R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R^{20A}, R²¹, R^{21A}, R²² and R^{22A} each independently is hydrogen, unsubstituted C1-C4 alkyl, Cl, F, CF₃, CD₃, unsubstituted C1-C4 alkoxy, methoxy, isopropyloxy or dimethylamino.

A98. The compound of any one of embodiments A76-A97, where R^{14B}, R^{15B}, R^{16B}, R^{17B}, R^{18B}, R^{19B}, R^{20B}, R^{21B} and R^{22B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u}, where R^{u} is an optionally substituted C1-C6 alkyl, optionally substituted C1-C4 alkyl, butyl, tert-butyl, iso-butyl, propyl, iso-propyl, ethyl or methyl.

A99. The compound of any one of embodiments A76-A98, where R^{14B}, R^{15B}, R^{16B}, R^{17B}, R^{18B}, R^{19B}, R^{20B}, R^{21B} and R^{22B} each independently is hydrogen, optionally substituted C1-C6 alkyl, optionally substituted C1-C4 alkyl, butyl, tert-butyl, iso-butyl, propyl, iso-propyl, ethyl or methyl.

A100. The compound of any one of embodiments A1-A99, where R¹, R², R³ and R⁴ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, hydroxy, halo, cyano or nitro; and R^{p}, R^{q}, R^{r} and R^{s} each independently is hydrogen or optionally substituted C1-C6 alkyl.

A101. The compound of embodiment A100, where R^{p}, R^{q}, R^{r} and R^{s} each independently is hydrogen, optionally substituted C1-C4 alkyl, butyl, iso-butyl, tert-butyl, propyl, isopropyl, ethyl or methyl.

A102. The compound of embodiment A100 or A101, where R^{u} is an optionally substituted C1-C4 alkyl, butyl, iso-butyl, tert-butyl, propyl, isopropyl, ethyl or methyl.

A103. The compound of any one of embodiments A1-A102, where R¹, R², R³ and R⁴ each independently is hydrogen, optionally substituted C1-C4 alkyl, optionally substituted C1-C4 alkoxy, ethyl, ethoxy, methyl, methoxy, Cl, F, CF₃ or CD₃.

A104. The compound of any one of embodiments A1-A103, where R¹ is hydrogen.

A105. The compound of any one of embodiments A1-A104, where one of R², R³ and R⁴ is an optionally substituted C1-C4 alkyl, optionally substituted C1-C4 alkoxy, ethyl, ethoxy, methyl, methoxy, Cl, F, CF₃ or CD₃, and the other two of R², R³ and R⁴ each is hydrogen.

A106. The compound of any one of embodiments A1-A104, where:
two, three or four of R¹, R², R³ and R⁴ each is hydrogen;
R² is methyl, ethyl, methoxy or ethoxy, and R¹, R³ and R⁴ each is hydrogen;
R³ is methyl, ethyl, methoxy or ethoxy, and R¹, R² and R⁴ each is hydrogen; or
R⁴ is methyl, ethyl, methoxy or ethoxy, and R¹, R² and R³ each is hydrogen.

A107. The compound of any one of embodiments A1-A104, where R² is hydrogen, optionally substituted C1-C4 alkyl, optionally substituted C1-C4 deuteroalkyl, optionally substituted C1-C4 haloalkyl, optionally substituted C1-C4 alkylamino, halo or unsubstituted C1-C4 alkoxy; or R² is hydrogen, unsubstituted C1-C4 alkyl, unsubstituted C1-C4 deuteroalkyl, unsubstituted C1-C4 haloalkyl, unsubstituted C1-C4 alkylamino, or halo.

A108. The compound of any one of embodiments A1-A104, where R² is hydrogen, unsubstituted C1-C4 alkyl, Cl, F, CF₃, CD₃, unsubstituted C1-C4 alkoxy, methoxy, isopropyloxy or dimethylamino; or R² is unsubstituted C1-C4 alkyl, ethyl or methyl.

A109. The compound of any one of embodiments A1-A108, where:
one, two, three or four of R¹⁰, R¹¹, R¹² and R¹³ each is hydrogen;
one, two or three of R³, R⁴ and R⁵ each is hydrogen;
one, two or three of R¹⁵, R¹⁷ and R¹⁸ each is hydrogen;
one or two of R¹⁴ and R¹⁶ each is hydrogen;
one or both of R²¹ and R²² each is hydrogen; and/or one, two, three or four of R⁵, R⁶, R⁸ and R⁹ each is hydrogen.

B1. A compound of Formula A1-3:

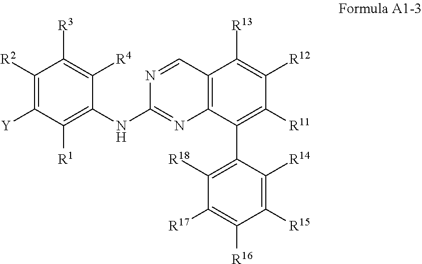

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano; Y is —N(R^{b})C(O)—R^{Y} or —N(R^{b})C(O)—R^{V}—R^{Y};
R^{Y} is an optionally substituted heterocycloalkyl containing six ring atoms;
R^{V} is a substituted C1-C4 alkylene or unsubstituted C1-C4 alkylene;
R^{b}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

B2. The compound of embodiment B1, where R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

B3. The compound of embodiment B1 or B2, where R¹, R², R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

B4. The compound of any one of embodiments B1-B3, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

B5. The compound of any one of embodiments B1-B4, where:
one, two, three or four of R¹, R¹¹, R¹² and R¹³ each is hydrogen;
one, two or three of R¹⁵, R¹⁷ and R¹⁸ each is hydrogen;
one or two of R³ and R⁴ each is hydrogen; and/or
R^{b} is hydrogen.

B6. The compound of any one of embodiments B1-B5, where R² is methyl, ethyl, methoxy or ethoxy, and R¹, R³ and R⁴ each is hydrogen.

B7. The compound of any one of embodiments B1-B6, where R^{V} is unsubstituted ethylene or unsubstituted methylene.

B8. The compound of any one of embodiments B1-B7, where (i) R^{p}, R^{q}, R^{r} and R^{s} each independently is hydrogen or methyl; and/or (ii) R^{u} is ethyl or methyl.

B9. The compound of any one of embodiments B1-B8, where R^{Y} is an optionally substituted piperidinyl, optionally substituted piperazinyl or optionally substituted morpholinyl.

B10. The compound of any one of embodiments B1-B9, where R^{Y} is a substituted piperidinyl or substituted piperazinyl, which optionally is substituted by an optionally substituted C1-C6 alkyl, ethyl or methyl at one, two or three ring atoms.

B11. The compound of any one of embodiments B1-B9, where R^{Y} is an unsubstituted morpholinyl.

B12. The compound of any one of embodiments B1-B9, where R^{Y} is of Formula D1:

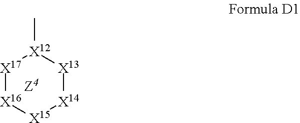

where:
X¹² is N; X¹³ is C(R⁵)R^{5A}; X¹⁴ is C(R⁶)R^{6A}; X¹⁵ is N—R^{7B}, X¹⁶ is C(R⁸)R^{8A}, and X¹⁷ is C(R⁹)R^{9A}; or
X¹² is C—R^{aA}; X¹³ is C(R⁵)R^{5A}; X¹⁴ is C(R⁶)R^{6A}; X¹⁵ is N—R^{7B}; X¹⁶ is C(R⁸)R^{8A}; and X¹⁷ is C(R⁹)R^{9A};
R^{aA}, R⁵, R^{5A}, R⁶, R^{6A}, R⁸, R^{8A}, R⁹ and R^{9A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{k}R^{j}, —C(O)R^{z}, —C(O)OH, —C(O)OR^{u}, —B(OH)₂, hydroxy, halo, nitro or cyano; R^{7B} is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u};
R^{g}, R^{h}, R^{k} and R^{j} each independently is hydrogen or optionally substituted C1-C6 alkyl; and R^{u} is an optionally substituted C1-C6 alkyl.

B12.1. The compound of embodiment B12, where Y is —N(R^{b})C(O)—R¹; X¹² is C—R^{a}; X¹³ is C(R⁵)R^{5A}; X¹⁴ is C(R⁶)R^{6A}; X¹⁵ is N—R^{7B}; X¹⁶ is C(R^{s})R^{8A}; and X¹⁷ is C(R⁹)R^{9A}.

B12.2. The compound of embodiment B12, where Y is —N(R^{b})C(O)—R^{V}—R^{Y}; X¹² is N; X¹³ is C(R⁵)R^{5A}; X¹⁴ is C(R⁶)R^{6A}; X¹⁵ is N—R^{7B}, X¹⁶ is C(R^{s})RsA, and X¹⁷ is C(R⁹)R^{9A}.

B12.3. The compound of any one of embodiments B12-B12.2, where (i) R^{aA}, R⁵, R^{5A}, R⁶, R^{6A}, R^{7B}, R⁸, R^{8A}, R⁹ and R^{9A} each independently is hydrogen or optionally substituted C1-C4 alkyl; (ii) R^{u} is an optionally substituted C1-C4 alkyl; (iii) R^{g}, R^{h}, R^{k} and R^{j} each independently is hydrogen or optionally substituted C1-C4 alkyl; (iv) the optionally substituted C1-C4 alkyl of (i), (ii) or (iii) is an unsubstituted C1-C4 alkyl; and/or (v) the unsubstituted C1-C4 alkyl of (iv) is butyl, tert-butyl, iso-butyl, propyl, isopropyl, ethyl or methyl.

B13. The compound of any one of embodiments B12-B12.3, where R^{aA}, R⁵, R^{5A}, R⁶, R^{6A}, R^{7B}, R⁸, R^{8A}, R⁹ and R^{9A} each independently is hydrogen or unsubstituted C1-C4 alkyl.

B14. The compound of any one of embodiments B12 to B13, where R^{aA}, R⁵, R^{5A}, R⁶, R^{6A}, R⁸, R^{8A}, R⁹ and R^{9A} each is hydrogen and R^{7B} is methyl.

B15. The compound of any one of embodiments B1-B10 and B12-B14, where Y is:

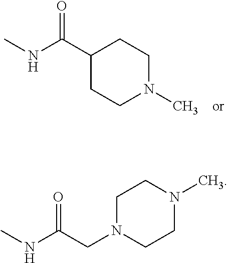

B15.1. A compound of formula:
N-(5-((8-(4-fluoro-2-isopropoxyphenyl)quinazolin-2-yl)amino)-2-methylphenyl)-1-methylpiperidine-4-carboxamide; or
(S)—N-(5-((8-(4-fluoro-2-isopropoxyphenyl)quinazolin-2-yl)amino)-2-methylphenyl)-1-methylpyrrolidine-2-carboxamide; or
N-(5-((8-(4-fluoro-2-isopropoxyphenyl)quinazolin-2-yl)amino)-2-methylphenyl)-1-methylazetidine-3-carboxamide; or
N-(5-((8-(4-fluoro-2-isopropoxyphenyl)quinazolin-2-yl)amino)-2-methylphenyl)tetrahydro-2H-pyran-4-carboxamide; or
(R)—N-(5-((8-(4-fluoro-2-isopropoxyphenyl)quinazolin-2-yl)amino)-2-methylphenyl)-1-methylpyrrolidine-2-carboxamide; or
N-(5-((8-(4-fluoro-2-isopropoxyphenyl)quinazolin-2-yl)amino)-2-methylphenyl)-2-(4-methylpiperazin-1-yl)acetamide; or
N-(5-((8-(4-fluoro-2-isopropoxyphenyl)quinazolin-2-yl)amino)-2-methylphenyl)-4-methylpiperazine-1-carboxamide; or
   or a pharmaceutically acceptable salt thereof.

B16. A compound of formula:
N-(5-((8-(4-fluoro-2-isopropoxyphenyl)quinazolin-2-yl)amino)-2-methylphenyl)-1-methylpiperidine-4-carboxamide; or
N-(5-((8-(4-fluoro-2-isopropoxyphenyl)quinazolin-2-yl)amino)-2-methylphenyl)-2-(4-methylpiperazin-1-yl)acetamide; or
   or a pharmaceutically acceptable salt thereof.

C1. A compound of Formula A1-3:

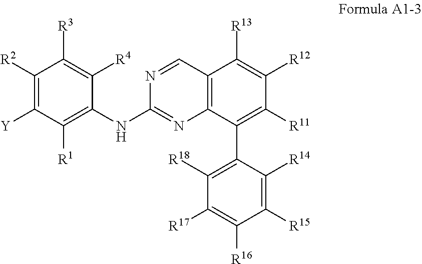

or a pharmaceutically acceptable salt, amide or ester thereof, where:
R¹, R², R³, R⁴, R¹¹, R¹², R¹³, R¹⁴, R¹⁵, R¹⁶, R¹¹ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{a})R^{b};
R^{a}, R^{b}, R^{p}, R^{q}, R^{r} and R^{s} each independently is hydrogen or optionally substituted C1-C6 alkyl;
R^{z} is hydrogen or R^{u}; and
R^{u} is or optionally substituted C1-C6 alkyl.

C2. The compound of embodiment C1, where R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy.

C3. The compound of embodiment C1 or C2, where R¹, R², R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

C4. The compound of embodiment C1 or C2, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

C5. The compound of any one of embodiments C1-C4, where:
one, two three or four of R¹, R¹¹, R¹² and R¹³ each is hydrogen;
one, two or three of R¹⁵, R¹⁷ and R¹⁸ each is hydrogen;
one or two of R³ and R⁴ each is hydrogen; and/or
R² is hydrogen.

C6. The compound of any one of embodiments C1-C5, where R² is methyl, ethyl, methoxy or ethoxy, and R¹, R³ and R⁴ each is hydrogen.

C7. The compound of any one of embodiments C1-C6, where (i) R^{p}, R^{q}, R^{r} and R^{s} each independently is hydrogen or methyl; and/or (ii) R^{u} is ethyl or methyl.

C8. The compound of any one of embodiments C1-C7, where R^{a} and R^{b} each independently is hydrogen or unsubstituted C1-C4 alkyl, ethyl or methyl.

C9. The compound of any one of embodiments C1-C8, where R^{a} and R^{b} each is hydrogen.

C10. The compound of any one of embodiments C1-C9, with the proviso that R^{a} and R^{b} are not joined and do not form an unsubstituted heterocycloalkyl or substituted heterocycloalkyl.

C11. A compound of formula:

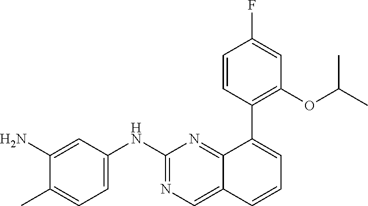 [SMILES: Cc1ccc(Nc2ncc3cccc(-c4ccc(F)cc4OC(C)C)c3n2)cc1N]

or a pharmaceutically acceptable salt thereof.

D1. A compound of Formula A1-3:

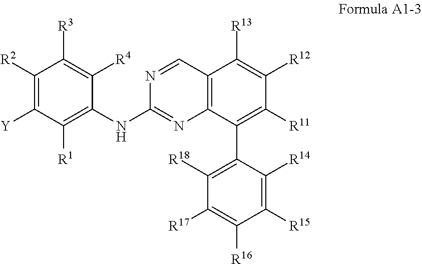

or a pharmaceutically acceptable salt, amide or ester thereof, where:
R¹, R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
Y is —N(R^{b})C(O)—R^{Y};
R^{Y} is an optionally substituted alkenyl;
R² is hydrogen, optionally substituted C1-C6 alkyl, unsubstituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —N(H)R^{r}, —NH₂, —C(O)R^{z}, —C(O)OH, —C(O)OR^{u}, —B(OH)₂, hydroxy, halo, nitro or cyano;
R^{b}, R^{g}, R^{h}, R^{j}, R^{k}, R^{p} and R^{q} each independently is hydrogen or optionally substituted C1-C6 alkyl;
R^{r} and R^{s} each independently is hydrogen or unsubstituted C1-C6 alkyl;
R^{z} is hydrogen or R^{u}; and
R^{u} is an optionally substituted C1-C6 alkyl.

D2. The compound of embodiment D1, where R^{Y} is an optionally substituted C₂-C₄ alkenyl.

D3. The compound of embodiment D1 or D2, where R^{Y} is —CH═CH₂.

D4. The compound of any one of embodiments D1-D3, where R¹, R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy.

D5. The compound of any one of embodiments D1-D4, where R¹, R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

D6. The compound of any one of embodiments D1-D4, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

D7. The compound of any one of embodiments D1-D6, where: R² is hydrogen, optionally substituted C1-C4 alkyl, optionally substituted C1-C4 deuteroalkyl, optionally substituted C1-C4 haloalkyl, optionally substituted C1-C4 alkylamino or halo;
R² is hydrogen, unsubstituted C1-C4 alkyl, unsubstituted C1-C4 deuteroalkyl, unsubstituted C1-C4 haloalkyl, unsubstituted C1-C4 alkylamino, or halo;
R² is hydrogen, unsubstituted C1-C4 alkyl, Cl, F, CF₃, CD₃ or dimethylamino;
R² is unsubstituted C1-C4 alkoxy, ethoxy or methoxy; and/or
R² is unsubstituted C1-C4 alkyl, ethyl or methyl.

D8. The compound of any one of embodiments D1-D7, where:
one, two, three or four of R¹, R¹¹, R¹² and R¹³ each is hydrogen;
one, two or three of R¹⁵, R¹⁷ and R¹⁸ each is hydrogen;
one or two of R³ and R⁴ each is hydrogen;
R² is hydrogen;
R^{b} is hydrogen; and/or
R^{b}, R^{k}, R^{m}, R^{p} and R^{q} each independently is hydrogen or methyl.

D9. The compound of any one of embodiments D1-D8, where R² is methyl, ethyl, methoxy or ethoxy, and R¹, R³ and R⁴ each is hydrogen.

D10. The compound of any one of embodiments D1-D9, where (i) R^{p}, R^{q}, R^{r} and R^{s} each independently is hydrogen or methyl; and/or (ii) R^{u} is ethyl or methyl.

D11. The compound of any one of embodiments D1-D12, with the proviso that R^{b} and R^{Y} are not joined and do not form an unsubstituted heterocycloalkyl or substituted heterocycloalkyl.

D12. A compound of formula:

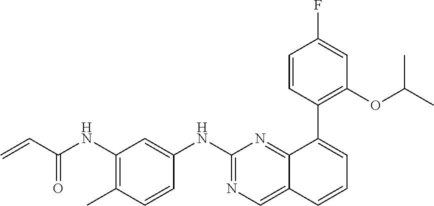 [SMILES: C=CC(=O)Nc1cc(Nc2ncc3cccc(-c4ccc(F)cc4OC(C)C)c3n2)ccc1C]

or a pharmaceutically acceptable salt thereof.

E1. A compound of Formula A1-8:

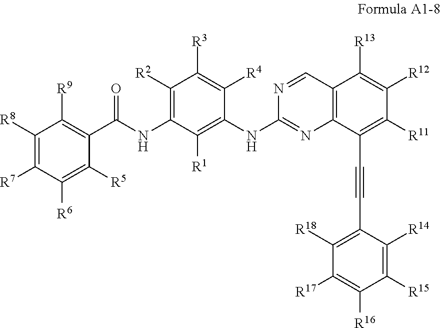

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
R⁵, R⁶, R⁷, R⁸ and R⁹ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{g}, R^{h}, R^{j}, R^{k}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

E2. The compound of embodiment E1, where R¹, R², R³, R⁴, R⁵, R⁶, R⁷, R⁸, R⁹, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

E3. The compound of embodiment E1 or E2, where R¹, R², R³, R⁴, R⁵, R⁶, R⁷, R⁸, R⁹, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

E4. The compound of any one of embodiments E1-E3, where:
one, two, three or four of R¹, R¹¹, R¹² and R¹³ each is hydrogen;
one, two of three of R¹⁵, R¹⁷ and R¹⁸ each is hydrogen;
one or two of R³ and R⁴ each is hydrogen;
one, two, three or four of R⁵, R⁶, R⁸ and R⁹ each is hydrogen; and/or R² is hydrogen.

E5. The compound of any one of embodiments E1-E4, where R² is methyl, ethyl, methoxy or ethoxy, and R¹, R³ and R⁴ each is hydrogen.

E6. The compound of any one of embodiments E1-E5, where R⁷ is —C(O)OH or —C(O)OR^{u}.

E6.1. The compound of any one of embodiments E1-E5, where R⁷ is —C(O)OH.

E7. The compound of any one of embodiments E1-E6, where R^{u} is an optionally substituted C1-C4 alkyl, ethyl or methyl.

E8. The compound of any one of embodiments E1-E7, where R⁷ is R^{p}C(O)N(R^{p})— or —C(O)N(R^{p}R^{q}) and R^{p} and R^{q} each independently is hydrogen, optionally substituted C1-C4 alkyl, ethyl or methyl.

E9. The compound of any one of embodiments E1-E8, where R^{g}, R^{h}, R^{j}, R¹, R^{p}, R^{q}, R^{r} and R^{s} each independently is hydrogen or methyl.

E10. The compound of any one of embodiments E1-E9, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is hydrogen, chloro or fluoro; and/or (v) R¹⁶ is hydrogen.

E11. A compound of formula:

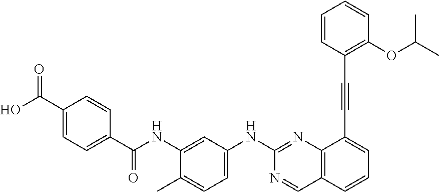 [SMILES: Cc1ccc(Nc2ncc3cccc(C#Cc4ccccc4OC(C)C)c3n2)cc1NC(=O)c1ccc(C(=O)O)cc1]

or a pharmaceutically acceptable amide, ester or salt thereof.

F1. A compound of Formula A1-5:

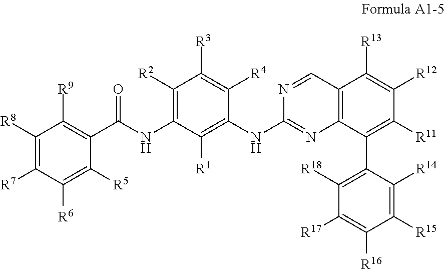

or a pharmaceutically acceptable salt, amide or ester thereof, where:
R⁷ is —C(O)N(R^{c}R^{d}) or is of Formula F:

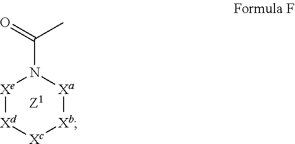

R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Z¹ is an optionally substituted heterocycloalkyl; X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{c}, R^{d}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

F2. The compound of embodiment F1, where R¹, R², R³, R⁴, R⁵, R⁶, R⁸, R⁹, R¹¹, R¹², R¹³, R¹⁴, R¹⁵, R¹⁶, R¹¹ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

F3. The compound of embodiment F1 or F2, where R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino;

F4. The compound of any one of embodiments F1-F3, where:
one, two, three or four of R¹, R¹¹, R¹² and R¹³ each is hydrogen;
one, two or three of R¹⁵, R¹⁷ and R¹⁸ each is hydrogen;
one or two of R³ and R⁴ each is hydrogen;
R² is hydrogen; and/or
one, two, three or four of R⁵, R⁶, R⁸ and R⁹ each is hydrogen.

F5. The compound of any one of embodiments F1-F4, where R² is methyl, ethyl, methoxy or ethoxy, and R¹, R³ and R⁴ each is hydrogen.

F6. The compound of any one of embodiments F1-F5, where R⁷ is —C(O)N(R^{c}R^{d}) and R^{c} and R^{d} each independently is hydrogen or optionally substituted C1-C4 alkyl.

F7. The compound of embodiments F6, where R^{c} and R^{d} each independently is hydrogen or unsubstituted C1-C4 alkyl; or R^{c} and R^{d} each independently is hydrogen or methyl; or R^{c} and R^{d} each is unsubstituted C1-C4 alkyl; or R^{c} and R^{d} each is methyl.

F8. The compound of embodiment F7, where R⁷ is —C(O)N(CH₃)CH₃, —C(O)N(H)CH₃ or —C(O)NH₂.

F9. The compound of any one of embodiments F1-F5, where R⁷ is a Formula F group.

F10. The compound of embodiment F9, where:
X^{a} is C(R⁴³)R^{43A}; X^{b} is C(R⁴⁴)R^{44A}; X^{c} is N—R^{45B}; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, ethyl or methyl;
R⁴³, R⁴⁴, R⁴⁵, R⁴⁶, R⁴⁷, R^{43A}, R^{44A}, R^{46A} and R^{47A} each is hydrogen and R^{45A} and R^{45B} each is hydrogen, optionally substituted C1-C6 alkyl, ethyl or methyl; and/or
R⁴³, R⁴⁴, R⁴⁵, R⁴⁶, R⁴⁷, R^{43A}, R^{44A}, R^{46A} and R^{47A} each is hydrogen and R^{45A} and R^{45B} is methyl.

F11. The compound of any one of embodiments F1-F10, where R⁷ is:

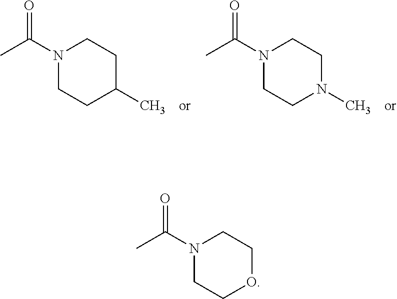

F12. The compound of any one of embodiments F1-F11, where R^{u} is an optionally substituted C1-C4 alkyl, butyl, tert-butyl, iso-butyl, propyl, isopropyl, ethyl or methyl.

F13. The compound of any one of embodiments F1-F12, where R^{p}, R^{q}, R^{r} and R^{s} each independently is hydrogen, ethyl or methyl.

F14. The compound of any one of embodiments F1-F13, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

F14.1. A compound of formula:
N-(5-((8-(4-fluoro-2-isopropoxyphenyl)quinazolin-2-yl)amino)-2-methylphenyl)terephthalamide; or
N1-(5-((8-(4-fluoro-2-isopropoxyphenyl)quinazolin-2-yl)amino)-2-methylphenyl)-N4-methylterephthalamide; or
N1-(5-((8-(4-fluoro-2-isopropoxyphenyl)quinazolin-2-yl)amino)-2-methylphenyl)-N4,N4-dimethylterephthalamide; or
N-(5-((8-(4-fluoro-2-isopropoxyphenyl)quinazolin-2-yl)amino)-2-methylphenyl)-4-(4-methylpiperazine-1-carbonyl)benzamide; or
N-(5-((8-(4-fluoro-2-isopropoxyphenyl)quinazolin-2-yl)amino)-2-methylphenyl)-4-(morpholine-4-carbonyl)benzamide; or
   or a pharmaceutically acceptable salt thereof.

F15. A compound of formula:
N1-(5-((8-(4-fluoro-2-isopropoxyphenyl)quinazolin-2-yl)amino)-2-methylphenyl)-N4,N4-dimethylterephthalamide; or
N-(5-((8-(4-fluoro-2-isopropoxyphenyl)quinazolin-2-yl)amino)-2-methylphenyl)-4-(4-methylpiperazine-1-carbonyl)benzamide; or
   or a pharmaceutically acceptable salt thereof.

G1. A compound of Formula A1-3:

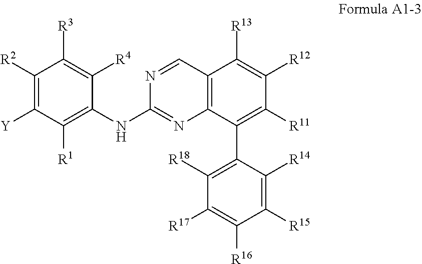

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{b})C(O)—R^{Y};
R^{Y} is an unsubstituted C1-C6 alkyl or unsubstituted C1-C6 deuteroalkyl;
R^{b}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

G2. The compound of embodiment G1, where R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

G3. The compound of embodiment G1 or G2, where R¹, R², R³, R⁴, R⁵, R⁶, R⁸, R⁹, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

G4. The compound of any one of embodiments G1-G3, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

G5. The compound of any one of embodiments G1-G4, where:
one, two, three or four of R¹, R¹¹, R¹² and R¹³ each is hydrogen;
one, two or three of R¹⁵, R¹⁷ and R¹⁸ each is hydrogen;
one or two of R³ and R⁴ each is hydrogen; and/or R^{b} is hydrogen.

G6. The compound of any one of embodiments G1-G5, where R² is methyl, ethyl, methoxy or ethoxy, and R¹, R³ and R⁴ each is hydrogen.

G7. The compound of any one of embodiments G1-G6, where (i) R^{V} is unsubstituted ethylene or unsubstituted methylene; (ii) R^{p}, R^{q}, R^{r} and R^{s} each independently is hydrogen or methyl; and/or
(iii) R^{u} is ethyl or methyl.

G8. The compound of any one of embodiments G1-G7, where R¹ is methyl or ethyl.

G9. The compound of any one of embodiments G1-G8, where R^{Y} is methyl.

G10. A compound of formula:

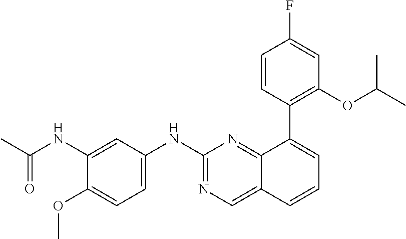 [SMILES: COc1ccc(Nc2ncc3cccc(-c4ccc(F)cc4OC(C)C)c3n2)cc1NC(C)=O]

or a pharmaceutically acceptable salt thereof.

G100. A compound of Formula A1-6:

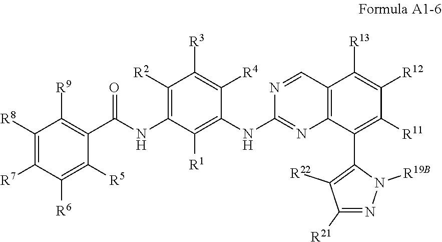

or a pharmaceutically acceptable salt, amide or ester thereof, where:
R¹, R², R³, R⁴, R¹¹, R¹², R¹³, R²¹ and R²² each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
R⁵, R⁶, R⁸ and R⁹ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R⁷ is R^{c}C(O)N(R^{d})— or —C(O)N(R^{c}R^{d});
each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{19B} is hydrogen, optionally substituted C1-C6 alkyl, optionally substituted C1-C4 alkyl, butyl, tert-butyl, iso-butyl, propyl, iso-propyl, ethyl or methyl;
R^{c}, R^{d}, R^{g}, R^{h}, R^{j}, R^{k}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

G101. The compound of embodiment G100, where R¹, R², R³, R⁴, R⁵, R⁶, R⁸, R⁹, R¹¹, R¹², R¹³, R²¹ and R²² each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

G102. The compound of embodiment G100 or G101, where R¹, R², R³, R⁴, R⁵, R⁶, R⁸, R⁹, R¹¹, R¹², R¹³, R²¹ and R²² each independently is hydrogen, unsubstituted C1-C4 alkyl, Cl, F, CF₃, CD₃, unsubstituted C1-C4 alkoxy, methoxy, isopropyloxy or dimethylamino.

G103. The compound of any one of embodiments G101-G103, where:
one, two, three or four of R¹, R¹¹, R¹² and R¹³ each is hydrogen;
one, two of three of R^{19B}, R²¹ and R²² each is hydrogen;
one or two of R³ and R⁴ each is hydrogen;
one, two, three or four of R⁵, R⁶, R⁸ and R⁹ each is hydrogen; and/or
R² is hydrogen.

G104. The compound of any one of embodiments G100-G103, where R² is methyl, ethyl, methoxy or ethoxy, and R¹, R³ and R⁴ each is hydrogen.

G105. The compound of any one of embodiments G100-G104, where R^{c} and R^{d} each independently is hydrogen or unsubstituted C1-C4 alkyl; or R^{c} and R^{d} each independently is hydrogen or methyl; or R^{c} is hydrogen and R^{d} is unsubstituted C1-C4 alkyl; or R^{c} is hydrogen and R^{d} is methyl; or R^{c} and R^{d} each is unsubstituted C1-C4 alkyl; or R^{c} and R^{d} each is methyl.

G106. The compound of embodiment G105, where R⁷ is —C(O)N(CH₃)CH₃, —C(O)N(H)CH₃ or —C(O)NH₂.

G107. The compound of embodiment G106, where R⁷ is —C(O)N(H)CH₃.

G108. The compound of any one of embodiments G100-G107, where R^{g}, R^{h}, R^{j}, R^{k}, R^{r} and R^{s} each independently is hydrogen or methyl.

G109. The compound of any one of embodiments G100-G108, where (i) R²¹ and R²² each independently is hydrogen, unsubstituted C1-C4 alkyl, Cl, F, CF₃, CD₃, unsubstituted C1-C4 alkoxy, methoxy, isopropyloxy or dimethylamino; (ii) one or two of R²¹ and R²² each is hydrogen; (iii) R^{19B} is an optionally substituted C1-C6 alkyl; (iv) R^{19B} is unsubstituted C1-C6 alkyl; and/or (v) R^{19B} is butyl, iso-butyl, tert-butyl, propyl, iso-propyl, ethyl or methyl.

G110. A compound of formula:

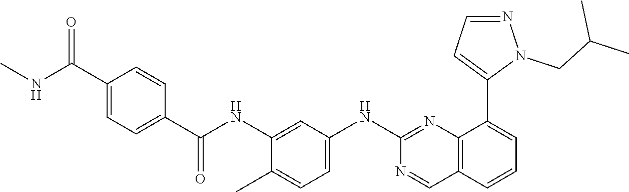 [SMILES: CNC(=O)c1ccc(C(=O)Nc2cc(Nc3ncc4cccc(-c5ccnn5CC(C)C)c4n3)ccc2C)cc1]

or a pharmaceutically acceptable amide, ester or salt thereof.

G200. A compound of Formula A1-3:

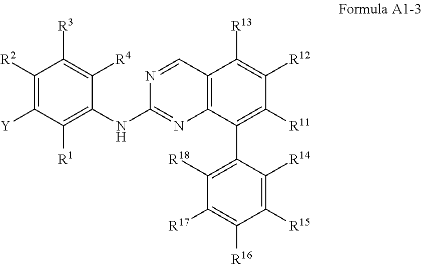

or a pharmaceutically acceptable salt, amide or ester thereof, where:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is of Formula F:

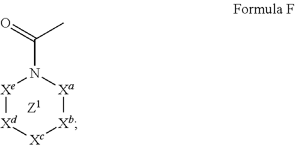

Z¹ is an optionally substituted heterocycloalkyl; X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{p}, R^{q}, R^{r} and R^{s} each independently is hydrogen or optionally substituted C1-C6 alkyl;
R^{z} is hydrogen or R^{u}; and
R^{u} is or optionally substituted C1-C6 alkyl.

G201. The compound of embodiment G200, where R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl, optionally substituted C1-C4 alkylamino, halo or optionally substituted C1-C4 alkoxy.

C202. The compound of embodiment G200 or G201, where R¹, R², R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

G203. The compound of embodiment G200 or G201, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

G204. The compound of any one of embodiments G200-G203, where R² is an optionally substituted C1-C4 alkoxy, optionally substituted C1-C4 alkyl or methyl; or R² is methyl, ethyl, methoxy or ethoxy, and R¹, R³ and R⁴ each is hydrogen.

G205. The compound of any one of embodiments G200-G204, where:
one, two three or four of R¹, R¹¹, R¹² and R¹³ each is hydrogen;
one, two or three of R¹⁵, R¹⁷ and R¹⁸ each is hydrogen;
one or two of R³ and R⁴ each is hydrogen; and/or
R² is hydrogen.

G206. The compound of any one of embodiments G200-G205, where R^{p}, R^{q}, R^{r} and R^{s} each independently is hydrogen, ethyl or methyl.

G207. The compound of any one of embodiments G200-G206, where:
X^{a} is C(R⁴³)R^{43A}; X^{b} is C(R⁴⁴)R^{44A}; X^{c} is N—R^{45B}; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, ethyl or methyl;
R⁴³, R⁴⁴, R⁴⁵, R⁴⁶, R⁴⁷, R^{43A}, R^{44A}, R^{46A} and R^{47A} each is hydrogen and R^{45A} and R^{45B} each is hydrogen, optionally substituted C1-C6 alkyl, ethyl or methyl; and/or
R⁴³, R⁴⁴, R⁴⁵, R⁴⁶, R⁴⁷, R^{43A}, R^{44A}, R^{46A} and R^{47A} each is hydrogen and R^{45A} and R^{45B} is methyl.

G208. The compound of embodiment G207, where Y is:

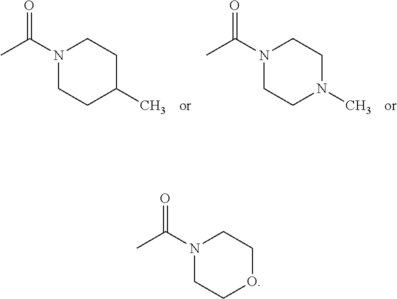

G209. A compound of formula:

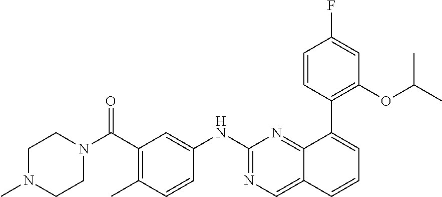 [SMILES: Cc1ccc(Nc2ncc3cccc(-c4ccc(F)cc4OC(C)C)c3n2)cc1C(=O)N1CCN(C)CC1]

or a pharmaceutically acceptable salt thereof.

G300. A compound of Formula A1-2:

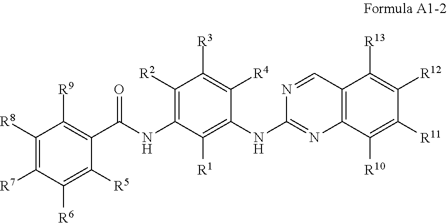

or a pharmaceutically acceptable salt, amide or ester thereof, where:
R¹⁰ is of Formula B2:

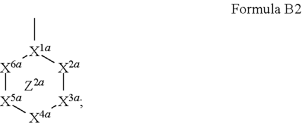

Z^{2a} is heteroaryl; and (i) X^{1a} is C, X^{2a} is C—R¹⁴, X^{3a} is N, X^{4a} is C—R¹⁶, X^{5a} is N, and X^{6a} is C—R¹⁸; or (ii) X^{1a} is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused optionally substituted heteroaryl containing five ring member atoms;
R¹, R², R³, R⁴, R⁵, R⁶, R⁷, R⁸, R⁹, R¹¹, R¹², R¹³, R¹⁴, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{p}, R^{q}, R^{r} and R^{s} each independently is hydrogen or optionally substituted C1-C6 alkyl;
R^{z} is hydrogen or R^{u}; and R^{u} is an optionally substituted C1-C6 alkyl.

G301. The compound of embodiment G300, where R¹, R², R³, R⁴, R⁵, R⁶, R⁸, R⁹, R¹¹, R¹², R¹³, R¹⁴, R¹⁶, R¹¹ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

G302. The compound of embodiment G300 or G301, where R¹, R², R³, R⁴, R⁵, R⁶, R⁸, R⁹, R¹¹, R¹², R¹³, R¹⁴, R¹⁶, R¹¹ and R¹⁸ each independently is hydrogen, unsubstituted C1-C4 alkyl, Cl, F, CF₃, CD₃, unsubstituted C1-C4 alkoxy, methoxy, isopropyloxy or dimethylamino;

G303. The compound of any one of embodiments G300-G302, where:
one, two, three or four of R¹, R¹¹, R¹² and R¹³ each is hydrogen;
one or two of R¹⁷ and R¹⁸ each is hydrogen;
one or two of R³ and R⁴ each is hydrogen;
R² is hydrogen; and/or
one, two, three or four of R⁵, R⁶, R⁸ and R⁹ each is hydrogen.

G304. The compound of any one of embodiments G300-G303, where R² is methyl, ethyl, methoxy or ethoxy, and R¹, R³ and R⁴ each is hydrogen.

G305. The compound of any one of embodiments G300-G304, where R⁷ is —C(O)OH or —C(O)OR^{u}.

G305.1. The compound of any one of embodiments G300-G304, where R⁷ is —C(O)OH.

G306. The compound of any one of embodiments G300-G304, where R⁷ is —C(O)N(R^{c}R^{d}) and R^{c} and R^{d} each independently is hydrogen or optionally substituted C1-C4 alkyl.

G307. The compound of embodiment G306, where R^{c} and R^{d} each independently is hydrogen or unsubstituted C1-C4 alkyl; or R^{c} and R^{d} each independently is hydrogen or methyl; or R^{c} and R^{d} each is unsubstituted C1-C4 alkyl; or R^{c} and R^{d} each is methyl.

G308. The compound of embodiment G307, where R⁷ is —C(O)N(CH₃)CH₃, —C(O)N(H)CH₃ or —C(O)NH₂.

G309. The compound of any one of embodiments G300-G308, where R^{u} is an optionally substituted C1-C4 alkyl, butyl, tert-butyl, iso-butyl, propyl, isopropyl, ethyl or methyl.

G310. The compound of any one of embodiments G300-G309, where R^{p}, R^{q}, R^{r} and R^{s} each independently is hydrogen, ethyl or methyl.

G311. The compound of any one of embodiments G300-G310, where X^{1a} is C, X^{2a} is C—R¹⁴, X^{3a} is N, X^{4a} is C—R¹⁶, X^{5a} is N, and X^{6a} is C—R¹⁸.

G312. The compound of embodiment G311, where (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ and R¹⁶ each independently is hydrogen or unsubstituted C1-C4 alkoxy; (iii) R¹⁴ and R¹⁶ each independently is hydrogen or methoxy; (iv) R¹⁴ and R¹⁶ each is methoxy; and/or (v) R¹⁸ is hydrogen.

G313. The compound of embodiment G312, wherein R¹⁰ is

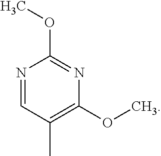

G314. The compound of any one of embodiments G300-G310, where X^{1a} is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused optionally substituted pyrrolyl.

G315. The compound of any one of embodiments G300-G310, where X^{1a} is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused unsubstituted pyrrolyl.

G316. The compound of embodiment G315, wherein one, two or three of R¹⁴, R¹⁷ or R¹⁸ each is hydrogen.

G317. The compound of embodiment G316, where R¹⁰ is

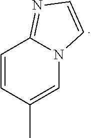

G318. A compound of formula:

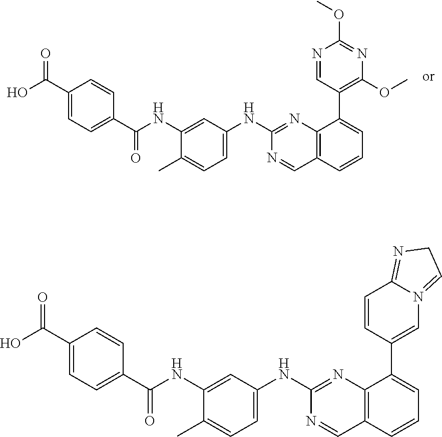

or a pharmaceutically acceptable salt thereof.

G400. A compound of Formula A1-2:

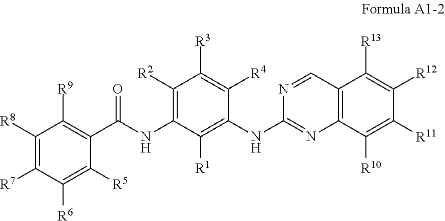

or a pharmaceutically acceptable salt, amide or ester thereof, where:
R¹¹ is of Formula C2:

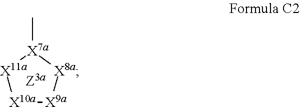

Z^{3a} is heteroaryl; and X^{7a} is C; X^{8a} is N or N—R^{19B}; X^{9a} is N or N—R^{20B}; X^{10a} is C—R²¹; and X^{11a} is C—R²²;
R¹, R², R³, R⁴, R⁵, R⁶, R⁷, R⁸, R⁹, R¹⁰, R¹², R¹³, R²¹ and R²² each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{19B} and R^{20B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH or —C(O)OR^{u};
R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

G401. The compound of embodiment G400, where R¹, R², R³, R⁴, R⁵, R⁶, R⁸, R⁹, R¹⁰, R¹², R¹³, R²¹ and R²² each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

G402. The compound of embodiment G400 or G401, where R¹, R², R³, R⁴, R⁵, R⁶, R⁸, R⁹, R¹⁰, R¹², R¹³, R²¹ and R²² each independently is hydrogen, unsubstituted C1-C4 alkyl, Cl, F, CF₃, CD₃, unsubstituted C1-C4 alkoxy, methoxy, isopropyloxy or dimethylamino;

G403. The compound of any one of embodiments G400-G402, where:
one, two, three or four of R¹, R¹¹, R¹² and R¹³ each is hydrogen;
one, two, three or four of R^{19B}, R^{20B}, R²¹ and R²² each is hydrogen;
one or two of R³ and R⁴ each is hydrogen;
R² is hydrogen; and/or
one, two, three or four of R⁵, R⁶, R⁸ and R⁹ each is hydrogen.

G404. The compound of any one of embodiments G400-G403, where R² is methyl, ethyl, methoxy or ethoxy, and R¹, R³ and R⁴ each is hydrogen.

G405. The compound of any one of embodiments G400-G404, where R⁷ is —C(O)OH or —C(O)OR^{u}.

G405.1. The compound of any one of embodiments G400-G404, where R⁷ is —C(O)OH.

G406. The compound of any one of embodiments G400-G404, where R⁷ is —C(O)N(R^{c}R^{d}) and R^{c} and R^{d} each independently is hydrogen or optionally substituted C1-C4 alkyl.

G407. The compound of embodiment G406, where R^{c} and R^{d} each independently is hydrogen or unsubstituted C1-C4 alkyl; or R^{c} and R^{d} each independently is hydrogen or methyl; or R^{c} and R^{d} each is unsubstituted C1-C4 alkyl; or R^{c} and R^{d} each is methyl.

G408. The compound of embodiment G407, where R⁷ is —C(O)N(CH₃)CH₃, —C(O)N(H)CH₃ or —C(O)NH₂.

G409. The compound of any one of embodiments G400-G408, where R^{u} is an optionally substituted C1-C4 alkyl, butyl, tert-butyl, iso-butyl, propyl, isopropyl, ethyl or methyl.

G410. The compound of any one of embodiments G400-G409, where R^{p}, R^{q}, R^{r} and R^{s} each independently is hydrogen, ethyl or methyl.

G411. The compound of any one of embodiments G400-G410, where R^{19B} and R^{20B} each independently is hydrogen, optionally substituted C1-C4 alkyl, optionally substituted C1-C4 deuteroalkyl, optionally substituted C1-C4 haloalkyl or optionally substituted C1-C4 alkylamino.

G412. The compound of any one of embodiments G400-G411, where: (i) X^{8a} is N and X^{9a} is N—R^{20B}; (ii) R^{19B} and R^{20B} each independently is hydrogen, unsubstituted C1-C4 alkyl, ethyl or methyl; (iii) R²¹ and R²² each independently is hydrogen, unsubstituted C1-C4 alkyl, ethyl or methyl; (iv) R^{19B} and R^{20B} each is hydrogen; and/or (v) R²¹ and R²² each is hydrogen.

G413. The compound of embodiment G412, where R¹¹ is

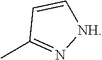

G414. A compound of formula:

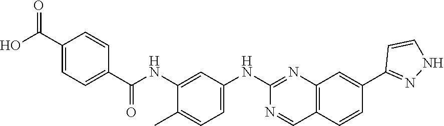 [SMILES: Cc1ccc(Nc2ncc3ccc(-c4cc[nH]n4)cc3n2)cc1NC(=O)c1ccc(C(=O)O)cc1]

or a pharmaceutically acceptable salt thereof.

H1. A compound of any one of embodiments A1-A109, B1-B16, C1-C11, D1-D12, E1-E11, F1-F15, G1-G10, G100-G110, G200-G209, G300-G318 and G400-G414, with the proviso that R¹⁰ and/or optionally one, two or three of R¹¹, R¹² or R¹³, is not: methyl; substituted alkyl or unsubstituted alkyl; methoxy; substituted alkoxy or unsubstituted alkoxy; and/or unsubstituted phenyl.

H2. A compound of any one of embodiments A1-A109, B1-B16, C1-C11, D1-D12, E1-E11, F1-F15, G1-G10, G100-G110, G200-G209, G300-G318 and G400-G414, with the proviso that R¹¹ and R¹² each is not methoxy.

H3. A compound of any one of embodiments A1-A109, B1-B16, C1-C11, D1-D12, E1-E11, F1-F15, G1-G10, G100-G110, G200-G209, G300-G318 and G400-G414, where R₁₀, and/or optionally one, two or three of R¹¹, R¹² or R¹³ is —W—R^{W}, with the proviso that R^{W} is not a phenyl substituted with —C≡CH₃ and W is not amino.

H4. A compound of any one of embodiments A1-A109, B1-B16, C1-C11, D1-D12, E1-E11, F1-F15, G1-G10, G100-G110, G200-G209, G300-G318 and G400-G414, with the proviso that R² and R⁹ do not form a bond.

H5. A compound of any one of embodiments A1-A109, B1-B16, C1-C11, D1-D12, E1-E11, F1-F15, G1-G10, G100-G110, G200-G209, G300-G318 and G400-G414, with the proviso that R² and R³, or optionally R³ and R⁴, are not joined as an imidazolyl group.

H6. A compound of any one of embodiments A1-A109, B1-B16, C1-C11, D1-D12, E1-E11, F1-F15, G1-G10, G100-G110, G200-G209, G300-G318 and G400-G414, with the proviso that R² and R³, or optionally R³ and R⁴, are not joined as: (i) an imidazolyl moiety fused to the phenyl group on which R², R³ and R⁴ are substituents; (ii) an indolyl group; (iii) a five-membered ring; (iv) a five-membered ring fused to the phenyl group on which R², R³ and R⁴ are substituents; (v) unsubstituted heteroaryl containing 5 ring atoms; and/or (vi) substituted heteroaryl containing 5 ring atoms.

H7. A compound of any one of embodiments A1-A109, B1-B16, C1-C11, D1-D12, E1-E11, F1-F15, G1-G10, G100-G110, G200-G209, G300-G318 and G400-G414, with the proviso that (i) the amino group joined by a covalent bond to the carbon ring atom in the quinazolinyl group, which carbon ring atom is positioned between the two nitrogen ring atoms of the quinazolinyl group, and (ii) R¹, do not participate in a five-membered ring, and/or do not join in an indolyl group.

H8. A compound of any one of embodiments A1-A109, B1-B16, C1-C11, D1-D12, E1-E11, F1-F15, G1-G10, G100-G110, G200-G209, G300-G318 and G400-G414, where Y is —NR^{a}R^{b}, —N(R^{b})—CH₂—R^{Y} or —CH₂—N(R^{b})R^{Y}, with the proviso that: (ii) R¹⁶ is not methoxy; (iii) R¹⁵ or R¹⁶ each independently is not

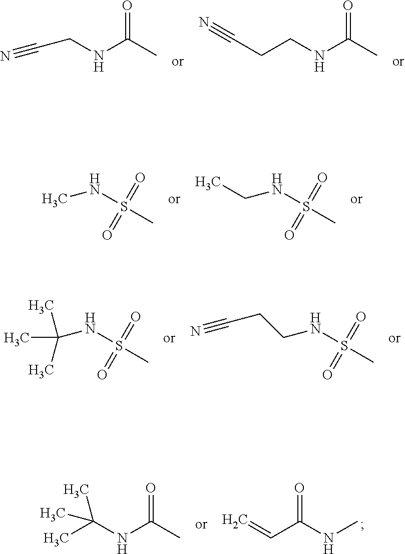

(iv) R¹⁵ or R¹⁶ each independently is not

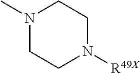

where R^{49X} is methyl, ethyl, methoxy, —C(O)CH₃, —CH₂CH₂OH, —CH₂CH₂OCH₃, —CH₂CH(OH)CH₃, —CH₂C(OH)(CH₃)CH₃, or —CH₂CH₂F; (v) R^{a} and R^{b}, or R^{Y} and R^{b}, are not joined as an unsubstituted piperazinyl, or substituted piperazinyl, or unsubstituted morpholinyl or substituted morpholinyl; (vi) R^{a} and R^{b}, or R^{Y} and R^{b}, are not joined as a group containing an unsubstituted piperazinyl, or substituted piperazinyl, or unsubstituted morpholinyl or substituted morpholinyl; (vii) R^{a} and R^{b}, or R^{Y} and R^{b}, are not joined as an unsubstituted heterocycloalkyl or substituted heterocycloalkyl; (viii) R^{a} and R^{b}, or R^{Y} and R^{b}, are not joined as a group containing an unsubstituted heterocycloalkyl or substituted heterocycloalkyl; (ix) R² is not unsubstituted morpholino or

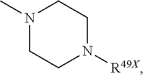

where R^{49X} is methyl, ethyl, methoxy, —C(O)CH₃, —CH₂CH₂OH, —CH₂CH₂OCH₃, —CH₂CH(OH)CH₃, —CH₂C(OH)(CH₃)CH₃, or —CH₂CH₂F; (x) R² is not a substituted alkyl substituted by a group defined in (ix); (xi) R² is not (a) an unsubstituted piperazinyl, or substituted piperazinyl, or unsubstituted morpholinyl or substituted morpholinyl; or (b) an alkyl substituted by a group defined in (xi)(a); (xii) R² is not a group containing an unsubstituted piperazinyl, or substituted piperazinyl, or unsubstituted morpholinyl or substituted morpholinyl; (xiii) R² is not an unsubstituted heterocycloalkyl or substituted heterocycloalkyl; (xiv) R² is not a group containing an unsubstituted heterocycloalkyl or substituted heterocycloalkyl; (xv) R^{b} in —CH₂—N(R^{b})R^{Y} is hydrogen and R^{Y} is not methyl or is not

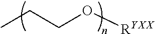

where n is an integer of 1 to 10; and R^{YXX} is hydrogen, optionally substituted alkyl or optionally substituted amidoalkyl; (xvi) for —CH₂—N(R^{b})R^{Y}, R^{b} and R^{Y} each is not methyl; (xvii) for —N(R^{a})R^{b}, R^{a} and R^{b} each is not methyl; (xviii) for —N(R^{a})R^{b}, R^{b} is not unsubstituted C1-C4 alkyl or is not substituted C1-C4 alkyl; and/or (xix) R^{Y} is not an unsubstituted phenyl or substituted phenyl.

H9. A compound of any one of embodiments A1-A109, B1-B16, C1-C11, D1-D12, E1-E11, F1-F15, G1-G10, G100-G110, G200-G209, G300-G318 and G400-G414, with the proviso that: (i) Y or R² each independently is not

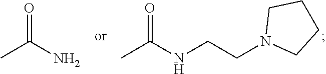

(ii) Y or R² each independently is not —C(O)N(H)R^{50X}, where R^{50X} is hydrogen, unsubstituted alkyl, substituted alkyl, unsubstituted heterocycloalkylalkyl or substituted heterocycloalkyl; (iii) Y or R² each independently is not hydrogen; (iv) Y or R² each independently is not chloro; (v) Y or R² each independently is not fluoro; (vi) Y or R² each independently is not halo; (vii) Y or R² each independently is not methoxy; (viii) Y or R² each independently is not unsubstituted alkoxy; (ix) Y or R² each independently is not cyano; (x) R¹ is not fluoro; (xi) R¹ is not halo; (xii) R⁴ is not fluoro; (xiii) R⁴ is not halo; (xiv) R³ is not fluoro; and/or (xvi) R³ is not halo.

H10. A compound of any one of embodiments A1-A109, B1-B16, C1-C1l, D1-D12, E1-E11, F1-F15, G1-G10, G100-G110, G200-G209, G300-G318 and G400-G414, with the proviso that: (i) R² is not an unsubstituted piperazinyl, or substituted piperazinyl, or unsubstituted morpholinyl or substituted morpholinyl; (ii) R² is not an unsubstituted heterocycloalkyl or substituted heterocycloalkyl; (iii) R² does not contain an unsubstituted heterocycloalkyl or substituted heterocycloalkyl; and/or (iv) R² is hydrogen, unsubstituted C1-C4 alkyl, ethyl or methyl.

H11. A compound of any one of embodiments A1-A109, B1-B16, C1-C11, D1-D12, E1-E11, F1-F15, G1-G10, G100-G110, G200-G209, G300-G318 and G400-G414, where Y is —N(R^{b})C(O)—R^{Y} or —N(R^{b})C(O)—R^{V}—R^{Y} and R^{Y} is an optionally substituted alkenyl, with the proviso that: (i) Y, R¹, R², R³ or R⁴ each independently is not one of the following designated Group A electrophilic groups:

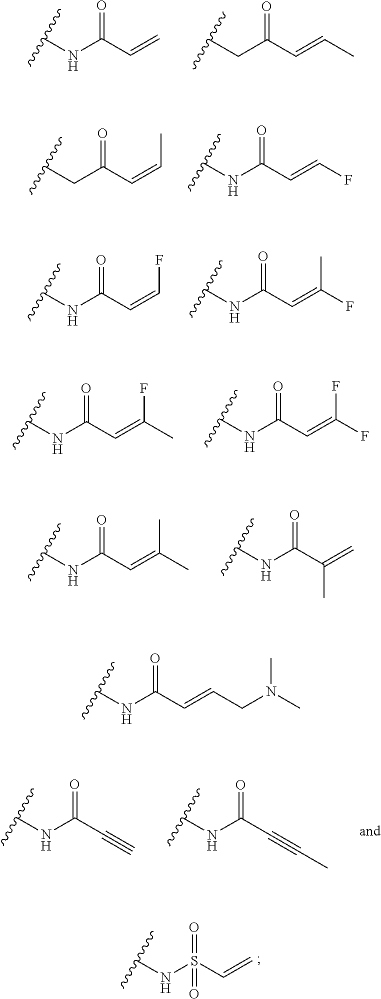

(ii) Y, R¹, R², R³ or R⁴ each independently is not one of the following designated Group B electrophilic groups:

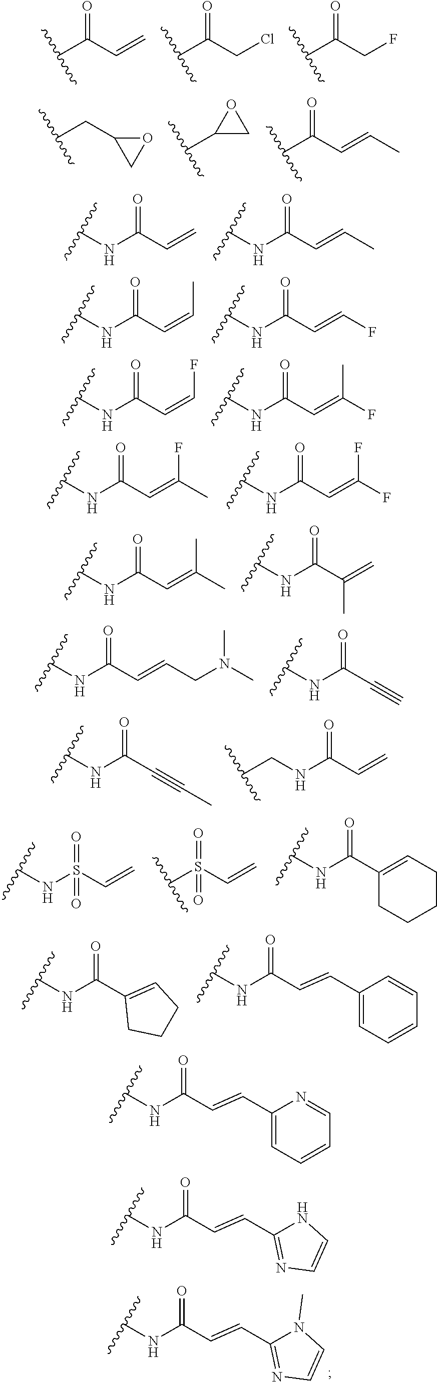

and/or (iii) Y, R¹, R², R³ or R⁴ each independently is not an electrophilic group capable of forming a covalent bond with a cysteine of a protein.

H12. A compound of any one of embodiments A1-A109, B1-B16, C1-C11, D1-D12, E1-E11, F1-F15, G1-G10, G100-G110, G200-G209, G300-G318 and G400-G414, where Y is —N(R^{b})C(O)—R^{Y} or —N(R^{b})C(O)—R^{V}—R^{Y}, and R^{Y} is —CH═CH₂ or Y, R¹, R³ or R⁴ is one of the designated Group A electrophilic groups defined in embodiment H11, one of the designated Group B electrophilic groups defined in embodiment H11, or an electrophilic group capable of forming a covalent bond with a cysteine of a protein, with the proviso that: (i) R² is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 mercaptoalkyl, R^{c}C(O)N(R^{d})—, —C(O)N(R^{c}R^{d}), —C(O)R^{z}, —C(O)OH, —C(O)OR^{u}, —B(OH)₂, hydroxy, halo, nitro or cyano, where R^{c}, R^{d} and R^{u} each independently is hydrogen or optionally substituted C1-C6 alkyl, optionally substituted C1-C4 alkyl, butyl, tert-butyl, iso-butyl, propyl, iso-propyl, ethyl or methyl and where R^{u} is an optionally substituted C1-C6 alkyl, optionally substituted C1-C4 alkyl, butyl, tert-butyl, iso-butyl, propyl, iso-propyl, ethyl or methyl; (ii) R² is hydrogen, optionally substituted C1-C4 alkyl, optionally substituted C1-C4 deuteroalkyl, optionally substituted C1-C4 haloalkyl, optionally substituted C1-C4 alkylamino or halo; (iii) R² is hydrogen, unsubstituted C1-C4 alkyl, unsubstituted C1-C4 deuteroalkyl, unsubstituted C1-C4 haloalkyl or halo; (iv) R² is unsubstituted C1-C4 alkoxy, ethoxy or methoxy; and/or (v) R² is unsubstituted C1-C4 alkyl or methyl.

H13. A compound of any one of embodiments A1-A109, B1-B16, C1-C11, D1-D12, E1-E11, F1-F15, G1-G10, G100-G110, G200-G209, G300-G318 and G400-G414, where Y is —N(R^{b})C(O)—R^{Y} or —N(R^{b})C(O)—R^{V}—R^{Y}, and R^{Y} is —CH═CH₂ or Y, R¹, R³ or R⁴ is one of the designated Group A electrophilic groups defined in embodiment H11, one of the designated Group B electrophilic groups defined in embodiment H11, or an electrophilic group capable of forming a covalent bond with a cysteine of a protein, with the proviso that: (i) R² is not —NR^{a}R^{b}, where R^{a} and R^{b} are joined as an unsubstituted morpholino, substituted piperazinyl or substituted azetidinyl; (ii) R² is not —NR^{a}R^{b}, where R^{a} and R^{b} are joined as a substituted heterocycloalkyl or unsubstituted heterocycloalkyl; (iii) R² is not —NR^{a}R^{b}, where R^{a} is —CH₂CH₂N(CH₃)CH₃ and R^{b} is hydrogen or methyl; (iv) R² is not —NR^{a}R^{b}, where R^{a} is unsubstituted aminoalkyl or substituted aminoalkyl and R^{b} is hydrogen or unsubstituted alkyl; (v) R² is not —O—R^{51X}, where R^{51X} is —CH₂CH₂N(CH₃)CH₃, —CH₂CH₂OCH₃, substituted piperidinyl, substituted pyrrolidinyl or substituted azetidinyl; (vi) R² is not —O—R^{51X}, where R^{51X} is unsubstituted aminoalkyl or substituted aminoalkyl, unsubstituted alkoxyalkyl or substituted alkoxyalkyl, unsubstituted heterocycloalkyl or substituted heterocycloalkyl; (vii) R⁴ is not methoxy or fluoro; (viii) R⁴ is not unsubstituted alkoxy or halo; (ix) R¹⁷ and R¹⁸ are not fluoro or chloro; and/or (x) R¹⁷ and R¹⁸ are not halo.

H14. A compound of any one of embodiments A1-A109, B1-B16, C1-C11, D1-D12, E1-E11, F1-F15, G1-G10, G100-G110, G200-G209, G300-G318 and G400-G414, where at least one of R¹⁰, R¹¹, R¹² or R¹³ is R^{W} or —W—R^{W}, with the proviso that R¹⁴, R¹⁵, R¹⁶, R¹⁷, R¹⁸, R^{14A}, R^{15A}, R^{16A}, R^{17A}, R^{18A}, R^{aB}, R¹⁹, R^{19A}, R²⁰, R²⁰, R²¹, R^{21A}, R²², R^{22A}, R^{14B}, R^{15B}, R^{16B}, R^{17B}, R^{18B}, R^{19B}, R^{20B}, R^{21B} and R^{22B} each independently is (i) not

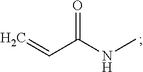

(ii) not one of the designated Group A electrophilic groups defined in embodiment H11; (iii) not one of the designated Group B electrophilic groups defined in embodiment H11, and/or (iv) not an electrophilic group capable of forming a covalent bond with a cysteine of a protein.

J1. A compound disclosed in Table A, or a pharmaceutically acceptable salt, ester or amide thereof.

J2. A compound disclosed in Table A, or a pharmaceutically acceptable salt thereof.

J3. The compound of embodiment J1 or J2, which is a hydrochloride salt.

J4. A composition, comprising a compound of any one of embodiments A1-A109, B1-B16, C1-C11, D1-D12, E1-E11, F1-F15, G1-G10, G100-G110, G200-G209, G300-G318, G400-G414, H1-H14 and J1-J3.

J5. The composition of embodiment J4, where the compound is capable of inhibiting an activity of a protein kinase (PK).

J6. The composition of embodiment J5, where the PK activity is a PK binding activity and/or a PK catalytic activity.

J7. The composition of embodiment J5 or J6, where the compound is capable of inhibiting an activity of two or more PKs.

J7.1. The composition of any one of embodiments J5-J7 where the compound is capable of effectively inhibiting, moderately inhibiting and/or selectively inhibiting a PK activity.

J8. The composition of any one of embodiments J4-J7.1, which is a pharmaceutical composition comprising one or more pharmaceutically acceptable excipients.

J9. The composition of embodiment J8, for topical administration, oral administration or administration by injection or infusion.

K1. Use of a compound or composition of any one of embodiments A1-A109, B1-B16, C1-C11, D1-D12, E1-E11, F1-F15, G1-G10, G100-G110, G200-G209, G300-G318, G400-G414, H1-H14 and J1-J9 for inhibiting a protein kinase (PK).

K2. The use of embodiment K1, for effectively inhibiting or moderately inhibiting a PK.

K3. Use of a composition comprising a compound, the compound comprising a quinazolinyl group and an amine-linked phenyl group for inhibiting two or more of, or three or more of, or four or more of, or five or more of, or six or more of, or seven or more of, or eight or more of, or nine or more of, or ten or more of, or eleven or more of, or twelve or more of, or thirteen or more of, or each of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and a PTK family PK.

K4. Use of a composition comprising a compound comprising a quinazolinyl group and an amine-linked phenyl group for selectively inhibiting a JAK2 PK.

K5. The use of embodiment K4, for effectively inhibiting JAK2(wt) PK and not effectively inhibiting JAK1(wt) PK.

K6. The use of embodiment K4 or K5, for inhibiting a JAK3(wt) PK, optionally for effectively inhibiting a JAK3(wt) PK, and optionally for moderately inhibiting a JAK3(wt) PK.

K7. The use of any one of embodiments K4-K6, for effectively inhibiting and/or moderately inhibiting two or more of, or three or more of, or four or more of, or five or more of, or six or more of, or seven or more of, or eight or more of, or nine or more of, or ten or more of, or eleven or more of, or twelve or more of, or thirteen or more of, or each of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and a PTK family PK.

K8. The use of any one of embodiments K3-K7, where the composition comprises a compound of, or is a composition of, any one of embodiments A1-A109, B1-B16, C1-C11, D1-D12, E1-E11, F1-F15, G1-G10, G100-G110, G200-G209, G300-G318, G400-G414, H1-H14 and J1-J9.

K9. The use of any one of embodiments K1-K8, for effectively inhibiting two or more of, or three or more of, or four or more of, or five or more of, or six or more of, or seven or more of, or eight or more of, or nine or more of, or ten or more of, or eleven or more of, or twelve or more of, or thirteen or more of, or each of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

K9.1. The use of embodiment K9, for moderately inhibiting two or more of, or three or more of, or four or more of, or five or more of, or six or more of, or seven or more of, or eight or more of, or nine or more of, or ten or more of, or eleven or more of, or twelve or more of, or thirteen or more of, or each of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

K10. The use of embodiment K9 or K9.1, for not effectively inhibiting and/or for not moderately inhibiting one or more of, or two or more of, or three or more of, or four or more of, or five or more of, or six or more of, or seven or more of, or eight or more of, or nine or more of, or ten or more of, or eleven or more of, or twelve or more of, or thirteen or more of: an ABL family PK, a BTK family PK, a AURK family PK, a JAK family PK, a TRK family PK, a RET PK, an EPH family PK, a TNK family PK, a PLK family PK, an IRAK family PK, a SRC family PK, a LCK family PK, a DDR family PK, and/or a PTK family PK.

K11. The use of any one of embodiments K1-K10, for inhibiting an ABL family PK, optionally inhibiting ABL1(wt), optionally inhibiting one or more ABL1 variants, optionally inhibiting ABL2(wt), and/or optionally inhibiting one or more ABL2 variants; where the one or more ABL1 variants optionally comprise one or more of the following amino acid substitutions relative to ABL1(wt): T315I, G250E, Q252H, Y253F, E255K, F317L, M351T and H396P.

K12. The use of any one of embodiments K1-K11, for inhibiting a BTK family PK, and optionally inhibiting one or more of: BTK(wt), one or more BTK variants, BMX(wt), one or more BMX variants, ITK(wt), one or more ITK variants, TEC(wt), one or more TEC variants, TXK(wt), and one or more TXK variants.

K13. The use of embodiment K12, for inhibiting TXK(wt) and/or one or more TXK variants.

K14. The use of embodiment K13, where the composition comprises a compound of any one of embodiments G200-G209 and H1-H14.

K15. The use of any one of embodiments K1-K14, for inhibiting a AURK family PK, and optionally inhibiting one or more of: AURKA(wt), one or more AURKA variants; AURKB(wt), one or more AURKB variants, AURKC(wt), and one or more AURKC variants.

K16. The use of any one of embodiments K1-K15, for inhibiting a JAK family PK, and optionally inhibiting one or more of: JAK (wt), one or more JAK1 variants, JAK2(wt), one or more JAK2 variants, JAK3(wt), one or more JAK3 variants, a TYK family PK; a TYK2(wt) and one or more TYK2 variants; where the one or more JAK2 PK variants optionally comprise the amino acid substitution V617F relative to JAK2(wt).

K17. The use of any one of embodiments K1-K16, for inhibiting JAK2(wt) and optionally one or more JAK2 variants.

K18. The use of embodiment K17, for inhibiting JAK3(wt) and optionally one or more JAK3 variants.

K19. The use of embodiment K17 or K18, for not effectively inhibiting JAK (wt) and optionally not moderately inhibiting JAK (wt).

K20. The use of any one of embodiments K17-K19, for: (i) not effectively inhibiting one or more JAK1 variants; (ii) not effectively inhibiting ABL1(wt) and/or ABL1(T315I); (iii) mildly inhibiting ABL1(wt) and/or ABL1(T315I); (iv) ineffectively inhibiting ABL1(wt) and/or ABL1(T315I); (v) effectively inhibiting ABL1(wt) and effectively inhibiting ABL1(T315I); (vi) moderately inhibiting ABL1(wt); and/or (vii) not effectively inhibiting, and/or optionally not moderately inhibiting, one or more of, or two or more of, or three or more of, or four or more of, or five or more of, or six or more of, or seven or more of, or eight or more of, or nine or more of, or ten or more of, or eleven or more of, or twelve or more of, or each of: an ABL family PK, a BTK family PK, a AURK family PK, a TRK family PK, a RET PK, an EPH family PK, a TNK family PK, a PLK family PK, an IRAK family PK, a SRC family PK, a LCK family PK, a DDR family PK, and/or a PTK family PK.

K20. The use of any one of embodiments K17-K19, where the composition comprises a compound of any one of embodiments C1-C11, D1-D12, E1-E11, G1-G10, G100-G110, G200-G209 and H1-H14.

K21. The use of any one of embodiments K17-K20, for inhibiting JAK2(wt) and a JAK2 variant comprising the V617F substitution.

K22. The use of embodiment K21, where the composition comprises a compound of any one of embodiments D1-D12, G100-G110 and H1-H14.

K23. The use of any one of embodiments K16-K22, for inhibiting, optionally effectively inhibiting or optionally moderately inhibiting, a TYK family PK.

K24. The use of embodiment K21, for inhibiting, optionally effectively inhibiting or optionally moderately inhibiting TYK2(wt).

K25. The use of embodiment K23 or K24, for not effectively inhibiting one or more of, or two or more of, or each of JAK (wt), JAK2(wt) and JAK3(wt), and optionally for not moderately inhibiting one or more of, or two or more of, or each of JAK (wt), JAK2(wt) and/or JAK3(wt).

K26. The use of any one of embodiments, K23-K26, where the composition comprises a compound of any one of embodiments G400-G414 and H1-H14.

K27. The use of any one of embodiments K1-K26, for inhibiting a TRK family PK, and optionally inhibiting one or more of: TRKA(wt), one or more TRKA variants, TRKB(wt), one or more TRKB variants, TRKC(wt), one or more TRKC variants, ROS1(wt) and one or more ROS1 variants.

K28. The use of any one of embodiments K1-K27, for inhibiting a RET family PK, and optionally: inhibiting RET(wt) and/or one or more RET variants, where the one or more RET variants optionally comprise one or more of the following amino acid substitutions relative to RET(wt): A883F, G691S, M918T, S891A, V804E, V804L, V804M and Y791F.

K29. The use of any one of embodiments K1-K28, for inhibiting an EPH family PK, and optionally inhibiting one or more of: EPHA1(wt), one or more EPHA1 variants, EPHA2(wt), one or more EPHA2 variants, EPHB1(wt), and one or more EPHB1 variants.

K30. The use of any one of embodiments K1-K29, for inhibiting a TNK family PK, and optionally: inhibiting TNK1(wt) and/or one or more TNK1 variants.

K31. The use of any one of embodiments K1-K30, for inhibiting a PLK family PK, and optionally: inhibiting PLK4(wt) and/or one or more PLK4 variants.

K32. The use of any one of embodiments K1-K31, for inhibiting an IRAK family PK, and optionally: inhibiting IRAK3(wt) and/or one or more IRAK3 variants.

K33. The use of embodiment K31 or K32, where the composition comprises a compound of any one of embodiments G300-G318 and H1-H14.

K34. The use of any one of embodiments K1-K33, for inhibiting one or more, or two or more, variants of a PK.

K35. The use of embodiment K34, for inhibiting one or more of the following PK variants: (i) an ABL1 variant optionally comprising one or more of the following amino acid substitutions relative to ABL1(wt): T315I, G250E, Q252H, Y253F, E255K, F317L, M351T and H396P; (ii) a JAK2 PK variant optionally comprising the amino acid substitution V617F relative to JAK2(wt); and/or (iii) a RET variant optionally comprising one or more of the following amino acid substitutions relative to RET(wt): A883F, G691S, M918T, S891A, V804E, V804L, V804M and Y791F.

K36. The use of any one of embodiments K1-K35, for inhibiting two or more PKs including: (i) an ABL family PK and a BTK family PK; (ii) an ABL family PK and a AURK family PK; and (iii) an ABL family PK and a JAK family PK.

K37. The use of any one of embodiments K1-K36, for inhibiting three or more PKs including an ABL family PK, a BTK family PK and a AURK family PK.

K38. The use of any one of embodiments K1-K37, for inhibiting four or more PKs including an ABL family PK, a BTK family PK, an AURK family PK, and a JAK family PK; and optionally including ABL1(wt), and optionally an ABL1 variant containing a T315I substitution, in the ABL family; BTK(wt) PK in the BTK family; AURKA(wt), AURKB(wt) and AURKC(wt) in the AURK family; and JAK2(wt), and optionally a JAK2 variant containing a V617F substitution, in the JAK family.

K39. The use of any one of embodiments K1-K38, for inhibiting five or more PKs including an ABL family PK, a BTK family PK, an AURK family PK, a JAK family PK and a TRK family PK.

K40. The use of any one of embodiments K1-K39, for inhibiting six or more PKs including an ABL family PK, a BTK family PK, an AURK family PK, a JAK family PK, a TRK family PK and a RET family PK.

K41. The use of any one of embodiments K1-K40, for effectively inhibiting ABL1, ABL1-T315I, AURKA, and JAK2.

K41.1. The use of embodiment K41, for effectively inhibiting or moderately inhibiting BTK.

K41.2. The use of embodiment K41 or K41.1, for effectively inhibiting or moderately inhibiting JAK3.

K41.3. The use of any one of embodiments K41-K41.2, for effectively inhibiting or moderately inhibiting JAK1.

K41.4. The use of any one of embodiments K41-K41.2, for mildly inhibiting or ineffectively inhibiting JAK1.

K42. The use of any one of embodiments K41-K41.4, for effectively inhibiting or moderately inhibiting one or more or all of TYK2, A TRK family PK, ROS1, TXK, an EPH family PK, IRAK3, PLK4, TNK1, RET and JAK2-V617F.

K43. The use of any one of embodiments K41-K42, for effectively inhibiting or moderately inhibiting one or more or all of TRKA, TRKB, TRKC, ROS1, EPHA1 and EPHB1.

K43.1. The use of any one of embodiments K41-K43, for effectively inhibiting or moderately inhibiting a SRC family PK and/or a DDR family PK.

K43.2. The use of embodiment K43.1, where the DDR family PK is DDR2 PK and optionally a DDR2 variant PK.

K43.3. The use of embodiment K43.2, where the DDR2 variant PK contains a T654M and/or N456S amino acid substitution.

K43.4. The use of any one of embodiments K43.1—K43.3, where the SRC family PK is SRC(wt) or SRC-N1 (wt).

K43.5. The use of any one of embodiments K43.1—K43.4, for (i) effectively inhibiting SRC(wt), SRC-N1 (wt), and a DDR2 variant, optionally a DDR2-T654M variant; or (ii) mildly inhibiting SRC(wt), moderately inhibiting SRC-N1 (wt), and effectively inhibiting a DDR2 variant, optionally a DDR2-T654M variant.

K43.6. The use of any one of embodiments K41-K43.5, for effectively inhibiting or moderately inhibiting ABL2.

K43.7. The use of any one of embodiments K41-K43.6, for effectively inhibiting or moderately inhibiting PTK2B.

K43.8. The use of any one of embodiments K41-K43.7, for effectively inhibiting or moderately inhibiting ABL1, ABL1-T315I, ABL2, TYK2, JAK2, TRKC and PTK2B.

K43.9. The use of any one of embodiments K41-K43.8, for effectively inhibiting or moderately inhibiting ABL1, ABL1-T315I, BTK, AURKA, JAK2, NTRK3, PTK2B, TYK2 and ABL2.

K43.10. The use of embodiment K43.9, where the NTRK3 is a ETV6-NTRK3 fusion.

K43.11. the use of any one of embodiments K41-K43.10, for effectively inhibiting or moderately inhibiting a LCK family PK, and optionally LCK(wt).

K44. The use of any one of embodiments K1-K43.11, where the composition comprises a compound of any one of embodiments A1-A109 and H1-H14.

K45. The use of any one of embodiments K1-K44, where the composition comprises a compound of any one of embodiments B1-B16, F1-F15 and H1-H14.

K46. The use of any one of embodiments K1-K40, for effectively inhibiting JAK2, JAK3, JAK2-V617F and mildly inhibiting JAK1.

K47. The use of embodiment K46, for effectively inhibiting or moderately inhibiting one or more or all of ABL1, TRKA, TRKB, TRKC, ROS1, AURKA, IRAK3, TNK1.

K48. The use of embodiment K46 or K47, where the composition comprises a compound of any one of embodiments G1-G10 and H1-H14.

K49. The use of any one of embodiments K1-K40, for effectively inhibiting JAK2, ineffectively inhibiting or mildly inhibiting JAK1, and optionally: moderately inhibiting JAK3 and/or mildly inhibiting JAK2-V617F.

K50. The use of embodiment K49, for effectively inhibiting or moderately inhibiting one or more or all of TRKB, TRKC, AURKA, IRAK3 and PLK4.

K51. The use of embodiment K49 or K50, where the composition comprises a compound of any one of embodiments E1-E11 and H1-H14.

K52. The use of any one of embodiments K1-K40, for effectively inhibiting JAK2, ineffectively inhibiting or mildly inhibiting JAK1, and optionally: moderately inhibiting JAK3 and/or JAK2-V617F.

K53. The use of embodiment K52, for effectively inhibiting or moderately inhibiting one or more or all of TXK, JAK2-V617F, TRKB, TRKC, ROS1, AURKA, IRAK3 and TNK1.

K54. The use of embodiment K52 or K53, where the composition comprises a compound of any one of embodiments C1-C11 and H1-H14.

K55. The use of any one of embodiments K1-K40, for effectively inhibiting JAK2, ineffectively inhibiting or mildly inhibiting JAK1, and optionally: effectively inhibiting JAK3 and/or JAK2-V617F.

K56. The use of embodiment K55, where the composition comprises a compound of any one of embodiments D1-D12 and H1-H14.

K57. The use of any one of embodiments K1-K40, for effectively inhibiting JAK2, moderately inhibiting JAK1, and optionally: moderately inhibiting JAK3 and/or moderately inhibiting or effectively inhibiting JAK2-V617F.

K58. The use of embodiment K57, where the composition comprises a compound of any one of embodiments G100-G110 and H1-H14.

K59. The use of any one of embodiments K1-K40, for effectively inhibiting or moderately inhibiting TYK2.

K60. The use of embodiment K59, for effectively inhibiting or moderately inhibiting JAK3.

K61. The use of embodiment K59 or K60, for mildly inhibiting or ineffectively inhibiting JAK1 and/or JAK2.

K62. The use of any one of embodiments K59-K61, for effectively inhibiting or moderately inhibiting one or more or all of ABL1-T315I, AURKA, PLK4 and IRAK3.

K63. The use of embodiment K57, where the composition comprises a compound of any one of embodiments G400-G414 and H1-H14.

K64. The use of any one of embodiments K1-K40, for effectively inhibiting or moderately inhibiting TXK.

K65. The use of embodiment K64, for effectively inhibiting JAK2, for ineffectively inhibiting or mildly inhibiting JAK1, and optionally: for effectively inhibiting or moderately inhibiting JAK3 and/or JAK2-V617F.

K66. The use of embodiment K64 or 65, where the composition comprises a compound of any one of embodiments G200-G209 and H1-H14.

K67. The use of any one of embodiments K1-K40, for effectively inhibiting or moderately inhibiting IRAK3.

K68. The use of embodiment K67, where the composition comprises a compound of any one of embodiments G300-G318 and H1-H14.

K69. The use of any one of embodiments K1-K40, for effectively inhibiting or moderately inhibiting PLK4.

K70. The use of embodiment K69, where the composition comprises a compound of any one of embodiments G300-G318 and H1-H14.

K70.1. The use of any one of embodiments K41-K70, wherein the effectively inhibiting or moderately inhibiting is effectively inhibiting.

K71. The use of any one of embodiments K1-K70.1, for inhibiting a PK activity, optionally for inhibiting a PK binding activity, and/or optionally for inhibiting a PK phosphorylation activity.

K72. The use of any one of embodiments K1-K71, where the PK is a Homo sapiens PK.

K73. The use of any one of embodiments K1-K72, which is in vitro or ex vivo.

K74. The use of any one of embodiments K1-K72, which is in vivo.

K75. The use of embodiment K73 or K74, which is in cells, an organ and/or a tissue.

K76. The use of embodiment K74 or K75, which is in a subject.

L1. Use of a compound or composition of any one of embodiments A1-A109, B1-B16, C1-C11, D1-D12, E1-E11, F1-F15, G1-G10, G100-G110, G200-G209, G300-G318, G400-G414, H1-H14 and J1-J9, for treatment of a medical condition or for preparation of a medicament for treatment of a medical condition.

L2. The use of any one of embodiments K1-K76 for treatment of a medical condition or for preparation of a medicament for treatment of a medical condition.

L3. The use of embodiment L1 or L2, where the medical condition is associated with a protein kinase (PK) aberration.

L4. The use of embodiment L3, where the medical condition is associated with dysregulation of a PK and/or is associated with a PK modification.

L5. The use of embodiment L3 or L4, where the PK is one or more of, or two or more of, or three or more of, or four or more of, or five or more of, or six or more of, or seven or more of, or eight or more of, or nine or more of, or ten or more of, or eleven or more of, or twelve or more of, or thirteen or more of, or each of: an ABL family PK, a BTK family PK, a AURK family PK, a JAK family PK, a TRK family PK, a RET PK, an EPH family PK, a TNK family PK, a PLK family PK, an IRAK family PK, SRC family PK, DDR family PK, and/or PTK family PK.

L6. The use of any one of embodiments L1-L5, where the medical condition is a cancer.

L7. The use of embodiment L6, where the cancer is a lymphoma, thymoma, leukemia, carcinoma, glioma, sarcoma, liposarcoma, adenocarcinoma, adenosarcoma or adenoma.

L8. The use of embodiment L6, where the cancer is a cancer occurring in one or more of blood, lymph node, thymus, thyroid, breast, heart, lung, small intestine, colon, rectum, spleen, kidney, bladder, head, neck, esophagus, ovary, prostate, brain, pancreas, skin, bone, bone marrow, uterus, testicles, cervix and liver.

L9. The use of embodiment L8, where the cancer is a blood cancer.

L10. The use of embodiment L9, where the blood cancer is a leukemia, myelodysplastic syndrome (MDS), myelofibrosis, polycythemia vera or essential thrombocythemia.

L11. The use of embodiment L10, where the leukemia is acute myeloid leukemia (AML), chronic myeloid leukemia (CML) or acute lymphoblastic leukemia (ALL).

L11.1. The use of embodiment L11, where the CML is chronic phase CML (CML-CP), acute phase CML (CML-AP) and blast phase CML (CML-BP).

L11.2. The use of embodiment L11, where the ALL is a relapsed and/or refractory ALL (R/R ALL).

L11.3. The use of embodiment L11 or L11.2, where the ALL is Philadelphia chromosome-positive ALL, Philadelphia chromosome-positive-like-ALL, B-cell acute lymphoblastic leukemia (B-ALL) or T-cell acute lymphoblastic leukemia (T-ALL).

L11.4. The use of any one of embodiments L11-L11.3, where the cancer is ALL and optionally the ALL is Philadelphia chromosome-positive ALL.

L11.5. The use of any one of embodiments L9-L11.4, where the compound effectively inhibits or moderately inhibits ABL1 and optionally ABL1-T315I.

L11.6. The use of any one of embodiments L9-L11.5, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L11.6. The use of any one of embodiments L11-L11.3, where the cancer is ALL and optionally the ALL is Philadelphia chromosome-positive-like ALL.

L11.7 The use of embodiment L11.6, where the cancer is associated with an ABL1 PK aberration.

L11.8. The use of embodiment L11.6 or L11.7, where the compound effectively inhibits or moderately inhibits ABL1 and optionally ABL1-T315I.

L11.9. The use of any one of embodiments L11.6-L11.8, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits ABL2, TYK2, TRKC and PTK2B; (iv) effectively inhibits or moderately inhibits JAK1; (v) mildly inhibits or ineffectively inhibits JAK1; (vi) effectively inhibits or moderately inhibits one or more of all of TYK2, a TRK family PK, ROS1, TXK, an EPH family PK, IRAK3, PLK4, TNK1, RET and JAK2-V617F; or (vii) a combination of (i) and (ii); (i) and (iii); (i) and (iv); (i) and (v); (i) and (vi); (iii) and (iv); (iii) and (v); (i), (ii) and (iii); (i), (ii), (iii) and (vi); (i), (ii), (iii) and (iv); (i), (ii), (iii) and (v); (i), (ii), (iii), (iv) and (vi); or (i), (ii), (iii), (v) and (vi).

L11.10. The use of any one of embodiments L11-11.3, where the cancer is an ALL, optionally is a B-ALL, optionally is a TCF3-HLF-positive B-ALL, or optionally is a TCF3-HLF-positive acute B-ALL.

L11.11. The use of embodiment L11.10, where the cancer is associated with an AURK family PK aberration.

L11.12. The use of embodiment L11.10 or L11.11, where the compound effectively inhibits or moderately inhibits an AURKA family PK, optionally AURKA(wt) and/or optionally an AURKA variant.

L11.13. The use of embodiment L11.12, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L11.14. The use of any one of any one of embodiments L11-L11.3, where the cancer is an ALL, optionally is T-ALL, or optionally is a R/R T-ALL.

L11.15. The use of embodiment L11.14, where the cancer is associated with a LCK aberration.

L11.16. The use of embodiment L11.14 or L11.15, where the compound (i) effectively inhibits or moderately inhibits a LCK family PK, optionally LCK(wt), and/or optionally an LCK variant.

L11.17. The use of embodiment L11.16, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L12. The use of embodiments L11 or L11.1, where the cancer is a CML, and optionally CML in a subject having no available protein kinase inhibitor options.

L12.1. The use of any one of embodiments L1-L8, where the solid cancer or ASCVD is associated with a LCK family PK aberration.

L12.2. The use of any one of embodiments L1-L8 and L12.1, where the cancer is a solid cancer, and optionally is a colon cancer, lung cancer, lung carcinoma, breast cancer, blood cancer or ovarian cancer or the medical condition is atherosclerotic coronary vascular disease (ASCVD).

L12.3. The use of embodiment L12.2, where the blood cancer is peripheral T-cell lymphoma (PTCL), PTCL-NOS, PTCL associated with a KHDRBS1-LCK gene fusion and PTCL-NOS associated with a KHDRBS1-LCK gene fusion.

L12.4. The use of any one of embodiments L12.1-L12.3, where the compound (i) effectively inhibits or moderately inhibits a LCK family PK, optionally LCK(wt) and/or optionally a LCK variant.

L12.5. The use of embodiment L12.4, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L12.6. The use of any one of embodiments L1-L8, where the cancer is associated with a DDR family PK aberration and/or a SRC family PK aberration.

L12.7. The use of any one of embodiments L1-L8 and L12.6, where the cancer is a lung cancer.

L12.8. The use of embodiment L12.7, where the lung cancer is non-small cell lung cancer (NSCLC).

L12.9. The use of any one of embodiments L12.6-L12.8, where the DDR family PK is a DDR2(wt) PK and/or a DDR2 variant PK.

L12.10. The use of embodiment L12.9, where the DDR2 variant PK contains a N456S and/or T654M amino acid substitution.

L12.11. The use of any one of embodiments L12.6-L12.10, where the compound (i) effectively inhibits or moderately inhibits SRC(wt), SCR—N1 (wt), DDR2(wt) and/or a DDR2 variant containing a T654M and/or N456S amino acid substitution.

L12.12. The use of any one of embodiments L12.6-L12.11, where the compound (i) effectively inhibits SRC(wt), SRC-N1 (wt), and a DDR2 variant, optionally a DDR2-T654M variant; or (ii) where the compound mildly inhibits SRC(wt), moderately inhibits SRC-N1 (wt), and effectively inhibits a DDR2 variant, optionally a DDR2-T654M variant.

L12.12. The use of embodiment L12.11, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L13. The use of any one of embodiments L1-L12.12, where the medical condition is treated in a subject identified as having an ABL1-T315I variant.

L13.1. The use of any one of embodiments L1-L13, where the medical condition is treated in a subject not identified as having an ABL1-T315I variant or identified as not having an ABL1-T315I variant, and is resistant and/or intolerant to at least two PK inhibitors.

L14. The use of any one of embodiments L6-L8, where the cancer is associated with a TRK family PK aberration, optionally a TRKA family PK aberration, optionally a TRKB family PK aberration and/or optionally a TRKC family PK aberration.

L15. The use of embodiment L14, where the cancer is associated with a TRK family PK fusion, and optionally where the TRK portion of the fusion does not contain a known acquired resistance mutation.

L16. The use of embodiment L14 or L15, where the cancer is a solid tumor associated with a TRK family PK fusion.

L16.1. The use of any one of embodiments L14-L16, where the compound effectively inhibits or moderately inhibits a TRK family PK, and optionally a TRKA family PK, a TRKB family PK and/or a TRKC family PK.

L16.2. The use of embodiment L16.1, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L17. The use of any one of embodiments L1-L11, where the cancer is associated with a ROS family PK aberration.

L18. The use of any one of embodiments L6-L8 and L17, where the cancer is a lung cancer associated with a ROS1 aberration, and optionally a stage IV lung cancer.

L18.1. The use of embodiment L17 or L18, where the compound effectively inhibits or moderately inhibits a ROS family PK or optionally a ROS1 family PK.

L18.2. The use of embodiment L18.1, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L19. The use of any one of embodiments L6-L8, where the cancer is associated with an EPH family PK aberration, and optionally an EPHA1 family PK aberration, EPHA2 family PK aberration, EPHA5 family PK aberration, EPHA8 family PK aberration, EPHB1 family PK aberration and/or EPHB2 family PK aberration.

L20. The use of any one of embodiments L6-L8 and L19, where the cancer is a breast cancer, lung cancer, brain cancer, spinal cancer, gastric cancer, or skin cancer, and optionally a solid tumor cancer.

L21. The use of embodiment L20, where the lung cancer is non-small cell lung cancer (NSCLC); the skin cancer is myeloma; and the brain cancer or spinal cancer is a glioblastoma.

L21.1. The use of any one of embodiments L19-L21, where the compound effectively inhibits or moderately inhibits an EPH family PK, optionally an EPHA1 family PK, optionally a EPHA2 family PK, optionally a EPHA5 family PK, optionally a EPHA8 family PK, optionally a EPHB1 family PK and/or optionally a EPHB2 family PK.

L21.2. The use of embodiment L21.1, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L22. The use of any one of embodiments L6-L8, where the cancer is associated with a TNK family PK aberration, and optionally with a TNK1 family PK aberration.

L23. The use of embodiment L22, where the cancer is a cancer deficient in LKB1.

L23.1. The use of embodiment L22 or L23, where the cancer is a cancer associated with a TNK1 variant to which binding of a 14-3-3 protein is weaker than to TNK1(wt), and optionally is Hodgkin lymphoma.

L23.2. The use of any one of embodiments L22-L23.1, where the compound effectively inhibits or moderately inhibits a TNK family PK and optionally a TNK1 family PK.

L23.3. The use of embodiment L23.2, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L24. The use of any one of embodiments L6-L8, where the cancer is associated with a RET family PK aberration.

L25. The use of any one of embodiments L6-L9 and L24, where the cancer is a lung cancer, a thyroid cancer, a colon cancer or a skin cancer.

L26. The use of embodiment L25, where: the lung cancer is a lung nodule cancer, non-small cell lung cancer (NSCLC), small cell lung cancer, lung adenocarcinoma or mesothelioma; the thyroid cancer is medullary thyroid cancer (MTC), papillary thyroid cancer (PTC) or thyroid gland medullary carcinoma; the colon cancer is colon adenocarcinoma; and/or the skin cancer is melanoma or cutaneous melanoma.

L26.1. The use of any one of embodiments L24-L26, where the compound effectively inhibits or moderately inhibits a RET family PK.

L27. The use of embodiment L26, where the compound (i) effectively inhibits or moderately inhibits ABL1 and optionally ABL1-T315I; (ii) effectively inhibits or moderately inhibits ABL1, ABL1-T315I, BTK, AURKA, JAK2 and JAK3; (iii) effectively inhibits or moderately inhibits JAK1; (iv) mildly inhibits or ineffectively inhibits JAK1; (v) effectively inhibits or moderately inhibits one or more of all of TYK2, a TRK family PK, ROS1, TXK, an EPH family PK, IRAK3, PLK4, TNK1, RET and JAK2-V617F; or (vi) a combination of (i) and (ii); (i) and (iii); (i) and (iv); (i) and (v); (i), (ii) and (iii); (i), (ii) and (iv); (i), (ii), (iii) and (v); or (i), (ii) and (iv) and (v).

L27.1 The use of any one of embodiments L1-L27, where the compound is of any one of embodiments A1-A109 and H1-H14.

L27.2. The use of any one of embodiments L1-L27.1, where the medical condition is treated with a compound of any one of embodiments A1-A109 and H1-H14.

L27.3. The use of any one of embodiments L1-L27.2, where the compound effectively inhibits ABL1, ABL1-T315I, AURKA, and JAK2.

L27.4. The use of any one of embodiments L1-L27.3, where the compound effectively inhibits or moderately inhibits BTK.

L27.5. The use of any one of embodiments L1-L27.4, where the compound effectively inhibits or moderately inhibits JAK3.

L27.6. The use of any one of embodiments L1-L27.5, where the compound effectively inhibits or moderately inhibits JAK1.

L27.7. The use of any one of embodiments L1-L27.5, where the compound mildly inhibits or ineffectively inhibits JAK1.

L27.8. The use of any one of embodiments L1-L27.7, where the compound effectively inhibits or moderately inhibits one or more or all of TYK2, A TRK family PK, ROS1, TXK, an EPH family PK, IRAK3, PLK4, TNK1, RET and JAK2-V617F.

L27.9. The use of any one of embodiments L1-L27.8, where the compound effectively inhibits or moderately inhibits one or more or all of TRKA, TRKB, TRKC, ROS1, EPHA1 and EPHB1.

L27.10. The use of any one of embodiments L1-L27.9, where the compound effectively inhibits or moderately inhibits a SRC family PK and/or a DDR family PK.

L27.11. The use of embodiment L27.10, where the DDR family PK is DDR2 PK and optionally a DDR2 variant PK.

L27.12. The use of embodiment L27.11, where the DDR2 variant PK contains a T654M and/or N456S amino acid substitution.

L27.13. The use of any one of embodiments L27.10-L27.12, where the SRC family PK is SRC(wt) or SRC-N1 (wt).

L27.14. The use of any one of embodiments L1-L27.13, where the compound effectively inhibits or moderately inhibits ABL2.

L27.15. The use of any one of embodiments L1-L27.14, where the compound effectively inhibits or moderately inhibits PTK2B.

L27.16. The use of any one of embodiments L1-L27.15, where the compound effectively inhibits or moderately inhibits ABL1, ABL1-T315I, ABL2, TYK2, JAK2, TRKC and PTK2B.

L27.17. The use of any one of embodiments L1-L27.16, where the compound effectively inhibits or moderately inhibits ABL1, ABL1-T315I, BTK, AURKA, JAK2, NTRK3, PTK2B, TYK2 and ABL2.

L27.18. The use of embodiment L27.17, where the NTRK3 is a ETV6-NTRK3 fusion.

L27.19. The use of any one of embodiments L1-L27.18, where the a compound of any one of embodiments B1-B16, F1-F15 and H1-H14.

L28. The use of any one of embodiments L6-L8, where the cancer is associated with a PLK family PK aberration and optionally of a PLK4 family PK aberration.

L29. The use of any one of embodiments L6-L8 and L28, where the cancer is a liver cancer, breast cancer or AML.

L30. The use of any one of embodiments L6-L8, where the cancer is associated with an IRAK family PK aberration and optionally an IRAK3 family PK aberration.

L30.1. The use of any one of embodiments L28-L30, where the compound effectively inhibits or moderately inhibits a PLK4 family PK or an IRAK3 family PK.

L31. The use of any one of embodiments L1-L11 and L28-L30.1, where the compound is of any one of embodiments G300-G318 and H1-H14.

L32. The use of any one of embodiments L30-L31, comprising treatment in combination with an immune checkpoint blockade (ICB) therapeutic.

L33. The use of any one of embodiments L1-L11, where the medical condition is associated with a JAK2 aberration.

L34. The use of embodiment L33, where the compound effectively inhibits JAK2, ineffectively inhibits or mildly inhibits JAK1, and optionally: moderately inhibits JAK3 and/or JAK2-V617F.

L34.1. The use of embodiment L34, where the compound effectively inhibits or moderately inhibits one or more or all of TXK, JAK2-V617F, TRKB, TRKC, ROS1, AURKA, IRAK3 and TNK1.

L34.2. The use of embodiment L34 or L34.1, where the compound is of any one of embodiments C1-C11 and H1-H14.

L34.3. The use of embodiment L33, where the compound effectively inhibits JAK2, ineffectively inhibits or mildly inhibits JAK1, and optionally: effectively inhibits JAK3 and/or JAK2-V617F.

L34.4. The use of embodiment L34.3, where the compound is of any one of embodiments D1-D12 and H1-H14.

K34.5. The use of embodiment L33, where the compound effectively inhibits JAK2, ineffectively inhibits or mildly inhibits JAK1, and optionally: moderately inhibits JAK3 and/or mildly inhibits JAK2-V617F.

K34.6. The use of embodiment K34.5, where the compound effectively inhibits or moderately inhibits one or more or all of TRKB, TRKC, AURKA, IRAK3 and PLK4.

L34.7. The use of embodiment L34.5 or L34.6, where the compound is of any one of embodiments E1-E11 and H1-H14.

L34.8. The use of embodiment L33, where the compound effectively inhibits JAK2, JAK3, JAK2-V617F and mildly inhibits JAK1.

K34.9. The use of embodiment K34.8, where the compound effectively inhibits or moderately inhibits one or more or all of ABL1, TRKA, TRKB, TRKC, ROS1, AURKA, IRAK3, TNK1.

L34.10. The use of embodiment L34.9, where the compound is of any one of embodiments G1-G10 and H1-H14.

L35. The use of embodiment L33, where the compound effectively inhibits JAK2, moderately inhibits JAK1, and optionally: moderately inhibits JAK3 and/or moderately inhibits or effectively inhibits JAK2-V617F.

L35.1. The use of embodiment L35, where the compound is of any one of embodiments G100-G110 and H1-H14.

L35.2. The use of embodiment L33, where the compound effectively inhibits or moderately inhibits TXK.

L35.3. The use of embodiment L35.2, where the compound effectively inhibits JAK2, for ineffectively inhibits or mildly inhibits JAK1, and optionally: for effectively inhibits or moderately inhibits JAK3 and/or JAK2-V617F.

L35.4. The use of embodiment L35.2 or L35.3, where the compound is of any one of embodiments G200-G209 and H1-H14.

L36. The use of any one of embodiments L6-L8 and L33-L35.4, where the medical condition is a cancer.

L37. The use of embodiment L36, where the cancer is a lung cancer, breast cancer, head cancer or neck cancer.

L38. The use of embodiment L36, where the cancer is a blood cancer.

L39. The use of embodiment L38, where the blood cancer is myelodysplastic syndrome (MDS), myelofibrosis, polycythemia vera or essential thrombocythemia.

L40. The use of embodiment L39, where the blood cancer is positive for a JAK2 variant comprising a V617F substitution.

L40.1. The use of any one of embodiments L38-L40, where the compound effectively inhibits JAK2, ineffectively inhibits or mildly inhibits JAK1, and optionally: effectively inhibits JAK3 and/or JAK2-V617F.

L40.2. The use of embodiment L40.1, where the compound is of any one of embodiments D1-D12 and H1-H14.

L40.3. The use of any one of embodiments L38-L40, where the compound effectively inhibits JAK2, moderately inhibits JAK1, and optionally: moderately inhibits JAK3 and/or moderately inhibits or effectively inhibits JAK2-V617F.

L40.4. The use of embodiment L40.3, where the compound is of any one of embodiments G100-G110 and H1-H14.

L42. The use of any one of embodiments L1-L41, where the medical condition is minimum residual disease (MRD).

L43. The use of any one of embodiments L1-L5 and L33-L34, where the medical condition is an inflammation condition, autoimmune condition and/or skin condition.

L44. The use of embodiment L43, where: the inflammation condition is a chronic inflammation condition, senescent cell chronic inflammation condition or senescence-associated secretory phenotype condition; the autoimmune condition is atopic dermatitis, non-segmental vitiligo or rheumatoid arthritis; or the skin condition is atopic dermatitis, non-segmental vitiligo, psoriasis, plaque psoriasis, ultraviolet (UV) damaged skin, severely UV damaged skin, aged skin or severely aged skin.

L45. The use of embodiment L43 or L44, where the medical condition is a cytokine-associated condition, and optionally an interleukin-6 (IL-6) associated medical condition.

L46. The use of embodiment L44 or L45, where the skin condition is atopic dermatitis or non-segmental vitiligo.

L46.1. The use of embodiment L46, where the compound effectively inhibits JAK2, ineffectively inhibits or mildly inhibits JAK1, and optionally: moderately inhibits JAK3 and/or JAK2-V617F.

L46.2. The use of embodiment L46, where the compound effectively inhibits or moderately inhibits one or more or all of TXK, JAK2-V617F, TRKB, TRKC, ROS1, AURKA, IRAK3 and TNK1.

L47. The use of embodiment L46.1 or L46.2, where the compound is of any one of embodiments C1-C11 and H1-H14.

L47.1. The use of embodiment L46 or L46.1, where the compound effectively inhibits JAK2, ineffectively inhibits or mildly inhibits JAK1, and optionally: effectively inhibits JAK3 and/or JAK2-V617F.

L47.2. The use of embodiment L47.1, where the compound is of any one of embodiments D1-D12 and H1-H14.

L48. The use of embodiment L44 or L45, where the autoimmune condition is rheumatoid arthritis.

L49. The use of embodiment L48, where the condition is moderate to severe rheumatoid arthritis and/or rheumatoid arthritis intolerant to a tumor necrosis factor (TNF) inhibitor.

L49.1. The use of embodiment L48 or L49, where the compound effectively inhibits JAK2, JAK3, JAK2-V617F and mildly inhibits JAK1.

K49.2. The use of embodiment K49.1, where the compound effectively inhibits or moderately inhibits one or more or all of ABL1, TRKA, TRKB, TRKC, ROS1, AURKA, IRAK3, TNK1.

L49.3. The use of embodiment L49.1 or L49.2, where the compound is of any one of embodiments G1-G10 and H1-H14.

L49.4. The use of embodiment L48 or L49, where the compound effectively inhibits JAK2, moderately inhibits JAK1, and optionally: moderately inhibits JAK3 and/or moderately inhibits or effectively inhibits JAK2-V617F.

L49.5. The use of embodiment L49.4, where the compound is of any one of embodiments G100-G110 and H1-H14.

L50. The use of embodiment L48 or L49, where the compound effectively inhibits or moderately inhibits TXK.

L50.1. The use of embodiment L50, where the compound effectively inhibits JAK2, for ineffectively inhibits or mildly inhibits JAK1, and optionally: for effectively inhibits or moderately inhibits JAK3 and/or JAK2-V617F.

L50.2. The use of embodiment L50 or L50.1, where the compound is of any one of embodiments G200-G209 and H1-H14.

L51. The use of embodiment L33 or L34, where the medical condition is associated with a TYK family PK aberration and optionally is associated with a TYK2 PK aberration.

L51.1. The use of any one of embodiments L33, L34 and L51, where the medical condition is a psoriasis, optionally a moderate to severe psoriasis, optionally a moderate to severe plaque psoriasis, and/or optionally is treated in a subject who is a candidate for systemic therapy or phototherapy.

K51.2. The use of embodiment L51 or L51.1, where the compound effectively inhibits or moderately inhibits TYK2.

K51.3. The use of embodiment K51.2, where the compound effectively inhibits or moderately inhibits JAK3.

K51.4. The use of embodiment K51.2 or K51.3, where the compound mildly inhibits or ineffectively inhibits JAK1 and/or JAK2.

K51.5. The use of any one of embodiments K51.2—K51.4, where the compound effectively inhibits or moderately inhibits one or more or all of ABL1-T315I, AURKA, PLK4 and IRAK3.

L52. The use of any one of embodiments L51-L51.5, where the compound is of any one of embodiments G400-G414 and H1-H14.

L53. The use of any one of embodiments L1-L52, where the composition comprises a compound that selectively inhibits a JAK2 PK.

L54. The use of embodiment L53, where the compound inhibits, and optionally effectively inhibits, JAK2(wt) PK, does not effectively inhibit JAK (wt) PK, and optionally effectively inhibits or moderately inhibits JAK3(wt) PK.

L55. The use of embodiment L53 or L54, where the medical condition is treated with a lower incidence of a serious adverse event, as compared to treatment of the medical condition with a compound that is not a selective inhibitor of a JAK2 PK.

L56. The use of embodiment L55, where the serious adverse event is one or more of: a malignancy, serious adverse cardiovascular event and/or blood clot, mortality and infection.

L57. The use of any one of embodiments L53-L56, where the medical condition is treated in a subject having a prior history of heart disease.

L57.1. The use of any one of embodiments L53-L57, where the compound effectively inhibits JAK2, ineffectively inhibits or mildly inhibits JAK1, and optionally: moderately inhibits JAK3 and/or JAK2-V617F.

L57.2. The use of any one of embodiments L53-L57, where the compound effectively inhibits or moderately inhibits one or more or all of TXK, JAK2-V617F, TRKB, TRKC, ROS1, AURKA, IRAK3 and TNK1.

L57.3. The use of embodiment L57.1 or L57.2, where the compound is of any one of embodiments C1-C11 and H1-H14.

L57.4. The use of any one of embodiments L53-L57, where the compound effectively inhibits JAK2, ineffectively inhibits or mildly inhibits JAK1, and optionally: effectively inhibits JAK3 and/or JAK2-V617F.

L57.5. The use of embodiment L57.4, where the compound is of any one of embodiments D1-D12 and H1-H14.

K57.6. The use of any one of embodiments L53-L57, where the compound effectively inhibits JAK2, ineffectively inhibits or mildly inhibits JAK1, and optionally: moderately inhibits JAK3 and/or mildly inhibits JAK2-V617F.

K57.7. The use of embodiment K57.6, where the compound effectively inhibits or moderately inhibits one or more or all of TRKB, TRKC, AURKA, IRAK3 and PLK4.

L57.8. The use of embodiment L57.6 or L57.7, where the compound is of any one of embodiments E1-E11 and H1-H14.

L57.9. The use of any one of embodiments L53-L57, where the compound effectively inhibits JAK2, JAK3, JAK2-V617F and mildly inhibits JAK1.

K57.10. The use of embodiment K57.9, where the compound effectively inhibits or moderately inhibits one or more or all of ABL1, TRKA, TRKB, TRKC, ROS1, AURKA, IRAK3, TNK1.

L57.11. The use of embodiment L57.10, where the compound is of any one of embodiments G1-G10 and H1-H14.

L57.12. The use of any one of embodiments L53-L57, where the compound effectively inhibits JAK2, moderately inhibits JAK1, and optionally: moderately inhibits JAK3 and/or moderately inhibits or effectively inhibits JAK2-V617F.

L58. The use of embodiment L57.12, where the compound is of any one of embodiments G100-G110 and H1-H14.

L50. The use of any one of embodiments L53-L57, where the compound effectively inhibits or moderately inhibits TXK.

L50.1. The use of embodiment L50, where the compound effectively inhibits JAK2, for ineffectively inhibits or mildly inhibits JAK1, and optionally: for effectively inhibits or moderately inhibits JAK3 and/or JAK2-V617F.

L58. The use of embodiment L50 or L50.1, where the compound is of any one of embodiments G200-G209 and H1-H14.

L59. The use of any one of embodiments L1-L52, where the compound inhibits, and optionally effectively inhibits, JAK2(wt), and optionally JAK1(wt) and optionally JAK3(wt). L60. The use of embodiment L59, where the composition comprises a compound of any one of embodiments B1-B16, F1-F15 and H1-H14.

PA1. A composition comprising a compound of Formula PA1 or Formula PA2:

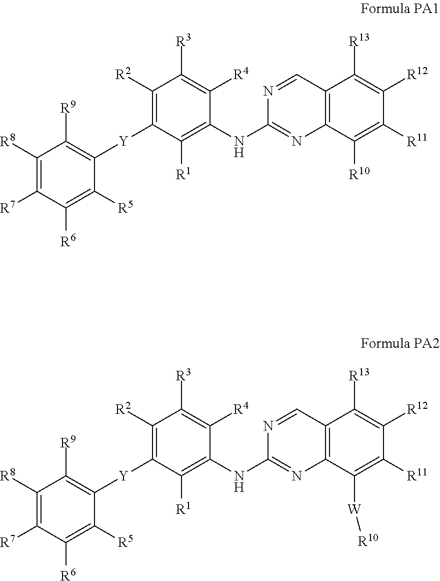

or a pharmaceutically acceptable salt, amide or ester thereof, wherein:
R¹, R², R³ and R⁴ each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, hydroxy, halo, cyano, amino or amido;
Y is —C(O)N(R^{b})—, —N(R^{b})C(O)— or —N(R^{b})—CH₂—;
R^{b} is H, optionally substituted alkyl or optionally substituted alkynyl;
R⁵, R⁶, R⁸ and R⁹ each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —B(OH)₂, —CO₂H, —C(O)OR^{u}, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl;
R⁷ is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —B(OH)₂, —C(O)OR^{u}, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl;
R^{u} is an optionally substituted alkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl; W is an optionally substituted alkylene, optionally substituted alkynyl, amino, amido, —O—, —
S—, —S(O)— or —SO₂—;
R¹⁰ is an optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, —B(OH)₂, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; and
R¹¹, R¹² and R¹³ each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, —B(OH)₂, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl.

PA2. The composition of embodiment PA1, wherein W is an optionally substituted alkylene or optionally substituted alkynyl.

PA3. The composition of embodiment PA1 or PA2, wherein R¹⁰ is an optionally substituted lower alkyl, optionally substituted lower alkenyl, optionally substituted lower alkynyl, optionally substituted lower alkoxy, optionally substituted lower heteroalkyl, optionally substituted lower haloalkyl, optionally substituted lower deuteroalkyl, optionally substituted lower alkylthio, optionally substituted lower alkylamino, optionally substituted lower hydroxyalkyl, optionally substituted lower mercaptoalkyl, optionally substituted lower aminoalkyl, —CO₂H, —B(OH)₂, hydroxy, halo, cyano, amino, amido, optionally substituted lower cycloalkyl, optionally substituted lower heterocycloalkyl, optionally substituted lower aryl, optionally substituted lower heteroaryl, optionally substituted lower arylaminoalkyl, optionally substituted lower aryloxyalkyl, optionally substituted lower arylthioalkyl, optionally substituted lower heteroarylaminoalkyl, optionally substituted lower heteroaryloxyalkyl, optionally substituted lower heteroarylthioalkyl, optionally substituted lower arylalkyl, and optionally substituted lower heteroarylalkyl.

PA4. The composition of embodiment PA1, wherein W is amino, amido, —O—, —S—, —S(O)— or —SO₂—.

PA5. The composition of any one of embodiments PA1-PA4, wherein R¹⁰ is an optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, and optionally substituted heteroarylalkyl.

PA6. The composition of embodiment PA5, wherein R¹⁰ is an optionally substituted lower alkyl, optionally substituted lower alkenyl, optionally substituted lower alkynyl, optionally substituted lower heteroalkyl, optionally substituted lower haloalkyl, optionally substituted lower deuteroalkyl, optionally substituted lower hydroxyalkyl, optionally substituted lower mercaptoalkyl, optionally substituted lower aminoalkyl, optionally substituted lower cycloalkyl, optionally substituted lower heterocycloalkyl, optionally substituted lower aryl, optionally substituted lower heteroaryl, optionally substituted lower arylaminoalkyl, optionally substituted lower aryloxyalkyl, optionally substituted lower arylthioalkyl, optionally substituted lower heteroarylaminoalkyl, optionally substituted lower heteroaryloxyalkyl, optionally substituted lower heteroarylthioalkyl, optionally substituted lower arylalkyl, and optionally substituted lower heteroarylalkyl.

PA7. The composition of any one of embodiments PA1-PA6, wherein R¹, R², R³ and R⁴ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl or halo.

PA8. The composition of any one of embodiments PA1-PA7, wherein R¹, R², R³ and R⁴ each independently is H, methyl, methoxy, Cl, F, CF₃ or CD₃.

PA9. The composition of any one of embodiments PA1-PA8, wherein R^{b} is H, methyl or ethyl.

PA10. The composition of any one of embodiments PA1-PA9, wherein R^{b} is H or methyl.

PA11. The composition of any one of embodiments PA1-PA10, wherein R⁵, R⁶, R⁷, R⁸ and R⁹ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{c}C(O)N(R^{d})—, —C(O)N(R^{c}R^{d}), —NR^{e}R^{f}, —B(OH)₂, —C(O)OR^{u}, hydroxy, halo, cyano, optionally substituted C5-C7 cycloalkyl, or optionally substituted heterocycloalkyl containing 5 to 7 ring member atoms; R^{c}, R^{d}, R^{e} and R^{f} each independently is H or optionally substituted C1-C6 alkyl; and R^{u} is an optionally substituted C1-C6 alkyl.

PA12. The composition of any one of embodiments PA1-PA12, wherein R⁵, R⁶, R⁷, R⁸ and R⁹ each independently is H, N-methyl piperazine, R^{c}C(O)N(R^{d})—, —C(O)N(R^{c}R^{d}), —C(O)OR^{u}, —NR^{e}R^{f} or cyano; R^{c}, R^{d}, R^{e} and R^{f} each independently is H or optionally substituted C1-C6 alkyl; and R^{u} is an optionally substituted C1-C6 alkyl.

PA13. The composition of any one of embodiments PA1-PA12, wherein R¹⁰ is an optionally substituted C5-C7 aryl, optionally substituted heteroaryl containing 5 to 7 ring atoms, optionally substituted C5-C7 cycloalkyl, optionally substituted heterocycloalkyl containing 5 to 7 ring atoms, R^{g}C(O)N(R^{h})—, —C(O)N(R^{g}R^{h}), —NRⁱR^{j}, cyano or halo; and R^{g}, R^{h}, Rⁱ and R^{j} each independently is H or optionally substituted C1-C6 alkyl.

PA14. The composition of embodiment PA13, wherein R^{g}, R^{h}, Rⁱ and R^{j} each independently is H or methyl.

PA15. The composition of any one of embodiments PA1-PA12, wherein R¹⁰ is an optionally substituted C5-C7 aryl, optionally substituted heteroaryl containing 5 to 7 ring atoms, optionally substituted C5-C7 cycloalkyl, or optionally substituted heterocycloalkyl containing 5 to 7 ring atoms.

PA16. The composition of any one of embodiments PA1-PA15, wherein R¹⁰ is halo; or is a C6 aryl or heteroaryl containing 6 ring member atoms, each optionally substituted with one or more of optionally substituted alkyl, Cl, F, CF₃, CD₃, methoxy, isopropyloxy and dimethylamino; or is a heteroaryl containing 5 ring member atoms optionally substituted with one or more of optionally substituted alkyl, Cl, F, CF₃, CD₃, methoxy, isopropyloxy and dimethylamino.

PA17. The composition of any one of embodiments PA1-PA16, wherein R¹¹, R¹² and R¹³ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, —B(OH)₂, hydroxy, halo, cyano, optionally substituted C5-C7 cycloalkyl, or optionally substituted heterocycloalkyl containing 5 to 7 ring member atoms; and R^{k}, R^{m}, R^{p} and R^{q} each independently is H or optionally substituted C1-C6 alkyl.

PA18. The composition of embodiment PA17, wherein R^{k}, R^{m}, R^{p} and R^{q} each independently is H or methyl.

PA19. The composition of any one of embodiments PA1-PA18, wherein R¹¹, R¹² and R¹³ each independently is H, optionally substituted alkyl, Cl, F, CF₃, CD₃, methoxy, isopropyloxy or dimethylamino.

PA20. The composition of any one of embodiments PA1-PA16, wherein W is —CH₂—, —C≡C—, —NH(R^{t})—, —O—, —S—, —S(O)— or —SO₂—; and R^{t} is H or optionally substituted C1-C6 alkyl.

PA21. The composition of any one of embodiments PA1-PA20, wherein R^{t} is H or methyl.

PA22. The composition of any one of embodiments PA1-PA21, wherein Y is —C(O)N(H)— or —N(H)C(O)—.

PA23. The composition of any one of embodiments PA1-PA22, wherein: R¹⁰ is according to Formula PB:

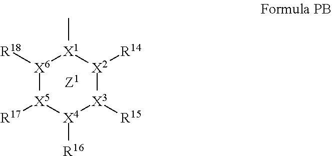

Z¹ is aryl or heteroaryl;
X¹, X², X³, X⁴, X⁵ and X⁶ each independently is C or N; and
R¹⁴, R¹⁵, R¹⁶, R¹⁷, and R¹⁸ each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, —B(OH)₂, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl; or two adjacent R¹⁴, R¹⁵, R¹⁶, R¹⁷, and R¹⁸ are linked in an optionally substituted aryl or optionally substituted heteroaryl.

PA24. The composition of embodiment PA23, wherein zero, one or two of X¹, X², X³, X⁴, X⁵ and X⁶ is N and X¹, X², X³, X⁴, X⁵ and X⁶ that are not N are C.

PA25. The composition of embodiment PA23 or PA24, wherein Z¹ is aryl and each of X¹, X², X³, X⁴, X⁵ and X⁶ is C.

PA26. The composition of any one of embodiments PA23-PA25, wherein R¹⁴, R¹⁵, R¹⁶, R¹⁷, and R¹⁸ each independently is H, optionally substituted C1-C6 alkyl, optionally substituted C1-C6 alkoxy, C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, —B(OH)₂, hydroxy, halo, cyano, optionally substituted C5-C6 cycloalkyl, optionally substituted heterocycloalkyl containing 5 or 6 ring member atoms; wherein R^{k}, R^{m}, R^{p} and R^{q} each independently is H or optionally substituted C1-C6 alkyl.

PA27. The composition of embodiment PA26, wherein R^{k}, R^{m}, R^{p} and R^{q} each independently is H or methyl.

PA28. The composition of any one of embodiments PA23-PA27, wherein R¹⁴, R¹⁵, R¹⁶, R¹⁷, and R¹⁸ each independently is H, optionally substituted C1-C6 alkyl, Cl, F, CF₃, CD₃, methoxy, isopropyloxy or dimethylamino.

PA29. The composition of embodiment PA23 or PA24, wherein R¹⁴, R¹⁵, R¹⁶, R¹⁷, and R¹⁸ each independently is H, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl.

PA30. The composition of embodiment PA29, wherein R¹⁴, R¹⁵, R¹⁶, R¹⁷, and R¹⁸ each independently is H, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, or optionally substituted heterocycloalkyl containing 5 or 6 ring member atoms.

PA31. The composition of embodiment PA30, wherein R¹⁴, R¹⁵, R¹⁶, R¹⁷, and R¹⁸ each independently is H or optionally substituted C1-C6 alkyl.

PA32. The composition of any one of embodiments PA1-PA22, wherein: R¹⁰ is according to Formula PC:

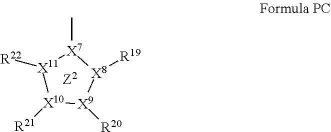

Z² is aryl or heteroaryl;
X⁷, X⁸, X⁹, X¹⁰ and X¹¹ each independently is C, N, O or S; and
R¹⁹, R²⁰, R²¹ and R²² each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, —B(OH)₂, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl; or two adjacent R¹, R¹⁵, R¹⁶, R¹⁷, and R¹⁸ are linked in an optionally substituted aryl or optionally substituted heteroaryl.

PA33. The composition of embodiment PA32, wherein zero, one or two of X⁷, X⁸, X⁹, X¹⁰ and X¹¹ is N and X⁷, X⁸, X⁹, X¹⁰ and X¹¹ that are not N are C.

PA34. The composition of embodiment PA32 or PA33, wherein Z² is heteroaryl; X⁷, X¹⁰ and X¹¹ each is C, and one of X⁸ and X⁹ is N and the other is C.

PA35. The composition of embodiment PA29, wherein X⁸ and X⁹ each is N.

PA36. The composition of any one of embodiments PA32-PA35, wherein R¹⁹, R²⁰, R²¹ and R²² each independently is H, optionally substituted C1-C6 alkyl, optionally substituted C1-C6 alkoxy, optionally substituted C1-C6 heteroalkyl, optionally substituted C1-C6 deuteroalkyl, optionally substituted C1-C6 haloalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, —B(OH)₂, hydroxy, halo, cyano, optionally substituted C5-C6 cycloalkyl, or optionally substituted heterocycloalkyl containing 5 or 6 ring member atoms; and R^{k}, R^{m}, R^{p} and R^{q} each independently is H or optionally substituted C1-C6 alkyl.

PA37. The composition of embodiment PA36, wherein R^{k}, R^{m}, R^{p} and R^{q} each independently is H or methyl.

PA38. The composition of any one of embodiments PA32-PA37, wherein R¹⁹, R²⁰, R²¹ and R²² each independently is H, optionally substituted C1-C6 alkyl, Cl, F, CF₃, CD₃, methoxy, isopropyloxy or dimethylamino.

PA39. The composition of any one of embodiments PA32-PA35, wherein R¹⁹, R²⁰, R²¹ and R²² each independently is H, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl.

PA40. The composition of embodiment PA39, wherein R¹⁹, R²⁰, R²¹ and R²² each independently is H, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, or optionally substituted heterocycloalkyl containing 5 or 6 ring member atoms.

PA41. The composition of embodiment PA40, wherein R¹⁹, R²⁰, R²¹ and R²² each independently is H or optionally substituted C1-C6 alkyl.

PA42. The composition of any one of embodiments PA1-PA22, wherein: R¹⁰ is according to Formula PD:

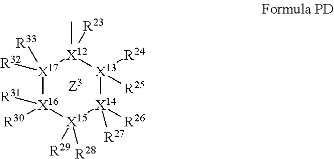

Z³ is cycloalkyl or heterocycloalkyl;
X¹² is C or N;
X¹³, X¹⁴, X¹⁵, X¹⁶ and X¹⁷ each independently is C, N, O or S; and
R²³, R²⁴, R²⁵, R²⁶, R²⁷, R²⁸, R²⁹, R³⁰, R³¹, R³² and R³³ each optionally is present, and when present, each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; or two adjacent R¹⁴, R¹⁵, R¹⁶, R¹⁷, and R¹⁸ are linked in an optionally substituted aryl or optionally substituted heteroaryl.

PA43. The composition of embodiment PA42, wherein zero, one or two of X¹², X¹³, X¹⁴, X¹⁵, X¹⁶ and X¹⁷ is N and X¹², X¹³, X¹⁴, X¹⁵, X¹⁶ and X¹⁷ that are not N are C.

PA44. The composition of embodiment PA42 or PA43, wherein Z³ is cycloalkyl and each of X¹², X¹³, X¹⁴, X¹⁵, X¹⁶ and X¹⁷ is C.

PA45. The composition of any one of embodiments PA42-PA44, wherein R²³, R²⁴, R²⁵, R²⁶, R²⁷, R²⁸, R²⁹, R³⁰, R³¹, R³² and R³³ each optionally is present, and when present, each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, hydroxy, halo, cyano, optionally substituted C5-C6 cycloalkyl, optionally substituted heterocycloalkyl containing 5 or 6 ring member atoms; wherein R^{k}, R^{m}, R^{p} and R^{p} each independently is H or optionally substituted C1-C6 alkyl.

PA46. The composition of embodiment PA45, wherein R^{k}, R^{m}, R^{p} and R^{q} each independently is H or methyl.

PA47. The composition of any one of embodiments PA42-PA46, wherein R²³, R²⁴, R²⁵, R²⁶, R²⁷, R²⁸, R²⁹, R³⁰, R³¹, R³² and R³³ each independently is H, optionally substituted C1-C6 alkyl, Cl, F, CF₃, CD₃, methoxy, isopropyloxy or dimethylamino.

PA48. The composition of embodiment PA42 or PA43, wherein R²³, R²⁴, R²⁵, R²⁶, R²⁷, R²⁸, R²⁹, R³⁰, R³¹, R³² and R³³ each optionally is present, and when present, each independently is H, optionally substituted alkyl, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl.

PA49. The composition of embodiment PA48, wherein R²³, R²⁴, R²⁵, R²⁶, R²⁷, R²⁸, R²⁹, R³⁰, R³¹, R³² and R³³ each optionally is present, and when present, each independently is H, optionally substituted C1-C6 alkyl, optionally substituted C1-C6 heteroalkyl, optionally substituted C1-C6 haloalkyl, C optionally substituted 1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, or optionally substituted heterocycloalkyl containing 5 or 6 ring member atoms.

PA50. The composition of embodiment PA49, wherein R²³, R²⁴, R²⁵, R²⁶, R²⁷, R²⁸, R²⁹, R³⁰, R³¹, R³² and R³³ each optionally is present, and when present, each independently is H or optionally substituted C1-C6 alkyl.

PA51. The composition of any one of embodiments PA1-PA22, wherein: R¹⁰ is according to Formula PE:

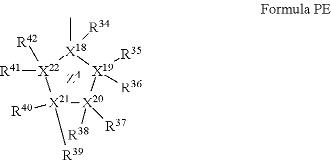

Z⁴ is cycloalkyl or heterocycloalkyl;
X¹⁸ is C or N;
X¹⁹, X²⁰, X²¹ and X²² each independently is C, N, O or S; R³⁴, R³⁵, R³⁶, R³⁷, R³⁸, R³⁹, R⁴⁰, R⁴¹ and R⁴² each optionally is present or absent, and when present, each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, optionally substituted heteroarylalkyl; or two adjacent R¹⁴, R¹⁵, R¹⁶, R¹⁷, and R¹⁸ are linked in an optionally substituted aryl or optionally substituted heteroaryl.

PA52. The composition of embodiment PA51, wherein zero, one or two of X¹⁸, X¹⁹, X²⁰, X²¹, and X²² is N and X¹⁸, X¹⁹, X²⁰, X²¹, and X²² that are not N are C.

PA53. The composition of embodiment PA51 or PA52, wherein Z⁴ is cycloalkyl and X¹⁸, X¹⁹, X²⁰, X²¹, and X²² each is C.

PA54. The composition of any one of embodiments PA51-PA53, wherein R³⁴, R³⁵, R³⁶, R³⁷, R³⁸, R³⁹, R⁴⁰, R⁴¹ and R⁴² each optionally is present, and when present, each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, hydroxy, halo, cyano, optionally substituted C5-C6 cycloalkyl, optionally substituted heterocycloalkyl containing 5 or 6 ring member atoms; wherein R^{k}, R^{m}, R^{p} and R^{q} each independently is H or optionally substituted C1-C6 alkyl.

PA55. The composition of embodiment PA54, wherein R^{k}, R^{m}, R^{p} and R^{q} each independently is H or methyl.

PA56. The composition of any one of embodiments PA51-PA55, wherein R³⁴, R³⁵, R³⁶, R³⁷, R³⁸, R³⁹, R⁴⁰, R⁴¹ and R⁴² each independently is H, optionally substituted C1-C4 alkyl, Cl, F, CF₃, CD₃, methoxy, isopropyloxy or dimethylamino.

PA57. The composition of embodiment PA40 or PA41, wherein R³⁴, R³⁵, R³⁶, R³⁷, R³⁸, R³⁹, R⁴⁰, R⁴¹ and R⁴² each optionally is present, and when present, each independently is H, optionally substituted alkyl, optionally substituted alkenyl, optionally substituted alkynyl, heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl, or optionally substituted heteroarylalkyl.

PA58. The composition of embodiment PA57, wherein R³⁴, R³⁵, R³⁶, R³⁷, R³⁸, R³⁹, R⁴⁰, R⁴¹ and R⁴² each optionally is present, and when present, each independently is H, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, or optionally substituted heterocycloalkyl containing 5 or 6 ring member atoms.

PA59. The composition of embodiment PA58, wherein R³⁴, R³⁵, R³⁶, R³⁷, R³⁸, R³⁹, R⁴⁰, R⁴¹ and R⁴² each optionally is present, and when present, each independently is H or optionally substituted C1-C6 alkyl.

PA60. The composition of any one of embodiments PA1-PA22, wherein R¹⁰ is halo.

PA61. The composition of embodiment PA60, wherein R¹⁰ is bromine.

PA62. The composition of any one of embodiments PA1-PA61, wherein W is —CH₂— or —C≡C—.

PA63. The composition of any one of embodiments PA1-PA62, wherein R⁵, R⁶, R⁷, R⁸ and R⁹ each independently is H, R^{c}C(O)N(R^{d})—, —C(O)N(R^{c}R^{d}) or —C(O)OR^{u}; R^{c} and R^{d} each independently is H or optionally substituted C1-C6 alkyl; and R^{u} is an optionally substituted C1-C6 alkyl.

PA64. The composition of any one of embodiments PA1-PA63, wherein R^{c}, R^{d}, R^{e} and R^{f} each independently is H or optionally substituted C1-C4 alkyl and R^{u} is an optionally substituted C1-C4 alkyl.

PA65. The composition of any one of embodiments PA1-PA63, wherein R⁵, R⁶, R⁷, R⁸ and R⁹ each independently is H, R^{c}C(O)N(R^{d})— or —C(O)N(R^{c}R^{d}); and R^{c} and R^{d} each independently is H or optionally substituted C1-C6 alkyl.

PA66. The composition of any one of embodiments PA1-PA65, wherein one of R⁵, R⁶, R⁷, R⁸ and R⁹ is R^{c}C(O)N(R^{d})— or —C(O)N(R^{c}R^{d}); and R^{c} and R^{d} each independently is H or optionally substituted C1-C6 alkyl.

PA67. The composition of embodiment PA66, wherein R⁷ is R^{c}C(O)N(R^{d})— or —C(O)N(R^{c}R^{d}); and R^{c} and R^{d} each independently is H or optionally substituted C1-C6 alkyl.

PA68. The composition of embodiment PA67, wherein R⁵, R⁶, R⁸ and R⁹ each is H.

PA69. The composition of any one of embodiments PA65-PA68, wherein R^{c} and R^{d} each independently is H or optionally substituted C1-C4 alkyl.

PA70. The composition of any one of embodiments PA65-PA69, wherein R^{c} and R^{d} each independently is H or methyl.

PA71. The composition of any one of embodiments PA1-PA70, with the proviso that R⁷ is not —CO₂H.

PA72. The composition of any one of embodiments PA1-PA71, with the proviso that R² and R³, or optionally R³ and R⁴, do not join to form an imidazolyl group.

PA73. The composition of any one of embodiments PA1-PA72, with the proviso that R¹⁰ is not methyl.

PA74. The composition of any one of embodiments PA1-PA72, with the proviso that R¹⁰ is not alkyl.

PA75. The composition of any one of embodiments PA1-PA72, with the proviso that R¹⁰ is not methoxy.

PA76. The composition of any one of embodiments PA1-PA72, with the proviso that R¹⁰ is not alkoxy.

PA77. The composition of any one of embodiments PA1-PA72, with the proviso that R¹⁰ is not an unsubstituted phenyl.

PA78. The composition of any one of embodiments PA1-PA72, wherein the compound is of Formula PA2 with the proviso that R¹⁰ is not an phenyl substituted with —C≡CH₃ and W is not amino.

PA79. The composition of any one of embodiments PA1-PA78, with the proviso that R² and R⁹ do not form a bond.

PA80. The composition of any one of embodiments PA1-PA79, with the proviso that (i) the amino group joined to the carbon ring atom in the quinazolinyl group positioned between the two nitrogen ring atoms of the quinazolinyl group by a covalent bond, and (ii) R¹ do not participate in a five-membered ring.

PB1. A composition comprising a compound of Formula PF:

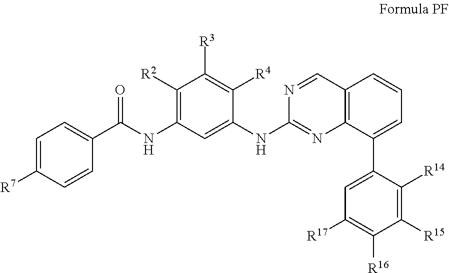

or a pharmaceutically acceptable salt, amide or ester thereof, wherein:
R², R³ and R⁴ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl or halo;
R⁷ is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{c}C(O)N(R^{d})—, —C(O)N(R^{c}R^{d}), —NR^{e}R^{f}, —C(O)OR^{u}, —B(OH)₂, hydroxy, halo, cyano, optionally substituted C5-C6 cycloalkyl, or optionally substituted heterocycloalkyl containing 5 or 6 ring member atoms;
R¹⁴, R¹⁵, R¹⁶ and R¹⁷ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, —B(OH)₂, hydroxy, halo, cyano, optionally substituted C5-C6 cycloalkyl, or optionally substituted heterocycloalkyl containing 5 or 6 ring member atoms;
R^{c}, R^{d}, R^{e}, R^{f}, R^{k}, R^{m}, R^{p} and R^{q} each independently is H or optionally substituted C1-C6 alkyl; and
R^{u} is an optionally substituted C1-C6 alkyl.

PB2. The composition of embodiment PB1 or PB2, wherein R², R³ and R⁴ each independently is H, methyl, methoxy, Cl, F, CF₃ or CDs.

PB3. The composition of embodiment PB1 or PB2, wherein R⁷ is N-methyl piperazine, R^{c}C(O)N(R^{d})—, —C(O)N(R^{c}R^{d}), —NR^{e}R^{f} or cyano; and R^{c}, R^{d}, R^{e} and R^{f} each independently is H or optionally substituted C1-C6 alkyl.

PB4. The composition of any one of embodiments PB1-PB3, wherein R⁷ is R^{c}C(O)N(R^{d})—, —C(O)N(R^{c}R^{d}) or —C(O)OR^{u}; R^{c} and R^{d} each independently is H or optionally substituted C1-C6 alkyl; and R^{u} is an optionally substituted C1-C6 alkyl.

PB5. The composition of any one of embodiments PB1-PB3, wherein R⁷ is R^{c}C(O)N(R^{d})— or —C(O)N(R^{c}R^{d}); and R^{c} and R^{d} each independently is H or optionally substituted C1-C6 alkyl.

PB6. The composition of any one of embodiments PB1-PB5, wherein R^{C}, R^{d}, R^{e}, R^{f}, R^{k}, R^{m}, R^{p} and R^{q} each independently is H or methyl.

PB7. The composition of any one of embodiments PB1-PB6, wherein R¹⁴, R¹⁵ and R¹¹ each independently is H, optionally substituted C1-C4 alkyl, Cl, F, CF₃, CDs, methoxy, isopropyloxy or dimethylamino.

PB8. The composition of any one of embodiments PB1-PB7, wherein R^{u} is an optionally substituted C1-C4 alkyl.

PB9. The composition of any one of embodiments PB1-PB8, wherein one of R², R³ and R⁴ is methyl or methoxy and the other two of R², R³ and R⁴ are H, or R², R³ and R⁴ each is H.

PB10. The composition of any one of embodiments PB1-PB9, wherein R¹⁵ is H.

PB11. The composition of any one of embodiments PB1-PB10, wherein R¹⁷ is H.

PB12. The composition of any one of embodiments PB1-PB11, with the proviso that R⁷ is not —CO₂H.

PC1. A composition comprising a compound of Formula PG:

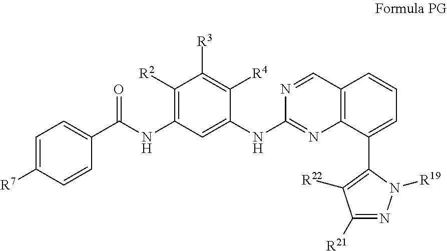

or a pharmaceutically acceptable salt, amide or ester thereof, wherein:
R², R³ and R⁴ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl or halo;
R⁷ is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{c}C(O)N(R^{d})—, —C(O)N(R^{c}R^{d}), —NR^{e}R^{f}, —C(O)OR^{u}, —B(OH)₂, hydroxy, halo, cyano, optionally substituted C5-C6 cycloalkyl, or optionally substituted heterocycloalkyl containing 5 or 6 ring member atoms;
R¹⁹, R²¹ and R²² each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, —B(OH)₂, hydroxy, halo, cyano, optionally substituted C5-C6 cycloalkyl, or optionally substituted heterocycloalkyl containing 5 or 6 ring member atoms;
R^{c}, R^{d}, R^{e}, R^{f}, R^{k}, R^{m}, R^{p} and R^{p} each independently is H or optionally substituted C1-C6 alkyl; and
R^{u} is an optionally substituted C1-C6 alkyl.

PC2. The composition of embodiment PC1 or PC2, wherein R², R³ and R⁴ each independently is H, methyl, methoxy, Cl, F, CF₃ or CDs.

PC3. The composition of embodiment PC1 or PC2, wherein R⁷ is N-methyl piperazine, R^{c}C(O)N(R^{d})—, —C(O)N(R^{c}R^{d}), —NR^{e}R^{f} or cyano; and R^{c}, R^{d}, R^{e} and R^{f} each independently is H or optionally substituted C1-C6 alkyl.

PC4. The composition of any one of embodiments PC1-PC3, wherein R⁷ is R^{c}C(O)N(R^{d})—, —C(O)N(R^{c}R^{d}) or —C(O)OR^{u}; R^{c} and R^{d} each independently is H or optionally substituted C1-C6 alkyl; and R^{u} is an optionally substituted C1-C6 alkyl.

PC5. The composition of any one of embodiments PC1-PC3, wherein R⁷ is R^{c}C(O)N(R^{d})— or —C(O)N(R^{c}R^{d}); and R^{c} and R^{d} each independently is H or optionally substituted C1-C6 alkyl.

PC6. The composition of any one of embodiments PC1-PC5, wherein R^{c}, R^{d}, R^{e}, R^{f}, R^{k}, R^{m}, R^{p} and R^{q} each independently is H or methyl.

PC7. The composition of any one of embodiments PC1-PC6, wherein R¹⁹, R²¹ and R²² each independently is H, optionally substituted C1-C4 alkyl, Cl, F, CF₃, CDs, methoxy, isopropyloxy or dimethylamino.

PC8. The composition of any one of embodiments PC1-PC7, wherein R^{u} is an optionally substituted C1-C4 alkyl.

PC9. The composition of any one of embodiments PC1-PC8, wherein one of R², R³ and R⁴ is methyl or methoxy and the other two of R², R³ and R⁴ are H, or R², R³ and R⁴ each is H.

PC10. The composition of any one of embodiments PC1-PC9, with the proviso that R⁷ is not —CO₂H.

PD1. A composition comprising N1-(5-((8-(4-fluoro-2-isopropoxyphenyl)quinazolin-2-yl)amino)-2-methylphenyl)-N4-methylterephthalamide.

PD2. A composition comprising N1-(5-((8-(4-fluoro-2-isopropoxyphenyl)quinazolin-2-yl)amino)-2-methylphenyl)-N4,N4-dimethylterephthalamide.

PD3. A composition comprising N-(5-((8-(4-fluoro-2-isopropoxyphenyl)quinazolin-2-yl)amino)-2-methylphenyl)terephthalamide.

PD4. A composition comprising ethyl 4-((5-((8-bromoquinazolin-2-yl)amino)-2-methylphenyl)carbamoyl)benzoate.

PD5. A composition comprising ethyl 4-((3-((8-bromoquinazolin-2-yl)amino)-5-methylphenyl)carbamoyl)benzoate.

PD6. A composition comprising ethyl 4-((5-((8-bromoquinazolin-2-yl)amino)-2-methoxyphenyl)carbamoyl)benzoate.

PD7. A composition comprising ethyl 4-((3-((8-bromoquinazolin-2-yl)amino)phenyl)carbamoyl)benzoate.

PD8. A composition comprising N1-[5-[(8-bromoquinazolin-2-yl)amino]-2-methyl-phenyl]-N4-methyl-terephthalamide.

PD9. A composition comprising N4-[5-[(8-bromoquinazolin-2-yl)amino]-2-methyl-phenyl]-N1,N1-dimethyl-terephthalamide.

PE1. A composition comprising a compound of Formula PH:

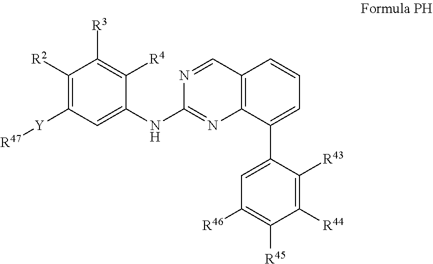

or a pharmaceutically acceptable salt thereof, wherein:
R², R³ and R⁴ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl or halo;
Y is —C(O)N(R^{b})—, —N(R^{b})C(O)— or —N(R^{b})—CH₂—;
R⁴³, R⁴⁴, R⁴⁵ and R⁴⁶ each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, —B(OH)₂, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl; or two adjacent R⁴³, R⁴⁴, R⁴⁵ and R⁴⁶ are linked in an optionally substituted aryl or optionally substituted heteroaryl;
R^{b}, R^{k}, R^{m}, R^{p} and R^{q} each independently is H or optionally substituted C1-C6 alkyl;
R⁴⁷ is H, optionally substituted alkyl containing at least 2 carbon atoms, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted alkoxyalkyl, optionally substituted amidoalkyl, optionally substituted cycloalkyl, optionally substituted heterocycloalkyl, optionally substituted cycloalkylalkyl, or optionally substituted heterocycloalkylalkyl.

PE2. The composition of embodiment PE1, wherein Y is —N(R^{b})—CH₂—.

PE3. The composition of embodiment PE2, wherein R^{b} is H.

PE4. The composition of any one of embodiments PE1-PE3, with the proviso that Y and R⁴⁷ do not together form

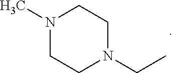

PE5. The composition of any one of embodiments PE1-PE3, with the proviso that Y and R⁴⁷ do not together form

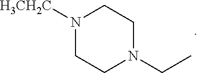

PE6. The composition of any one of embodiments PE1-PE3, with the proviso that Y and R⁴⁷ do not together form

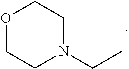

PE7. The composition of any one of embodiments PE1-PE3, with the proviso that Y and R⁴⁷ do not together form

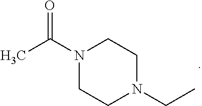

PE8. A composition comprising a compound of Formula J:

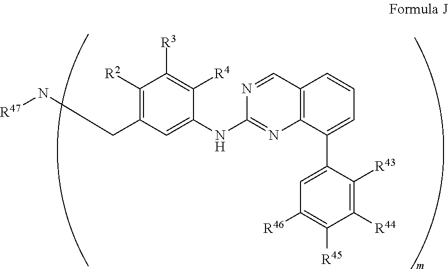

or a pharmaceutically acceptable salt thereof, wherein:
R², R³ and R⁴ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl or halo;
R⁴³, R⁴⁴, R⁴⁵ and R⁴⁶ each independently is H, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —CO₂H, —B(OH)₂, hydroxy, halo, cyano, amino, amido, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl; or two adjacent R⁴³, R⁴⁴, R⁴⁵ and R⁴⁶ are linked in an optionally substituted aryl or optionally substituted heteroaryl;
R^{k}, R^{m}, R^{p} and R^{q} each independently is H or optionally substituted C1-C6 alkyl;
m is an integer of 1 or 2; and,
R⁴⁷ is H, optionally substituted alkyl containing at least 2 carbon atoms, optionally substituted alkenyl, optionally substituted alkynyl, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted alkoxyalkyl, optionally substituted amidoalkyl, optionally substituted cycloalkyl, optionally substituted heterocycloalkyl, optionally substituted cycloalkylalkyl, or optionally substituted heterocycloalkylalkyl.

PE9. The composition of any one of embodiments PE1-PE8, wherein R^{b}, R^{k}, R^{m}, R^{p} and R^{a} each independently is H or methyl.

PE10. The composition of any one of embodiments PE1-PE9, wherein R², R³ and R⁴ each independently is H, methyl, methoxy, Cl, F, CF₃ or CD₃.

PE11. The composition of any one of embodiments PE1-PE10, wherein one of R², R³ and R⁴ is methyl or methoxy and the other two of R², R³ and R⁴ are H, or R², R³ and R⁴ each is H.

PE12. The composition of any one of embodiments PE1-PE11, wherein R⁴³, R⁴⁴, R⁴⁵ and R⁴⁶ each independently is H, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{k}C(O)N(R^{m})—, —C(O)N(R^{k}R^{m}), —NR^{p}R^{q}, —CO₂H, —B(OH)₂, hydroxy, halo, cyano, optionally substituted C5-C6 cycloalkyl, or optionally substituted heterocycloalkyl containing 5 or 6 ring member atoms.

PE13. The composition of any one of embodiments PE1-PE12, wherein R⁴³, R⁴⁴, R⁴⁵ and R⁴⁶ each independently is H, optionally substituted C1-C4 alkyl, Cl, F, CF₃, CD₃, methoxy, isopropyloxy or dimethylamino.

PE14. The composition of any one of embodiments PE1-PE12, wherein R⁴⁴ and R⁴⁶ each is an optionally substituted C1-C6 alkoxy.

PE15. The composition of embodiment PE14, wherein R⁴⁴ and R⁴⁶ each is methoxy.

PE16. The composition of any one of embodiments PE1-PE15, wherein R⁴³ is H, or R⁴⁵ is H, or R⁴³ and R⁴⁵ each is H.

PE17. The composition of any one of embodiments PE1-PE16, wherein R⁴⁷ is H, optionally substituted C1-C6 alkyl, optionally substituted C1-C6 alkoxyalkyl, optionally substituted C1-C6 heteroalkyl, optionally substituted C1-C6 haloalkyl, optionally substituted C1-C6 deuteroalkyl or optionally substituted C1-C6 hydroxyalkyl.

PE18. The composition of any one of embodiments PE1-PE17, with the proviso that R⁴⁷ is not a phenyl or substituted phenyl.

PE19. The composition of any one of embodiments PE1-PE18, with the proviso that R⁴⁵ is not methoxy.

PE20. The composition of any one of embodiments PE1-PE19, with the proviso that R⁴⁵ is not

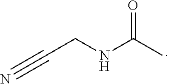

PE21. The composition of any one of embodiments PE1-PE20, with the proviso that R⁴⁵ is not

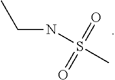

PE22. The composition of any one of embodiments PE1-PE21, with the proviso that R⁴⁵ is not

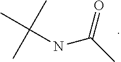

PE23. The composition of any one of embodiments PE1-PE22, with the proviso that R⁴⁴ or R⁴⁶ is not

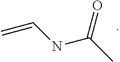

PE24. The composition of any one of embodiments PE1-PE23, with the proviso that R² is not

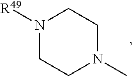

wherein R⁴⁹ is methyl, —CH₂CH₂OH, —CH₂CH₂OCH₃, —CH₂CH₂(OH)CH₃, —CH₂CH₂(OH)(CH₃)CH₃, or —CH₂CH₂F.

PE25. The composition of any one of embodiments PE1-PE24, with the proviso that R⁴⁷ is not methyl.

PE26. The composition of any one of embodiments PE1-PE25, with the proviso that R⁴⁷ does not contain a polyethylene glycol.

PE27. The composition of any one of embodiments PE1-PE26, with the proviso that the compound is not of the following formula:

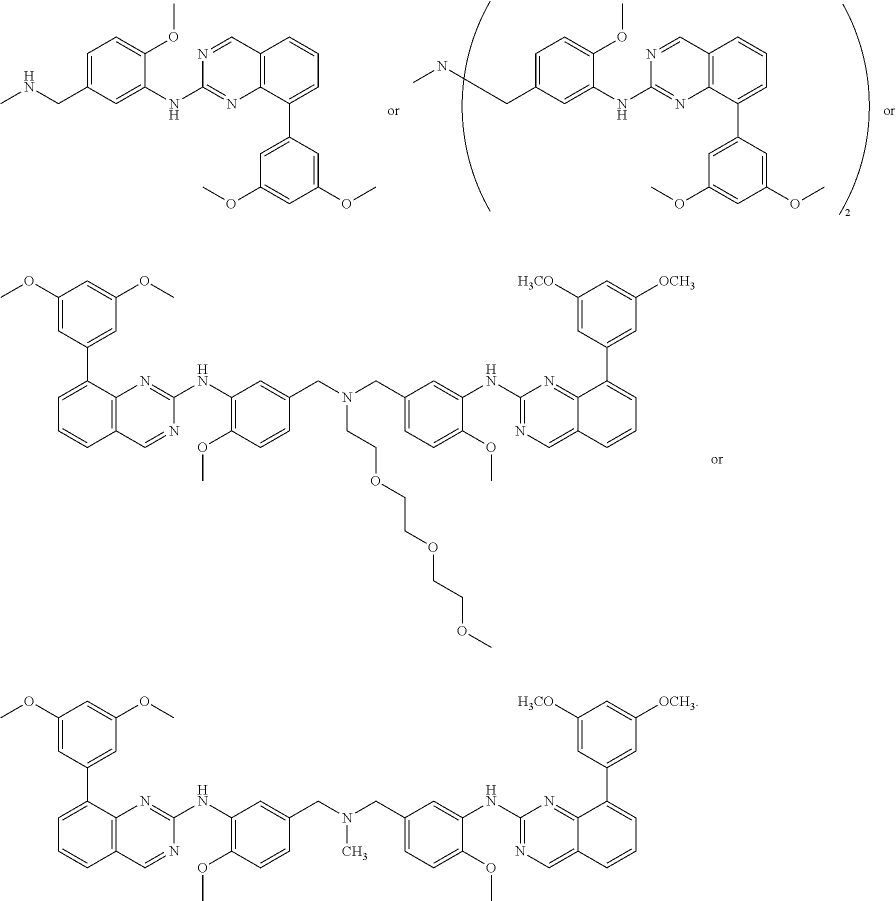

PE28. The composition of any one of embodiments PE8-PE27, wherein m is the integer 2.

PF1. The composition of any one of embodiments PA1-PA80, PB1-PB12, PC1-PC10, PD1-PD9 and PE1-PE28, which is a pharmaceutical composition.

PF2. The composition of any one of embodiments PA1-PA80, PB1-PB12, PC1-PC10, PD1-PD9 and PE1-PE28, wherein the compound binds to and inhibits an activity of an ABL1 polypeptide.

PF3. The composition of embodiment PF2, wherein the compound binds to and inhibits an activity of two or more ABL1 variant polypeptides.

PF4. The composition of embodiment PF4, wherein the compound is an effective inhibitor of two or more ABL1 variant polypeptides.

PF5. The composition of any one of embodiments PA1-PA80, PB1-PB12, PC1-PC10, PD1-PD9, PE1-PE28 and PF1-PF4, for inhibition of an activity of an ABL1 polypeptide or ABL1 variant polypeptide.

PF6. The composition of embodiment PF5, for inhibition of catalytic activity of an ABL1 polypeptide or ABL1 variant polypeptide.

PF7. The composition of any one of embodiments PA1-PA80, PB1-PB12, PC1-PC10, PD1-PD9, PE1-PE28 and PF1-PF4, for treatment of a condition.

PF8. The composition of embodiment PF7, wherein the condition is an ABL1-related condition.

PF9. The composition of embodiment PF7 or PF8, wherein the condition is a cancer.

PF10. The composition of embodiment PF9, wherein the cancer is a leukemia.

PF11. The composition of embodiment PF10, wherein the leukemia is chronic myeloid leukemia.

PF12. The composition of embodiment PF10, wherein the leukemia is acute lymphoblastic leukemia.

### EXAMPLES

The examples below illustrate certain implementations and do not limit the technology.

### Example 1: Preparation of Cmpd10

Described in this Example is a process for preparing Cmpd10 shown in Table A.

### Preparation of tert-butyl N-[4-methyl-3-[[4-(methylcarbamoyl)benzoyl]amino]phenyl]carbamate

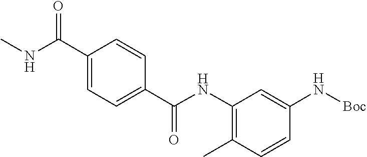 [SMILES: CNC(=O)c1ccc(C(=O)Nc2cc(NC(=O)OC(C)(C)C)ccc2C)cc1]

A 20 mL scintillation vial was charged with tert-butyl N-(3-amino-4-methyl-phenyl)carbamate (500 mg, 2.25 mmol) followed by 4-(methylcarbamoyl)benzoic acid (725 mg, 4.05 mmol). DMF (7 mL) was added and the mixture stirred until dissolution was complete. TEA (560 μL, 4.05 mmol) was added followed by EEDQ (1.00 g, 4.05 mmol). The initially homogenous yellow solution became a suspension of a finely divided off-white solid within 2 hours (hr). The mixture was stirred at room temperature (rt) overnight.

LCMS showed a major product with the expected mass. The mixture was diluted with excess iPrOAc, yielding a white suspension. The suspension was washed with saturated aqueous sodium bicarbonate (3×), then water, then 1N HCl (3×), then water. The mixture remained as a white organic suspension with the aqueous layers separating cleanly, with no emulsion. The organic suspension was dried over MgSO₄ and filtered, yielding a clear filtrate. The filtrate was evaporated to a tan colored semi-solid (143 mg).

The MgSO₄ in the funnel, presumably mixed with the white suspended material, was dissolved in 1N HCl, leaving the insoluble white suspended material. The mixture was evaporated free of iPrOAc and filtered. The derived white solid, which was the desired product, was dried under high vacuum overnight (332 mg).

### Preparation of N1-(5-amino-2-methyl-phenyl)-N4-methyl-terephthalamide

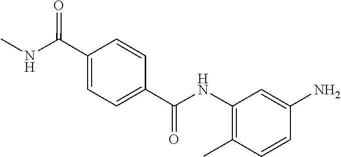 [SMILES: CNC(=O)c1ccc(C(=O)Nc2cc(N)ccc2C)cc1]

A 20 mL screw-top scintillation vial was charged with tert-butyl N-[4-methyl-3-[[4-(methylcarbamoyl)benzoyl]amino]phenyl]carbamate (332 mg, 0.866 mmol) followed by DCM (9 mL). The stirred suspension was treated with TFA (2 mL). The suspended solid dissolved yielding a clear, pale brown solution. The solution was stirred at rt for 1 hour.

LCMS showed the reaction was complete, with two peaks of identical mass and isotopic distribution.

The solution was treated with toluene (9 mL) and evaporated to a dark oil. The oil was rinsed into a separatory funnel containing excess EtOAc and saturated aqueous sodium bicarbonate, causing the formation of a voluminous off-white precipitate which obscured the phase separation.

The two-phase mixture was filtered with rinsing using excess EtOAc. The derived off-white solid was pull-dried for 2 hours until it became granular and could be removed from the filter funnel with a spatula. The solid was digested in excess IPA at reflux, causing most of it to dissolve and leaving behind some insoluble material. The IPA solution was allowed to cool to rt and filtered. The filtrate was evaporated to a white solid (189 mg).

LCMS showed two widely separated peaks with each peak having an identical mass and conforming to the theoretical isotopic distribution (M+H) corresponding to the desired compound. The product was used without further purification.

### Preparation of N1-[5-[(8-bromoquinazolin-2-yl)amino]-2-methyl-phenyl]-N4-methyl-terephthalamide (Cmpd17)

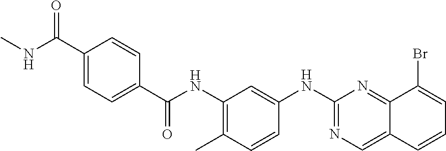 [SMILES: CNC(=O)c1ccc(C(=O)Nc2cc(Nc3ncc4cccc(Br)c4n3)ccc2C)cc1]

A 0.5 mL-2.0 mL tapered microwave vial with triangular stir vane was charged with N1-(5-amino-2-methyl-phenyl)-N4-methyl-terephthalamide (44 mg, 0.155 mmol). EtOH (1 mL) was added and the mixture stirred to produce a suspension. 8-bromo-2-chloro-quinazoline (189 mg, 0.776 mmol) was added and the mixture sealed, then heated in an aluminum block (125° C.) for 6 hours.

The reaction was a mixture of a yellow supernatant and a voluminous light yellow solid. The mixture was recovered from the reaction vial by rinsing with excess EtOH into a 40 mL scintillation vial. Addition of DCM did not lead to significant dissolution. A representative sample was prepared by removing some of the suspension from the scintillation vial and carrying out two serial dilutions with DCM and then into an LCMS vial with addition of DMSO to effect dissolution. LCMS showed a single major peak with the correct mass and isotopic distribution.

The reaction mixture was rendered free of volatiles by evaporation. IPA (10 mL) was added and the mixture brought to a boil, producing a suspended light yellow solid in a yellow supernatant. The mixture was stirred at rt for 1 hour. The precipitated solid was collected by gravity filtration and rinsed with minimal IPA in the filter funnel. The funnel with the solid was wrapped with a tissue on top and placed under lyophilizer vacuum for 1 hr.

Most of the solid could be removed from the funnel (48 mg). Some of it stuck to the frit and was washed through with DCM (13 mg).

The two materials were shown to be identical by LCMS and were combined (61 mg). The product was used without further purification.

### Preparation of N1-[5-([[8-(4-fluoro-2-isopropoxy-phenyl)quinazolin-2-yl]amino]-2-methyl-phenyl]-N4-methyl-terephthalamide (Cmpd10)

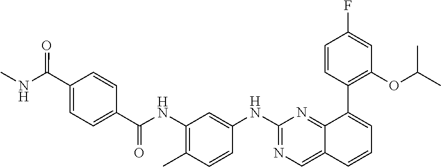 [SMILES: CNC(=O)c1ccc(C(=O)Nc2cc(Nc3ncc4cccc(-c5ccc(F)cc5OC(C)C)c4n3)ccc2C)cc1]

A 0.5 mL-2.0 mL tapered microwave vial with triangular stir vane was charged with a suspension of N1-[5-[(8-bromoquinazolin-2-yl)amino]-2-methyl-phenyl]-N4-methyl-terephthalamide (61 mg, 0.124 mmol) in dioxane (2 mL). (4-fluoro-2-isopropoxy-phenyl)boronic acid (37 mg, 0.187 mmol) was added followed by aqueous K₃PO₄ (2.0M, 190 μL, 0.373 mmol). Tetrakis(triphenylphosphine)palladium (29 mg, 0.025 mmol) was added and the vial was sealed, then placed with stirring in a 105° C. aluminum block for 5 hours.

LCMS of the dark solution showed the presence of the desired product. The 2-phase mixture was diluted with excess iPrOAc and dried over MgSO₄. Filtration and evaporation afforded a solid/gummy residue mixture. The material was transferred to a 20 mL scintillation vial with hot EtOAc and pumped down to the same mixture as before (108 mg).

The mixture was digested at reflux in IPA (7 mL). Most of the solid, but not all, dissolved. The mixture was stirred at rt for 1 hr causing a voluminous tan solid to precipitate. The mixture was filtered by gravity with IPA rinsing. The filter funnel with the light tan solid was allowed to stand under lyophilizer vacuum for 1 hour. The solid was transferred to a 20 mL scintillation vial (44 mg).

LCMS showed the desired product was present along with a minor amount of another compound which apparently formed in this reaction. The solid was digested at reflux in acetone and then stirred at rt for 2 hr. The vial was clamped at a 45 degree angle in the hood and allowed to stand at rt for over 48 hr.

LCMS of the supernatant showed almost all the impurity was gone. The supernatant was drawn off and the solid was digested in hot acetone 2× more. The combined supernatants were filtered. The solution was evaporated, and the derived light yellow solid sampled for LCMS. The desired product was present with acceptable purity (19 mg).

### Example 2: Preparation of Cmpd11

Described in this Example is a process for preparing Cmpd11 shown in Table A.

### Preparation of tert-butyl N-[3-[[4-(dimethylcarbamoyl)benzoyl]amino]-4-methylphenyl]carbamate

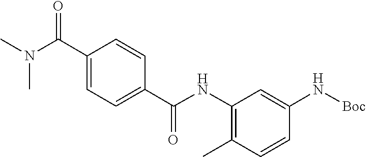 [SMILES: Cc1ccc(NC(=O)OC(C)(C)C)cc1NC(=O)c1ccc(C(=O)N(C)C)cc1]

A 40 mL scintillation vial was charged with tert-butyl N-(3-amino-4-methyl-phenyl)carbamate (640 mg, 2.20 mmol) followed by 4-(dimethylcarbamoyl)benzoic acid (1 g, 5.18 mmol). DMF (9 mL) was added and the mixture stirred until dissolution was complete. TEA (720 μL, 5.18 mmol) was added followed by EEDQ (1.28 g, 5.18 mmol). The initially homogenous yellow solution became a suspension of a finely divided off-white solid within 2 hours. The mixture was stirred at rt overnight.

LCMS showed a major product with the expected mass. The mixture was diluted with excess iPrOAc, yielding a suspension of a white solid. The suspension was washed with saturated aqueous bicarbonate (3×), then water, then 1N HCl (3×), then water. The mixture remained as a white organic suspension with the aqueous layers separating cleanly, and with no emulsion. The organic suspension was dried over MgSO₄ and filtered, yielding a clear filtrate. The filtrate was evaporated to a tan colored semi-solid.

The MgSO₄ in the funnel, presumably mixed with the white suspended material, was dissolved in 1N HCl, leaving the white suspended material. The mixture was filtered. The derived white solid was dried under high vacuum overnight (259 mg).

### Preparation of N4-(5-amino-2-methyl-phenyl)-N1,N1-dimethyl-terephthalamide

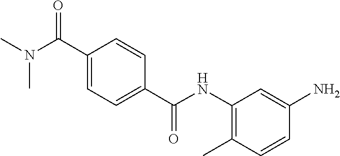 [SMILES: Cc1ccc(N)cc1NC(=O)c1ccc(C(=O)N(C)C)cc1]

A 40 mL screw top vial was charged with (dimethylcarbamoyl)benzoyl]amino]-4-phenyl]carbamate (0.259 g, 0.6 mmol). DCM (2.2 mL) was added and the mixture stirred. TFA (0.44 mL) was added and the mixture was stirred until dissolution was complete. The vial was sealed and stirred at rt for 1 hr. LCMS showed the reaction had a substantial amount of starting material so TFA (0.44 mL) was added and stirred at rt for 1 hr. LCMS indicated the reaction was complete. Toluene (10 mL) was added to the reaction and evaporated. Ethyl acetate (~50 mL) was added and then the solution was washed with saturated aqueous sodium bicarbonate (1×). The organic phase was dried over MgSO₄ and filtered. The filtrate was evaporated to a yellow powder (194 mg). The product was used without further purification.

### Preparation of N4-[5-[(8-bromoquinazolin-2-yl)amino]-2-methyl-phenyl]-N1,N1-dimethyl-terephthalamide (Cmpd18)

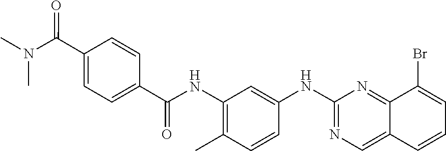 [SMILES: Cc1ccc(Nc2ncc3cccc(Br)c3n2)cc1NC(=O)c1ccc(C(=O)N(C)C)cc1]

A 20 mL scintillation vial was charged with a solution of N4-(5-amino-2-methyl-phenyl)-N1,N1-dimethylterephthalamide (140 mg, 0.47 mmol) in EtOH (3 mL). Solid 8-bromo-2-chloro-quinazoline (571 mg, 2.35 mmol) was added. 7 mL EtOH was added to the solution which was sealed in a vial and heated to 125° C. in an aluminum block for 5 hours.

The dark brown, transparent solution was sampled for LCMS. LCMS showed that all of the aniline was consumed and the desired product was present. Upon cooling a green suspension was formed. It was rinsed with excess EtOH into a round bottom flask and evaporated. Excess DCM was used to transfer the residue into a 20 mL scintillation vial. The DCM was evaporated, leaving a brown-orange oil. IPA (2-3 mL) was added and the mixture was heated to dissolve the material. Upon cooling and stirring at rt for 2 hr, the resultant solid/liquid mixture was filtered. The filtrate was evaporated to a residue for normal phase separation using a 0%-5% mobile phase gradient (0%-5% MeOH in DCM, column volumes).

The solid remaining in the filter funnel was dissolved in excess DCM and filtered into a separate round-bottomed (rb) flask.

The solids left in the microwavable tube were layered with excess DCM and stirred at rt for an hour yielding an orange supernatant. This operation was repeated three more times with the supernatants being collectively recovered. This last mixture was filtered under vacuum, leaving behind a yellow solid in the funnel.

There were three isolated materials. The DCM soluble material from the filter funnel, the first IPA filtrate and the DCM filtrate from the insoluble solids. The DCM solutions were combined to afford the product (56 mg).

### Preparation of N1-(5-((8-(4-fluoro-2-isopropoxyphenyl)quinazolin-2-yl)amino)-2-methylphenyl)-N4,N4-dimethylterephthalamide (Cmpd11)

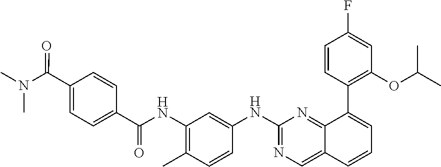 [SMILES: Cc1ccc(Nc2ncc3cccc(-c4ccc(F)cc4OC(C)C)c3n2)cc1NC(=O)c1ccc(C(=O)N(C)C)cc1]

A 0.5 mL-2.0 mL tapered microwave vial with triangular stir vane was charged with (4-fluoro-2-isopropoxyphenyl)boronic acid (72 mg, 0.362 mmol). A solution of ethyl 4-[[5-[(8-bromoquinazolin-2-yl)amino]-2-methylphenyl]carbamoyl]benzoate (122 mg, 0.241 mmol) in dioxane (0.7 mL) was added followed by aqueous K₃PO₄ (0.658 mL, 2M, 1.316 mmol). Tetrakis(triphenylphosphine)palladium (64.2 mg, 0.055 mmol) was added and the vial sealed, then heated in an aluminum block (105° C.) for 5 hours.

LCMS indicated the reaction was complete with the formation of the desired product. The reaction was diluted with excess iPrOAc. MgSO₄ was added and the mixture filtered and evaporated to a brown oil (158 mg). The oil was partially purified by absorption onto silica gel (650 mg) followed by elution with 2:1 Hex/EtOAc (10 mL). The derived residue was chromatographed by pTLC (500 micrometer plates) using 2:1 Hex/EtOAc. The major UV-active band was isolated affording the desired product (42 mg).

### Example 3: Preparation of Cmpd12

Described in this Example is a process for preparing Cmpd12 shown in Table A.

### Preparation of ethyl 4-[[5-[[8-(4-fluoro-2-isopropoxy-phenyl)quinazolin-2-yl]amino]-2-methyl-phenyl]carbamoyl]benzoate

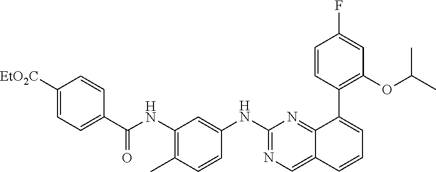 [SMILES: CCOC(=O)c1ccc(C(=O)Nc2cc(Nc3ncc4cccc(-c5ccc(F)cc5OC(C)C)c4n3)ccc2C)cc1]

A 0.5 mL-2.0 mL tapered microwave vial with triangular stir vane was charged with (4-fluoro-2-isopropoxy-phenyl)boronic acid (15 mg, 0.074 mmol). A solution of ethyl 4-[[5-[(8-bromoquinazolin-2-yl)amino]-2-methyl-phenyl]carbamoyl]benzoate (25 mg, 0.049 mmol; Cmpd13 described in Example 13) in dioxane (0.5 mL) was added followed by aqueous K₃PO₄ (0.074 mL, 2M, 0.15 mmol). Tetrakis(triphenylphosphine)palladium (13 mg, 0.011 mmol) was added and the vial sealed, then heated in an aluminum block (105° C.) for 5 hours. LCMS indicated the reaction was complete with the formation of the desired product. The reaction was diluted with excess iPrOAc. MgSO₄ was added and the mixture filtered and evaporated to a brown oil (50 mg). The oil was chromatographed on two 500 micrometer pTLC plates using 2:1 H/EA. The major UV active spot/band was isolated (yellow solid, 7 mg).

### Preparation of N-(5-((8-(4-fluoro-2-isopropoxyphenyl)quinazolin-2-yl)amino)-2-methylphenyl)terephthalamide (Cmpd12)

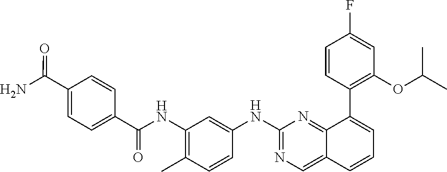 [SMILES: Cc1ccc(Nc2ncc3cccc(-c4ccc(F)cc4OC(C)C)c3n2)cc1NC(=O)c1ccc(C(N)=O)cc1]

At room temperature, a THF solution of a borane-ammonia complex is treated with sodium hydride (60% dispersion in mineral oil). With gas evolution, the initial mixture slowly becomes a slightly turbid solution. A THF solution of ethyl 4-[[5-[[8-(4-fluoro-2-isopropoxy-phenyl)quinazolin-2-yl]amino]-2-methyl-phenyl]carbamoyl]benzoate is added and the solution stirred for 10 minutes. Water is added followed by excess ethyl acetate. The organic phase is recovered, dried over MgSO₄, filtered and evaporated to afford the desired Cmpd12. Additional information pertaining to the process is in Nature Communications 2021, 12, pp 5964-5973.

### Example 4: Preparation of Compounds of Formula A1-2 in which R¹⁰ is R^{W} and R^{W} is Aryl or Heteroaryl

This Example describes a process for preparing a compound of Formula A12 having an optionally substituted aryl or optionally substituted heteroaryl R¹⁰ group from a compound of Formula A1 having a R¹⁰ bromo group (e.g., Cmpd13, Cmpd14, Cmpd15, Cmpd16, Cmpd17, Cmpd18; see Examples 13-16). The R¹⁰ group can be of Formula B or Formula C. The process includes a Suzuki coupling procedure.

A solution of the aryl bromide (1.0 eq; e.g., Cmpd13, Cmpd14, Cmpd15, Cmpd16, Cmpd17, Cmpd18) in 1,4-dioxane (0.20 M in aryl bromide) is placed in a sealed vessel with a magnetic stirring bar. The appropriate boronic acid/boronic ester (1.5 eq) is added to the vessel as a solid. A solution of K₃PO₄ (3 eq, 2M in water) is added followed by tetrakis(triphenylphosphine) palladium (0.2 eq). The vessel is sealed, stirred and heated in a aluminum block at about 105° C. for about 5 hours. The reaction mixture is recovered by dilution and rinsing with excess isopropyl acetate. The solution is dried over anhydrous MgSO₄, filtered and evaporated. The derived residue is purified by silica gel chromatography to afford the desired coupling product.

### Example 5: Preparation of Compounds of Formula A1-8

This Example describes a process for preparing a compound of Formula A1-8, which is a compound of Formula A1-2 in which R¹⁰ is —W—R^{W}, R^{W} is an optionally substituted aryl or optionally substituted heteroaryl, and W is —C≡C—, from a compound of Formula A1-2 having a R¹⁰ bromo group (e.g., Cmpd13, Cmpd14, Cmpd15, Cmpd16, Cmpd17, Cmpd18). A representative compound that can be prepared has the following structure:

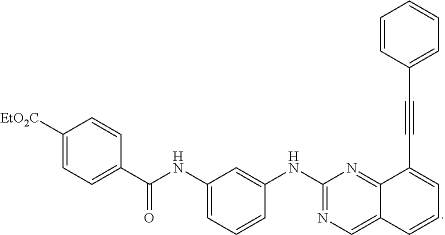 [SMILES: CCOC(=O)c1ccc(C(=O)Nc2cccc(Nc3ncc4cccc(C#Cc5ccccc5)c4n3)c2)cc1]

While a R^{W} phenyl group is shown above, the R^{W} group can be another aryl or heteroaryl group, which may be optionally substituted (e.g., an R^{W} group of Formula B2, B3, C2 or C3). The process includes a Sonogashira coupling procedure.

To a flask charged with the bromide substrate (e.g., Cmpd13, Cmpd14, Cmpd15, Cmpd16, Cmpd17, Cmpd18) is added Pd(PPh₃)₄ (0.10 equiv) and CuI (0.2 equiv) in a glovebox. The flask is removed from the glovebox and phenyl acetylene (1.3 equiv) is added, followed by NEt₃ (1.5 equiv) and DMF. The reaction mixture is sparged with N₂ for about 10 minutes (min), then stirred at about 60° C. for about 16 h. The reaction is allowed to cool to room temperature and quenched with saturated aqueous NH₄Cl, then diluted with H₂O and EtOAc. The layers are separated, and the aqueous layer is extracted with EtOAc. The combined organic layers are washed with brine and dried over Na₂SO₄. Evaporation under reduced pressure affords the crude product, which is purified by flash chromatography to provide the product.

### Example 6: Preparation of Compounds of Formula A1-2 in which R¹⁰ is —W—R^{W} and W is an Amino Group

This Example describes a process for preparing a compound of Formula A1-2 in which R¹⁰ is —W—R^{W}, R^{W} is an optionally substituted aryl or optionally substituted heteroaryl group, and W is an amino group (e.g., —N(H)—), from a compound of Formula A1-2 having a R¹⁰ bromo group (e.g., Cmpd13, Cmpd14, Cmpd15, Cmpd16, Cmpd17, Cmpd18). A representative compound that can be prepared, in which R^{W} is phenyl, has the following structure:

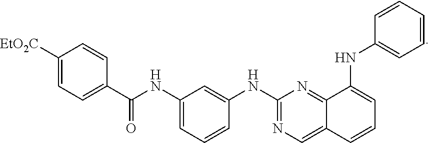 [SMILES: CCOC(=O)c1ccc(C(=O)Nc2cccc(Nc3ncc4cccc(Nc5ccccc5)c4n3)c2)cc1]

While a R^{W} phenyl group is shown above, the R^{W} group can be another aryl or heteroaryl group, which may be optionally substituted (e.g., a R^{W} group of Formula B2, B3, C2 or C3). The process includes a Buchwald amination procedure, and can make use of an aliphatic (saturated) amine.

A vessel equipped with a stir bar is charged with the bromide substrate (1.0 eq; e.g., Cmpd13, Cmpd14, Cmpd15, Cmpd16, Cmpd17, Cmpd18), amine (1.2 eq), cesium carbonate (1.5 eq), BippyPhos (0.10 eq), Pd(dba)₂ (0.10 eq) and DMF. The reaction vessel is sealed, heated to about 140° C., and stirred for about 16 h. The reactions are allowed to cool to room temperature, diluted with EtOAc and filtered through celite. The filtrate is evaporated to a residue and then purified by flash chromatography, affording the product.

### Example 7: Process for Preparing Compounds of Formula A1-2 in which R¹⁰ is —W—R^{W} and W is Sulfanyl

This Example describes a process for preparing a compound of Formula A1-2 in which R¹⁰ is —W—R^{W}, R^{W} is an optionally substituted aryl, optionally substituted heteroaryl, optionally substituted cycloalkyl, or optionally substituted heterocycloalkyl, and W is sulfanyl (i.e., —S—), from a substituted quinazoline. A representative compound that can be prepared, in which R^{W} is phenyl, has the following structure:

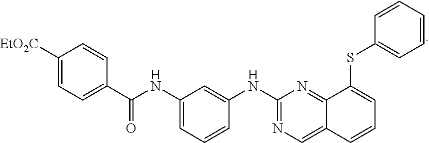 [SMILES: CCOC(=O)c1ccc(C(=O)Nc2cccc(Nc3ncc4cccc(Sc5ccccc5)c4n3)c2)cc1]

The process includes the following procedure.

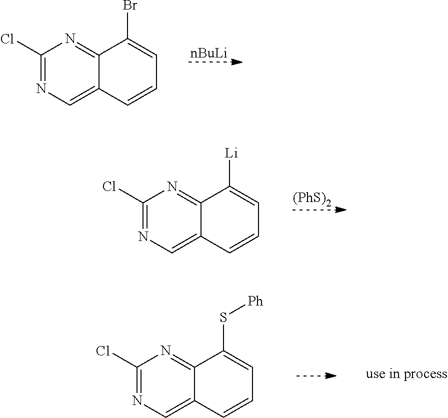

While a R^{W} phenyl group is shown above, the R^{W} group can be another aryl or heteroaryl group, or a cycloalkyl or heterocycloalkyl, which may be optionally substituted (e.g., an R^{W} group of Formula B2, B3, C2, C3, D2, D3, E2 or E3).

The chloro-bromo quinazoline is dissolved in THF and cooled to −78° C. in a dry ice/IPA bath. A solution of n-butyllithium in hexane is added and stirred at low temperature for 30 min. A solution of diphenyl disulfide in THF is added. After stirring for 30 min, the solution is allowed to warm slowly to 0° C. The reaction is quenched with a solution of acetic acid in THF. The crude reaction solution is partitioned between iPrOAc and saturated aqueous sodium bicarbonate. The organic phase is dried over MgSO₄, filtered and evaporated to a residue which is purified by silica gel chromatography.

The derived 8-phenylthio-2-chloroquinazoline is reacted with ethyl 4-((5-amino-2-methylphenyl)carbamoyl)benzoate, which is optionally substituted, to prepare compounds of Formula A1-2 in which W is —S—.

A representative general process for the reaction is as follows. A 40 mL screw top vial is charged with a solution of the chloroquinazoline and the ethyl 4-((5-amino-2-methylphenyl)carbamoyl)benzoate in ethanol. The vial is sealed and heated in an aluminum block (125° C.) for 5 hours. The mixture is cooled and diluted with excess ethanol, then DCM, leaving behind some dark, insoluble material. The extracts are filtered and evaporated to a dark solid. The solid is dissolved in IPA at reflux, then allowed to cool to rt and stir overnight. The precipitated solid product is recovered by filtration.

### Example 8: Process for Preparing Compounds of Formula A1-2 in which R¹⁰ is —W—R^{W} and W is Sulfanyl

This Example describes a process, as an alternative to the process described in Example 7, for preparing a compound of Formula A1-2, in which R¹⁰ is —W—R^{W}, R^{W} is an optionally substituted aryl, optionally substituted heteroaryl, optionally substituted cycloalkyl, or optionally substituted heterocycloalkyl, and W is sulfanyl (i.e., —S—). A representative compound that can be prepared, in which R^{W} is phenyl, has the following structure:

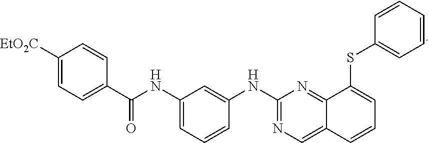 [SMILES: CCOC(=O)c1ccc(C(=O)Nc2cccc(Nc3ncc4cccc(Sc5ccccc5)c4n3)c2)cc1]

The process includes conversion of a compound of Formula A1-2 having a R¹⁰ bromo group (e.g., Cmpd13, Cmpd14, Cmpd15, Cmpd16, Cmpd17, Cmpd18). While a R^{W} phenyl group is shown above, the R^{W} group can be another aryl or heteroaryl group, or cycloalkyl or heterocycloalkyl, which may be optionally substituted (e.g., an R^{W} group of Formula B2, B3, C2, C3, D2, D3, E2 or E3).

A Schlenk tube is charged successively with K2CO₃ (74 mg, 0.5 mmol) and degassed xylene (2 mL). After purging with N₂ using 3 evacuate-fill cycles, the slurry is cooled to 0° C. and thiophenol (1 mmol) is added dropwise. The resulting mixture is then allowed to warm to room temperature and stirred for about 1 hr. A second Schlenk tube is charged successively with a compound of Formula A1 in which R¹⁰ is bromo (e.g., Cmpd13, Cmpd14, Cmpd15, Cmpd16, Cmpd17, Cmpd18) (0.8 mmol), Pd₂(dba)₃ (0.08 mmol), Xantphos (0.09 mmol) and degassed xylene (10 mL).

After purging with N2 using 3 evacuate-fill cycles, the mixture is stirred at room temperature for 20 min and transferred via a cannula to the previously formed potassium thiolate. The dark solution is then purged with N2 and heated to reflux for 24 h. After cooling to room temperature, the mixture is diluted with EtOAc (20 mL), washed with water (3×20 mL), dried over MgSO₄ and concentrated under reduced pressure. The crude product is then purified by silica gel chromatography to afford the anticipated thioether. Additional details are provided in Tetrahedron 2005, 61, pp 5253-5259, for example.

### Example 9: Process for Preparing Compounds of Formula A1-2 in which R¹⁰ is —W—R^{W}, W is Oxy and R^{W} is an Optionally Substituted Aryl or Heteroaryl Group

This Example describes a process for preparing a compound of Formula A1-2 in which R¹⁰ is —W—R^{W}, R^{W} is an optionally substituted aryl or optionally substituted heteroaryl, and W is oxy (i.e., —O—), from a substituted quinazoline. A representative product in which R^{W} is phenyl may be prepared by the following general process.

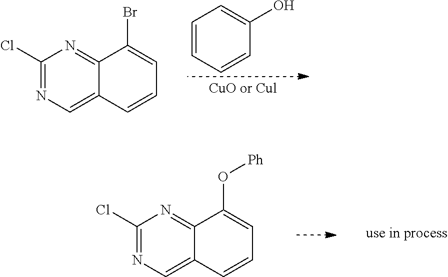

While a R^{W} phenyl group is shown above, the R^{W} group can be another aryl or heteroaryl group, which may be optionally substituted (e.g., an R^{W} group of Formula B2, C3, C2 or C3).

A vial is loaded with K3P04 (1.8 mmol), phenol (1.8 mmol), and the corresponding auxiliary ligand (0.18 mmol). Then, in an inert atmosphere glovebox, copper iodide (0.18 mmol) in DMSO and the quinazoline bromide (0.90 mmol) are added. The vial is sealed, and the reaction mixture is kept under an inert atmosphere and placed in a preheated aluminum block (115° C.). After the reaction mixture is stirred for about 24 hr, it is quenched by the addition of EtOAc (5 mL). The workup includes filtration of the reaction mixture through silica gel and purification of the derived crude product by flash chromatography. Additional details are provided in J Organic Chem 2016, 81, pp 7315-7325.

The derived 8-phenoxy-2-chloroquinazoline is reacted with ethyl 4-((5-amino-2-methylphenyl)carbamoyl)benzoate, which is optionally substituted, to prepare compounds of Formula A1-2 in which in which R¹⁰ is —W—R^{W}, W is —O— and R^{W} is an aromatic ring.

A representative general process for the reaction is as follows. A 40 mL screw top vial is charged with a solution of the chloroquinazoline and the ethyl 4-((5-amino-2-methylphenyl)carbamoyl)benzoate in ethanol. The vial is sealed and heated in an aluminum block (125° C.) for 5 hours. The mixture is cooled and diluted with excess ethanol, then DCM, leaving behind some dark, insoluble material. The extracts are filtered and evaporated to a dark solid. The solid is dissolved in IPA at reflux, then allowed to cool to rt and stir overnight. The precipitated solid product is recovered by filtration.

### Example 10: Process for Preparing Compounds of Formula A1-2 in which R¹⁰ is —W—R^{W}, W is Oxy and R^{W} is an Optionally Substituted Alkyl, Cycloalkyl or Heterocycloalkyl Group

This Example describes a process for preparing a compound of Formula A1-2 in which R¹⁰ is —W—R^{W}, R^{W} is an optionally substituted alkyl, cycloalkyl or optionally substituted heterocycloalkyl, and W is oxy (i.e., —O—), from a substituted quinazoline. A representative intermediate in which R^{W} is cyclohexyl may be prepared by the following general process.

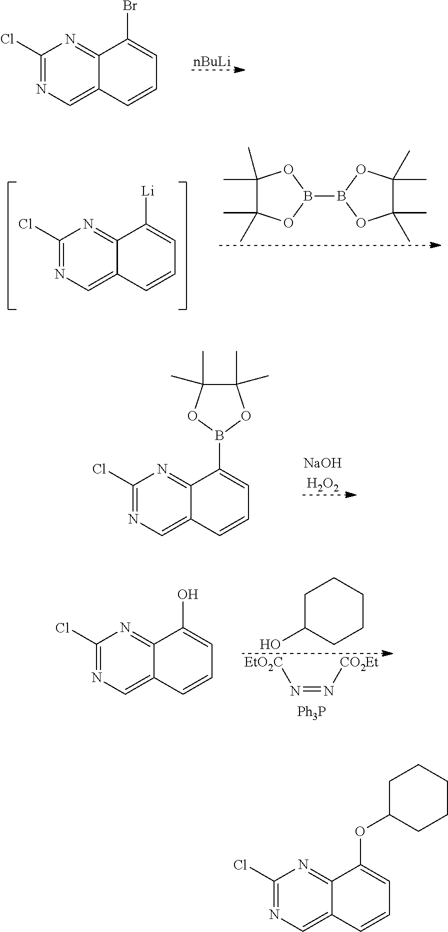

The process includes a lithium-halogen exchange, borylation, a basic H₂O₂ treatment, and Mitsunobu etherification. While a R^{W} cyclohexyl group is shown above, the R^{W} group can be another alkyl, cycloalkyl or heterocycloalkyl, which may be optionally substituted (e.g., an R^{W} group of Formula D2, D3, E2 or E3).

A −78° C. THF solution of the chloro-bromo quinazoline is treated with n-butyllithium followed by addition of a THF solution of bis(pinacolato)diboron. The reaction is allowed to slowly warm to rt, then quenched with a solution of 10% acetic acid in THF. The reaction mixture is diluted with excess iPrOAc and washed with water. The organic phase is recovered, dried over MgSO₄, filtered and evaporated.

The crude boronate is dissolved in EtOH and treated with an aqueous solution of NaO₂H (prepared from NaOH and 30% H₂O₂) at rt. After stirring for an appropriate time the reaction is adjusted to pH 3 with 6N HCl and extracted with excess iPrOAc. The organic phase is dried over MgSO₄, filtered and evaporated to yield the hydroxy-quinazoline which is purified by flash chromatography.

The hydroxy-quinazoline is dissolved in dry THF and sequentially treated at rt with Ph₃P, cyclohexanol and diethyldiazodicarboxylate. After stirring for an appropriate time the reaction is quenched with water and extracted with iPrOAc. The organic phase is dried over MgSO₄, filtered and evaporated. The crude ethereal quinazoline is purified by flash chromatography and used in the process.

The derived 8-cyclohexyloxy-2-chloroquinazoline is reacted with ethyl 4-((5-amino-2-methylphenyl)carbamoyl)benzoate, which is optionally substituted, to prepare compounds of Formula A2 in which W is O and R¹⁰ is a saturated ring.

A representative general process for the reaction is as follows. A 40 mL screw top vial is charged with a solution of the chloroquinazoline and the ethyl 4-((5-amino-2-methylphenyl)carbamoyl)benzoate in ethanol. The vial is sealed and heated in an aluminum block (125° C.) for 5 hours. The mixture is cooled and diluted with excess ethanol, then DCM, leaving behind some dark, insoluble material. The extracts are filtered and evaporated to a dark solid. The solid is dissolved in IPA at reflux, then allowed to cool to rt and stir overnight. The precipitated solid product is recovered by filtration.

Additional information pertaining to the process is in Patent Application Publication No. WO2020257487 published on Dec. 24, 2020.

### Example 11: Process for Preparing Compounds of Formula A1-2 in which R¹⁰ is —W—R^{W} and W is an Alkylene Group

This Example describes a process for preparing a compound of Formula A1-2 in which R¹⁰ is —W—R^{W}, R^{W} is an optionally substituted aryl or optionally substituted heteroaryl, and W is an alkylene group (e.g., —CHCH—), by conversion of a compound of Formula A1-2 having a R¹⁰ bromo group (e.g., Cmpd13, Cmpd14, Cmpd15, Cmpd16, Cmpd17, Cmpd18). A representative compound in which R^{W} is phenyl may be prepared:

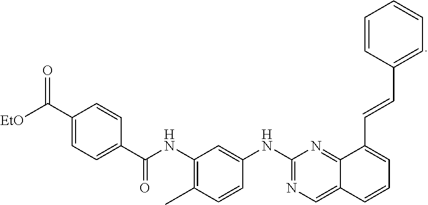 [SMILES: CCOC(=O)c1ccc(C(=O)Nc2cc(Nc3ncc4cccc(/C=C/c5ccccc5)c4n3)ccc2C)cc1]

While a R^{W} phenyl group is shown above, the R^{W} group can be another aryl or heteroaryl group, which may be optionally substituted (e.g., of Formula B2, B3, C2 or C3).

The following process may be implemented. A sealable reaction vessel is charged with a rt solution of Pd(OAc)₂ (0.2 eq with respect to the bromo-quinazoline (see below)) in dry, degassed DMF. The solution is treated with solid Ph₃P (3 eq with respect to Pd(OAc)₂) and stirred until homogenous.

In a separate vessel the bromo-quinazoline (i.e., a compound of Formula A1-2 having a R¹ bromo group (e.g., Cmpd13, Cmpd14, Cmpd15, Cmpd16, Cmpd17, Cmpd18)) in dry, degassed DMF (0.2 M) is treated with styrene (1.5 eq with respect to the bromo-quinazoline), then Et₃N (2.0 eq with respect to the bromo-quinazoline). The bromo-quinazoline solution is added to the Pd(OAc)₂ solution by canula, sealed and heated (to about 100° C.) with monitoring of the reaction by LCMS. Upon completion, the reaction is diluted with excess EtOAc and filtered through Celite. The filtrate is evaporated to a residue that is purified by flash chromatography to afford the desired product.

### Example 12: Preparation of Compounds of Formula A1 in which R¹⁰ is Alkyl, Cycloalkyl or Heterocycloalkyl

This Example describes a process for preparing a compound of Formula A1 having an optionally substituted cycloalkyl or optionally substituted heterocycloalkyl R¹⁰ group. A representative intermediate in which R¹⁰ is cyclohexyl may be prepared by the following general process.

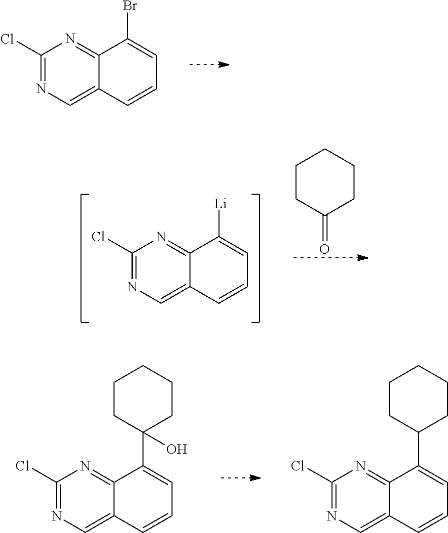

While a R¹⁰ cyclohexyl group is shown above, the R¹⁰ group can be another cycloalkyl or heterocycloalkyl, which may be optionally substituted (e.g., of Formula D2, D3, E2 or E3). The chloro-bromo quinazoline is dissolved in THF and cooled to −78° C. in a dry ice/IPA bath. A solution of n-butyllithium in hexane is added and stirred at low temperature for about 30 min. A solution of cyclohexanone in THF is added at low temperature, stirred for 10 min and then allowed to slowly warm to rt. The reaction is quenched with saturated aqueous sodium bicarbonate and diluted with excess EtOAc. The organic phase is recovered and dried over MgSO₄, filtered, then evaporated to a residue. The residue is purified by flash chromatography.

The derived tertiary alcohol is dissolved in DCM and treated with triethylsilane. The solution is cooled to −10° C. and treated dropwise with TFA until the reaction mixture is 15% TFA by volume. The reaction is allowed to warm to rt and stirred for 1 hr. Toluene is added and the reaction evaporated to a residue. The residue is purified by flash chromatography to afford the 8-cyclohexyl-2-chloroquinazoline product that is then used in the previously described process.

The derived 8-cyclohexyl-2-chloroquinazoline is reacted with ethyl 4-((5-amino-2-methylphenyl)carbamoyl)benzoate, which is optionally substituted, to prepare compounds of Formula A2 in which W is null and R¹⁰ is a saturated ring.

A representative general process for the reaction is as follows. A 40 mL screw top vial is charged with a solution of the chloroquinazoline and the ethyl 4-((5-amino-2-methylphenyl)carbamoyl)benzoate in ethanol. The vial is sealed and heated in an aluminum block (125° C.) for 5 hours. The mixture is cooled and diluted with excess ethanol, then DCM, leaving behind some dark, insoluble material. The extracts are filtered and evaporated to a dark solid. The solid is dissolved in IPA at reflux, then allowed to cool to rt and stir overnight. The precipitated solid product is recovered by filtration.

### Example 13: Preparation of ethyl 4-[[5-[(8-bromoquinazolin-2-yl)amino]-2-methyl-phenyl]carbamoyl]benzoate

Described in this Example is a process for preparing ethyl 4-[[5-[(8-bromoquinazolin-2-yl)amino]-2-methyl-phenyl]carbamoyl]benzoate (Cmpd13), which may be utilized to prepare compounds described herein.

### Preparation of ethyl 4-[[5-(tert-butoxycarbonylamino)-2-methyl-phenyl]carbamoyl]benzoate

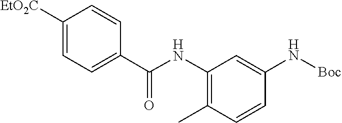 [SMILES: CCOC(=O)c1ccc(C(=O)Nc2cc(NC(=O)OC(C)(C)C)ccc2C)cc1]

A 40 mL screw top vial was charged with 4-ethoxycarbonylbenzoic acid (1.42 g, 7.29 mmol). DMF (5 mL) was added and the mixture stirred until dissolution was complete. The vial was next charged with tert-butyl N-(3-amino-4-methyl-phenyl)carbamate (900 mg, 4.05 mmol) with rinsing using DMF (5 mL). TEA (1.0 mL, 7.29 mmol) was added. The vial was charged with EEDQ (1.80 g, 7.29 mmol) with rinsing using DMF (5 mL). The vial was sealed and stirred at room temperature (rt) for 24 hours (hr).

LCMS showed the reaction was complete. The reaction was poured into excess iPrOAc and washed with saturated aqueous sodium bicarbonate (3×), then water (1×), then 1N HCl (3×), and finally water. The organic phase was dried over MgSO₄, filtered and evaporated to a pale yellow foam (1.192 grams). Attempted redissolution of the foam in EtOAc gives a white suspension of apparent solid. However, no filtration was carried out.

The product was used without further purification.

### Preparation of ethyl 4-[(5-amino-2-methyl-phenyl)carbamoyl]benzoate

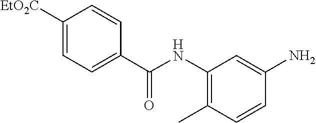 [SMILES: CCOC(=O)c1ccc(C(=O)Nc2cc(N)ccc2C)cc1]

A 40 mL screw top vial was charged with a solution of ethyl 4-[[5-(tert-butoxycarbonylamino)-2-methyl-phenyl]carbamoyl]benzoate (325 mg, 0.82 mmol) in DCM (10 mL). The solution was treated with TFA (2 mL) at rt. The homogenous yellow reaction was stirred for 1 hr and sampled for LCMS. The reaction was complete.

Toluene (10 mL) was added and the solution evaporated to a residue. The residue was partitioned between iPrOAc and saturated aqueous sodium bicarbonate (2× washes). The organic phase was dried over MgSO₄, filtered and evaporated to a yellow oil (238 mg) which becomes a tacky, cream colored solid on standing. The material was used without further purification.

### Preparation of ethyl 4-[[5-[(8-bromoquinazolin-2-yl)amino]-2-methyl-phenyl]carbamoyl]benzoate

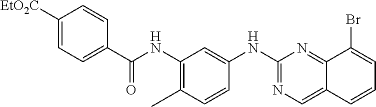 [SMILES: CCOC(=O)c1ccc(C(=O)Nc2cc(Nc3ncc4cccc(Br)c4n3)ccc2C)cc1]

Three 0.5-2.0 mL tapered microwave vials were each charged with ethyl 4-[(5-amino-2-methyl-phenyl)carbamoyl]benzoate (156 mg each, 0.523 mmol). Each vial was then charged with 8-bromo-2-chloro-quinazoline (637 mg, 2.615 mmol). EtOH (2.5 mL) was added to each and the combined solids were agitated using a pipette before sealing. The mixtures were placed in a 130° C. aluminum block and vigorously stirred for 5 hours.

The dark but transparent red-brown solutions were allowed to stand at rt over the weekend. A black precipitate was observed in each vial. The dark supernatants were sampled for LCMS. All three were identical, showing the complete consumption of the aniline.

The three vials were rinsed with excess EtOH, then excess DCM, into a 250 mL round bottom (rb) flask, leaving behind some black insoluble material. The solution was evaporated to an orange-brown oil. IPA (10 mL) was added and the mixture heated until the residue dissolved. The dark solution was allowed to cool and stir at rt for 2 hr. The dark brown precipitated solid was recovered by filtration.

The dark solid in the funnel was dissolved in excess DCM and filtered into a separate rb flask.

The black solid remaining in the reaction vials was layered with excess DCM and stirred at rt for an hour, giving an orange-green supernatant. The operation was repeated three more times with the supernatants being collectively recovered. This third mixture was filtered, leaving behind a green solid in the funnel.

There were four isolated materials. The DCM soluble material from the filter funnel, the first IPA filtrate, the DCM filtrate from the insoluble solids and the final solid left behind after all the solvent treatment.

The DCM soluble material from the funnel yielded a dark brown semi-solid that solidified on standing (264 mg). The material was put through a solid phase extraction (SPE) process using 1 gram silica gel and 15 mL of 50:1 DCM/MeOH. This process yielded an orange solid (220 mg).

The IPA filtrate yielded an orange-brown brittle foam (2.042 g). The material was put through a SPE process using 8 grams of silica gel and 80 mL of 50:1 DCM/MeOH. This process yielded an orange foam (1.037 g).

The DCM filtrate from the insoluble solids yielded a yellow solid (122 mg) judged to be usable as is.

TLC of the SPE process materials showed removal of baseline material. The two materials were chromatographed on normal phase (0% to 5% MeOH/DCM). Some impure product was obtained (120 mg).

The green insoluble material dissolved in DMSO. LCMS showed it was mostly product usable as is (155 mg). Total usable product isolated was 277 mg.

### Example 14: Preparation of ethyl 4-((3-((8-bromoquinazolin-2-yl)amino)-5-methylphenyl)carbamoyl)benzoate

Described in this Example is a process for preparing ethyl 4-((3-((8-bromoquinazolin-2-yl)amino)-5-methylphenyl)carbamoyl)benzoate, Cmpd14 shown in Table A, which may be utilized to prepare compounds described herein.

### Preparation of ethyl 4-((3-((tert-butoxycarbonyl)amino)-5-methylphenyl)carbamoyl)benzoate

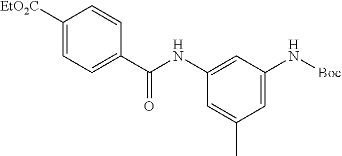 [SMILES: CCOC(=O)c1ccc(C(=O)Nc2cc(C)cc(NC(=O)OC(C)(C)C)c2)cc1]

A 20 mL scintillation vial was charged with 4-ethoxycarbonylbenzoic acid (398 mg, 2.05 mmol). DMF (1.5 mL) was added and the mixture stirred until dissolution was complete. The vial was next charged with tert-butyl N-(3-amino-5-methyl-phenyl)carbamate (253 mg, 1.14 mmol) with rinsing using DMF (1.5 mL). TEA (284 mL, 2.05 mmol) was added. The vial was charged with EEDQ (507 mg, 2.05 mmol) with rinsing using DMF (1.5 mL). The vial was sealed and stirred at rt for 24 hr.

LCMS showed the reaction was complete. The reaction was poured into excess iPrOAc and washed with saturated aqueous sodium bicarbonate (3×), then water (1×), then 1N HCl (3×), and finally water. The organic layer was dried over MgSO₄, filtered, and evaporated to a brown oil (169.5 mg).

The material was used without further purification.

### Preparation of ethyl 4-((3-amino-5-methylphenyl)carbamoyl)benzoate

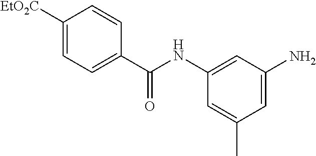 [SMILES: CCOC(=O)c1ccc(C(=O)Nc2cc(C)cc(N)c2)cc1]

A solution of ethyl 4-[[3-(tert-butoxycarbonylamino)-5-methyl-phenyl]carbamoyl]benzoate (169.5 mg, 0.43 mmol) in DCM (1.70 mL) was treated with TFA (430 mL) at room temperature. The homogenous yellow reaction was stirred for 1 hr and sampled for LCMS. The reaction went to completion.

Toluene (10 mL) was added and the solution evaporated to a residue. The residue was partitioned between iPrOAc and saturated aqueous sodium bicarbonate (2× washes). The organic layer was dried over MgSO₄, filtered, and evaporated to a yellow oil (116 mg). The material was used without further purification.

### Preparation of ethyl 4-(3-((8-bromoquinazolin-2-yl)amino)-5-methylphenyl)carbamoyl)benzoate

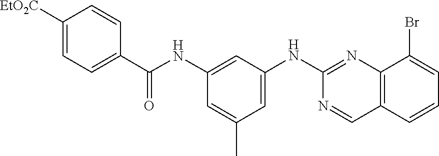 [SMILES: CCOC(=O)c1ccc(C(=O)Nc2cc(C)cc(Nc3ncc4cccc(Br)c4n3)c2)cc1]

Two 0.5-2.0 mL tapered microwave vials were each charged with ethyl 4-((3-amino-5-methylphenyl)carbamoyl)benzoate (58 mg, 0.19 mmol; each batch). Each vial was then charged with 8-bromo-2-chloro-quinazoline (237 mg, 0.97 mmol; each batch). EtOH (3 mL each) was added, and the combined solids were agitated using a pipette before sealing.

The mixtures were heated to 130° C. in an aluminum block, and vigorously stirred for 5 hours.

The two vials were rinsed with excess EtOH, then excess DCM, into a 250 mL round-bottomed flask. The solution was evaporated to an orange-brown oil. IPA (10 mL) was added, and the mixture heated until the residue dissolved. The dark solution was allowed to cool and stir at room temperature for 12 hr. The dark brown precipitated solid was recovered by filtration (83.1 mg).

### Example 15: Preparation of ethyl 4-((5-((8-bromoquinazolin-2-yl)amino)-2-methoxyphenyl)carbamoyl)benzoate

Described in this Example is a process for preparing ethyl 4-((5-((8-bromoquinazolin-2-yl)amino)-2-methoxyphenyl)carbamoyl)benzoate, Cmpd15 shown in Table A, which may be utilized to prepare compounds described herein.

### Preparation of ethyl 4-((5-((tert-butoxycarbonyl)amino)-2-methoxyphenyl)carbamoyl)benzoate

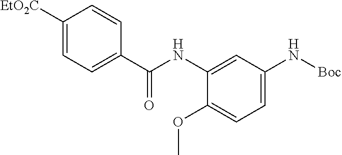 [SMILES: CCOC(=O)c1ccc(C(=O)Nc2cc(NC(=O)OC(C)(C)C)ccc2OC)cc1]

A 20 mL scintillation vial was charged with 4-ethoxycarbonylbenzoic acid (733 mg, 3.78 mmol). DMF (2.8 mL) was added and the mixture stirred until dissolution was complete. The vial was next charged with tert-butyl N-(3-amino-4-methoxy-phenyl)carbamate (500 mg, 2.10 mmol) with rinsing using DMF (2.8 mL). TEA (524 mL, 3.78 mmol) was added. The vial was charged with EEDQ (934 mg, 3.78 mmol) with rinsing using DMF (2.8 mL). The vial was sealed and stirred at rt for 24 hr.

LCMS showed the reaction was complete. The reaction was poured into excess iPrOAc and washed with saturated aqueous sodium bicarbonate (3×), then water (1×), then 1N HCl (3×), and finally water. The organic layer was dried over MgSO₄, filtered, and evaporated to a yellow oil (304 mg).

The material was used without further purification.

### Preparation of ethyl 4-((5-amino-2-methoxyphenyl)carbamoyl)benzoate

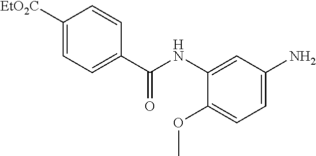 [SMILES: CCOC(=O)c1ccc(C(=O)Nc2cc(N)ccc2OC)cc1]

A solution of ethyl 4-((5-((tert-butoxycarbonyl)amino)-2-methoxyphenyl)carbamoyl)benzoate (304 mg, 0.73 mmol) in DCM (2.9 mL) was treated with TFA (740 mL) at rt. The homogenous yellow reaction was stirred for 1 hr and sampled for LCMS. The reaction went to completion.

Toluene (10 mL) was added and the solution evaporated to a residue. The residue was partitioned between iPrOAc and saturated aqueous sodium bicarbonate (2× washes). The organic layer was dried over MgSO₄, filtered, and evaporated to a yellow oil (224 mg). The material was used without further purification.

### Preparation of ethyl 4-((5-((8-bromoquinazolin-2-yl)amino)-2-methoxyphenyl)carbamoyl)benzoate

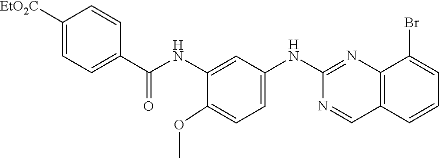 [SMILES: CCOC(=O)c1ccc(C(=O)Nc2cc(Nc3ncc4cccc(Br)c4n3)ccc2OC)cc1]

Two 0.5-2.0 mL tapered microwave vials were each charged with ethyl 4-((5-amino-2-methoxyphenyl)carbamoyl)benzoate (112 mg, 0.375 mmol; each batch). Each vial was then charged with 8-bromo-2-chloro-quinazoline (433 mg, 1.78 mmol; each batch). EtOH (2.5 mL each) was added, and the combined solids were agitated using a pipette before sealing. The mixtures were placed in a 130° C. aluminum block and vigorously stirred for 5 hours.

The two vials were rinsed with excess EtOH, then excess DCM, into a 250 mL round-bottomed (rb) flask. The solution was evaporated to an orange-brown oil. IPA (10 mL) was added and the mixture heated until the residue dissolved. The dark solution was allowed to cool and stir at room temperature for 12 hr. The dark brown precipitated solid was recovered by filtration (126 mg).

### Example 16: Preparation of ethyl 4-((3-((8-bromoquinazolin-2-yl)amino)phenyl)carbamoyl)benzoate

Described in this Example is a process for preparing ethyl 4-((3-((8-bromoquinazolin-2-yl)amino)phenyl)carbamoyl)benzoate, Cmpd16 shown in Table A, which may be utilized to prepare compounds described herein.

### Preparation of ethyl 4-((3-((tert-butoxycarbonyl)amino)phenyl)carbamoyl)benzoate

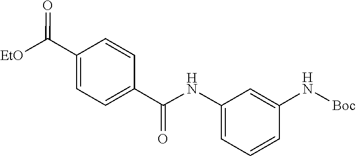 [SMILES: CCOC(=O)c1ccc(C(=O)Nc2cccc(NC(=O)OC(C)(C)C)c2)cc1]

A 20 mL scintillation vial was charged with 4-ethoxycarbonylbenzoic acid (196 mg, 1.009 mmol) followed by tert-butyl N-(3-aminophenyl)carbamate (116 mg, 0.557 mmol). DMF (2 mL) was added and the mixture stirred until dissolution was complete, giving an opaque black solution. TEA (140 μL, 1.003 mmol) was added followed by EEDQ (248 mg, 1.009 mmol). The dark solution was stirred at rt overnight.

LCMS showed a major product with the desired mass. The reaction was diluted with excess iPrOAc and washed with saturated aqueous sodium bicarbonate (3×), water, 1N HCl (3×) and then water. The brown organic was dried over MgSO₄, filtered and evaporated to a dark oil that became a tan solid upon standing under high vacuum (116 mg). The material was used without further purification.

### Preparation of ethyl 4-((3-aminophenyl)carbamoyl)benzoate

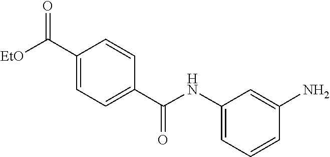 [SMILES: CCOC(=O)c1ccc(C(=O)Nc2cccc(N)c2)cc1]

A solution of ethyl 4-[[5-(tert-butoxycarbonylamino)-phenyl]carbamoyl]benzoate (116 mg, 0.302 mmol) in DCM (3 mL) was treated with TFA (0.6 mL) at rt. The homogenous yellow reaction was stirred for 1 hr and sampled for LCMS. The reaction was determined as being complete.

Toluene (4 mL) was added and the solution evaporated to a residue. The residue was partitioned between iPrOAc and saturated aqueous sodium bicarbonate (2× washes). The organic phase was dried over MgSO₄, filtered and evaporated to a yellow oil which became a tacky, cream colored solid on standing. The material was used without further purification (61 mg).

### Preparation of ethyl 4-((3-((8-bromoquinazolin-2-yl)amino)phenyl)carbamoyl)benzoate

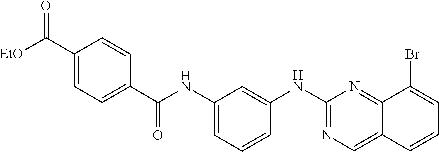 [SMILES: CCOC(=O)c1ccc(C(=O)Nc2cccc(Nc3ncc4cccc(Br)c4n3)c2)cc1]

A 0.5-2.0 mL tapered microwavable vial was charged with a solution of ethyl 4-[3-aminophenyl)carbamoyl]benzoate (61 mg, 0.21 mmol) in EtOH (2 mL). Solid 8-bromo-2-chloro-quinazoline (261 mg, 1.07 mmol) was added. The solution was sealed in a vial and heated to 125° C. in an aluminum block for 5 hours.

LCMS showed that all of the aniline is consumed and the desired product was present. Upon cooling the solution a green suspension was formed. It was rinsed with excess EtOH, and then with excess DCM into a 250 mL rb flask, which left behind a dark insoluble material. The solution was evaporated to an orange oil. IPA (3 mL) was added and the mixture was heated up to dissolve the material. Upon cooling and stirring at rt for 2 hr, the precipitated solid was recovered by filtration.

The solid was dissolved in excess DCM and filtered into a separate rb flask.

The solid left in the microwavable vial was layered with excess DCM and stirred at rt for a hour yielding an orange supernatant. This operation was repeated three more times with the supernatants being collectively recovered.

The DCM solutions were combined to afford the product (14 mg).

### Example 17: Protein Kinase Inhibition Assay and Results

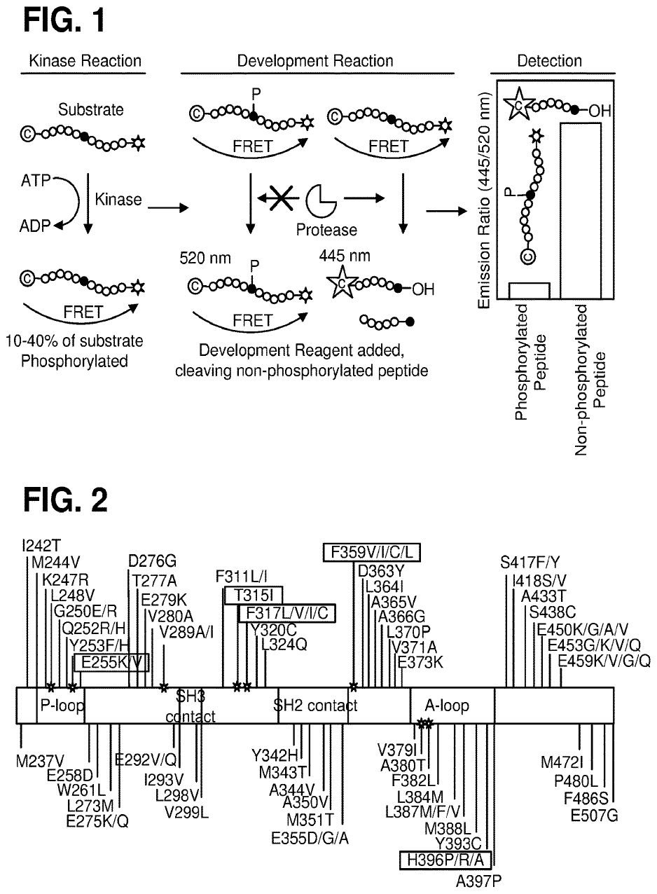

This Example describes a cell-free assay utilized to identify candidate compounds that bind to and inhibit protein kinase (PK) and PK variants. One assay is referred to as a “Z′-LYTE assay” and is an example of a labeled peptide cleavage assay. The fluorescence-based assay relies on the differential sensitivity of a small, dual-end-labelled peptide to cleavage by a protease, dependent on the phosphorylation state of the peptide (FIG. 1). The peptide substrate is end-labelled with two distinct fluorophores that comprise a Fluorescence Resonance Energy Transfer (FRET) pair.

In the absence of inhibitor, the kinase transfers the gamma-phosphate from ATP to the single, unique tyrosine, serine or threonine in the synthetic FRET peptide. The reaction conditions are titrated such that 10-40% of the peptide substrate is phosphorylated. In a second reaction, a site-specific protease that only recognizes and cleaves the non-phosphorylated FRET peptide is added. Cleavage interferes with FRET between the donor (i.e., coumarin) and the acceptor (i.e., fluorescein) fluorophores on the FRET-peptide substrate, unlike the phosphorylated, uncleaved peptide.

To quantitate phosphorylation activity of the kinase, a ratiometric method is used, which is based on the ratio (Emission Ratio (ER)) of donor emission to acceptor emission after excitation of the donor coumarin at 400 nm, using the formula:

ER=Coumarin Emission (445 nm)/Fluorescein Emission (520 nm).

In this approach, both cleaved and uncleaved FRET-peptides contribute to the two fluorescence signals and hence to the ER. Based on the ER, the extent of phosphorylation of the FRET-peptide can be calculated. If the FRET-peptide is phosphorylated (i.e., no or less kinase-mediated phosphorylation inhibition), the ER is relatively low, but if kinase-mediated phosphorylation is inhibited, the ER is relatively high. This ratiometric approach to quantitating reaction progress virtually eliminates well-to-well variations in FRET-peptide concentration and signal intensities, leading to very high Z′-factor values (>0.7; World Wide Web URL en.wikipedia.org/wiki/Z-factor) at a low percent phosphorylation.

Assay regents and conditions are described for an ABL1 PK assay, and are described thereafter for other PK assays.

### Assay Conditions

### Test Compounds

The Test Compounds are screened in 1% DMSO (final) in the well. For 10-point titrations, 3-fold serial dilutions are conducted from a relatively high starting concentration (e.g., 10 micromolar (μM)), considered to be much greater than an IC₅₀ of likely pharmaceutical value.

### ABL1 PK Assay Reagents

All Peptide/Kinase Mixtures are diluted to a 2× working concentration in the appropriate Kinase Buffer. The 2×ABL1 polypeptide/Tyr 02 substrate peptide mixture is prepared in 50 mM HEPES pH 7.5, 0.01% BRIJ-35, 10 mM MgCl₂, 1 mM EGTA. The final 10 μL Kinase Reaction contains 0.3-1.2 ng ABL1 (or 1.36-6 ng ABL1(T315I)) and 2 μM Tyr 02 in 50 mM HEPES, pH 7.5, 0.01% BRIJ-35, 10 mM MgCl₂, 1 mM EGTA. After the 1-hour Kinase Reaction incubation, 5 μL of a 1:128 dilution of Development Reagent A is added. The ABL1(wt) polypeptide utilized for the assay described in this Example was the “isoahABL1(wt)” polypeptide described herein (SEQ ID NO:4), which is referred to as “ABL1” in Table 1 and “ABL1(wt)” or “WT” in Table 3 of this Example. The ABL1 variant polypeptides utilized contained the same polypeptide as the “isoahABL1(wt)” polypeptide except that each contained one corresponding amino acid substitution chosen from E255K, F317I, F317L, G250E, T315I or Y253F. The ABL1 variant polypeptides are referred to as “ABL1 E255K,” “ABL1 F317I,” “ABL1 F317L,” “ABL1 G250E,” “ABL1 T315I” and “ABL1 Y253F” in Table 1 of this Example according to the corresponding amino acid substitution contained and are referred to in Table 3 of this Example according to the corresponding amino acid substitution contained.

All ATP Solutions are diluted to a 4× working concentration in Kinase Buffer (50 mM HEPES, pH 7.5, 0.01% BRIJ-35, 10 mM MgCl₂, 1 mM EGTA). The ATP Km apparent (Km app) is previously determined using a Z′-LYTE assay. The following Table 1 in this Example shows IC₅₀ values for particular inhibitors of phosphorylation activity for ABL1(wt) polypeptide and ABL1 variant polypeptides.

TABLE 1

| | | ATP | ATP | | |
|---|---|---|---|---|---|
| | Z′-LYTE | Km app | Bin | | IC50 |
| Assay | Substrate | (μM) | (μM) | Inhibitor | (nM) |
| ABL1 | Tyr 02 | 12 | 10 | Tyrphostin AG1478 | 326 |
| ABL1 E255K | Tyr 02 | 4.2 | 5 | Staurosporine | 71.1 |
| ABL1 F317I | Tyr 02 | 15 | 10 | Staurosporine | 205 |
| ABL1 F317L | Tyr 02 | 14 | 10 | Staurosporine | 214 |
| ABL1 G250E | Tyr 02 | 5.4 | 5 | Staurosporine | 29.9 |
| ABL1 T315I | Tyr 02 | 2.8 | 5 | Staurosporine | 12.3 |
| ABL1 Y253F | Tyr 02 | 11.8 | 10 | Staurosporine | 44.1 |

The Development Reagent (i.e., with protease) is diluted in Development Buffer.

### Assay Protocol

Bar-coded Corning, low volume NBS, black 384-well plate (Corning Cat. #4514)
1. 100 nL—100× Test Compound in 100% DMSO
2. 2.4 μL—Kinase buffer
3. 5 μL—2× Peptide/Kinase Mixture
4. 2.5 μL—4×ATP Solution
5. 30-second plate shake
6. 60-minute Kinase Reaction incubation at room temperature
7. 5 μL—Development Reagent Solution
8. 30-second plate shake
9. 60-minute Development Reaction incubation at room temperature
10. Read on fluorescence plate reader and analyze the data

### Z′-LYTE Assay Controls

The following controls are made for each individual kinase and are located on the same plate as the kinase.

### 0% Phosphorylation Control (100% Inhibition Control)

The maximum Emission Ratio is established by the 0% Phosphorylation Control (100% Inhibition Control), which contains no ATP and therefore exhibits no kinase activity. This control yields 100% cleaved peptide in the Development Reaction.

### 100% Phosphorylation Control

The 100% Phosphorylation Control, which contains a synthetically phosphorylated peptide of the same sequence as the peptide substrate, is designed to allow for the calculation of percent phosphorylation. This control yields a very low percentage of cleaved peptide in the Development Reaction.

The 0% Phosphorylation and 100% Phosphorylation Controls allow one to calculate the percent Phosphorylation achieved in a specific reaction well. Control wells do not include any kinase inhibitors.

### 0% Inhibition Control

The minimum Emission Ratio in a screen is established by the 0% Inhibition Control, which contains active kinase. This control is designed to produce a 10-50% phosphorylated peptide in the Kinase Reaction. Cascade assays may produce up to 70% phosphorylated peptide.

### Known Inhibitor

A known inhibitor control standard curve, 10-point titration, is run for each individual kinase on the same plate as the kinase to ensure the kinase is inhibited within an expected IC₅₀ range previously determined.

The following controls are prepared for each concentration of Test Compound assayed:

### Development Reaction Interference

The Development Reaction Interference is established by comparing the Test Compound Control wells that do not contain ATP versus the 0% Phosphorylation Control (which does not contain the Test Compound). The expected value for a non-interfering compound should be 100%. Any value outside of 90% to 110% is flagged.

### Test Compound Fluorescence Interference

The Test Compound Fluorescence Interference is determined by comparing the Test Compound Control wells that do not contain the Kinase/Peptide Mixture (zero peptide control) versus the 0% Inhibition Control. The expected value for a non-fluorescence compound should be 0%. Any value >20% is flagged.

### Graphing Software

SelectScreen Kinase Profiling Service uses XLfitfrom IDBS. The dose response curve is curve fit to model number 205 (sigmoidal dose-response model). If the bottom of the curve does not fit between −20% & 20% inhibition, it is set to 0% inhibition. If the top of the curve does not fit between 70% and 130% inhibition it is set to 100% inhibition.

### Z′-LYTE Data Analysis

The equations shown in the following Table 2 of this Example are used for each set of data points:

TABLE 2

| | Equation |
|---|---|
| Correction for Background Fluorescence | FI_{Sample} − FI [symbol] |
| Emission Ratio (using values convicted for florescence) | $\frac{Coumarin Emission ( 445 nm )}{Fluorescein Emission ( 520 nm )}$ |
| % Phosphorylation (% Phos) | $\{ 1 - \frac{( Emission Ratio \times \text{?} ) - \text{?}}{( \text{?} - \text{?} ) + [ Emission Ratio \times ( \text{?} - \text{?} ) ]} \} * 100$ |
| % Inhibition | $\{ 1 - \frac{\% \text{?}}{\% \text{?}} \} * 100$ |
| Z′ (using Emission Ratio values) | $1 - \frac{3 \text{?} + 3 \text{?}}{Mean \text{?} \cdot Mean \text{?}}$ |
| Difference Between Data Points | \| % Inhibition [symbol] · % Inhibition [symbol] \| |
| (single point only) | |
| Development Reaction Interference (DRI) (no ATP control) | $\frac{Emission Ratio \text{?}}{Emission Ratio \text{?}}$ |
| Test Compound Fluorescence Interference (TCFI) | $\frac{FI \text{?}}{FI \text{?}}$ |
| (check both Coumarin and Fluorescein emissions) | |

FI = Fluorescence Intensity
C_{100%} = Average Coumarin emission signal of the 100% Phos. Control
C_{0%} = Average Coumarin emission signal of the 0% Phos. Control
F_{100%} = Average Fluorescein emission signal of the 100% Phos. Control
F_{0%} = Average Fluorescein emission signal of the 0% Phos. Control
DRI = Development Reaction Interference
TCFI = Test Compound Fluorescence Interference [symbol]
indicates data missing or illegible when filed

### DOCUMENTS

The description of the assay in this Example is excerpted from (Z′LYTE™ Screening Protocol and Assay Conditions (rev 29 Jan. 2021)).

### Other PK Assays

Assays for PKs other than ABL1 generally followed the ABL1 PK assay format described previously in this Example, although the identity of Substrates and Inhibitor and the amounts of Kinase Enzyme, ATP inhibitors and Dilution Reagent A added were optimized for each PK, as summarized hereafter.

AURKA (Aurora A) and AURKB (Aurora B): The 2×AURKA (Aurora A)/Ser/Thr 01 mixture is prepared in 50 mM HEPES pH 7.5, 0.01% BRIJ-35, 10 mM MgCl₂, 1 mM EGTA. The final 10 μL Kinase Reaction contains 0.75-3 ng AURKA (Aurora A) or 3.5-18 ng AURKB (Aurora B) and 2 μM Ser/Thr 01 in 50 mM HEPES pH 7.5, 0.01% BRIJ-35, 10 mM MgCl₂, 1 mM EGTA. After the 1 hour Kinase Reaction incubation, 5 μL of a 1:4096 dilution of Development Reagent A is added.

AURKC (Aurora C): The 2×AURKC (Aurora C)/Ser/Thr 19 mixture is prepared in 50 mM HEPES pH 7.5, 0.01% BRIJ-35, 10 mM MgCl₂, 1 mM EGTA. The final 10 μL Kinase Reaction contains 2-20 ng AURKC (Aurora C) and 2 μM Ser/Thr 19 in 50 mM HEPES pH 7.5, 0.01% BRIJ-35, 10 mM MgCl₂, 1 mM EGTA. After the 1 hour Kinase Reaction incubation, 5 μL of a 1:256 dilution of Development Reagent A is added.

BTK, BMX and ITK: The 2×BTK/Tyr 01 mixture is prepared in 50 mM HEPES pH 7.5, 0.01% BRIJ-35, 10 mM MgCl2, 1 mM EGTA. The final 10 μL Kinase Reaction contains 0.52-3.36 ng BTK, or 2.5-10 ng BMX, or 4.69-60 ng ITK, and 2 μM Tyr 01 in 50 mM HEPES pH 7.5, 0.01% BRIJ-35, 10 mM MgCl₂, 1 mM EGTA. After the 1 hour Kinase Reaction incubation, 5 μL of a 1:128 dilution of Development Reagent B is added.

JAK1, JAK2 and JAK3: The 2×JAK1/Tyr 06 mixture is prepared in 50 mM HEPES pH 6.5, 0.01% BRIJ-35, 10 mM MgCl₂, 1 mM EGTA, 0.02% NaN₃. The final 10 μL Kinase Reaction contains 22.9-91.5 ng JAK1, or 0.12-0.5 ng JAK2, 1.75-9 ng JAK2 JH1-JH2-V617F, or 0.5-2.7 ng JAK3 and 2 μM Tyr 06 in 50 mM HEPES pH 7.0, 0.01% BRIJ-35, 10 mM MgCl₂, 1 mM EGTA, 0.01% NaN₃. After the 1 hour Kinase Reaction incubation, 5 μL of a 1:128 dilution of Development Reagent A is added.

EPHA1 and EPHB1: The 2×EPHA1/Tyr 02 mixture is prepared in 50 mM HEPES pH 7.5, 0.01% BRIJ-35, 10 mM MgCl2, 1 mM EGTA. The final 10 μL Kinase. The reaction contains 3.12-38.6 ng EPHA1 (or 1.2-4.8 ng EPHA8, or 2.4-10 ng EPHB1, or 0.55-2.86 ng EPHB1) and 2 μM Tyr 02 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent A is added.

EPHA2: The 2×EPHA2/Tyr 01 mixture is prepared in 50 mM HEPES pH 7.5, 0.01% BRIJ-35, 10 mM MgCl₂, 1 mM EGTA. The final 10 μL Kinase Reaction contains 2.1-8.8 ng EPHA2 (or 1.2-7.5 ng EPHA5) and 2 μM Tyr 01 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent B is added.

TRKA (NTRK1) and TRKC (NTRK3): The 2×NTRK1 (TRKA)/Tyr 01 mixture is prepared in 50 mM HEPES pH 7.5, 0.01% BRIJ-35, 10 mM MgCl₂, 1 mM EGTA. The final 10 μL Kinase Reaction contains 6-24 ng NTRK1 (TRKA) or 2.7-30 ng NTRK3 (TRKC), and 2 μM Tyr 01 in 50 mM HEPES pH 7.5, 0.01% BRIJ-35, 10 mM MgCl₂, 1 mM EGTA. After the 1 hour Kinase Reaction incubation, 5 μL of a 1:128 dilution of Development Reagent B is added.

TRKB (NTRK2): The 2×NTRK2 (TRKB)/Tyr 01 mixture is prepared in 50 mM HEPES pH 7.5, 0.01% BRIJ-35, 10 mM MgCl₂, 4 mM MnCl₂, 1 mM EGTA, 2 mM DTT. The final 10 μL Kinase Reaction contains 0.34-4 ng NTRK2 (TRKB) and 2 μM Tyr 01 in 50 mM HEPES pH 7.5, 0.01% BRIJ-35, 10 mM MgCl₂, 2 mM MnCl₂, 1 mM EGTA, 1 mM DTT. After the 1 hour Kinase Reaction incubation, 5 μL of a 1:128 dilution of Development Reagent B is added.

ROS1: The 2×ROS1/Tyr 01 mixture is prepared in 50 mM HEPES pH 7.5, 0.01% BRIJ-35, 10 mM MgCl₂, 1 mM EGTA. The final 10 μL Kinase Reaction contains 3-12 ng ROS1 and 2 μM Tyr 01 in 50 mM HEPES pH 7.5, 0.01% BRIJ-35, 10 mM MgCl₂, 1 mM EGTA. After the 1 hour Kinase Reaction incubation, 5 μL of a 1:128 dilution of Development Reagent B is added.

TNK1: The 2×TNK1/Ser/Thr 13 mixture is prepared in 50 mM HEPES pH 6.5, 0.01% BRIJ-35, 10 mM MgCl₂, 1 mM EGTA, 0.02% NaN₃. The final 10 μL Kinase Reaction contains 15-60 ng TNK1 and 2 μM Ser/Thr 13 in 50 mM HEPES pH 7.0, 0.01% BRIJ-35, 10 mM MgCl₂, 1 mM EGTA, 0.01% NaN₃. After the 1 hour Kinase Reaction incubation, 5 μL of a 1:1024 dilution of Development Reagent A is added.

TXK: The 2×TXK/Tyr 06 mixture is prepared in 50 mM HEPES pH 7.5, 0.01% BRIJ-35, 10 mM MgCl₂, 1 mM EGTA. The final 10 μL Kinase Reaction contains 4.75-19 ng TXK and 2 μM Tyr 06 in 50 mM HEPES pH 7.5, 0.01% BRIJ-35, 10 mM MgCl₂, 1 mM EGTA. After the 1 hour Kinase Reaction incubation, 5 μL of a 1:128 dilution of Development Reagent A is added.

TYK2: The 2×TYK2/Tyr 03 mixture is prepared in 50 mM HEPES pH 6.5, 0.01% BRIJ-35, 10 mM MgCl₂, 1 mM EGTA, 0.02% NaN₃. The final 10 μL Kinase Reaction contains 3.75-15 ng TYK2 and 2 μM Tyr 03 in 50 mM HEPES pH 7.0, 0.01% BRIJ-35, 10 mM MgCl₂, 1 mM EGTA, 0.01% NaN₃. After the 1 hour Kinase Reaction incubation, 5 μL of a 1:4096 dilution of Development Reagent A is added.

RET: The 2×RET/Tyr 02 mixture is prepared in 50 mM HEPES pH 7.5, 0.01% BRIJ-35, 10 mM MgCl₂, 1 mM EGTA. The final 10 μL Kinase Reaction contains 0.49-3.64 ng RET (or 0.52-4.74 ng RET-V804L or 0.86-6.16 ng RET-Y791F) and 2 μM Tyr 02 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent A is added.

RET-A883F: The 2×RET A883F/Tyr 04 mixture is prepared in 50 mM HEPES pH 6.5, 0.01% BRIJ-35, 10 mM MgCl₂, 1 mM EGTA, 0.02% NaN₃. The final 10 μL Kinase Reaction contains 1.02-6.74 ng RET-A883F (or 3-20 ng RET-V804L) and 2 μM Tyr 04 in 50 mM HEPES pH 7.0, 0.01% BRIJ-35, 10 mM MgCl₂, 1 mM EGTA, 0.01% NaN₃. After the 1-hour Kinase Reaction incubation, 5 μL of a 1:64 dilution of Development Reagent B is added.

RET-S891A: The 2×RET S891A/Tyr 06 mixture is prepared in 50 mM HEPES pH 7.5, 0.01% BRIJ-35, 10 mM MgCl₂, 1 mM EGTA. The final 10 μL Kinase Reaction contains 0.3-1.4 ng RET-S891A and 2 μM Tyr 06 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent A is added.

ABL2 (Arg): The 2×ABL2 (Arg)/Tyr 02 mixture is prepared in 50 mM HEPES pH 7.5, 0.01% BRIJ-35, 10 mM MgCl₂, 1 mM EGTA. The final 10 μL Kinase Reaction contains 0.42-3.13 ng ABL2 (Arg) and 2 μM Tyr 02 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent A is added.

PTK2B (FAK2): The 2×PTK2B (FAK2)/Tyr 01 mixture is prepared in 50 mM HEPES pH 7.5, 0.01% BRIJ-35, 10 mM MnCl₂, 1 mM EGTA, 2 mM DTT, 0.02% NaN₃. The final 10 μL Kinase Reaction contains 5.26-34.8 ng PTK2B (FAK2) and 2 μM Tyr 01 in 50 mM HEPES pH 7.5, 0.01% BRIJ-35, 5 mM MgCl₂, 5 mM MnCl₂, 1 mM EGTA, 1 mM DTT, 0.01% NaN₃. After the 1-hour Kinase Reaction incubation, 5 μL of a 1:128 dilution of Development Reagent B is added.

SRC, SRC-N1: The 2×SRC/Tyr 02 mixture is prepared in 50 mM HEPES pH 7.5, 0.01% BRIJ-35, 10 mM MgCl₂, 1 mM EGTA. The final 10 μL Kinase Reaction consists of 5-20 ng SRC (or 1-4.9 ng SRC-N1) and 2 μM Tyr 02 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent A is added.

LCK: The 2×LCK/Tyr 02 mixture is prepared in 50 mM HEPES pH 7.5, 0.01% BRIJ-35, 10 mM MgCl₂, 1 mM EGTA. The final 10 μL Kinase Reaction consists of 8-33 ng LCK and 2 μM Tyr 02 in 50 mM HEPES pH 7.5, 0.01% BRIJ-35, 10 mM MgCl₂, 1 mM EGTA. After the 1-hour Kinase Reaction incubation, 5 μL of a 1:128 dilution of Development Reagent A is added.

The following Table 3 in this Example shows IC₅₀ values for particular inhibitors of phosphorylation activity for PKs other than ABL1.

TABLE 3

| | | ATP | ATP | | |
|---|---|---|---|---|---|
| | Z′-LYTE | Km app | Bin | Ctl | IC₅₀ |
| Assay | Substrate | (uM) | (uM) | Inhibitor | (nM) |
| AURKA | Ser/Thr 01 | 10 | 10 | Staurosporine | 2.84 |
| (Aurora A) | | | | | |
| AURKB | Ser/Thr 01 | 81 | 75 | Staurosporine | 5.04 |
| (Aurora B) | | | | | |
| AURKC | Ser/Thr 19 | 26 | 25 | Staurosporine | 4.33 |
| (Aurora C) | | | | | |
| BMX | Tyr 01 | 107 | 100 | Staurosporine | 15.4 |
| BTK | Tyr 01 | 36 | 25 | Tyrphostin | 1800 |
| | | | | AG1478 | |
| EPHA1 | Tyr02 | 18.5 | 25 | Staurosporine | 60.0 |
| EPHA2 | Tyr01 | 65.4 | 75 | Staurosporine | 202 |
| EPHA5 | Tyr 01 | 129 | 150 | Staurosporine | 60.1 |
| EPHA8 | Tyr 02 | 123 | 100 | Staurosporine | 94.2 |
| EPHB1 | Tyr02 | 61.4 | 50 | Staurosporine | 74.3 |
| EPHB2 | Tyr 02 | 66 | 75 | Staurosporine | 82.7 |
| ITK | Tyr 01 | 5.6 | 5 | Staurosporine | 16.2 |
| TXK | Tyr 06 | 96 | 100 | Staurosporine | 36.7 |
| JAK1 | Tyr 06 | 87 | 75 | Staurosporine | 1.86 |
| JAK2 | Tyr 06 | 31 | 25 | Staurosporine | 0.456 |
| JAK3 | Tyr 06 | 14 | 10 | Staurosporine | 0.8 |
| TYK2 | Tyr 03 | 23 | 25 | Staurosporine | 1.12 |
| NTRK1 | Tyr 01 | 425 | 400 | Staurosporine | 5.78 |
| (TRKA) | | | | | |
| NTRK2 | Tyr 01 | 22 | 25 | Staurosporine | 0.792 |
| (TRKB) | | | | | |
| NTRK3 | Tyr 01 | 54 | 50 | Staurosporine | 1.94 |
| (TRKC) | | | | | |
| TNK1 | Ser/Thr 13 | 237 | 200 | Staurosporine | 2.78 |
| ROS1 | Tyr 01 | 61.4 | 50 | Staurosporine | 2.22 |
| RET | Tyr 02 | 11 | 10 | Staurosporine | 2.89 |
| RET-Y791F | Tyr 02 | 12 | 10 | Staurosporine | 3.12 |
| RET-V804E | Tyr 04 | 50 | 50 | Staurosporine | 2.29 |
| RET-V804L | Tyr 02 | 5 | 5 | Staurosporine | 3.12 |
| RET-A883F | Tyr 04 | 10 | 10 | Staurosporine | 11.6 |
| RET-S891A | Tyr 06 | 8 | 10 | Staurosporine | 0.721 |
| ABL2 | Tyr 02 | 30 | 25 | Tyrphostin | 731 |
| | | | | AG1478 | |
| PTK2B | Tyr 01 | 5 | 5 | Staurosporine | 12.0 |
| (FAK2) | | | | | |
| SRC | Tyr 02 | 50 | 50 | Tyrphostin | 1650 |
| | | | | AG1478 | |
| SRC-N1 | Tyr 02 | 50 | 50 | Tyrphostin | 1050 |
| | | | | AG1478 | |
| LCK | Tyr 02 | 45 | 50 | Tyrphostin | 631 |
| | | | | AG1478 | |

For a subset of targets, a different type of assay was conducted, known as a LanthaScreen™ Eu Kinase Binding Assay, which is an example of a fluorescence displacement assay. In this case, an Alexa Fluor™-conjugated “tracer”, or control binding ligand, is added to an epitope-tagged target protein in solution. At the same time, an Eu (europium)-labeled anti-tag antibody is added, resulting in a high degree of Fluorescence resonance energy transfer (FRET), whereas displacement of the tracer with a kinase inhibitor leads to a reduction of FRET. For more experimental details, see “LanthaScreen™ Eu Kinase Binding Assay Screening Protocol and Assay Conditions” (Revised 29 January-2021) from SelectScreen™ Biochemical Kinase Profiling Service (World Wide Web address URL thermofisher.com/selectscreen).

For example, TEC, IRAK3, PLK4, RET-G691 S, RET-V804M, RET-M918T, TN K2 (ACK), DDR2-N456S and DDR2-T654M PKs were assayed using the Lantha Screen. The TEC PK concentration was 1 nM using an Eu-anti-His antibody (2 nM) and Tracer 178 at 1 nM (Kd=1 nM). The control inhibitor for the TEC PK was dasatinib (IC₅₀=72.6 nM). The Buffer was 50 mM HEPES pH 7.5, 0.01% BRIJ-35, 10 mM MgCl₂, 1 mM EGTA.

The IRAK3 kinase concentration was 1 nM using an Eu-anti-GST antibody (2 nM) and Tracer 236 at 5 nM (Kd=2.7 nM). The control inhibitor was Staurosporine (IC₅₀=0.351 nM). The Buffer was 50 mM HEPES pH 7.5, 0.01% BRIJ-35, 10 mM MgCl₂ and 1 mM EGTA.

The PLK4 kinase concentration was 1 nM using an Eu-anti-GST antibody (2 nM) and Tracer 236 at 1 nM (Kd=1.7 nM). The control inhibitor was Staurosporine (IC₅₀=1.46 nM). The Buffer was 50 mM HEPES pH 7.5, 0.01% BRIJ-35, 10 mM MgCl₂ and 1 mM EGTA.

The RET-G691S, -V804M, and -M918T kinase concentrations were 5, 2.5, and 20 nM, respectively using an Eu-anti-GST antibody (2 nM) and Tracer 236 at 10, 5, and 10 nM, respectively (K_{d}s=12, 2.2, and 11 nM, respectively). The control inhibitor was staurosporine (IC₅₀=3.18, 1.02, and 3.8 nM, respectively). The Buffer was 50 mM HEPES pH 7.5, 0.01% BRIJ-35, 10 mM MgCl₂, 1 mM EGTA.

The TNK2 (ACK) kinase concentration was 5 nM using an Eu-anti-GST antibody (2 nM) and Tracer 236 at 30 nM (Kd=23 nM). The control inhibitor was Staurosporine (IC₅₀=4.07 nM). The Buffer was 50 mM HEPES pH 7.5, 0.01% BRIJ-35, 10 mM MgCl₂, 1 mM EGTA.

The DDR2-N456S kinase concentration was 1 nM using an Eu-anti-GST antibody (2 nM) and Tracer 236 at 1 nM (Kd=1.8 nM). The control inhibitor was Staurosporine (IC₅₀=0.134 nM). The Buffer was 50 mM HEPES pH 7.5, 0.01% BRIJ-35, 10 mM MgCl₂, 1 mM EGTA. The DDR2-T654M kinase concentration was 20 nM using an Eu-anti-GST antibody (2 nM) and Tracer 178 at 100 nM (Kd=166 nM). The control inhibitor was Dasatinib (IC₅₀=139 nM). The Buffer was 50 mM HEPES pH 7.5, 0.01% BRIJ-35, 10 mM MgCl₂, 1 mM EGTA.

For IRAK1, a different type of assay was conducted, known as an Adapta™ Assay based on ADP formation from ATP hydrolysis. It can be used to measure any type of ATP hydrolysis, including the intrinsic ATPase activity of kinases. In this assay, the substrate is water and not a peptide. This assay is described in Kashem, M A et al. (2007) J. Biomol. Screen. 12:70-83. For IRAK1 assay, the 2×IRAK1/Histone H3 (1-20) peptide mixture is prepared in 50 mM HEPES pH 7.5, 0.01% BRIJ-35, 10 mM MgCl₂, 1 mM EGTA. The final 10 μL Kinase Reaction contains 3.5-30.5 ng IRAK1 and 100 μM Histone H3 (1-20) peptide in 32.5 mM HEPES pH 7.5, 0.005% BRIJ-35, 5 mM MgCl₂, 0.5 mM EGTA. After the 1-hour Kinase Reaction incubation, 5 μL of Detection Mix is added. For additional details, see “Adapta™ Screening Protocol and Assay Conditions” (Revised 29 January-2021) from SelectScreen™ Biochemical Kinase Profiling Service (World Wide Web URL thermofisher.com/selectscreen).

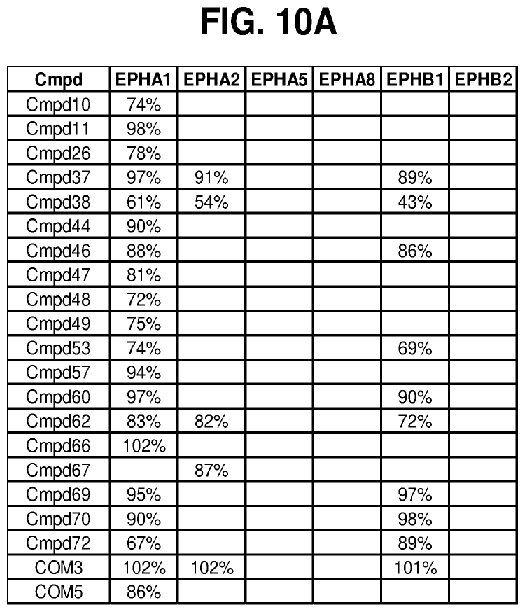

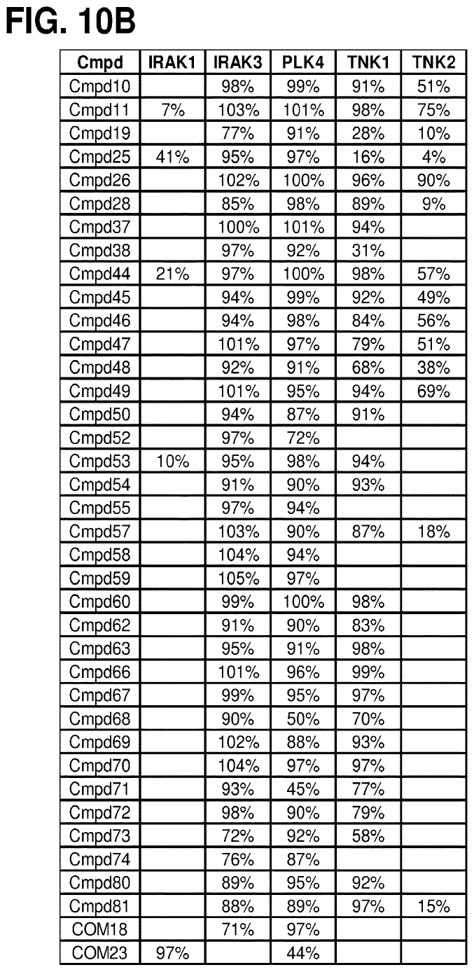

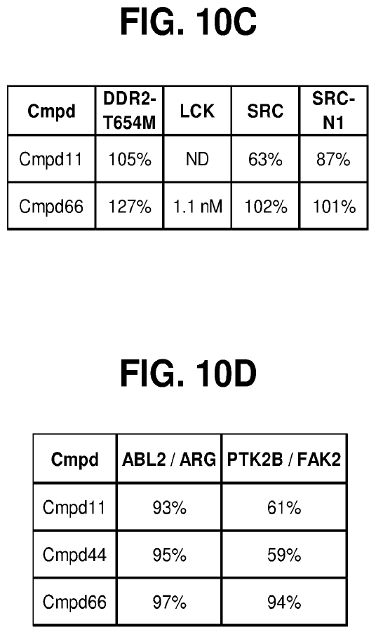

For LCK, the Z′-LYTE labeled peptide cleavage assay described above in this Example can be utilized, as well as a KINOMEscan™ solid phase inhibitor competition assay named “Lck Human TK Kinase Binding SAFETYscan SafetyScreen Assay-US” (EuroFins Discovery, referred to as a “LCK competition assay”). The assay result reported for Cmpd66 in FIG. 10C was obtained using the LCK competition assay.

The following Table 4 in this Example shows public database accession numbers for PK polypeptides (“NP” and “AA” prefix accession numbers in the World Wide Web URL ncbi.nlm.nih.gov/protein/database and “P” prefix accession numbers in the World Wide Web URL uniprot.org/uniprotkb/database), the epitope tag attached and the purity of the PK utilized in each assay.

TABLE 4

| | Accession | | Purity (SDS |
|---|---|---|---|
| PTK* | number* | Epitope | PAGE) |
| ABL1(wt) | NP_005148.2 | 6xHis | 80% |
| BTK(wt) | NP_000052 | 6xHis | 90% |
| BMX(wt) | NP_001712 | 6xHis | 70% |
| ITK(wt) | NP_005537.3 | GST | 80% |
| TEC(wt)\hat{ } | NP_003206 | 6xHis | 85% |
| TXK(wt) | NP_003319 | GST | 90% |
| AURKA(wt) | NP_940839 | 6xHis | 85% |
| AURKB(wt) | NP_004208 | 6xHis | 70% |
| AURKC(wt) | NP_003151 | GST | 90% |
| JAK1(wt) (866-1154) | NP_002218.2 | GST | 75% |
| JAK2(wt) (808-1132) | NP_004963 | GST | 90% |
| JAK3(wt) (781-1124) | NP_000206 | GST | 80% |
| TYK2(wt) (833-1187) | NP_003322.2 | GST | 85% |
| TRKA(wt) (441-796) | NP_002520 | 6xHis | 95% |
| TRKB(wt) (526-838) | NP_006171 | 6xHis | 85% |
| TRKC(wt) (510-825) | NP_002521.2 | 6xHis | 85% |
| ROS1(wt) | NP_002935 | GST | 80% |
| RET(wt) (658-1114)\hat{ }\hat{ } | NP_066124 | GST | 85% |
| EPHA1(wt) (568-976) | NP_005223.2 | GST | 85% |
| EPHA2(wt) (560-976) | NP_004422.2 | GST | 95% |
| EPHA5(wt) (595-1037) | NP_004430.1 | GST | 75% |
| EPHA8(wt) (565-1005) | NP_065387 | GST | 90% |
| EPHB1(wt) (612-887) | NP_004432 | GST | 90% |
| EPHB2 (616-884) | NP_004433 | GST | 90% |
| TNK1(wt) | NP_003976.2 | GST | 70% |
| TNK2 (ACK) (110-476)\hat{ } | NP_005772.3 | GST | 90% |
| PLK4(wt)\hat{ } | NP_055079.3 | GST | 70% |
| IRAK1 (194-712)\hat{ }\hat{ }\hat{ } | NP_001560 | GST | 95% |
| IRAK3(wt)\hat{ } | AAH57800.1 | GST | 80% |
| ABL2(wt) | NP_009298 | 6xHis | 70% |
| PTK2B (FAK2) | NP_005772.3 | GST | 90% |
| DDR2-N456S (422-855) | NP_006173.2 | GST- | ND* |
| | | 6xHis | |
| DDR2-T654M (422-855) | NP_006173.2 | GST- | ND |
| | | 6xHis | |
| SRC(wt) | NP_005408 | 6xHis | 85% |
| | P12931-1 | | |
| SRC-N1(wt) | P12931-2 | none | 80% |
| LCK(wt) | NP_005347 | 6xHis | 70% |

*certain PKs are receptor PKs and the subsequence of the catalytic domain utilized in assays is indicated by a subsequence (in parentheses) of the polypeptide accessed by the corresponding accession number;
\hat{ } LanthaScreen ™ Eu Kinase Binding Assay was utilized;
\hat{ }\hat{ } LanthaScreen ™ Eu Kinase Binding Assay was utilized for certain RET variants;
\hat{ }\hat{ }\hat{ } Adapta ™ Assay was utilized;
Z′LYTE ™ assay utilized for PKs listed in Table 4 not designated by \hat{ }, \hat{ }\hat{ } or \hat{ }\hat{ }\hat{ };
“GST” is glutathione S-transferase conjugate and “6xHis” is a peptide conjugate consisting of six consecutive histidine amino acids.

### Assay Results

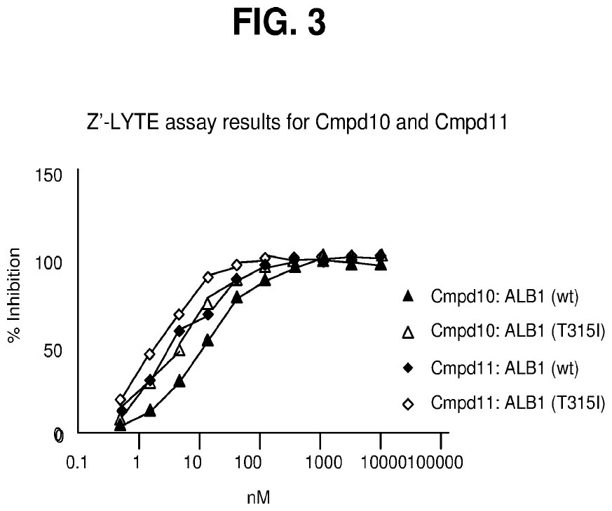

Cmpd10 and Cmpd11 (see e.g., Table A) were assayed as test compounds in the ABL1 Z′LYTE™ assay, as illustrated in FIG. 3. The IC₅₀ value for each test compound measured for ABL1(wt) and ABL1(T315I) variant is reported in nanomolar (nM) units in the following Table 5 of this Example. As defined herein, Cmpd10 and Cmpd11 each is considered an “effective” inhibitor of ABL1(wt) and ABL(T315I) according to IC₅₀ values in Table 5 of this Example.

TABLE 5
IC₅₀ values (nM) for test compounds

| Compound | ABL1(wt) | ABL1(T315I) |
|---|---|---|
| Cmpd10 | 11.2 | 3.86 |
| Cmpd11 | 2.93 | 1.89 |

PK inhibition activity assessments for compounds herein, as measured by the labeled peptide cleavage assays (Z′LYTE™ assays), fluorescence displacement assay (LanthaScreen™ Eu Kinase Binding Assay) and ADP formation assay (Adapta™ Assay) described in this Example, are presented in FIG. 4A to FIG. 11C as an IC₅₀ value and/or as a percent inhibition value. A value shown without units in FIG. 4A to FIG. 11C is an IC₅₀ value in nanomolar (nM) units. A value shown as a percentage in FIG. 4A to FIG. 11C is percent inhibition value typically determined at a 100 nM concentration of test compound. The header of the first column of each of the tables shown in FIG. 4A to FIG. 11C includes the term “Cmpd,” which designates a test compound assessed by an assay.

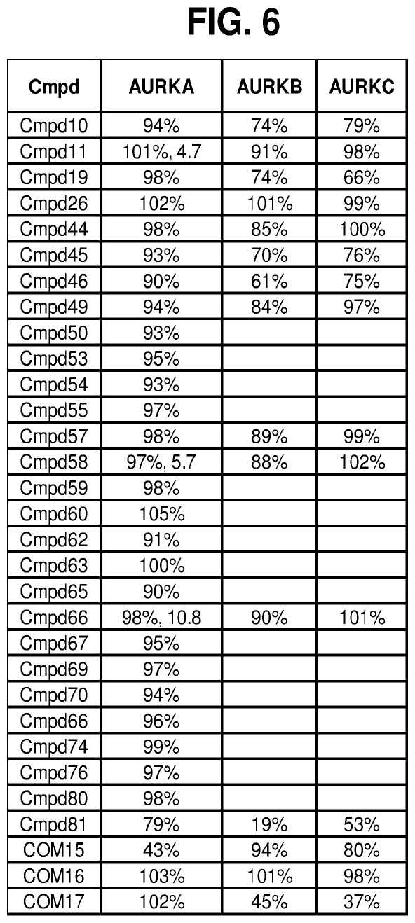

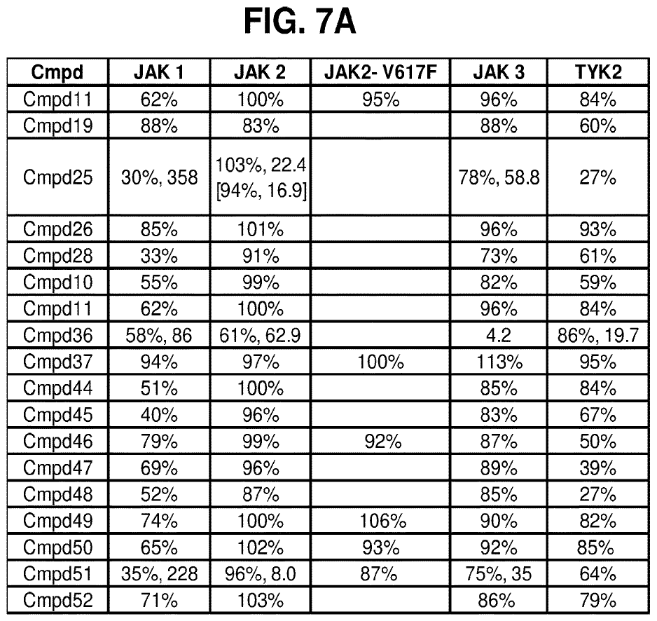

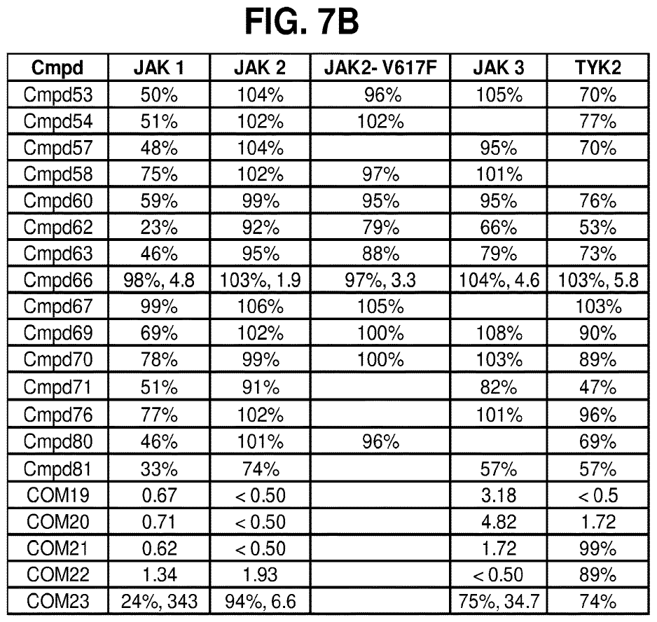

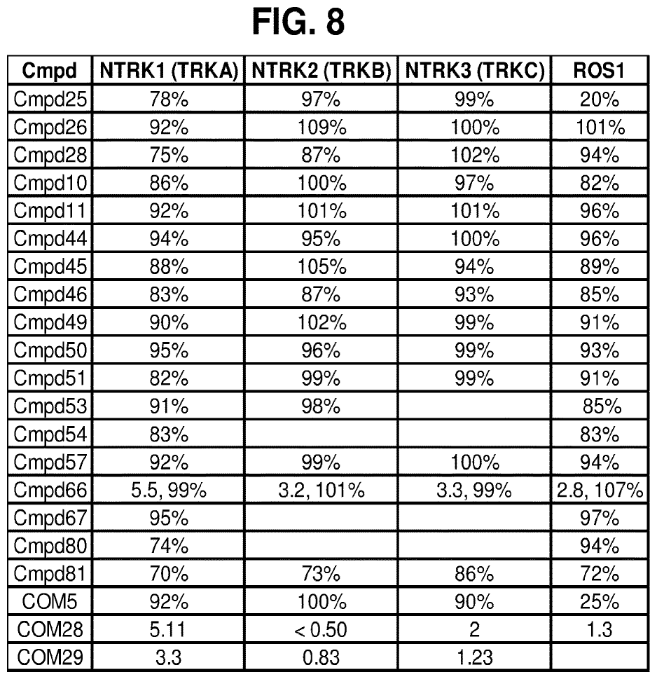

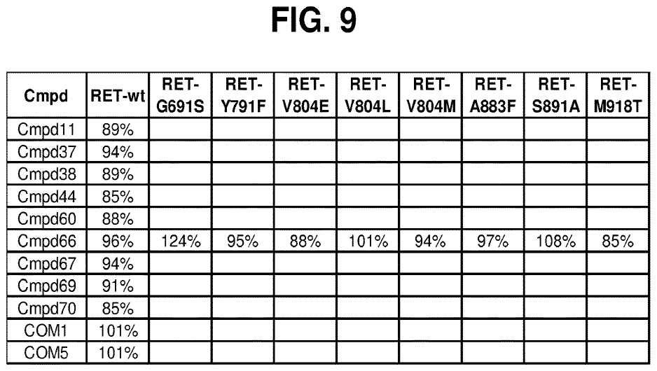

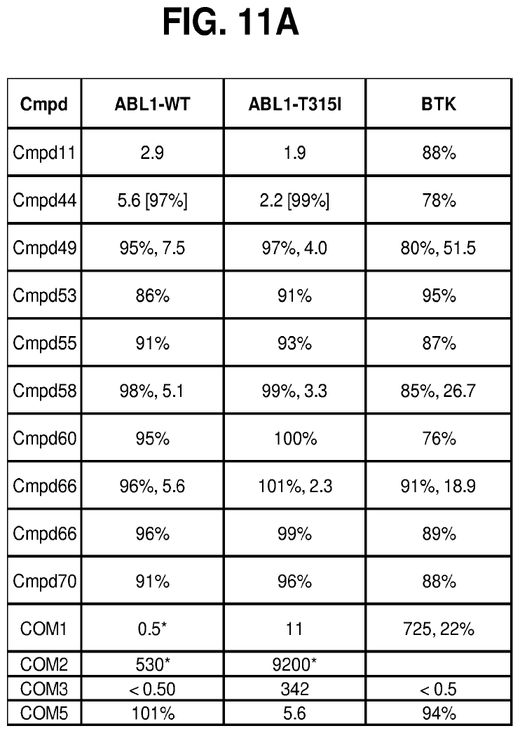

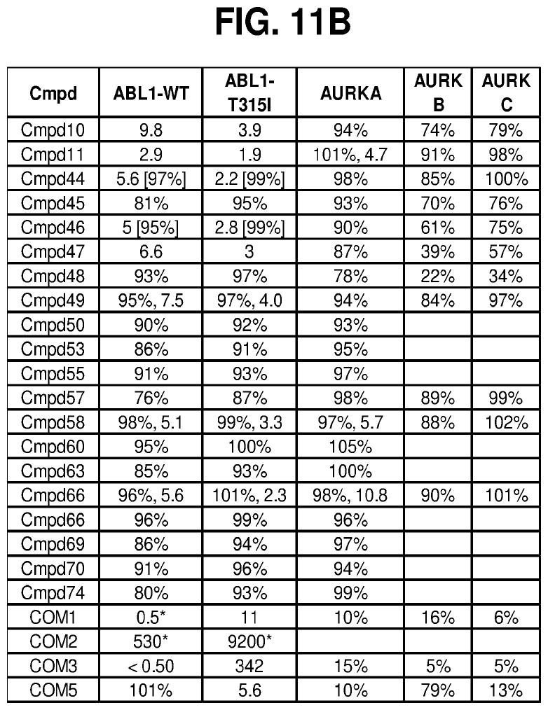

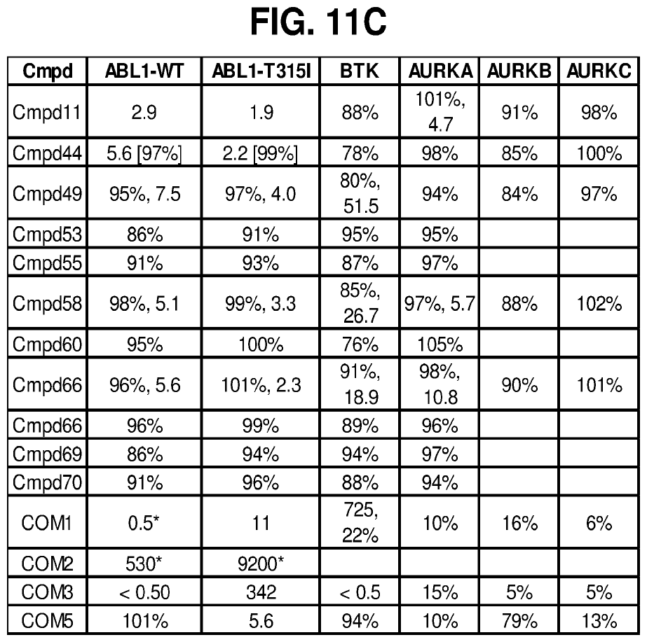

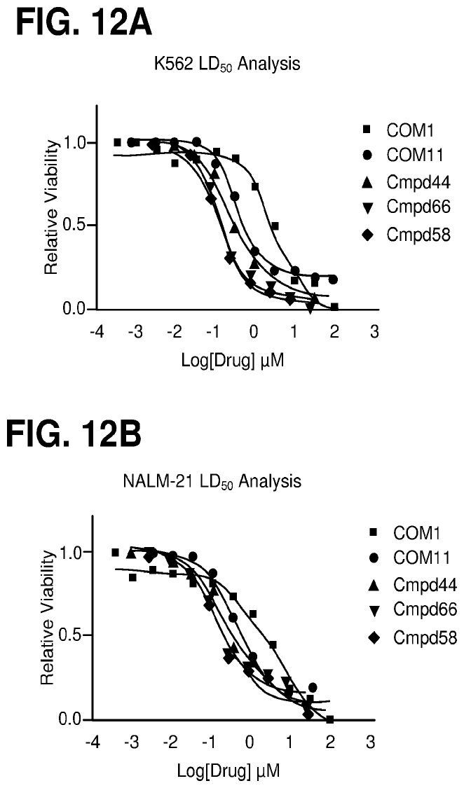

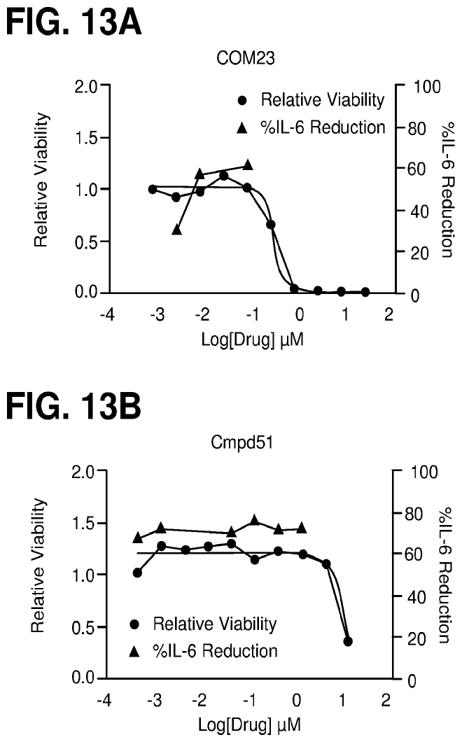

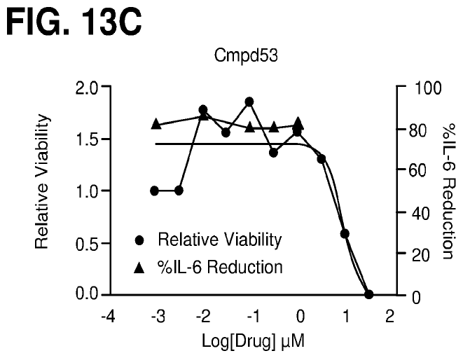

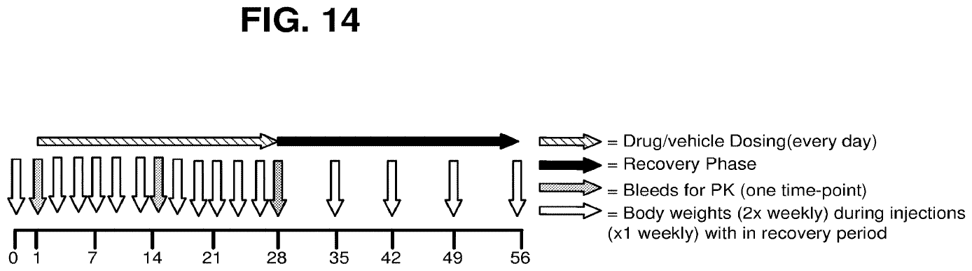

FIG. 4A and FIG. 4B shows inhibition of ABL family PKs. FIG. 5 shows inhibition of BTK family PKs. FIG. 6 shows inhibition of AURK family PKs. FIG. 7 shows inhibition of JAK family PKs. FIG. 8 shows inhibition of TRK family PKs. FIG. 9 shows inhibition of RET family PKs. FIG. 10A shows inhibition of EPH family PKs. FIG. 10B shows inhibition of TNK family, PLK family and IRAK family PKs. FIG. 10C shows inhibition of SRC, LCK and DDR family PKs. FIG. 10D shows inhibition of ABL2 and PTK2B PKs. FIG. 11A shows inhibition of ABL and BTK family PKs. FIG. 11B shows inhibition of ABL and AURK family PKs. FIG. 11C shows inhibition of ABL, BTK and AURK family PKs.

The key at the bottom of FIG. 4B applies to FIG. 4A to FIG. 11C. The top portion of the key is applicable to percent inhibition values and the bottom portion of the key is applicable to IC₅₀ values. In FIG. 4A to FIG. 11C: (i) a “*” designation signifies that the corresponding value is an external value (for example, a value from a scientific publication); (ii) an additional row for a particular test compound within one table signifies a different manufacturing lot of the test compound; (iii) when two percentages are provided in one cell of a table, the first value is obtained at a concentration of 100 nM for the test compound and the second value is obtained at a concentration of 10 nM of the test compound; (iv) a “Lit+ve” designation signifies that the test compound was reported in an external source as exhibiting an inhibitory activity against the applicable target PK (for example, in a scientific publication); and (v) a % value and an IC₅₀ value within one cell of a table for particular test compound are separated by brackets or by a comma (a bracket or comma separator serve the same function of separating the % value and the IC₅₀ value).

In FIG. 4A to FIG. 11C, the “Cmpd” column includes at the end multiple test compounds designated by a “COM” designation, which designates comparative compounds subject to clinical studies that are commercially available. The following “COM” designations are held in reserve: COM4, COM11, COM12, COM13, COM14, COM18, COM24, COM25, COM26 and COM27.

### Example 18: Preparation of Compounds of Formula A1-3

This Example describes processes for preparing compounds of Formula H and Formula J. The following processes prepare compounds of Formula A1-3 in which R¹, R², R³, R¹¹, R¹², R¹³, R¹⁴ R¹⁶ and R¹⁸ each is hydrogen; R⁴, R¹⁵ and R¹⁷ each is methoxy; Y is —N(R^{b})—CH₂—R^{Y}; R^{b} is hydrogen; and R^{Y} is methyl or a polyethylene glycol group. Other compounds of Formula A1-3 can be prepared using similar processes in which R¹, R², R³, R⁴, R¹¹, R¹², R¹³, R¹⁴, R¹⁵, R¹⁶, R¹⁷, R¹⁸, R^{b} and R^{Y} each is substituted by the same or a different substituent.

### Preparation of 2-CI-8-(3,5-Dimethoxyphenyl) Quinazoline by Suzuki Coupling

A 0.5 mL-2.0 mL tapered microwave vial with triangular stir vane is charged with 3,5-dimethoxyphenylboronic acid. A solution of 2-chloro-8-bromo quinazoline in dioxane is added followed by aqueous K₃PO₄. Tetrakis(triphenylphosphine)palladium is added and the vial sealed, then heated in an aluminum block (105° C.) for 5 hours. LCMS indicated the reaction is complete with the formation of the desired product. The reaction is diluted with excess iPrOAc. MgSO₄ is added and the mixture filtered and evaporated to a brown oil (50 mg). The oil is chromatographed by flash chromatography. The major UV-active product is isolated.

### Preparation of 3-((8-(3,5-dimethoxyphenyl)quinazolin-2-yl)amino)-4-methoxybenzaldehyde

A 0.5-2.0 mL tapered microwave vial is charged with 3-amino-4-methoxybenzaldehyde. The vial is then charged with 2-CI-8-(3,5-dimethoxyphenyl)quinazoline. EtOH is added, and the combined solids are agitated using a pipette before sealing. The mixture is placed in a 130° C. aluminum block and vigorously stirred for 5 hours. The vial is rinsed with excess EtOH, then excess DCM, into a 250 mL round-bottomed (rb) flask. The solution is evaporated to an orange-brown oil. IPA (10 mL) is added and the mixture heated until the residue dissolved. The dark solution is allowed to cool and stir at room temperature for 12 hr. The dark brown precipitated solid is recovered by filtration. The product is used without further purification.

### Preparation of 8-(3,5-dimethoxyphenyl)-N-(2-methoxy-5-((methylamino)methyl)phenyl)quinazolin-2-amine

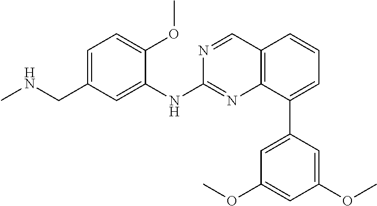

A 20 mL scintillation vial is charged with 3-((8-(3,5-dimethoxyphenyl)quinazolin-2-yl)amino)-4-methoxybenzaldehyde. THF is added to effect dissolution. A solution of methylamine in THF is added followed by solid sodium triacetoxybotohydride. Acetic acid is added and the vial sealed, then stirred at rt for 16 hours.

The reaction is diluted with excess iPrOAc and filtered. The filtrate is washed with 1N aqueous sodium hydroxide (2× washes). The organic phase is washed with aqueous 1N HCl (2× washes). The organic phase is set aside and the aqueous phase is adjusted to pH 12 using solid NaOH. The aqueous phase is extracted with iPrOAc. The aqueous phase is set aside and the organic phase dried over MgSO₄, filtered and evaporated to an oil. The desired product is purified by flash chromatography, yielding the desired compound.

### Preparation of N-(5-(5,8,11-trioxa-2-azadodecyl)-2-methoxyphenyl)-8-(3,5-dimethoxyphenyl)quinazolin-2-amine

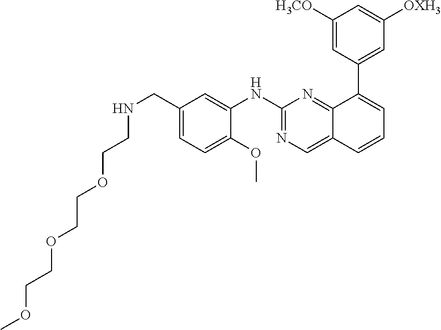

A 20 mL scintillation vial is charged with 3-((8-(3,5-dimethoxyphenyl)quinazolin-2-yl)amino)-4-methoxybenzaldehyde. THF is added to effect dissolution. A solution of 2-(2-(2-methoxyethoxy)ethoxy)ethan-1-amine in THF is added followed by solid sodium triacetoxybotohydride. Acetic acid is added and the vial sealed, then stirred at rt for 16 hours.

The reaction is diluted with excess iPrOAc and filtered. The filtrate is washed with 1N aqueous sodium hydroxide (2× washes). The organic phase is washed with aqueous 1N HCl (2× washes). The organic phase is set aside and the aqueous phase is adjusted to pH 12 using solid NaOH. The aqueous phase is extracted with iPrOAc. The aqueous phase is set aside and the organic phase dried over MgSO₄, filtered and evaporated to an oil. The desired product is purified by flash chromatography, yielding the desired compound.

### Preparation of 8-(3,5-dimethoxyphenyl)-N-(5-(2-(3-((8-(3,5-dimethoxyphenyl)quinazolin-2-yl)amino)-4-methoxybenzyl)-5,8,11-trioxa-2-azadodecyl)-2-methoxyphenyl)quinazolin-2-amine

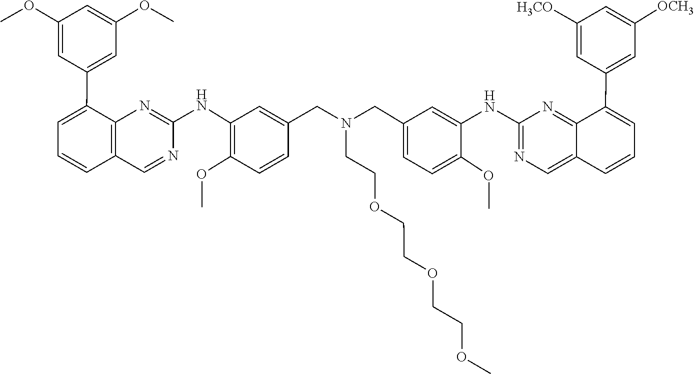 [SMILES: COCCOCCOCCN(Cc1ccc(OC)c(Nc2ncc3cccc(-c4cc(OC)cc(OC)c4)c3n2)c1)Cc1ccc(OC)c(Nc2ncc3cccc(-c4cc(OC)cc(OC)c4)c3n2)c1]

A 20 mL scintillation vial is charged with 3-((8-(3,5-dimethoxyphenyl)quinazolin-2-yl)amino)-4-methoxybenzaldehyde. THF is added to effect dissolution. A solution of N-(5-(5,8,11-trioxa-2-azadodecyl)-2-methoxyphenyl)-8-(3,5-dimethoxyphenyl)quinazolin-2-amine in THF is added followed by solid sodium triacetoxybotohydride. Acetic acid is added and the vial sealed, then stirred at rt for 16 hours.

The reaction is diluted with excess iPrOAc and filtered. The filtrate is washed with 1N aqueous sodium hydroxide (2× washes). The organic phase is washed with aqueous 1N HCl (2× washes). The organic phase is set aside and the aqueous phase is adjusted to pH 12 using solid NaOH. The aqueous phase is extracted with iPrOAc. The aqueous phase is set aside and the organic phase dried over MgSO₄, filtered and evaporated to an oil. The desired product is purified by flash chromatography, yielding the desired compound.

### Preparation of 8-(3,5-dimethoxyphenyl)-N-(5-(((3-((8-(3,5-dimethoxyphenyl)quinazolin-2-yl)amino)-4-methoxybenzyl)(methyl)amino)methyl)-2-methoxyphenyl)quinazolin-2-amine

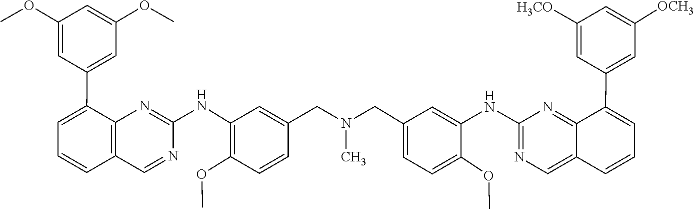 [SMILES: COc1cc(OC)cc(-c2cccc3cnc(Nc4cc(CN(C)Cc5ccc(OC)c(Nc6ncc7cccc(-c8cc(OC)cc(OC)c8)c7n6)c5)ccc4OC)nc23)c1]

A 20 mL scintillation vial is charged with 3-((8-(3,5-dimethoxyphenyl)quinazolin-2-yl)amino)-4-methoxybenzaldehyde. THF is added to effect dissolution. A solution of 8-(3,5-dimethoxyphenyl)-N-(2-methoxy-5-((methylamino)methyl)phenyl)quinazolin-2-amine in THF is added followed by solid sodium triacetoxybotohydride. Acetic acid is added and the vial sealed, then stirred at rt for 16 hours.

The reaction is diluted with excess iPrOAc and filtered. The filtrate is washed with 1N aqueous sodium hydroxide (2× washes). The organic phase is washed with aqueous 1N HCl (2× washes). The organic phase is set aside and the aqueous phase is adjusted to pH 12 using solid NaOH. The aqueous phase is extracted with iPrOAc. The aqueous phase is set aside and the organic phase dried over MgSO₄, filtered and evaporated to an oil. The desired product is purified by flash chromatography, yielding the desired compound.

### Example 19: In Vitro Cytotoxic Activity of BCR-Abl Inhibitors in Cell Lines

Non-metabolized targeted toxicity, or general toxicity, of compounds was assessed by treating a panel of selected cell lines in culture with a dilution series of test compounds and the lethal dose that will kill half of the cells (i.e., the LD₅₀) was determined. Compounds herein were assayed against a panel of cytokine-independent Philadelphia chromosome (BCR-ABL1 translocation) positive (Ph+) leukemia and lymphoma cell lines along with several control cell lines that were ABL1-independent. The panel included murine cell lines BaF3-wildtype (wt), BaF3-mutants (BaF3-wt cells engineered to be reliant on various kinase domain mutations with the BCR-ABL1 fusion protein), and various ALL and CML human cell lines, including SD-1, TOM-1, NALM-21, K562, Jeko-1, BV173, and KCL-22.

To test the cytotoxic activity of compounds herein, 1.6E4 cells were plated in 96-well plates at a final volume of 135 μL per well. Compounds were then added to the wells. In general, final concentrations of compounds started at a maximum concentration of 100 PM and were diluted by half-log concentrations down to a final concentration of 0.316 nM. However, different cell lines required varying drug concentration ranges for optimal LD₅₀ calculations. To create half-log dilutions, 40 mM DMSO solutions of compounds were diluted in half-log iterations to generate 400× starting concentration of compounds in DMSO. The 400× compound solutions were then diluted by 40-fold by adding 5 μL of 400× DMSO stock to 195 μL cell culture media, generating 10× drug concentrations in cell culture media. Then 15 μL of the 10× drug solutions were added to the 135 μL volume of cell suspension, resulting in 1× final drug concentrations with 0.25% DMSO in all wells. 48-hours after adding compounds, cells were measured for relative viability to generate cytotoxicity curves associated with the varying concentrations of compounds. To measure relative viability, CellTiterGlo Luminescent Cell Viability reagent was used, following the manufacturer's suggested protocol (Promega, G7570). LD₅₀ values were measured by normalizing luminescence intensity to the lowest drug concentration. GraphPad Prism was used to plot and calculate LD₅₀ values from the cytotoxic kill curves. Curves were fit to a non-linear regression model, with compound concentration vs. response and variable slope (four parameters).

Table 1 in this Example summarizes the LD₅₀ values calculated from the plots generated as described. In both K562 (CML) and NALM-21 (ALL), compounds referenced in Table 1 of this Example resulted in lower LD₅₀ values, indicating increased levels of cytotoxic potency against the two human tumor cell lines.

| Compound | K562 LD₅₀ (μM) | NALM-21 LD₅₀ (μM) |
|---|---|---|
| ponatinib | 3.25 | 3.60 |
| Cmpd11 | 0.403 | 0.446 |
| Cmpd44 | 0.300 | 0.281 |
| Cmpd66 | 0.141 | 0.133 |
| Cmpd58 | 0.124 | 0.134 |

FIG. 12A and FIG. 12B show representative cytotoxicity curves for ponatinib (control compound) and the compounds listed in Table 1 of this Example against K562 and NALM-21 cells, respectively.

### Example 20: Senomorphic Activity in IMR90 Fibroblast In Vitro Model

Senomorphic activity of compounds were assessed by determining whether the compounds could inhibit the senescence-associated secretory phenotype (SASP) in senescent cells, which is associated with overproduction and secretion of pro-inflammatory factors. Although many of these factors are associated primarily with macrophages and monocytes, they can also be made by a variety of senescent and activated cell types, such as fibroblasts. Specifically, IMR90 fibroblasts are known to overproduce these factors, especially when rendered senescent.

In the assay, IMR90 fibroblasts were plated at a density of 3E6 cells in a T175 tissue culture (TC)-treated flask. The flasks were placed into a tissue culture incubator and cells were allowed to adhere to the bottom of the wells for 24 hours. After cell adherence, the TC-treated flasks containing the cells were placed into a MultiRad225 X-ray irradiator, where cells were exposed to 10 grays (Gy) of ionizing gamma irradiation. After irradiation, flasks were returned to the tissue culture incubator for 7 days, refreshing the cell media every 3 days.

After the 7-day incubation period that allowed full senescence transformation, cells were detached from the TC-treated flasks using 0.25% trypsin-EDTA solution. Cells were then split, and two experiments were conducted simultaneously: (1) verification of senescence induction via β-galactosidase staining (a senescent cell marker) and (2) analysis of senomorphic activity of compounds.

For verification of senescence induction, 2.5E4 cells were plated in TC-treated 6-well plates. After overnight adherence to the TC-plates, cells were fixed and stained for β-galactosidase accumulation following the manufacturer's standard protocol (e.g., Millipore Sigma, Senescence Cells Histochemical Staining Kit, CS0030-1KT). Brightfield microscopy was used to image the cells and ImageJ software was used to calculate the percentage of senescent cells per well. Non-irradiated IM90 fibroblasts were used as a non-senescent control.

To test the senomorphic activity of compounds, 1E4 cells were plated in 96-well plates. After overnight adherence to the 96-well plates, compounds were added in half-log dilutions to the wells containing the cells. Compounds that were effective inhibitors of JAK2 and were not effective inhibitors of JAK1 were assayed (JAK2 selective inhibitors). Twenty-four hours after compounds were added, cell culture media was collected and analyzed by various enzyme linked immunosorbent assays (ELISAs) for the expression of SASP-associated cytokines, including IL-6, TGF-beta, and TNF-alpha. The manufacturer's protocol was followed for ELISA analysis (e.g., Abcam, Human IL-6 ELISA kit, ab178013). Cytokine levels were compared to untreated controls and reduction of cytokine production was plotted and calculated using GraphPad Prism analysis software.

Cells also were measured for relative viability to generate cytotoxicity curves associated with the varying concentrations of compounds. To measure relative viability, CellTiterGlo Luminescent Cell Viability reagent was used, following the manufacturer's suggested protocol (Promega, G7570). LD₅₀ values were measured by normalizing luminescence intensity to the lowest drug concentration. GraphPad Prism was used to plot and calculate LD₅₀ values from the cytotoxic kill curves. Curves were fit to a non-linear regression model, with compound concentration vs. response and variable slope (four parameters).

The relative bioavailability (black circles, left y-axis) and the percentage of IL-6 reduction (black triangles, right y-axis) for the control compound pacritinib, Cmpd51, and Cmpd53 are shown in FIG. 13A, FIG. 13B and FIG. 13C, respectively. Both compounds showed potent IL-6 reduction at sub-toxic drug levels. Cmpd51 reduced IL-6 production by 67.5% at 0.4 nM and 71.6% at 1250 nM, while relative viability remained at a value of 1, indicating reduction of IL-6 without cell toxicity. Cmpd53 reduced IL-6 production by 82.2% at 0.8 nM and 82.9% at 800 nM, while relative bioavailability remained at a value of 1, indicating reduction of IL-6 without cell toxicity. Both Cmpd51 and Cmpd53 were more potent than the control drug pacritinib.

### Example 21: Preparation of 9H-fluoren-9-ylmethyl N-[5-(tert-butoxycarbonylamino)-2-methyl-phenyl]carbamate (Compound A)

The title compound having the following structure was prepared:

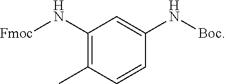

A 250 mL round bottom flask with stir bar was charged with DCM (50 mL). Solid tert-butyl N-(3-amino-4-methyl-phenyl)carbamate (1.98 g, 8.908 mmol) was added and the mixture stirred until dissolution was complete. DIEA (1.86 mL, 10.689 mmol) was added, followed by Fmoc-Cl (2.765 g, 10.689 mmol). The reaction was stirred at room temperature overnight.

The suspension of white solid was filtered and the filtrate evaporated to a white solid. The white solid was suspended in EtOAc (50 mL) and filtered again. The filtrate was evaporated to the title compound (white solid, 3.804 g). The product was taken forward into the next step without further purification.

### Example 22: Preparation of 9H-fluoren-9-ylmethyl N-(5-amino-2-methyl-phenyl)carbamate hydrochloride (Compound B)

The title compound having the following structure was prepared:

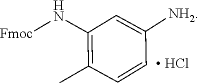

A 250 mL round bottom (rb) flask containing 9H-fluoren-9-ylmethyl N-[5-(tert-butoxycarbonylamino)-2-methyl-phenyl]carbamate was charged with DCM (50 mL). TFA (10 mL) was added and the yellow suspension became homogenous and turned dark brown in 1 hour (hr). The solution was stirred at room temperature overnight.

Toluene (20 mL) was added, and the reaction evaporated to a dark residue (9.205 g). The residue was dissolved in iPrOAc (100 mL) in a 250 mL rb flask with stirrer. 1N HCl (50 mL) was added and the mixture vigorously stirred for 2 hr. A voluminous white solid precipitated. The solid was recovered by filtration from the two-phase mother liquor. The solid in the filter funnel was allowed to stand under lyophilizer vacuum overnight. LCMS showed it is the title compound as the HCl salt (1.132 g).

### Example 23: Preparation of 9H-fluoren-9-ylmethyl N-[5-[(8-bromoquinazolin-2-yl)amino]-2-methyl-phenyl]carbamate (Compound C)

The title compound having the following structure was prepared:

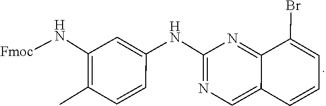

Two 10-20 mL microwave vials with stir bars were each charged with [3-(9H-fluoren-9-ylmethoxycarbonylamino)-4-methyl-phenyl]ammonium; chloride (526 mg, 1.381 mmol). Dioxane (10 mL) was added to each vial and the mixture stirred for 5 min to effect complete suspension. While stirring, each vial was then charged with 8-bromo-2-chloro-quinazoline (673 mg, 2.762 mmol). The vials were sealed and heated in an aluminum block (125° C.) for 1.5 hr. Each reaction becomes a dark orange homogenous solution. The vials were capped with a rubber septum and vigorously stirred at room temperature for 2 hr. The bright orange precipitate was recovered by filtration (filtration was slow), then allowed to stand under lyophilizer vacuum for 3 days, affording the title compound (1.232 g).

### Example 24: Preparation of Cmpd51

The title compound having the following structure was prepared:

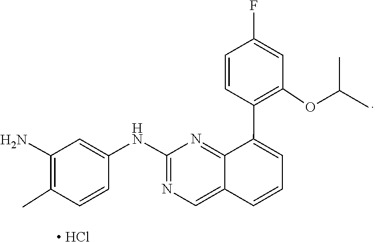 [SMILES: Cc1ccc(Nc2ncc3cccc(-c4ccc(F)cc4OC(C)C)c3n2)cc1N ; "· HCl"]

A 10-20 mL microwave vial was charged with 9H-fluoren-9-ylmethyl N-[5-[(8-bromoquinazolin-2-yl)amino]-2-methyl-phenyl]carbamate (938 mg, 1.701 mmol). Dioxane (12 mL) was added and the mixture stirred for 1 hr at room temperature to effect complete suspension. (4-fluoro-2-isopropoxy-phenyl)boronic acid (505 mg, 2.552 mmol) was added followed by aqueous (aq) K₃PO₄ (2M, 2.6 mL, 5.103 mmol). Solid K₃PO₄ was added (361 mg, 1.701 mmol). Tetrakistriphenylphosphine palladium (393 mg, 0.340 mmol) was added. The vial was sealed and heated in an aluminum block (110 degrees C.) for 16 hr.

The mixture, containing a yellow solid suspended in the two-phase dioxane/water solvent, was poured into iPrOAc (~100 mL). The solid dissolved. The mixture was transferred to a separatory funnel and the lower aqueous was removed and set aside. The clear, brown organic was dried over MgSO₄ and filtered into a 250 mL rb flask. The filtrate was evaporated down to about 50 mL volume, then treated with 1N HCl (50 mL). The orange mixture was vigorously stirred overnight.

The two-phase mixture consisted of a deep yellow aqueous phase and a brown organic phase with a small amount of insoluble residue floating at the interface. The mixture was transferred to a separatory funnel. The slightly turbid aqueous was slowly drawn off to avoid taking any of the insoluble material. The remaining organic was returned to the rb flask and combined with 1N HCl (50 mL). The mixture was vigorously stirred for 1 hr, then carefully partitioned again. The combined aqueous phase was placed into another rb flask and combined with iPrOAc (75 mL), then vigorously stirred for 1 hr. The now clear yellow aqueous was recovered. The aqueous phase was evaporated on a rotary evaporator (rotovap) and the resultant yellow solid was allowed to stand under lyophilizer vacuum overnight, affording the title compound as a yellow/orange solid (490 mg).

### Example 25: Preparation of Cmpd66

The title compound having the following structure was prepared:

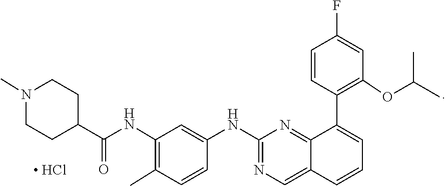 [SMILES: Cc1ccc(Nc2ncc3cccc(-c4ccc(F)cc4OC(C)C)c3n2)cc1NC(=O)C1CCN(C)CC1 ; "· HCl"]

A 2.0-5.0 mL microwave vial was charged with N1-(8-(4-fluoro-2-isopropoxyphenyl)quinazolin-2-yl)-4-methylbenzene-1,3-diamine hydrochloride (see, Cmpd51 in Example 24) (225 mg, 0.513 mmol). DMF (2.5 mL) was added and the mixture stirred until dissolution was complete. 1-Methylpiperidine-4-carboxylic acid (110 mg, 0.769 mmol) was added, followed by DIEA (220 μL, 1.282 mmol) and HATU (292 mg, 0.769 mmol). The vial was capped and stirred at room temperature for 2 hr. LCMS showed the reaction was complete. The reaction was poured into excess iPrOAc and washed with aqueous bicarb (4× washes). The brown organic was dried over MgSO₄, filtered and partially evaporated. The remaining organic (~30 mL) was combined with 1N HCl (30 mL) and vigorously stirred at room temperature for 2 hr. The deep yellow aqueous was recovered and allowed to stand at room temperature overnight. A precipitated yellow solid was recovered by filtration and allowed to stand under lyophilizer vacuum for 24 hr, affording the title compound (77 mg).

### Example 26: Preparation of Cmpd53

The title compound having the following structure was prepared:

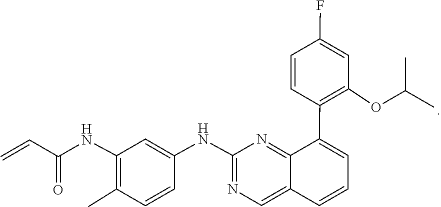 [SMILES: C=CC(=O)Nc1cc(Nc2ncc3cccc(-c4ccc(F)cc4OC(C)C)c3n2)ccc1C]

A 40 mL screw top vial with stir bar was charged with N1-[8-(4-fluoro-2-isopropoxy-phenyl)quinazolin-2-yl]-4-methyl-benzene-1,3-diamine hydrochloride (Cmpd51, Example 24) (209 mg, 0.519 mmol). A solution of acrylic acid (56 mg, 0.779 mmol) in DMF (3 mL) was added, followed by HATU (296 mg, 0.779 mmol). The orange solution was stirred at room temperature overnight.

The reaction mixture was diluted with iPrOAc (~15 mL) and washed with aq bicarb (4× washes). The organic was dried over MgSO₄, filtered and evaporated to a dark brown oil (149 mg). The oil was applied to two 1000 μm pTLC plates and eluted with 20:1 DCM/MeOH, affording the title compound as an orange semi-solid (75 mg).

### Example 27: Preparation of Cmpd21

The title compound having the following structure was prepared:

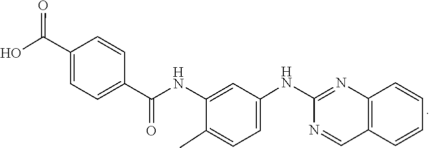 [SMILES: Cc1ccc(Nc2ncc3ccccc3n2)cc1NC(=O)c1ccc(C(=O)O)cc1]

Using the method described above to prepare Compound C (see Example 23), 3-(4-(ethoxycarbonyl)benzamido)benzenaminium 2,2,2-trifluoroacetate was converted to ethyl 4-((2-methyl-5-(quinazolin-2-ylamino)phenyl)carbamoyl)benzoate using 2-chloroquinazoline.

A 10-20 mL microwave vial was charged with ethyl 4-((2-methyl-5-(quinazolin-2-ylamino)phenyl)carbamoyl)benzoate. Tetrahydrofuran (THF) was added and the mixture stirred for 1 min to effect full suspension. LiOH hydrate (10 equivalents) was added, followed by water (10 equivalents). The vial was sealed and heated in an aluminum block (85 degrees C.) for 24 hr.

The solid/liquid mixture was treated with THF and excess 1N HCl. The solid dissolved. The mixture was transferred, using EtOAc, in portions into a screw top vial and evaporated free of organics, leaving a clear aqueous and a suspended solid. The aqueous portion was removed by a syringe. The solid was rinsed twice more with hot water. Each time the water was allowed to cool and was removed by a syringe. The solid was allowed to stand under lyophilizer vacuum overnight, affording the title compound.

### Example 28: Preparation of Cmpd19

The title compound having the following structure was prepared:

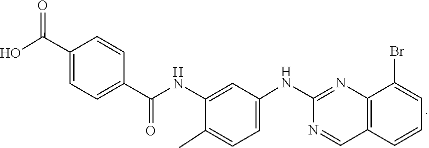 [SMILES: Cc1ccc(Nc2ncc3cccc(Br)c3n2)cc1NC(=O)c1ccc(C(=O)O)cc1]

Using the method described above to prepare Compound C (see Example 23), 3-(4-(ethoxycarbonyl)benzamido)benzenaminium 2,2,2-trifluoroacetate was converted to ethyl 4-((5-((8-bromoquinazolin-2-yl)amino)-2-methylphenyl)carbamoyl)benzoate.

Using the method described above to prepare Cmpd21 (see hydrolysis in Example 27), ethyl 4-((5-((8-bromoquinazolin-2-yl)amino)-2-methylphenyl)carbamoyl)benzoate was converted to the title compound 4-((5-((8-bromoquinazolin-2-yl)amino)-2-methylphenyl)carbamoyl)benzoic acid.

### Example 29: Preparation of Cmpd24

The title compound having the following structure was prepared:

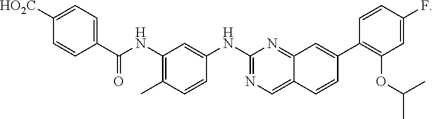 [SMILES: Cc1ccc(Nc2ncc3ccc(-c4ccc(F)cc4OC(C)C)cc3n2)cc1NC(=O)c1ccc(C(=O)O)cc1]

Using the method described above to prepare Compound C (see Example 23), 3-(4-(ethoxycarbonyl)benzamido)benzenaminium 2,2,2-trifluoroacetate was converted to ethyl 4-((5-((7-bromoquinazolin-2-yl)amino)-2-methylphenyl)carbamoyl)benzoate using 2-CI-7-Br-quinazoline.

Using the method described above to prepare Cmpd51 (see Example 24), ethyl 4-((5-((7-bromoquinazolin-2-yl)amino)-2-methylphenyl)carbamoyl)benzoate was converted to ethyl 4-((5-((7-(4-fluoro-2-isopropoxyphenyl)quinazolin-2-yl)amino)-2-methylphenyl)carbamoyl)benzoate.

Using the method described above to prepare Cmpd21 (see hydrolysis in Example 27), ethyl 4-((5-((7-(4-fluoro-2-isopropoxyphenyl)quinazolin-2-yl)amino)-2-methylphenyl)carbamoyl)benzoate was converted to the title compound.

### Example 30: Preparation of Cmpd28

The title compound having the following structure was prepared:

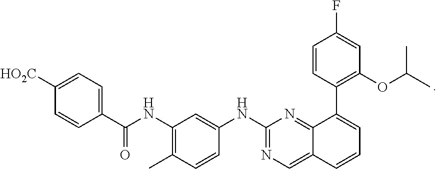 [SMILES: Cc1ccc(Nc2ncc3cccc(-c4ccc(F)cc4OC(C)C)c3n2)cc1NC(=O)c1ccc(C(=O)O)cc1]

A 40 mL screw top vial was charged with 2-Me-5-N-Boc benzoic acid (1.00 g, 3.980 mmol). DMF (12 mL) was added and the yellow mixture stirred at room temperature until dissolution was complete. The vial was then charged with benzocaine (3.29 g, 19.898 mmol) and stirred until homogenous. TEA (1.00 mL, 7.164 mmol) was added followed by EEDQ (4.92 g, 19.898 mmol) with DMF rinsing (total DMF=15 mL). The vial was sealed and stirred at room temperature overnight. LCMS showed the reaction did not go to completion. The reaction was poured into excess iPrOAc and washed with aqueous bicarbonate (3×) followed by 1N HCl (3×). The pale yellow organic phase was dried over MgSO₄, filtered and evaporated to a yellow/orange solid (1.611 g). LCMS showed the acid had been removed but not all the benzocaine. TLC confirmed that benzocaine was present in the crude product. The crude product was loaded onto a 24 gram silica gel column and eluted with a DCM/MeOH gradient (0% to 10% MeOH/DCM over 15 CV, then 20% MeOH/DCM for 7 CV). The fractions were found to be nearly identical from end to end.

Fraction 2 from the 2nd rack was diluted with excess iPrOAc and washed with 1N HCl (3×). LCMS showed the benzocaine in the aqueous phase. The organic phase was washed 6× more with 1N HCl. The organic was dried over MgSO₄, filtered and evaporated to a residue. LCMS showed the benzocaine had been removed and the material was a binary mixture of desired product with an unknown, non-basic impurity with a small amount of the de-boc product aniline.

The residue was dissolved in DCM (5 mL) and treated with TFA (1 mL). The homogenous solution was stirred at room temperature for 24 hr. LCMS showed deprotection was complete. Toluene (5 mL) was added and the solution evaporated to a brown residue. The residue was dissolved in iPrOAc (5 mL) and treated with 1N HCl (10 mL). The mixture was vigorously stirred overnight. LCMS of the two phases showed separation (a minor amount of the product aniline remained in the organic phase). The phases were separated and the organic phase again stirred with 1N HCl for 3 hr. The phases were separated. The combined aqueous phase was basified by addition of solid NaHCO₃. The resultant aqueous phase was extracted with iPrOAc. The organic phase was dried over MgSO₄, filtered and evaporated to a pale yellow residue. LCMS showed the product aniline was present in good purity.

The remaining fractions were recovered and evaporated, then dissolved in iPrOAc (30 mL) and vigorously stirred with 1N HCl (60 mL) for 2 hr. Stirring was stopped and the two layers sampled for LCMS. The benzocaine remaining in the organic was reduced. The procedure was repeated 3 more times (4× in total). The benzocaine was removed.

The organic phase was dried over MgSO₄, filtered and evaporated to an off-white solid. The solid was dissolved in DCM (50 mL) and treated with TFA (10 mL). The clear yellow/orange solution was stirred at room temperature for 1 hr. LCMS showed the reaction was complete. Toluene (20 mL) was added and the reaction evaporated to a residue. The residue was dissolved in iPrOAc (40 mL) and washed once with saturated bicarbonate. The organic phase was dried over MgSO₄, filtered and evaporated to an off white foamy solid. The solid was dissolved in iPrOAc (30 mL) and vigorously stirred with 1N HCl (30 mL) for 2 hr. The mixture was partitioned and the organic sampled. LCMS showed some aniline product still present in the organic so the extraction was repeated. The combined aqueous phase was basified with solid NaHCO₃, extracted with iPrOAc, dried over MgSO₄, filtered and evaporated to give ethyl 4-(5-amino-2-methylbenzamido)benzoate as a light brown foam.

Using the method described above to prepare Compound C (see Example 23), ethyl 4-(5-amino-2-methylbenzamido)benzoate was converted to ethyl 4-(5-((8-bromoquinazolin-2-yl)amino)-2-methylbenzamido)benzoate. Using the method described above to prepare Cmpd51 (see Example 24), 4-(5-((8-bromoquinazolin-2-yl)amino)-2-methylbenzamido)benzoate was converted to ethyl 4-(5-((8-(4-fluoro-2-isopropoxyphenyl)quinazolin-2-yl)amino)-2-methylbenzamido)benzoate. Using the method described above to prepare Cmpd21 (see hydrolysis in Example 27), ethyl 4-(5-((8-(4-fluoro-2-isopropoxyphenyl)quinazolin-2-yl)amino)-2-methylbenzamido)benzoate was converted to the title compound.

### Example 31: Preparation of Cmpd44

The title compound having the following structure was prepared:

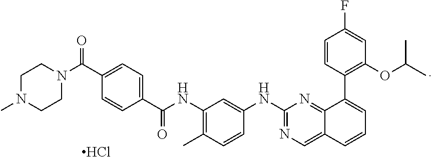 [SMILES: Cc1ccc(Nc2ncc3cccc(-c4ccc(F)cc4OC(C)C)c3n2)cc1NC(=O)c1ccc(C(=O)N2CCN(C)CC2)cc1 ; Cl]

Using the method described above to prepare Cmpd51 (see Example 24), ethyl 4-((5-((8-bromoquinazolin-2-yl)amino)-2-methylphenyl)carbamoyl)benzoate was converted into ethyl 4-((5-((8-(4-fluoro-2-isopropoxyphenyl)quinazolin-2-yl)amino)-2-methylphenyl)carbamoyl)benzoate. Using the method described above to prepare Cmpd21 (see hydrolysis in Example 27), ethyl 4-((5-((8-(4-fluoro-2-isopropoxyphenyl)quinazolin-2-yl)amino)-2-methylphenyl)carbamoyl)benzoate was converted to 4-((5-((8-(4-fluoro-2-isopropoxyphenyl)quinazolin-2-yl)amino)-2-methylphenyl)carbamoyl)benzoic acid.

A 0.5-2.0 mL tapered microwave vial with triangular stir vane was charged with a solution of 4-[[5-[[8-(4-fluoro-2-isopropoxyphenyl)quinazolin-2-yl]amino]-2-methyl-phenyl]carbamoyl]benzoic acid (147 mg, 0.267 mmol) in DMF (2 mL). Neat 1-methylpiperazine (40 mg, 0.400 mmol) was added, followed by DIEA (70 μL, 0.400 mmol). HATU (152 mg, 0.400 mmol) was added, the vial was sealed and stirred at room temperature for 2 hr. LCMS showed the reaction was complete. The dark reaction mixture was poured into excess iPrOAc and washed with aq bicarbonate. The clear yellow organic was dried over MgSO₄, filtered and evaporated to a residue. The residue was redissolved in iPrOAc and treated with 1N HCl (5 mL). The mixture was vigorously stirred for 1 hr, producing a precipitate. All volatiles were removed and the new residue allowed to stand under lyophilizer vacuum overnight. The residue was layered with iPrOAc and vigorously stirred for 4 hr with occasional scraping to loosen adhered solid from the side of the flask. The solid title compound was recovered by filtration.

### Example 32: Preparation of Cmpd45

The title compound having the following structure was prepared:

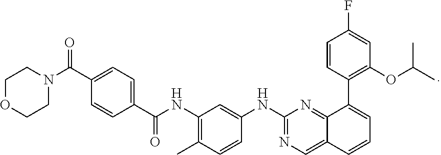 [SMILES: Cc1ccc(Nc2ncc3cccc(-c4ccc(F)cc4OC(C)C)c3n2)cc1NC(=O)c1ccc(C(=O)N2CCOCC2)cc1]

Using the method described above to prepare Cmpd66 (see Example 25), 4-((5-((8-(4-fluoro-2-isopropoxyphenyl)quinazolin-2-yl)amino)-2-methylphenyl)carbamoyl)benzoic acid was converted to the title compound.

### Example 33: Preparation of Cmpd46

The title compound having the following structure was prepared:

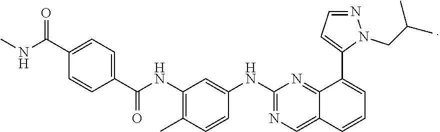 [SMILES: CNC(=O)c1ccc(C(=O)Nc2cc(Nc3ncc4cccc(-c5ccnn5CC(C)C)c4n3)ccc2C)cc1]

A 20 mL scintillation vial was charged with tert-butyl N-(3-amino-4-methyl-phenyl)carbamate (500 mg, 2.20 mmol) followed by 4-(methylcarbamoyl)benzoic acid (725 mg, 4.00 mmol). DMF (7 mL) was added and the mixture stirred until dissolution was complete. TEA (560 μL, 4.00 mmol) was added followed by EEDQ (1.00 g, 4.00 mmol). The initially homogenous yellow solution becomes a finely divided off-white suspension within 2 hours. The mixture was stirred at room temperature overnight. LCMS showed a major product with the anticipated mass. The mixture was diluted with excess iPrOAc, giving white suspension. The suspension was washed with sat aq bicarbonate (3×), then water, then 1N HCl (3×), then water. The mixture remained as a white organic suspension with the aqueous layers separating cleanly, no emulsion. The organic suspension was dried over MgSO₄ and filtered, giving a clear filtrate. The filtrate was evaporated to a tan colored semi-solid (143 mg). The MgSO₄ in the funnel, presumably mixed with the white suspended material, was dissolved in 1N HCl, leaving the white suspended material. The mixture was evaporated free of iPrOAc and filtered. The derived white solid was dried under high vacuum overnight (332 mg), giving tert-butyl N-[4-methyl-3-[[4-(methylcarbamoyl)benzoyl]amino]phenyl]carbamate.

0.5 mL-2.0 mL tapered vial was charged with tert-butyl N-[4-methyl-3-[[4-(methylcarbamoyl)benzoyl]amino]phenyl]carbamate (44 mg, 0.11 mmol) followed by DCM (1.0 mL). The stirred suspension was treated with TFA (0.22 mL). The suspended solid dissolved giving a clear, pale brown solution. The solution was stirred at room temperature for 1 hour. LCMS showed the reaction was complete, giving two peaks of identical mass and isotopic distribution. The rest of the material (288 mg) was subjected to the reaction conditions. The combined product was treated with toluene (8 mL) and evaporated to a dark oil. The oil was rinsed into a separatory funnel containing excess EtOAc and aq bicarb, causing the formation of a voluminous off-white precipitate which obscured the phase separation. The two-phase mixture was filtered with rinsing using excess EtOAc. The derived off-white solid was pull-dried for 2 hours until it became granular and could be removed from the filter funnel with a spatula. The solid was digested in excess IPA at reflux, causing most of it to dissolve and leaving behind some insoluble material, presumably inorganic salts. The IPA solution was allowed to cool to room temperature and filtered. The filtrate was evaporated to a white solid (189 mg). LCMS showed two widely separated peaks having identical masses including the isotopic distribution that conforms to theoretical (M+H) for the desired product N1-(5-amino-2-methyl-phenyl)-N4-methyl-terephthalamide.

Two 0.5-2.0 mL tapered vials with triangular stirring vanes were equally charged with N1-(5-amino-2-methyl-phenyl)-N4-methyl-terephthalamide (147 mg total, 0.519 mmol). EtOH (3.0 mL total) was added followed by 8-bromo-2-chloro-quinazoline (632 mg total, 2.594 mmol). The vials were sealed and heated in an aluminum block (125°) for 4 hours. LCMS showed the two reactions were complete. The reaction mixtures were combined and evaporated to a yellow solid. The solid was digested in refluxing IPA (40 mL), then allowed to cool to room temperature and stir for 4 hr. The yellow solid was recovered by filtration (254 mg) affording the desired N1-[5-[(8-bromoquinazolin-2-yl)amino]-2-methyl-phenyl]-N4-methyl-terephthalamide.

Two 0.5-2.0 mL tapered microwave vials were each charged with N1-[5-[(8-bromoquinazolin-2-yl)amino]-2-methyl-phenyl]-N4-methyl-terephthalamide (127 mg each, 254 mg total, 0.518 mmol). Dioxane (1 mL each) was added followed by (2-isobutylpyrazol-3-yl)boronic acid (66 mg each, 131 mg total, 0.777 mmol) as a turbid solution in dioxane (1 mL each). Aqueous K₃PO₄ (2M, 0.39 mL each, 1.554 mmol total) was added. Tetrakis-TPP-palladium (60 mg each, 120 mg total, 0.104 mmol) was added, the vials were sealed and heated in an aluminum block (105° C.) for 5 hours. The dark reaction mixture was diluted with excess iPrOAc and dried over MgSO₄. The mixture was filtered and evaporated to an orange semisolid (368 mg). The crude product was digested in IPA (9 mL) at reflux. Some light yellow solid did not dissolve. The light yellow solid with red supernatant was stirred at room temperature for 4 hr, then recovered by filtration (134 mg).

A 25 mg portion of the solid was placed in a 4 mL screw to vial. n-Propanol (2 mL) was added and the stirred mixture was brought to near reflux, causing the solid to dissolve. The vial was sealed and stirred at room temperature overnight. The vial was clamped at an angle such that the precipitated solid could settle into a corner of the vial beneath the clear supernatant. LCMS showed the supernatant was a binary mixture with removal of most of the most retained impurity. The supernatant was removed and the procedure was repeated with the mixture stirred at room temperature for 4 hr. LCMS of the second extract showed a similar profile to the first extract except that the desired product was present in lower proportion. The procedure was repeated a third time. The combined extracts were evaporated to a yellow solid. TLC (20:1 DCM/MeOH, 3× elution) showed three major spots with some faint ones. The material was applied to a 500 μm pTLC plate and eluted (3×) with 20:1 DCM/MeOH. The less polar major UV active band was collected, affording the title compound.

### Example 34: Preparation of Cmpd52

The title compound having the following structure was prepared:

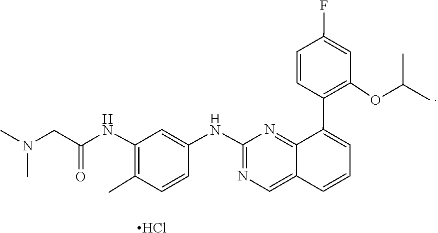 [SMILES: Cc1ccc(Nc2ncc3cccc(-c4ccc(F)cc4OC(C)C)c3n2)cc1NC(=O)CN(C)C ; Cl]

A 0.5-2.0 mL tapered microwave vial with triangular stir vane was charged with [5-[[8-(4-fluoro-2-isopropoxy-phenyl)quinazolin-2-yl]amino]-2-methyl-phenyl]ammonium;chloride (55 mg, 0.125 mmol). DMF (1.0 mL) was added and the mixture stirred for 5 min to effect full suspension of the solid. DIEA (55 μL, 0.313 mmol) was added. 2-(dimethylamino)acetic acid (19 mg, 0.188 mmol) was added, followed by HATU (71 mg, 0.188 mmol). The yellow mixture was stirred at room temperature for 36 hr. LCMS showed the reaction was complete. The reaction mixture was diluted with excess iPrOAc and washed with aqueous bicarb (4× washes), then dried over MgSO₄, filtered and evaporated to a crude residue (50 mg). The residue was dissolved in iPrOAc (4 mL) and combined with 1N HCl (2 mL). The two-phase mixture was stirred at room temperature for 1 hour. Stirring was stopped and the clear, yellow aqueous was drawn off by syringe. The operation was repeated twice, the combined aqueous extractions were evaporated, then lyophilized. The derived salt (orange solid) was difficult to scrape out of the vial. 2.6 mg were provided for biological assays. The remaining material was dissolved in MeOH and transferred to a 20 mL vial, evaporated and allowed to stand under lyophilizer vacuum for 2 hr (32 mg), yielding the title compound.

### Example 35: Preparation of Cmpd55

The title compound having the following structure was prepared:

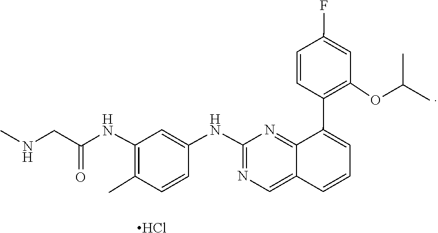 [SMILES: CNCC(=O)Nc1cc(Nc2ncc3cccc(-c4ccc(F)cc4OC(C)C)c3n2)ccc1C ; Cl]

A 0.5-2.0 mL tapered microwave vial with triangular stir vane was charged with [5-[[8-(4-fluoro-2-isopropoxy-phenyl)quinazolin-2-yl]amino]-2-methyl-phenyl]ammonium;chloride (50 mg, 0.114 mmol). DMF (1.0 mL) was added and the mixture stirred for 5 min to effect full suspension. 2-[tert-butoxycarbonyl(methyl)amino]acetic acid (32 mg, 0.171 mmol) was added, followed by DIEA (50 μL, 0.285 mmol). HATU (65 mg, 0.171 mmol) was added and the yellow/orange mixture stirred at room temperature for 4 hr. LCMS showed the reaction was complete. The reaction was partitioned between excess iPrOAc and aqueous bicarbonate. The organic was washed with bicarbonate (4× washes total), dried over MgSO₄, filtered and evaporated to a clear brown residue. The residue was advanced to the de-Boc step without quantification.

A 125 mL rb flask containing the crude product above was treated with DCM (5 mL). TFA (1 mL) was added and the solution stirred at room temperature overnight. LCMS showed the reaction was complete. The reaction was treated with toluene (10 mL) and evaporated to a clear brown residue. The residue was dissolved in iPrOAc (7 mL) and treated with 1N HCl (3 mL). The 2-phase mixture was stirred for 1 hour, giving a flocculent precipitate. The mixture was evaporated free of all volatiles, leaving a yellow semi-solid residue. The residue triturated with iPrOAc (~8 mL). Scraping produced a mixture of filterable solid and semi-solid residue. Acetone (~1 mL) was added and the mixture left to stir over a weekend. The now granular yellow solid was recovered by filtration and allowed to stand under lyophilizer vacuum for 3 hr (29 mg) affording the title compound.

### Example 36: Preparation of Cmpd58

The title compound having the following structure was prepared:

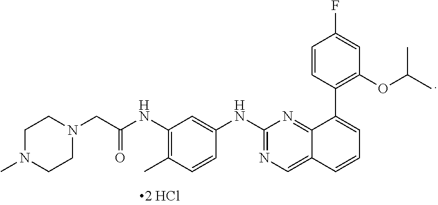 [SMILES: Cc1ccc(Nc2ncc3cccc(-c4ccc(F)cc4OC(C)C)c3n2)cc1NC(=O)CN1CCN(C)CC1]

Using the method for Cmpd66 (see Example 25), N1-(8-(4-fluoro-2-isopropoxyphenyl)quinazolin-2-yl)-4-methylbenzene-1,3-diamine hydrochloride was converted, using 2-(4-methylpiperazin-1-yl)acetic acid, into the title compound.

### Example 37: Preparation of Cmpd29

A Suzuki coupling/hydrolysis process was performed:

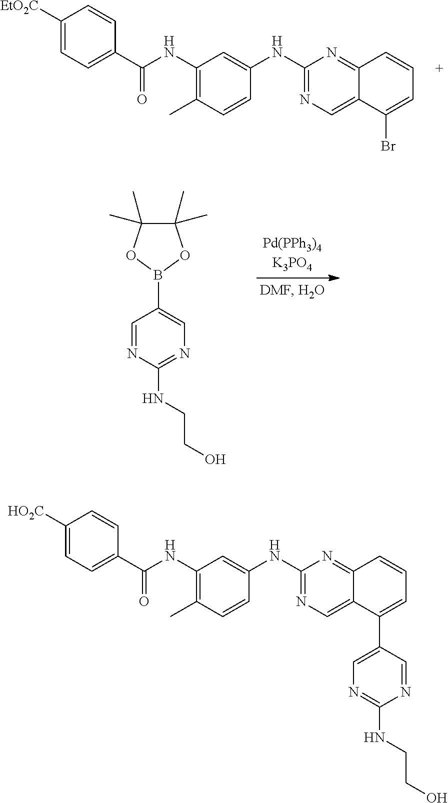 [SMILES: CCOC(=O)c1ccc(C(=O)Nc2cc(Nc3ncc4c(Br)cccc4n3)ccc2C)cc1 ; CC1(C)OB(c2cnc(NCCO)nc2)OC1(C)C ; Cc1ccc(Nc2ncc3c(-c4cnc(NCCO)nc4)cccc3n2)cc1NC(=O)c1ccc(C(=O)O)cc1]

A 0.5 mL-2.0 mL tapered microwave vial with triangular stir vane was charged with ethyl 4-((5-((5-bromoquinazolin-2-yl)amino)-2-methylphenyl)carbamoyl)benzoate (100 mg, 0.198 mmol, 1 equiv), 2-((5-(4,4,5,5-tetramethyl-1,3,2-dioxaborolan-2-yl)pyrimidin-2-yl)amino)ethan-1-ol (68.2 mg, 0.257 mmol, 1.3 equiv), potassium phosphate (594 μL, 1.19 mmol, 3 equiv), and DMF (1.98 mL, 0.1 M). Tetrakis(triphenylphosphine)palladium(0) (45.7 mg, 0.04 mmol, 0.2 equiv) was added, and the vial sealed, then heated in an aluminum block (105° C.) for 1 hour. The reaction was diluted with 1N HCl to ensure the acid was in the organic layer. The layers were separated, and the acid layer washed with ethyl acetate (×2). The combined organic layers were then washed with water (×2) to remove any remaining DMF. The organic layer was then treated with 1M NaOH solution (3×) to separate the carboxylate from any impurities in the organic layer.

After the NaOH-treated phase was confirmed by LCMS to contain only product, the solution was treated with 6M HCl, by dropwise addition of acid, to neutralize any excess hydroxide. Acid product precipitated. The product was then filtered, and the solid material was isolated after lyophilization overnight (12.3 mg isolated, 11.6% yield).

### Example 38: Preparation of Cmpd30

A Suzuki coupling process was performed:

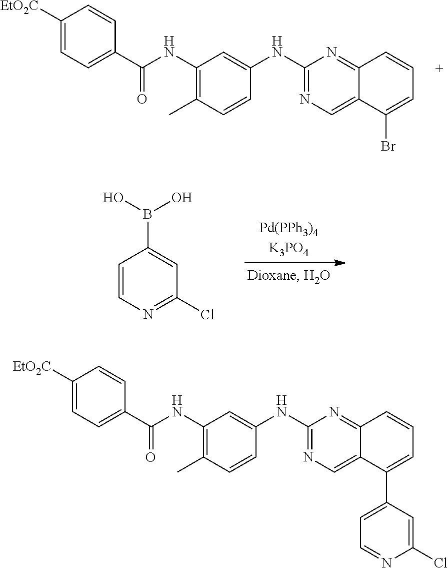 [SMILES: CCOC(=O)c1ccc(C(=O)Nc2cc(Nc3ncc4c(Br)cccc4n3)ccc2C)cc1 ; OB(O)c1ccnc(Cl)c1 ; CCOC(=O)c1ccc(C(=O)Nc2cc(Nc3ncc4c(-c5ccnc(Cl)c5)cccc4n3)ccc2C)cc1]

A 0.5 mL-2.0 mL tapered microwave vial with triangular stir vane was charged with ethyl 4-((5-((5-bromoquinazolin-2-yl)amino)-2-methylphenyl)carbamoyl)benzoate (100 mg, 0.198 mmol, 1 equiv), (2-chloropyridin-4-yl)boronic acid (40.5 mg, 0.257 mmol, 1.3 equiv), potassium phosphate (594 μL, 1.19 mmol, 6 equiv), and dioxane (1.98 mL, 0.1 M). Tetrakis(triphenylphosphine)palladium(0) (45.7 mg, 0.04 mmol, 0.2 equiv) was added, and the vial sealed, then heated in an aluminum block (105° C.) for 12 hours. The reaction was diluted with excess iPrOAc, dried over MgSO₄, and concentrated under reduced pressure. The crude material was purified by pTLC (40:1 CH₂Cl₂:MeOH). One band was isolated containing pure product (71.8 mg, 68% yield).

An ester hydrolysis then was performed:

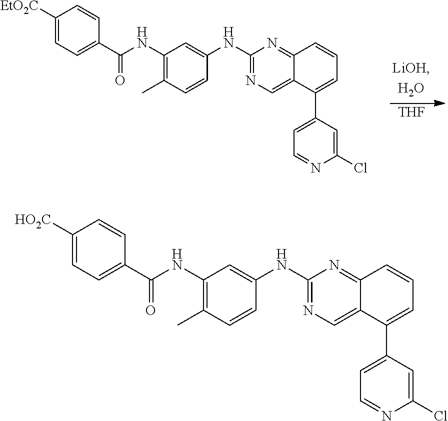 [SMILES: CCOC(=O)c1ccc(C(=O)Nc2cc(Nc3ncc4c(-c5ccnc(Cl)c5)cccc4n3)ccc2C)cc1 ; Cc1ccc(Nc2ncc3c(-c4ccnc(Cl)c4)cccc3n2)cc1NC(=O)c1ccc(C(=O)O)cc1 ; "THF"]

A 0.5-2.0 mL tapered microwave vial with triangular stir vane was charged with a suspension of ethyl 4-((5-((5-(2-chloropyridin-4-yl)quinazolin-2-yl)amino)-2-methylphenyl)carbamoyl)benzoate (71.8 mg, 0.134 mmol) in THF (2.6 mL). Water (0.69 mL, 1.34 mmol) was added followed by LiOH hydrate (56.1 mg, 1.34 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight. Pure product was recovered (6.8 mg, 10% yield).

### Example 39: Preparation of Cmpd31

A Suzuki coupling process was performed:

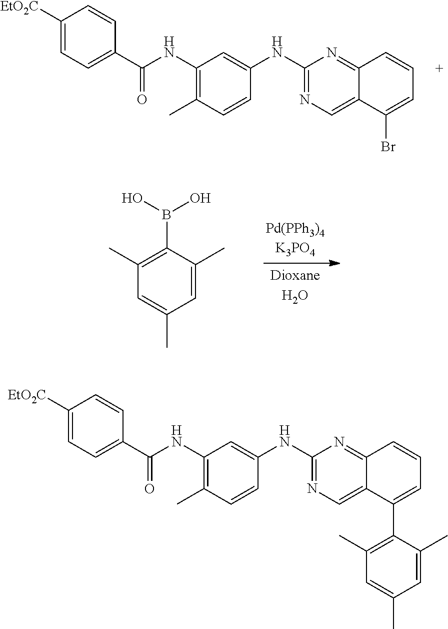

A 0.5 mL-2.0 mL tapered microwave vial with triangular stir vane was charged with ethyl 4-((5-((5-bromoquinazolin-2-yl)amino)-2-methylphenyl)carbamoyl)benzoate (100 mg, 0.198 mmol, 1 equiv), mesitylboronic acid (42.2 mg, 0.257 mmol, 1.3 equiv), potassium phosphate (594 μL, 1.19 mmol, 3 equiv), and dioxane (1.98 mL, 0.1 M). Tetrakis(triphenylphosphine)palladium(0) (45.7 mg, 0.04 mmol, 0.2 equiv) was added, and the vial sealed, then heated in an aluminum block (105° C.) for 12 hour. The reaction was diluted with excess iPrOAc, dried over MgSO₄, and concentrated under reduced pressure. The crude material was purified by pTLC (40:1 CH₂Cl₂:MeOH). One band was isolated containing pure product (28.4 mg, 26% yield).

An ester hydrolysis then was performed:

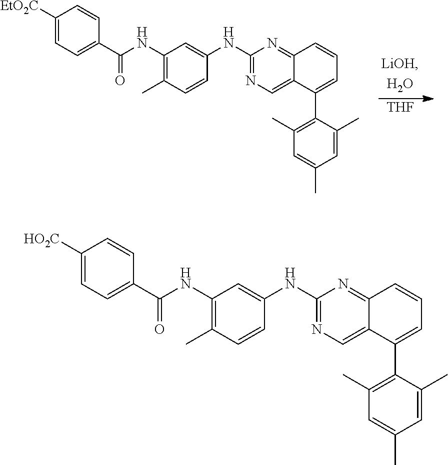 [SMILES: CCOC(=O)c1ccc(C(=O)Nc2cc(Nc3ncc4c(-c5c(C)cc(C)cc5C)cccc4n3)ccc2C)cc1 ; Cc1cc(C)c(-c2cccc3nc(Nc4ccc(C)c(NC(=O)c5ccc(C(=O)O)cc5)c4)ncc23)c(C)c1 ; "THF"]

A 0.5-2.0 mL tapered microwave vial with triangular stir vane was charged with a suspension of ethyl 4-((5-((5-mesitylquinazolin-2-yl)amino)-2-methylphenyl)carbamoyl)benzoate (28.4 mg, 0.05 mmol) in THF (0.52 mL). Water (0.26 mL, 0.52 mmol) was added followed by LiOH hydrate (22 mg, 0.52 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight. Pure product was recovered (14 mg, 52% yield).

### Example 40: Preparation of Cmpd32

A Suzuki coupling process was performed:

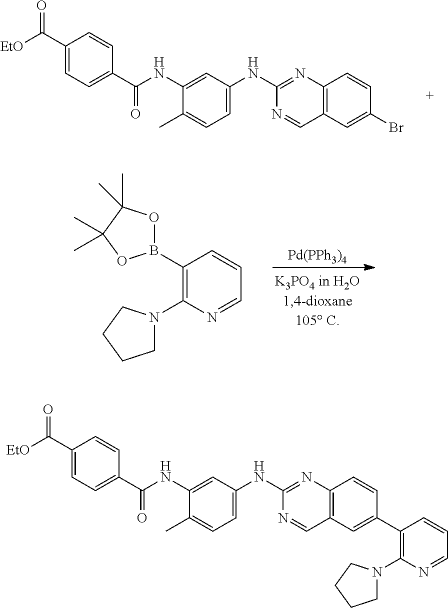 [SMILES: CCOC(=O)c1ccc(C(=O)Nc2cc(Nc3ncc4cc(Br)ccc4n3)ccc2C)cc1 ; CC1(C)OB(c2cccnc2N2CCCC2)OC1(C)C ; CCOC(=O)c1ccc(C(=O)Nc2cc(Nc3ncc4cc(-c5cccnc5N5CCCC5)ccc4n3)ccc2C)cc1 ; "Pd(PPh3)4"]

A 0.5 mL-2.0 mL tapered microwave vial with triangular stir vane was charged with ethyl 4-((5-((6-bromoquinazolin-2-yl)amino)-2-methylphenyl)carbamoyl)benzoate (40 mg, 0.08 mmol, 1 equiv), 2-(pyrrolidin-1-yl)-3-(4,4,5,5-tetramethyl-1,3,2-dioxaborolan-2-yl)pyridine (32 mg, 0.12 mmol, 1.5 equiv), potassium phosphate (119 μL, 0.237 mmol, 3 equiv), and dioxane (0.44 mL, 0.18 M). Tetrakis(triphenylphosphine)palladium(0) (50.4 mg, 0.016 mmol, 0.2 equiv) was added, and the vial sealed, then heated in an aluminum block (105° C.) overnight. The reaction was diluted with excess iPrOAc and dried over MgSO₄. Filtration and evaporation afforded a yellow solid. The crude material was purified by pTLC (40:1 CH₂Cl₂:MeOH). One band was isolated containing product, Rf: 0.7. The material was taken on to the next step in slightly impure form.

An ester hydrolysis then was performed:

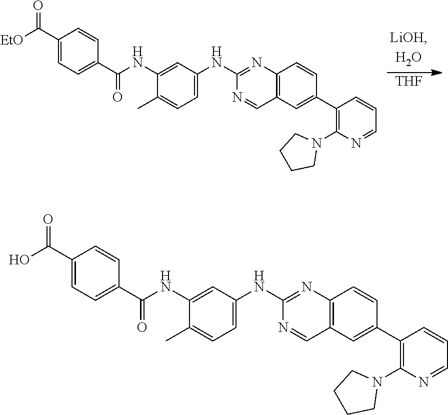 [SMILES: CCOC(=O)c1ccc(C(=O)Nc2cc(Nc3ncc4cc(-c5cccnc5N5CCCC5)ccc4n3)ccc2C)cc1 ; Cc1ccc(Nc2ncc3cc(-c4cccnc4N4CCCC4)ccc3n2)cc1NC(=O)c1ccc(C(=O)O)cc1 ; "THF"]

A 0.5-2.0 mL tapered microwave vial with triangular stir vane was charged with a suspension of ethyl 4-((2-methyl-5-((6-(2-(pyrrolidin-1-yl)pyridin-3-yl)quinazolin-2-yl)amino)phenyl)carbamoyl)benzoate (45.3 mg, 0.08 mmol) in THF (3.96 mL). Water (0.4 mL, 0.8 mmol) was added followed by LiOH hydrate (33.2 mg, 0.8 mmol). The vial was sealed and heated in an aluminum block (85° C.) for 12 hr. The reaction was diluted with 1M HCl. Solid precipitated and the HCl was removed. The material was then washed with water (3×). Water was removed via syringe and the material was lyophilized overnight. Pure product was recovered (6.2 mg, 14% yield).

### Example 41: Reserved

### Example 42: Preparation of Cmpd36

A Suzuki coupling process was performed:

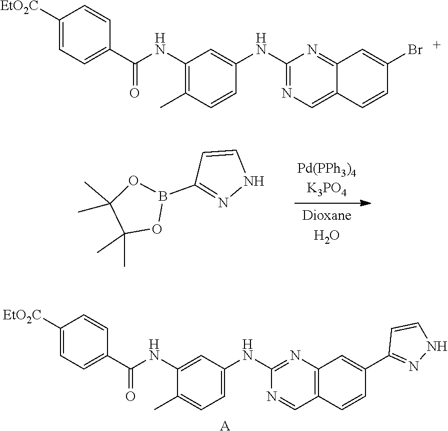

0.5 mL-2.0 mL tapered microwave vial with triangular stir vane was charged with ethyl 4-((5-((7-bromoquinazolin-2-yl)amino)-2-methylphenyl)carbamoyl)benzoate (100 mg, 0.198 mmol, 1 equiv), 3-(4,4,5,5-tetramethyl-1,3,2-dioxaborolan-2-yl)-1H-pyrazole (58 mg, 0.297 mmol, 1.5 equiv), potassium phosphate (297 μL, 0.594 mmol, 3 equiv), and dioxane (1.1 mL, 0.18 M). Tetrakis(triphenylphosphine)palladium(0) (45.7 mg, 0.04 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂Cl₂:MeOH). One band was isolated containing pure product (18.3 mg, 19% yield).

An ester hydrolysis then was performed:

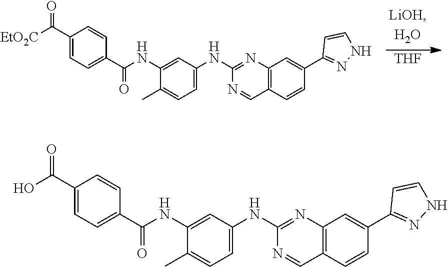 [SMILES: CCOC(=O)C(=O)c1ccc(C(=O)Nc2cc(Nc3ncc4ccc(-c5cc[nH]n5)cc4n3)ccc2C)cc1 ; Cc1ccc(Nc2ncc3ccc(-c4cc[nH]n4)cc3n2)cc1NC(=O)c1ccc(C(=O)O)cc1 ; "THF"]

A 0.5-2.0 mL tapered microwave vial with triangular stir vane was charged with a suspension of ethyl 4-((5-(7-(10H-pyrazol-3-yl)quinazolin-2-yl)amino)-2-methylphenyl)carbamoylbenzoate (18.3 mg, 0.37 mmol) in THF (0.66 mL). Water (0.186 mL, 0.37 mmol) was added followed by LiOH hydrate (15.6 mg, 0.37 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight to afford pure product (9.3 mg, 54% yield).

### Example 43: Preparation of Cmpd37

A Suzuki coupling process was performed:

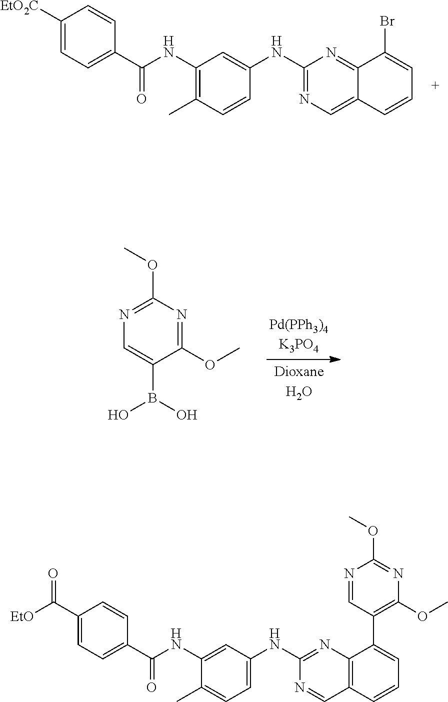 [SMILES: CCOC(=O)c1ccc(C(=O)Nc2cc(Nc3ncc4cccc(Br)c4n3)ccc2C)cc1 ; COc1ncc(B(O)O)c(OC)n1 ; CCOC(=O)c1ccc(C(=O)Nc2cc(Nc3ncc4cccc(-c5cnc(OC)nc5OC)c4n3)ccc2C)cc1]

A 0.5 mL-2.0 mL tapered microwave vial with triangular stir vane was charged with ethyl 4-((5-((8-bromoquinazolin-2-yl)amino)-2-methylphenyl)carbamoyl)benzoate (100 mg, 0.198 mmol, 1 equiv), 2,4-Dimethoxypyrimidine-5-boronic acid (55 mg, 0.297 mmol, 1.5 equiv), potassium phosphate (297 μL, 0.594 mmol, 3 equiv), and dioxane (1.1 mL, 0.18 M). Tetrakis(triphenylphosphine)palladium(0) (45.7 mg, 0.04 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂Cl₂:MeOH). One band was isolated containing product.

An ester hydrolysis then was performed:

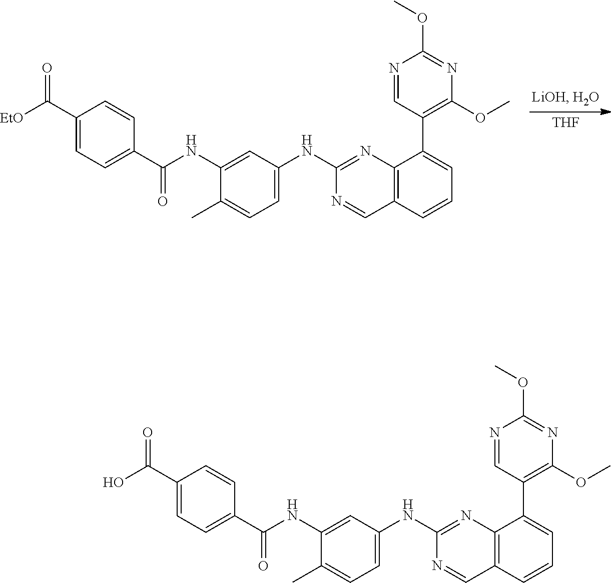

A 0.5-2.0 mL tapered microwave vial with triangular stir vane was charged with a suspension of ethyl 4-((5-((8-(2,4-dimethoxypyrimidin-5-yl)quinazolin-2-yl)amino)-2-methylphenyl)carbamoyl)benzoate (137 mg, 0.243 mmol) in THF (4.33 mL). Water (1.213 mL, 2.43 mmol) was added followed by LiOH hydrate (102 mg, 2.43 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight to afford pure product (13.7 mg, 11% yield).

### Example 44: Preparation of Cmpd38

A Suzuki coupling process was performed:

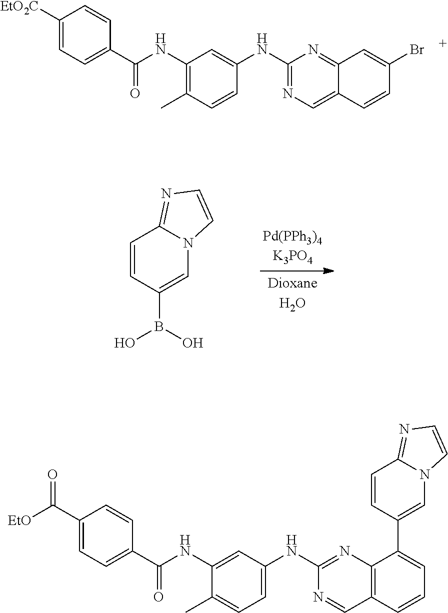 [SMILES: CCOC(=O)c1ccc(C(=O)Nc2cc(Nc3ncc4ccc(Br)cc4n3)ccc2C)cc1 ; OB(O)c1ccc2nccn2c1 ; CCOC(=O)c1ccc(C(=O)Nc2cc(Nc3ncc4cccc(-c5ccc6nccn6c5)c4n3)ccc2C)cc1]

A 0.5 mL-2.0 mL tapered microwave vial with triangular stir vane was charged with ethyl 4-((5-((8-bromoquinazolin-2-yl)amino)-2-methylphenyl)carbamoyl)benzoate (100 mg, 0.198 mmol, 1 equiv), Imidazo[1,2-a]pyridine-6-boronic acid (48 mg, 0.297 mmol, 1.5 equiv), potassium phosphate (297 μL, 0.594 mmol, 3 equiv), and dioxane (1.1 mL, 0.18 M). Tetrakis(triphenylphosphine)palladium(0) (45.7 mg, 0.04 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂Cl₂:MeOH). One band was isolated containing pure product (13.7 mg, 13% yield).

An ester hydrolysis then was performed:

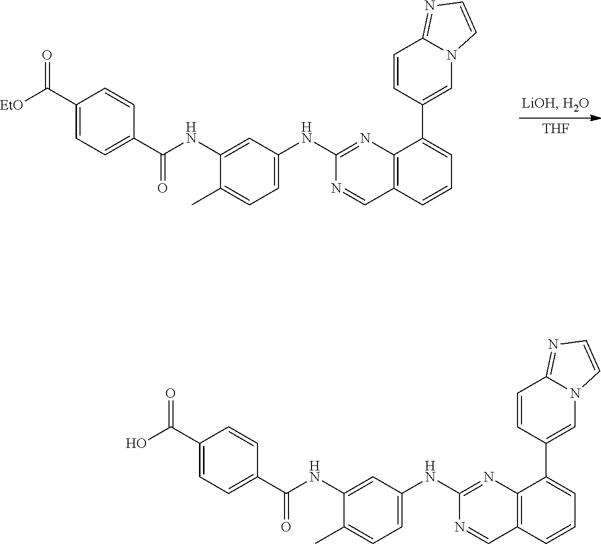 [SMILES: CCOC(=O)c1ccc(C(=O)Nc2cc(Nc3ncc4cccc(-c5ccc6nccn6c5)c4n3)ccc2C)cc1 ; Cc1ccc(Nc2ncc3cccc(-c4ccc5nccn5c4)c3n2)cc1NC(=O)c1ccc(C(=O)O)cc1 ; "THF"]

A 0.5-2.0 mL tapered microwave vial with triangular stir vane was charged with a suspension of ethyl 4-((5-((8-(imidazo[1,2-a]pyridin-6-yl)quinazolin-2-yl)amino)-2-methylphenyl)carbamoyl)benzoate (13.7 mg, 0.025 mmol) in THF (0.5 mL). Water (0.126 mL, 0.25 mmol) was added followed by LiOH hydrate (10.6 mg, 0.25 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight to afford pure product (5.3 mg, 41% yield).

### Example 45: Preparation of Cmpd40

The title compound having the following structure was prepared:

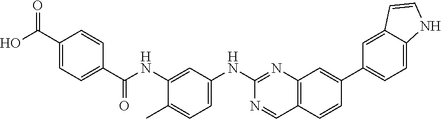 [SMILES: Cc1ccc(Nc2ncc3ccc(-c4ccc5[nH]ccc5c4)cc3n2)cc1NC(=O)c1ccc(C(=O)O)cc1]

A Suzuki coupling process was performed:

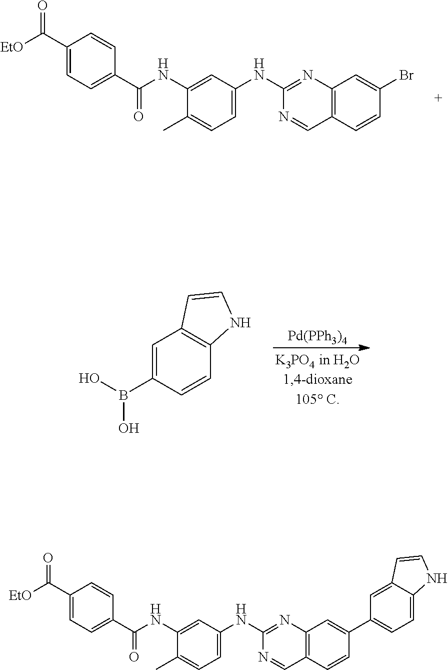 [SMILES: CCOC(=O)c1ccc(C(=O)Nc2cc(Nc3ncc4ccc(Br)cc4n3)ccc2C)cc1 ; OB(O)c1ccc2[nH]ccc2c1 ; CCOC(=O)c1ccc(C(=O)Nc2cc(Nc3ncc4ccc(-c5ccc6[nH]ccc6c5)cc4n3)ccc2C)cc1 ; "Pd(PPh3)4"]

A 0.5 mL-2.0 mL tapered microwave vial with triangular stir vane was charged with ethyl 4-((5-((7-bromoquinazolin-2-yl)amino)-2-methylphenyl)carbamoyl)benzoate (30 mg, 0.06 mmol, 1 equiv), (1H-indol-5-yl)boronic acid (14 mg, 0.09 mmol, 1.5 equiv), potassium phosphate (89 μL, 0.18 mmol, 3 equiv), and dioxane (0.6 mL, 0.1 M). Tetrakis(triphenylphosphine)palladium(0) (14 mg, 0.012 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂C12:MeOH). R^{f}: 0.6-0.7. (3 mg, 32% yield).

An ester hydrolysis then was performed:

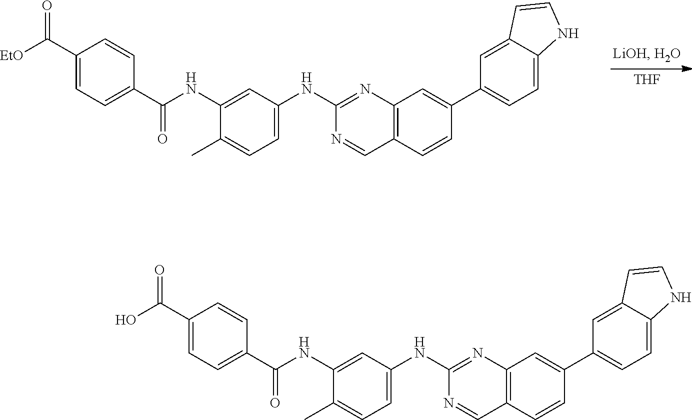 [SMILES: CCOC(=O)c1ccc(C(=O)Nc2cc(Nc3ncc4ccc(-c5ccc6[nH]ccc6c5)cc4n3)ccc2C)cc1 ; Cc1ccc(Nc2ncc3ccc(-c4ccc5[nH]ccc5c4)cc3n2)cc1NC(=O)c1ccc(C(=O)O)cc1 ; "THF"]

A 0.5-2.0 mL tapered microwave vial with triangular stir vane was charged with a suspension of ethyl 4-((5-((7-(1H-indol-5-yl)quinazolin-2-yl)amino)-2-methylphenyl)carbamoyl)benzoate (3 mg, 0.006 mmol) in THF (0.4 mL). Water (0.028 mL, 0.055 mmol) was added followed by LiOH hydrate (2.3 mg, 0.055 mmol). The vial was sealed and heated in an aluminum block (85° C.) for 12 hr. The reaction was diluted with EtOAc and 1M HCl. Layers were separated and the aqueous layer was extracted with EtOAc (3×). Organic layers were combined, dried over MgSO₄, and concentrated under reduced pressure to afford product (2.6 mg, 91% yield).

### Example 46: Preparation of Cmpd42

A Suzuki coupling process was performed:

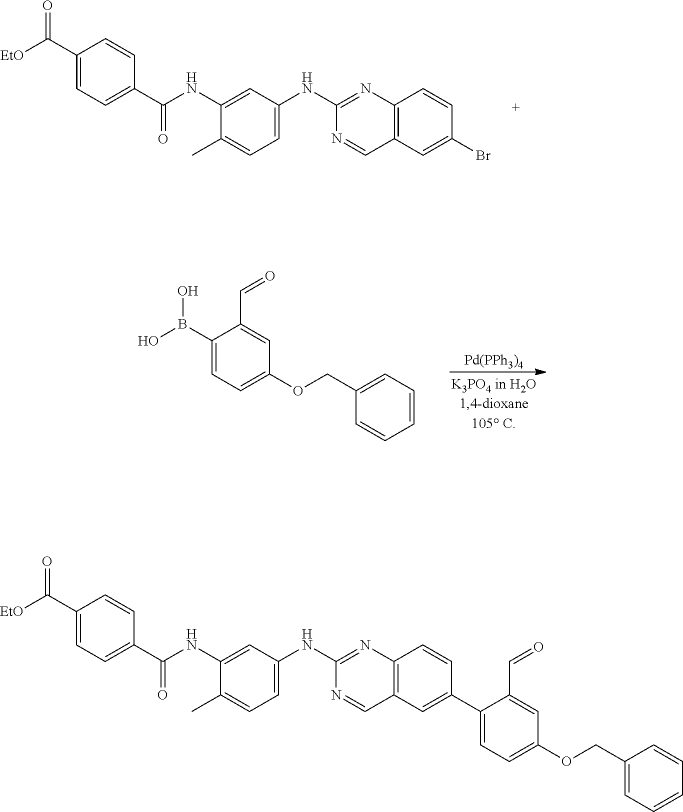 [SMILES: CCOC(=O)c1ccc(C(=O)Nc2cc(Nc3ncc4cc(Br)ccc4n3)ccc2C)cc1 ; O=Cc1cc(OCc2ccccc2)ccc1B(O)O ; CCOC(=O)c1ccc(C(=O)Nc2cc(Nc3ncc4cc(-c5ccc(OCc6ccccc6)cc5C=O)ccc4n3)ccc2C)cc1 ; "Pd(PPh3)4"]

A 0.5 mL-2.0 mL tapered microwave vial with triangular stir vane was charged with ethyl 4-((5-((6-bromoquinazolin-2-yl)amino)-2-methylphenyl)carbamoyl)benzoate (40 mg, 0.079 mmol, 1 equiv), (4-(benzyloxy)-2-formylphenyl)boronic acid (30.4 mg, 0.119 mmol, 1.5 equiv), potassium phosphate (119 μL, 0.237 mmol, 3 equiv), and dioxane (0.44 mL, 0.18 M). Tetrakis(triphenylphosphine)palladium(0) (18.3 mg, 0.016 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂Cl₂:MeOH). One band was isolated containing pure product.

An ester hydrolysis then was performed:

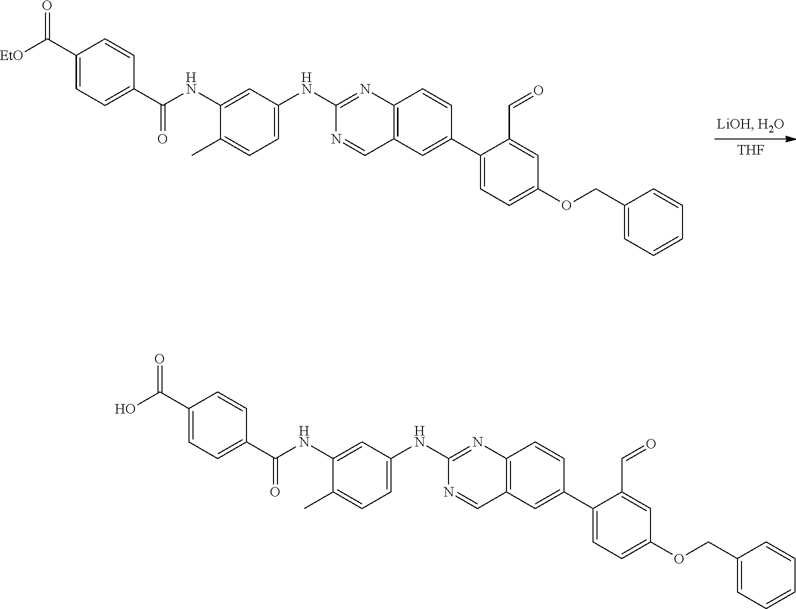 [SMILES: CCOC(=O)c1ccc(C(=O)Nc2cc(Nc3ncc4cc(-c5ccc(OCc6ccccc6)cc5C=O)ccc4n3)ccc2C)cc1 ; Cc1ccc(Nc2ncc3cc(-c4ccc(OCc5ccccc5)cc4C=O)ccc3n2)cc1NC(=O)c1ccc(C(=O)O)cc1 ; "THF"]

A 0.5-2.0 mL tapered microwave vial with triangular stir vane was charged with a suspension of ethyl 4-((5-((6-(4-(benzyloxy)-2-formylphenyl)quinazolin-2-yl)amino)-2-methylphenyl)carbamoyl)benzoate (50.4 mg, 0.08 mmol) in THF (3.96 mL). Water (0.4 mL, 0.08 mmol) was added followed by LiOH hydrate (33.2 mg, 0.08 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight to afford pure product (12.1 mg, 25% yield).

### Example 47: Preparation of Cmpd43

The title compound having the following structure was prepared:

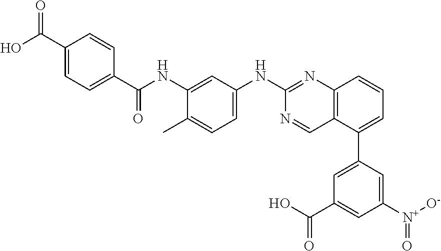 [SMILES: Cc1ccc(Nc2ncc3c(-c4cc(C(=O)O)cc([N+](=O)[O-])c4)cccc3n2)cc1NC(=O)c1ccc(C(=O)O)cc1]

A Suzuki coupling process was performed:

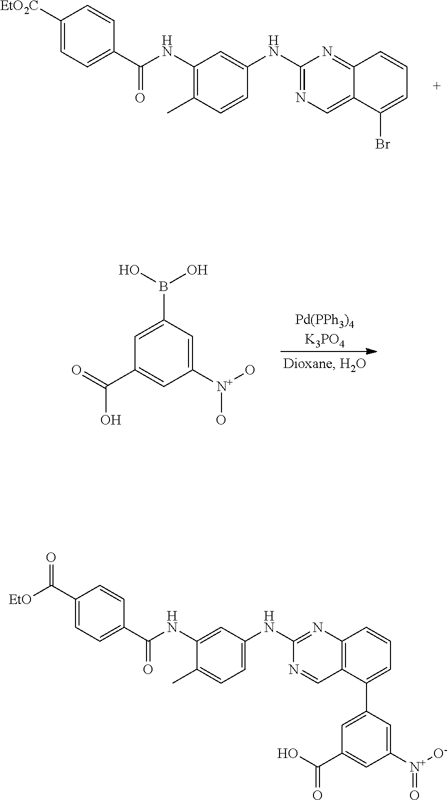 [SMILES: CCOC(=O)c1ccc(C(=O)Nc2cc(Nc3ncc4c(Br)cccc4n3)ccc2C)cc1 ; O=C(O)c1cc(B(O)O)cc([NH+](O)O)c1 ; CCOC(=O)c1ccc(C(=O)Nc2cc(Nc3ncc4c(-c5cc(C(=O)O)cc([N+](=O)[O-])c5)cccc4n3)ccc2C)cc1 ; "Pd(PPh3)4K3PO4"]

A 0.5 mL-2.0 mL tapered microwave vial with triangular stir vane was charged with ethyl 4-((5-((5-bromoquinazolin-2-yl)amino)-2-methylphenyl)carbamoyl)benzoate (100 mg, 0.198 mmol, 1 equiv), 3-Borono-5-nitrobenzoic acid (62.6 mg, 0.297 mmol, 1.5 equiv), potassium phosphate (297 μL, 0.594 mmol, 3 equiv), and dioxane (1.1 mL, 0.18 M).

Tetrakis(triphenylphosphine)palladium(0) (45.7 mg, 0.04 mmol, 0.2 equiv) were added, and the vial sealed, then heated in an aluminum block (105° C.) overnight.

The reaction was diluted with excess iPrOAc, dried over MgSO₄, and concentrated under reduced pressure. The crude material was purified by pTLC (40:1 CH₂Cl₂:MeOH). One band was isolated containing pure product (7.5 mg, 6% yield).

An ester hydrolysis then was performed:

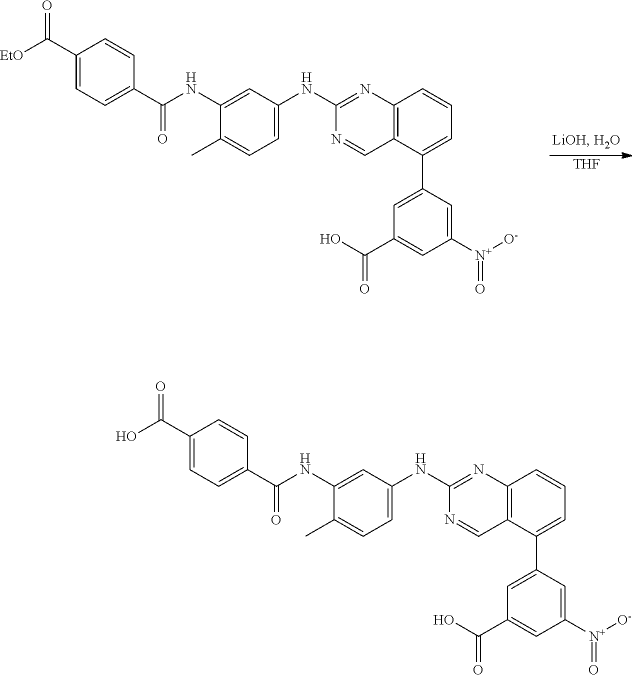

A 0.5-2.0 mL tapered microwave vial with triangular stir vane was charged with a suspension of 3-(2-((3-(4-(ethoxycarbonyl)benzamido)-4-methylphenyl)amino)quinazolin-5-yl)-5-nitrobenzoic acid (7.5 mg, 0.013 mmol) in THF (634 μL). Water (63 μL, 0.127 mmol) was added followed by LiOH hydrate (5.3 mg, 0.127 mmol). The vial was sealed and heated in an aluminum block (85° C.) for 12 hr. The reaction was diluted with 1M HCl. Solid precipitated and the HCl was removed. The material was then washed with water (3×). Water was removed via a syringe and the material was lyophilized overnight to afford pure product (0.4 mg, 6% yield).

### Example 48: Preparation of Cmpd25

A Sonogashira coupling process was performed:

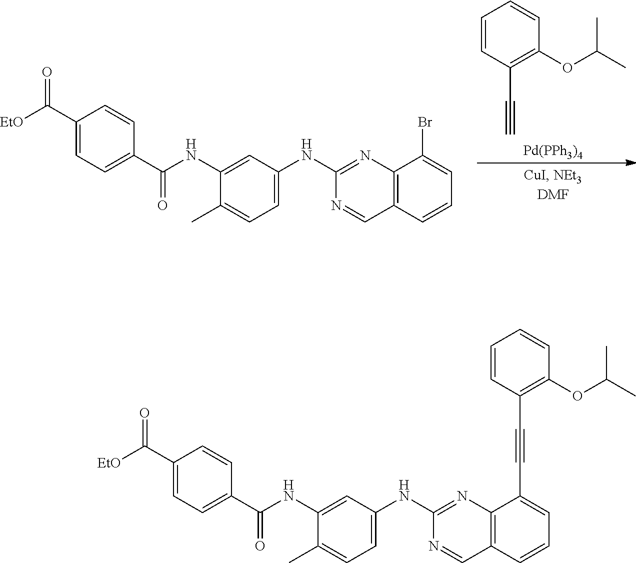 [SMILES: CCOC(=O)c1ccc(C(=O)Nc2cc(Nc3ncc4cccc(Br)c4n3)ccc2C)cc1 ; C#Cc1ccccc1OC(C)C ; CCOC(=O)c1ccc(C(=O)Nc2cc(Nc3ncc4cccc(C#Cc5ccccc5OC(C)C)c4n3)ccc2C)cc1 ; "Pd(PPh3)4"]

A 0.5 mL-2.0 mL tapered microwave vial with triangular stir vane was charged with ethyl 4-((5-((8-bromoquinazolin-2-yl)amino)-2-methylphenyl)carbamoyl)benzoate (15 mg, 0.03 mmol, 1 equiv), 1-ethynyl-2-isopropoxybenzene (6.2 mg, 0.039 mmol, 1.3 equiv), Triethylamine (6.2 μL, 0.045 mmol, 1.5 equiv), and DMF (297 μL, 0.1 M). Tetrakis(triphenylphosphane)palladium(0) (6.86 mg, 0.006 mmol) and Copper iodide (2.26 mg, 0.012 mmol, 0.4 equiv) were added, and the vial sealed, then heated in an aluminum block (60° C.) for 16 hours. The reaction was diluted with EtOAc and filtered. Crude material was purified by pTLC (40:1 DCM:MeOH).

An ester hydrolysis then was performed:

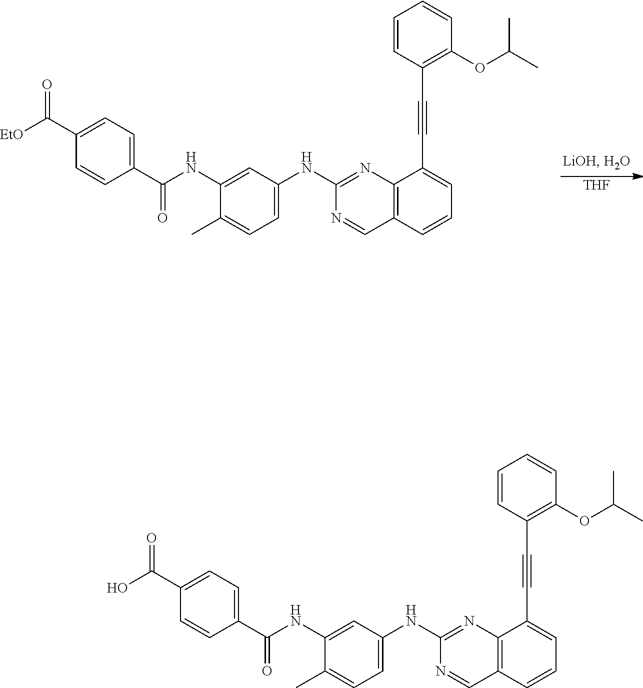

A 0.5-2.0 mL tapered microwave vial with triangular stir vane was charged with a suspension of ethyl 4-((5-((8-((2-isopropoxyphenyl)ethynyl)quinazolin-2-yl)amino)-2-methylphenyl)carbamoyl)benzoate (20.0 mg, 0.034 mmol) in THF (0.611 mL). Water (0.171 mL, 0.342 mmol) was added followed by LiOH hydrate (14.4 mg, 0.342 mmol). The vial was sealed and heated in an aluminum block (85° C.) for 12 hr. The reaction was diluted with EtOAc and 1M HCl. Layers were separated and the aqueous layer was extracted with EtOAc (3×). Organic layers were combined, dried over MgSO₄, and concentrated under reduced pressure to afford pure product.

### Example 49: Preparation of Cmpd77

A Sonogashira coupling process is performed:

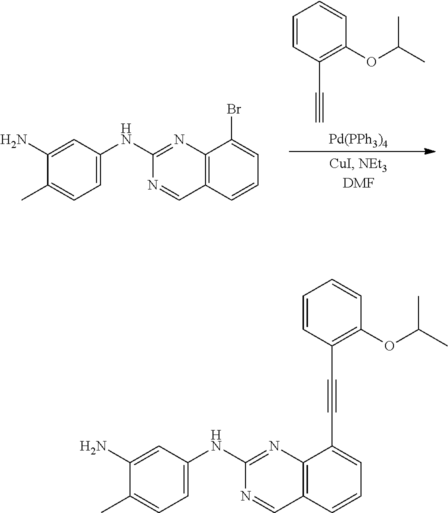 [SMILES: Cc1ccc(Nc2ncc3cccc(Br)c3n2)cc1N ; C#Cc1ccccc1OC(C)C ; Cc1ccc(Nc2ncc3cccc(C#Cc4ccccc4OC(C)C)c3n2)cc1N ; "Pd(PPh3)4"]

A 0.5 mL-2.0 mL tapered microwave vial with triangular stir vane is charged with N1-(8-bromoquinazolin-2-yl)-4-methylbenzene-1,3-diamine (15 mg, 0.046 mmol, 1 equiv), 1-ethynyl-2-isopropoxybenzene (9.5 mg, 0.059 mmol, 1.3 equiv), Triethylamine (9.5 μL, 0.068 mmol, 1.5 equiv), and DMF (456 μL, 0.1 M). Tetrakis(triphenylphosphane)palladium(0) (10.5 mg, 0.009 mmol) and Copper iodide (3.5 mg, 0.018 mmol, 0.4 equiv) are added, and the vial sealed, then heated in an aluminum block (60° C.) for 16 hours. The reaction is diluted with EtOAc and filtered. Crude material is purified by pTLC (40:1 DCM:MeOH).

### Example 50: Preparation of Cmpd78

An amidation process is performed utilizing Cmpd79 (preparation process described in Example 49) as a reactant:

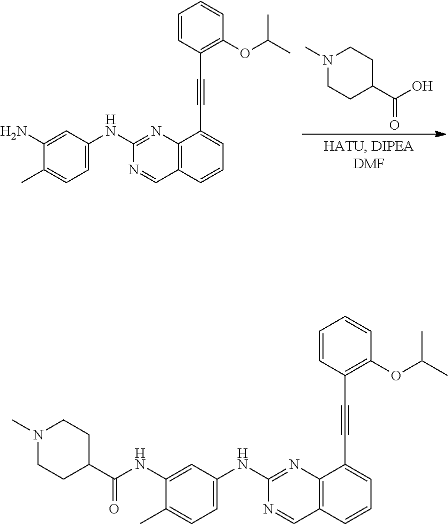

A 4 mL vial is equipped with N1-(8-((2-isopropoxyphenyl)ethynyl)quinazolin-2-yl)-4-methylbenzene-1,3-diamine (15 mg, 0.037 mmol, 1 equiv), 1-methylpiperidine-4-carboxylic acid (8 mg, 0.056 mmol, 1.5 equiv), HATU (15.5 mg, 0.041 mmol, 1.1 equiv) and DMF (730 μL, 0.05 M). Diisopropylethylamine is then added (19.2 μL, 0.110 mmol, 3 equiv) and the reaction is stirred at room temperature. The reaction is diluted with saturated aqueous NaHCO₃ and EtOAC. The aqueous layer is extracted with EtOAc (3×). The combined organic layer is washed with water to remove remaining DMF, then dried over MgSO₄, and concentrated under reduced pressure. The crude material is purified by pTLC (40:1 DCM:MeOH).

### Example 51: Preparation of Cmpd79

The titled compound having the following structure is prepared:

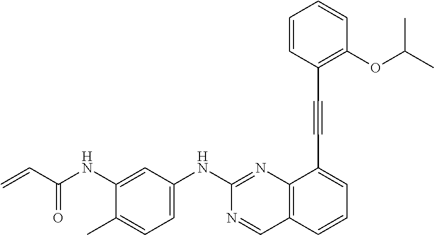 [SMILES: C=CC(=O)Nc1cc(Nc2ncc3cccc(C#Cc4ccccc4OC(C)C)c3n2)ccc1C]

An amidation process is performed:

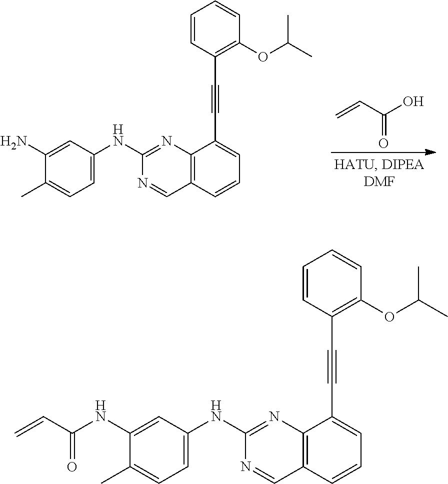

A 4 mL vial is equipped with N1-(8-((2-isopropoxyphenyl)ethynyl)quinazolin-2-yl)-4-methylbenzene-1,3-diamine (15 mg, 0.037 mmol, 1 equiv), acrylic acid (4 mg, 0.055 mmol, 1.5 equiv), HATU (15.5 mg, 0.041 mmol, 1.1 equiv) and DMF (730 μL, 0.05 M). Diisopropylethylamine is then added (19.2 μL, 0.110 mmol, 3 equiv) and the reaction is stirred at room temperature. The reaction is diluted with saturated aqueous NaHCO₃ and EtOAC. The aqueous layer is extracted with EtOAc (3×). The combined organic layer is washed with water to remove any remaining DMF, then dried over MgSO₄, and concentrated under reduced pressure. The crude material is purified by pTLC (40:1 DCM:MeOH).

### Example 52: Preparation of Cmpd82 and Cmpd67

An Fmoc protection was performed:

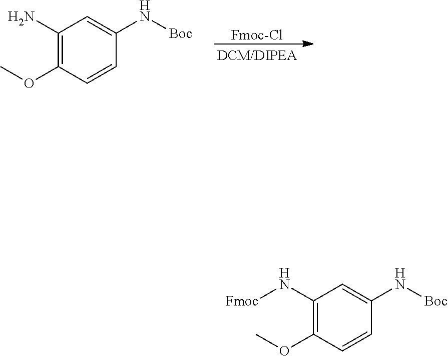

A 150 mL round bottom flask was charged with tert-butyl N-(3-amino-4-methoxy-phenyl)carbamate (1.063 g, 4.461 mmol). The dark solid was dissolved in DCM (20 mL). The dark, stirred solution was treated with DIEA (0.93 mL, 5.353 mmol) followed by solid Fmoc-Cl (1.385 g, 5.353 mmol). The solution was stirred at room temperature over a weekend. The dark reaction mixture was poured into excess iPrOAc and washed with bicarbonate (3× washes). The organic phase was dried over MgSO₄, filtered and evaporated to a heavy, black oil. The product was used without quantification or further purification.

A Boc removal process then was performed:

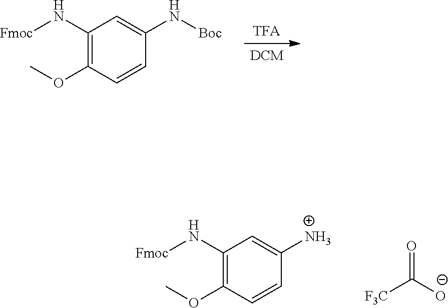

A 250 mL round bottom flask containing the crude product was treated with DCM (30 mL) and stirred until a dark, homogenous solution was obtained. TFA (6 mL) was added and the solution was stirred at room temperature for 3 hr. LCMS showed the Boc removal process was complete. Toluene (15 mL) was added and the solution evaporated to a dark, viscous residue. The residue was dissolved in iPrOAc and stirred at room temperature overnight. A small amount of light grey solid was recovered by filtration. This solid had little product and was discarded. The remaining filtrate was returned to the round bottom flask and treated with aqueous bicarbonate, then vigorously stirred for 15 min. The mixture was partitioned and the organic phase washed with water (1× wash). The water was removed and the wet organic transferred to another round bottom flask. 1N HCl (approximately 30 mL) was added, causing a precipitate to form. The two-phase mixture was vigorously stirred for 2 hr. The solid was recovered by filtration and allowed to stand under lyophilizer vacuum in the filter funnel, affording a light grey solid (1.325 g).

A nucleophilic aromatic substitution (S_{N}Ar) then was performed:

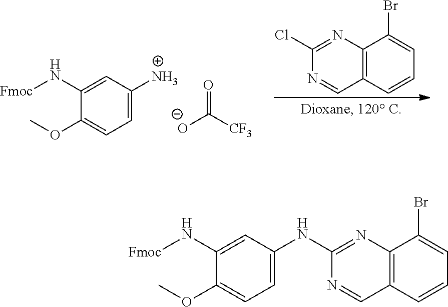

A 10-20 mL microwave vial was charged with dioxane (15 mL). With vigorous stirring, [3-(9H-fluoren-9-ylmethoxycarbonylamino)-4-methoxy-phenyl]ammonium;chloride (1.325 g, 3.339 mmol) was added. Stirring was continued and 8-bromo-2-chloro-quinazoline (1.626 g, 6.677 mmol) was added. The vial was sealed and heated in an aluminum block (125° C.) for 2 hr. LCMS showed the reaction was not complete, and the vial was resealed and heated for another 2 hr. LCMS showed the reaction was complete. The dark reaction was diluted with excess iPrOAc, affording a light yellow precipitate. The precipitate was vigorously stirred over a weekend, then recovered by filtration and allowed to stand under lyophilizer vacuum overnight, yielding a light brown powdery solid (1.971 g). LCMS showed the solid was nearly identical to the reaction mixture. The solid was suspended in DCM (approximately 100 mL) and vigorously stirred for 1 hr. The finely divided yellow solid was recovered by filtration and LCMS showed it was the desired product (513 mg).

A Suzuki coupling/deprotection process then was performed:

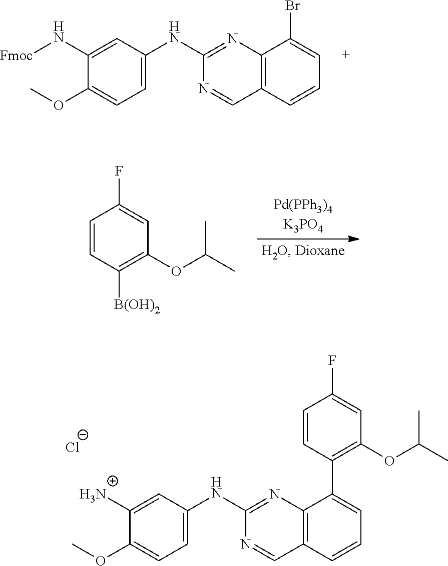

A 10-20 mL microwavable vial was charged with 9H-fluoren-9-ylmethyl N-[5-[(8-bromoquinazolin-2-yl)amino]-2-methoxy-phenyl]carbamate (513 mg, 0.904 mmol). Dioxane (10 mL) was added and the mixture stirred for 5 min to effect complete suspension. (4-fluoro-2-isopropoxy-phenyl)boronic acid (269 mg, 1.356 mmol) was added. Aqueous K₃PO₄ (1.40 mL, 2M, 2.8 mmol) was added followed by solid K₃PO₄ (192 mg, 0.904 mmol). Tetrakistriphenylphosphine palladium (209 mg, 0.181 mmol) was added and the vial was sealed, then heated in an aluminum block (107° C.) for 16 hr. LCMS showed the reaction was complete. Two peaks of identical mass are observed. The dark reaction mixture was poured into excess iPrOAc (approximately 100 mL). Water (approximately 15 mL) was added and the mixture vigorously stirred for 10 min. The two-phase mixture, containing some insoluble dark material floating at the phase interface, was placed into a 250 mL separatory funnel and allowed to separate for 15 min. The mixture contained a clear, brown organic and a clear colorless aqueous with the insoluble black material suspended in the aqueous phase. The aqueous phase, along with some of the insoluble black material, was drawn off. Water (20 mL) was added and the mixture vigorously shaken, then allowed to separate for 15 min. The aqueous phase, along with the suspended black material, was carefully drawn off and set aside. The remaining organic phase was transferred to a 250 mL Ehrlenmeyer flask and combined with 1N HCl (50 mL). The two-phase mixture was vigorously stirred for 30 min. The resultant mixture contained of a clear, yellow aqueous and a turbid organic phase with a suspended cream colored insoluble solid. The mixture was transferred to a separatory funnel and allowed to separate for 15 min. The clear aqueous phase was slowly drawn off to avoid taking any insoluble material. The remaining organic phase was returned to the flask and stirred with 1N HCl (approximately 10 mL). The mixture was stirred for 10 min, then partitioned as described above. The combined aqueous and the organic phases were sampled for LCMS. The desired product was in the aqueous. The aqueous phase was transferred into a 500 mL round bottom flask and stirred with EtOAc (approximately 75 mL). Solid NaHCO₃ was added in portions until gas evolution ceased. The 2-phase mixture, composed of a clear, colorless aqueous phase and a yellow organic phase, was partitioned. The organic phase was dried over MgSO₄, filtered and evaporated to a yellow/brown solid (341 mg free base; 90.2% yield) designated as Cmpd82.

An amidation process then was performed using the product of the preceding Suzuki coupling/deprotection process (Cmpd82) as a reactant:

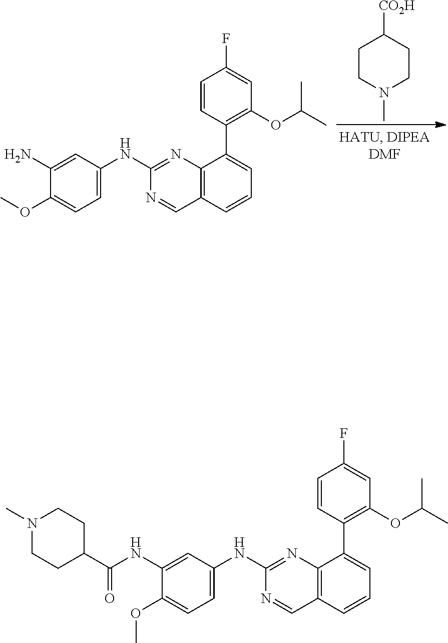

A 2.0-5.0 mL microwave vial was charged with a solution of N1-[8-(4-fluoro-2-isopropoxy-phenyl)quinazolin-2-yl]-4-methoxy-benzene-1,3-diamine (149 mg, 0.356 mmol) in DMF (2 mL). 1-Methylpiperidine-4-carboxylic acid (76 mg, 0.534 mmol) was added, followed by HATU (203 mg, 0.534 mmol). The dark solution was stirred at room temperature. LCMS at 3 hr showed the reaction was complete. The dark-colored mixture was diluted with iPrOAc (approximately 20 mL) and washed with aqueous bicarbonate (4× washes). The clear, brown organic phase was dried over MgSO₄ and filtered. The filtrate was treated with 1N HCl (15 mL) and vigorously stirred for 1 hour. The mixture was partitioned and the yellow aqueous phase was evaporated to about 15% of its initial volume on a roto-evaporator, then allowed to stand under lyophilizer vacuum overnight. A red oil was obtained that was overweight. The crude product was partitioned between iPrOAc and aqueous bicarbonate. The organic phase was dried over MgSO₄, filtered and evaporated to a dark oil. The desired product was obtained by pTLC.

### Example 53: Preparation of Cmpd54

An amidation process was performed using Cmpd82 as a reactant:

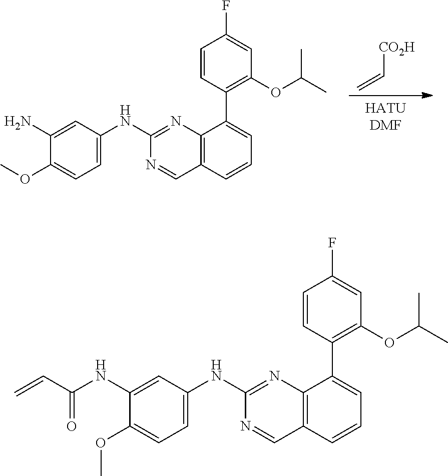

A 2.0-5.0 mL microwave vial was charged with a solution of N1-[8-(4-fluoro-2-isopropoxy-phenyl)quinazolin-2-yl]-4-methoxy-benzene-1,3-diamine (149 mg, 0.356 mmol) in DMF (2 mL). Acrylic acid (38 mg, 0.534 mmol) was added, followed by HATU (203 mg, 0.534 mmol). The dark solution was stirred at room temperature for 24 hr. LCMS showed the reaction was about 75% complete. Acrylic acid (30 mg) and HATU (100 mg) were added and the solution stirred at room temperature another 24 hr. LCMS showed the reaction was complete. The reaction mixture was partitioned between excess iPrOAc and aqueous bicarbonate. The dark organic phase was washed with water (3× washes), dried over MgSO₄, filtered and evaporated to a dark oil. The crude product was purified by pTLC.

### Example 54: Preparation of Cmpd80

An amidation process was performed using Cmpd51 (preparation process described in Example 24) as a reactant:

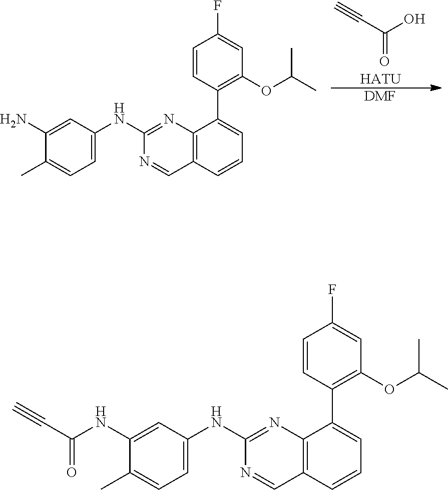

A 0.5-2.0 mL microwave vial was charged with a solution of N1-[8-(4-fluoro-2-isopropoxy-phenyl)quinazolin-2-yl]-4-methyl-benzene-1,3-diamine (86 mg, 0.214 mmol). DMF (1 mL) was added and the mixture stirred at room temperature until dissolution was complete. Prop-2-ynoic acid (22 mg, 0.321 mmol) was added, followed by HATU (122 mg, 0.321 mmol). The dark solution was stirred at room temperature for 18 hr. LCMS showed the reaction at about 50% complete based on parent ion abundance. Prop-2-ynoic acid (22 mg) and HATU (100 mg) were added and the solution stirred at room temperature overnight. LCMS showed no SM mass detected. The reaction mixture was added to excess iPrOAc and washed with aqueous bicarbonate (4× washes). The organic phase was dried over MgSO₄, filtered and evaporated to a dark oil. The oil was purified by pTLC (8:2:1 Hexane:EtOAc:MeOH) to afford the desired product (9.8 mg isolated, 10% yield).

### Example 55: Preparation of Cmpd33

A Suzuki coupling process was performed:

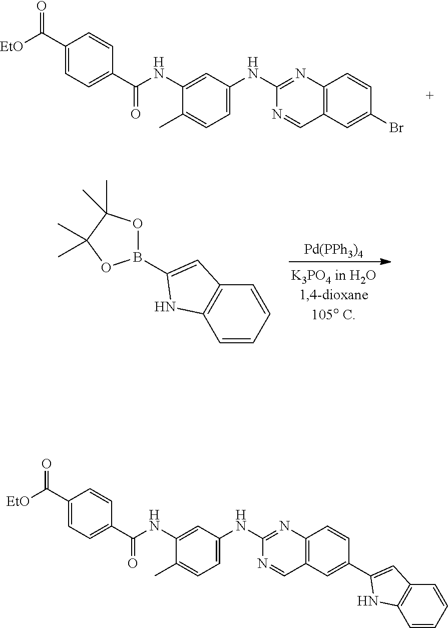 [SMILES: CCOC(=O)c1ccc(C(=O)Nc2cc(Nc3ncc4cc(Br)ccc4n3)ccc2C)cc1 ; CC1(C)OB(c2cc3ccccc3[nH]2)OC1(C)C ; CCOC(=O)c1ccc(C(=O)Nc2cc(Nc3ncc4cc(-c5cc6ccccc6[nH]5)ccc4n3)ccc2C)cc1 ; "Pd(PPh3)4"]

A 0.5 mL-2.0 mL tapered microwave vial with triangular stir vane was charged with ethyl 4-((5-((6-bromoquinazolin-2-yl)amino)-2-methylphenyl)carbamoyl)benzoate (100 mg, 0.2 mmol, 1 equiv), 2-(4,4,5,5-tetramethyl-1,3,2-dioxaborolan-2-yl)-1H-indole (72 mg, 0.3 mmol, 1.5 equiv), potassium phosphate (300 μL, 0.594 mmol, 3 equiv), and dioxane (1.1 mL). Tetrakis(triphenylphosphine)palladium(0) (45.7 mg, 0.04 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂Cl₂:MeOH). One band was isolated containing product. The material was taken on to the next step.

An ester hydrolysis then was performed:

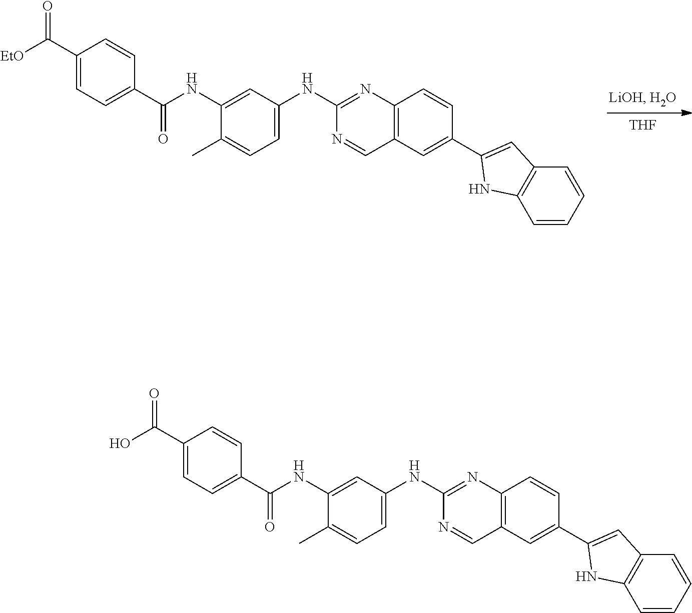

A 0.5-2.0 mL tapered microwave vial with triangular stir vane was charged with a suspension of ethyl 4-((5-((6-(1H-indol-2-yl)quinazolin-2-yl)amino)-2-methyl phenyl)carbamoyl)benzoate (4.8 mg, 0.08 mmol) in THF (0.45 mL). Water (0.044 mL, 0.09 mmol) was added followed by LiOH hydrate (3.7 mg, 0.0 m mmol). The vial was sealed and heated in an aluminum block (85° C.) for 12 hr. The reaction was diluted with 1M HCl and washed with EtOAc (3×). The combined organic layers were dried over MgSO₄ and concentrated under reduced pressure to yield the product.

### Example 56: Preparation of Cmpd34

A Suzuki coupling process was performed:

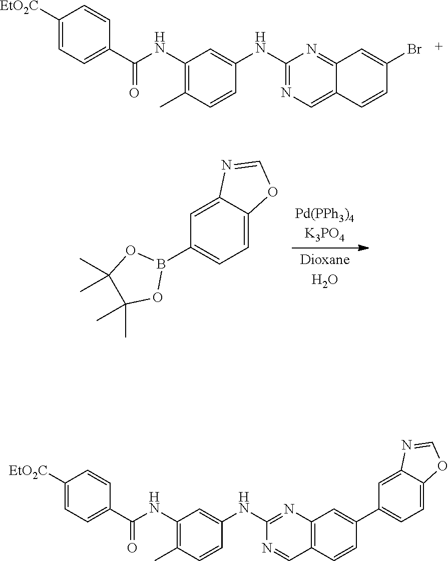 [SMILES: CCOC(=O)c1ccc(C(=O)Nc2cc(Nc3ncc4ccc(Br)cc4n3)ccc2C)cc1 ; CC1(C)OB(c2ccc3ocnc3c2)OC1(C)C ; CCOC(=O)c1ccc(C(=O)Nc2cc(Nc3ncc4ccc(-c5ccc6ocnc6c5)cc4n3)ccc2C)cc1]

A 0.5 mL-2.0 mL tapered microwave vial with triangular stir vane was charged with ethyl 4-((5-((7-bromoquinazolin-2-yl)amino)-2-methylphenyl)carbamoyl)benzoate (30 mg, 0.059 mmol, 1 equiv), 5-(4,4,5,5-Tetramethyl-1,3,2-dioxaborolan-2-yl)benzo[d]oxazole (22 mg, 0.089 mmol, 1.5 equiv), potassium phosphate (89 μL, 2M, 0.178 mmol, 3 equiv), and dioxane (0.6 mL).

Tetrakis(triphenylphosphine)palladium(0) (13.7 mg, 0.012 mmol, 0.2 equiv) was added, and the vial sealed, then heated in an aluminum block (105° C.) overnight. The reaction was diluted with excess iPrOAc, dried over MgSO₄, and concentrated under reduced pressure. The crude material was purified by pTLC (40:1 CH₂Cl₂:MeOH). One band (rf; 0.6) was isolated containing product.

An ester hydrolysis then was performed:

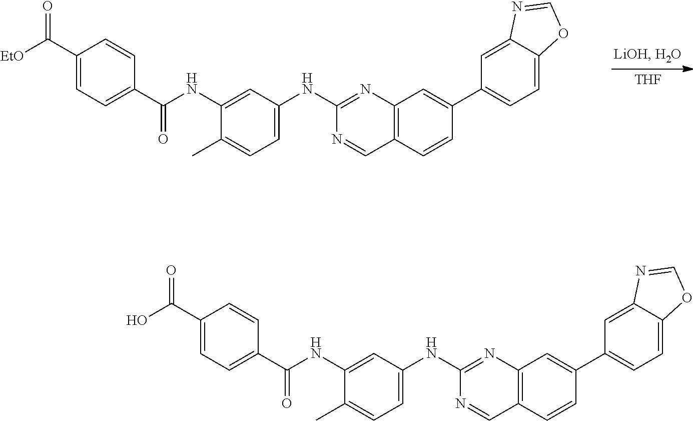 [SMILES: CCOC(=O)c1ccc(C(=O)Nc2cc(Nc3ncc4ccc(-c5ccc6ocnc6c5)cc4n3)ccc2C)cc1 ; Cc1ccc(Nc2ncc3ccc(-c4ccc5ocnc5c4)cc3n2)cc1NC(=O)c1ccc(C(=O)O)cc1 ; "THF"]

A 0.5-2.0 mL tapered microwave vial with triangular stir vane was charged with a suspension of ethyl 4-((5-((7-(benzo[d]oxazol-5-yl)quinazolin-2-yl)amino)-2-methylphenyl)carbamoyl)benzoate (12.3 mg, 0.023 mmol) in THF (0.45 mL). Water (0.113 mL, 0.226 mmol) was added followed by LiOH hydrate (9.5 mg, 0.226 mmol). The vial was sealed and heated in an aluminum block (85° C.) for 12 hr. The reaction was diluted with 1M HCl then washed with EtOAc (3×). The combined organic layers were dried over MgSO₄ and concentrated under reduced pressure to yield the product.

### Example 57: Preparation of Cmpd39

A Suzuki coupling process was performed:

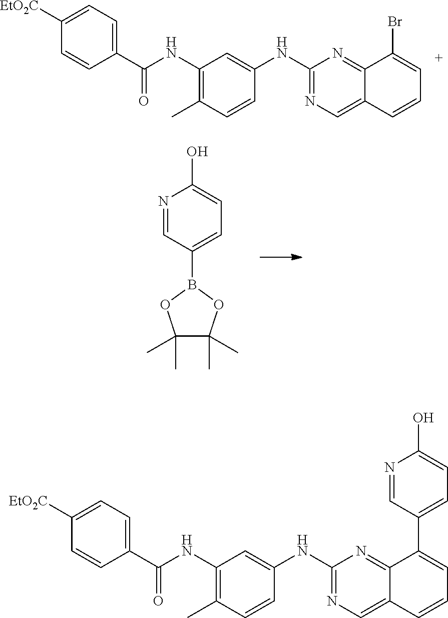

A 0.5 mL-2.0 mL tapered microwave vial with triangular stir vane was charged with ethyl 4-((5-((7-bromoquinazolin-2-yl)amino)-2-methylphenyl)carbamoyl)benzoate (35 mg, 0.07 mmol, 1 equiv), 5-(4,4,5,5-Tetramethyl-1,3,2-dioxaborolan-2-yl)pyridin-2-ol (23 mg, 0.1 mmol, 1.5 equiv), potassium phosphate (104 μL, 0.21 mmol, 3 equiv), and dioxane (0.39 mL). Tetrakis(triphenylphosphine)palladium(0) (16 mg, 0.014 mmol, 0.2 equiv) was added, and the vial sealed, then heated in an aluminum block (105° C.) overnight. The reaction was diluted with excess iPrOAc and dried over MgSO₄. Filtration and evaporation afforded a yellow solid. The crude material was purified by pTLC (40:1 CH₂Cl₂:MeOH). One band was isolated containing product (12 mg, 33% yield).

An ester hydrolysis then was performed:

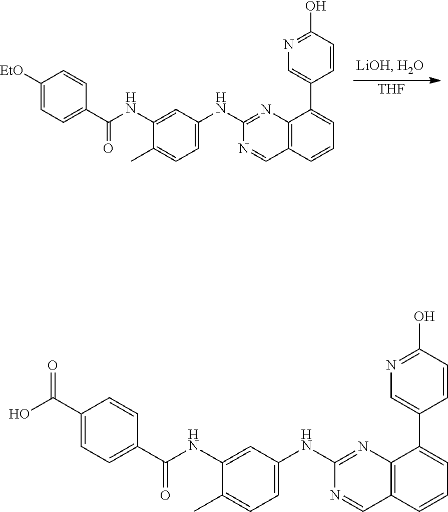 [SMILES: CCOc1ccc(C(=O)Nc2cc(Nc3ncc4cccc(-c5ccc(O)nc5)c4n3)ccc2C)cc1 ; Cc1ccc(Nc2ncc3cccc(-c4ccc(O)nc4)c3n2)cc1NC(=O)c1ccc(C(=O)O)cc1 ; "THF"]

A 0.5-2.0 mL tapered microwave vial with triangular stir vane was charged with a suspension of ethyl 4-((5-((8-(6-hydroxypyridin-3-yl)quinazolin-2-yl)amino)-2-methylphenyl)carbamoyl)benzoate (20.1 mg, 0.039 mmol) in THF (3.6 mL). Water (0.2 mL, 0.39 mmol) was added followed by LiOH hydrate (16 mg, 0.39 mmol). The vial was sealed and heated in an aluminum block (85° C.) for 12 hr. The reaction was diluted with 1M HCl. A solid precipitated and the HCl was removed by syringe. The material was then washed with water (3×). As much water as possible was removed via syringe and the material was allowed to stand under lyophilizer vacuum overnight, affording the product.

### Example 58: Preparation of Cmpd81

An amidation process was performed:

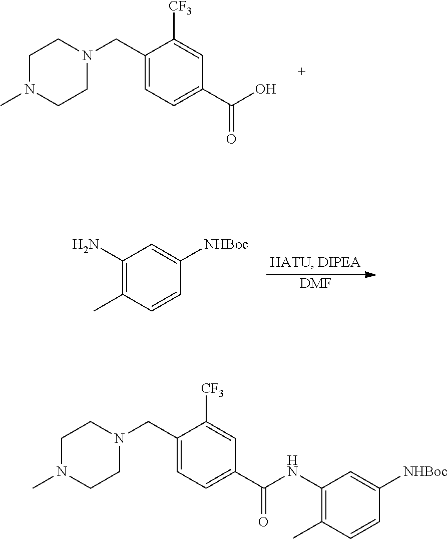

A 20 mL vial was charged with 4-((4-methylpiperazin-1-yl)methyl)-3-(trifluoromethyl)benzoic acid (200 mg, 0.66 mmol, 1 equiv), tert-butyl (3-amino-4-methylphenyl)carbamate (220 mg, 0.99 mmol, 1.5 equiv), HATU (276 mg, 0.73 mmol, 1.1 equiv) and DMF (4.4 mL). Diisopropylamine was then added (345 μL, 0.109 mmol, 3 equiv) and the reaction was stirred at room temperature overnight. The reaction was diluted with EtOAc and H₂O. The layers were separated, and the aqueous layer was extracted with EtOAc (3×). The combined organic layers were washed with H₂O to remove any remaining DMF, then dried over MgSO₄, and concentrated under reduced pressure. The crude product was used in the next step without further purification.

A Boc removal process then was performed:

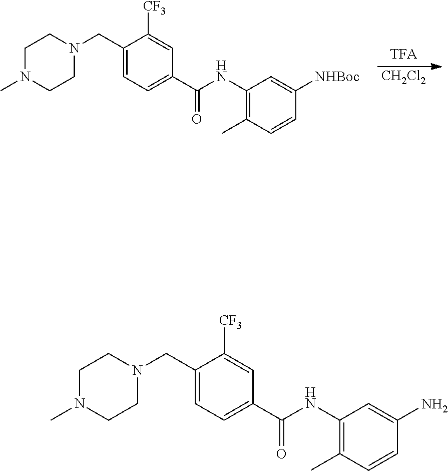

A solution of tert-butyl (4-methyl-3-(4-((4-methylpiperazin-1-yl)methyl)-3-(trifluoromethyl)benzamido)phenyl)carbamate (335 mg, 0.66 mmol) in DCM (6.6 mL) was treated with TFA (1.4 mL) at room temperature. The homogenous yellow reaction was stirred for 1 hr and sampled for LCMS, which shoed the reaction was complete. Toluene (10 mL) was added and the solution evaporated to a residue. The residue was partitioned between iPrOAc and saturated aqueous sodium bicarbonate (2× washes). The organic phase was dried over MgSO₄, filtered and evaporated to a yellow oil (238 mg) which became a tacky, cream colored solid on standing. The material (164.3 mg) was used without further purification.

A nucleophilic aromatic substitution (S_{N}Ar) then was performed:

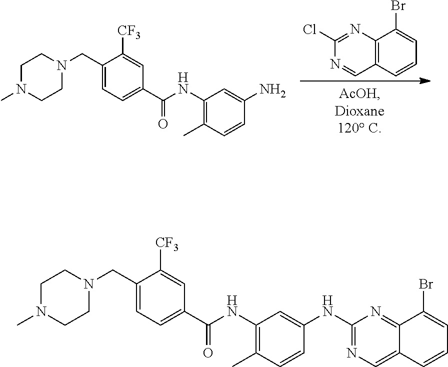 [SMILES: Cc1ccc(N)cc1NC(=O)c1ccc(CN2CCN(C)CC2)c(C(F)(F)F)c1 ; Clc1ncc2cccc(Br)c2n1 ; Cc1ccc(Nc2ncc3cccc(Br)c3n2)cc1NC(=O)c1ccc(CN2CCN(C)CC2)c(C(F)(F)F)c1]

A 0.5-2.0 mL tapered microwave vial with triangular stir vane was charged with N-(5-amino-2-methylphenyl)-4-((4-methylpiperazin-1-yl)methyl)-3-(trifluoromethyl)benzamide (65 mg, 0.16 mmol). AcOH (0.08 mL) and 1,4-dioxane (0.72 mL) were added and the mixture stirred until significant dissolution was obtained. 8-bromo-2-chloro-quinazoline (46.8 mg, 0.19 mmol) was added. The vial was sealed and heated with stirring in an aluminum block (120° C.) overnight. The reaction was poured into excess iPrOAc and washed with sat aq sodium bicarbonate (3×). The organic layer was dried over MgSO₄, filtered and concentrated under reduced pressure. The crude product was purified by pTLC (40:1 DCM:MeOH). The band with the product was isolated (rf: 0.4) and used for the next reaction (brown solid, 3.8 mg).

A Suzuki coupling process then was performed:

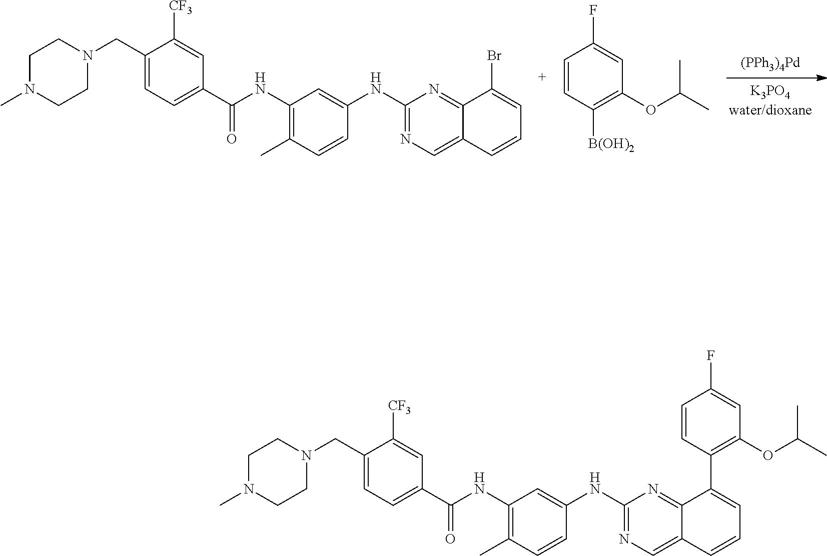

A 0.5 mL-2.0 mL tapered microwave vial with triangular stir vane was charged with N-(5-((8-bromoquinazolin-2-yl)amino)-2-methylphenyl)-4-((4-methylpiperazin-1-yl)methyl)-3-(trifluoromethyl)benzamide (20 mg, 0.032 mmol, 1 equiv), 2-isopropoxy-4-fluorophenyl boronic acid (9.6 mg, 0.048 mmol, 1.5 equiv), K₃PO₄ (48 μL, 2M, 0.096 mmol, 3 equiv), and dioxane (250 μL). Tetrakis(triphenylphosphine)palladium (7.2 mg, 0.006 mmol, 0.20 equiv) was added, and the vial sealed, then heated in an aluminum block (105° C.) for 16 hours. LCMS indicated that the starting material was consumed. The reaction mixture was diluted with EtOAc, dried over MgSO₄, filtered, and concentrated under reduced pressure. The crude material was purified by pTLC (40:1 Dichloromethane:Methanol). One spot was isolated, and LCMS confirmed pure product (Spot C, Rf=0.2, 4.4 mg, 20% yield).

### Example 60: Preparation of Cmpd47

A Suzuki coupling process was performed:

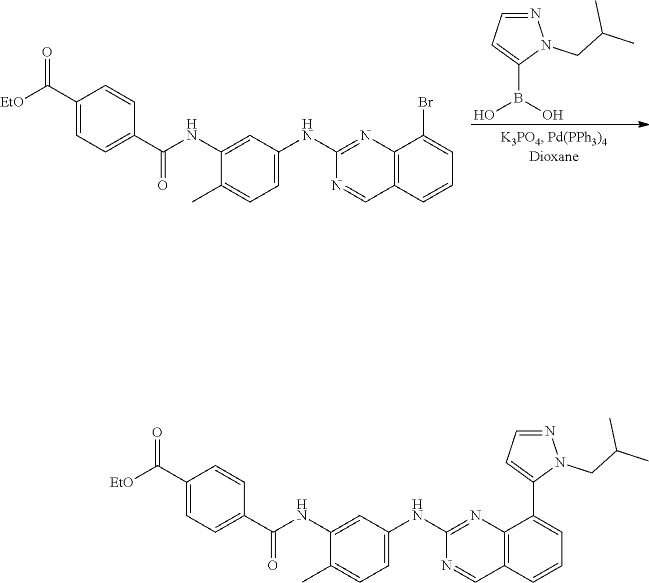 [SMILES: CCOC(=O)c1ccc(C(=O)Nc2cc(Nc3ncc4cccc(Br)c4n3)ccc2C)cc1 ; CC(C)Cn1nccc1B(O)O ; CCOC(=O)c1ccc(C(=O)Nc2cc(Nc3ncc4cccc(-c5ccnn5CC(C)C)c4n3)ccc2C)cc1]

A 0.5 mL-2.0 mL tapered microwave vial with triangular stir vane was charged with ethyl 4-((5-((8-bromoquinazolin-2-yl)amino)-2-methylphenyl)carbamoyl)benzoate (162 mg, 0.321 mmol, 1 equiv), (1-isobutyl-1H-pyrazol-5-yl)boronic acid (80.78 mg, 0.48 mmol, 1.5 equiv), K₃PO₄ (481 μL, 2M, 0.96 mmol, 3 equiv), and dioxane (1.73 mL, 0.185 M). Tetrakis(triphenylphosphine)palladium (80.1 mg, 0.07 mmol) was added, and the vial sealed, then heated in an aluminum block (105° C.) for 16 hours. The material was diluted in EtOAc and washed 3× with bicarb, dried with MgSO₄, filtered and concentrated under reduced pressure. The crude material was purified by pTLC (40:1 CH₂Cl₂:MeOH). One band was isolated containing pure product (65 mg isolated, 37% yield, Rf: 0.5).

An ester hydrolysis then was performed:

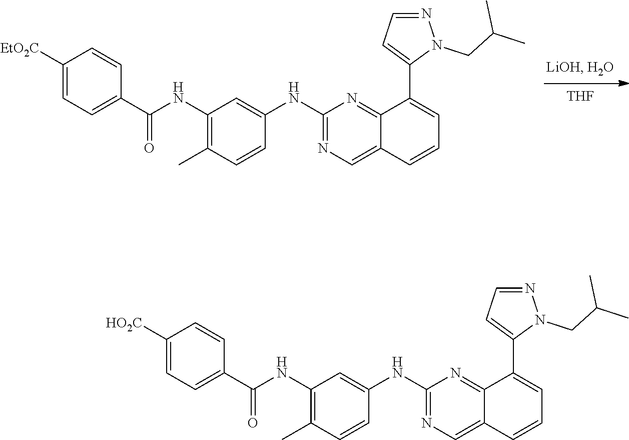 [SMILES: CCOC(=O)c1ccc(C(=O)Nc2cc(Nc3ncc4cccc(-c5ccnn5CC(C)C)c4n3)ccc2C)cc1 ; Cc1ccc(Nc2ncc3cccc(-c4ccnn4CC(C)C)c3n2)cc1NC(=O)c1ccc(C(=O)O)cc1]

A 0.5-2.0 mL tapered microwave vial with triangular stir vane was charged with a suspension of ethyl 4-((5-((8-(1-isobutyl-1H-pyrazol-5-yl)quinazolin-2-yl)amino)-2-methylphenyl)carbamoyl)benzoate (41 mg, 0.12 mmol) in THF (2.12 mL). Water (592 microliters, 1.19 mmol) was added followed by LiOH hydrate (49.7 mg, 1.19 mmol). The vial was sealed and heated in an aluminum block (85° C.) for 6 hr. The reaction was diluted with EtOAc and 1M HCl. The layers were separated and the aqueous layer was extracted with EtOAc (3×). The organic layers were combined and dried over MgSO₄. Filtration and evaporation afforded the crude material which was purified by pTLC (40:1 DCM:MeOH) to afford the product (38.3 mg, 62% yield).

An amidation process then was performed:

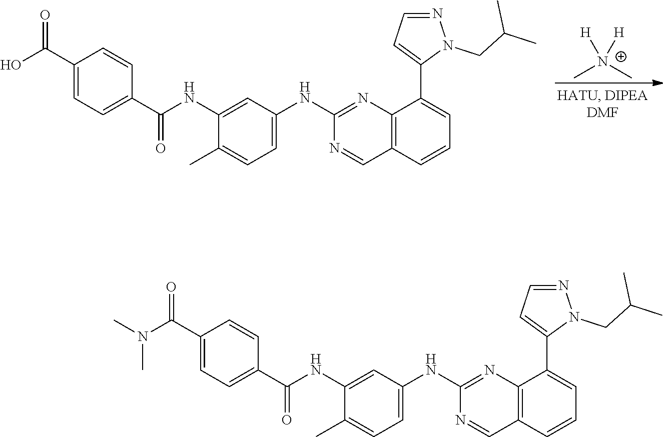

A 4 mL vial was charged with 4-((5-((8-(1-isobutyl-1H-pyrazol-5-yl)quinazolin-2-yl)amino)-2-methylphenyl)carbamoyl)benzoic acid (9.3 mg, 0.018 mmol, 1 equiv), dimethylamine hydrochloride (2.19 mg, 0.027 mmol, 1.5 equiv), HATU (7.5 mg, 0.020 mmol, 1.1 equiv) and DMF (357 μL, 0.05 M). Diisopropylethylamine was then added (9.3 μL, 0.054 mmol, 3 equiv) and the reaction was stirred at room temperature overnight. After 16 hr the reaction was incomplete according to LCMS. Additional dimethylamine hydrochloride (2.19 mg, 0.027 mmol, 1.5 equiv), HATU (7.5 mg, 0.020 mmol, 1.1 equiv), and DIPEA (9.3 μL, 0.054 mmol, 3 equiv) were added and the reaction was allowed to stir overnight. The reaction was diluted with sat. aq. NaHCO₃ and EtOAC. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (4 mg, 41% yield).

A nucleophilic aromatic substitution (S_{N}Ar) then was performed:

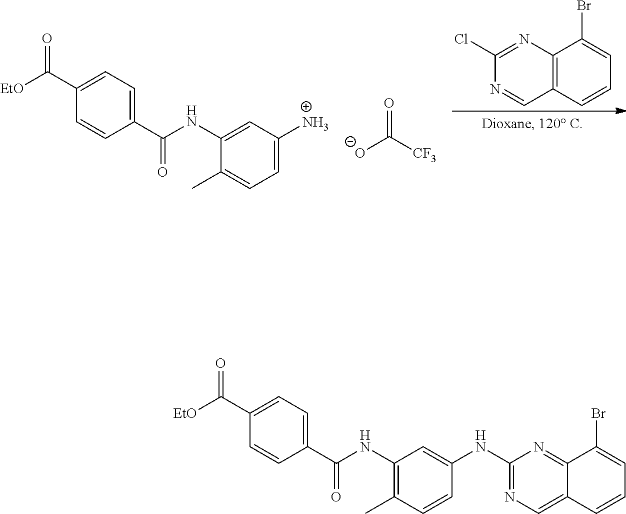 [SMILES: CCOC(=O)c1ccc(C(=O)Nc2cc([NH3+])ccc2C)cc1 ; O=C([O-])C(F)(F)F ; Clc1ncc2cccc(Br)c2n1 ; CCOC(=O)c1ccc(C(=O)Nc2cc(Nc3ncc4cccc(Br)c4n3)ccc2C)cc1]

A 5.0 mL tapered microwave vial with triangular stir vane was charged with 3-(4-(ethoxycarbonyl)benzamido)-4-methylbenzenaminium 2,2,2-trifluoroacetate (155 mg, 0.38 mmol). Dioxane (1.88 mL) was added and the mixture stirred until significant dissolution was obtained. 8-bromo-2-chloro-quinazoline (183 mg, 0.75 mmol) was added. The vial was sealed and heated with stirring in an aluminum block (120° C.) for 1 hour. After 1 hour, the reaction was allowed to cool to room temperature, then diluted with additional dioxane (2 mL). The solid was mixed with a spatula and the suspension was allowed to stir for 1 hr at room temperature. The supernatant was then filtered and the resulting solid dried overnight via lyophilization (65 mg obtained, 34% yield).

### Example 61: Preparation of Cmpd48

An amidation process was performed using an intermediate described in Example 60 as a reactant:

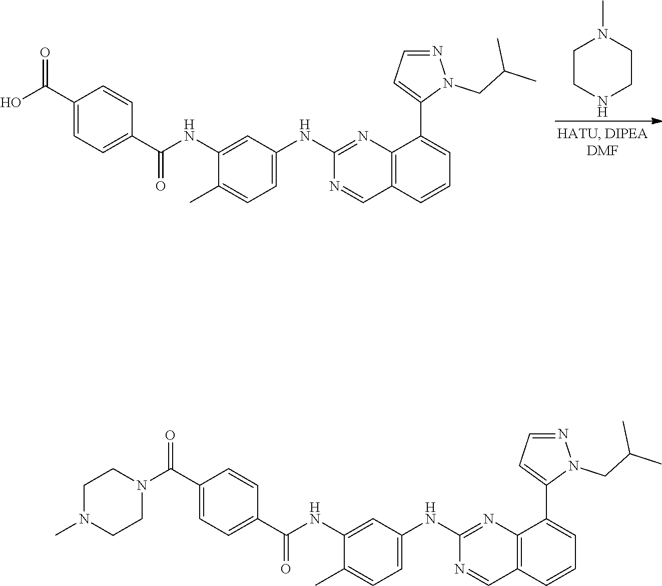

A 4 mL vial was equipped with 4-((5-((8-(1-isobutyl-1H-pyrazol-5-yl)quinazolin-2-yl)amino)-2-methylphenyl)carbamoyl)benzoic acid (16.2 mg, 0.031 mmol, 1 equiv), 1-methylpiperazine (5.2 μL, 0.047 mmol, 1.5 equiv), HATU (13 mg, 0.034 mmol, 1.1 equiv) and DMF (0.622 mL, 0.05 M). Diisoproethylpylamine was then added (16.2 μL, 0.093 mmol, 3 equiv) and the reaction was stirred at room temperature overnight. The reaction was diluted with sat. aq. NaHCO₃ and EtOAc. The aqueous phase was extracted with EtOAc (3×). The combined organic layer was washed with water to remove any remaining DMF, then dried over MgSO₄ and concentrated under reduced pressure. The crude material was purified by pTLC (40:1 DCM:MeOH), yielding pure product (3.2 mg, 17% yield).

### Example 62: Preparation of Cmpd49

An amidation process was performed:

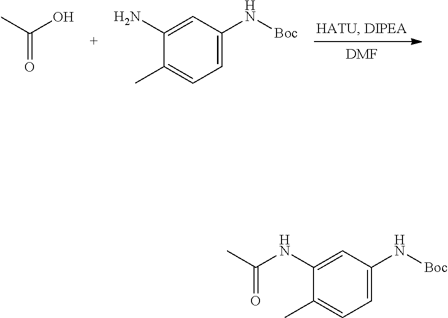

A 4 mL vial was charged with tert-butyl (3-amino-4-methylphenyl)carbamate (100 mg, 0.045 mmol, 1 equiv), acetic acid (40 μL, 0.675 mmol, 1.5 equiv), HATU (188.16 mg, 0.495 mmol, 1.1 equiv) and DMF (3 mL, 0.15 M). Diisopropylethylamine was then added (235 μL, 1.35 mmol, 3 equiv) and the reaction was stirred at room temperature overnight. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was carried on to the next reaction.

A Boc removal process then was performed:

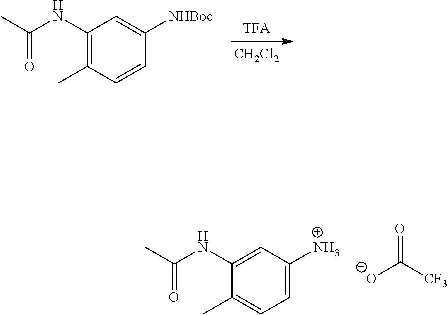 [SMILES: CC(=O)Nc1cc(NC(=O)OC(C)(C)C)ccc1C ; CC(=O)Nc1cc([NH3+])ccc1C ; O=C([O-])C(F)(F)F]

A solution of tert-butyl (3-acetamido-4-methylphenyl)carbamate (118 mg, 0.45 mmol) in DCM (3.57 mL) was treated with TFA (0.89 mL) at room temperature. The homogenous yellow reaction was stirred for 1 hr and sampled for LCMS. Toluene (10 mL) was added and the solution evaporated to a residue. The residue was stirred in iPrOAc for a few hours, filtered and evaporated then lyophilized overnight.

A nucleophilic aromatic substitution (S_{N}Ar) then was performed:

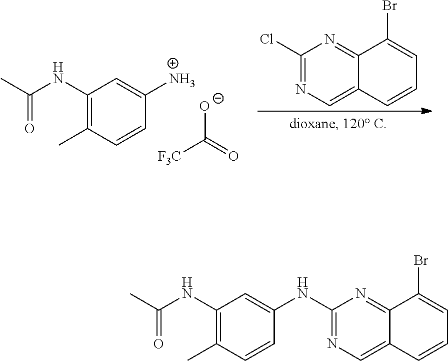 [SMILES: CC(=O)Nc1cc([NH3+])ccc1C ; O=C([O-])C(F)(F)F ; Clc1ncc2cccc(Br)c2n1 ; CC(=O)Nc1cc(Nc2ncc3cccc(Br)c3n2)ccc1C]

A 5.0 mL tapered microwave vial with triangular stir vane was charged with 3-acetamido-4-methylbenzenaminium 2,2,2-trifluoroacetate (91 mg, 0.33 mmol). Dioxane (1.64 mL) was added and the mixture stirred until significant dissolution was obtained. 8-bromo-2-chloro-quinazoline (160 mg, 0.66 mmol) was added. The vial was sealed and heated with stirring in an aluminum block (120° C.) for 1 hour. The reaction was evaporated to a dark residue. The residue was layered with IPA (40 mL) and brought to reflux with vigorous stirring, resulting in a dark, opaque mixture. Cooling to rt caused an apparent dark solid to precipitate. The mixture was stirred at room temperature overnight. The brown solid was recovered by filtration (87 mg).

A Suzuki coupling process then was performed:

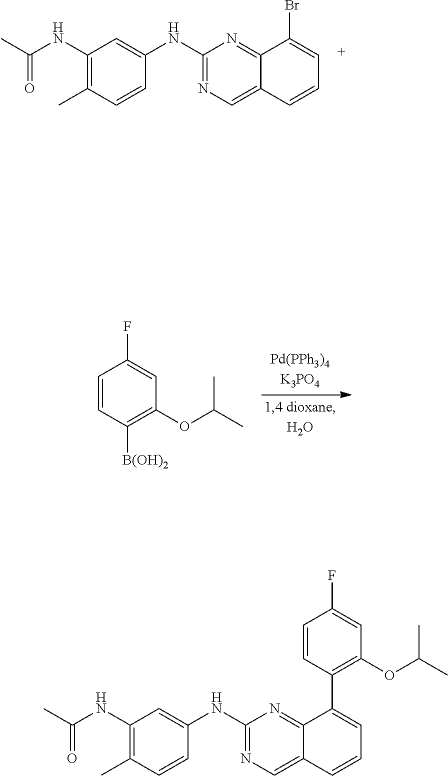 [SMILES: CC(=O)Nc1cc(Nc2ncc3cccc(Br)c3n2)ccc1C ; CC(C)Oc1cc(F)ccc1B(O)O ; CC(=O)Nc1cc(Nc2ncc3cccc(-c4ccc(F)cc4OC(C)C)c3n2)ccc1C]

A 0.5 mL-2.0 mL tapered microwave vial with triangular stir vane was charged with N-(5-((8-bromoquinazolin-2-yl)amino)-2-methylphenyl)acetamide (87 mg, 0.234 mmol, 1 equiv), (4-fluoro-2-isopropoxyphenyl)boronic acid (70 mg, 0.35 mmol, 1.5 equiv), K₃PO₄ (350 μL, 2M, 0.70 mmol, 3 equiv), and dioxane (1.27 mL, 0.185 M). Tetrakis(triphenylphosphine)palladium (54 mg, 0.05 mmol) was added, and the vial sealed, then heated in an aluminum block (105° C.) for 5 hours. The material was diluted in EtOAc and washed 3× with bicarb, following the addition of MgSO₄. The material was filtered and processed by roto evaporation. The crude material was purified by pTLC (40:1 CH₂Cl₂:MeOH) and one band was isolated containing pure product (30.5 mg, 29% yield).

### Example 63: Preparation of Cmpd50

An amidation process was performed:

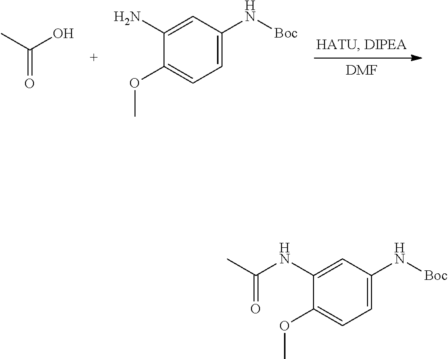

A 4 mL vial was equipped with tert-butyl (3-amino-4-methylphenyl)carbamate (150 mg, 0.63 mmol, 1 equiv), acetic acid (54 μL, 0.94 mmol, 1.5 equiv), HATU (263 mg, 0.69 mmol, 1.1 equiv) and DMF (4.2 mL, 0.15 M). Diisopropylethylamine was then added (329 μL, 1.88 mmol, 3 equiv) and the reaction was stirred at room temperature overnight. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was carried on to the next reaction.

A Boc removal process then was performed:

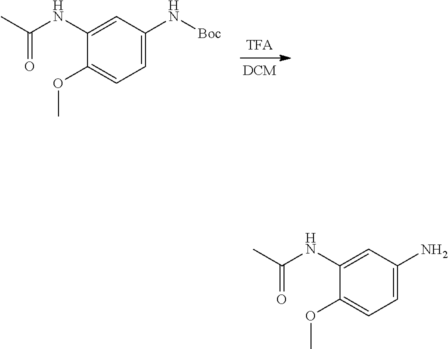 [SMILES: COc1ccc(NC(=O)OC(C)(C)C)cc1NC(C)=O ; COc1ccc(N)cc1NC(C)=O]

A solution of tert-butyl (3-acetamido-4-methoxyphenyl)carbamate (177 mg, 0.63 mmol) in DCM (5.04 mL) was treated with TFA (1.26 mL) at room temperature. The homogenous yellow reaction was stirred for 1 hr. Toluene (10 mL) was added, and the solution evaporated to a residue. The residue was diluted with EtOAc. HCl was then added and the layers were separated. The water layer was concentrated under reduced pressure and lyophilized overnight. The crude material (91 mg) was taken forward to the next reaction.

A nucleophilic aromatic substitution (S_{N}Ar) then was performed:

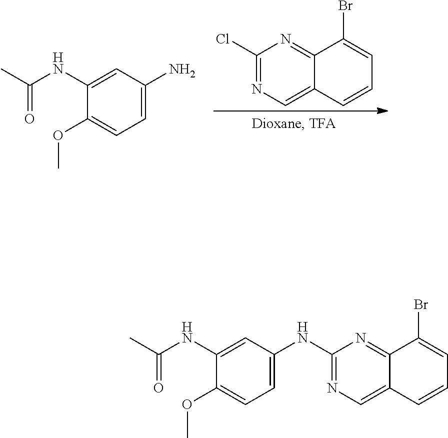 [SMILES: COc1ccc(N)cc1NC(C)=O ; Clc1ncc2cccc(Br)c2n1 ; COc1ccc(Nc2ncc3cccc(Br)c3n2)cc1NC(C)=O]

A 0.5-2.0 mL tapered microwave vial with triangular stir vane was charged with N-(5-amino-2-methoxyphenyl)acetamide (91 mg, 0.5 mmol). Dioxane (2.28 mL) was added, and the mixture stirred until significant dissolution was obtained. 8-bromo-2-chloro-quinazoline (246 mg, 1.01 mmol) was added. AcOH (0.25 mL) was added. The vial was sealed and heated with stirring in an aluminum block (120° C.) for 1 hour. LCMS showed the reaction was complete. The reaction was diluted with 2 mL of dioxane and heated for a few minutes. The residue was cooled to rt causing a solid to precipitate. The yellow solid was recovered by filtration and purified by pTLC using 40:1 DCM:MeOH, rf: 0.6. Pure product was obtained (40.5 mg, 21% yield).

A Suzuki coupling process then was performed:

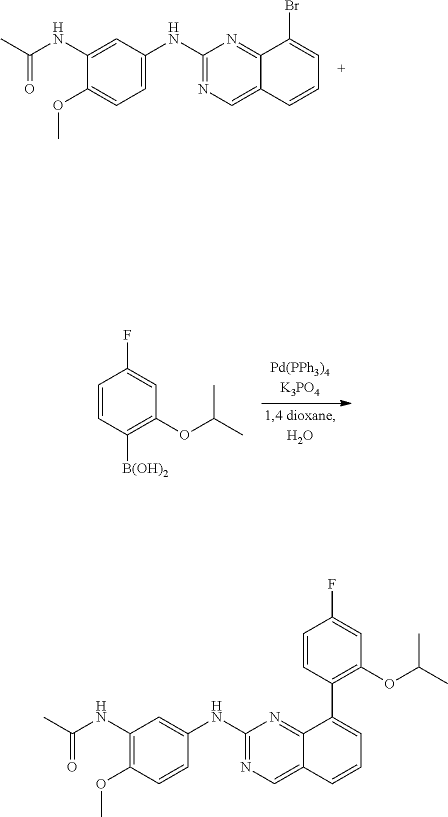 [SMILES: COc1ccc(Nc2ncc3cccc(Br)c3n2)cc1NC(C)=O ; CC(C)Oc1cc(F)ccc1B(O)O ; COc1ccc(Nc2ncc3cccc(-c4ccc(F)cc4OC(C)C)c3n2)cc1NC(C)=O]

A 0.5 mL-2.0 mL tapered microwave vial with triangular stir vane was charged with N-(5-((8-bromoquinazolin-2-yl)amino)-2-methoxyphenyl)acetamide (40.5 mg, 0.1 mmol, 1 equiv), (4-fluoro-2-isopropoxyphenyl)boronic acid (31 mg, 0.16 mmol, 1.5 equiv), K₃PO₄ (157 μL, 2M, 0.3 mmol, 3 equiv), and dioxane (0.56 mL, 0.185 M). Tetrakis(triphenylphosphine)palladium (23 mg, 0.02 mmol) was added, and the vial sealed, then heated in an aluminum block (105° C.) for 5 hours. The reaction was diluted with excess EtOAc and dried over MgSO₄. The crude material was purified by pTLC (40:1 CH₂Cl₂:MeOH). One band was isolated containing pure product (39.5 mg, 82% yield).

### Example 64: Preparation of Cmpd60

Cmpd51 described in Example 24 was utilized as a reactant for the preparation of Cmpd60 here, and for the preparation of compounds described in Examples 65-75. An amidation process was performed:

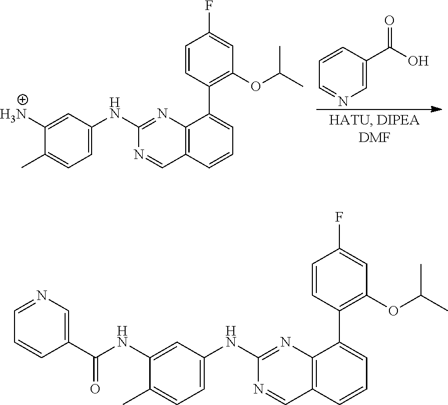 [SMILES: Cc1ccc(Nc2ncc3cccc(-c4ccc(F)cc4OC(C)C)c3n2)cc1[NH3+] ; O=C(O)c1cccnc1 ; Cc1ccc(Nc2ncc3cccc(-c4ccc(F)cc4OC(C)C)c3n2)cc1NC(=O)c1cccnc1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), Nicotinic acid (6.9 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (5.7 mg, 30% yield).

### Example 65: Preparation of Cmpd61

An amidation process was performed:

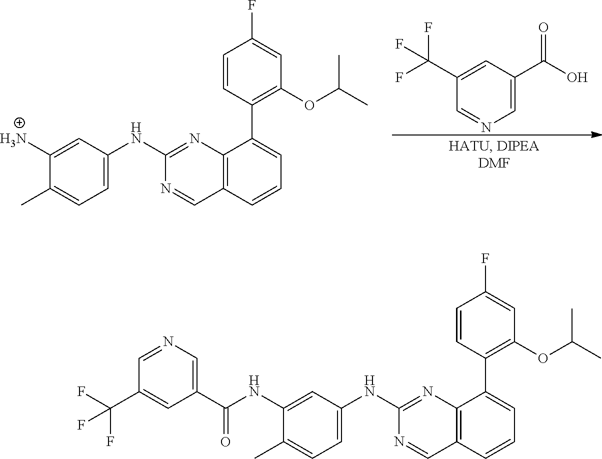 [SMILES: Cc1ccc(Nc2ncc3cccc(-c4ccc(F)cc4OC(C)C)c3n2)cc1[NH3+] ; O=C(O)c1cncc(C(F)(F)F)c1 ; Cc1ccc(Nc2ncc3cccc(-c4ccc(F)cc4OC(C)C)c3n2)cc1NC(=O)c1cncc(C(F)(F)F)c1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), 5-trifluoromethyl-nicotinic acid (10.7 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (5.6 mg, 26% yield).

### Example 66: Preparation of Cmpd62

An amidation process was performed:

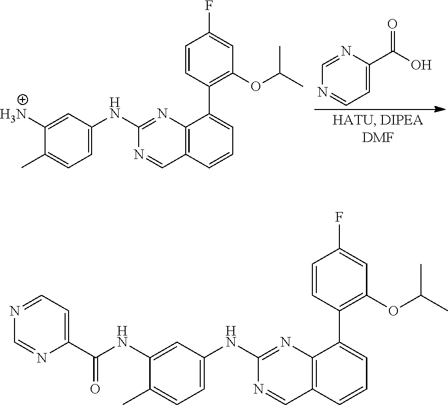 [SMILES: Cc1ccc(Nc2ncc3cccc(-c4ccc(F)cc4OC(C)C)c3n2)cc1[NH3+] ; O=C(O)c1ccncn1 ; Cc1ccc(Nc2ncc3cccc(-c4ccc(F)cc4OC(C)C)c3n2)cc1NC(=O)c1ccncn1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), pyrimidine-4-carboxylic acid (6.9 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (8.5 mg, 45% yield).

### Example 67: Preparation of Cmpd63

An amidation process was performed:

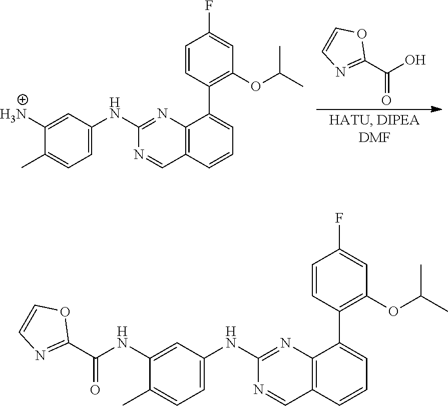 [SMILES: Cc1ccc(Nc2ncc3cccc(-c4ccc(F)cc4OC(C)C)c3n2)cc1[NH3+] ; O=C(O)c1ncco1 ; Cc1ccc(Nc2ncc3cccc(-c4ccc(F)cc4OC(C)C)c3n2)cc1NC(=O)c1ncco1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), oxazole-2-carboxylic acid (6.3 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (5.9 mg, 32% yield).

### Example 68: Preparation of Cmpd68

An amidation process was performed:

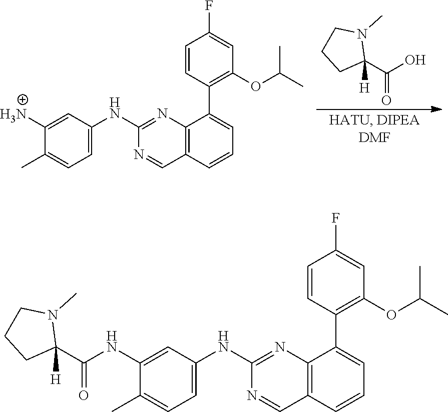

A 4 mL vial was charged with 5-((8-(4-fluoro-2-isopropoxyphenyl)quinazolin-2-yl)amino)-2-methylbenzenaminium (15 mg, 0.037 mmol, 1 equiv), methyl-L-proline (7.2 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (3.9 mg, 20% yield).

### Example 69: Preparation of Cmpd69

An amidation process was performed:

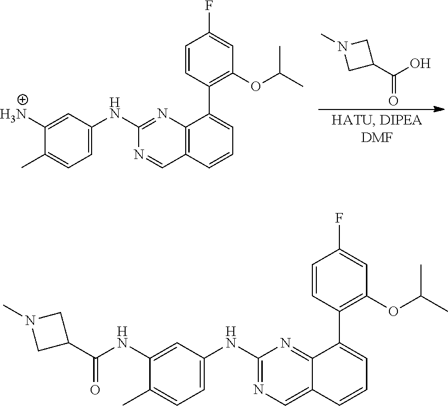

A 4 mL vial was charged with 5-((8-(4-fluoro-2-isopropoxyphenyl)quinazolin-2-yl)amino)-2-methylbenzenaminium (15 mg, 0.037 mmol, 1 equiv), 1-methylazetidine-3-carboxylic acid (6.4 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (7.3 mg, 39% yield).

### Example 70: Preparation of Cmpd70

An amidation process was performed:

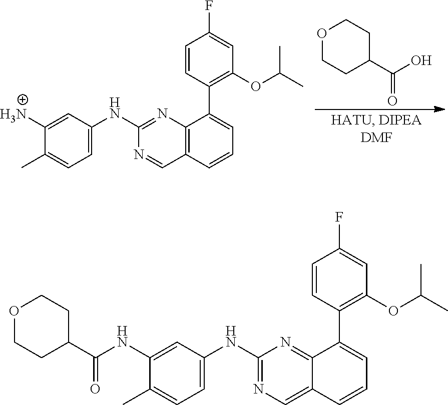 [SMILES: Cc1ccc(Nc2ncc3cccc(-c4ccc(F)cc4OC(C)C)c3n2)cc1[NH3+] ; O=C(O)C1CCOCC1 ; Cc1ccc(Nc2ncc3cccc(-c4ccc(F)cc4OC(C)C)c3n2)cc1NC(=O)C1CCOCC1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), tetrahydro-2H-pyran-4-carboxylic acid (7.3 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (9.9 mg, 52% yield).

### Example 71: Preparation of Cmpd71

An amidation process was performed:

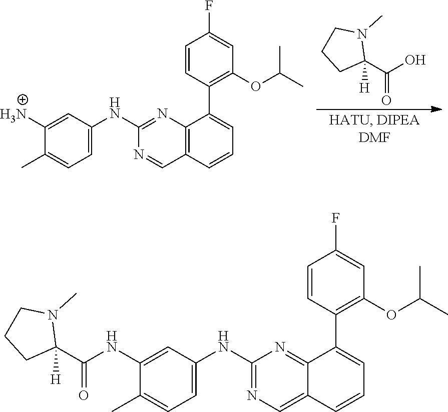

A 4 mL vial was charged with 5-((8-(4-fluoro-2-isopropoxyphenyl)quinazolin-2-yl)amino)-2-methylbenzenaminium (15 mg, 0.037 mmol, 1 equiv), methyl-D-proline (7.2 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (3.8 mg, 20% yield).

### Example 72: Preparation of Cmpd72

An amidation process was performed:

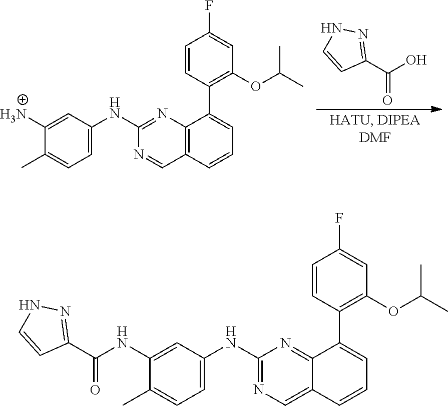 [SMILES: Cc1ccc(Nc2ncc3cccc(-c4ccc(F)cc4OC(C)C)c3n2)cc1[NH3+] ; O=C(O)c1cc[nH]n1 ; Cc1ccc(Nc2ncc3cccc(-c4ccc(F)cc4OC(C)C)c3n2)cc1NC(=O)c1cc[nH]n1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), 1H-pyrazole-3-carboxylic acid (10.6 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. After overnight reaction, the reaction was monitored by LCMS. The reaction was incomplete. Additional reagents were added and the reaction was stirred for 2 hr at 40° C. The reaction still was incomplete. The reaction was heated to 60° C. and stirred for 2 hr. The reaction was diluted with sat. aq. NaHCO₃ and EtOAC. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (4.6 mg, 25% yield).

### Example 73: Preparation of Cmpd73

An amidation process was performed:

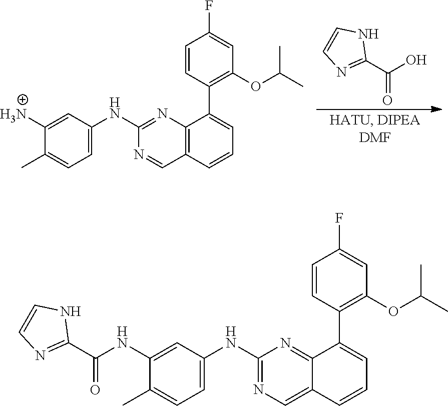 [SMILES: Cc1ccc(Nc2ncc3cccc(-c4ccc(F)cc4OC(C)C)c3n2)cc1[NH3+] ; O=C(O)c1ncc[nH]1 ; Cc1ccc(Nc2ncc3cccc(-c4ccc(F)cc4OC(C)C)c3n2)cc1NC(=O)c1ncc[nH]1]

A 4 mL vial was charged with 5-((8-(4-fluoro-2-isopropoxyphenyl)quinazolin-2-yl)amino)-2-methylbenzenaminium (15 mg, 0.037 mmol, 1 equiv), 1H-imidazole-2-carboxylic acid (6.3 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature overnight. The reaction was incomplete.

Additional reagents were added, and the reaction was stirred for 2 hr at 40° C. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford product (3.9 mg, 21% yield).

### Example 74: Preparation of Cmpd74

An amidation process was performed:

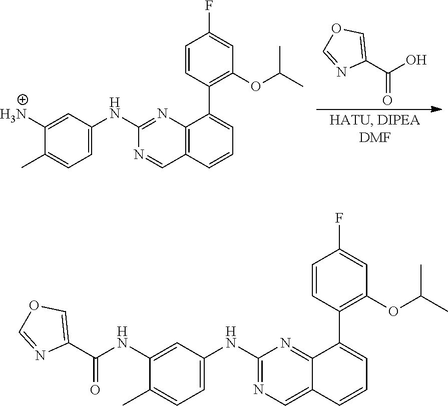 [SMILES: Cc1ccc(Nc2ncc3cccc(-c4ccc(F)cc4OC(C)C)c3n2)cc1[NH3+] ; O=C(O)c1cocn1 ; Cc1ccc(Nc2ncc3cccc(-c4ccc(F)cc4OC(C)C)c3n2)cc1NC(=O)c1cocn1]

A 4 mL vial was equipped with 5-((8-(4-fluoro-2-isopropoxyphenyl)quinazolin-2-yl)amino)-2-methylbenzenaminium (15 mg, 0.037 mmol, 1 equiv), oxazole-4-carboxylic acid (6.3 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford pure product (5.9 mg, 32% yield).

### Example 75: Preparation of Cmpd65

An amidation process was performed:

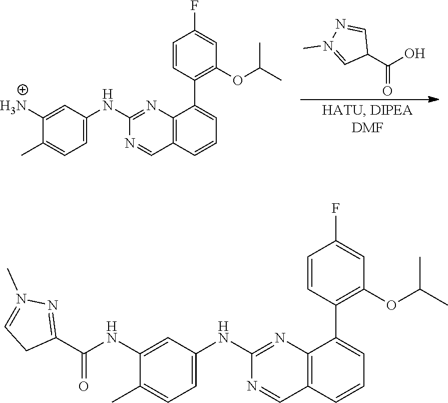

A 4 mL vial was charged with 5-((8-(4-fluoro-2-isopropoxyphenyl)quinazolin-2-yl)amino)-2-methylbenzenaminium (15 mg, 0.037 mmol, 1 equiv), 1-methyl-4H-114-pyrazole-3-carboxylic acid (7.1 mg, 0.056 mmol, 1.5 equiv), HATU (15.5 mg, 0.041 mmol, 1.1 equiv) and DMF (750 μL, 0.05 M). Diisopropylethylamine was then added (19.4 μL, 0.112 mmol, 3 equiv) and the reaction was stirred at room temperature. The reaction was diluted with sat. aq. NaHCO₃ and EtOAc. The aqueous phase was extracted with EtOAc (3×). The combined organic layer was washed with water to remove any remaining DMF, then dried over MgSO₄, and concentrated under reduced pressure. The crude material was purified by pTLC (40:1 DCM:MeOH) to afford product (4.2 mg, 22% yield).

### Example 76: Preparation of Cmpd57

An amidation process was performed:

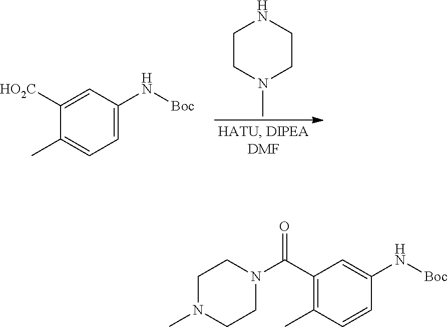

A 20 mL screw top scintillation with magnetic stir bar was charged with 5-(tert-butoxycarbonylamino)-2-methylbenzoic acid (296 mg, 1.178 mmol). DMF (2 mL) was added and the mixture stirred until dissolution was complete, affording a yellow/orange solution. A solution of 1-methylpiperazine (177 mg, 1.767 mmol) in DMF (2.5 mL) was added. DIEA (310 μL, 1.767 mmol) was added followed by HATU (672 mg, 1.767 mmol). The yellow solution was stirred at room temperature for 4 hr. LCMS showed the reaction was complete. The reaction was diluted with iPrOAc (40 mL) and washed with aqueous bicarbonate (3× wash). The organic phase was dried over MgSO₄, filtered and evaporated to a residue. The material was carried forward into a deprotection process without quantitation.

A Boc removal process then was performed:

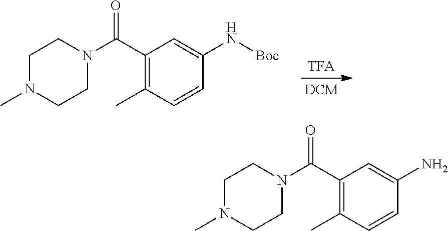 [SMILES: Cc1ccc(NC(=O)OC(C)(C)C)cc1C(=O)N1CCN(C)CC1 ; Cc1ccc(N)cc1C(=O)N1CCN(C)CC1]

A 250 mL round bottom flask containing tert-butyl N-[4-methyl-3-(4-methylpiperazine-1-carbonyl)phenyl]carbamate was treated with DCM (30 mL). The yellow solution was treated with TFA (6 mL) and stirred at room temperature for 1 hr. LCMS showed the reaction was complete. Toluene (20 mL) was added and the solution evaporated to a residue. iPrOAc (25 mL) was added and the solution stirred at room temperature over the weekend. No precipitation was observed. The solution was recovered and evaporated to an orange/brown residue (1.900 g).

A nucleophilic aromatic substitution (S_{N}Ar) was performed:

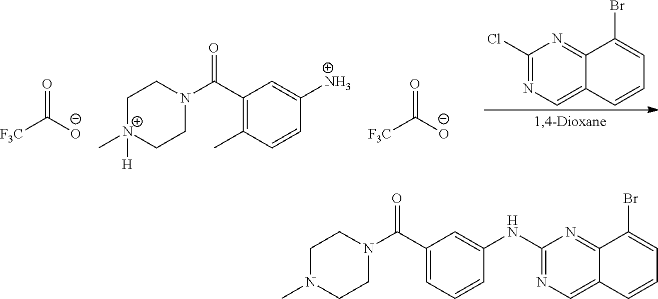 [SMILES: O=C([O-])C(F)(F)F ; Cc1ccc([NH3+])cc1C(=O)N1CC[NH+](C)CC1 ; O=C([O-])C(F)(F)F ; Clc1ncc2cccc(Br)c2n1 ; CN1CCN(C(=O)c2cccc(Nc3ncc4cccc(Br)c4n3)c2)CC1]

[4-methyl-3-(4-methylpiperazin-4-ium-1-carbonyl)phenyl]ammonium;2,2,2-trifluoroacetate (1.900 g, 4.118 mmol) was partially dissolved and partially suspended in dioxane (8 mL). Not all the material dissolved. Most of the oily salt was transferred into two 2.0-5.0 mL microwave vials with stir bar. Each vial was treated with 8-bromo-2-chloro-quinazoline (1.003 g, 8.237 mmol in total). The vials were sealed and heated in an aluminum block (125° C.) for 1 hr. LCMS showed the reaction was complete. The combined solids were recovered by filtration using enough dioxane to rinse them into the filter funnel. The cream-colored solid was allowed to stand under lyophilizer vacuum overnight (919 mg). The solid and mother liquor were sampled for LCMS. The mother liquor had deposited a solid in the filtration flask that did not redissolve even when heating 100 mg of the solid with EtOAc in a 40 mL screw top vial. Excess iPrOAc and 1N HCl ere added and the two-phase mixture vigorously stirred overnight. Stirring was stopped and the mixture separated into a lower, turbid, cream-colored layer and a clear, upper organic layer. There was some detergent-like emulsification. The organic layer was drawn off by pipette and the operation repeated twice more. The aqueous phase became visibly less turbid. The aqueous phase was sampled for LCMS. The desired product was present with improved purity.

The remaining 819 mg of solid was treated with a 1:1 mixture of iPrOAc and 1N HCl (100 mL each) and vigorously stirred overnight. The mixture was transferred to a 500 mL separatory funnel. Most of very pale green turbid aqueous phase was drained out and the turbid, colorless organic phase washed with 1N HCl until a clean phase separation could be observed (with washing). The organic phase was set aside and the aqueous phase (about 600 mL) was put into a clean round bottom flask, vigorously stirred and neutralized to pH 8 with solid bicarbonate. The now visibly more turbid aqueous phase was stirred with iPrOAc (300 mL), then transferred to the separatory funnel in portions. The clear aqueous phase was drained out and the pale green turbid organic phase washed with saturated bicarbonate until a clean phase separation was obtained. The combined organic phase was dried over MgSO₄ and filtered, affording a clear pale green solution. The solution was evaporated to a white semi-solid (55 mg). The first mother liquor (iPrOAc) was decanted, leaving a white solid which is sparingly soluble in MeOH. LCMS showed it has the desired product. The solid was suspended in EtOAc and vigorously stirred with aqueous bicarbonate for 1 hour. The layers were separated with a small amount of emulsion collected separately. The organic phase was dried over MgSO₄, filtered and evaporated to a residue. LCMS showed the desired product was a major component. The residue was combined with the previously-isolated material to yield an orange oil (170 mg).

The oil was dissolved in acetone (2 mL) and applied to a 30 gram C18 column. The column was eluted as shown (10% to 100% AN/water over 20 column volumes). After LCMS analysis the fractions that did not contain any recovered quinazoline compound were combined and the organics removed along with most of the water, leaving a wet yellow semisolid. The mixture was diluted with excess EtOAc and dried over MgSO₄, filtered and evaporated to a second yellow semi-solid (40 mg). This first isolated material was taken forward into a Suzuki reaction. The mixed fractions were recovered (23 mg).

A Suzuki coupling process then was performed:

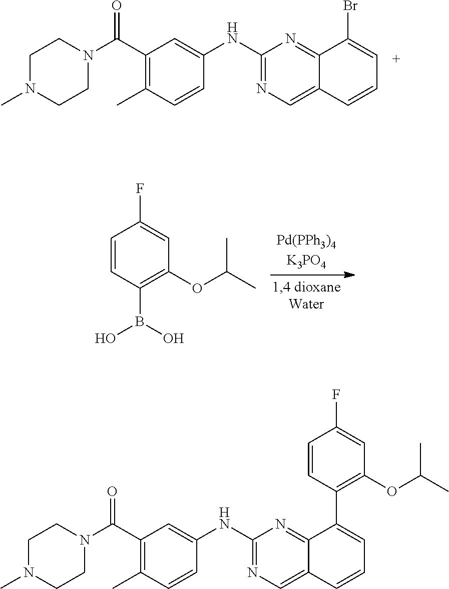 [SMILES: Cc1ccc(Nc2ncc3cccc(Br)c3n2)cc1C(=O)N1CCN(C)CC1 ; CC(C)Oc1cc(F)ccc1B(O)O ; Cc1ccc(Nc2ncc3cccc(-c4ccc(F)cc4OC(C)C)c3n2)cc1C(=O)N1CCN(C)CC1]

A 0.5-2.0 mL tapered microwave vial with a triangular stir vane was charged with a solution of [5-[(8-bromoquinazolin-2-yl)amino]-2-methyl-phenyl]-(4-methylpiperazin-1-yl)methanone (36 mg, 0.082 mmol) in dioxane (1 mL). Aqueous K₃PO₄ (2M, 120 μL, 0.245 mmol) was added, followed by (4-fluoro-2-isopropoxyphenyl) boronic acid (24 mg, 0.123 mmol). Tetrakistriphenylphosphine palladium (19 mg, 0.016 mmol) was added, the vial sealed and heated in an aluminum block (105° C.) for 4 hr. LCMS showed the reaction was done. The black mixture was diluted with excess EtoAc and dried over MgSO₄. Filtration and evaporation afforded an orange/brown residue (84 mg). The material was applied onto a 1000 μm pTLC plate. The plate was eluted with 20:1 DCM/MeOH. The indicated band was collected as an orange/brown oil (9 mg).

### Example 77: Preparation of Cmpd20

An amidation process was performed:

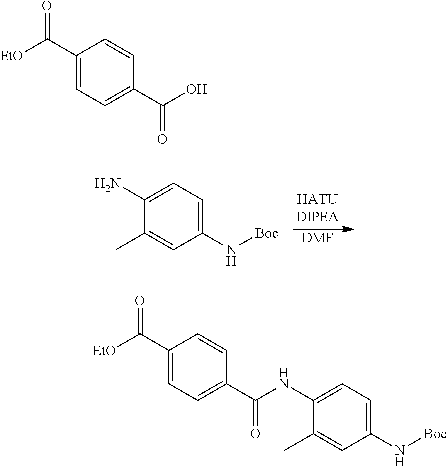

A 40 mL screw top vial with stir bar was charged with a solution of tert-butyl N-(4-amino-3-methylphenyl) carbamate (500 mg, 2.249 mmol) in DMF (8 mL). The dark solution was treated with 4-ethoxycarbonylbenzoic acid (655 mg, 3.374 mmol), then DIEA (590 μL, 3.375 mmol), then HATU (1.282 g, 3.372 mmol). The dark solution was stirred at room temperature for 16 hours. LCMS showed the reaction was well advanced. The reaction was poured into excess iPrOAc and washed with aqueous bicarbonate (3× wash). The dark brown organic phase was dried over MgSO₄, filtered and evaporated to a brown solid. The material was used without quantification or further purification.

A Boc removal process was performed:

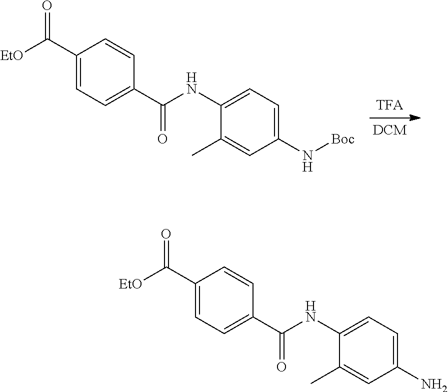 [SMILES: CCOC(=O)c1ccc(C(=O)Nc2ccc(NC(=O)OC(C)(C)C)cc2C)cc1 ; CCOC(=O)c1ccc(C(=O)Nc2ccc(N)cc2C)cc1]

The crude product from was dissolved in DCM (30 mL) and treated with TFA (6 mL). The dark brown solution was stirred at room temperature overnight. LCMS showed the reaction was complete (there was no starting material mass present in the peak that remained at 4.78). The dark reaction was diluted with toluene (10 mL) and evaporated to a residue. The residue was treated with iPrOAc (30 mL), causing the formation of a powdery solid. The mixture was stirred for 30 min. The solid was recovered by filtration. LCMS showed a double peak and both peaks corresponded to the product. The filter funnel was allowed to stand under lyophilizer vacuum overnight, yielding a pale magenta colored powder (778 mg, 84% overall from tert-butyl N-(4-amino-3-methyl-phenyl)carbamate).

A nucleophilic aromatic substitution (S_{N}Ar) then was performed:

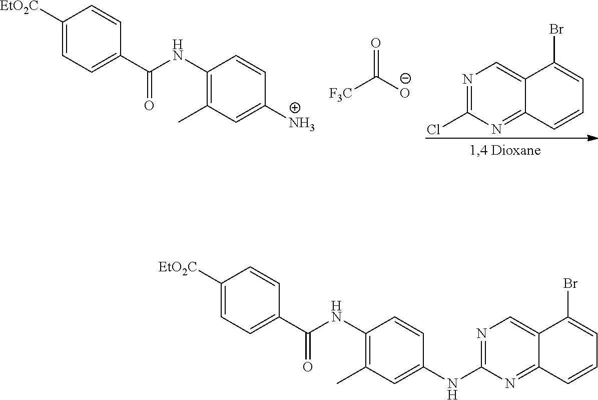 [SMILES: CCOC(=O)c1ccc(C(=O)Nc2ccc([NH3+])cc2C)cc1 ; O=C([O-])C(F)(F)F ; Clc1ncc2c(Br)cccc2n1 ; CCOC(=O)c1ccc(C(=O)Nc2ccc(Nc3ncc4c(Br)cccc4n3)cc2C)cc1]

A 2.0-5.0 mL microwave vial with stir bar was charged with [4-[(4-ethoxycarbonylbenzoyl)amino]-3-methylphenyl]ammonium;2,2,2-trifluoroacetate (388 mg, 0.941 mmol). 5-bromo-2-chloro-quinazoline (458 mg, 1.882 mmol) was added, followed by dioxane (3.5 mL). The vial was sealed and the orange suspension was heated in an aluminum block (125° C.) for 1 hour. The orange suspension was diluted with dioxane (5 mL) and filtered, affording an orange/brown solid and a brown mother liquor. The solid was allowed to stand under lyophilizer vacuum overnight, yielding an orange solid (573 mg) that was overweight. A portion of the crude mixture was chromatographed on a 1000 micrometer pTLC plate to provide material for hydrolysis and submission. Some of the rest (340 mg) was taken forward into a Suzuki reaction (as described in Example 78, for example).

An ester hydrolysis was performed:

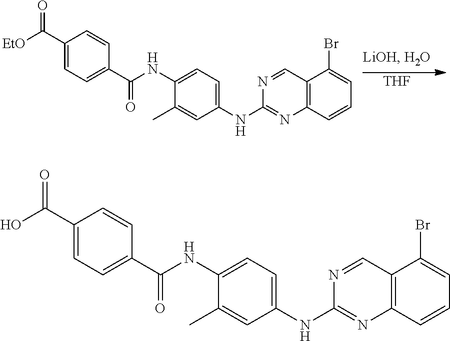 [SMILES: CCOC(=O)c1ccc(C(=O)Nc2ccc(Nc3ncc4c(Br)cccc4n3)cc2C)cc1 ; Cc1cc(Nc2ncc3c(Br)cccc3n2)ccc1NC(=O)c1ccc(C(=O)O)cc1 ; "THF"]

A 0.5-2.0 mL microwave vial with stir bar was charged with a solution of ethyl 4-[[4-[(5-bromoquinazolin-2-yl)amino]-2-methyl-phenyl]carbamoyl]benzoate (11 mg, 0.022 mmol) in THF (1 mL). Water (20 μL) was added followed by LiOH hydrate (9 mg, 0.218 mmol). The vial was sealed and heated in an aluminum block (85° C.) for 24 hr. The yellow mixture was treated with 1N HCl (2 mL) and recovered into a tared 20 mL scintillation vial. EtOAc and 1N HCl were used to rinse the reaction vial. The 2-phase mixture was evaporated free of organics, leaving a bright yellow solid floating in a clear, colorless aqueous phase. The mixture was clamped in the hood at an angle and the solid allowed to settle to the bottom of the vial overnight. The aqueous phase was removed by syringe and the solid rinsed twice more with water. The remaining solid was allowed to stand under lyophilizer vacuum overnight, affording a bright yellow solid (9 mg).

### Example 78: Preparation of Cmpd26

The same amidation and Boc removal processes described in the first two steps for preparing Cmpd20 in Example 77 were performed, affording [4-[(4-ethoxycarbonylbenzoyl)amino]-3-methylphenyl]ammonium. A nucleophilic aromatic substitution (S_{N}Ar) then was performed:

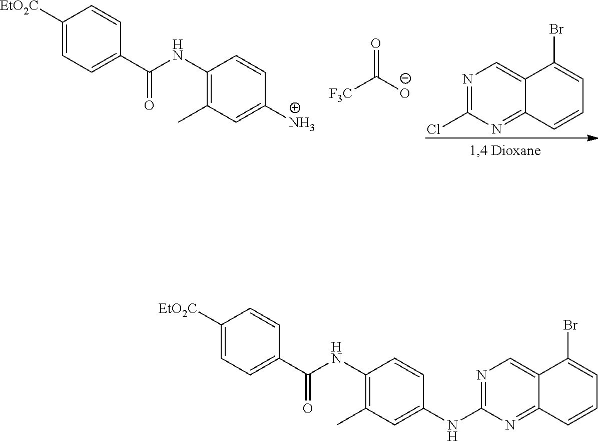 [SMILES: CCOC(=O)c1ccc(C(=O)Nc2ccc([NH3+])cc2C)cc1 ; O=C([O-])C(F)(F)F ; Clc1ncc2c(Br)cccc2n1 ; CCOC(=O)c1ccc(C(=O)Nc2ccc(Nc3ncc4c(Br)cccc4n3)cc2C)cc1]

A 2.0-5.0 mL microwave vial with stir bar was charged with [4-[(4 ethoxycarbonylbenzoyl)amino]-3-methylphenyl]ammonium;2,2,2-trifluoroacetate (388 mg, 0.941 mmol). 8-bromo-2-chloro-quinazoline (458 mg, 1.882 mmol) was added, followed by dioxane (5 mL). The vial was sealed, and the orange suspension heated in an aluminum block (125° C.) for 1 hour. The orange suspension was diluted with more dioxane (5 mL) and filtered, affording an orange solid and a brown mother liquor. The solid was allowed to stand under lyophilizer vacuum overnight, yielding an orange solid (349 mg).

A Suzuki coupling process then was performed:

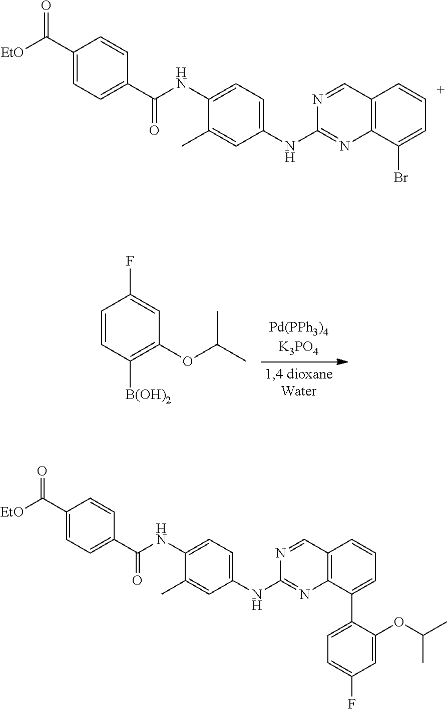 [SMILES: CCOC(=O)c1ccc(C(=O)Nc2ccc(Nc3ncc4cccc(Br)c4n3)cc2C)cc1 ; CC(C)Oc1cc(F)ccc1B(O)O ; CCOC(=O)c1ccc(C(=O)Nc2ccc(Nc3ncc4cccc(-c5ccc(F)cc5OC(C)C)c4n3)cc2C)cc1]

A 2.0-5.0 mL microwave vial with stir bar was charged with ethyl 4-[[4-[(8-bromoquinazolin-2-yl)amino]-2-methylphenyl]carbamoyl]benzoate (340 mg, 0.673 mmol), followed by dioxane (4 mL). The mixture was stirred for 5 min to effect suspension of the solid. Aqueous K3P04 (2M, 1 mL, 2.000 mmol) was added. (4-fluoro-2-isopropoxyphenyl) boronic acid (200 mg, 1.009 mmol) was added, then tetrakistriphenylphosphinepalladium (155 mg, 0.135 mmol). The vial was sealed and heated in an aluminum block (125° C.) for 4 hr. LCMS showed the reaction was complete. The mixture was diluted with excess iPrOAc, dried over MgSO₄, filtered and evaporated to a black residue. The residue was layered with IPA (15 mL), brought to a boil with stirring, then allowed to cool to rt and stirred for 2 hr. The precipitated black solid was recovered by filtration (45 mg). The mother liquor was recovered and evaporated to an orange semi-solid (401 mg).

An ester hydrolysis then was performed:

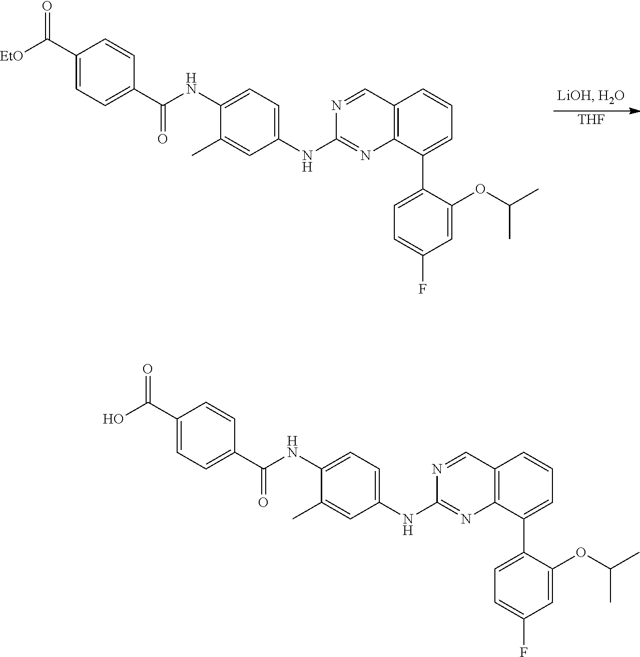 [SMILES: CCOC(=O)c1ccc(C(=O)Nc2ccc(Nc3ncc4cccc(-c5ccc(F)cc5OC(C)C)c4n3)cc2C)cc1 ; Cc1cc(Nc2ncc3cccc(-c4ccc(F)cc4OC(C)C)c3n2)ccc1NC(=O)c1ccc(C(=O)O)cc1 ; "THF"]

A 0.5-2.0 mL tapered microwave vial with triangular stir vane was charged with ethyl 4-[[4-[[8-(4-fluoro-2-isopropoxy-phenyl)quinazolin-2-yl]amino]-2-methyl-phenyl]carbamoyl]benzoate (45 mg, 0.078 mmol). THF (1 mL) was added, followed by water (20 μL) and LiOH hydrate (33 mg, 0.778 mmol). The vial was sealed, stirred and heated in an aluminum block (85° C.) for 24 hr. The black mixture was treated with 1N HCl (2 mL) and THF (1 mL). The mixture was vigorously stirred, then transferred to a 20 mL scintillation vial with THF and 1N HCl rinsing. The THF was evaporated, leaving a dark grey solid suspended in water. The solid was recovered by filtration and the filter funnel with the solid was allowed to stand under lyophilizer vacuum for 24 hr, affording isolated product (41 mg).

### Example 79: Preparation of Cmpd27

An amidation process was performed:

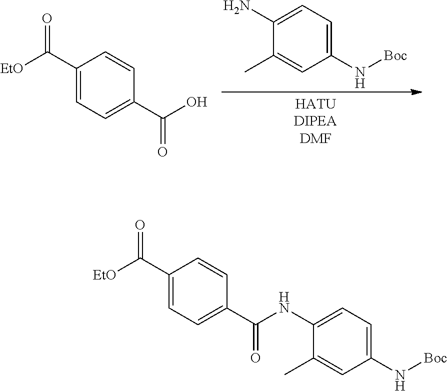 [SMILES: CCOC(=O)c1ccc(C(=O)O)cc1 ; Cc1cc(NC(=O)OC(C)(C)C)ccc1N ; CCOC(=O)c1ccc(C(=O)Nc2ccc(NC(=O)OC(C)(C)C)cc2C)cc1]

A 40 mL screw top vial with stir bar was charged with a solution of tert-butyl N-(4-amino-3-methylphenyl) carbamate (500 mg, 2.249 mmol) in DMF (8 mL). The dark solution was treated with 4-ethoxycarbonylbenzoic acid (655 mg, 3.374 mmol), then DIEA (590 μL, 3.375 mmol), then HATU (1.282 g, 3.372 mmol). The dark solution was stirred at room temperature for 16 hours. LCMS showed the reaction was well advanced. The reaction was poured into excess iPrOAc and washed with aqueous bicarbonate (3× wash). The dark brown organic phase was dried over MgSO₄, filtered and evaporated to a brown solid. The material was used without quantification or further purification.

A Boc removal process then was performed:

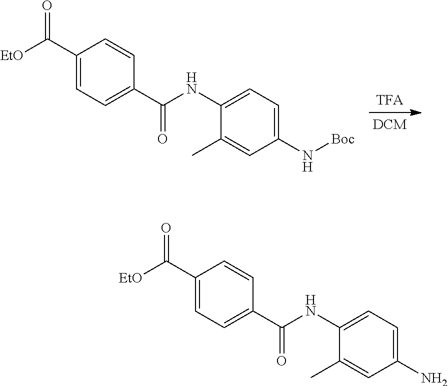 [SMILES: CCOC(=O)c1ccc(C(=O)Nc2ccc(NC(=O)OC(C)(C)C)cc2C)cc1 ; CCOC(=O)c1ccc(C(=O)Nc2ccc(N)cc2C)cc1]

The crude product was dissolved in DCM (30 mL) and treated with TFA (6 mL). The dark brown solution was stirred at room temperature overnight. LCMS showed the reaction was complete (there was no starting material mass present in the peak that remained at 4.78). The dark reaction was diluted with toluene (10 mL) and evaporated to a residue. The residue was treated with iPrOAc (30 mL), causing the formation of a powdery solid. The mixture was stirred for 30 min. The solid was recovered by filtration. LCMS showed a double peak but the two peaks corresponded to the product. The filter funnel was allowed to stand under lyophilizer vacuum overnight, affording a pale magenta colored powder (778 mg, 84% overall from tert-butyl N-(4-amino-3-methyl-phenyl)carbamate).

A nucleophilic aromatic substitution (S_{N}Ar) then was performed:

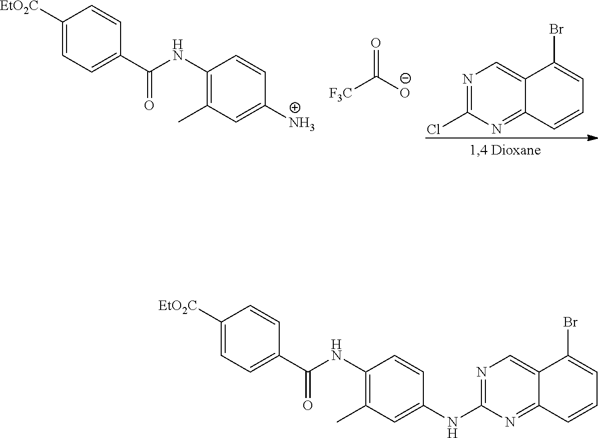 [SMILES: CCOC(=O)c1ccc(C(=O)Nc2ccc([NH3+])cc2C)cc1 ; O=C([O-])C(F)(F)F ; Clc1ncc2c(Br)cccc2n1 ; CCOC(=O)c1ccc(C(=O)Nc2ccc(Nc3ncc4c(Br)cccc4n3)cc2C)cc1]

A 2.0-5.0 mL microwave vial with stir bar was charged with [4-[(4-ethoxycarbonylbenzoyl)amino]-3-methylphenyl]ammonium;2,2,2-trifluoroacetate (388 mg, 0.941 mmol). 5-bromo-2-chloro-quinazoline (458 mg, 1.882 mmol) was added, followed by dioxane (3.5 mL). The vial was sealed and the orange suspension was heated in an aluminum block (125° C.) for 1 hour. The orange suspension was diluted with dioxane (5 mL) and filtered, affording an orange/brown solid and a brown mother liquor. The solid was allowed to stand under lyophilizer vacuum overnight, yielding an orange solid (573 mg), which was overweight. A portion of the crude was chromatographed on a 1000 micrometer pTLC plate to provide material for hydrolysis and submission. Some of the rest (340 mg) was taken forward into a Suzuki reaction.

A Suzuki coupling process then was performed:

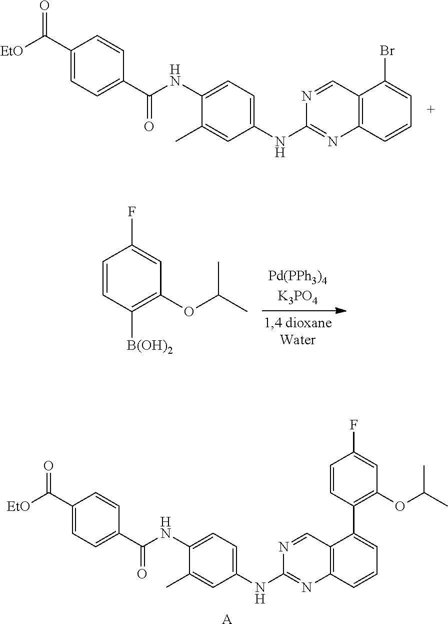

A 2.0-5.0 mL microwave vial with stir bar was charged with ethyl 4-[[4-[(5-bromoquinazolin-2-yl)amino]-2-methylphenyl]carbamoyl]benzoate (340 mg, 0.673 mmol), followed by dioxane (4 mL). The mixture was stirred for 5 min to effect suspension of the solid. Aqueous K3P04 (2M, 1 mL, 2.000 mmol) was added. (4-fluoro-2-isopropoxyphenyl) boronic acid (200 mg, 1.009 mmol) was added, then tetrakistriphenylphosphinepalladium (155 mg, 0.135 mmol). The vial was sealed and heated in an aluminum block (125° C.) for 4 hr. LCMS showed the reaction was complete. The mixture was diluted with excess iPrOAc, dried over MgSO₄, filtered and evaporated to an orange/brown residue (534 mg). TLC (20:1 DCM/MeOH) showed the reaction was complete. The residue was loaded onto a silica gel column and chromatographed on a DCM/MeOH gradient (0% to 20% MeOH/DCM over 16 column volumes, then 20% MeOH/DCM for 7 column volumes. LCMS showed the desired compound was contained in the first major peak. Two pure fractions (9 and 10) were collected, affording a yellow/orange residue (215 mg).

An ester hydrolysis then was performed:

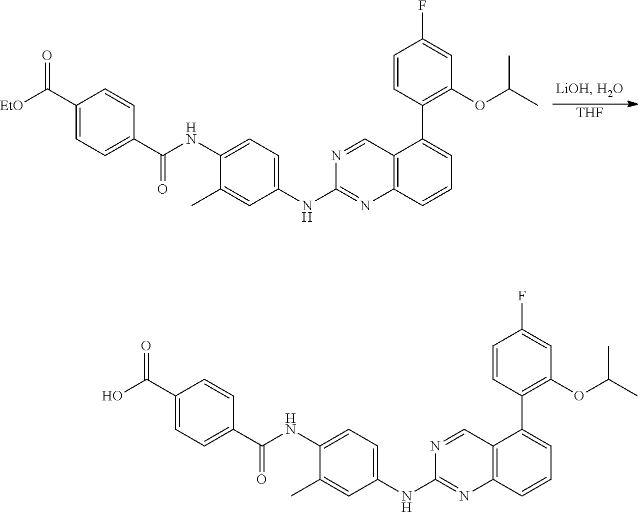

A 2.0-5.0 mL microwave vial was charged with ethyl 4-[[4-[[5-(4-fluoro-2-isopropoxy-phenyl)quinazolin-2-yl]amino]-2-methyl-phenyl]carbamoyl]benzoate (210 mg, 0.363 mmol) as a solution in THF (3 mL). Water (65 μL, 3.630 mmol) was added followed by LiOH hydrate (152 mg, 3.629 mmol). The reaction was heated in an aluminum block (85° C.) for 24 hr. The mixture was treated with 1N HCl and EtOAc. The two-phase mixture was stirred at room temperature in a 40 mL screw top vial until all solids had dissolved, leaving a very pale yellow aqueous and an orange/brown organic. The mixture was transferred in portions to another 40 mL screw to vial and evaporated free of organic, leaving an orange/brown solid floating in a clear aqueous. The mixture was momentarily brought to boiling with vigorous stirring and then allowed to cool and stir for 3 hr, leaving a finely divided orange solid. The solid was recovered by filtration and allowed to stand under lyophilizer vacuum overnight, affording isolated product (153 mg).

### Example 80: Preparation of Cmpd59

Cmpd51 described in Example 24 was utilized as a reactant in a carbamoylation process:

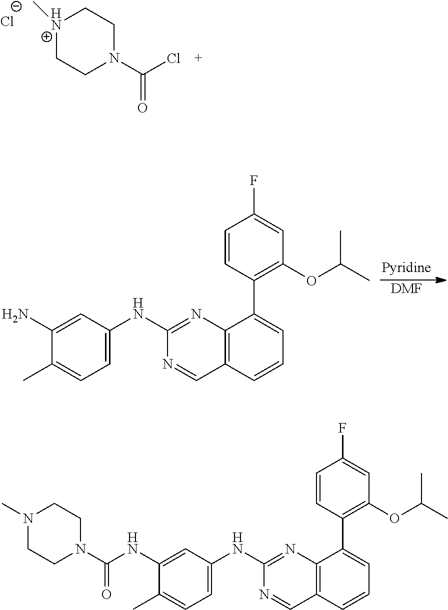

A 20 mL screw top scintillation vial containing N1-[8-(4-fluoro-2-isopropoxy-phenyl)quinazolin-2-yl]-4-methylbenzene-1,3-diamine (41 mg, 0.102 mmol) was charged with pyridine (1 mL). 4-methylpiperazin-4-ium-1-carbonyl chloride;chloride (41 mg, 0.204 mmol) was added and the suspension was stirred at room temperature for 20 hr. LCMS showed about 5% conversion. Another charge of 4-methylpiperazin-4-ium-1-carbonyl chloride;chloride (41 mg, 0.204 mmol) was added, followed by DMF (0.5 mL). The suspension was stirred at room temperature for another 24 hr (44 hr total). The temperature was raised to 40° C. and the mixture was stirred for 1 week. Conversion was about 65%. The reaction was diluted with excess iPrOAc and washed with water (3× washes). The homogenous organic phase was dried over MgSO₄, filtered and evaporated to a dark residue (50 mg). The residue was applied to a 1000 micrometer pTLC plate and eluted with 20:1 DCM/MeOH. The indicated band was collected (yellow solid, 11 mg).

### Example 81: Preparation of Cmpd76

An amidation process was performed utilizing Cmpd27 (prepared by a process described in Example 79) as a reactant:

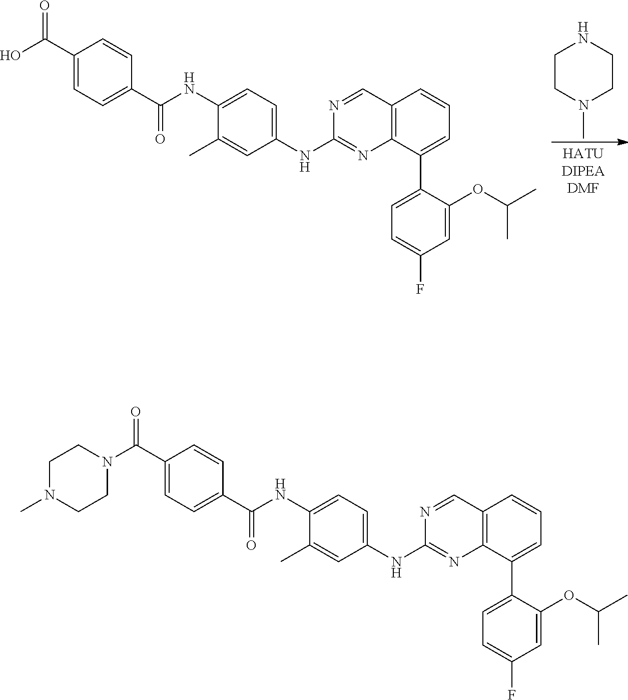

A 20 mL scintillation vial was charged with 4-[[4-[[8-(4-fluoro-2-isopropoxy-phenyl)quinazolin-2-yl]amino]-2-methylphenyl]carbamoyl]benzoic acid (21 mg, 0.038 mmol). DMF (1 mL) was added and the yellow mixture was stirred until dissolution was complete. DIEA (10 μL, 0.057 mmol) was added, followed by 1-methylpiperazine (7 μL, 0.057 mmol). HATU (22 mg, 0.057 mmol) was added and the solution stirred at room temperature for 4 hr. LCMS showed the reaction was complete. The reaction was diluted with excess iPrOAc and washed with water (4× washes). The organic phase was dried over MgSO₄, filtered and evaporated to a residue. The residue was suspended in EtOH (2 mL) and treated with 1N HCl (1 mL). The EtOH and most of the water were removed on the rotovap and under hi-vac. The remaining residue was lyophilized to an orange/brown residue which did not afford a solid on trituration with acetone. The residue was dissolved in MeOH and transferred to a freshly tared screw top scintillation vial, then evaporated and allowed to stand under high vacuum for 2 hr (20 mg).

### Example 82: Initial Results of Dose Escalation Study of Cmpd11 and Cmpd66

An initial non-GLP multi-dose toxicity study of Cmpd11 and Cmpd66 was performed, which included a comparison to COM1 and COM3 clinical compounds. Mice (6-week-old Balb/c females at study initiation (n=5) were dosed daily by IP injection, starting at the level sufficient to achieve in plasma the EC₅₀ (cytotoxic dose needed to kill 50% of cells) for CML line K562-Luc. Dose escalations commenced after each lower dose was determined to be tolerable in mice, up to the maximum tolerated dose (MTD) at which at least 50% of mice had to be euthanized from each group. Dose de-escalation was performed in some cases after an MTD was reached, so that the total number of distinct doses was 3-5 total. Daily dosing was planned for up to 28 days, which was followed by a 28-day recovery phase. Body weights were recorded 2× weekly for healthy mice followed by weekly in the recovery phase. Blood draws were taken, retro-orbitally, every other week (for a total of three blood draws per group) to determine the plasma levels of compounds throughout the duration of the study (unless mice were euthanized prior to completion of the 28-day dosing period). The first, second, and third bi-weekly blood draws were taken 1-hour post-dose (Cₘₐₓ), pre-dose (Cₘᵢₙ), and 4 hours post-dose, respectively. Any animals showing more than 9% weight loss were weighed every day and animals were euthanized after a weight loss of 15% or more or evidence of high distress (e.g., listless, hunched posture, high grimace score). Also, plasma samples were drawn at euthanasia. Control groups included vehicle only and no manipulation.

A summary of the study is illustrated in FIG. 14. There are six groups with 90 mice total and following is an overview of groups for the study.

| | Compound/ | |
|---|---|---|
| Group | Control | Administration |
| 1 | Vehicle control | Vehicle injected every day (dose N/A (n = 5)) |
| 2 | No vehicle | No manipulation (dose N/A (n = 5)) |
| | control | |
| 3 | Cmpd11 | Injected every day at 1.5 mg/kg (n = 5) (1X |
| | | EC₅₀); 4.5 mg/kg (n = 5) (3X |
| | | EC₅₀); and 18 mg/kg (n = 5) |
| | | (12X EC₅₀); studies of the following are |
| | | not complete or have not been |
| | | initiated: 22.5 mg/kg (n = 5) (15X |
| | | EC₅₀) and 27 mg/kg (n = 5) |
| | | (18X EC₅₀) |
| 4 | Cmpd66 | Injected every day at 1.5 mg/kg (n = 5) |
| | | (1X EC₅₀); 4.5 mg/kg (n = 5) |
| | | (3X EC₅₀); and 18 mg/kg |
| | | (n = 5) (12X EC₅₀); studies of |
| | | the following are not complete or |
| | | have not been initiated: 27 mg/kg (n = 5) |
| | | (18X EC₅₀) and 36 mg/kg (n = |
| | | 5) (24X EC₅₀) |
| 5 | COM1 | Injected every day at 9 mg/kg (n = 5) (1X |
| | | EC₅₀); 18 mg/kg (n = 5) |
| | | (2X EC₅₀); and 27 mg/kg |
| | | (n = 5) (3X EC₅₀); studies of the |
| | | following are not complete or |
| | | have not been initiated: 13.5 mg/kg (n = |
| | | 5) (1.5X EC₅₀) |
| 6 | COM3 | Injected every day at 1.5 mg/kg (n = 5) |
| | | (500X EC₅₀); 3.0 mg/kg |
| | | (n = 5) (1000X EC₅₀) and |
| | | 18 mg/kg (n = 5) (6000X EC₅₀) |

The following are criteria for dose escalation and staggering of group starting points. Animals in each group are monitored after the initial dose for 3-4 days, and if there is no observed dose-limiting toxicity (DLT), then a separate cohort of naïve animals are administered the next higher dose level. Note that each cohort of animals receives the same drug and drug dose for the duration of the 28-day dosing period unless euthanasia is required. Animals in the second, dose-escalated group, are then monitored for 3-4 days, and if there is no observed DLT, animals from a fresh cohort are administered the next higher dose level, while continuing with the former cohorts up to 5 dose levels total. Animals in each cohort then are monitored after the third dose for 7 days, and if there is no observed DLT, then animals are administered the next higher doses. If DLT is observed during a monitoring period for any dose level, a subsequent dose is reduced by a level that is intermediate between the highest tolerated level tested and the minimum toxic level tested.

The study is ongoing and not yet complete. Early results reflected acute toxicity (death of 50% of animals) of COM1 groups at doses of 18 mg/kg and 27 mg/kg. COM1 was well tolerated at a dose of 9 mg/kg through day 24 of the study. COM3 and Cmpd66 were well tolerated at the maximum dose of 18 mg/kg at day 14, and at 3 or 4.5 mg/kg, respectively, at day 21. Both COM3 and Cmpd66 were well tolerated at a dose of 1.5 mg/kg through day 24 of the study. Cmpd11 was well tolerated at dose levels of 1.5 and 4.5 mg/kg through day 24 and 14, respectively. Cmpd11, dosed at 18 mg/kg, resulted in expiration of 2 out of 5 animals (40%) by day 7. The early results indicate that both Cmpd11 and Cmpd66 are more tolerable than COM1 at the doses required to reach the in vitro K562-Luc EC₅₀ plasma levels in vivo.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described, the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A compound of Formula A1-3: 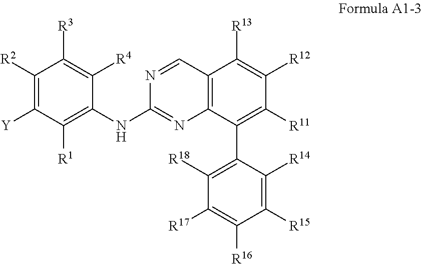
or a pharmaceutically acceptable salt, amide or ester thereof, wherein:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{b})C(O)—R^{Y} or —N(R^{b})C(O)—R^{V}—R^{Y};
R^{Y} is an optionally substituted heterocycloalkyl containing six ring atoms;
R^{V} is a substituted C1-C4 alkylene or unsubstituted C1-C4 alkylene;
R^{b}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

2. The compound of claim 1, wherein R¹, R², R³, R⁴, R¹¹, R¹², R¹³, R¹⁴, R¹⁵, R¹⁶, R¹⁷ and R¹⁸ each independently is hydrogen, optionally substituted C1-C4 alkyl, optionally substituted C1-C4 deuteroalkyl, optionally substituted C1-C4 haloalkyl; optionally substituted C1-C4 alkylamino, halo or optionally substituted C1-C4 alkoxy.

3. The compound of claim 1 or 2, wherein R¹, R², R³, R⁴, R¹¹, R¹², R¹³, R¹⁴, R¹⁵, R¹⁶, R¹⁷ and R¹⁸ each independently is hydrogen, unsubstituted C1-C4 alkyl, Cl, F, CF₃, CD₃, unsubstituted C1-C4 alkoxy, methoxy, isopropyloxy or dimethylamino.

4. The compound of any one of claims 1-3, wherein (i) R¹⁴ and R¹⁶ each independently is hydrogen, unsubstituted C1-C4 alkyl, Cl, F, CF₃, CD₃, unsubstituted C1-C4 alkoxy, methoxy, isopropyloxy or dimethylamino; (ii) R¹⁴ is unsubstituted C1-C4 alkoxy; (iii) R¹⁴ is methoxy or isopropyloxy; (iv) R¹⁶ is chloro or fluoro; and/or (v) R¹⁶ is fluoro.

5. The compound of any one of claims 1-4, wherein:
one, two, three or four of R¹, R¹¹, R¹² and R¹³ each is hydrogen;
one, two or three of R¹⁵, R¹¹ and R¹⁸ each is hydrogen;
one or two of R³ and R⁴ each is hydrogen; and/or
R^{b} is hydrogen.

6. The compound of any one of claims 1-5, wherein R² is methyl, ethyl, methoxy or ethoxy, and R¹, R³ and R⁴ each is hydrogen.

7. The compound of any one of claims 1-6, wherein R^{V} is unsubstituted ethylene or unsubstituted methylene.

8. The compound of any one of claims 1-7, wherein (i) R^{p}, R^{q}, R^{r} and R^{s} each independently is hydrogen or methyl; and/or (ii) R^{u} is ethyl or methyl.

9. The compound of any one of claims 1-8, wherein R^{Y} is an optionally substituted piperidinyl, optionally substituted piperazinyl or optionally substituted morpholinyl.

10. The compound of any one of claims 1-9, wherein R^{Y} is a substituted piperidinyl or substituted piperazinyl, which optionally is substituted by an optionally substituted C1-C6 alkyl, ethyl or methyl at one, two or three ring atoms.

11. The compound of any one of claims 1-9, wherein R^{Y} is an unsubstituted morpholinyl.

12. The compound of any one of claims 1-9, wherein R^{Y} is of Formula D1: 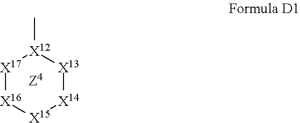
wherein:
X¹² is N; X¹³ is C(R⁵)R^{5A}; X¹⁴ is C(R⁶)R^{6A}; X¹⁵ is N—R^{7B}, X¹⁶ is C(R⁸)R^{8A}, and X¹⁷ is C(R⁹)R^{9A}; or
X¹² is C—R^{aA}; X¹³ is C(R⁵)R^{5A}; X¹⁴ is C(R⁶)R^{6A}; X¹⁵ is N—R^{7B}; X¹⁶ is C(R⁸)R^{8A}; and X¹⁷ is C(R⁹)R^{9A};
R^{aA}, R⁵, R^{5A}, R⁶, R^{6A}, R⁸, R^{8A}, R⁹ and R^{9A} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{k}R^{j}, —C(O)R^{z}, —C(O)OH, —C(O)OR^{u}, —B(OH)₂, hydroxy, halo, nitro or cyano;
R^{7B} is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, optionally substituted C5-C6 cycloalkyl, optionally substituted heterocycloalkyl containing 5 or 6 ring member atoms, or —C(O)OR^{u};
R^{g}, R^{h}, R^{k} and R^{j} each independently is hydrogen or optionally substituted C1-C6 alkyl; and
R^{u} is an optionally substituted C1-C6 alkyl.

13. The compound of claim 12, wherein Y is —N(R^{b})C(O)—R^{Y}; X¹² is C—R^{aA}; X¹³ is C(R⁵)R^{5A}; X¹⁴ is C(R⁶)R^{6A}; X¹⁵ is N—R^{7B}; X¹⁶ is C(R⁸)R^{8A}; and X¹⁷ is C(R⁹)R^{9A}.

14. The compound of claim 12, wherein Y is —N(R^{b})C(O)—R^{V}—R^{Y}; X¹² is N; X¹³ is C(R⁵)R^{5A}; X¹⁴ is C(R⁶)R^{6A}; X¹⁵ is N—R^{7B}, X¹⁶ is C(R⁸)R^{8A}, and X¹⁷ is C(R⁹)R^{9A}.

15. The compound of any one of claims 12-14, wherein (i) R^{aA}, R⁵, R^{5A}, R⁶, R^{6A}, R^{7B}R⁸, R^{BA}, R⁹ and R^{9A} each independently is hydrogen or optionally substituted C1-C4 alkyl; (ii) R^{u} is an optionally substituted C1-C4 alkyl; (iii) R^{g}, R^{h}, R^{k} and R^{j} each independently is hydrogen or optionally substituted C1-C4 alkyl; (iv) the optionally substituted C1-C4 alkyl of (i), (ii) or (iii) is an unsubstituted C1-C4 alkyl; and/or (v) the unsubstituted C1-C4 alkyl of (iv) is butyl, tert-butyl, iso-butyl, propyl, isopropyl, ethyl or methyl.

16. The compound of any one of claims 12-15, wherein R^{aA}, R⁵, R^{5A}, R⁶, R^{6A}, R^{7B}, R⁸, R^{8A}, R⁹ and R^{9A} each independently is hydrogen or unsubstituted C1-C4 alkyl.

17. The compound of any one of claims 12-16, wherein R^{aA}, R⁵, R^{5A}, R⁶, R^{6A}, R, R^{8A}R⁹ and R^{9A} each is hydrogen and R^{7B} is methyl.

18. The compound of any one of claims 1-10 and 12-17, wherein Y is:

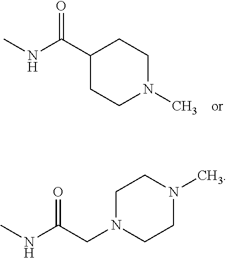

19. A compound or pharmaceutically acceptable salt thereof, wherein the compound is chosen from:
N-(5-((8-(4-fluoro-2-isopropoxyphenyl)quinazolin-2-yl)amino)-2-methylphenyl)-1-methylpiperidine-4-carboxamide; or
(S)—N-(5-((8-(4-fluoro-2-isopropoxyphenyl)quinazolin-2-yl)amino)-2-methylphenyl)-1-methylpyrrolidine-2-carboxamide; or
N-(5-((8-(4-fluoro-2-isopropoxyphenyl)quinazolin-2-yl)amino)-2-methylphenyl)-1-methylazetidine-3-carboxamide; or
N-(5-((8-(4-fluoro-2-isopropoxyphenyl)quinazolin-2-yl)amino)-2-methylphenyl)tetrahydro-2H-pyran-4-carboxamide; or
(R)—N-(5-((8-(4-fluoro-2-isopropoxyphenyl)quinazolin-2-yl)amino)-2-methylphenyl)-1-methylpyrrolidine-2-carboxamide; or
N-(5-((8-(4-fluoro-2-isopropoxyphenyl)quinazolin-2-yl)amino)-2-methylphenyl)-2-(4-methylpiperazin-1-yl)acetamide; or
N-(5-((8-(4-fluoro-2-isopropoxyphenyl)quinazolin-2-yl)amino)-2-methylphenyl)-4-methylpiperazine-1-carboxamide.

20. A compound or pharmaceutically acceptable salt thereof, wherein the compound is chosen from:
N-(5-((8-(4-fluoro-2-isopropoxyphenyl)quinazolin-2-yl)amino)-2-methylphenyl)-1-methylpiperidine-4-carboxamide; or
N-(5-((8-(4-fluoro-2-isopropoxyphenyl)quinazolin-2-yl)amino)-2-methylphenyl)-2-(4-methylpiperazin-1-yl)acetamide.

21. A compound of Formula A1 or Formula A2: 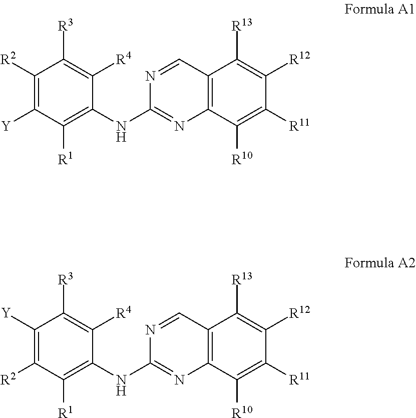
or a pharmaceutically acceptable salt, amide or ester thereof, wherein:
R¹, R², R³ and R⁴ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino or amido;
Y is —N(R^{b})C(O)—R^{Y}, —C(O)N(R^{b})—R^{YA}, —N(R^{b})—CH₂—R^{Y}; —CH₂—N(R^{b})R^{Y}, —N(R^{a})C(O)—R^{V}—N(R^{b})R^{Y}, —N(R^{b})C(O)—R^{V}—R^{Y}, —N(R^{a})R^{b} or of Formula F;
R^{a} is hydrogen, optionally substituted alkyl, optionally substituted alkynyl or of Formula F: 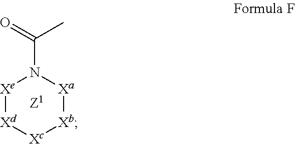
R^{b} is hydrogen or optionally substituted alkyl;
R^{V} is an optionally substituted alkylene;
R^{Y} is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted hydroxyalkyl, optionally substituted mercaptoalkyl, optionally substituted aminoalkyl, optionally substituted cycloalkyl, optionally substituted heterocycloalkyl, optionally substituted cycloalkylalkyl, optionally substituted heterocycloalkylalkyl, optionally substituted aryl, optionally substituted arylaminoalkyl, optionally substituted aryloxyalkyl, optionally substituted arylthioalkyl or optionally substituted arylalkyl, optionally substituted heteroaryl, optionally substituted heteroarylaminoalkyl, optionally substituted heteroaryloxyalkyl, optionally substituted heteroarylthioalkyl, optionally substituted heteroarylalkyl or is of Formula F;
R^{YA} is an optionally substituted aryl, optionally substituted arylaminoalkyl, optionally substituted aryloxyalkyl, optionally substituted arylthioalkyl or optionally substituted arylalkyl, optionally substituted heteroaryl, optionally substituted heteroarylaminoalkyl, optionally substituted heteroaryloxyalkyl, optionally substituted heteroarylthioalkyl or optionally substituted heteroarylalkyl;
Z¹ is an optionally substituted heterocycloalkyl, X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A}; and R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R¹⁰, R¹¹, R¹² and R¹³ each independently is hydrogen, optionally substituted alkyl, optionally substituted alkenyl, optionally substituted alkynyl, optionally substituted alkoxy, optionally substituted heteroalkyl, optionally substituted haloalkyl, optionally substituted deuteroalkyl, optionally substituted alkylthio, optionally substituted alkylamino, optionally substituted hydroxyalkyl, optionally substituted mercaptoalkyl, optionally substituted aminoalkyl, —C(O)R^{z}, —C(O)OH, —C(O)OR^{u}, —B(OH)₂, hydroxy, halo, cyano, nitro, amino, amido, R^{W} or —W—R^{W};
R^{W} is an optionally substituted cycloalkyl, optionally substituted cycloalkylalkyl, optionally substituted heterocycloalkyl, optionally substituted heterocycloalkylalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl;
W is an optionally substituted alkylene, optionally substituted alkynyl, amino, amido, —O—, —S—, —S(O)— or —SO₂—;
R^{z} is hydrogen or R^{u}; and
R^{u} is an optionally substituted alkyl, optionally substituted hydroxyalkyl, optionally substituted mercaptoalkyl, optionally substituted aminoalkyl, optionally substituted cycloalkyl, optionally substituted heterocycloalkyl, optionally substituted aryl, optionally substituted heteroaryl, optionally substituted arylaminoalkyl, optionally substituted aryloxyalkyl, optionally substituted arylthioalkyl, optionally substituted heteroarylaminoalkyl, optionally substituted heteroaryloxyalkyl, optionally substituted heteroarylthioalkyl, optionally substituted arylalkyl or optionally substituted heteroarylalkyl;
with the proviso that the compound is not of Formula X1, not of Formula X2, not of Formula X3 and not of Formula X4: 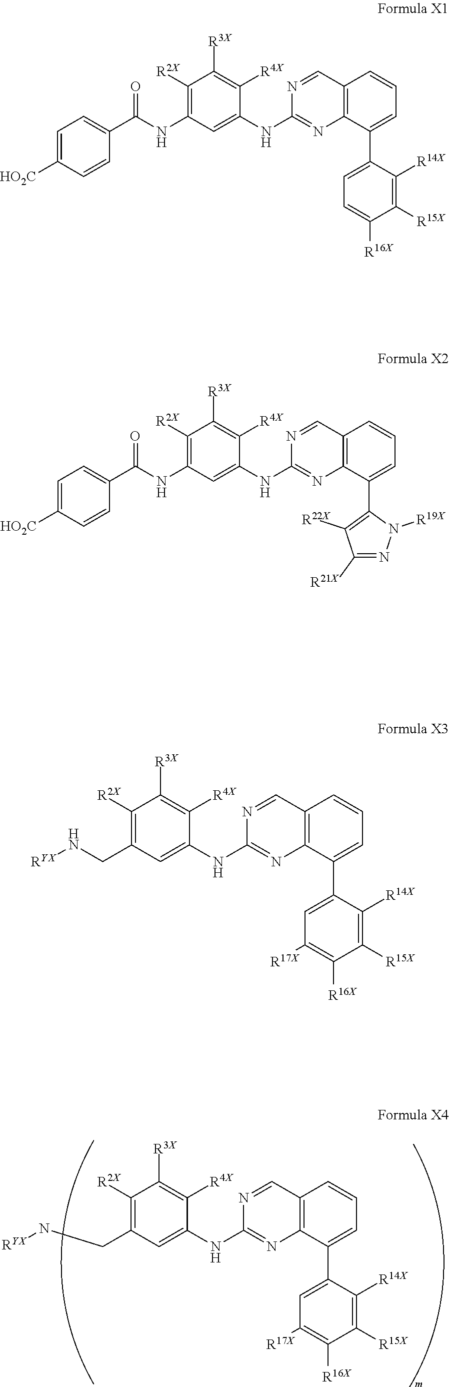
wherein: R^{2X}, R^{3X} and R^{4X} each independently is hydrogen, optionally substituted C1-C4 alkyl or optionally substituted C1-C4 alkoxy; R^{14X}, R^{15X}, R^{16X} and R^{17X} each independently is hydrogen, optionally substituted C1-C4 alkyl, optionally substituted C1-C4 alkoxy, or halo; R^{19X}, R^{21X} and R^{22X} each independently is hydrogen or optionally substituted C1-C6 alkyl; m is an integer of 1 or 2; R^{YX} is methyl or 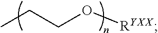
n is an integer of 1 to 10; and R^{YXX} is hydrogen, optionally substituted alkyl or optionally substituted amidoalkyl.

22. The compound of claim 21, which is of Formula A1-3: 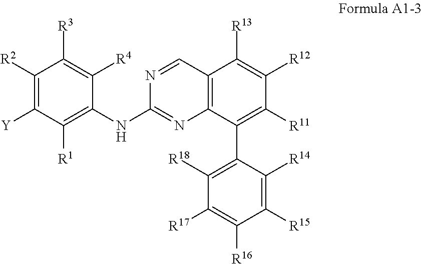
or a pharmaceutically acceptable salt, amide or ester thereof, wherein:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{a})R^{b};
R^{a}, R^{b}, R^{p}, R^{q}, R^{r} and R^{s} each independently is hydrogen or optionally substituted C1-C6 alkyl;
R^{z} is hydrogen or R^{u}; and
R^{u} is or optionally substituted C1-C6 alkyl.

23. The compound of claim 21, which is of Formula A1-3: 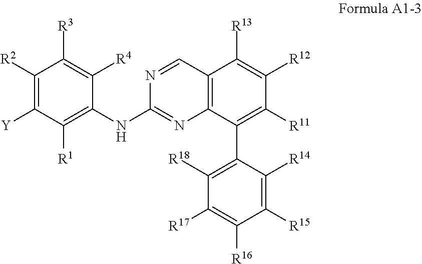
or a pharmaceutically acceptable salt, amide or ester thereof, wherein:
R¹, R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
Y is —N(R^{b})C(O)—R^{Y};
R^{Y} is an optionally substituted alkenyl;
R² is hydrogen, optionally substituted C1-C6 alkyl, unsubstituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —N(H)R^{r}, —NH₂, —C(O)R^{z}, —C(O)OH, —C(O)OR^{u}, —B(OH)₂, hydroxy, halo, nitro or cyano;
R^{b}, R^{g}, R^{h}, R^{j}, R^{k}, R^{p} and R^{q} each independently is hydrogen or optionally substituted C1-C6 alkyl;
R^{r} and R^{s} each independently is hydrogen or unsubstituted C1-C6 alkyl;
R^{z} is hydrogen or R^{u}; and
R^{u} is an optionally substituted C1-C6 alkyl.

24. The compound of claim 21, which is of Formula A1-8: 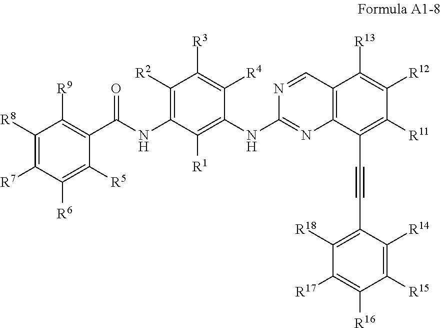
or a pharmaceutically acceptable salt, amide or ester thereof, wherein:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
R⁵, R⁶, R⁷, R⁸ and R⁹ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{g}, R^{h}, R^{j}, R^{k}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

25. The compound of claim 21, which is of Formula A1-5: 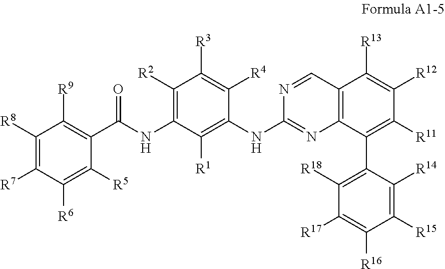
or a pharmaceutically acceptable salt, amide or ester thereof, wherein:
R⁷ is —C(O)N(R^{c}R^{d}) or is of Formula F: 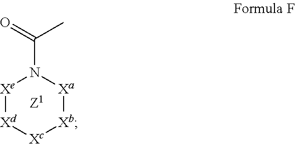
R¹, R², R³, R⁴, R⁵, R⁶, R⁸, R⁹, R¹¹, R¹², R¹³, R¹⁴, R¹⁵, R¹⁶, R¹¹ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Z¹ is an optionally substituted heterocycloalkyl; X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{C}, R^{d}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

26. The compound of claim 21, which is of Formula A1-3: 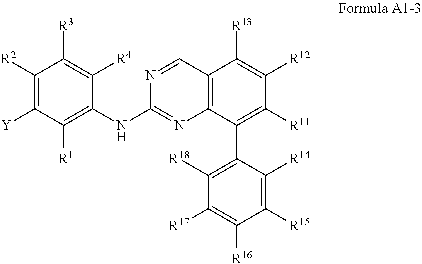
or a pharmaceutically acceptable salt, amide or ester thereof, wherein:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is —N(R^{b})C(O)—R^{Y};
R^{Y} is an unsubstituted C1-C6 alkyl or unsubstituted C1-C6 deuteroalkyl;
R^{b}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

27. The compound of claim 21, which is of Formula A1-6: 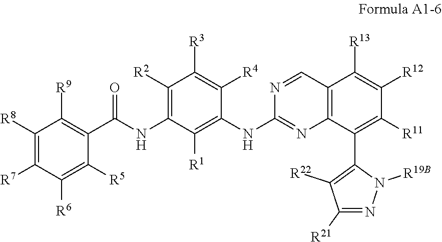
or a pharmaceutically acceptable salt, amide or ester thereof, wherein:
R¹, R², R³, R⁴, R¹¹, R¹², R¹³, R²¹ and R²² each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{g}C(O)N(R^{h})—, —C(O)N(R^{g}R^{h}), —NR^{j}R^{k}, —C(O)R^{z}, —C(O)OH, —C(O)OR^{u}, —B(OH)₂, hydroxy, halo, nitro or cyano;
R⁵, R⁶, R⁸ and R⁹ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R⁷ is R^{c}C(O)N(R^{d})— or —C(O)N(R^{c}R^{d});
each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{p}), —NR^{r}R^{s}, —C(O)R^{z}, —C(O)OH, —C(O)OR^{u}, —B(OH)₂, hydroxy, halo, nitro or cyano;
R^{19B} is hydrogen, optionally substituted C1-C6 alkyl, optionally substituted C1-C4 alkyl, butyl, tert-butyl, iso-butyl, propyl, iso-propyl, ethyl or methyl;
R^{c}, R^{d}, R^{g}, R^{h}, R^{j}, R^{k}, R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

28. The compound of claim 21, which is of Formula A1-3: 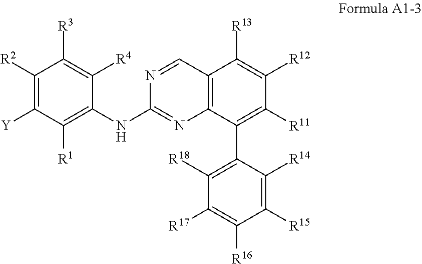
or a pharmaceutically acceptable salt, amide or ester thereof, wherein:
R¹, R², R³, R⁴, R¹¹, R¹², R¹³, R¹⁴, R¹⁵, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{p})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
Y is of Formula F: 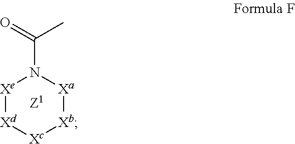
Z¹ is an optionally substituted heterocycloalkyl; X^{a} is C(R⁴³)R^{43A}; X^{b} is C(R⁴⁴)R^{44A}; X^{c} is C(R⁴⁵)R^{45A}, N—R^{45B}, O, S, S(O) or SO₂; X^{d} is C(R⁴⁶)R^{46A}; and X^{e} is C(R⁴⁷)R^{47A};
R⁴³, R⁴⁴, R⁴⁵, R⁴⁶, R⁴⁷, R^{43A}, R^{44A}, R^{45A}, R^{46A}, R^{47A} and R^{45B} each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl;
R^{p}, R^{q}, R^{r} and R^{s} each independently is hydrogen or optionally substituted C1-C6 alkyl;
R^{z} is hydrogen or R^{u}; and
R^{u} is or optionally substituted C1-C6 alkyl.

29. The compound of claim 21, which is of Formula A1-2: 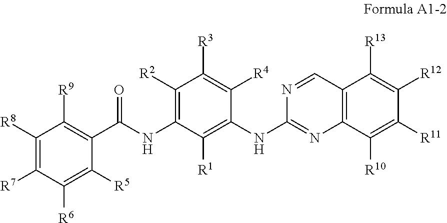
or a pharmaceutically acceptable salt, amide or ester thereof, wherein:
R¹⁰ is of Formula B2: 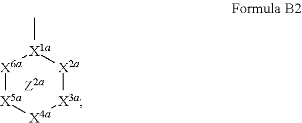
Z^{2a} is heteroaryl; and (i) X^{1a} is C, X^{2a} is C—R¹⁴, X^{3a} is N, X^{4a} is C—R¹⁶, X^{5a} is N, and X^{6a} is C—R¹⁸; or (ii) X^{1a} is C, X^{2a} is C—R¹⁴, X^{3a} is N, X^{4a} is C—R¹⁶, X^{5a} is C—R¹⁷, and X^{6a} is C—R¹⁸, and the X^{3a} nitrogen and R¹⁶ together are joined as a fused optionally substituted heteroaryl containing five ring member atoms;
R¹, R², R³, R⁴, R⁵, R⁶, R⁷, R⁸, R⁹, R¹¹, R¹², R¹³, R¹⁴, R¹⁶, R¹⁷ and R¹⁸ each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{P}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

30. The compound of claim 21, which is of Formula A1-2: 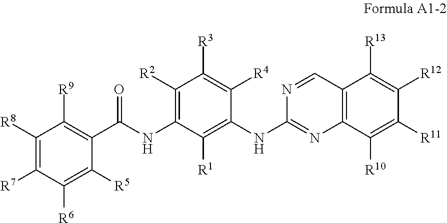
or a pharmaceutically acceptable salt, amide or ester thereof, wherein:
R¹¹ is of Formula C2: 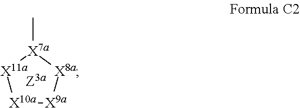
Z^{3a} is heteroaryl; and X^{7a} is C; X^{8a} is N or N—R^{19B}; X^{9a} is N or N—R^{20B}; X^{10a} is C—R²¹; and
X^{11a} is C—R²²;
R¹, R², R³, R⁴, R⁵, R¹, R⁷, R¹, R⁹, R¹⁰, R¹², R¹³, R²¹ and R²² each independently is hydrogen, optionally substituted C1-C6 alkyl, optionally substituted C1-C6 alkoxy, optionally substituted C1-C6 heteroalkyl, optionally substituted C1-C6 haloalkyl, optionally substituted C1-C6 deuteroalkyl, optionally substituted C1-C6 alkylthio, optionally substituted C1-C6 alkylamino, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH, —C(O)OR^{u}, —B(OH)₂, hydroxy, halo, nitro or cyano;
R^{19B} and R^{20B} each independently is hydrogen, optionally substituted C1-C6 alkyl, optionally substituted C1-C6 heteroalkyl, optionally substituted C1-C6 haloalkyl, optionally substituted C1-C6 deuteroalkyl, optionally substituted C1-C6 hydroxyalkyl, optionally substituted C1-C6 mercaptoalkyl, optionally substituted C1-C6 aminoalkyl, R^{p}C(O)N(R^{q})—, —C(O)N(R^{p}R^{q}), —NR^{r}R^{s}, —C(O)R^{z}, —C(O)OH or —C(O)OR^{u};
R^{p}, R^{q}, R^{r} and R^{s} each independently is hydrogen or optionally substituted C1-C6 alkyl;
R^{z} is hydrogen or R^{u}; and
R^{u} is an optionally substituted C1-C6 alkyl.

31. A pharmaceutical composition comprising a compound of any one of claims 1-30 and a pharmaceutically acceptable excipient.

32. Use of a compound or composition or any one of claims 1-31, for inhibiting a protein kinase.

33. Use of a compound or composition of any one of claims 1-31, for treatment of a medical condition or for preparation of a medicament for treatment of a medical condition.

34. The use of claim 33, wherein the medical condition is a cancer.

35. The use of claim 34, wherein the cancer is a leukemia.

36. The use of claim 35, wherein the leukemia is acute myeloid leukemia (AML), chronic myeloid leukemia (CML) or acute lymphoblastic leukemia (ALL).

37. The use of claim 36, wherein the CML is chronic phase CML (CML-CP), acute phase CML (CML-AP) and blast phase CML (CML-BP).

38. The use of claim 36, wherein the ALL is a relapsed and/or refractory ALL (R/R ALL).

39. The use of claim 36 or 38, wherein the ALL is Philadelphia chromosome-positive ALL, Philadelphia chromosome-positive-like-ALL, B-cell acute lymphoblastic leukemia (B-ALL) or T-cell acute lymphoblastic leukemia (T-ALL).

40. The use of claim 39, wherein the cancer is B-ALL, and optionally the B-ALL is a TCF3-HLF-positive B-ALL, or optionally is a TCF3-HLF-positive acute B-ALL

41. The use of claim 39, wherein the cancer is T-ALL, and optionally the T-ALL is a R/R T-ALL.

42. The use of any one of claims 33-41, wherein the compound is of any one of claims 1-20.
